(19) United States
(12) Patent Application Publication
(10) Pub. No.: US 2025/0043001 A1
(43) Pub. Date: Feb. 6, 2025

(54) ANTI-CD28 COMPOSITIONS

(71) Applicant: Xencor, Inc., Pasadena, CA (US)

(72) Inventors: Gregory Moore, Azusa, CA (US); Viralkumar Rameshkumar Davra, Chino, CA (US); Dong Hyun Nam, Monterey Park, CA (US); Veronica Gusti Zeng, Duarte, CA (US); Michael Hedvat, Encino, CA (US); Jing Qi, Arcadia, CA (US); Rumana Rashid, Temple City, CA (US); Patrick Arpp, Pasadena, CA (US); Ernesto Delgado, Rancho Cucamonga, CA (US); ChaoFang Tan, Arcadia, CA (US); Heather Preciado Jimenez, Upland, CA (US); Charles G. Bakhit, La Verne, CA (US); Juan Diaz, Anaheim Hills, CA (US)

(21) Appl. No.: 18/605,762

(22) Filed: Mar. 14, 2024

Related U.S. Application Data
(60) Provisional application No. 63/490,167, filed on Mar. 14, 2023.
(60) Provisional application No. 63/496,374, filed on Apr. 14, 2023.
(60) Provisional application No. 63/490,171, filed on Mar. 14, 2023.
(60) Provisional application No. 63/495,989, filed on Apr. 13, 2023.
(60) Provisional application No. 63/578,592, filed on Aug. 24, 2023.

(51) Int. Cl. C07K 16/28; C07K 16/30; C07K 16/46

(52) U.S. Cl. CPC C07K 16/2818; C07K 16/3069; C07K 16/468; C07K 2317/31; C07K 2317/51; C07K 2317/515; C07K 2317/52; C07K 2317/622

(57) ABSTRACT

Provided herein are novel anti-CD28 x anti-STEAP1 and anti-CD28 x anti-CEACAM5 antibodies and methods of using such antibodies for the treatment of related cancers.

### PRIORITY CLAIM

This application claims priority to and the benefit of United States Provisional Application Nos. 63/490,167, filed Mar. 14, 2023; 63/496,374, filed Apr. 14, 2023; 63/490,171, filed Mar. 14, 2023; 63/495,989, filed Apr. 13, 2023; and 63/578,592, filed Aug. 24, 2023, the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on May 7, 2024, is named 067461-5313-US_SL.xml and is 3,451,619 bytes in size.

### BACKGROUND

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. One particular approach for bispecific antibodies is to engineer a first binding domain which engages CD3 and a second binding domain which engages an antigen associated with or upregulated on cancer cells so that the bispecific antibody redirects CD3⁺ T cells to destroy the cancer cells.

TILs, however, lose their cytotoxic ability over time due to upregulation of inhibitory immune checkpoints. While checkpoint blockade has demonstrated increased clinical response rates relative to other treatment options, many patients still fail to achieve a response to checkpoint blockade. Engagement of costimulatory receptors on TILs could provide a positive signal capable of overcoming negative signals of immune checkpoints. Preclinical and clinical studies of agonistic costimulatory receptor antibodies have indeed demonstrated that agonism of costimulatory receptors can result in impressive anti-tumor responses, activating T cells to attack tumor cells.

It is also important for cancer therapy to enhance anti-tumor activity by specifically destroying tumor cells while minimizing peripheral toxicity. In this context, it is crucial that only T cells in the presence of the target tumor cells are provided a costimulatory signal. However, agonism of costimulatory receptors with monospecific full-length antibodies is likely nondiscriminatory with regards to TILs vs. peripheral T cells vs. autoantigen-reactive T cells that contribute to autoimmune toxicities.

Six-Transmembrane Epithelial Antigen of the Prostate 1 (STEAP1 ) is a cell surface antigen that is highly expressed in several carcinomas (e.g., prostate, breast, and bladder cancer). Carcinoembryonic antigen-related cell adhesion molecule 5 (CEACAM5) is a cell surface glycoprotein, and member of the carcinoembryonic (CEA) antigen family. CEACAM5 is overexpressed in numerous tumor types including gastrointestinal, breast, and lung. Since these antigens are overexpressed on a variety of tumors, STEAP1 and CEACAM5 are potential candidates for targeted cancer therapeutic development.

While immunotherapies targeting STEAP1 and CEACAM5 have been tried, there still remains a need for novel immune response enhancing compositions for the treatment of STEAP1 -associated and CEACAM5-associated cancers.

### SUMMARY

Provided herein are novel anti-CD28 x anti-STEAP1 and anti-CD28 x anti-CEACAM5 antibodies and methods of using such antibodies for the treatment of related cancers (e.g., STEAP1 or CEACAM5-associated cancers). Subject anti-CD28 x anti-STEAP1 antibodies are capable of agonistically binding to CD28 costimulatory molecules on T cells and STEAP1 on tumor cells. Subject anti-CD28 x anti-CEACAM5 antibodies are capable of agonistically binding to CD28 costimulatory molecules on T cells and CEACAM5 on tumor cells. Thus, such antibodies selectively enhance anti-tumor activity at tumor sites while minimizing peripheral toxicity. The subject antibodies provided herein are particularly useful in combination with other anti-cancer therapies, including, for example, bispecific antibodies for the treatment of STEAP1 - and CEACAM5-associated cancers.

In a first aspect, provided herein is an anti-CD28 x anti-STEAP1 heterodimeric antibody that comprises a) a first monomer; b) a second monomer; and c) a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VHT is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VHT and the VLT together form a first antigen binding domain (ABD) and the VH2 and the VL2 together form a second ABD. Further, one of the first ABD and the second ABD is a CD28 binding domain and the other of the first ABD and second ABD is Six Transmembrane Epithelial Antigen of Prostate 1 (STEAP1 ) binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABD is the STEAP1 binding domain and the second ABD is the CD28 binding domain. In some embodiments, VH1 and VL1 are selected from one of the following: a) a VH and VL of any of the STEAP1 binding domains from FIG. 22 or 39 or a variant thereof; or b) a VH from FIG. 31 and a VL from FIG. 32 or a variant thereof. In some embodiments, VH2 and VL2 are selected from one of the following: 1) a VH and VL of any of the CD28 binding domains from FIGS. 15, 18, and 21 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D and the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is an anti-CD28 x anti-STEAP1 heterodimeric antibody that comprises a) a first monomer; b) a second monomer; c) a first light chain; and d) a second light chain. The first monomer comprises, from N-terminal to C-terminal, VH1-CH1-first domain linker-scFv-second domain linker-CH2-CH3, wherein VH1 is a first variable heavy domain, and CH2-CH3 is a first Fc domain. The second monomer comprises, from N-terminal to C-terminal, a VH1-CHT-hinge-CH2-CH3, wherein CH2-CH3 is a second Fc domain. The first light chain comprises, from N-terminal to C-terminal, VL1-CL, wherein VL1 is a first variable light domain and CL is a constant light domain. The second light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain and the VH1 of the second monomer and the VL1 of the second light chain each form a first antigen binding domain (ABD), and the VH2 and the VL2 form a second ABD. Further, the first ABDs are STEAP1 binding domains and the second ABD is a CD28 binding domain, or the first ABDs are CD28 binding domains and the second ABD is a STEAP1 binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the STEAP1 binding domains and the second ABD is the CD28 binding domain. In some embodiments, VH1 and VL1 are selected from one of the following: a) a VH and VL of any of the STEAP1 binding domains from FIG. 22 or 39 or a variant thereof; or b) a VH from FIG. 31 and a VL from FIG. 32, or a variant thereof. In some embodiments, VH2 and VL2 are selected from one of the following: 1) a VH and VL of any of the CD28 binding domains from FIGS. 15, 18, and 21 or a variant thereof; or 2) (i) a VH from FIGS. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fe domains comprise heterodimerization skew variants S364K/E357Q: L368D/K370S, wherein numbering is according to EU numbering

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D and the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is an anti-CD28 x anti-STEAP1 heterodimeric antibody that comprises a) a first monomer; b) a second monomer c) a first light chain; and d) a second light chain. The first monomer comprises, from N-terminal to C-terminal, VH1-CH1-hinge-CH2-CH3-domain linker-scFv, wherein VH1 is a first variable heavy domain, and CH2-CH3 is a first Fc domain. The second monomer comprises, from N-terminal to C-terminal, a VH1-CHT-hinge-CH2-CH3, wherein VH1 is a first variable heavy domain and CH2-CH3 is a second Fc domain. The first light chain comprises, from N-terminal to C-terminal, VL1-CL, wherein VL1 is a first variable light domain and CL is a constant light domain. The second light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain and the VH1 of the second monomer and the VL1 of the second light chain each form a first antigen binding domain (ABD), and the VH2 and the VL2 form a second ABD. Further, the first ABDs are STEAP1 binding domains and the second ABD is a CD28 binding domain, or the first ABDs are CD28 binding domains and the second ABD is a STEAP1 binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the STEAP1 binding domains and the second ABD is the CD28 binding domain. In some embodiments, VH1 and VL1 are selected from one of the following: a) a VH and VL of any of the STEAP1 binding domains from FIG. 22 or 39 or a variant thereof; or b) a VH from FIG. 31 and a VL from FIG. 32, or a variant thereof. In some embodiments, VH2 and VL2 are selected from one of the following: 1) a VH and VL of any of the CD28 binding domains from FIGS. 15, 18, and 21 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D and the first Fc domain comprises amino acid variants E233P/L234V/L235A/G236del/S267K/S364K/E357Q, wherein numbering is according to EU numbering. In exemplary embodiments, the first and second variant Fe domains each further comprise amino acid variants 428L/434S.

In some embodiments, the scFv linker is GKPGSGKPGSGKPGSGKPGS (SEQ ID NO: 24).

In another aspect, provided herein is an anti-CD28 x anti-STEAP1 heterodimeric antibody that comprises a) a first monomer; b) a second monomer; c) a first light chain; and d) a second light chain. The first monomer comprises, from N-terminal to C-terminal, a VH1-CH1-domain linker-VH1-CHT-hinge-CH2-CH3, wherein each of the VHTs is a first variable heavy domain, and CH2-CH3 is a first Fc domain. The second monomer comprises, from N-terminal to C-terminal, an scFv-domain linker-CH2-CH3, wherein VH1 is a first variable heavy domain and CH2-CH3 is a second Fc domain. The first light chain comprises, from N-terminal to C-terminal, VL1-CL, wherein VL1 is a first variable light domain and CL is a constant light domain. The second light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain and the VH1 of the second monomer and the VL1 of the second light chain each form a first antigen binding domain (ABD), and the VH2 and the VL2 form a second ABD. Further, the first ABDs are STEAP1 binding domains and the second ABD is a CD28 binding domain, or the first ABDs are CD28 binding domains and the second ABD is a STEAP1 binding domain. In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the STEAP1 binding domains and the second ABD is the CD28 binding domain. In some embodiments, VH1 and VL1 are selected from one of the following: a) a VH and VL of any of the STEAP1 binding domains from FIG. 22 or 39 or a variant thereof; or b) a VH from FIG. 31 and a VL from FIG. 32, or a variant thereof. In some embodiments, VH2 and VL2 are selected from one of the following: 1) a VH and VL of any of the CD28 binding domains from FIGS. 15, 18, and 21 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fe domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is a bispecific antibody that comprises: a) a STEAP1 binding domain comprising i) a first variable heavy domain (VH1), and ii) a first variable light domain (VL1); and b) an anti-CD28 binding domain comprising i) a second variable heavy domain (VH2), and ii) a second variable light domain (VL2). In some embodiments, VH1 and VL1 are selected from one of the following: a) a VH and VL of any of the STEAP1 binding domains from FIG. 22 or 39 or a variant thereof; or b) a VH from FIG. 31 and a VL from FIG. 32, or a variant thereof. In some embodiments, VH2 and VL2 are selected from one of the following: 1) a VH and VL of any of the CD28 binding domains from FIGS. 15, 18, and 21 or a variant thereof; or 2) (i) a VH from FIG. 15 or 16 or a variant thereof; and (ii) a VL from FIGS. 15, 17 or 82 or a variant thereof.

In some embodiments of the bispecific antibodies, the first Fc domain and second Fc domain are each variant Fc domains.

In some embodiments of the bispecific antibodies, the first and second Fc domains comprise a set of heterodimerization skew variants selected from the following heterodimerization variants: S364K/E357Q: L368D/K370S; S364K: L368D/K370S; S364K : L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the pI variants N208D/Q295E/N384D/Q418E/N421D, wherein numbering is according to EU numbering.

Also provided herein are nucleic acid compositions comprising nucleic acids encoding the antibodies described herein, expression vector compositions that include such nucleic acids, host cells for making the antibodies that comprise the expression vector compositions, and methods of making the antibodies.

In another aspect, provided herein is a method of treating a STEAP1 -associated cancer in a patient in need thereof, comprising administering to the patient an anti-CD28 x anti-STEAP1 bispecific antibody as described herein.

In another aspect, provided herein is a method of treating a STEAP1 -associated cancer in a patient in need thereof, comprising administering to the patient an anti-CD28 x anti-STEAP1 bispecific antibody as described herein; and an anti-CD3 x anti-STEAP1 bispecific antibody.

In another aspect, provided herein are compositions comprising a STEAP1 antigen binding domain (ABD). In some embodiments, the STEAP1 includes a) a variable heavy domain having the vhCDR1-3 of any of the STEAP1 binding domain variable heavy domains in FIG. 22 or FIG. 31; and b) a variable light domain having the vlCDR1-3 of any of the STEAP1 binding domain variable light domains in FIG. 22 or FIG. 32.

In some embodiments, the STEAP1 ABD includes a) a variable heavy domain having at least 85% sequence identity to a STEAP1 binding domain variable heavy domain in FIG. 22 or FIG. 31; and b) a variable light domain having at least 85% sequence identity to a STEAP1 binding domain variable light domain in FIG. 22 or FIG. 32. In some embodiments, the variable heavy domain has at least 90% sequence identity to a STEAP1 binding domain variable heavy domain in FIG. 22 or FIG. 31; and the variable light domain has at least 90% sequence identity to a STEAP1 binding domain variable light domain in FIG. 22 or FIG. 32. In some embodiments, the variable heavy domain has at least 95% sequence identity to a STEAP1 binding domain variable heavy domain in FIG. 22 or FIG. 31; and the variable light domain has at least 95% sequence identity to a STEAP1 binding domain variable light domain in FIG. 22 or FIG. 32. In some embodiments, the variable heavy domain has at least 99% sequence identity to a STEAP1 binding domain variable heavy domain in FIG. 22 or FIG. 31; and the variable light domain has at least 99% sequence identity to a STEAP1 binding domain variable light domain in FIG. 22 or FIG. 32. In some embodiments, the variable heavy domain has an amino acid sequence of a STEAP1 binding domain variable heavy domain in FIG. 22 or FIG. 31; and the variable light domain has an amino acid sequence of a STEAP1 binding domain variable light domain in FIG. 22 or FIG. 32.

In some embodiments, the STEAP1 ABD comprises a STEAP1 binding domain variable heavy domain and a STEAP1 binding domain variable light domain selected from the following: H1 and L1, H1.1 and L1, H1.2 and L1, H1.3 and L1, H1.4 and L1, H1.5 and L1, H1.6 and L1, H1.7 and L1, H1.8 and L1, H1.9 and L1, H1.10 and L1, H1.I1 and L1, H1.12 and L1, H1.13 and L1, H1.14 and L1, H1.15 and L1, H1.16 and L1, H1.17 and L1, H1.18 and L1, H1.19 and L1, H1.20 and L1, H1.21 and L1, H1.22 and L1, H1.23 and L1, H1.24 and L1, H1.25 and L1, H1.26 and L1, H1.27 and L1, H1.28 and L1, H1.29 and L1, H1.30 and L1, H1.31 and L1, H1.32 and L1, H1.33 and L1, H1.34 and L1, H1.35 and L1, H1.36 and L1, H1.37 and L1, H1.38 and L1, H1 and L1.1, H1 and L1.2, H1 and L1.3, H0 and L0, H1.21 and L1.3, H1.34 and L1.3, H1.39 and L1, H1.40 and L1, H1.4I and L1, H1.42 and L1, H1.43 and L1, H1.44 and L1, H1.45 and L1, H1.46 and L1, H1.47 and L1, H1.48 and L1, H1.49 and L1, H1.50 and L1, H1.51 and L1, H1.52 and L1, H1.53 and L1, H1.54 and L1, H1.55 and L1, H1.56 and L1, H1.57 and L1, H1.58 and L1, H1.59 and L1, H1.60 and L1, H1.61 and L1, H1.62 and L1, H1.63 and L1, H1.64 and L1, H1.65 and L1, H1.66 and L1, H1.67 and L1, H1.68 and L1, H1.69 and L1, H1.70 and L1, H1.71 and L1, H1.72 and L1, H1.73 and L1, H1.74 and L1, H1.75 and L1, H1.76 and L1, H1.77 and L1, H1.78 and L1, H1.79 and L1, H1.80 and L1, H1.81 and L1, H1.82 and L1, H1.83 and L1, H1.84 and L1, H1.85 and L1, H1.86 and L1, H1.87 and L1, H1.88, and L1, H1.89 and L1, H1.90 and L1, H1.91 and L1, H1.92 and L1, H1.93 and L1, H1.94 and L1, H1.95 and L1, H1.96 and L1, H1.97 and L1, H1.98 and L1, H1.99 and L1, H1.100 and L1, H1.101 and L1, H1.102 and L1, H1.103 and L1, H1.104 and L1, H1.105 and L1, H1.106 and L1, H1.107 and L1, H1.108 and L1, H1.109 and L1, H1.110 and L1, H1.111 and L1, H1.112 and L1, H1.113 and L1, H1.114 and L1, H1.115 and L1, H1.116 and L1, H1.117 and L1, H1.118 and L1, H1.119 and L1, H1.120 and L1, H1.121 and L1, H1.122 and L1, H1.123 and L1, H1.124 and L1, H1.125 and L1, H1.126 and L1, H1.127 and L1, H1.128 and L1, H1.129 and L1, H1.130 and L1, H1.131 and L1, H1.132 and L1, H1.133 and L1, H1.134 and L1, H1.135 and L1, H1.136 and L1, H1.137 and L1, H1.138 and L1, H1.139 and L1, H1.140 and L1, H1.141 and L1, H1.142 and L1, H1.143 and L1, H1.144 and L1, H1.145 and L1, H1.146 and L1, H1.147 and L1, H1.148 and L1, H1.149 and L1, H1.150 and L1, H1.151 and L1, H1.152 and L1, H1.153 and L1, H1.154 and L1, H1.155 and L1, H1.156 and L1, H1.157 and L1, H1.158 and L1, H1.159 and L1, H1.160 and L1, H1.161 and L1, H1.162 and L1, H1.163 and L1, H1.164 and L1, H1.165 and L1, H1.166 and L1, H1.167 and L1, H1.168 and L1, H1.169 and L1, H1.170 and L1, H1.171 and L1, H1.172 and L1, H1.173 and L1, H1.174 and L1, H1.175 and L1, H1.176 and L1, H1.177 and L1, H1.178 and L1, H1.179 and L1, H1.180 and L1, H1.181 and L1, H1.182 and L1, H1.183 and L1, H1.184 and L1, H1.185 and L1, H1.186 and L1, H1.187 and L1, H1.188 and L1, H1.189 and L1, H1 and L1.4, H1 and L1.5, H1 and L1.6, H1 and L1.7, H1 and L1.8, H1 and L1.9, H1 and L1.10, H1 and L1.11, H1 and L1.12, H1 and L1.13, H1 and L1.14, H1 and L1.15, H1 and L1.16, H1 and L1.17, H1 and L1.18, H1 and L1.19, H1 and L1.20, H1 and L1.21, H1 and L1.22, H1 and L1.23, H1 and L1.24, H1 and L1.25, H1 and L1.26, H1 and L1.27, H1 and L1.28, H1 and L1.29, H1 and L1.30, H1 and L1.31, H1 and L1.32, H1 and L1.33, H1 and L1.34, H1 and L1.35, H1 and L1.36, H1 and L1.37, H1 and L1.38, H1 and L1.39, H1 and L1.40, H1 and L1.41, H1 and L1.42, H1 and L1.43, H1 and L1.44, H1 and L1.45, H1 and L1.46, H1 and L1.47, H1 and L1.48, H1 and L1.49, H1 and L1.50, H1 and L1.51, H1 and L1.52, H1 and L1.53, H1 and L1.54, H1 and L1.55, H1 and L1.56, H1 and L1.57, H1 and L1.58, H1 and L1.59, H1 and L1.60, H1 and L1.61, H1 and L1.62, H1 and L1.63, H1 and L1.64, H1 and L1.65, H1 and L1.66, H1 and L1.67, H1 and L1.68, H1 and L1.69, H1 and L1.70, H1 and L1.71, H1 and L1.72, H1 and L1.73, H1 and L1.74, H1 and L1.75, H1 and L1.76, H1 and L1.77, H1 and L1.78, H1 and L1.79, H1 and L1.80, H1 and L1.81, H1 and L1.82, H1 and L1.83, H1 and L1.84, H1 and L1.85, H1.42 and L1.47, H1.42 and L1.56, H1.190 and L1.3, H1.191 and L1.3, H1.192 and L1.3, H1.195 and L1.3, H1.196 and L1.3, H1.197 and L1.3, H1.198 and L1.3, H1.199 and L1.3, H1.200 and L1.3, H1.201 and L1.3, H1.202 and L1.3, H1.203 and L1.3, H1.204 and L1.3, H1.205 and L1.3, H1.206 and L1.3, H1.207 and L1.3, H1.208 and L1.3, H1.209 and L1.3, H1.210 and L1.3, H1.211 and L1.3, H1.212 and L1.3, H1.213 and L1.3, H1.214 and L1.3, H1.215 and L1.3, H1.216 and L1.3, H1.217 and L1.3, H1.218 and L1.3, H1.219 and L1.3, H1.220 and L1.3, H1.221 and L1.3, H1.222 and L1.3, H1.223 and L1.3, H1.224 and L1.3, H1.225 and L1.3, H1.226 and L1.3, H1.227 and L1.3, H1.228 and L1.3, H1.21 and L1.92, H1.21 and L1.93, H1.21 and L1.94, H1.21 and L1.95, H1.21 and L1.96, H1.21 and L1.97, H1.21 and L1.98, H1.21 and L1.99, H1.21 and L1.100, H1.21 and L1.101, H1.21 and L1.102, H1.21 and L1.103, H1.21 and L1.104, H1.21 and L1.105, H1.21 and L1.106, H1.21 and L1.107, H1.21 and L1.108, H1.21 and L1.109, H1.21 and L1.110, H1.21 and L1.111, H1.21 and L1.112, H1.21 and L1.113, H1.21 and L1.114, H1.21 and L1.115, H1.21 and L1.116, H1.21 and L1.117, H1.21 and L1.118, H1.21 and L1.119, H1.195 and L1.92, H1.195 and L1.94, H1.195 and L1.95, H1.195 and L1.104, H1.195 and L1.105, H1.195 and L1.109, H1.196 and L1.92, H1.196 and L1.94, H1.196 and L1.95, H1.196 and L1.104, H1.196 and L1.105, H1.196 and L1.109, H1.205 and L1.92, H1.205 and L1.94, H1.205 and L1.95, H1.205 and L1.104, H1.205 and L1.105, H1.205 and L1.109, H1.193 and L1.91, H1.194 and L1.86, H1.194 and L1.87, H1.194 and L1.88, H1.194 and L1.89, or H1.194 and L1.90, or H1.2320 and L1.100, or H1.230 and L1.115 (FIGS. 22, 31, 32 and 39).

In one aspect, provided herein is an anti-CD28 x anti-CEACAM5 heterodimeric antibody that comprises a) a first monomer; b) a second monomer; and c) a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 and the VL1 together form a first antigen binding domain (ABD) and the VH2 and the VL2 together form a second ABD. Further, one of the first ABD and second ABD is a CD28 binding domain and the other of the first ABD and second ABD is a carcinoembryonic antigen-related cell adhesion molecule 5 (CEACAM5) binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs is the CEACAM5 binding domain and the second ABD is the CD28 binding domain. In some embodiments, VH1 and VL1 are a selected from the following VH and VL of any of the CEACAM5 binding domains from FIG. 41 and FIG. 45 or a variant thereof. In some embodiments, VH2 and VL2 are selected from one of the following: 1) a VH and VL of any of the CD28 binding domains from FIGS. 15, 18, 21 and 65 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is an anti-CD28 x anti-CEACAM5 heterodimeric antibody that comprises a) a first monomer; b) a second monomer; c) a first light chain; and d) a second light chain. The first monomer comprises, from N-terminal to C-terminal, VH1-CH1-first domain linker-scFv-second domain linker-CH2-CH3, wherein VH1 is a first variable heavy domain, and CH2-CH3 is a first Fc domain. The second monomer comprises, from N-terminal to C-terminal, a VH1-CHT-hinge-CH2-CH3, wherein CH2-CH3 is a second Fc domain. The first light chain comprises, from N-terminal to C-terminal, VL1-CL, wherein VL1 is a first variable light domain and CL is a constant light domain. The second light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2), wherein, the VH1 of the first monomer and the VL1 of the first light chain and the VH1 of the second monomer and the VL1 of the second light chain each form a first antigen binding domain (ABD), and the VH2 and the VL2 form a second ABD. Further, the first ABDs are carcinoembryonic antigen-related cell adhesion molecule 5 (CEACAM5) binding domains and the second ABD is a CD28 binding domain, or the first ABDs are CD28 binding domains and the second ABD is an CEACAM5 binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the CEACAM5 binding domains and the second ABD is the CD28 binding domain. In some embodiments, VH1 and VL1 are selected from the following: 1) a VH and VL of any of the CEACAM5 binding domains from FIG. 41 and FIG. 45 or a variant thereof. In some embodiments, VH2 and VL2 are selected from one of the following: 1) a VH and VL of any of the CD28 binding domains from FIGS. 15, 18, 21 and 65 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering

In certain embodiments, the first and second Fe domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is an anti-CD28 x anti-CEACAM5 heterodimeric antibody that comprises a) a first monomer; b) a second monomer; and c) a first light chain; and d) a second light chain. The first monomer comprises, from N-terminal to C-terminal, VH1-CHT-hinge-CH2-CH3-domain linker-scFv, wherein VH1 is a first variable heavy domain, and CH2-CH3 is a first Fc domain. The second monomer comprises, from N-terminal to C-terminal, a VH1-CHT-hinge-CH2-CH3, wherein VH1 is a first variable heavy domain and CH2-CH3 is a second Fc domain. The first light chain comprises, from N-terminal to C-terminal, VL1-CL, wherein VL1 is a first variable light domain and CL is a constant light domain. The second light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2), wherein the VH1 of the first monomer and the VL1 of the first light chain and the VH1 of the second monomer and the VL1 of the second light chain each form a first antigen binding domain (ABD), and the VH2 and the VL2 form a second ABD. Further, the first ABDs are carcinoembryonic antigen-related cell adhesion molecule 5 (CEACAM5) binding domains and the second ABD is a CD28 binding domain, or the first ABDs are CD28 binding domains and the second ABD is an CEACAM5 binding domain

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the CEACAM5 binding domains and the second ABD is the CD28 binding domain. In some embodiments, VH1 and VL1 are selected from the following: 1) a VH and VL of any of the CEACAM5 binding domains from FIG. 41 and FIG. 45 or a variant thereof. In some embodiments, VH2 and VL2 are selected from one of the following: 1) a VH and VL of any of the CD28 binding domains from FIGS. 15, 18, 21 and 65 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fc domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is a heterodimeric antibody that comprises a) a first monomer, b) a second monomer, c) a first light chain, and d) a second light chain. The first monomer comprises, from N-terminal to C-terminal, a VH1-CH1-domain linker-VH1-CH1-hinge-CH2-CH3, wherein each of the VHls is a first variable heavy domain, and CH2-CH3 is a first Fc domain. The second monomer comprises, from N-terminal to C-terminal, an scFv-domain linker-CH2-CH3, wherein VH1 is a first variable heavy domain and CH2-CH3 is a second Fc domain. The first common light chain comprises, from N-terminal to C-terminal, VL1-CL, wherein VL1 is a first variable light domain and CL is a constant light domain. The second light chain comprising, from N-terminal to C-terminal, VL1-CL, wherein VL1 is a first variable light domain and CL is a constant light domain. The scFv comprises a second VH domain (VH2), a scFv linker, and a second variable light domain (VL2), wherein the VH1 of the first monomer and the VL1 of the first light chain and the VH1 of the second monomer and the VL1 of the second light chain each form a first antigen binding domain (ABD), and the VH2 and the VL2 form a second ABD. Further, the first ABDs are carcinoembryonic antigen-related cell adhesion molecule 5 (CEACAM5) binding domains and the second ABD is a CD28 binding domain, or the first ABDs are CD28 binding domains and the second ABD is an CEACAM5 binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the CEACAM5 binding domains and the second ABD is the CD28 binding domain. In some embodiments, VH1 and VL1 are selected from the following: 1) a VH and VL of any of the CEACAM5 binding domains from FIG. 41 and FIG. 45 or a variant thereof. In some embodiments, VH2 and VL2 are selected from one of the following: 1) a VH and VL of any of the CD28 binding domains from FIGS. 15, 18, 21 and 65 or a variant thereof; or 2) (i) a VH from FIG. 15 or 16 or a variant thereof; and (ii) a VL from FIGS. 15, 17 or 82 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering. In some embodiments, the first and second Fe domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421 D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO: 24).

In another aspect, provided herein is a bispecific antibody that comprises: a) a CEACAM5 binding domain comprising i) a first variable heavy domain (VH1), and ii) a first variable light domain (VL1); and b) an anti-CD28 binding domain comprising i) a second variable heavy domain (VH2), and ii) a second variable light domain (VL2). In some embodiments, VH1 and VL1 are selected from the following: 1) a VH and VL of any of the CEACAM5 binding domains from FIG. 41 and FIG. 45 or a variant thereof. In some embodiments, VH2 and VL2 are selected from one of the following: 1) a VH and VL of any of the CD28 binding domains from FIGS. 15, 18, 21 and 65 or a variant thereof; or 2) (i) a VH from FIG. 15 or 16 or a variant thereof; and (ii) a VL from FIGS. 15, 17 or 82 or a variant thereof.

In some embodiments of the bispecific antibodies, the first Fc domain and second Fc domain are each variant Fc domains.

In some embodiments of the bispecific antibodies, the first and second Fc domains comprise a set of heterodimerization skew variants selected from the following heterodimerization variants: S364K/E357Q: L368D/K370S; S364K: L368D/K370S; S364K : L368E/K370S; D401K: T411E/K360E/Q362E; and T366W: T366S/L368A/Y407V, wherein numbering is according to EU numbering. In some embodiments, the first and second Fe domains comprise heterodimerization skew variants S364K/E357Q: L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

Also provided herein are nucleic acid compositions comprising nucleic acids encoding the antibodies described herein, expression vector compositions that include such nucleic acids, host cells for making the antibodies that comprise the expression vector compositions, and methods of making the antibodies.

In another aspect, provided herein is a method of treating a CEACAM5-cancer in a patient in need thereof, comprising administering to the patient according an anti-CD28 x anti-CEACAM5 bispecific antibody as described herein.

In another aspect, provided herein is a method of treating a CEACAM5-associated cancer in a patient in need thereof, comprising administering to the patient an anti-CD28 x anti-CEACAM5 bispecific antibody as described herein; and an anti-CD3 x anti-CEACAM5 bispecific antibody.

In another aspect, provided herein is a CEACAM5 antigen binding domain composition that comprises a) a variable heavy domain; and b) a variable light domain. The variable heavy domain comprises the vhCDR1-3 of any of the CEACAM5 binding domain variable heavy domains in FIG. 45. The variable light domain comprises the vlCDR1-3 of any of the CEACAM5 binding domain variable light domains in FIG. 45. In another aspect, provided herein is a CEACAM5 antigen binding domain composition that comprises a) a variable heavy domain; and b) a variable light domain. The variable heavy domain comprises at least 85% sequence identity to a CEACAM5 binding domain variable heavy domain in FIG. 45. The variable light domain comprises at least 85% sequence identity to a CEACAM5 binding domain variable light domain in FIG. 45.

In some embodiments, the variable heavy domain has at least 90% sequence identity to a CEACAM5 binding domain variable heavy domain in FIG. 45; and the variable light domain has at least 90% sequence identity to a CEACAM5 binding domain variable light domain in FIG. 45.

In some embodiments, the variable heavy domain has at least 95% sequence identity to a CEACAM5 binding domain variable heavy domain in FIG. 45; and the variable light domain has at least 95% sequence identity to a CEACAM5 binding domain variable light domain in FIG. 45.

In some embodiments, the variable heavy domain has at least 99% sequence identity to a CEACAM5 binding domain variable heavy domain in FIG. 45; and the variable light domain has at least 99% sequence identity to a CEACAM5 binding domain variable light domain in FIG. 45.

In some embodiments, the variable heavy domain has an amino acid sequence of a CEACAM5 binding domain variable heavy domain in FIG. 45; and the variable light domain has an amino acid sequence of a CEACAM5 binding domain variable light domain in FIG. 45.

In another aspect, provided herein is a CD28 antigen binding domain composition that comprises a) a variable heavy domain; and b) a variable light domain. The variable heavy domain comprises the vhCDR1-3 of any of the CD28 binding domain variable heavy domains in FIG. 65. The variable light domain comprises the vlCDR1-3 of any of the CD28 binding domain variable light domains in FIG. 65.

In another aspect, provided herein is a CD28 antigen binding domain composition that comprises a) a variable heavy domain; and b) a variable light domain. The variable heavy domain comprises at least 85% sequence identity to a CD28 binding domain variable heavy domain in FIG. 65. The variable light domain comprises at least 85% sequence identity to a CD28 binding domain variable light domain in FIG. 65.

In some embodiments, the variable heavy domain has at least 90% sequence identity to a CD28 binding domain variable heavy domain in FIG. 65; and the variable light domain has at least 90% sequence identity to a CD28 binding domain variable light domain in FIG. 65.

In some embodiments, the variable heavy domain has at least 95% sequence identity to a CD28 binding domain variable heavy domain in FIG. 65; and the variable light domain has at least 95% sequence identity to a CD28 binding domain variable light domain in FIG. 65.

In some embodiments, the variable heavy domain has at least 99% sequence identity to a CD28 binding domain variable heavy domain in FIG. 65; and the variable light domain has at least 99% sequence identity to a CD28 binding domain variable light domain in FIG. 65.

In some embodiments, the variable heavy domain has an amino acid sequence of a CD28 binding domain variable heavy domain in FIG. 65; and the variable light domain has an amino acid sequence of a CD28 binding domain variable light domain in FIG. 65.

In some embodiments, the CD28 binding domain is selected from the following: IA7[CD28]_H1L1, IA7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, 1A7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S]in FIG. 65.

### BRIEF DESCRIPTION OF THE DRAWINGS

Figures iA and 1B depict the sequences for human, mouse, and cynomolgus CD28. Such CD28 are useful for the development of cross-reactive CD28 antigen binding domains for ease of clinical development.

FIGS. 2A and 2B depict STEAP1 and CEACAM5 sequences. FIG. 2A depicts the sequences for human, mouse, and cynomolgus STEAP1 . Such STEAP1 s are useful for the development of cross-reactive STEAP1 antigen binding domains for ease of clinical development. FIG. 2B depicts the sequences for human, mouse, and cynomolgus CEACAM5. Such CEACAM5s are useful for the development of cross-reactive CEACAM5 antigen binding domains for ease of clinical development.

FIG. 3A-3F depict useful pairs of heterodimerization variant sets (including skew and pI variants). In FIG. 3F, there are variants for which there are no corresponding “monomer 2” variants. Such variants are pI variants that can be used alone on either monomer of a bispecific antibody (e.g., STEAP1 x CD28 bsAb or CEACAM5 x CD28 bsAb), or included, for example, on the non-scFv side of a format that utilizes an scFv as a component and an appropriate charged scFv linker can be used on the second monomer that utilizes an scFv as the CD28 binding domain. Suitable charged linkers are shown in FIG. 6. Heterodimer yield (%) and CH3 Tₘ (° C.) of preferred Fc heterodimerization variants were previously described (see, e.g., FIG. 8 of U.S. Patent Application No. 2019/0248898).

FIG. 4 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 5 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 6 depicts a number of charged scFv linkers that find use in increasing or decreasing the pI of the subject heterodimeric bispecific antibodies that utilize one or more scFv as a component, as described herein (e.g., STEAP1 x CD28 and CEACAM5 x CD28 bsAbs). The (+H) positive linker finds particular use herein, particularly with anti-CD28 VL and VH sequences shown herein. A single prior art scFv linker with a single charge is referenced as “Whitlow,” from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIG. 7 depicts a number of exemplary domain linkers. In some embodiments, these linkers find use linking a single-chain Fv to an Fc chain. In some embodiments, these linkers may be combined in any orientation. For example, a GGGGS (SEQ ID NO: 36) linker may be combined with a “lower half hinge” linker at the N-terminus or at the C-terminus.

FIG. 8 shows a particularly useful bispecific antibody platform for the STEAP1 x CD28 and CEACAM5 x CD28 bsAb bsAbs of the invention. Although the platform is described in the context of the 1+1 Fab-scFv-Fc format, it can be adapted for use in other bispecific antibody formats.

FIGS. 9A-9D show the sequences of several useful heterodimeric STEAP1 x CD28 bsAb and CEACAM5 x CD28 bsAb backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236de1/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236de1/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

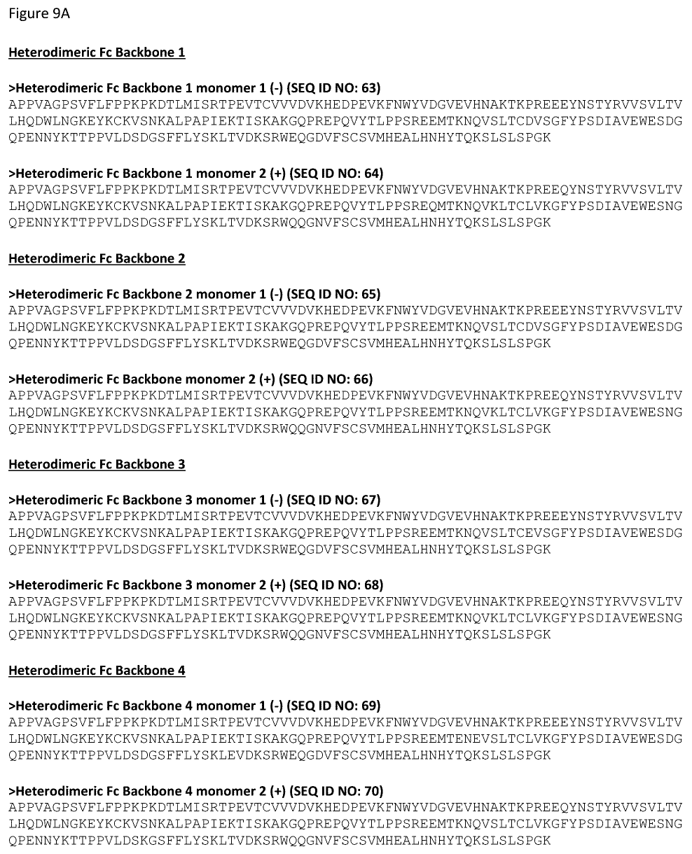

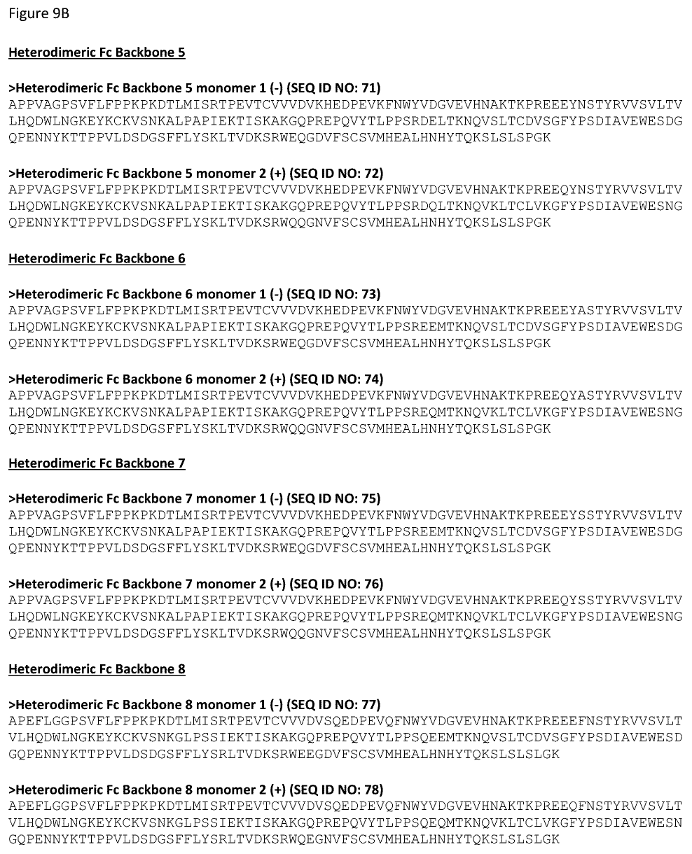

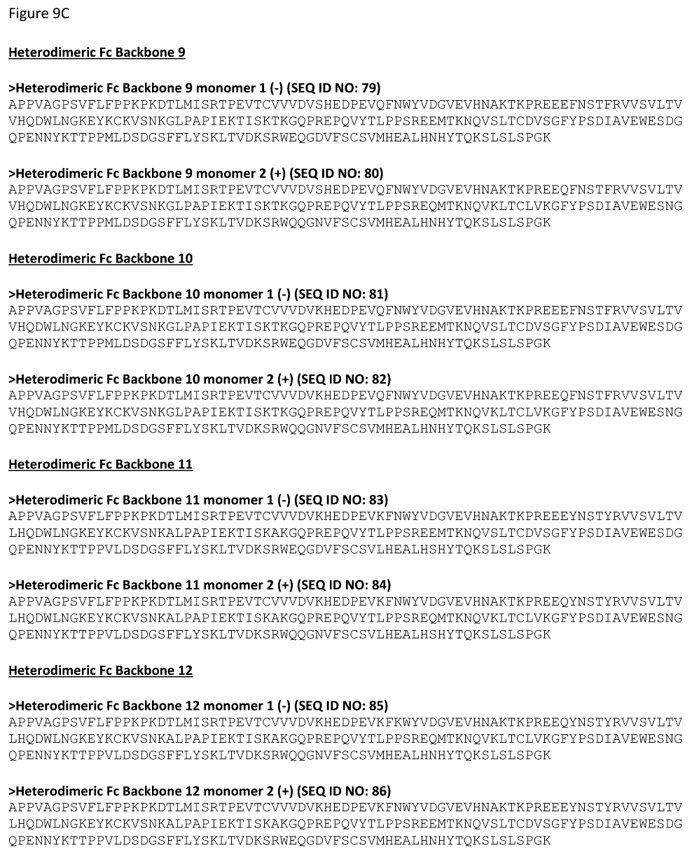

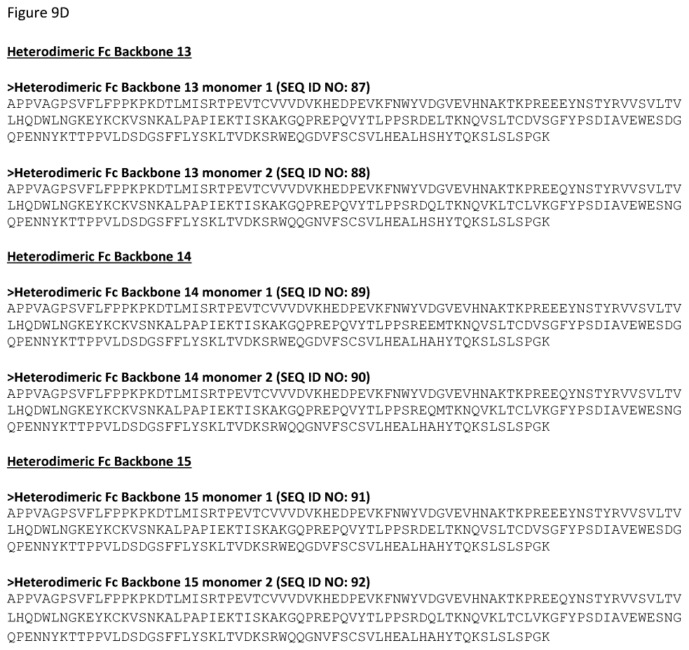

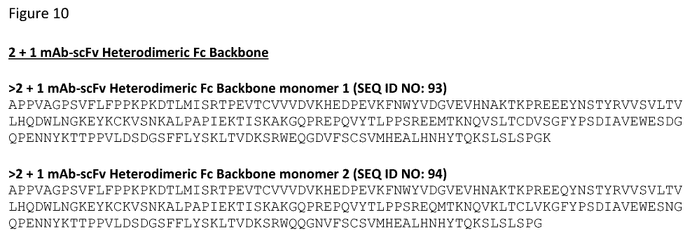

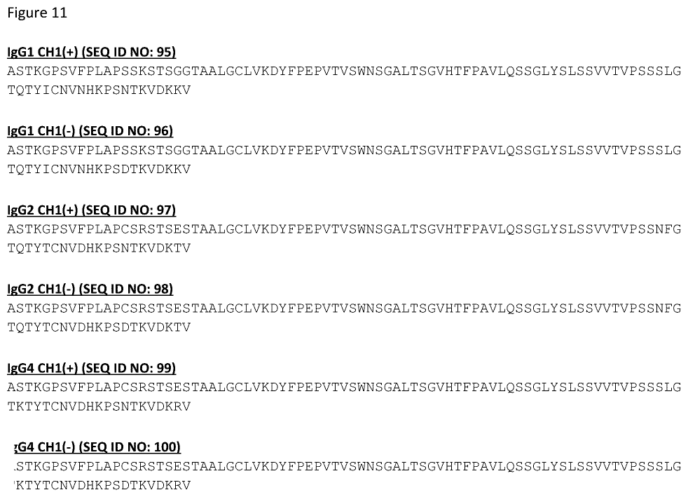

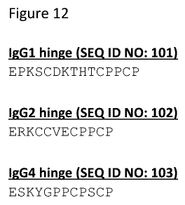

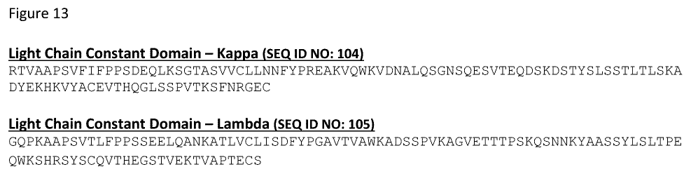

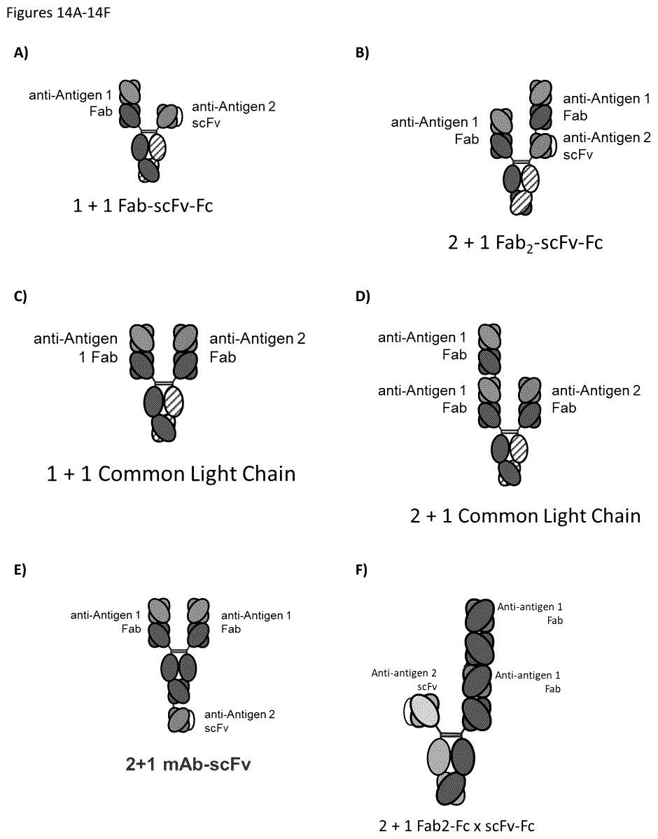

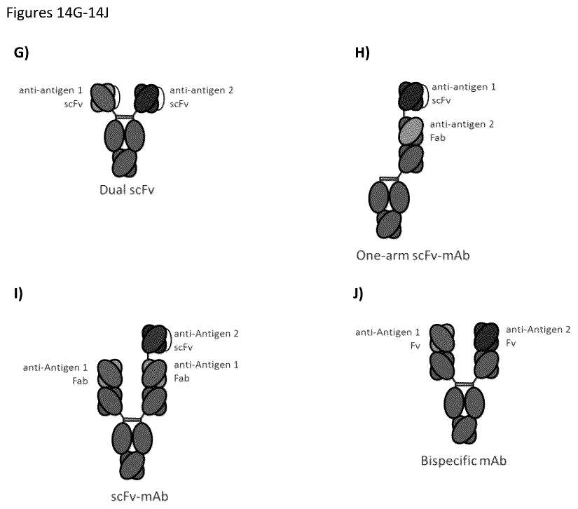

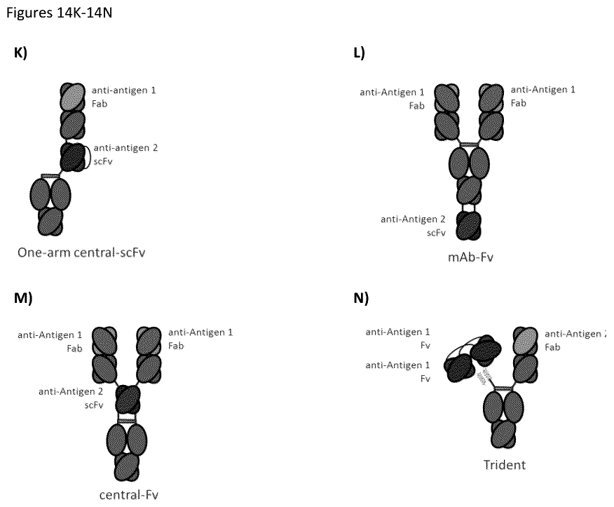

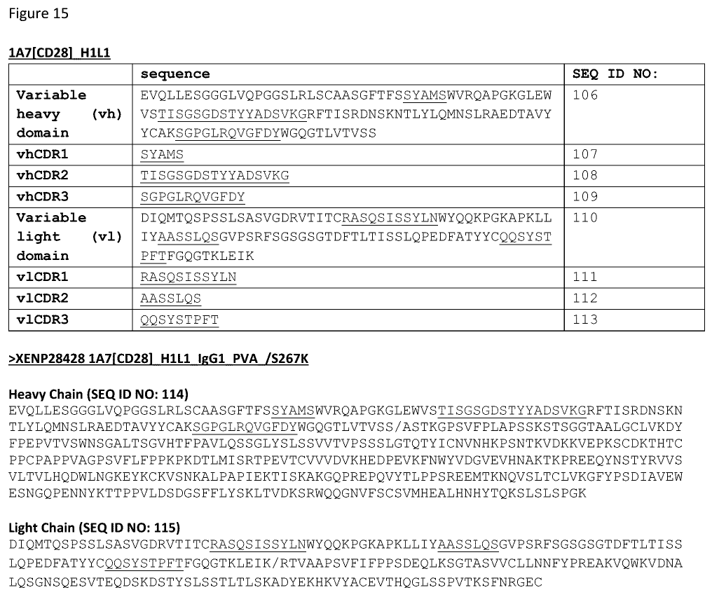

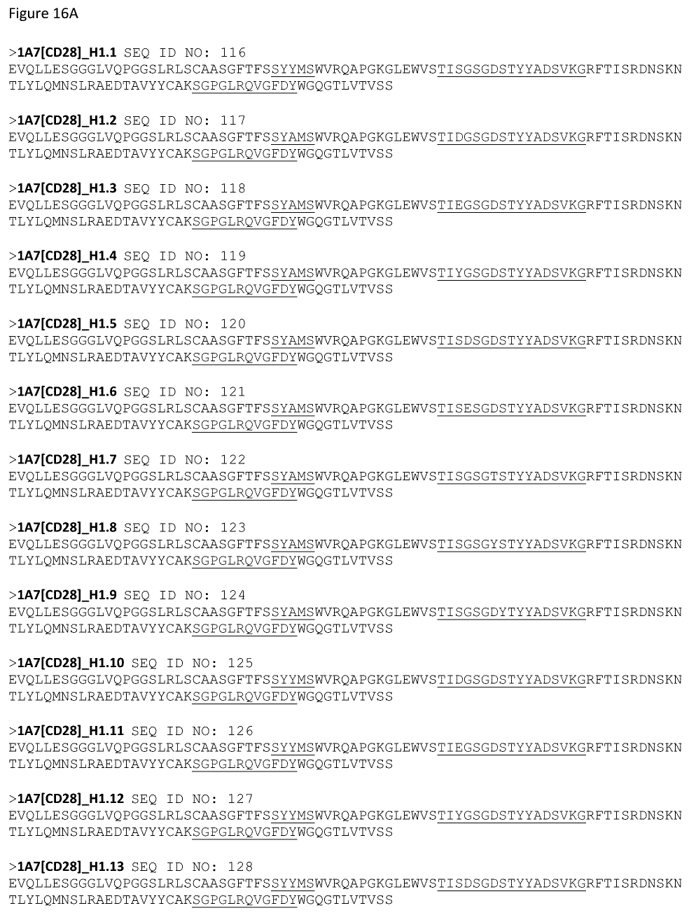

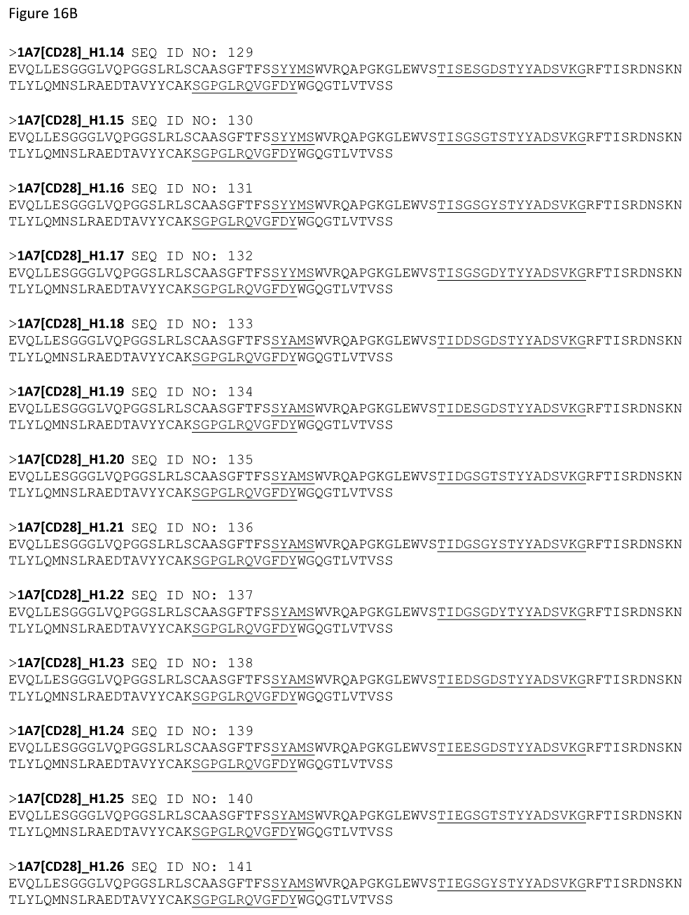

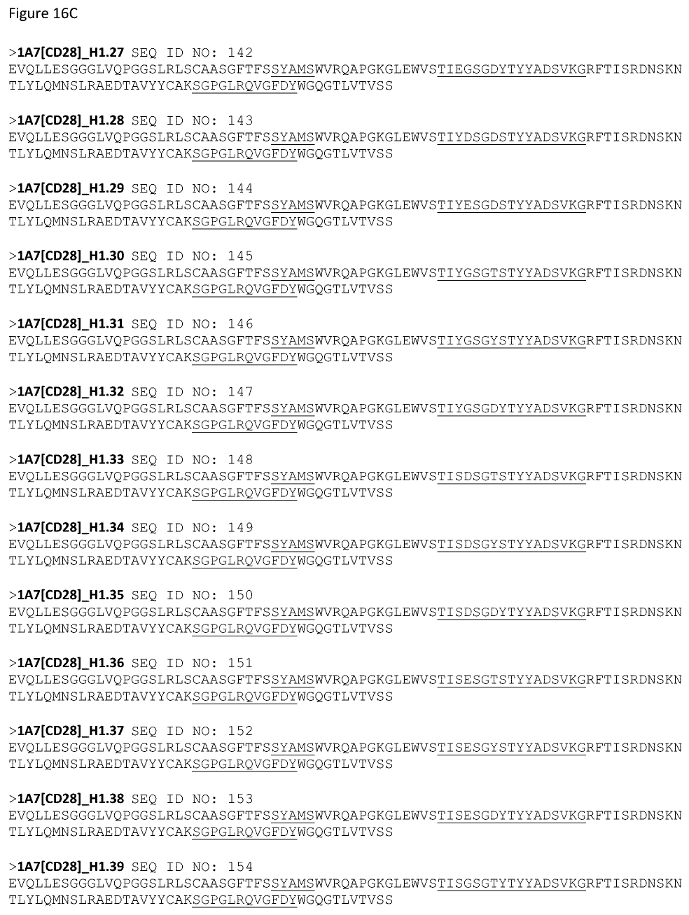

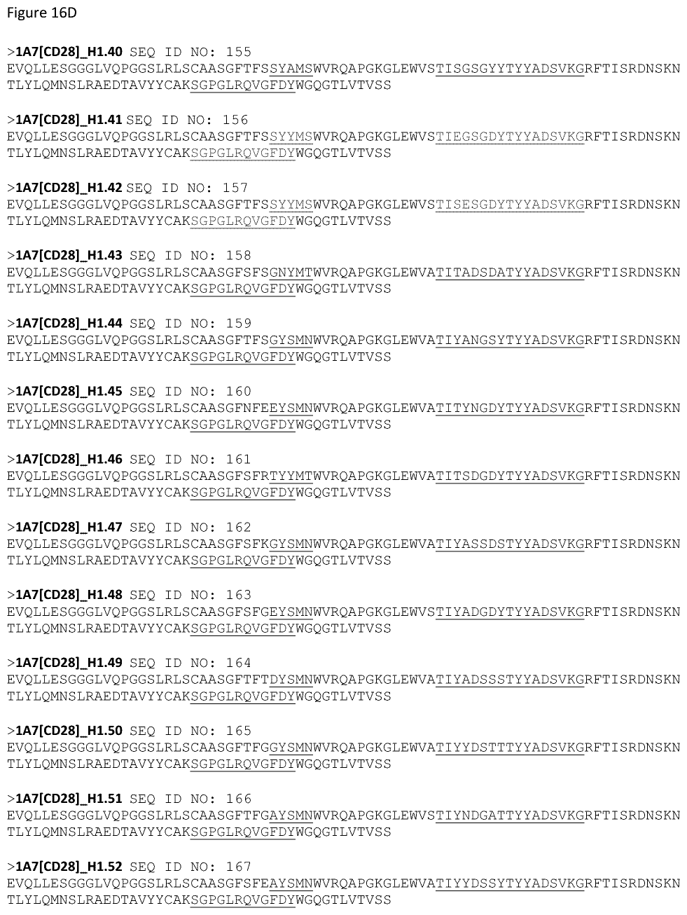

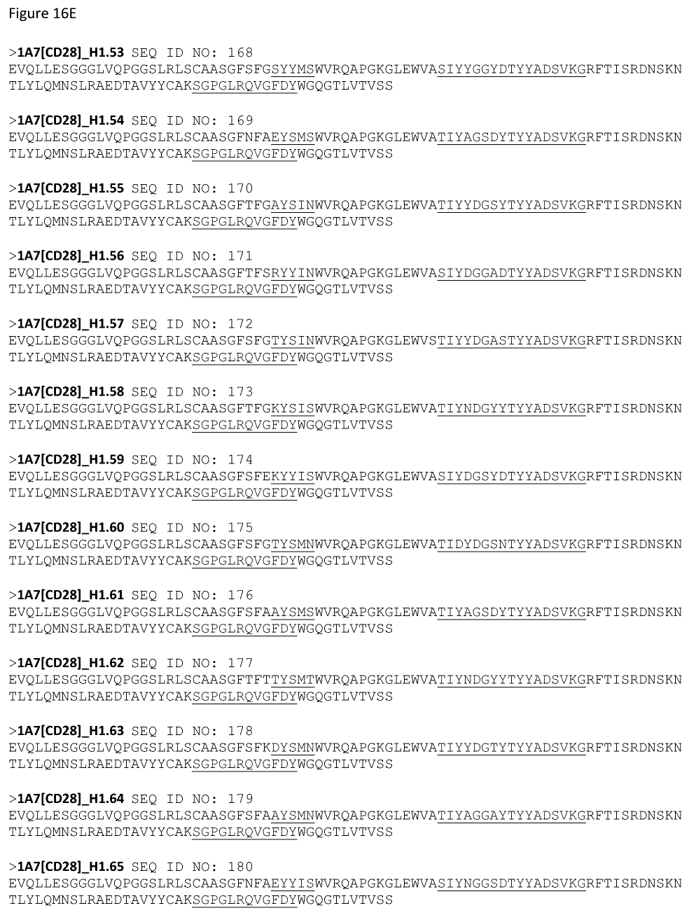

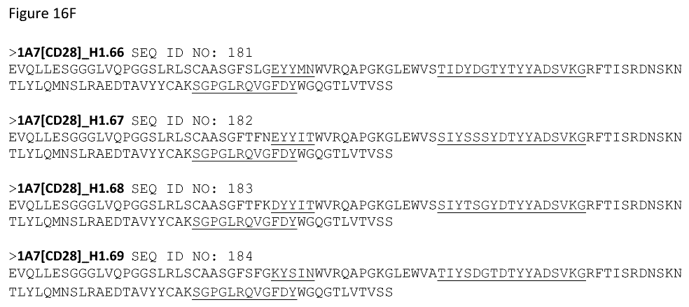

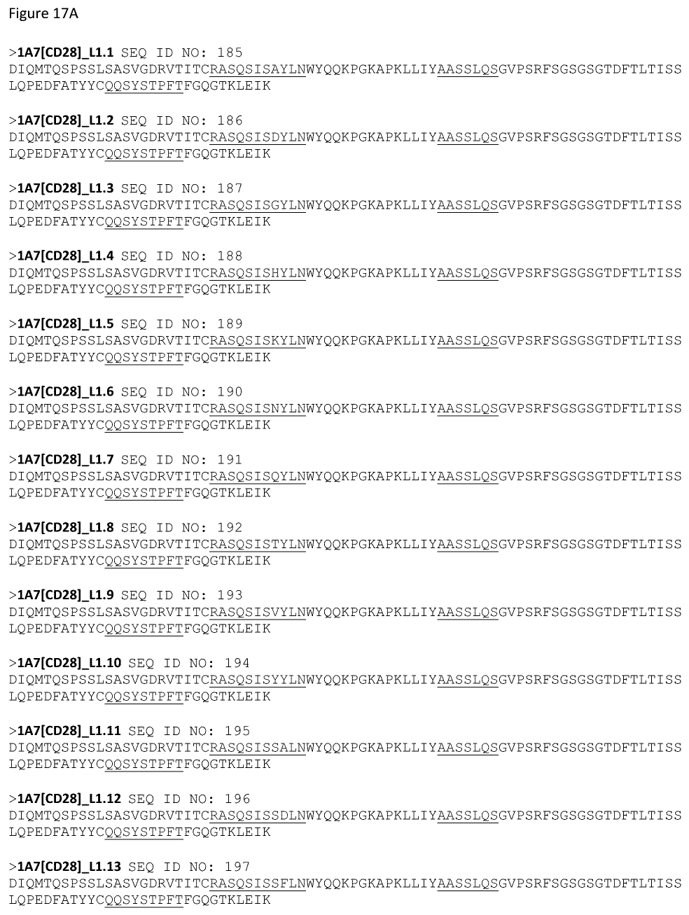

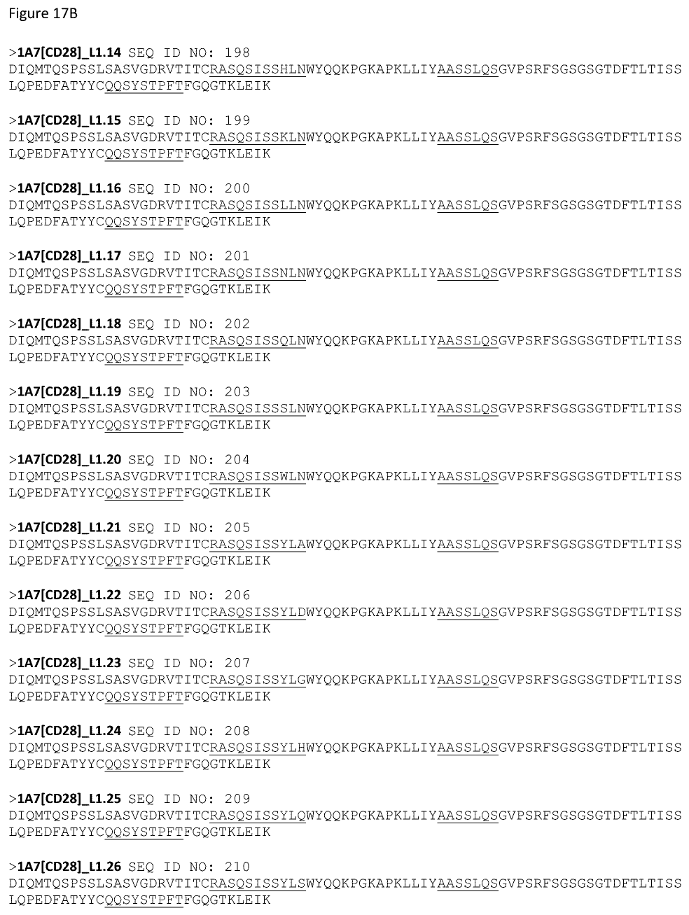

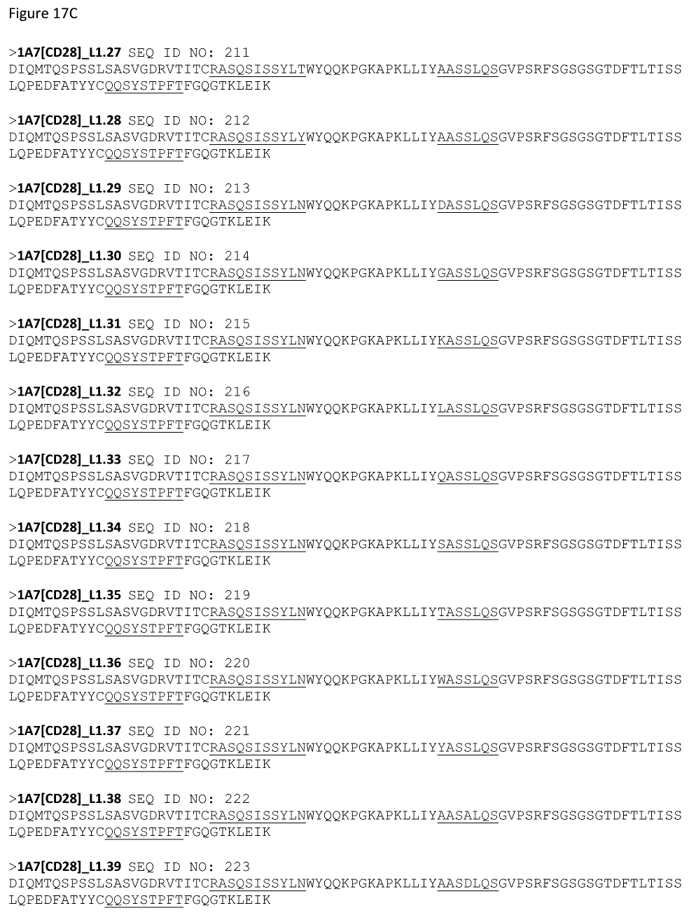

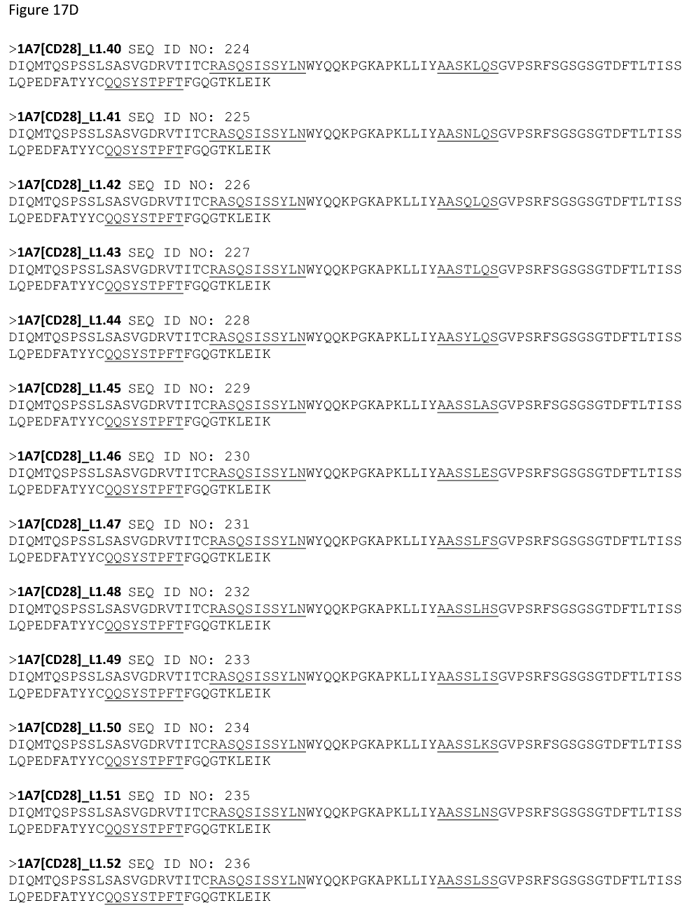

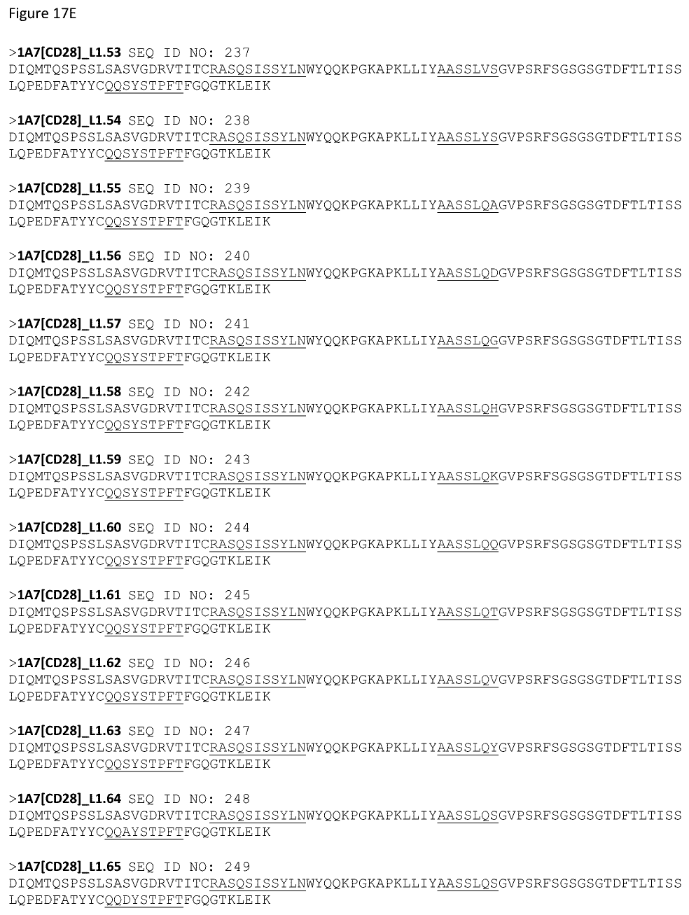

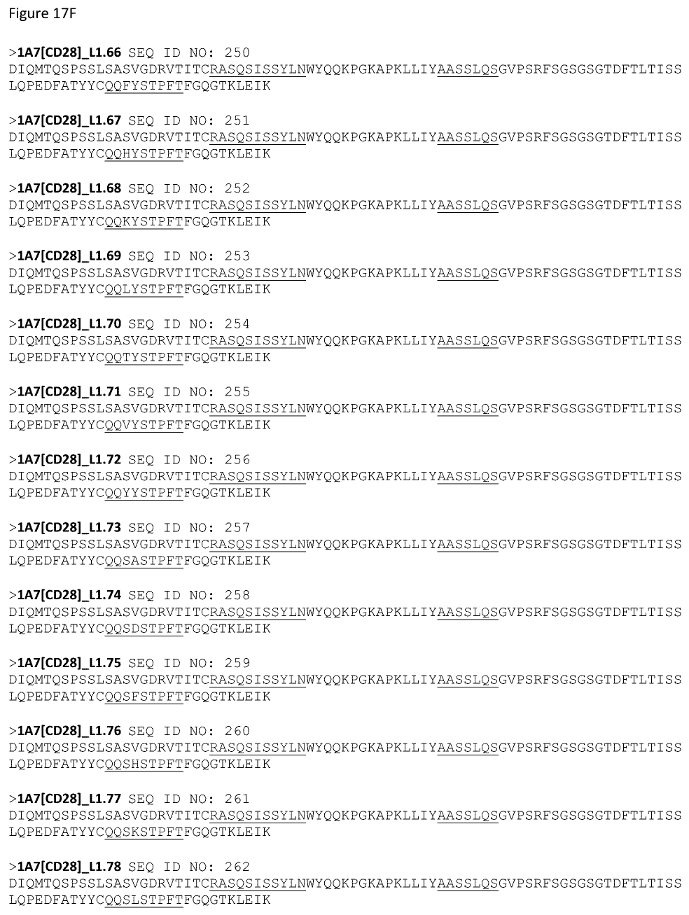

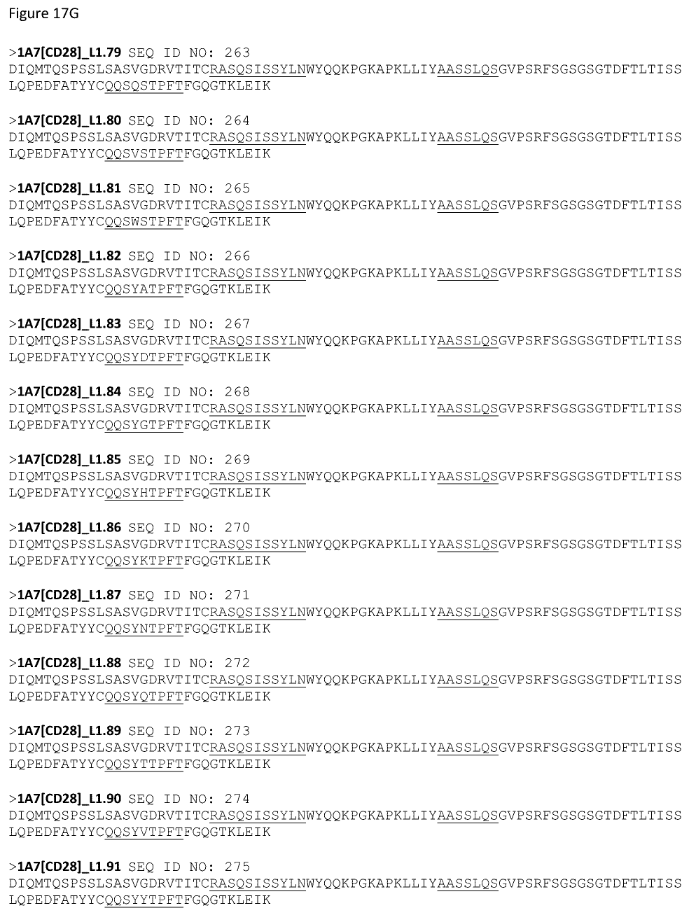

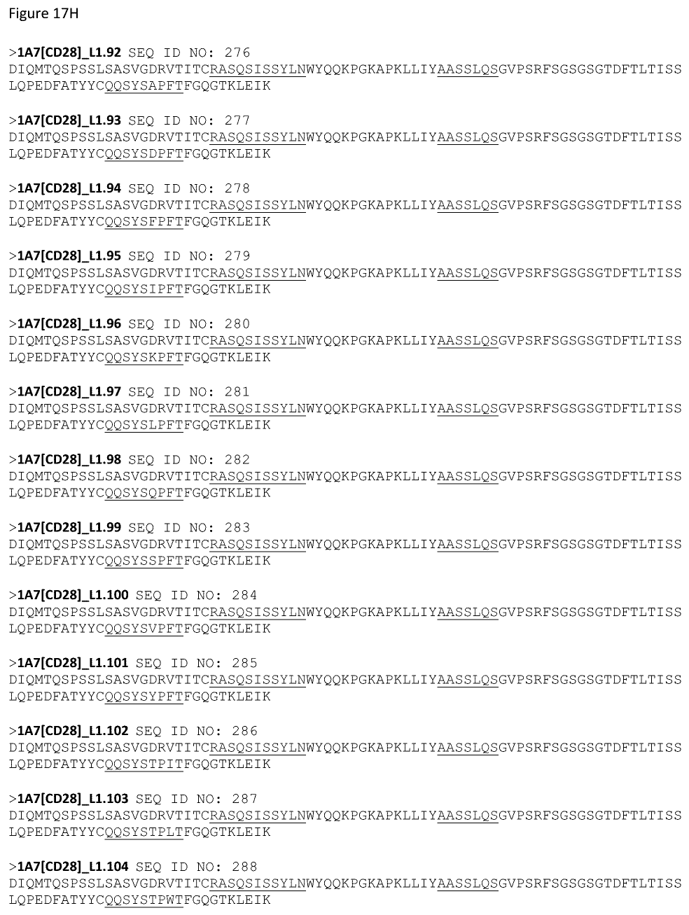

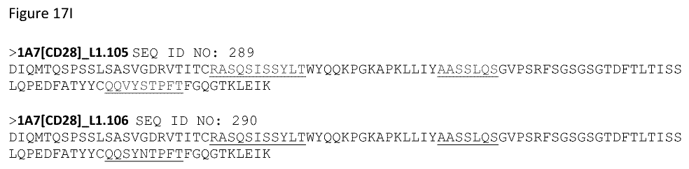

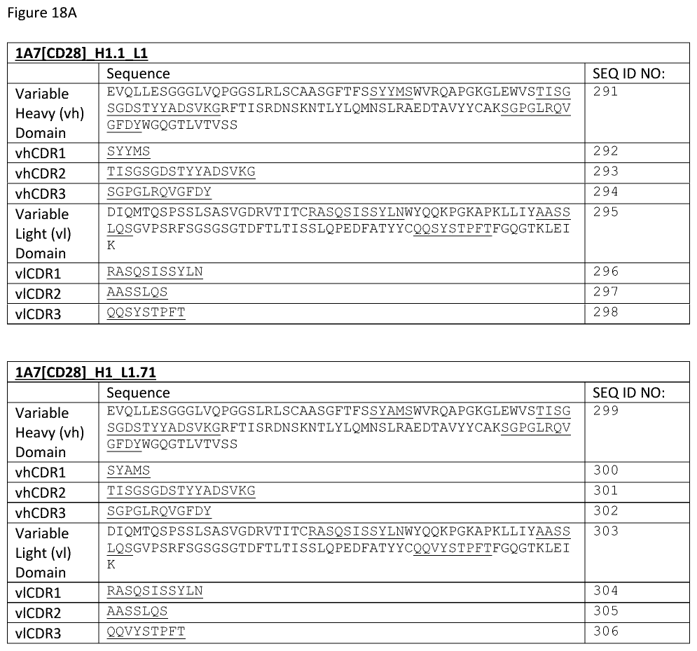

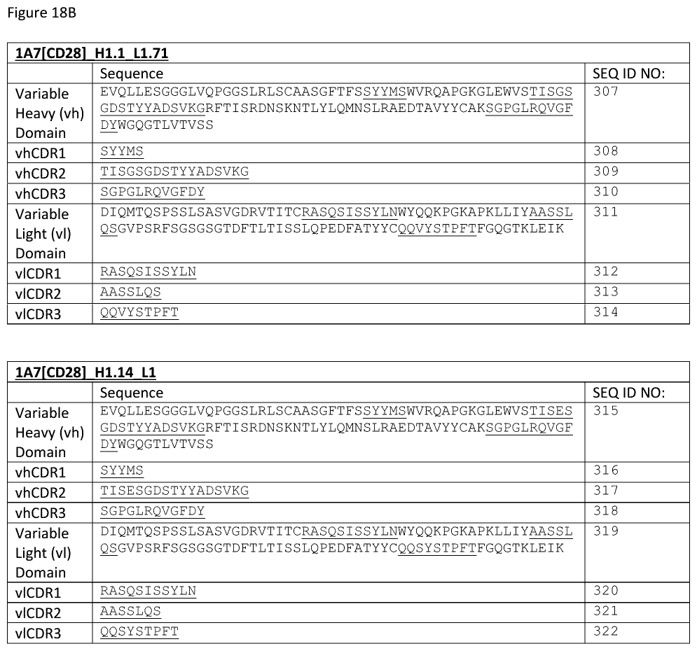

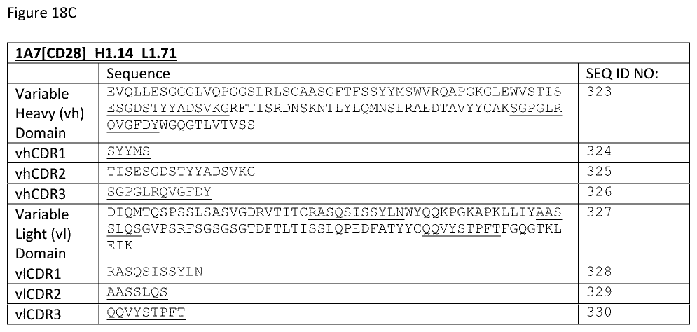

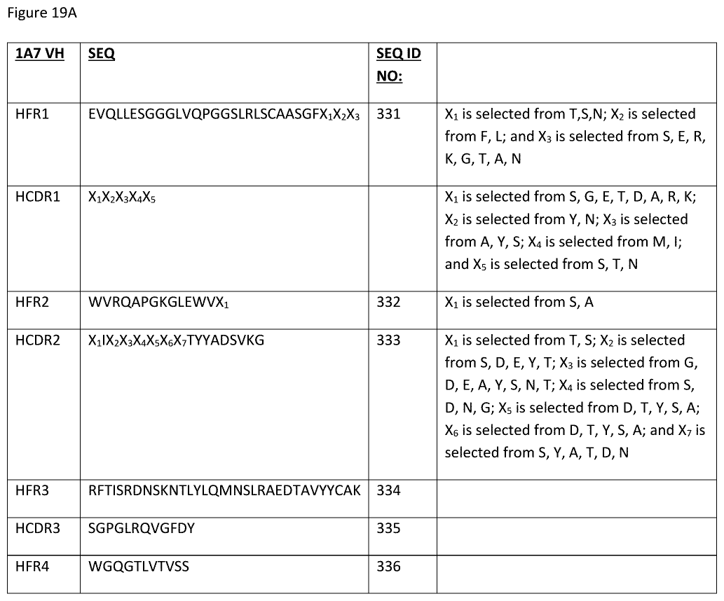

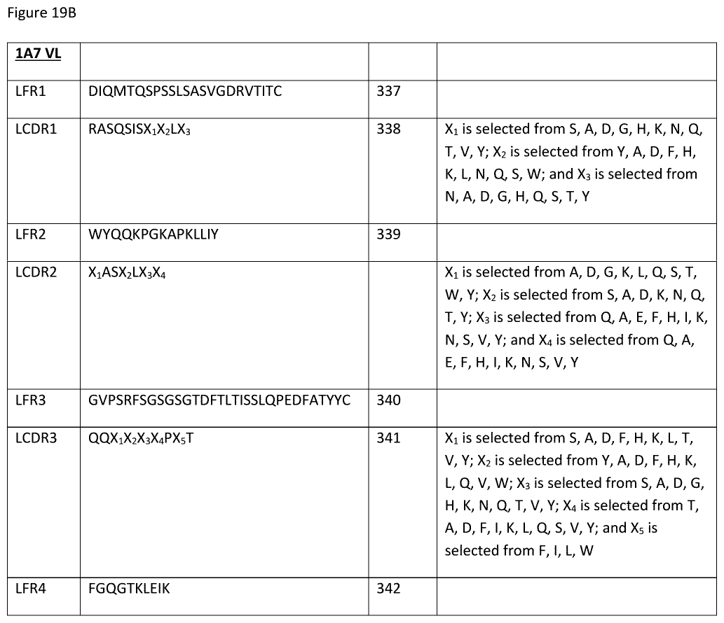

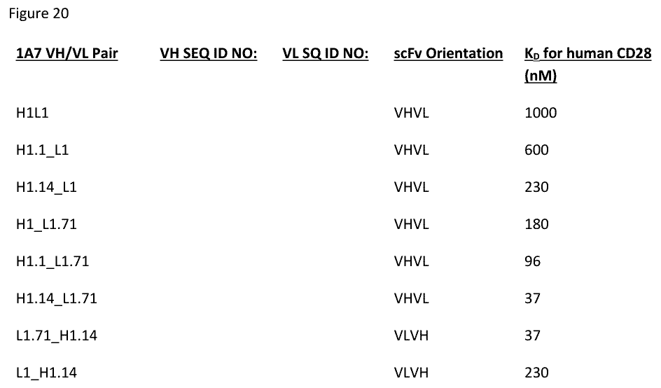

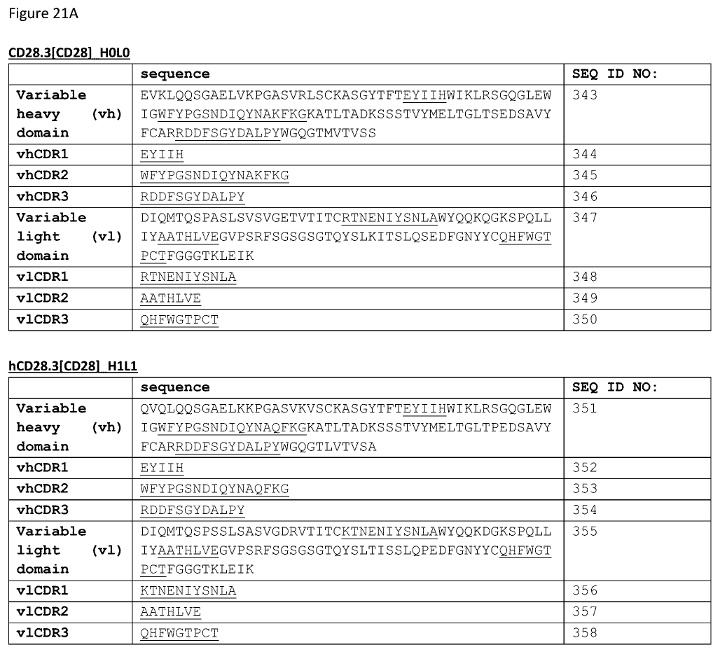

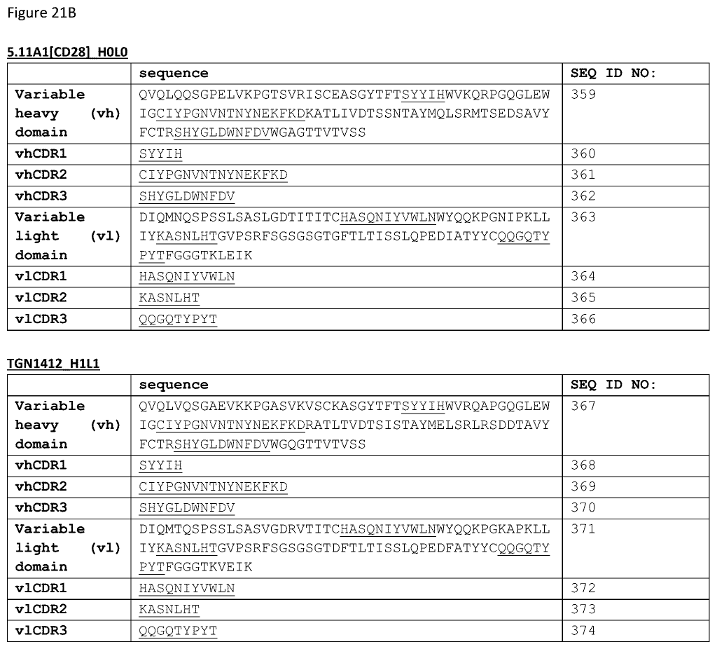

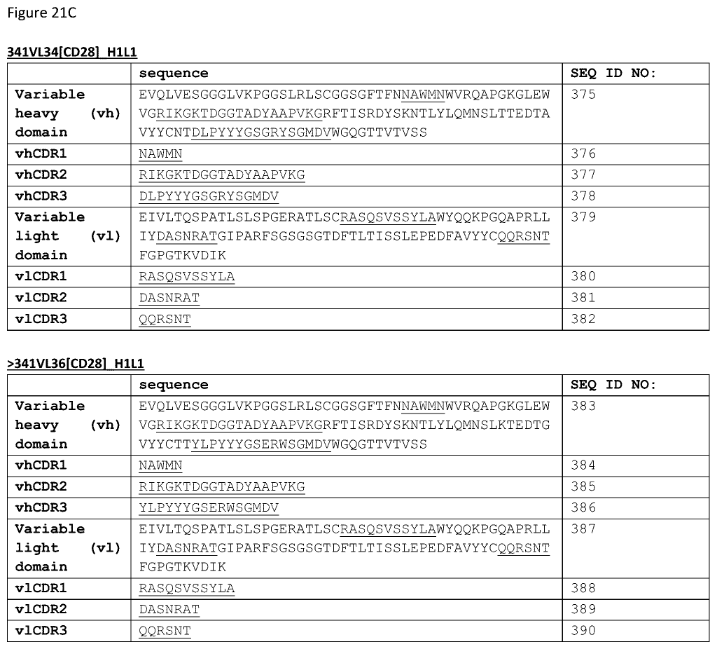

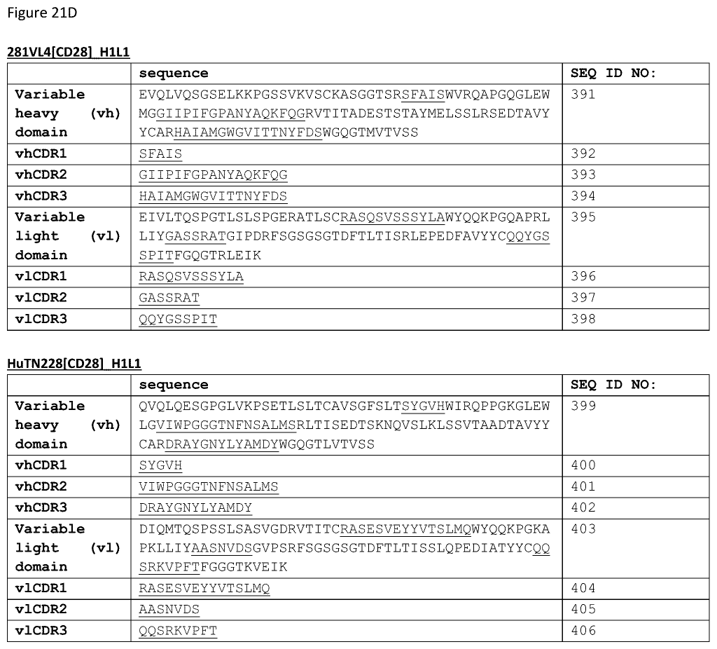

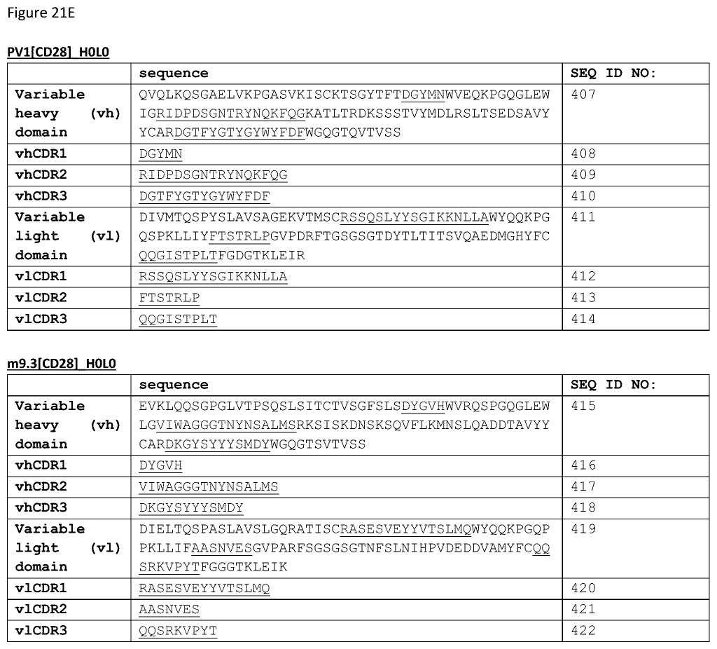

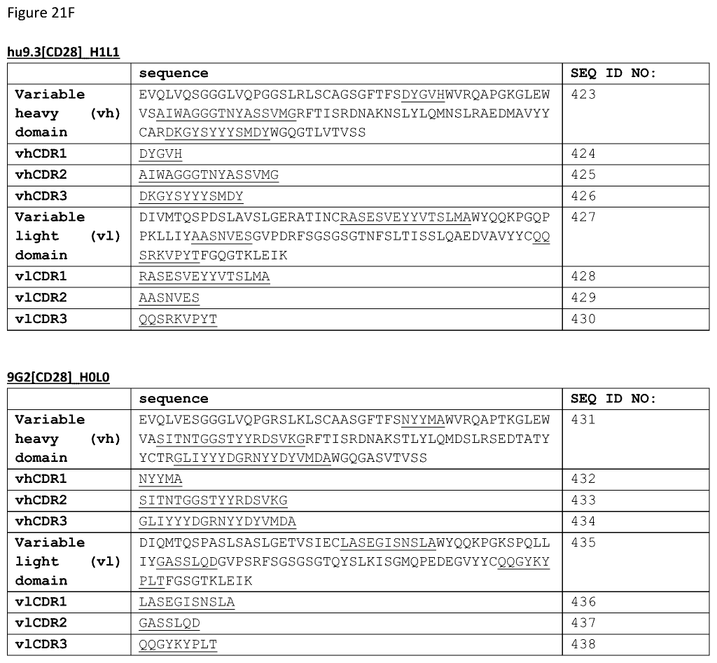

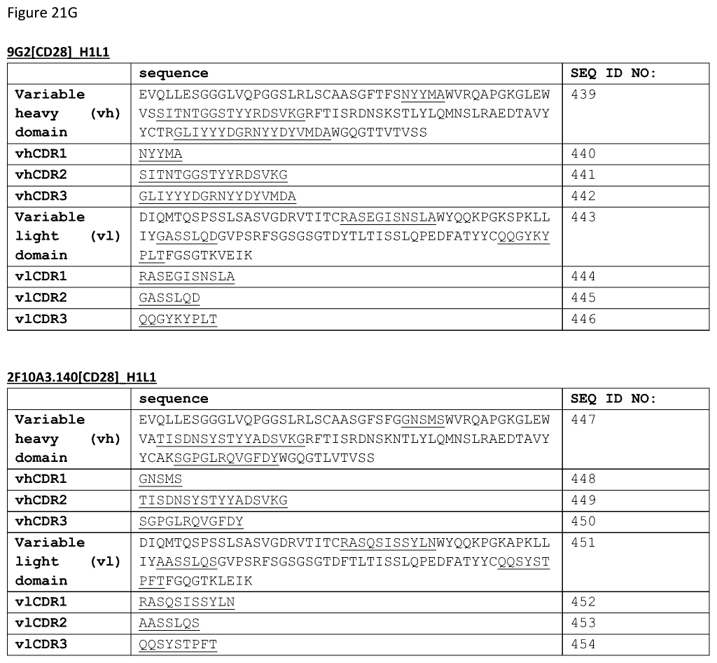

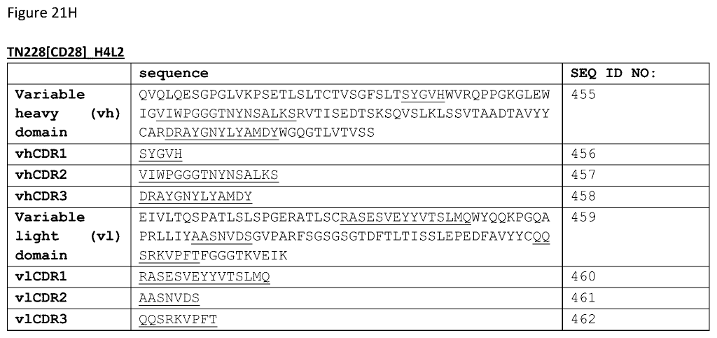

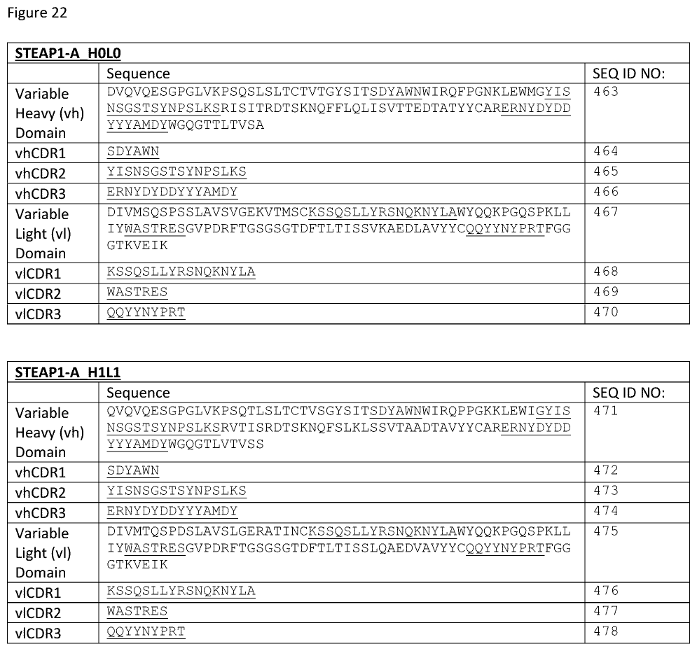

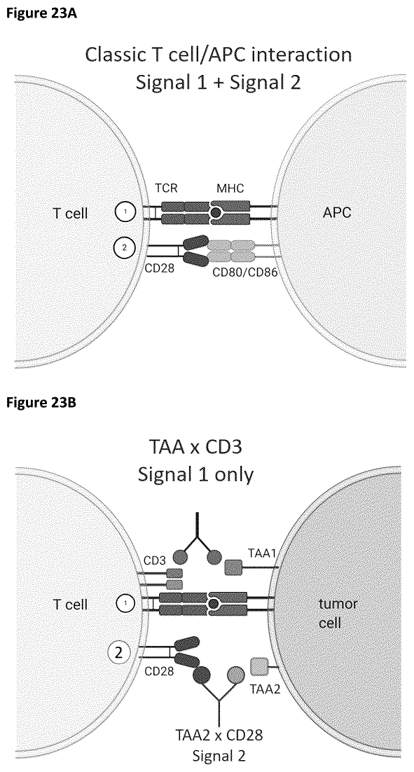

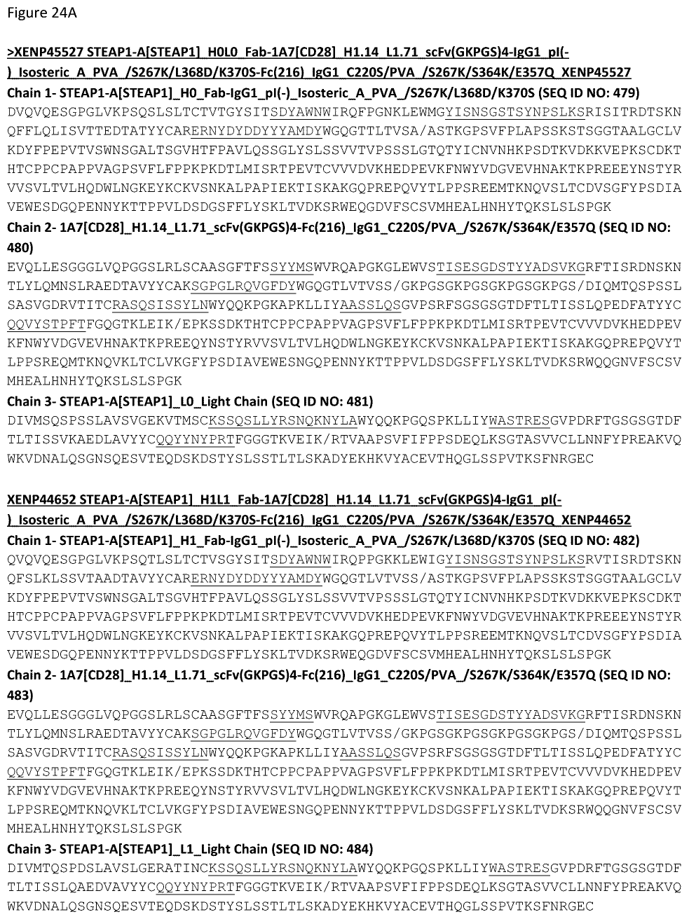

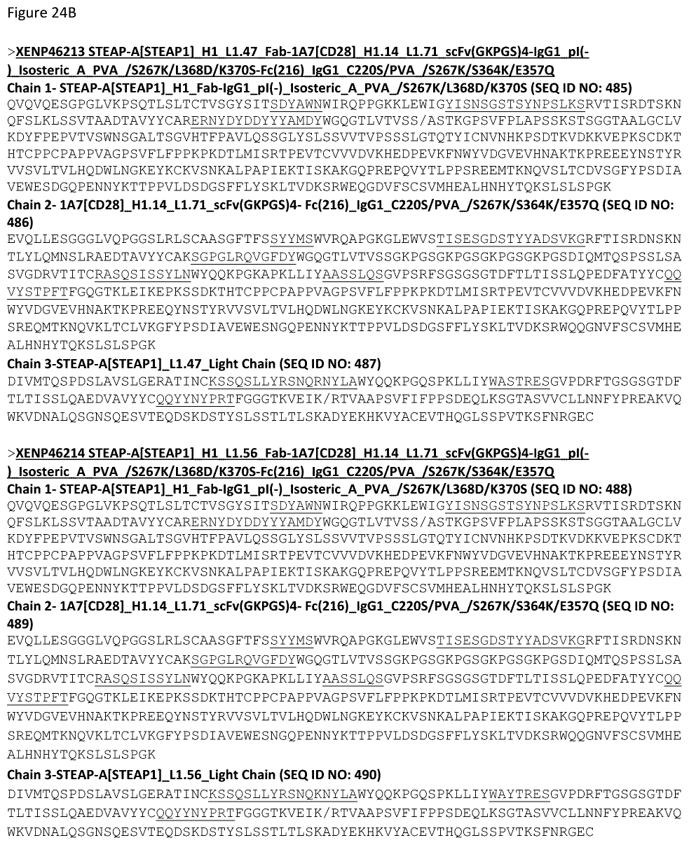

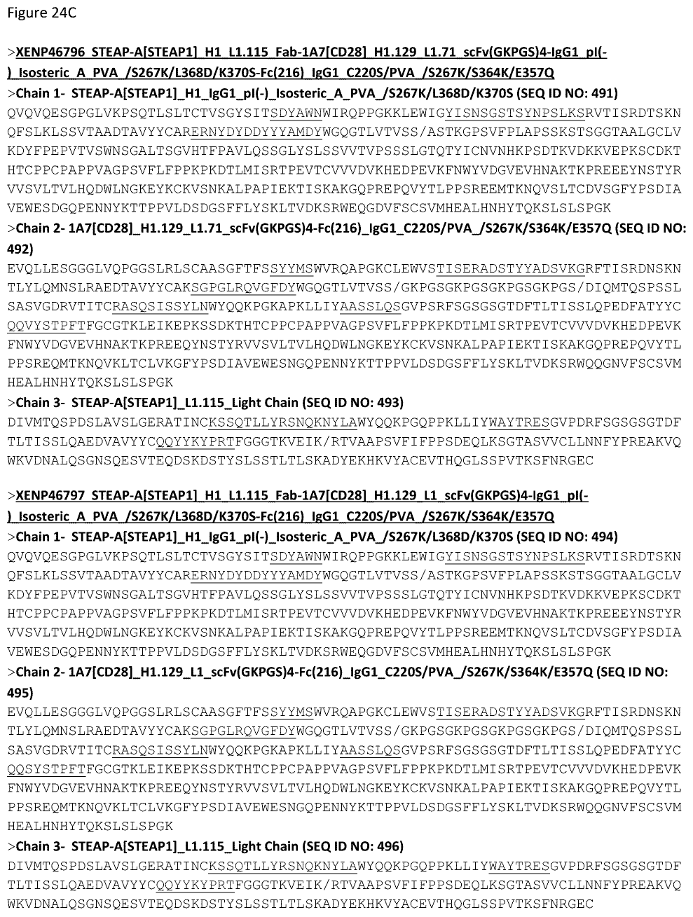

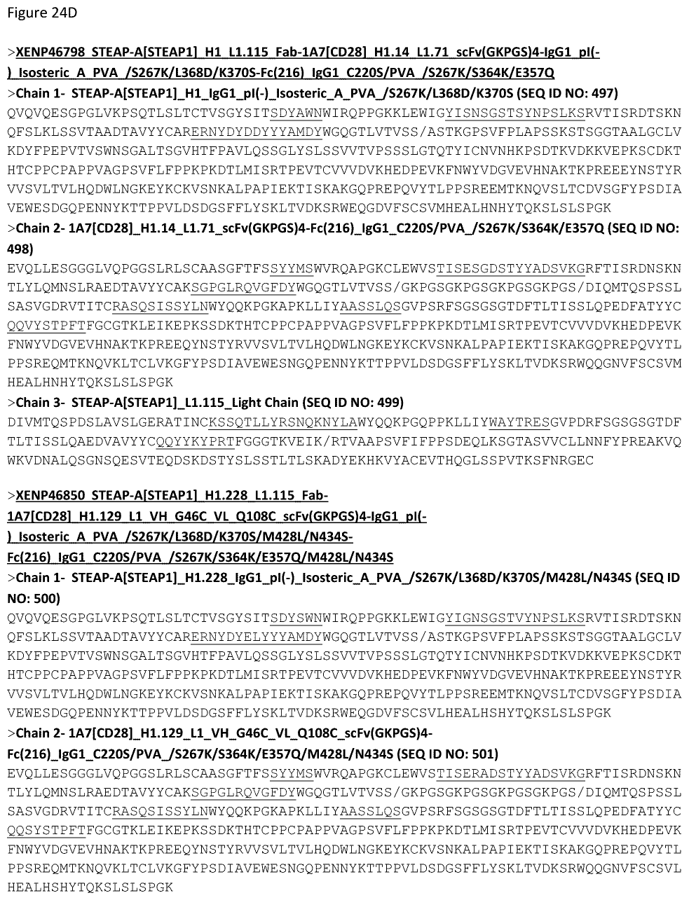

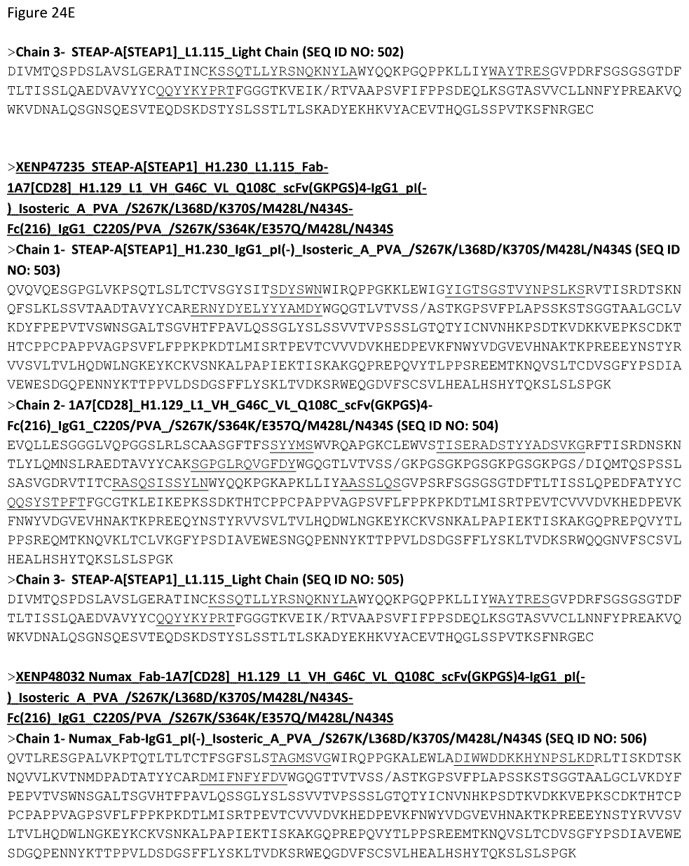

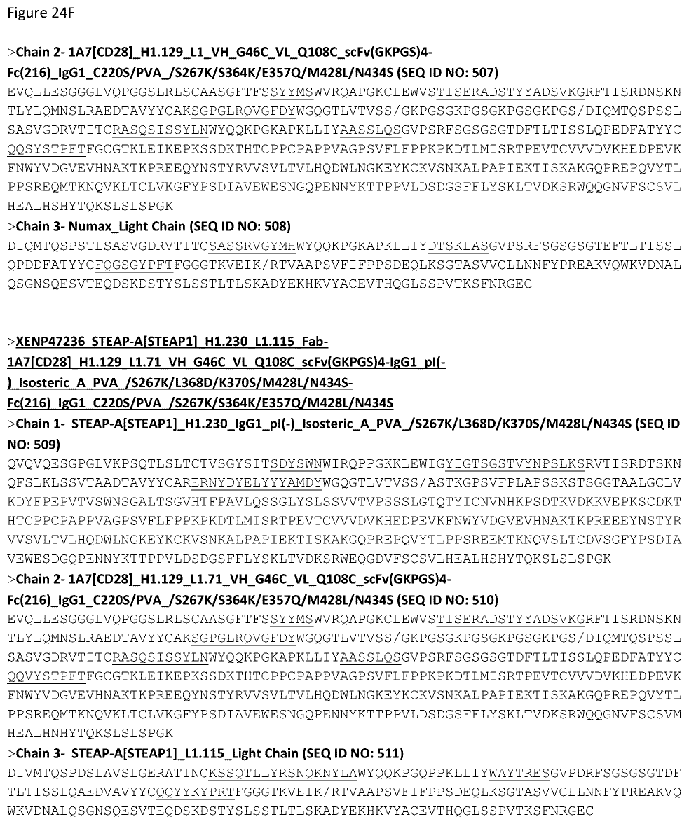

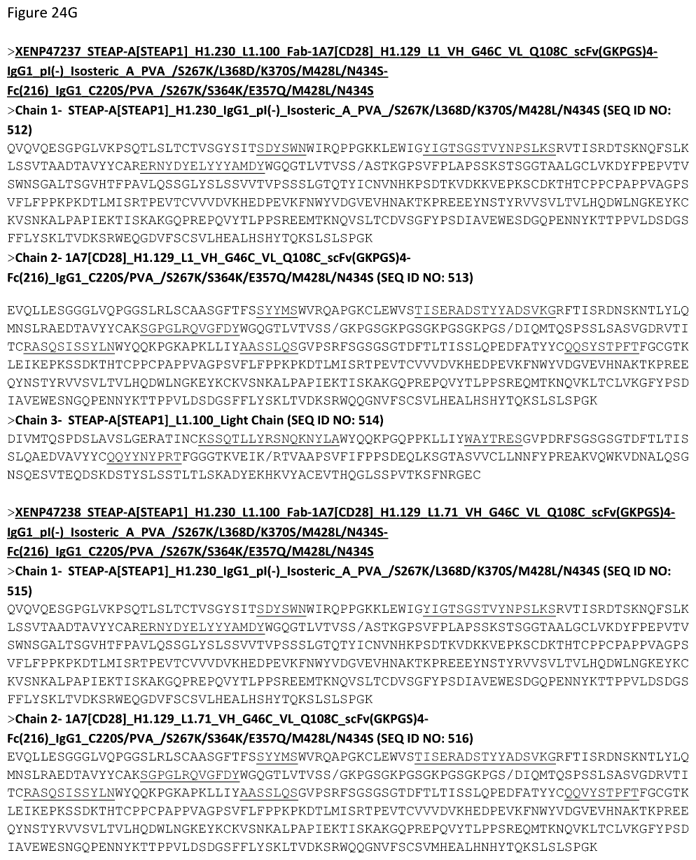

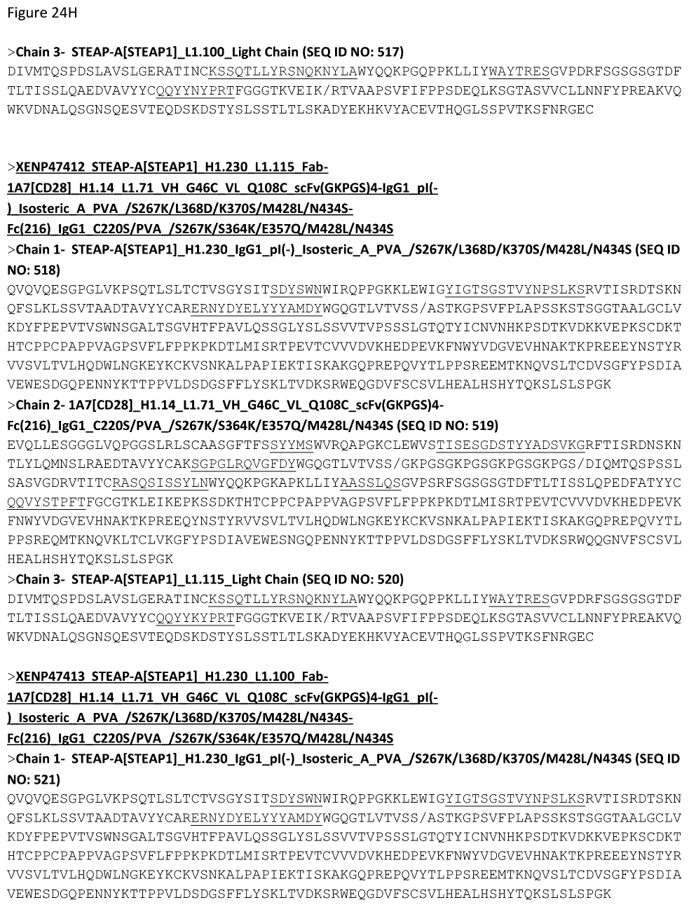

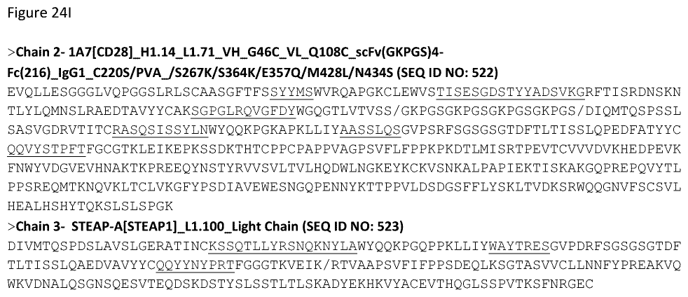

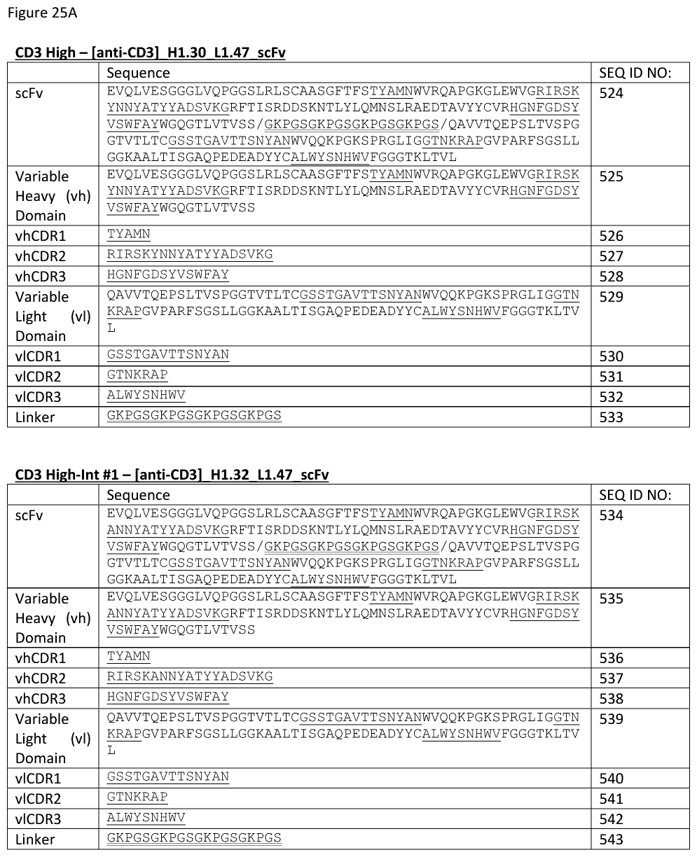

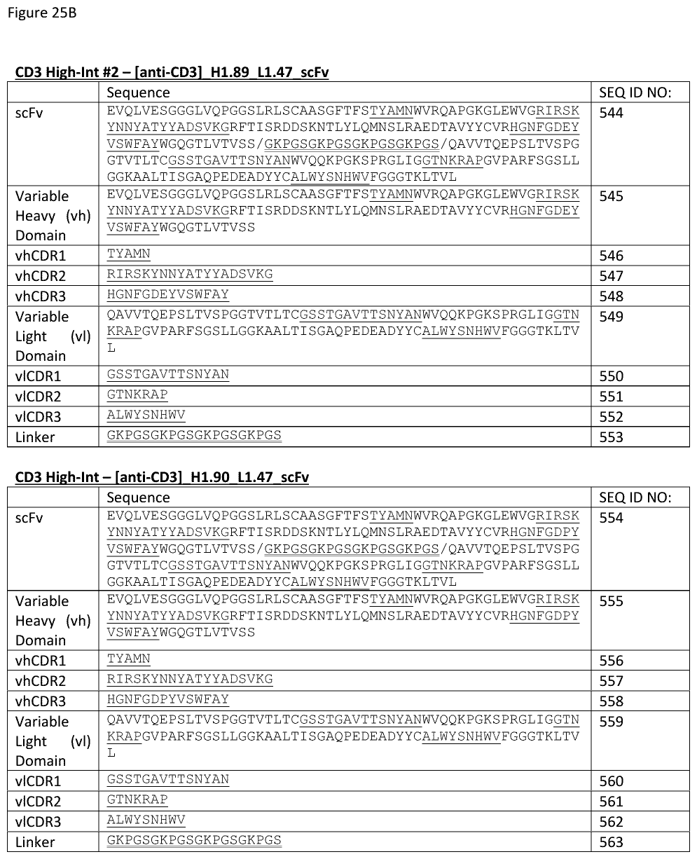

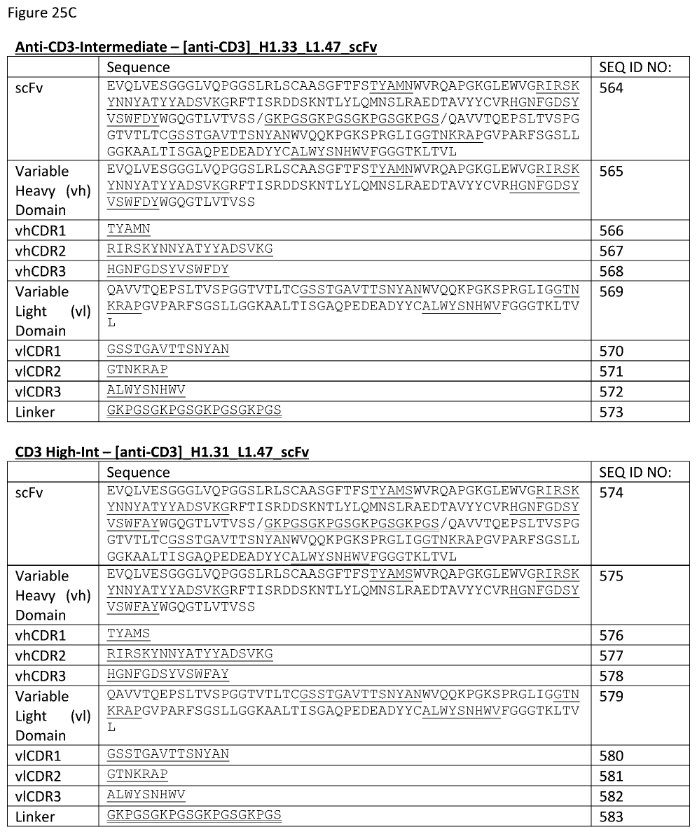

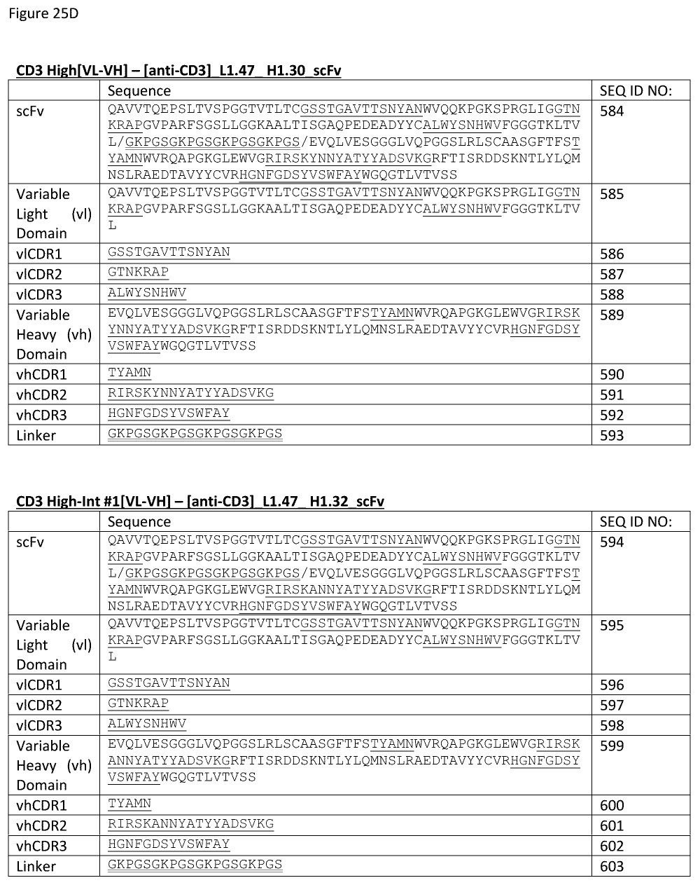

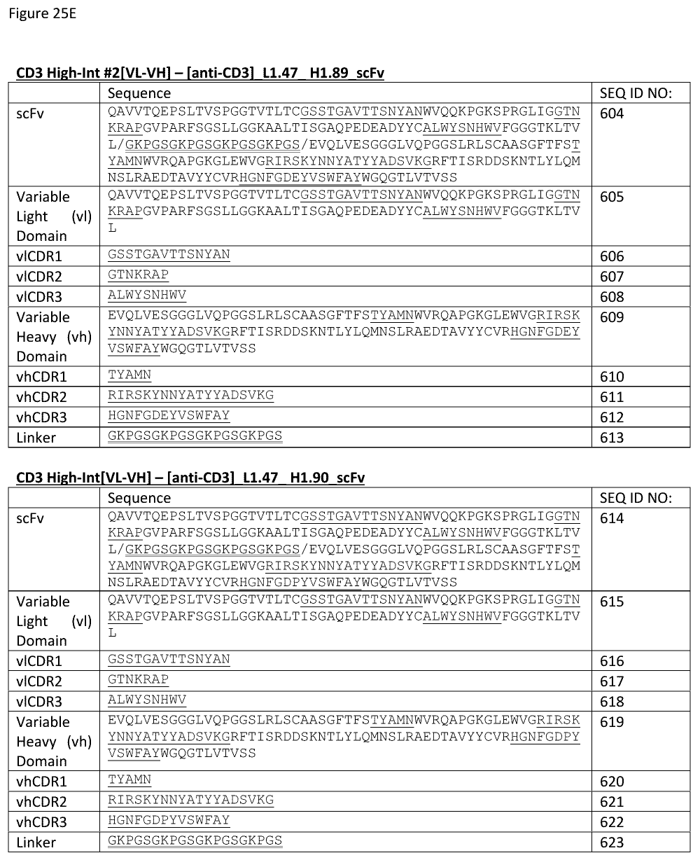

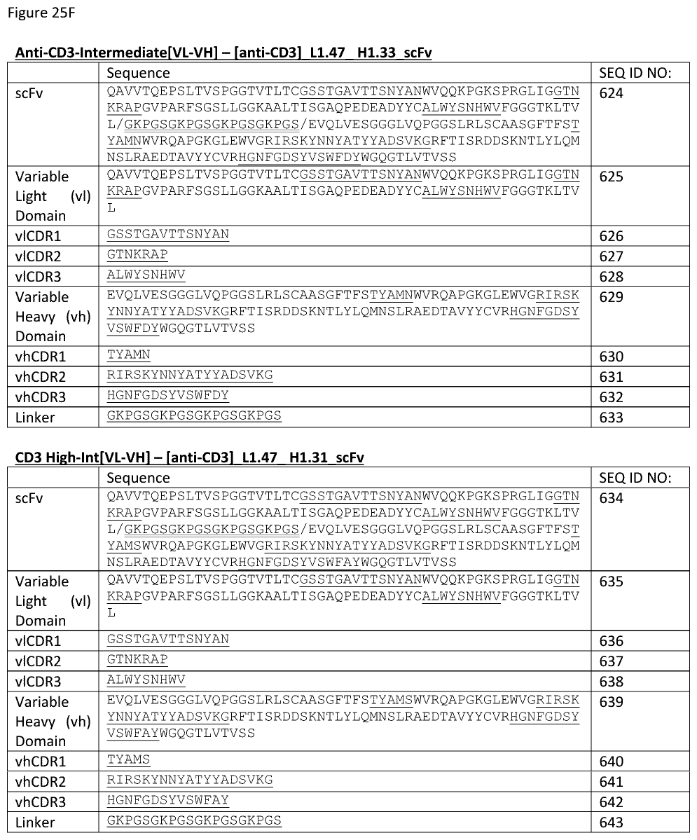

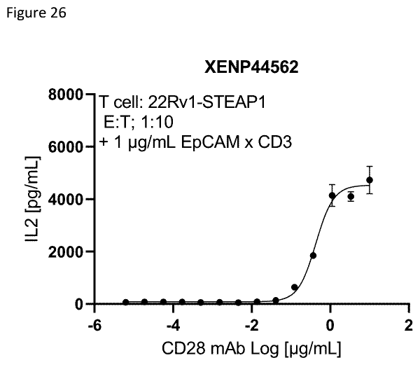

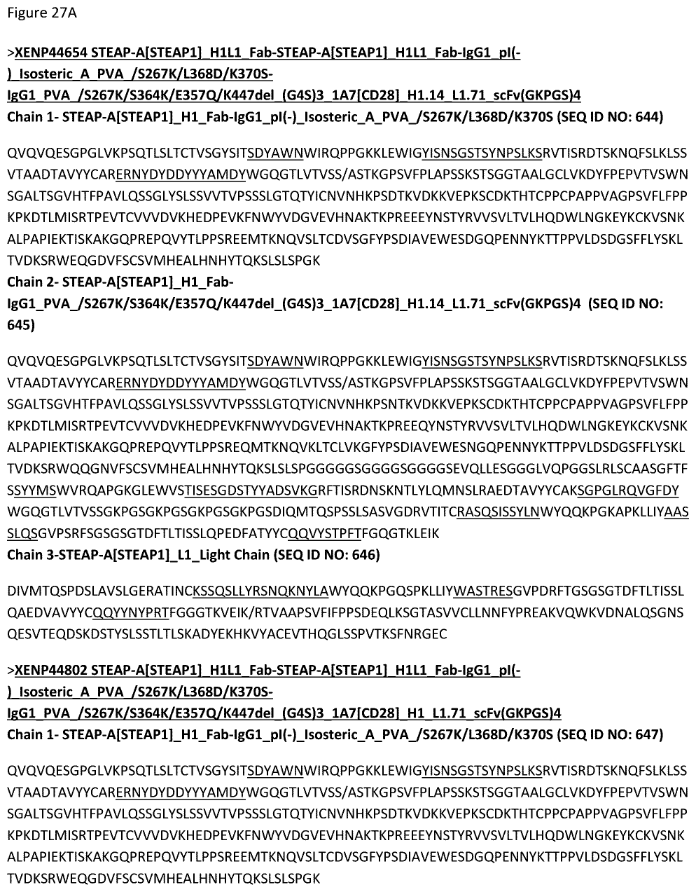

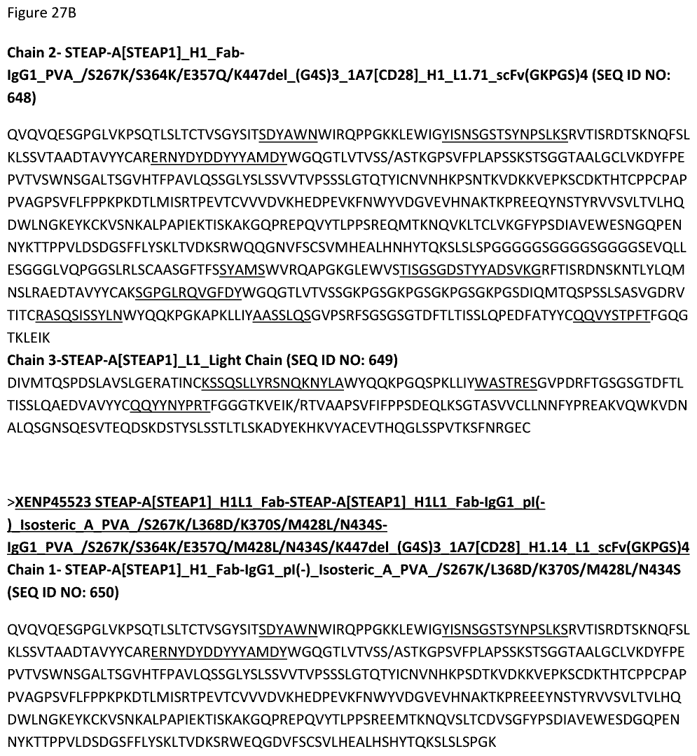

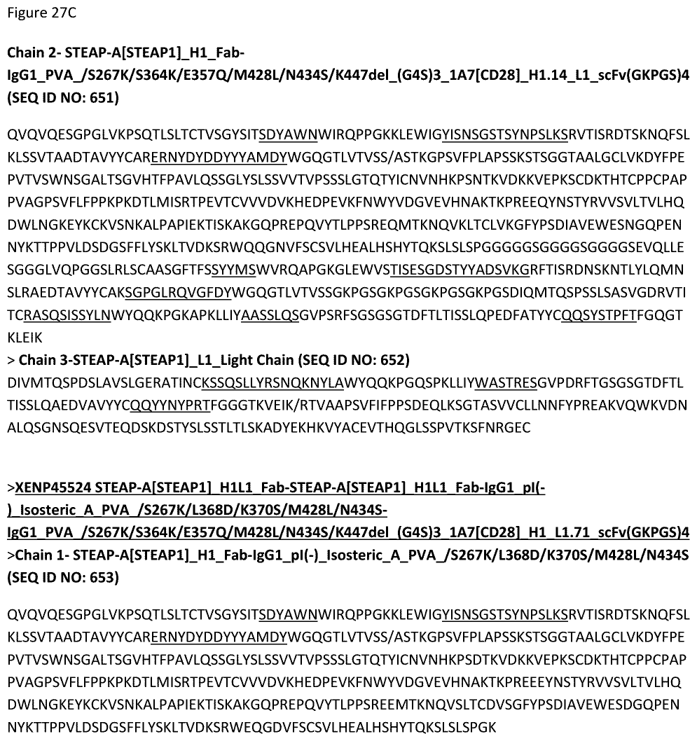

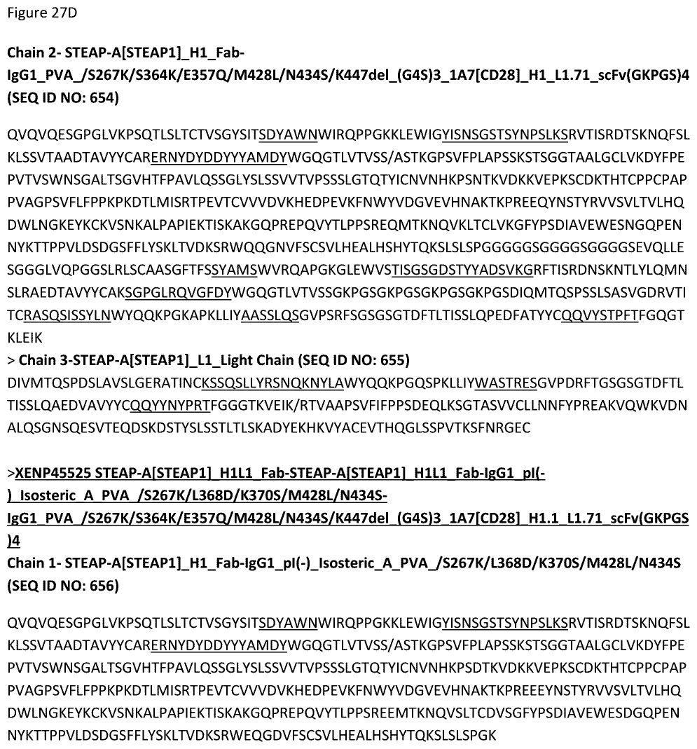

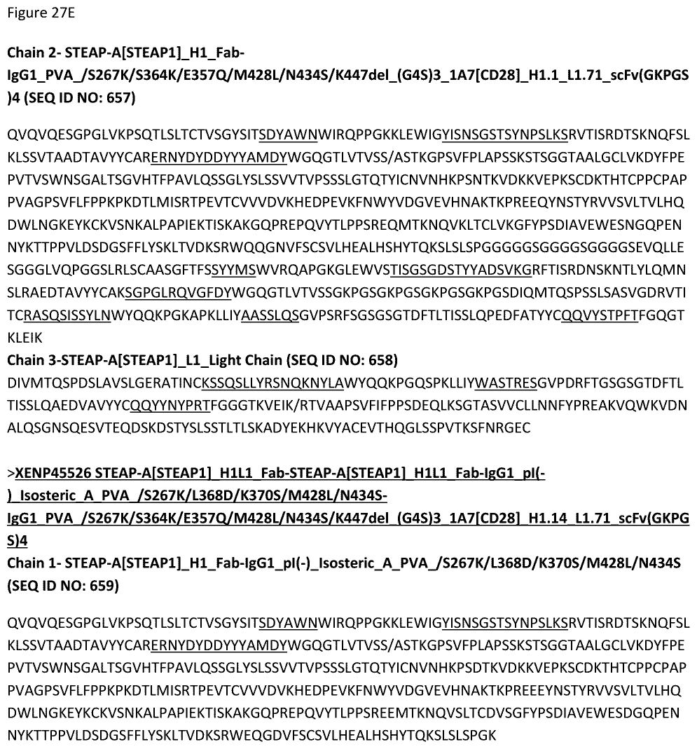

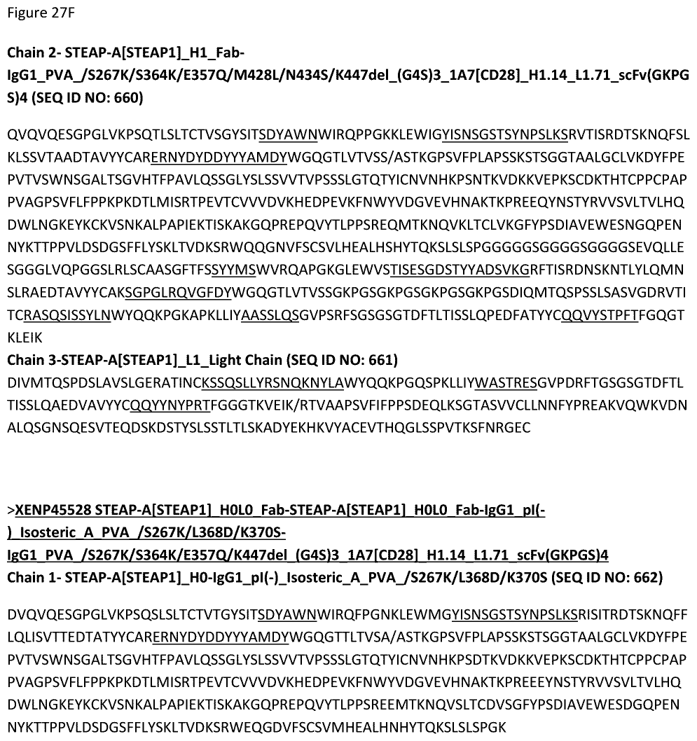

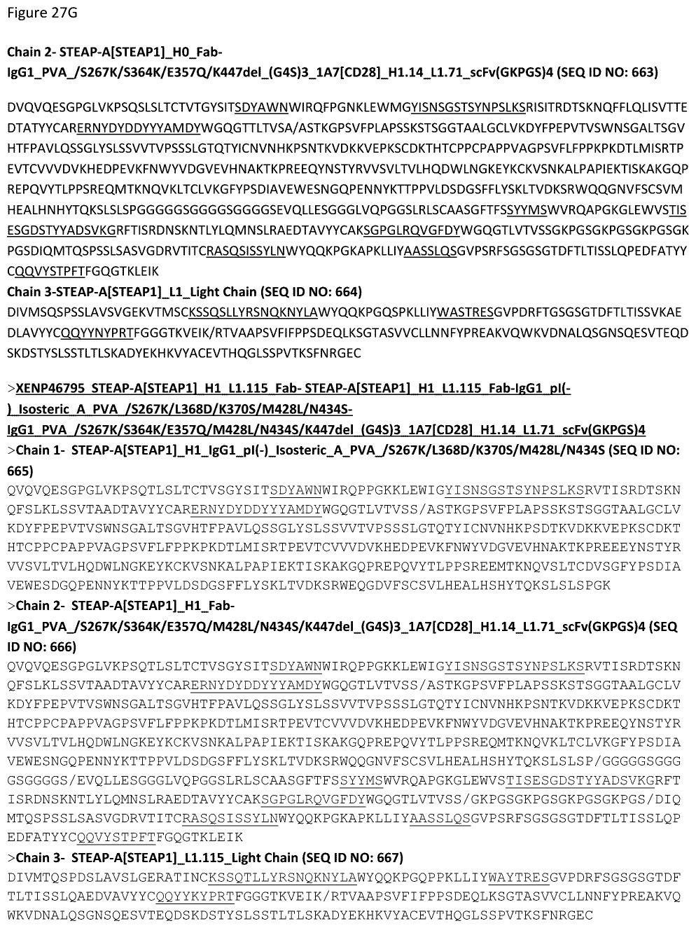

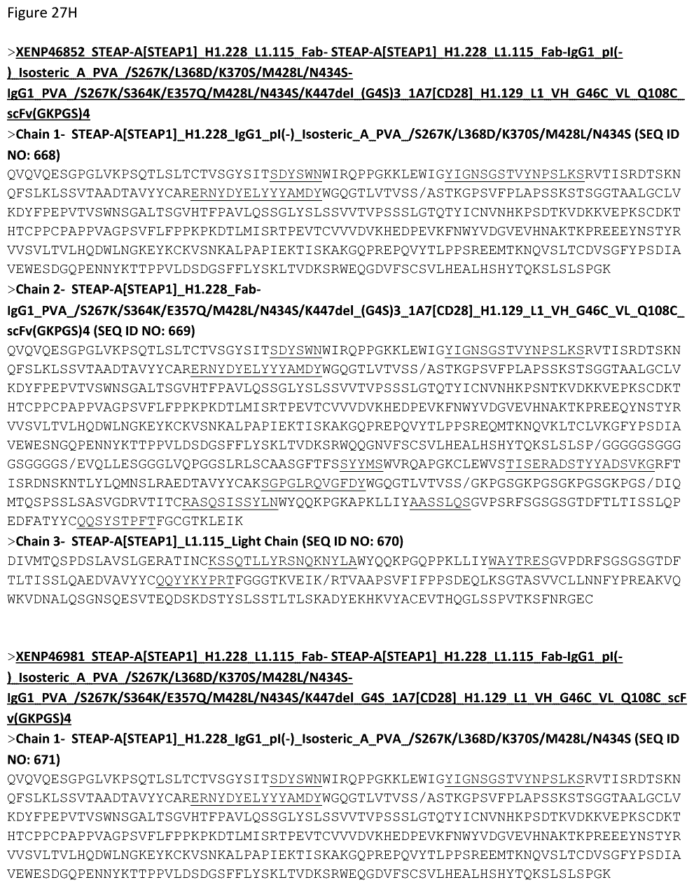

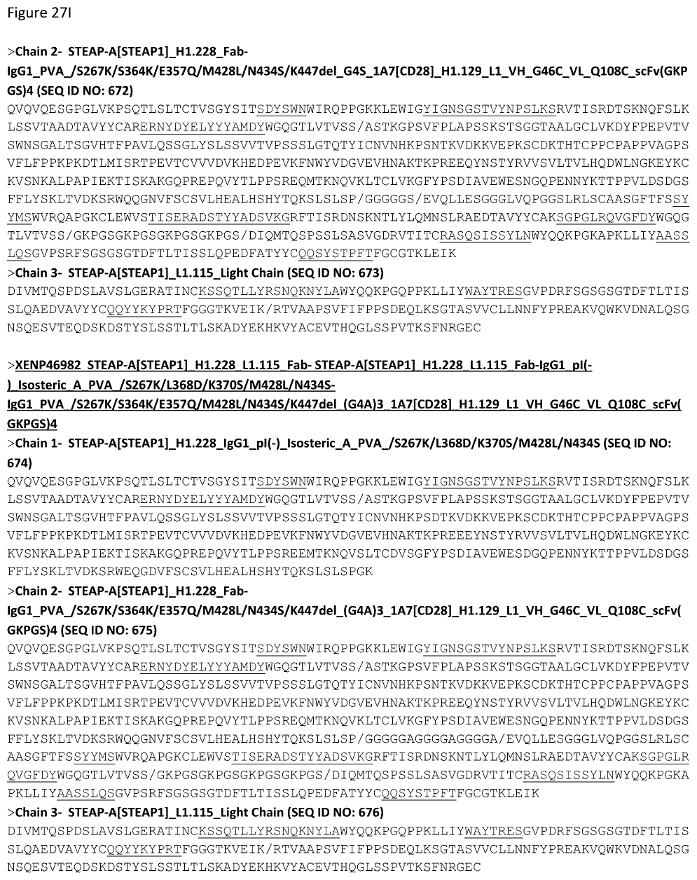

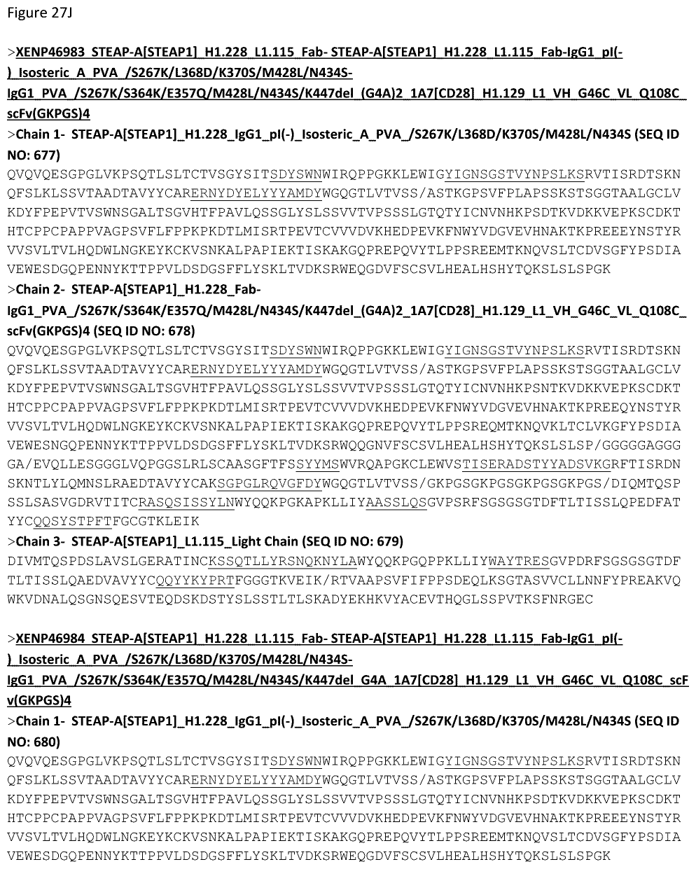

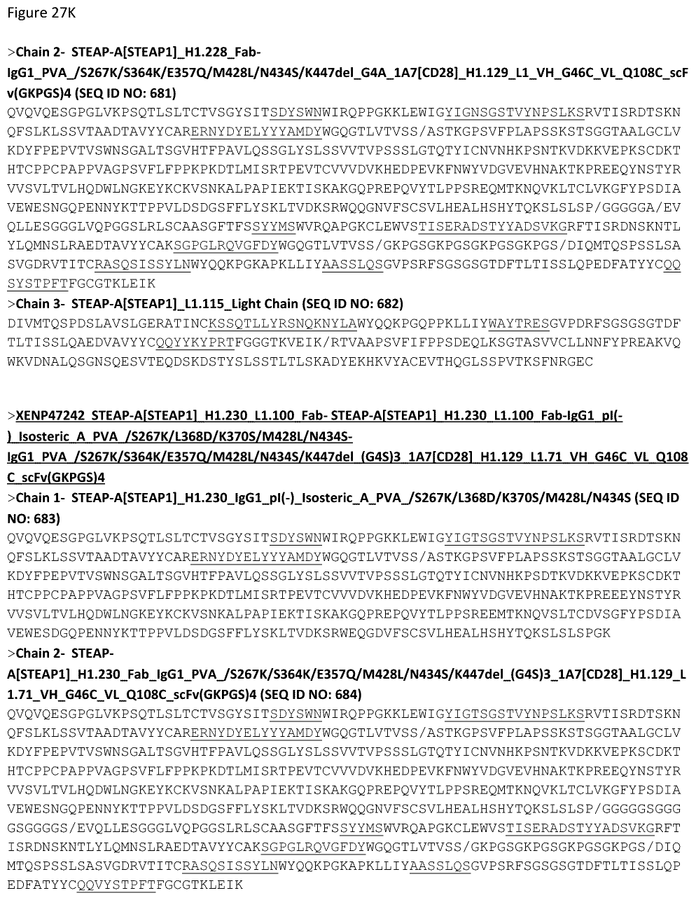

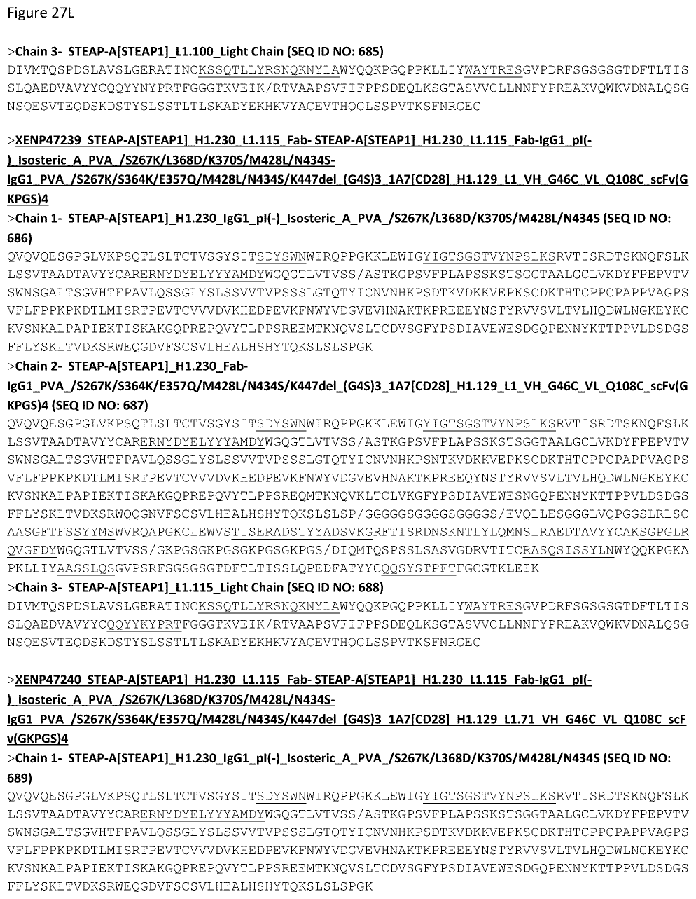

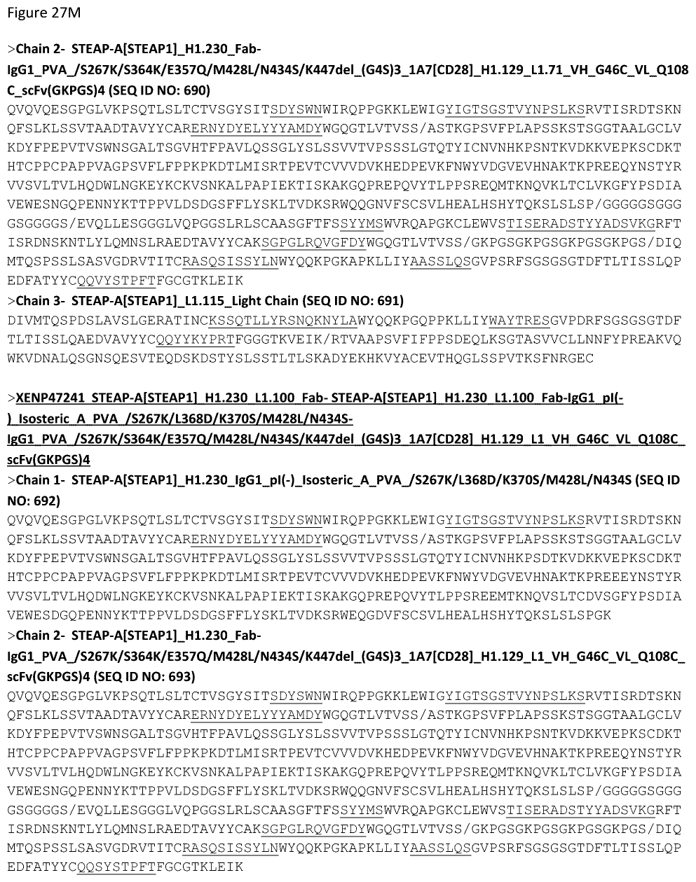

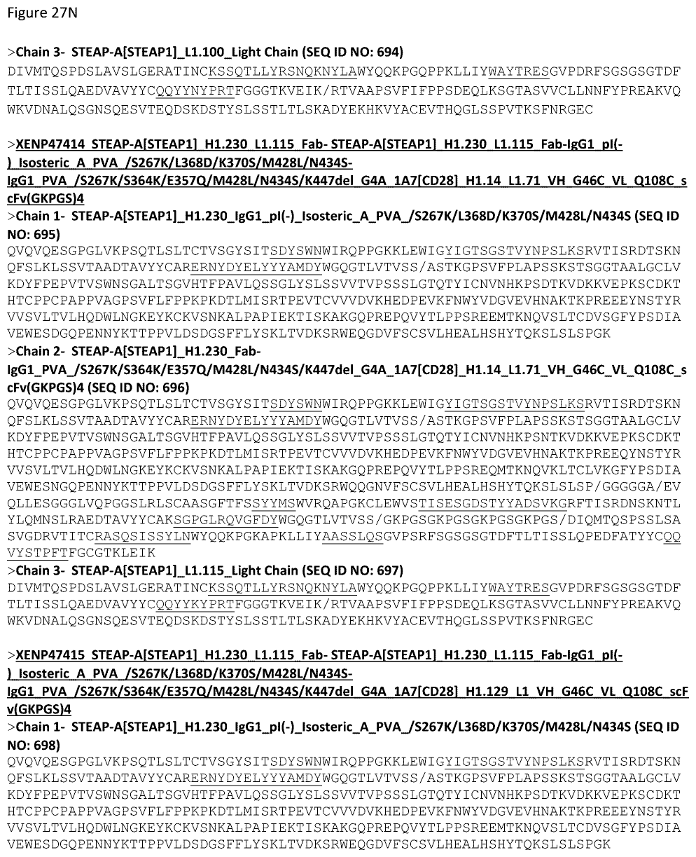

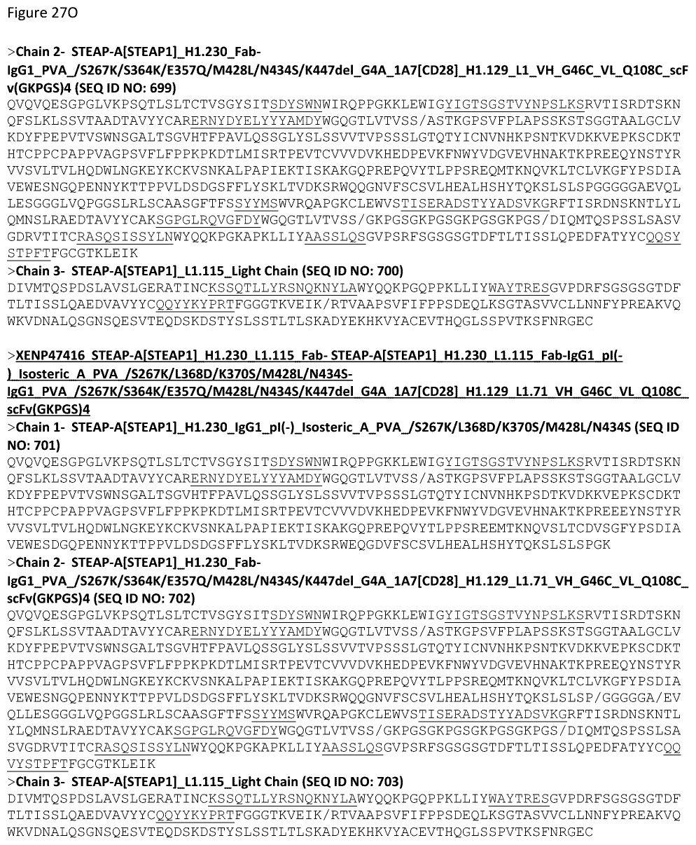

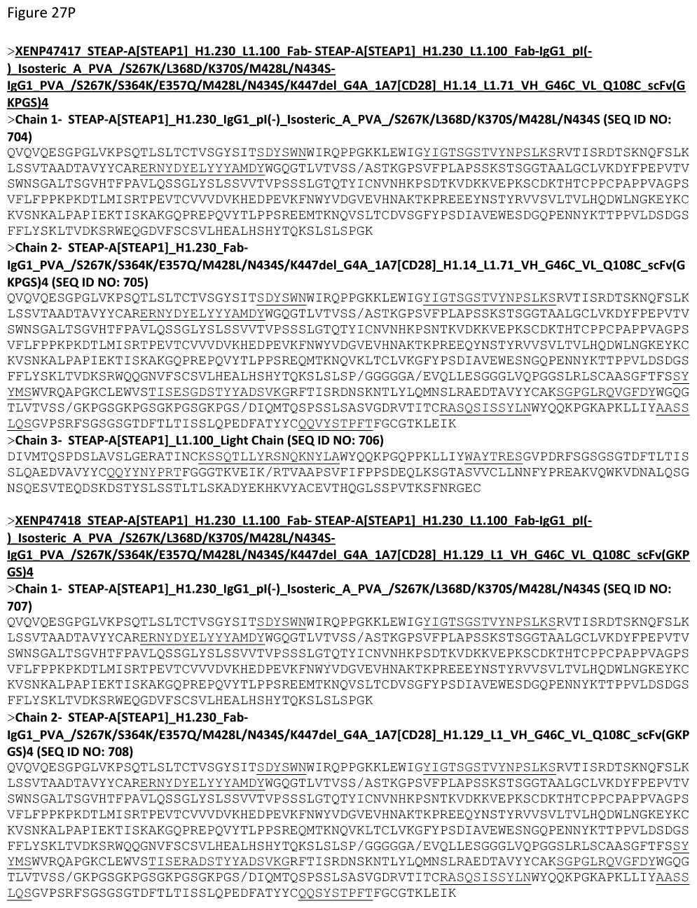

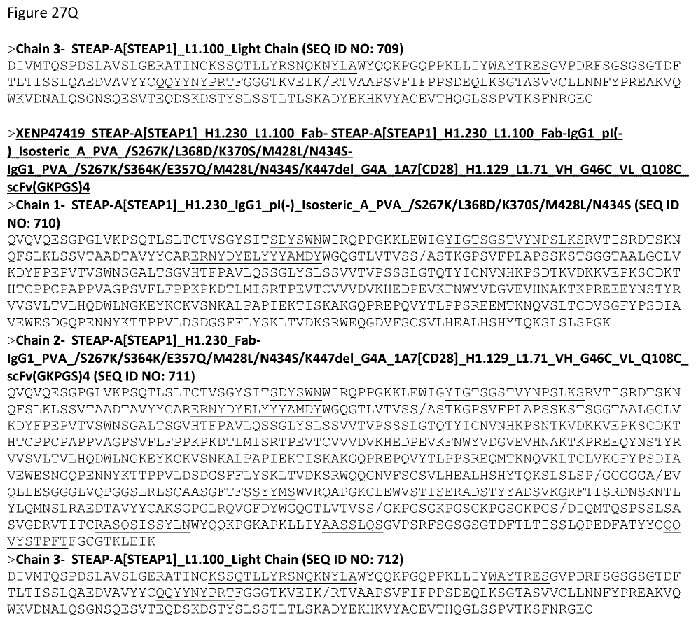

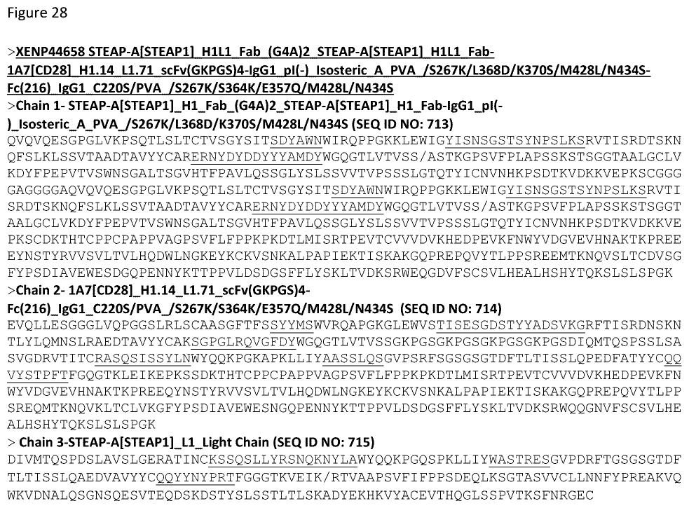

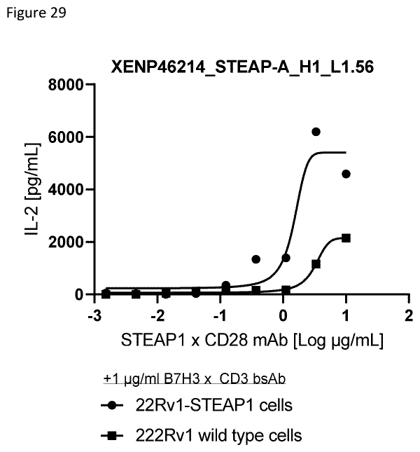

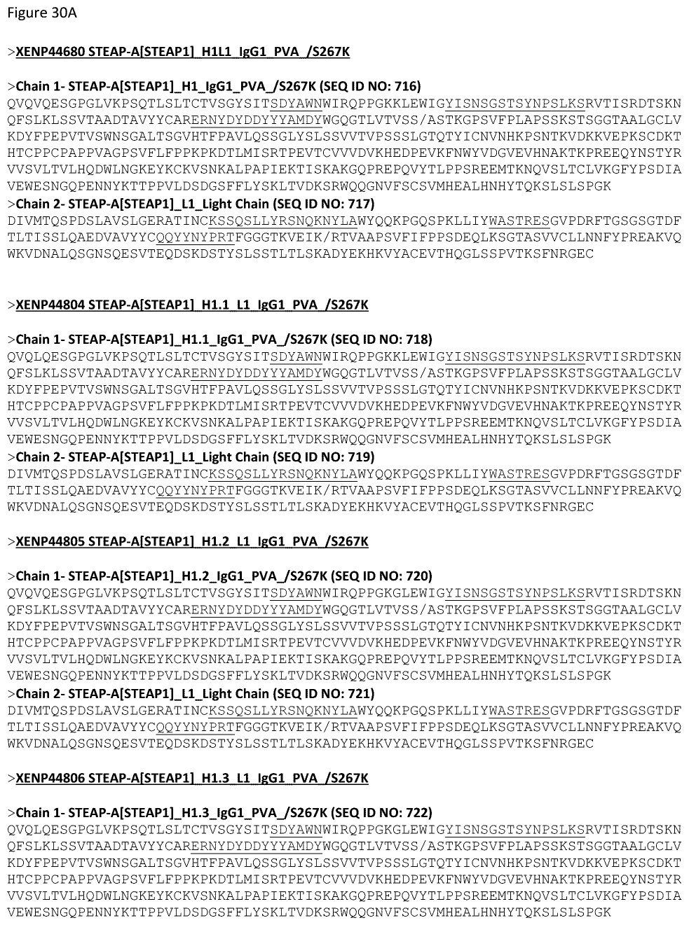

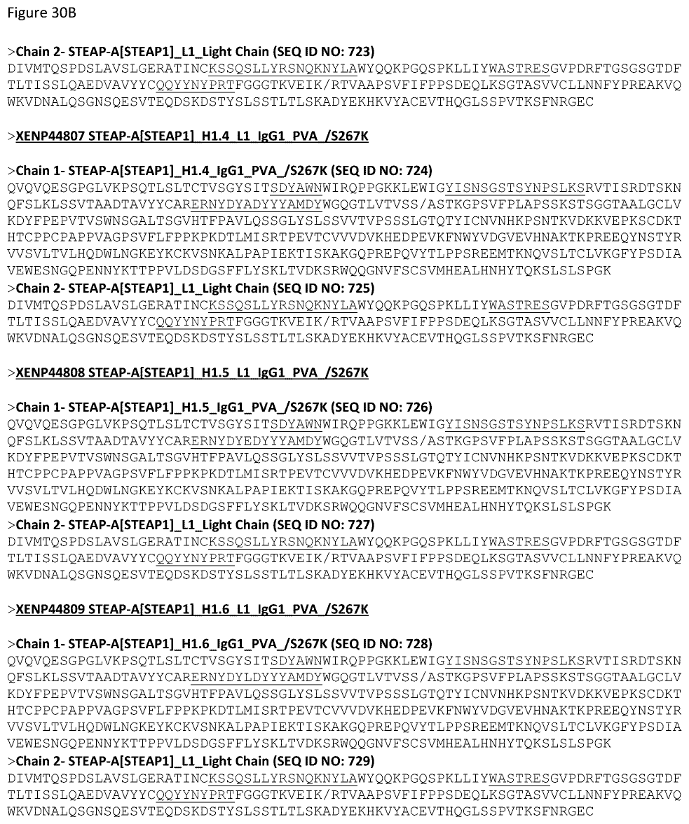

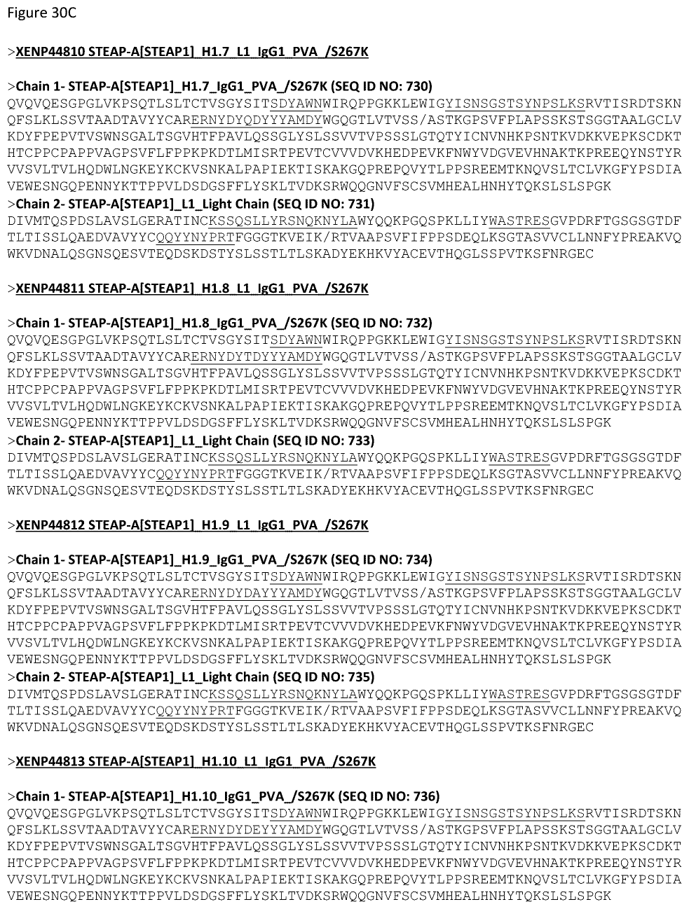

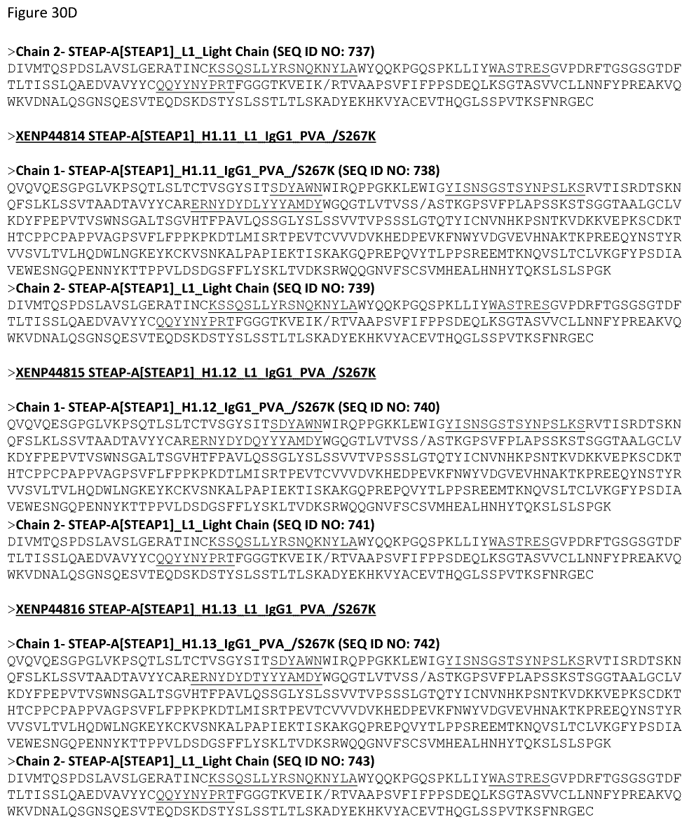

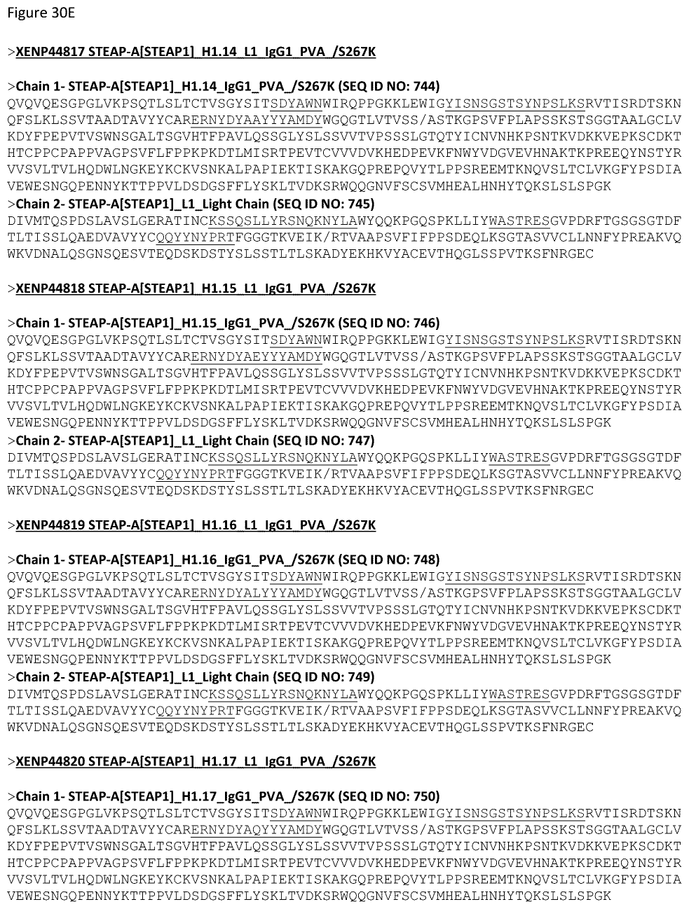

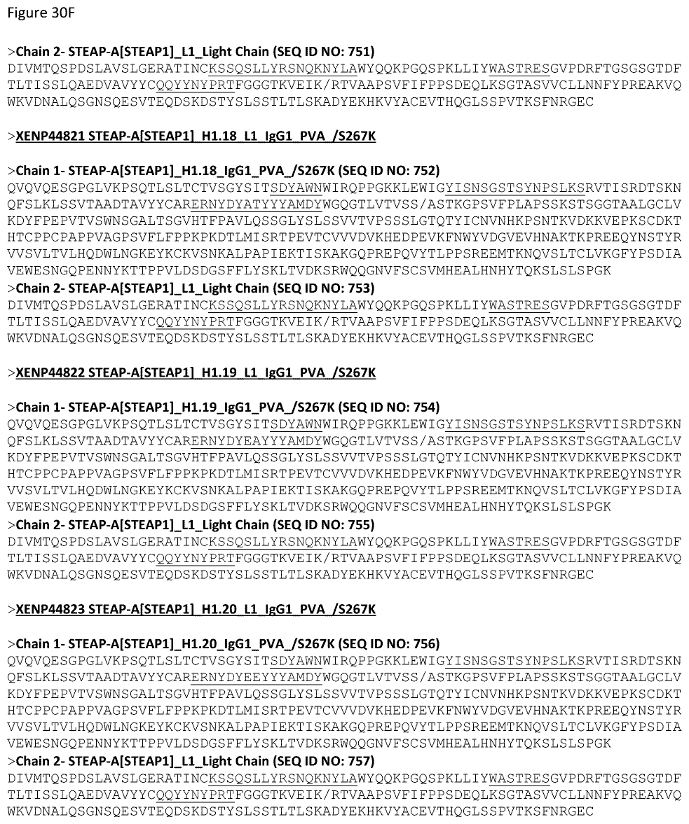

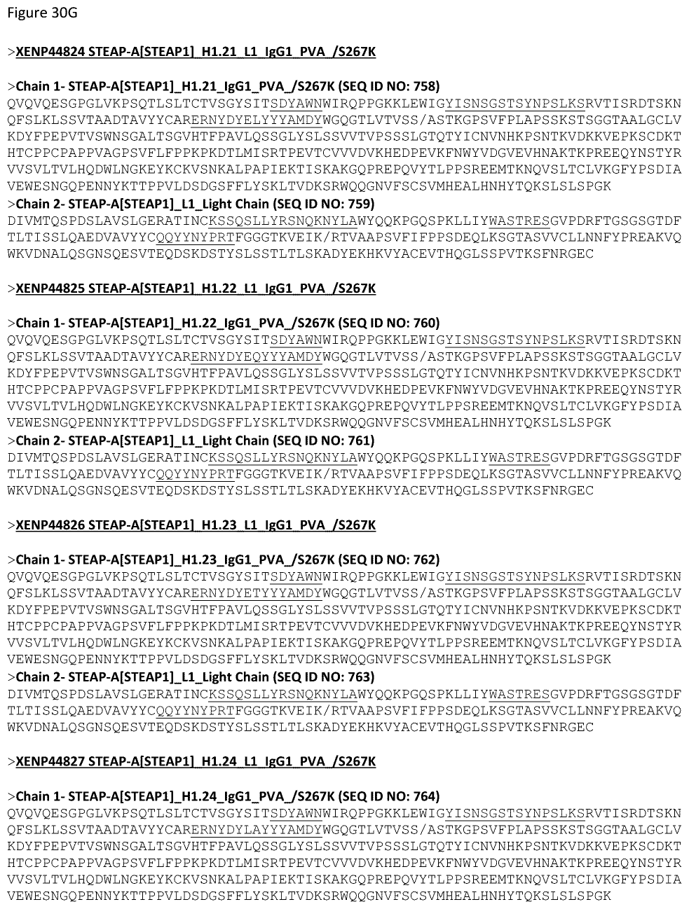

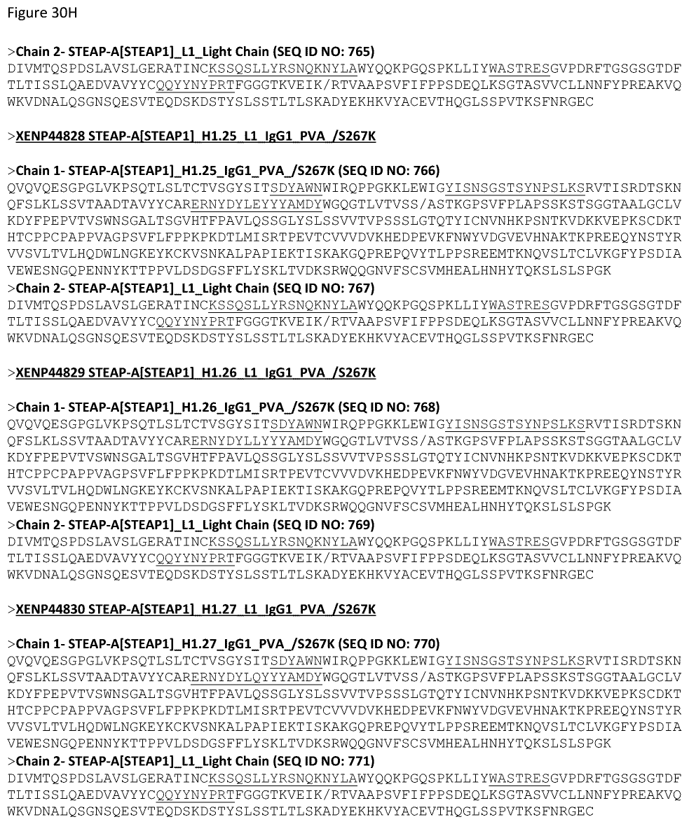

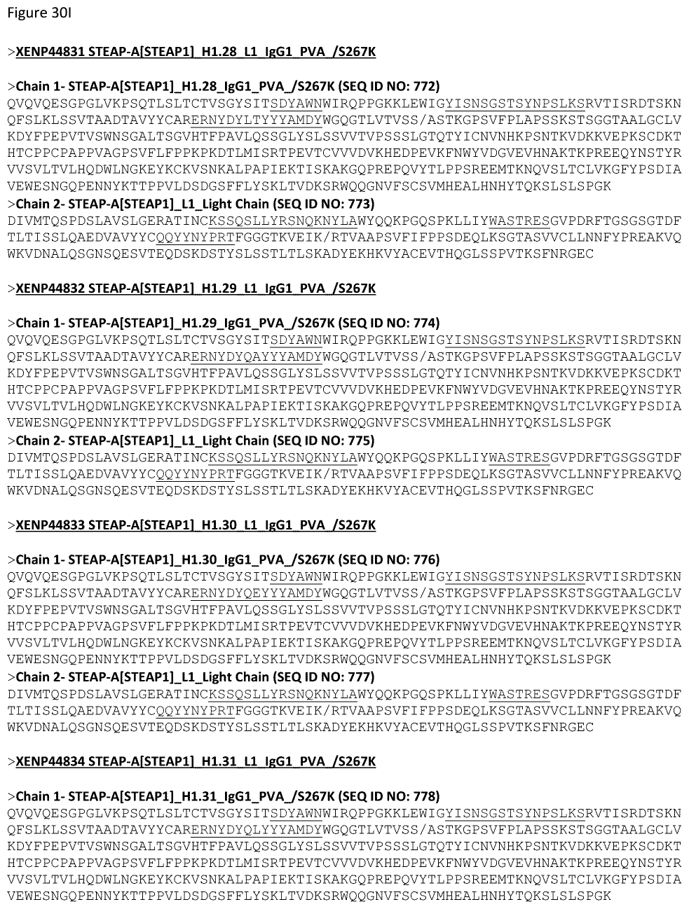

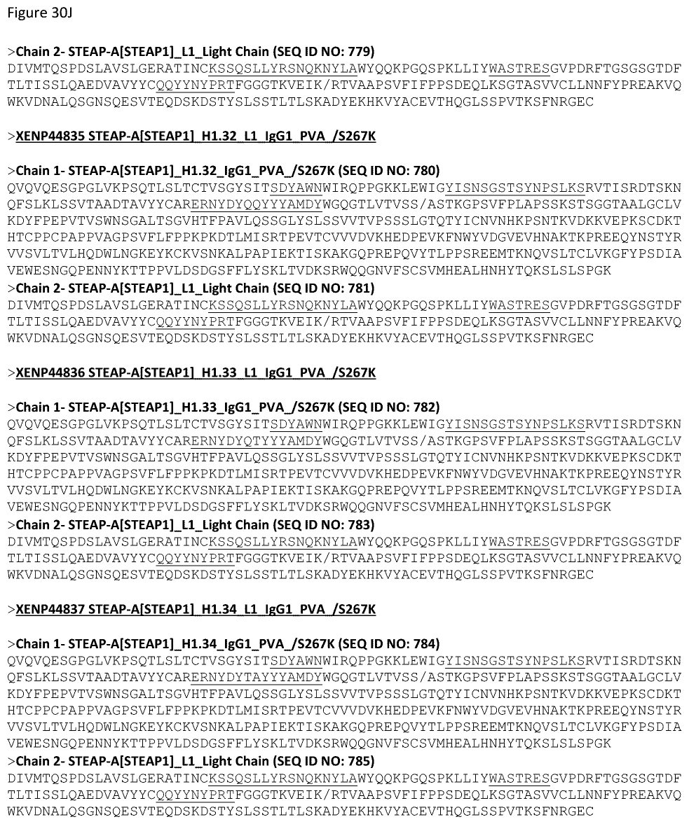

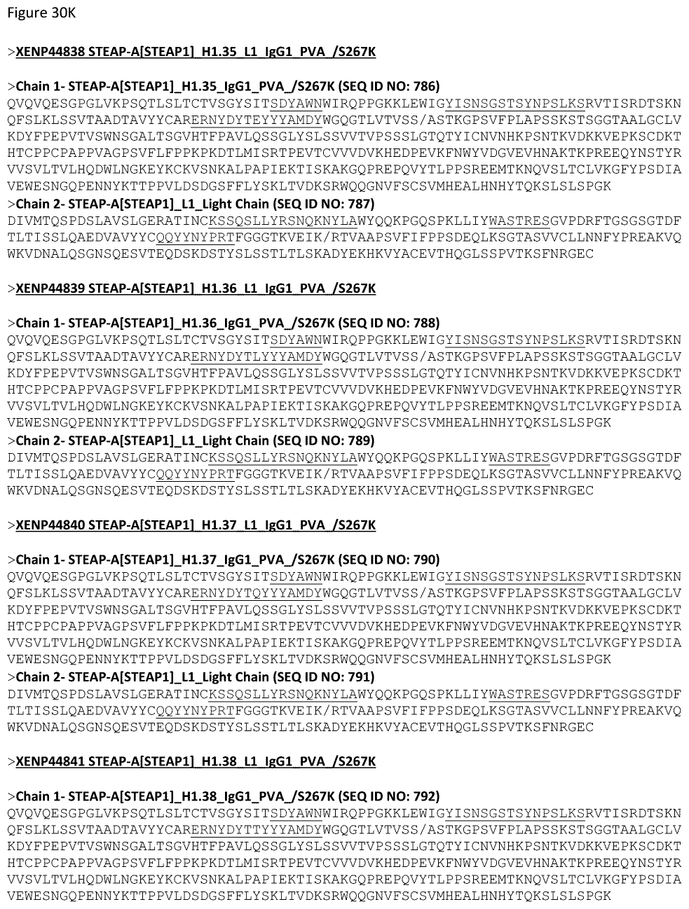

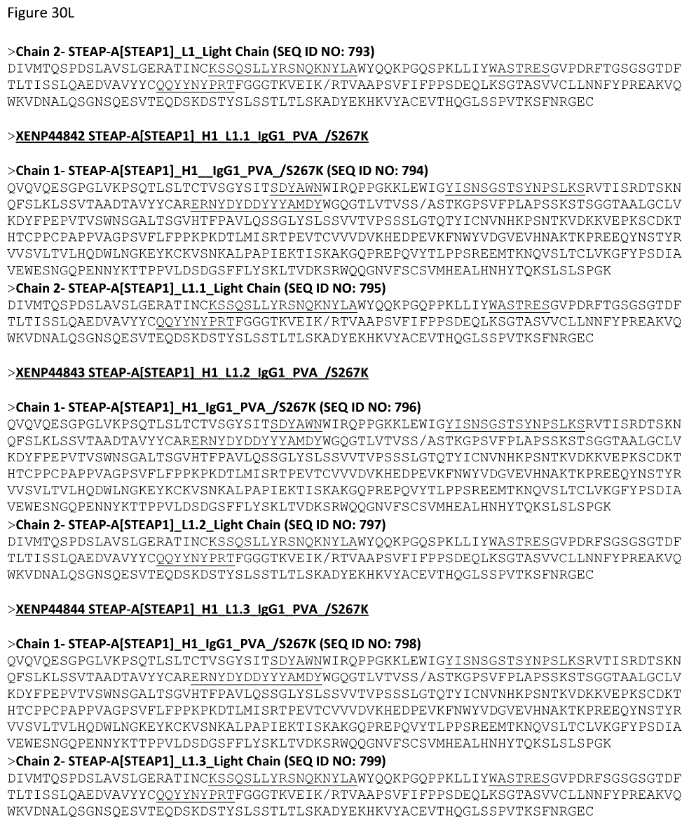

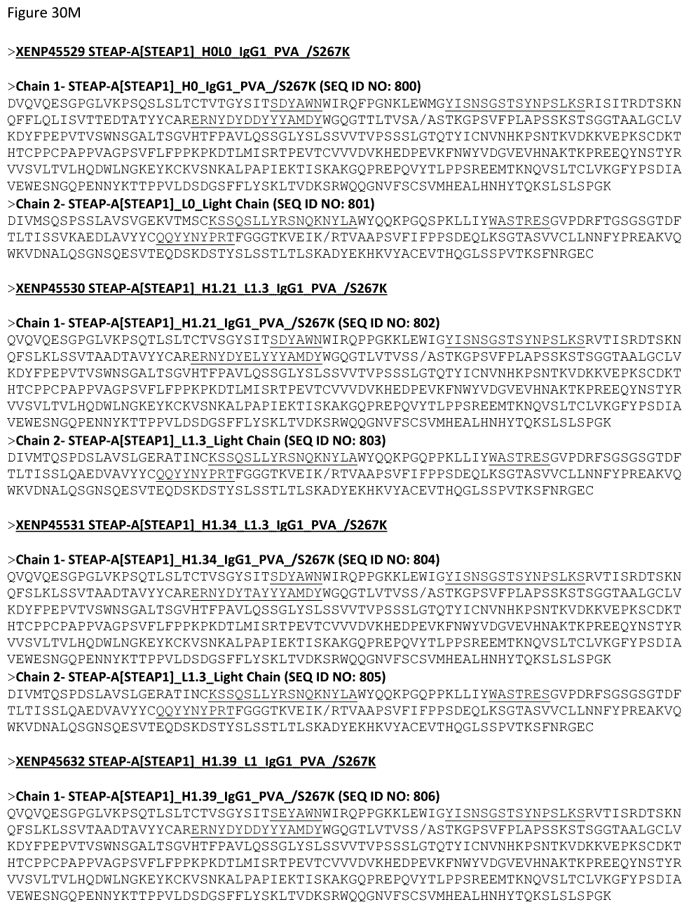

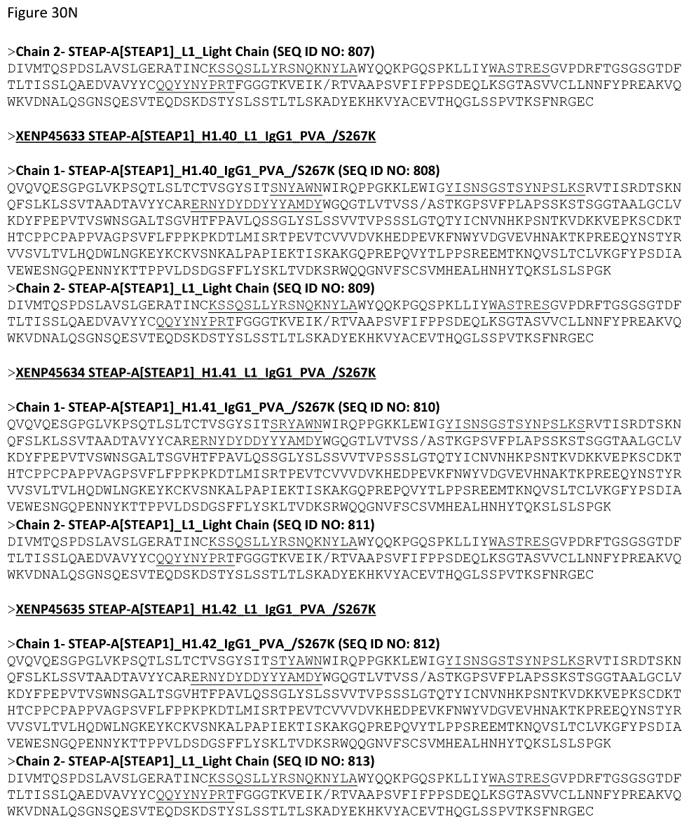

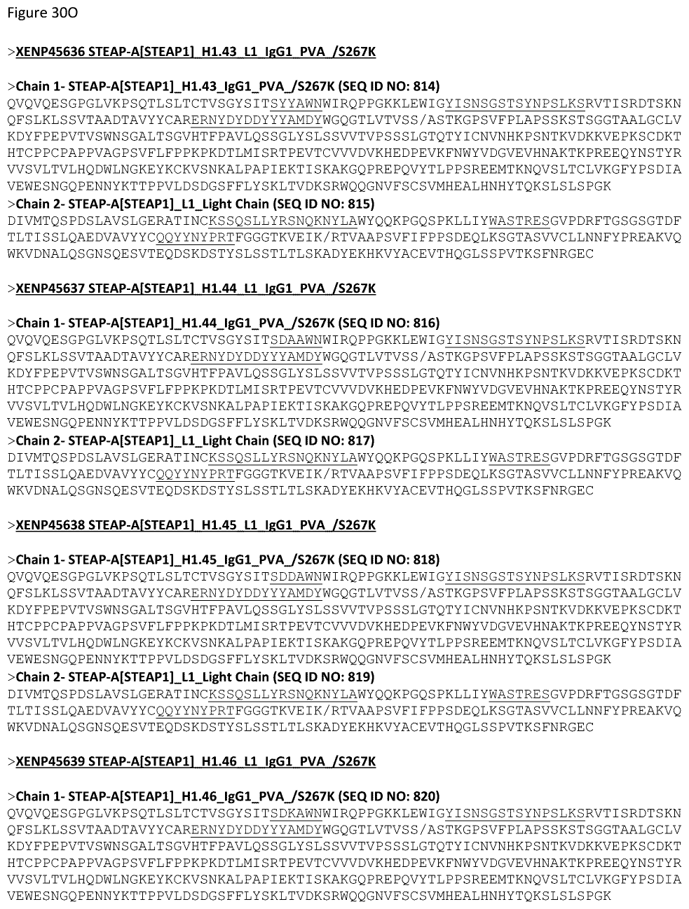

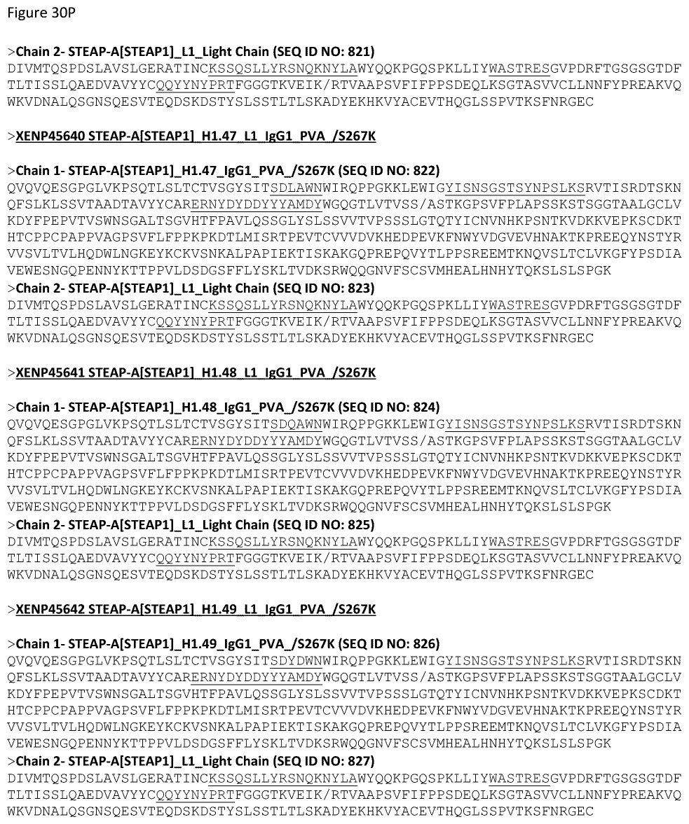

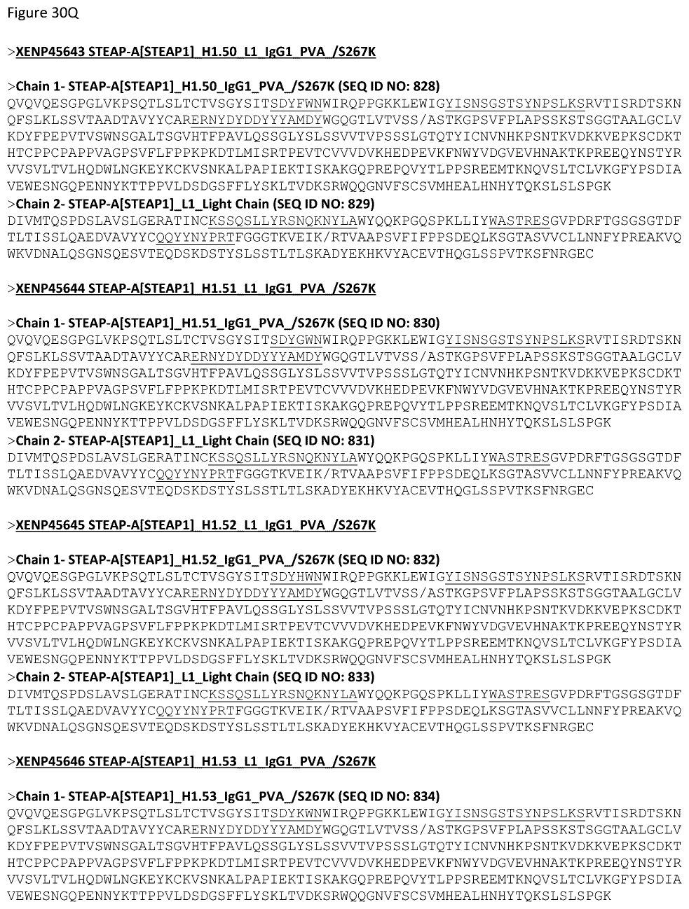

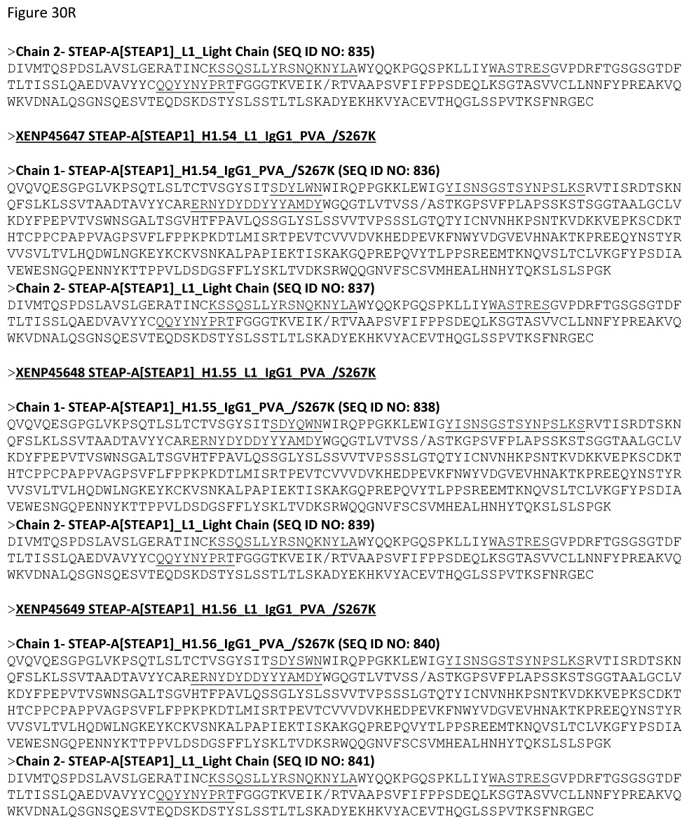

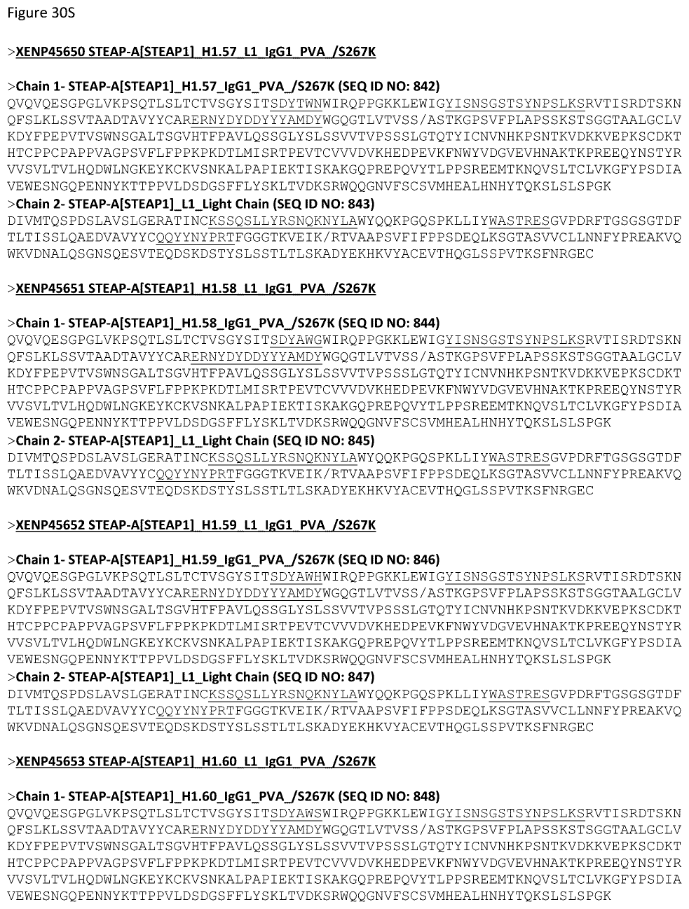

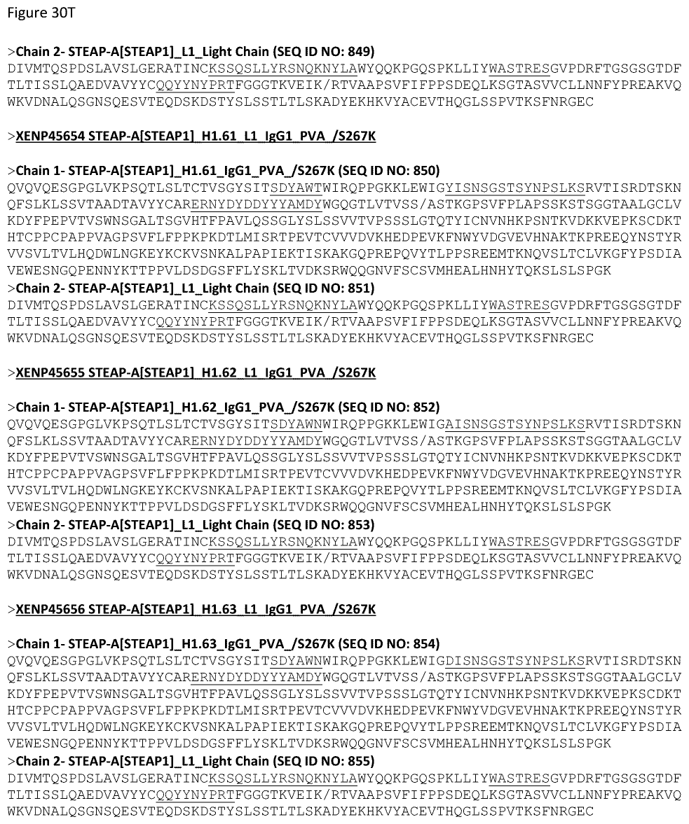

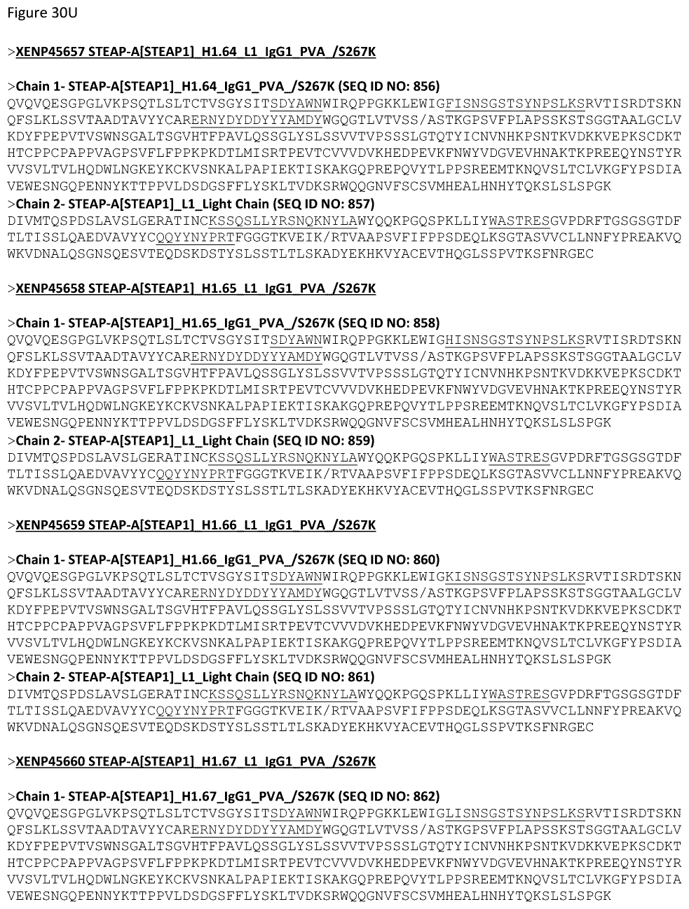

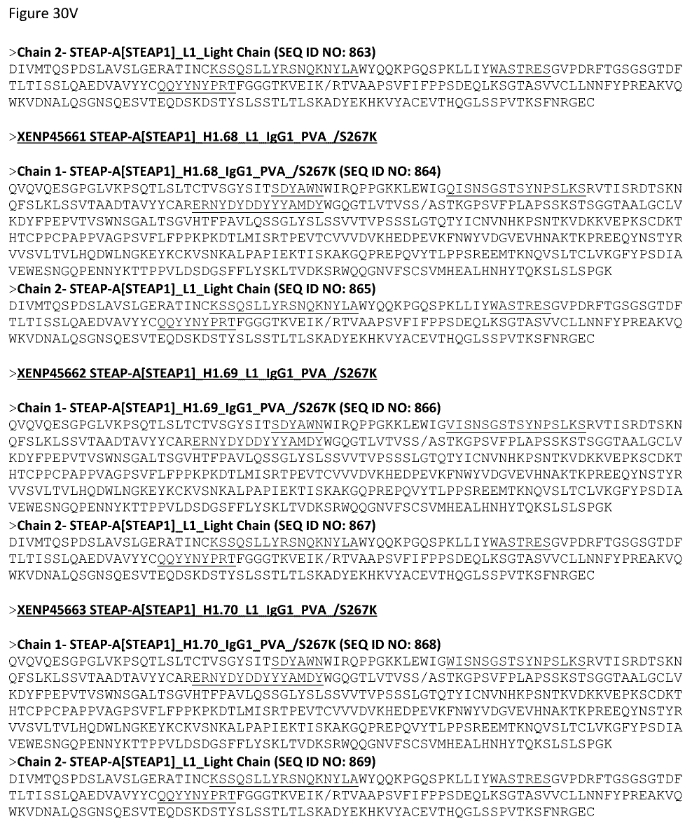

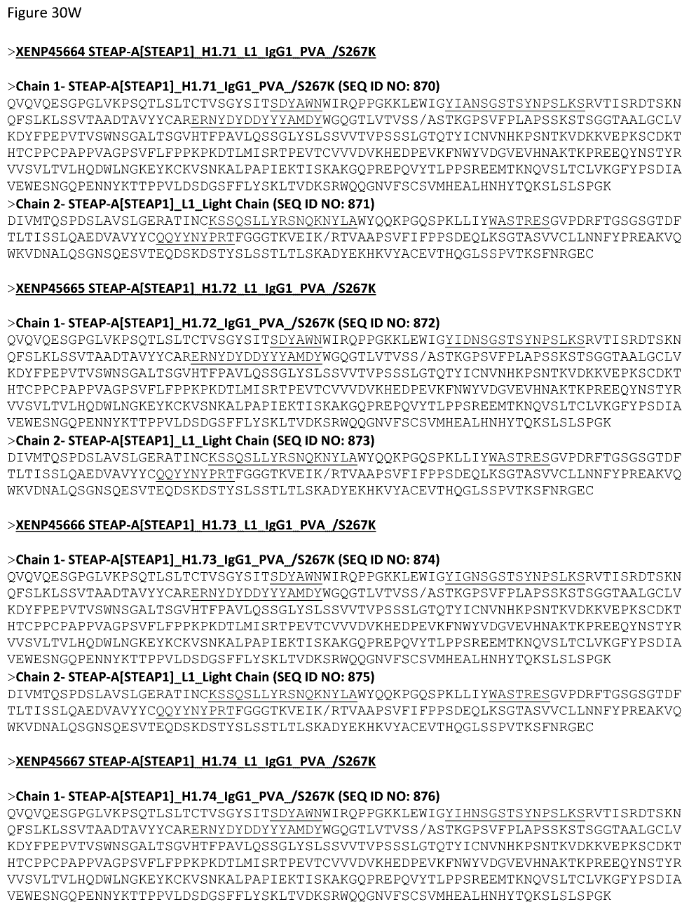

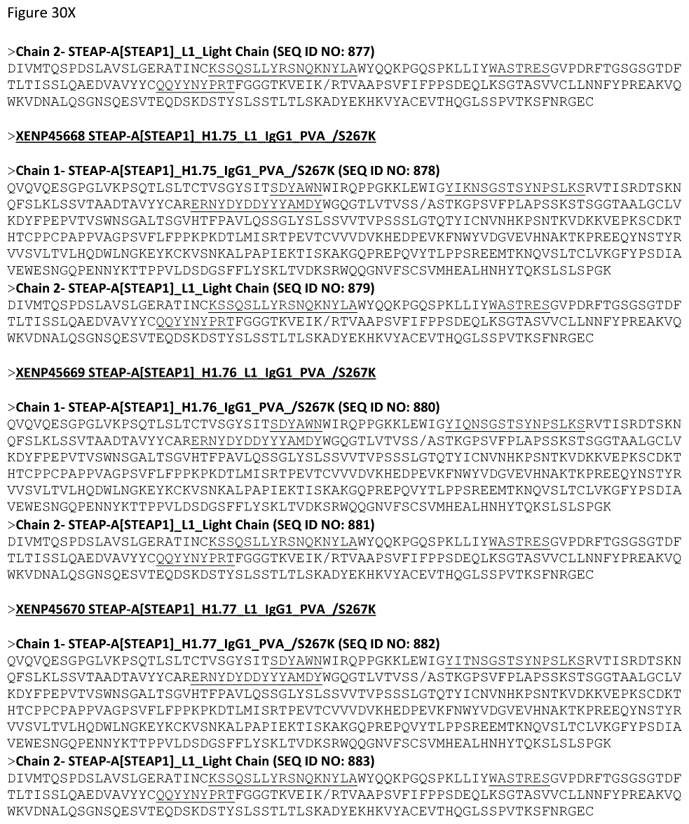

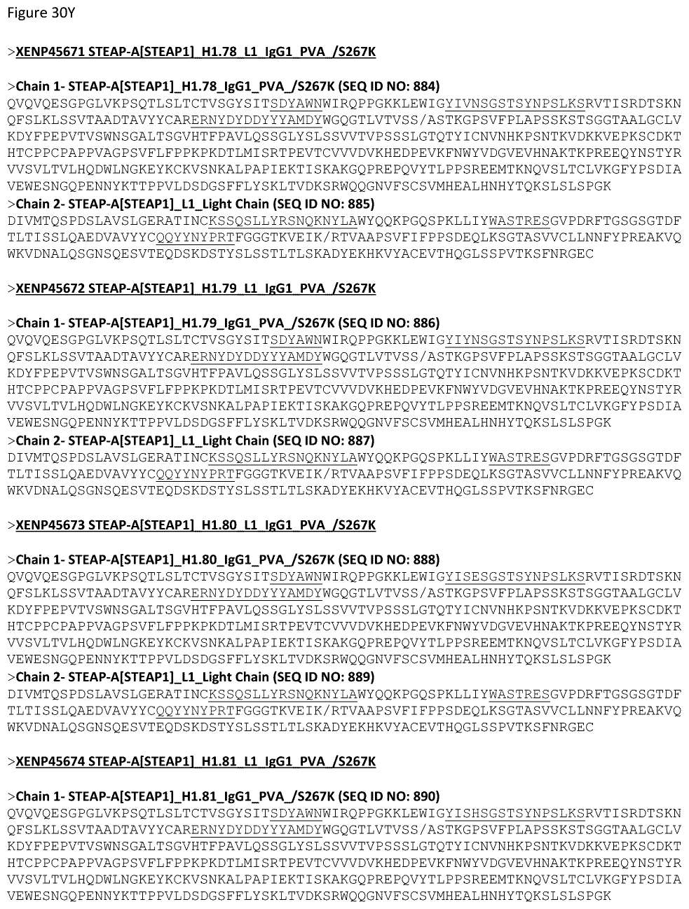

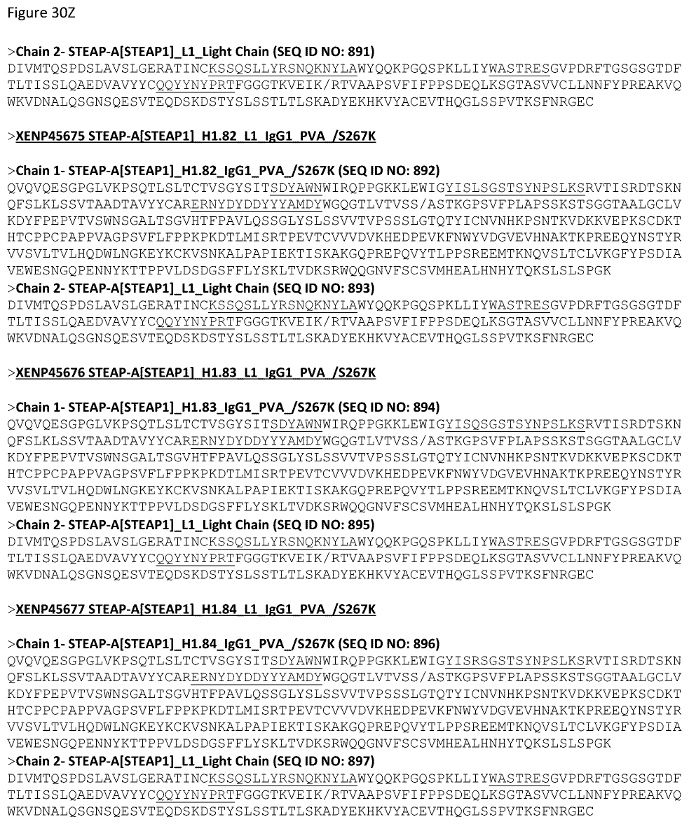

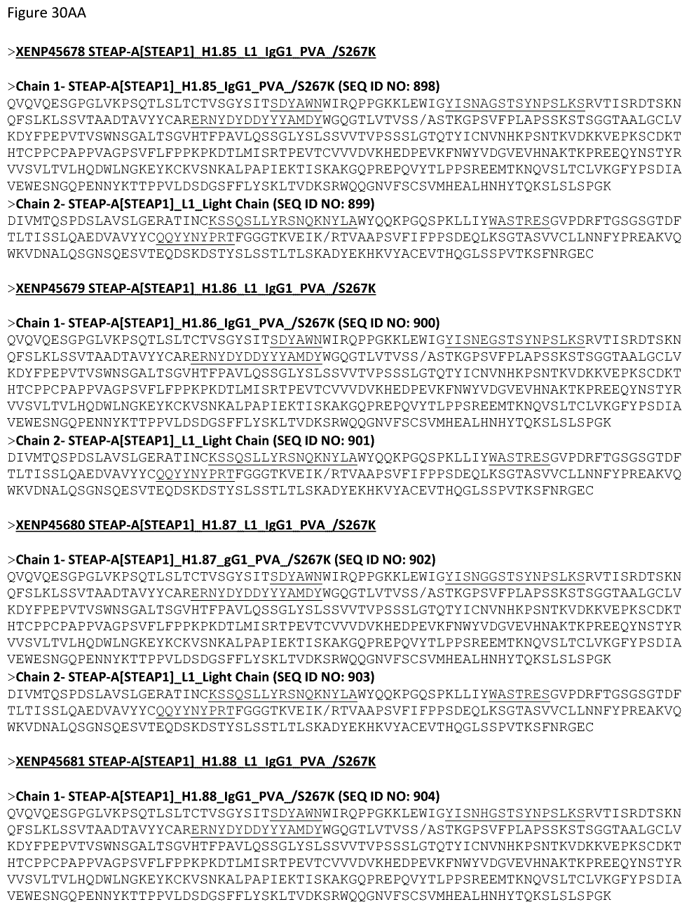

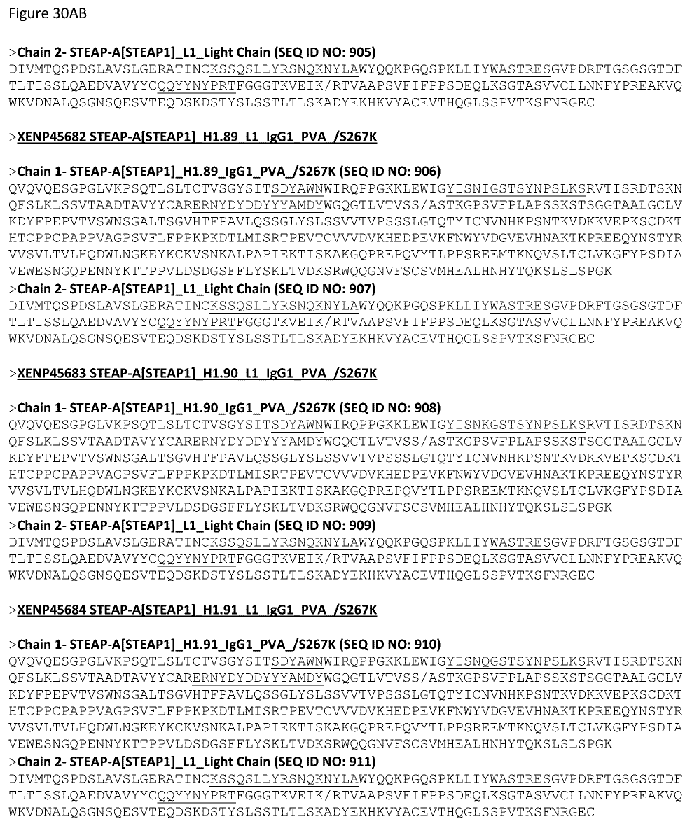

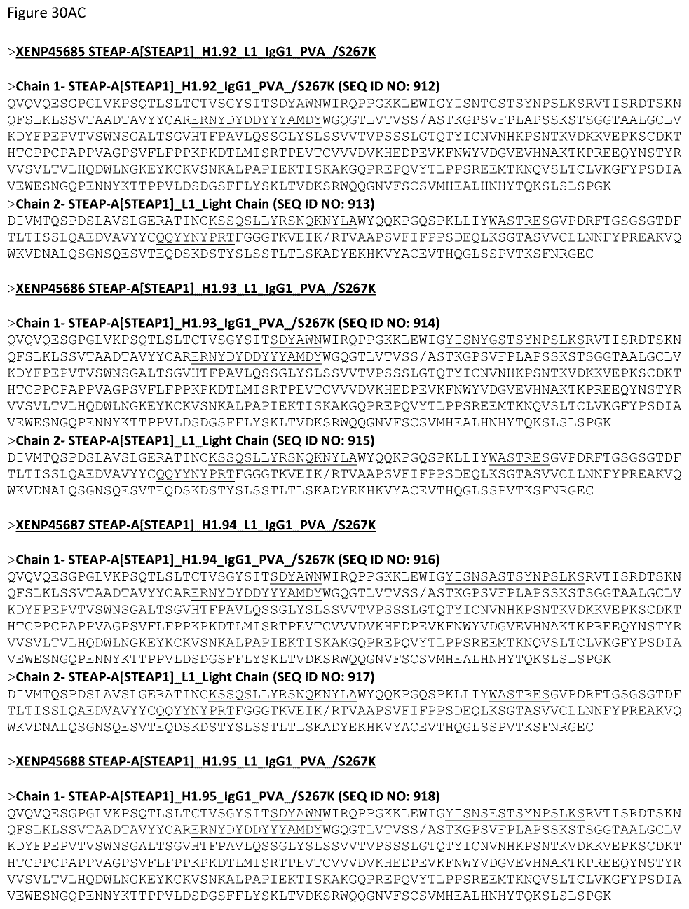

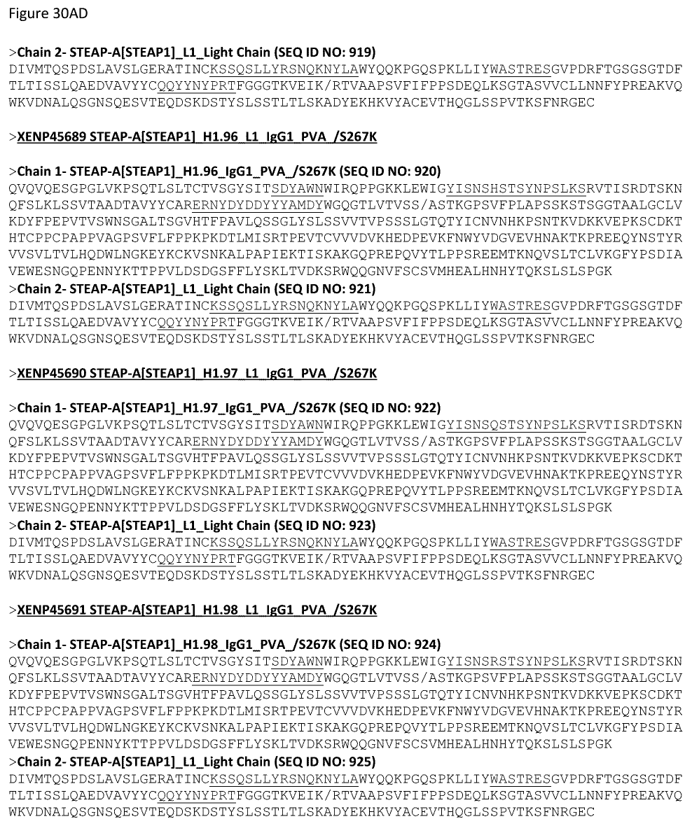

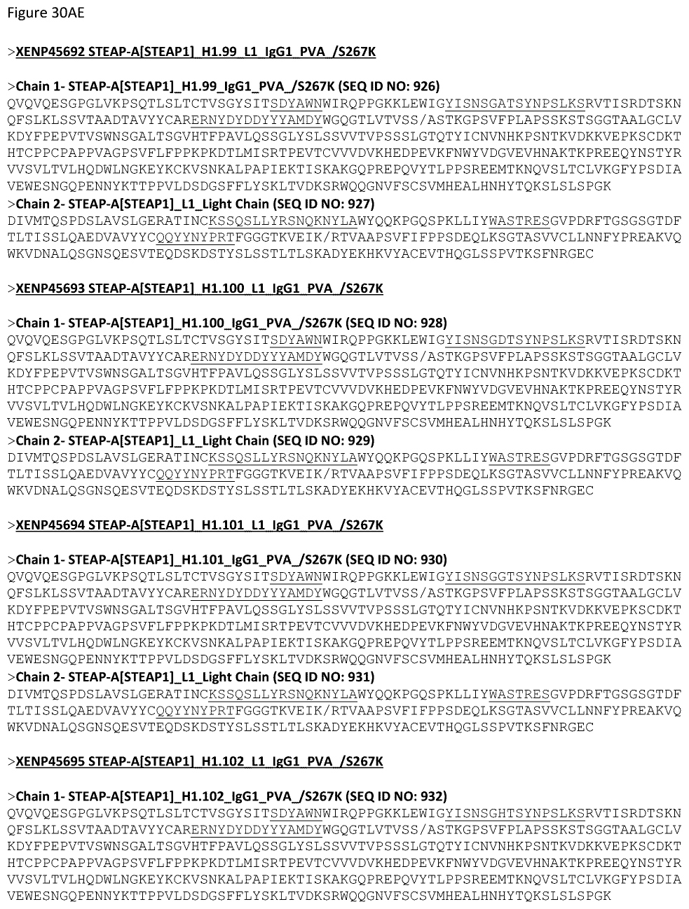

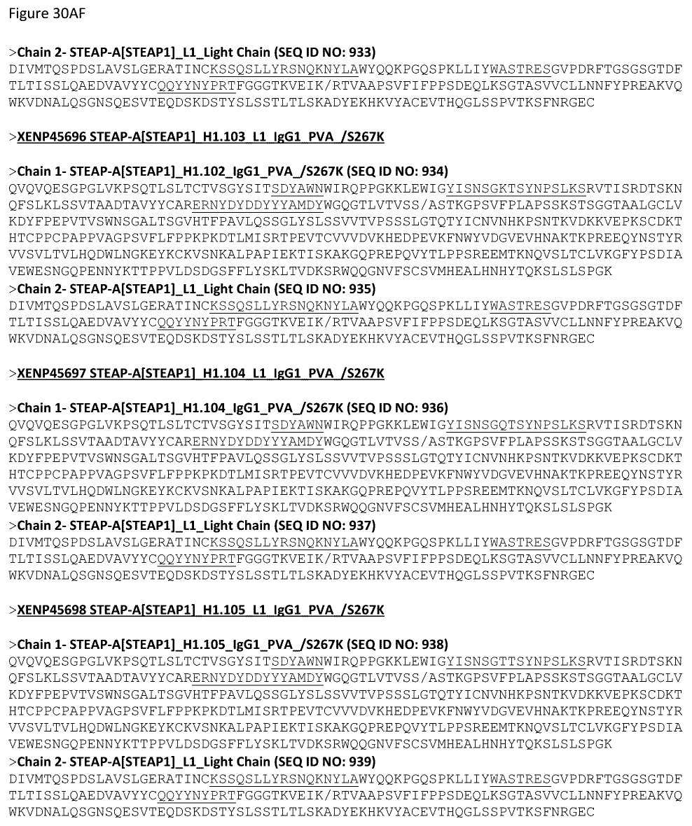

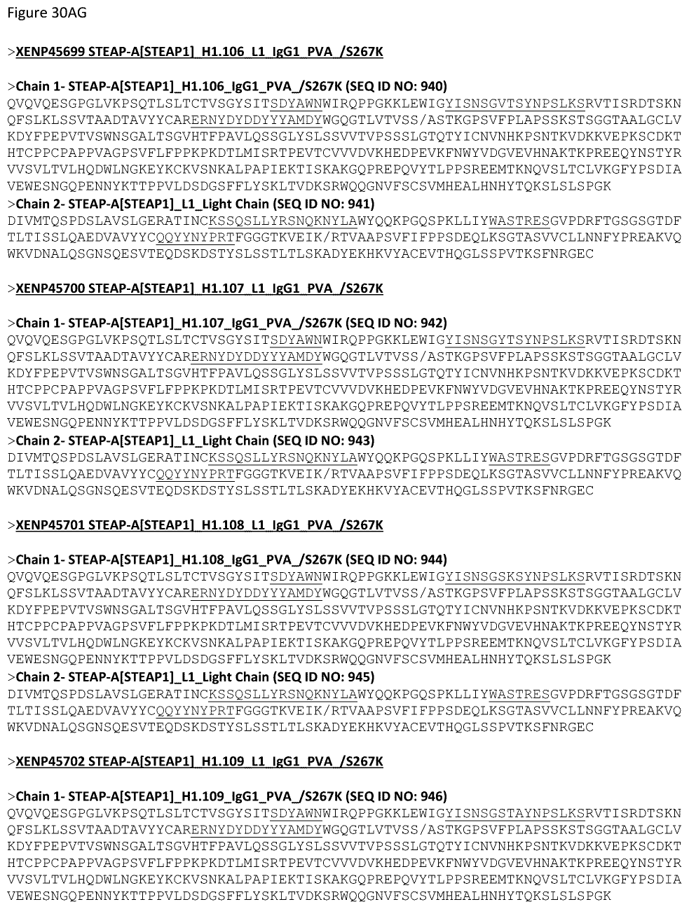

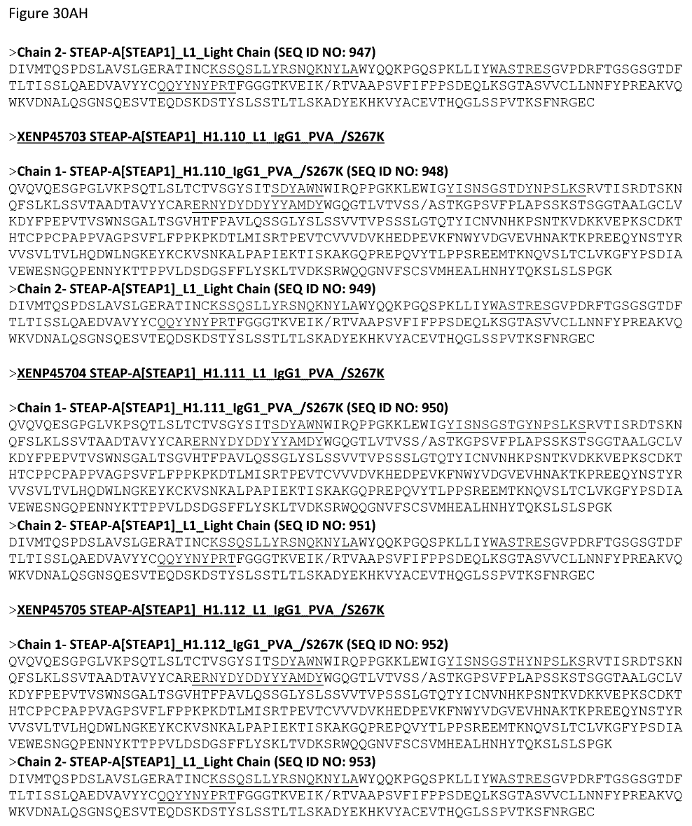

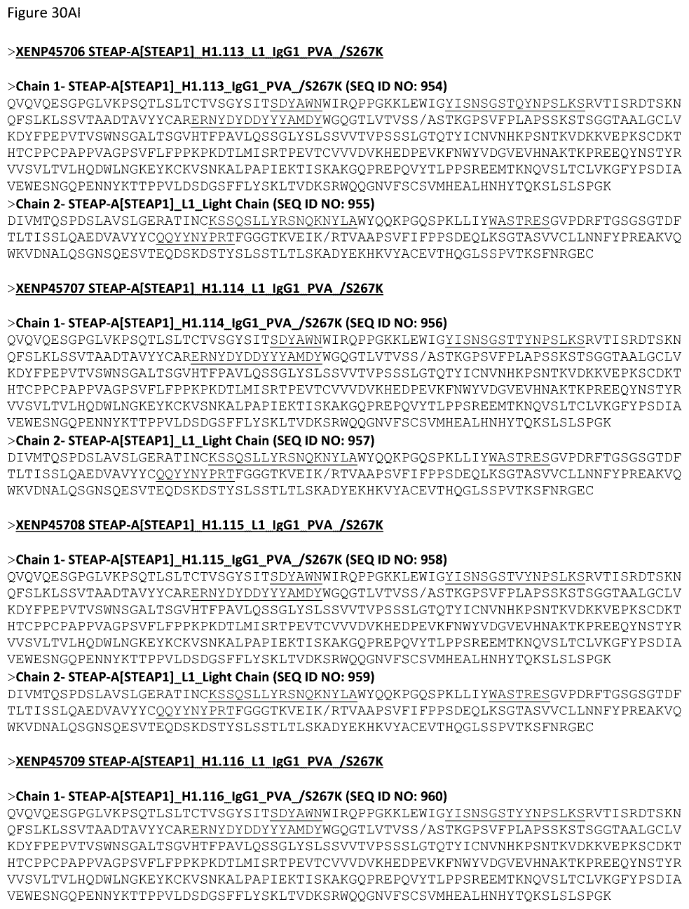

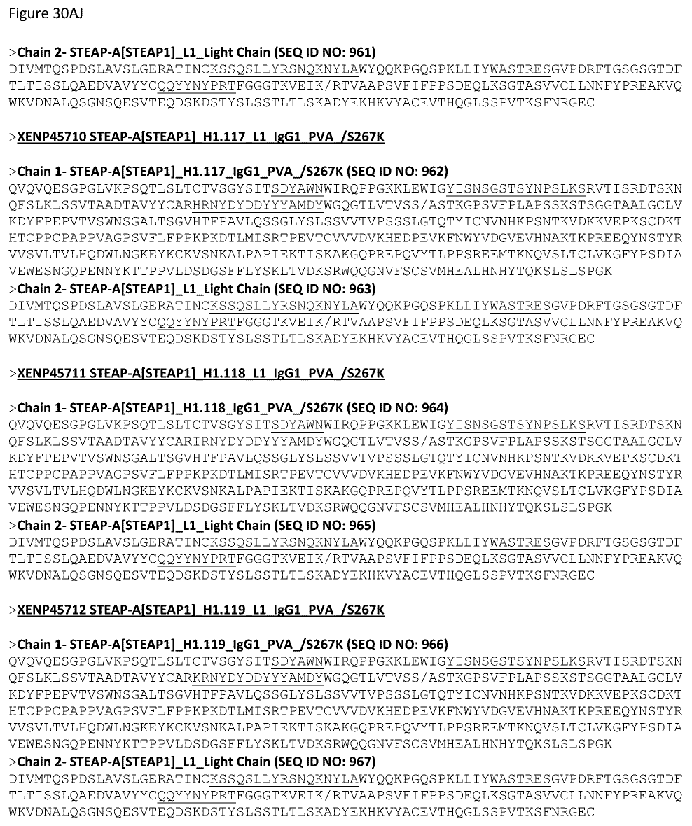

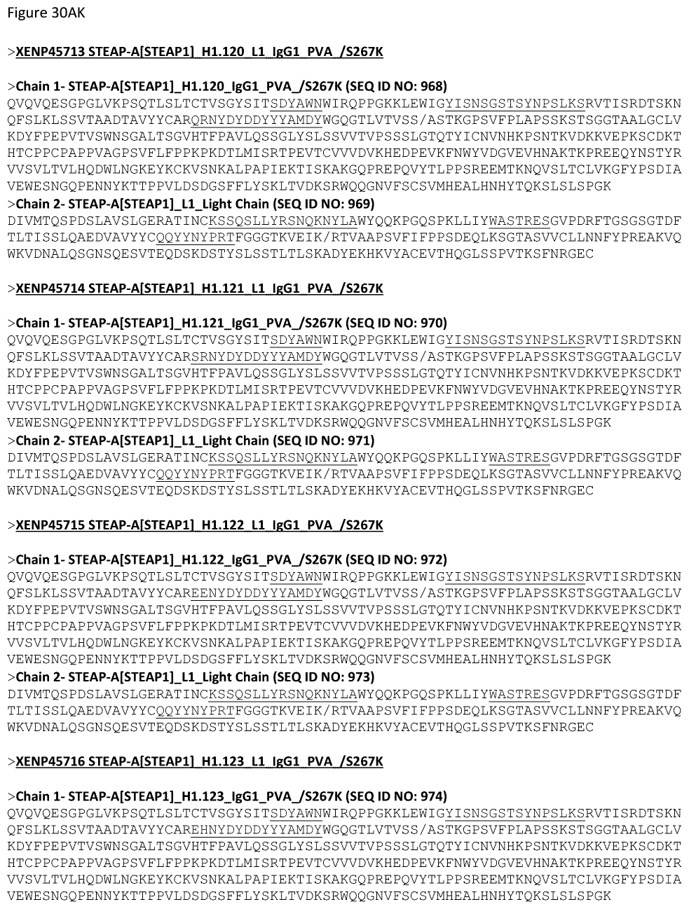

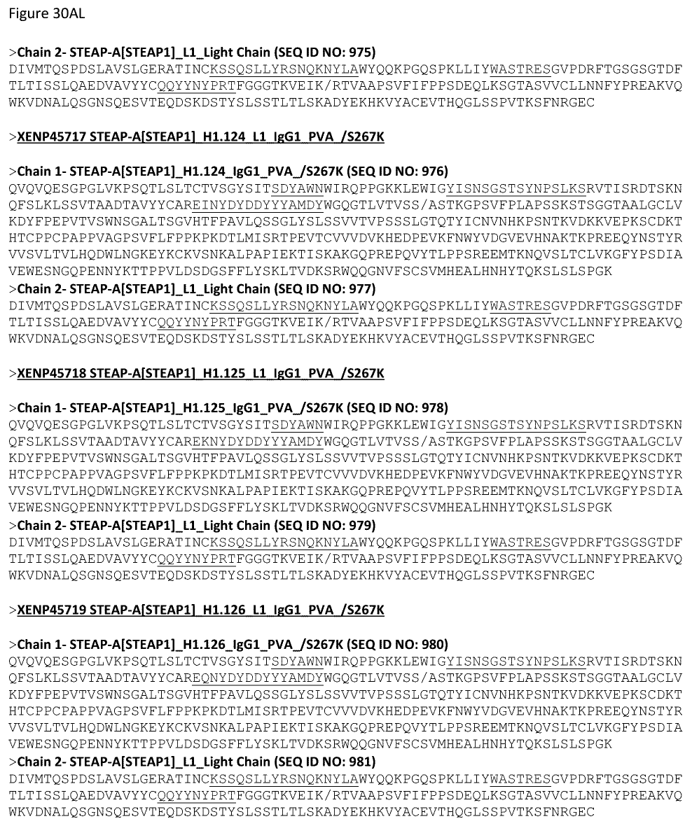

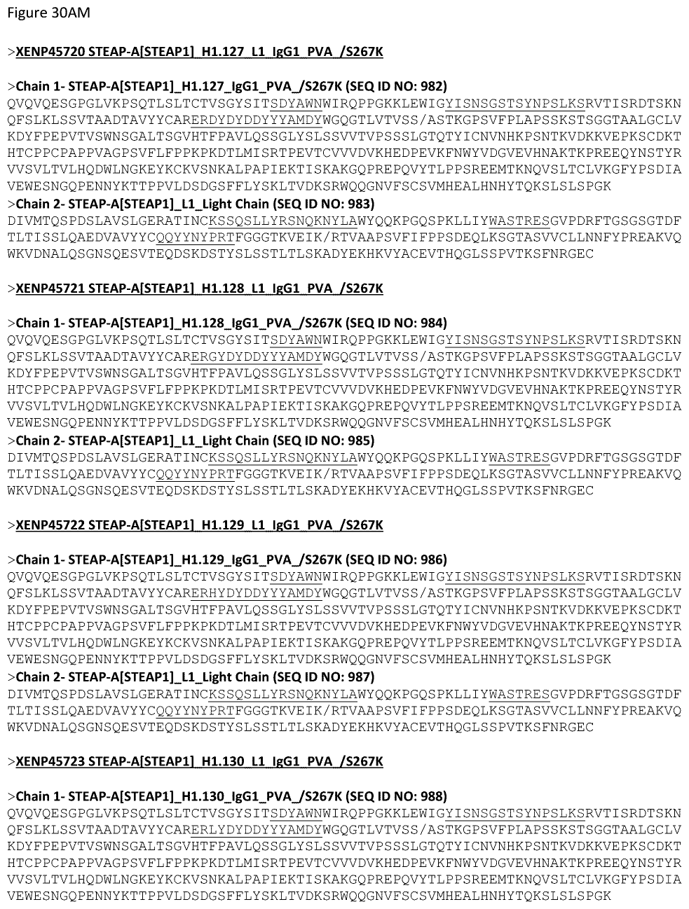

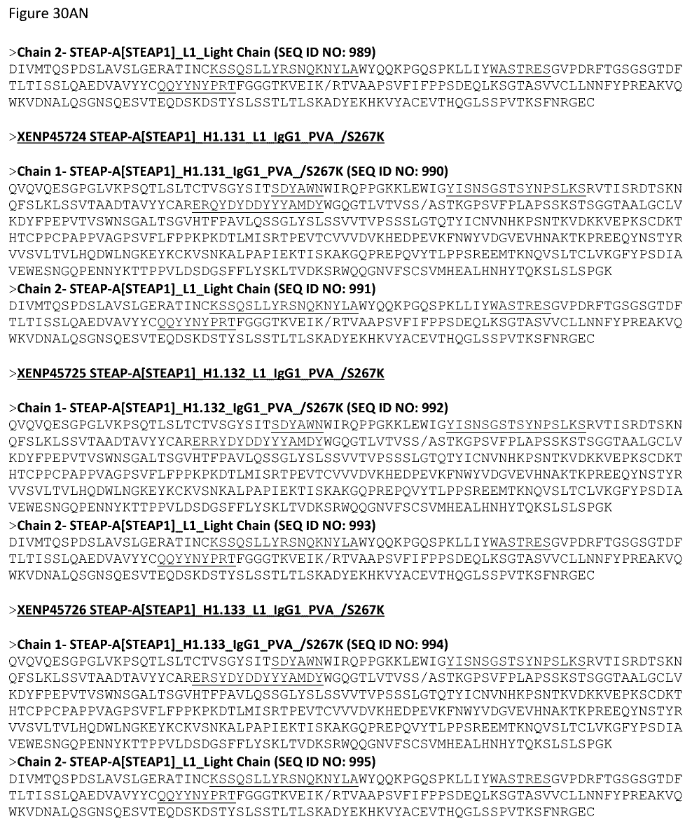

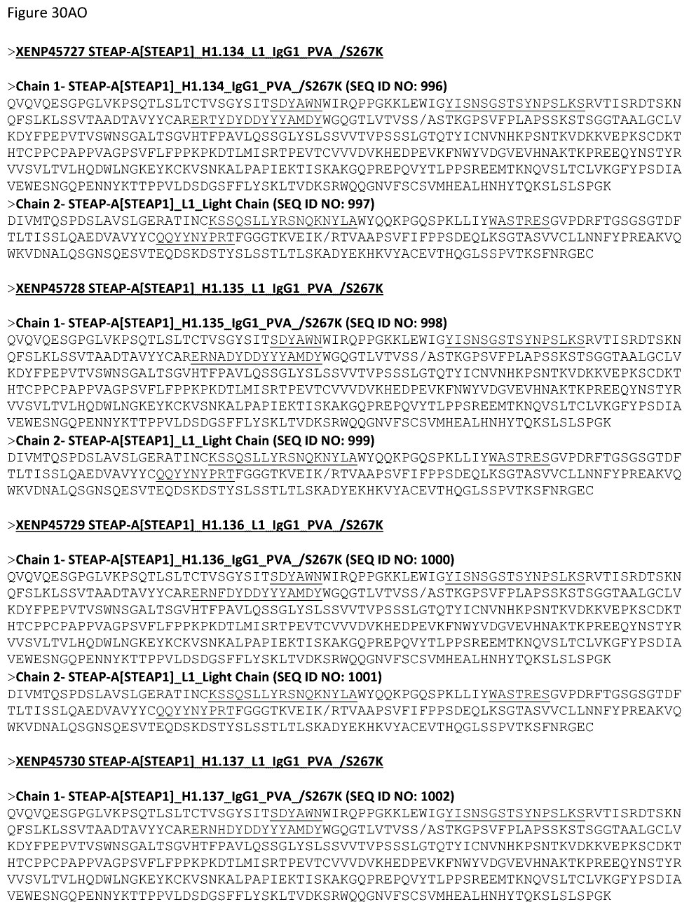

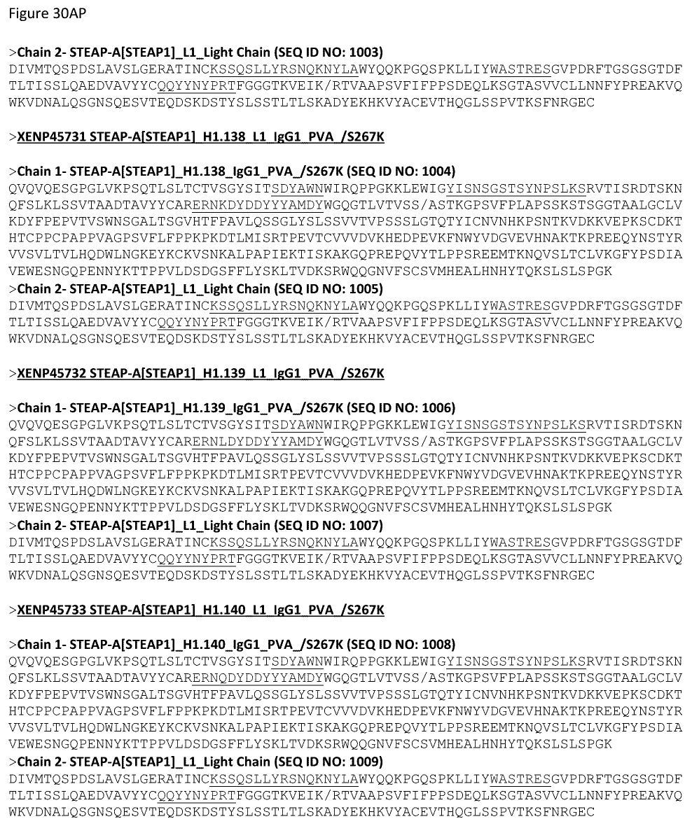

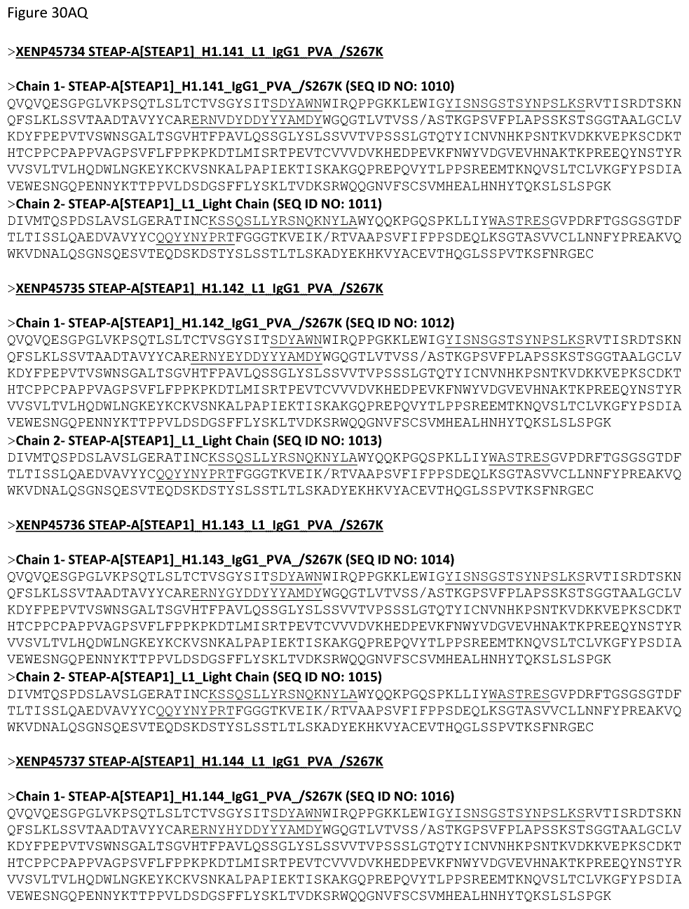

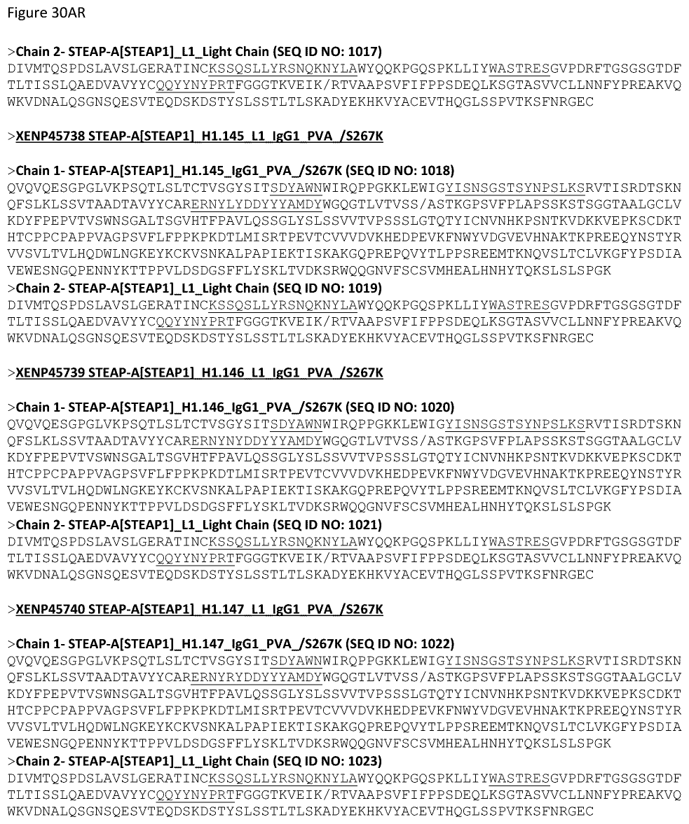

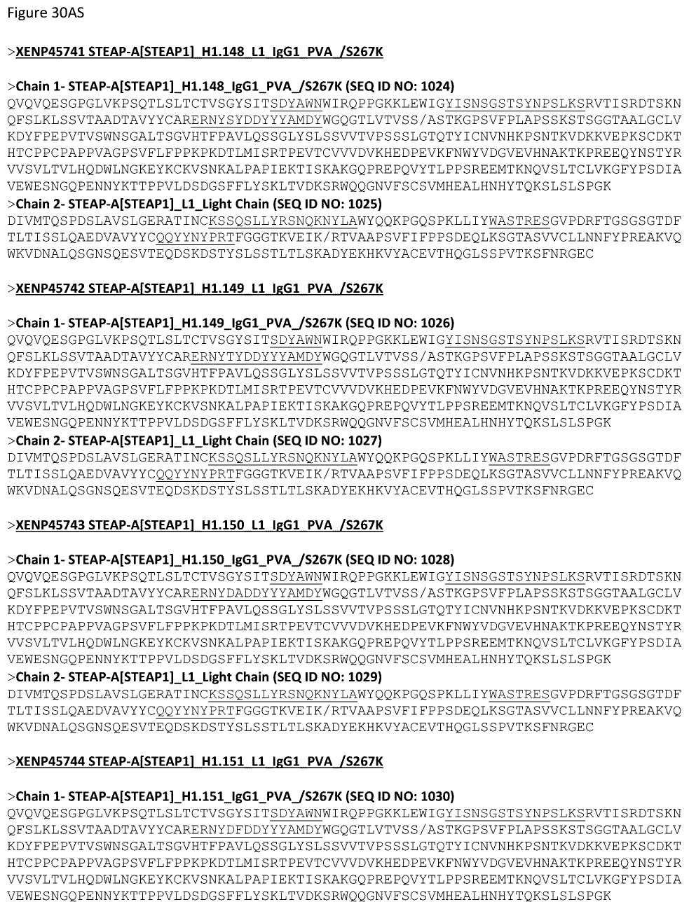

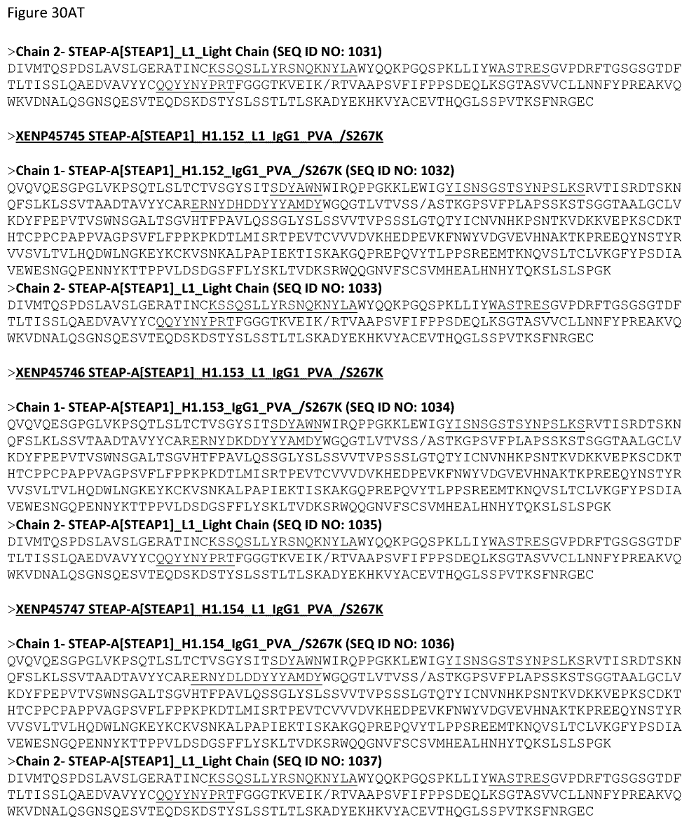

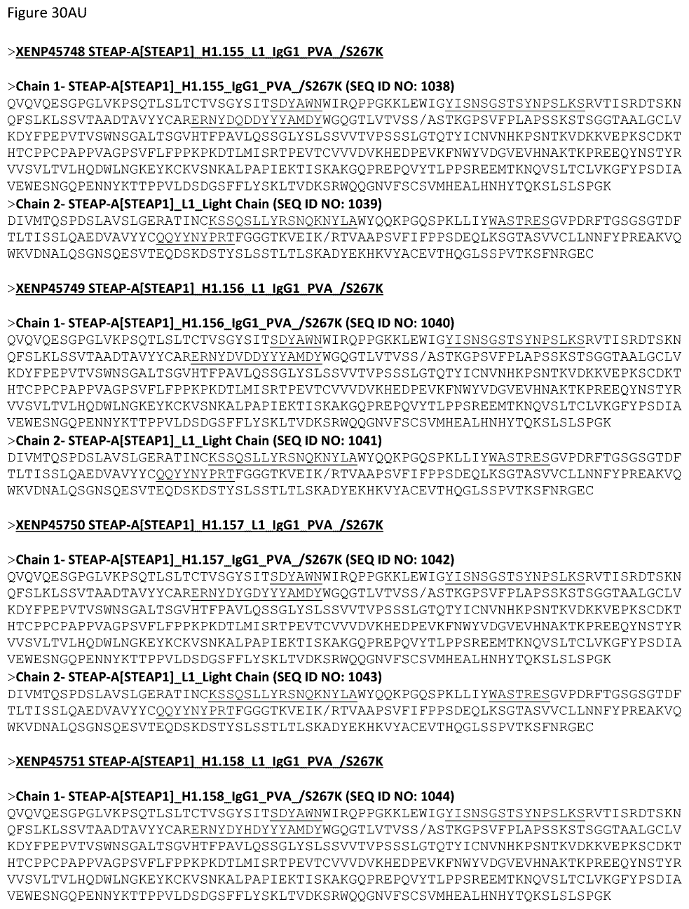

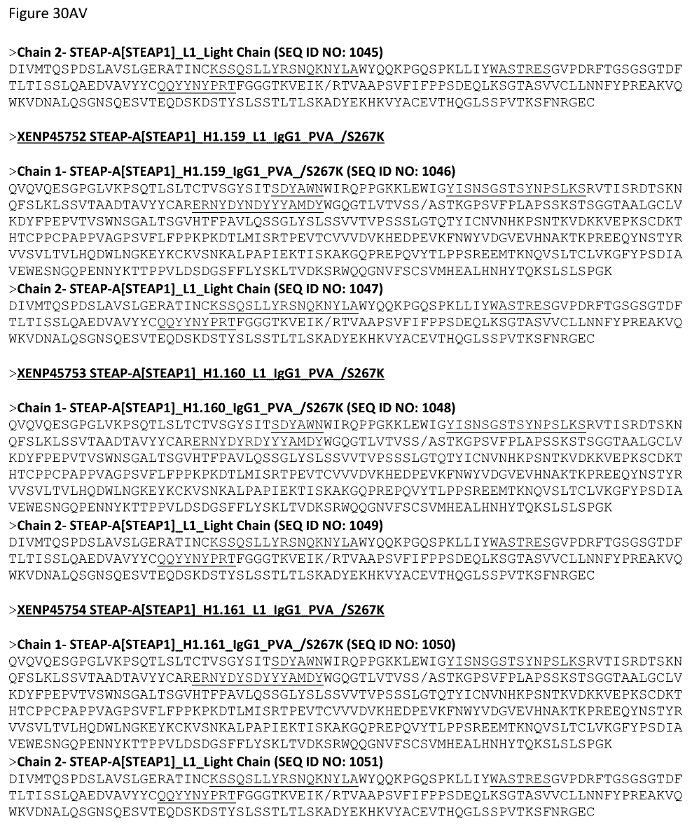

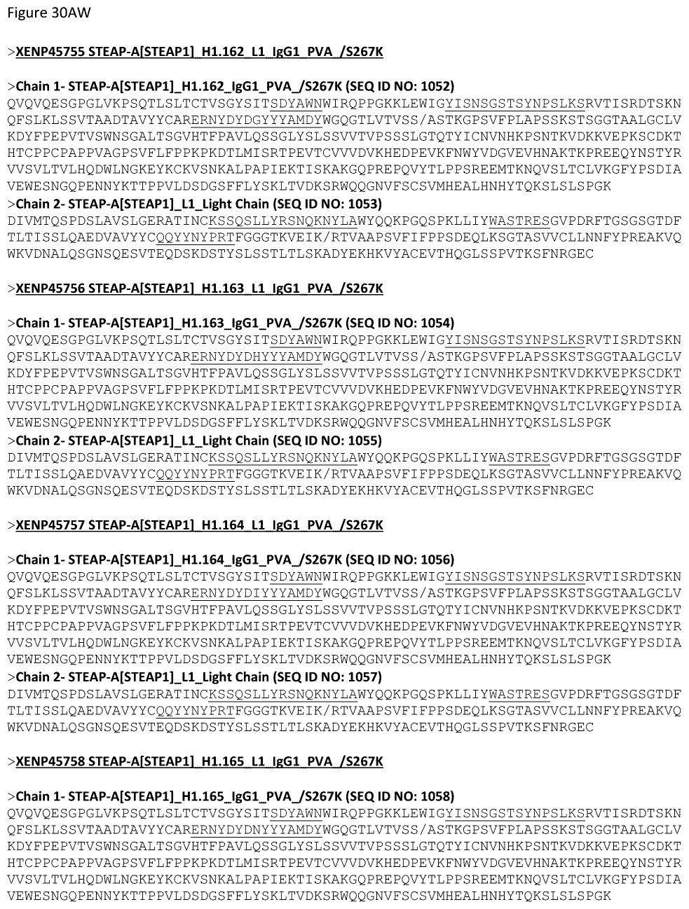

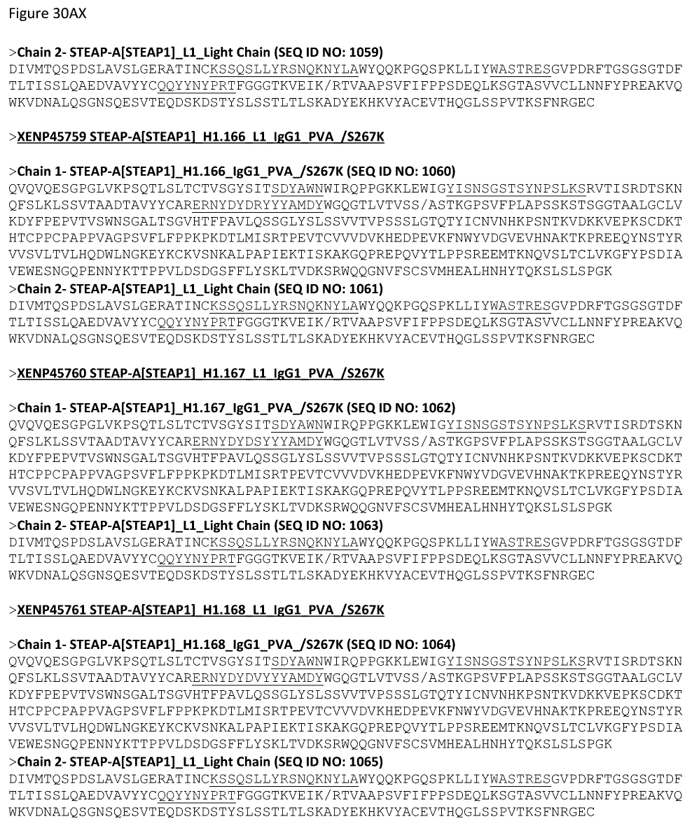

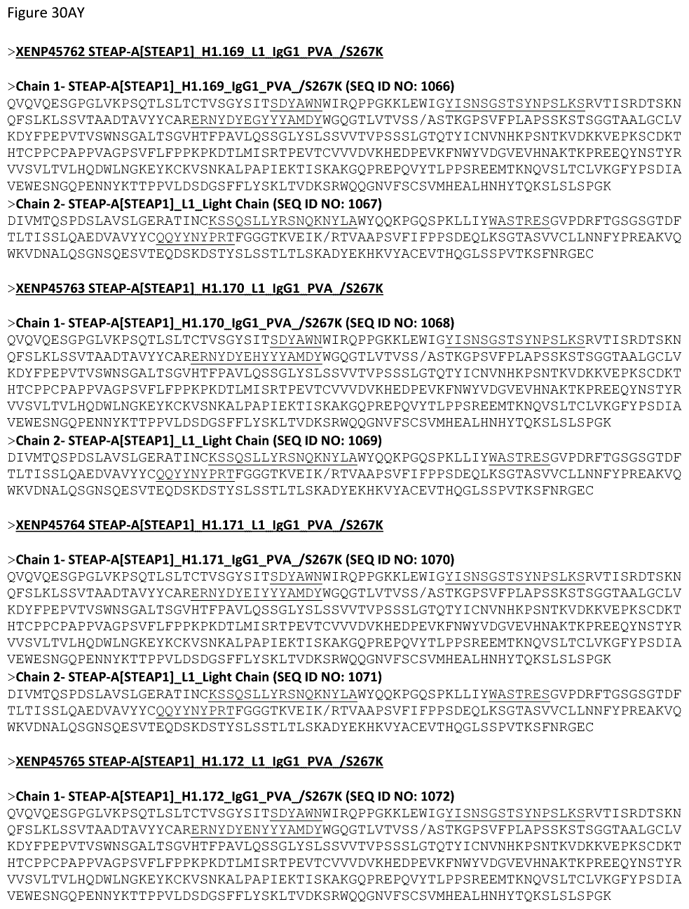

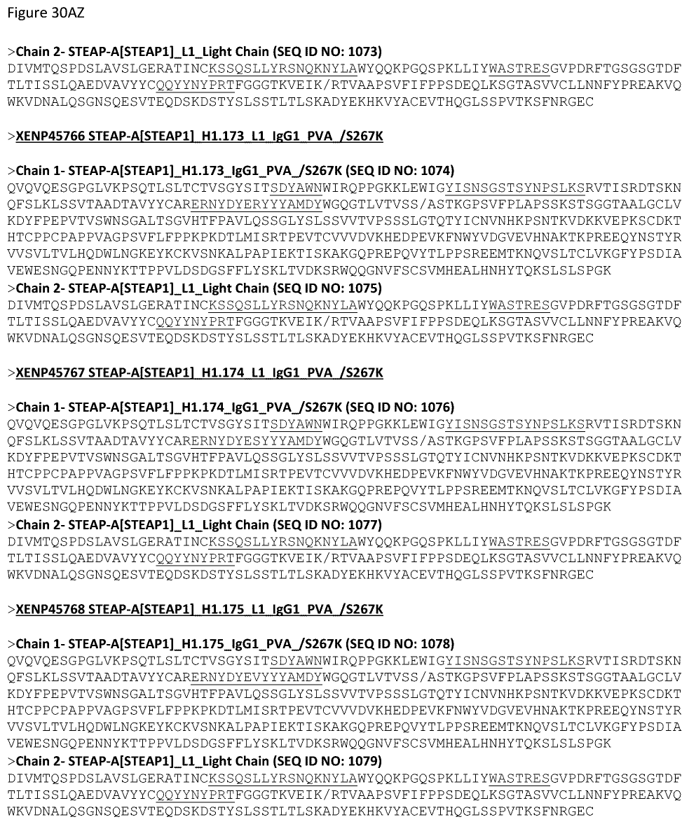

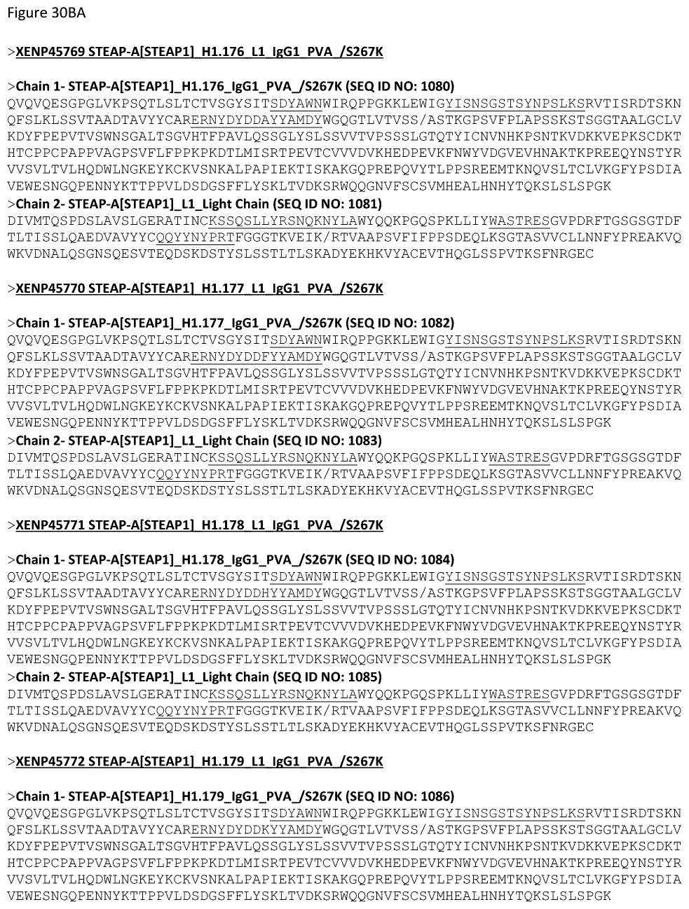

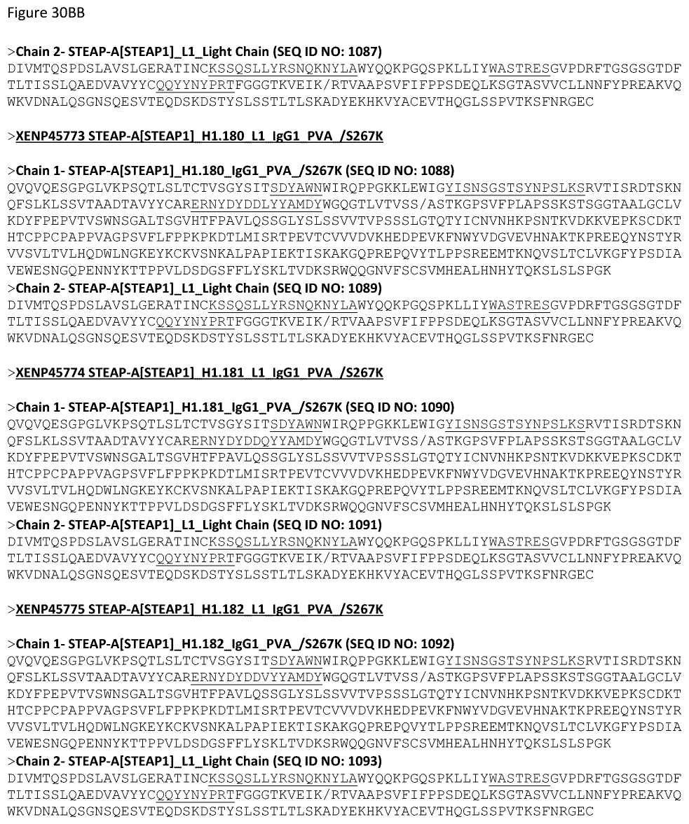

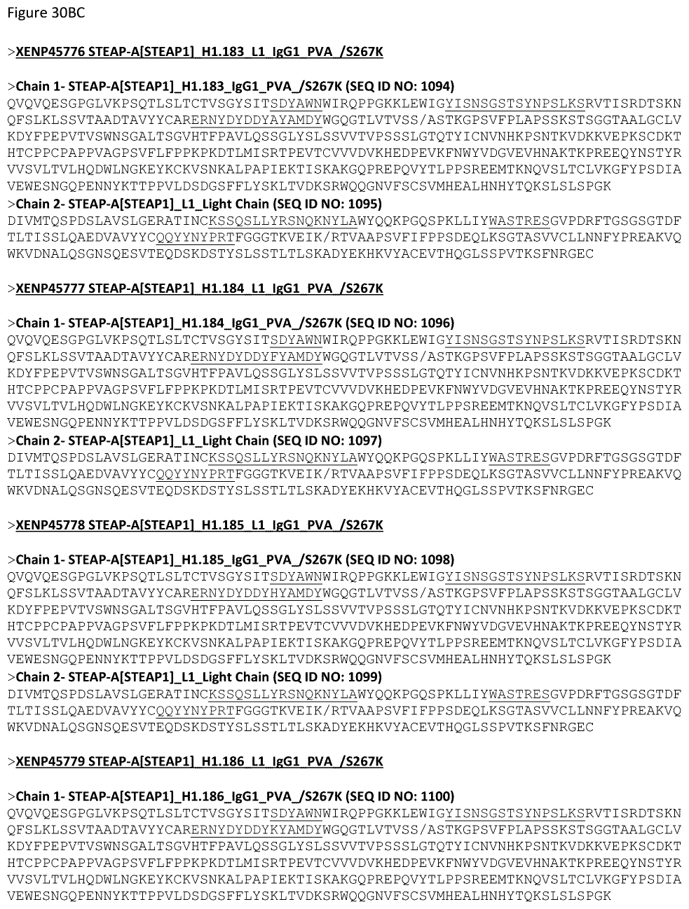

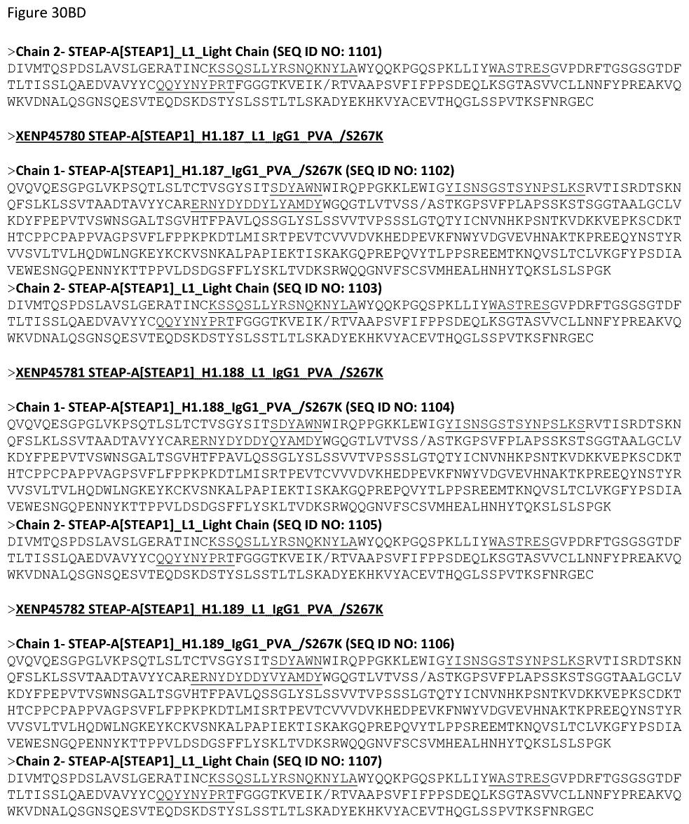

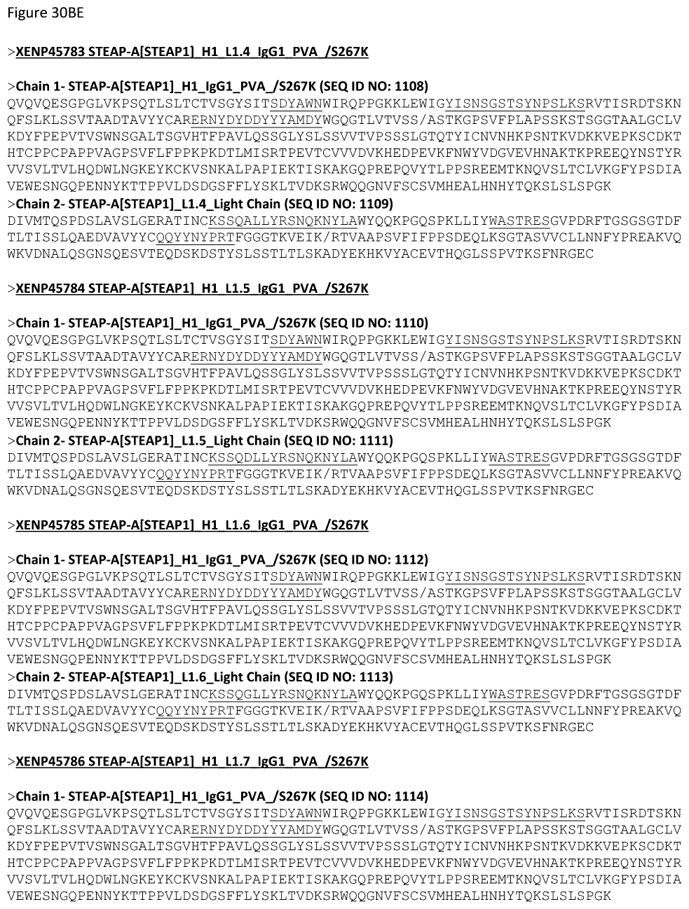

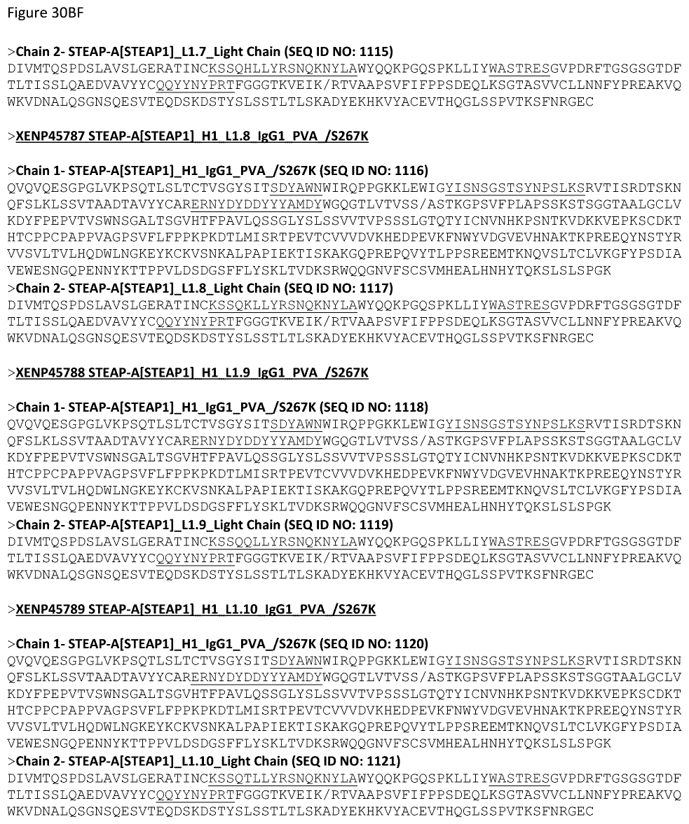

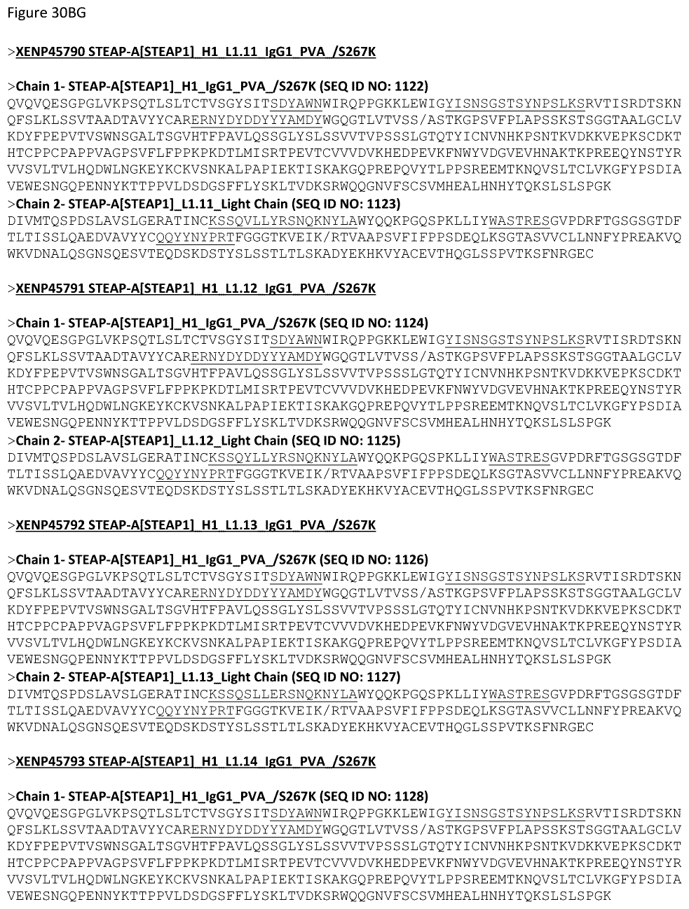

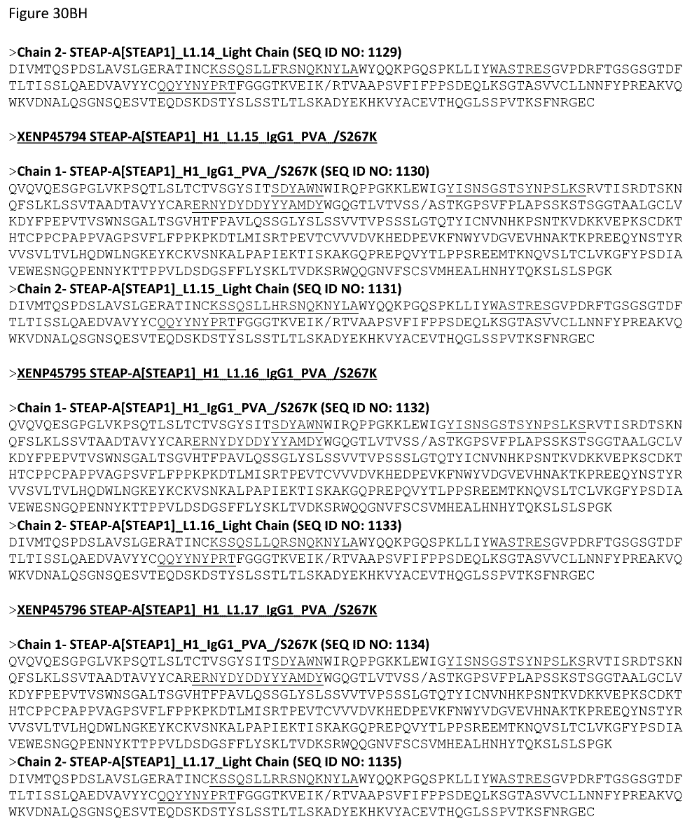

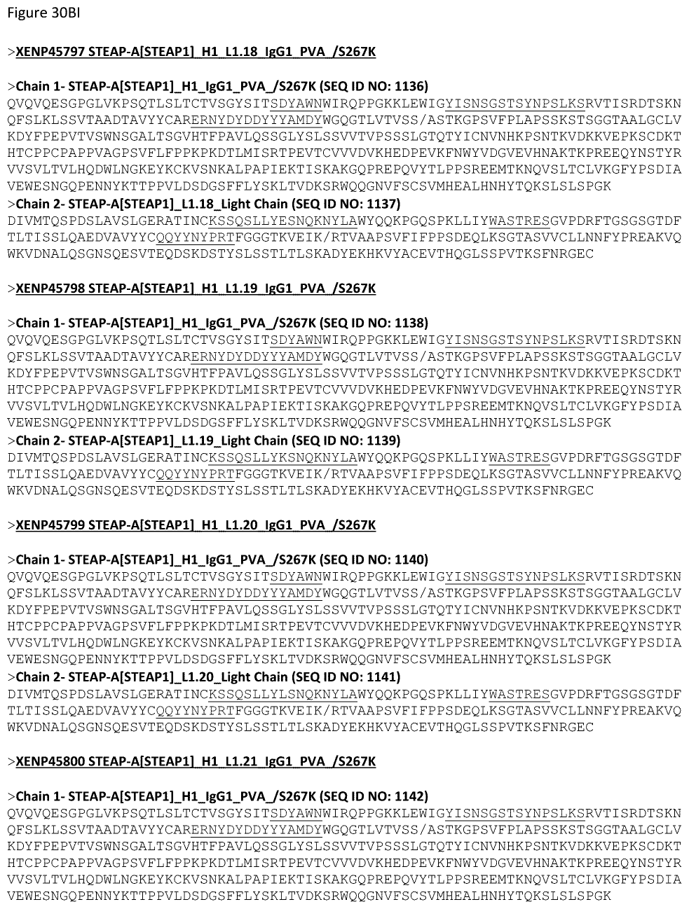

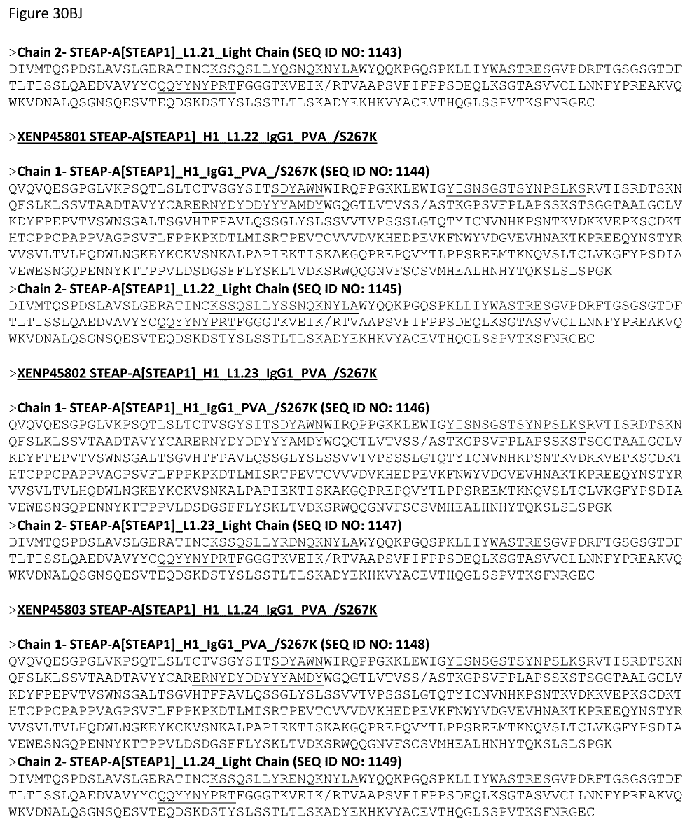

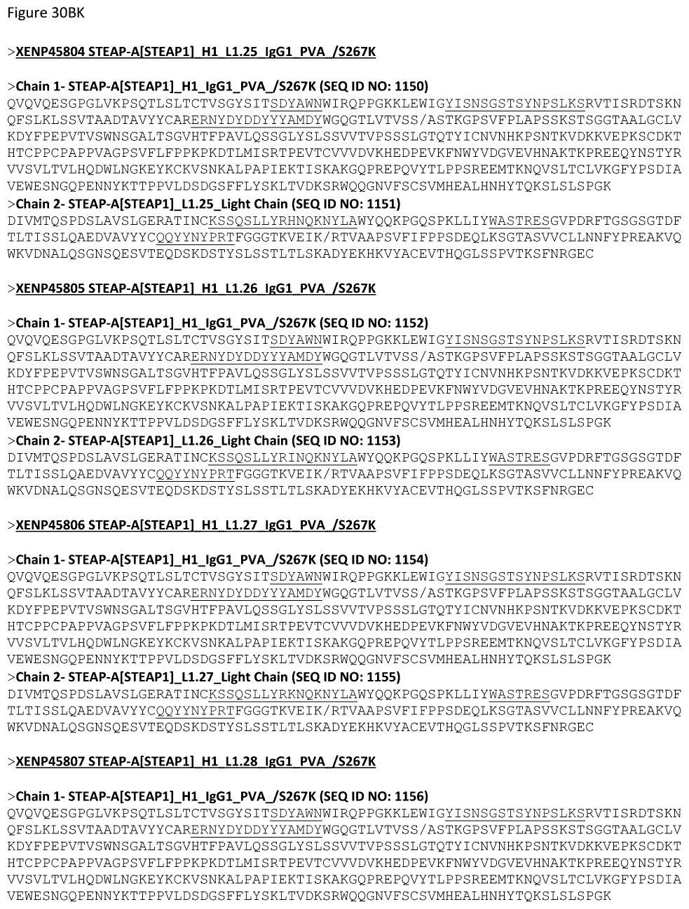

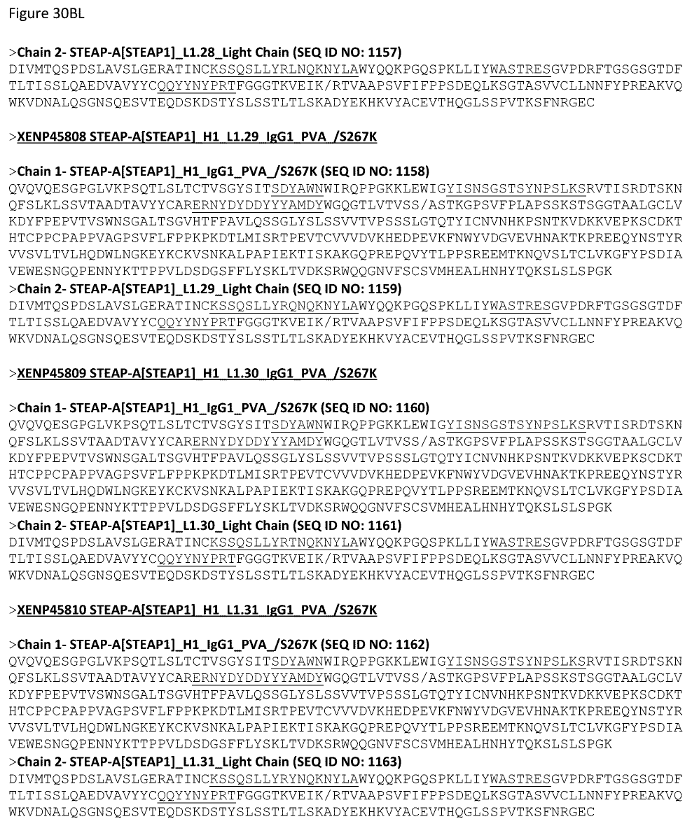

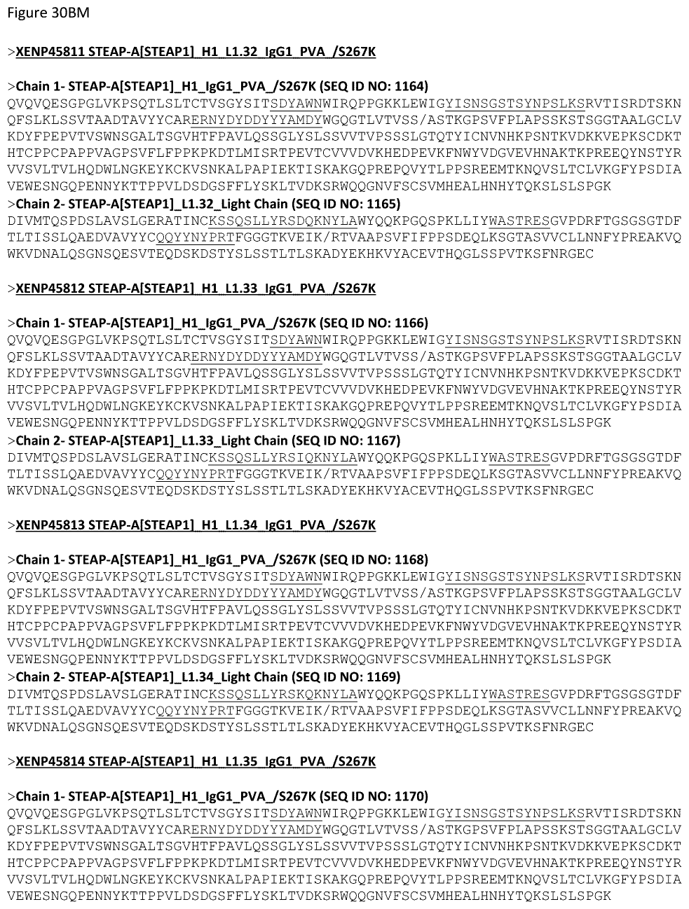

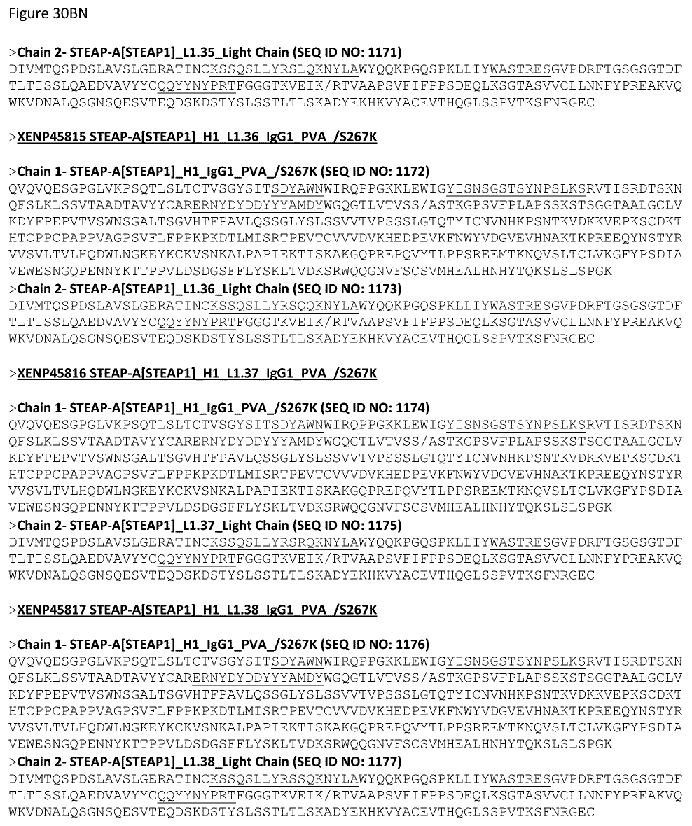

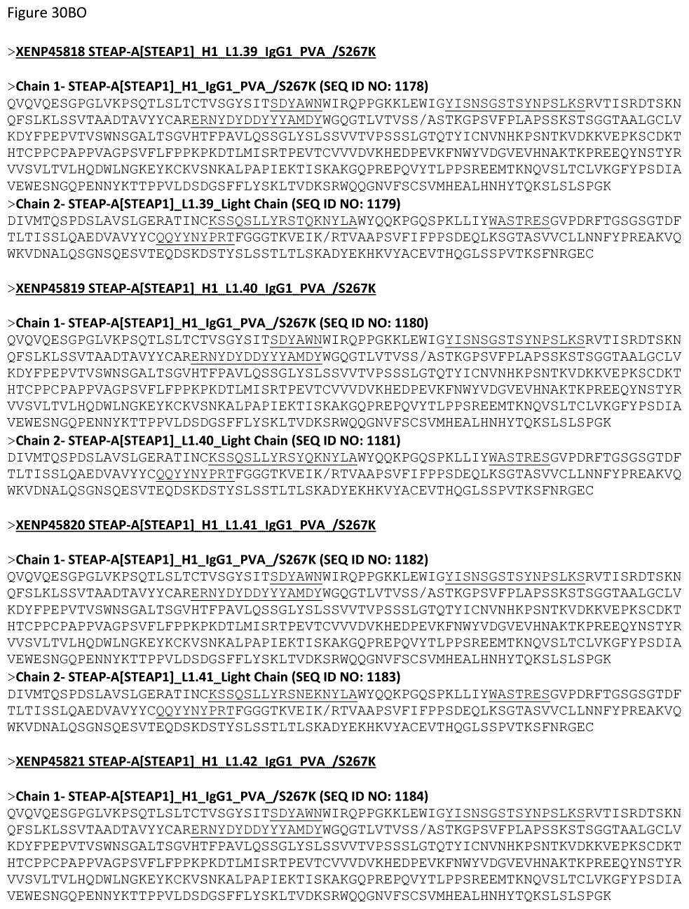

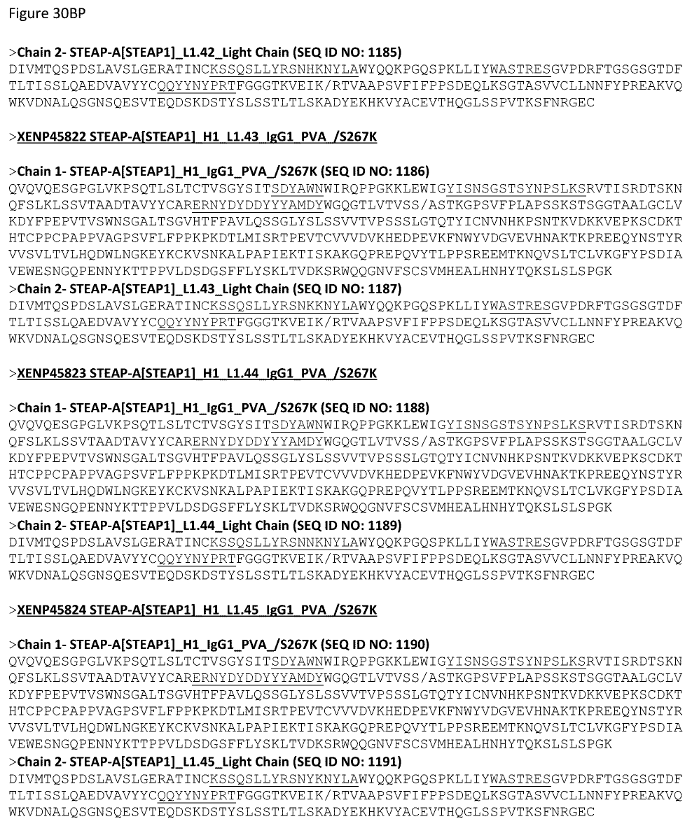

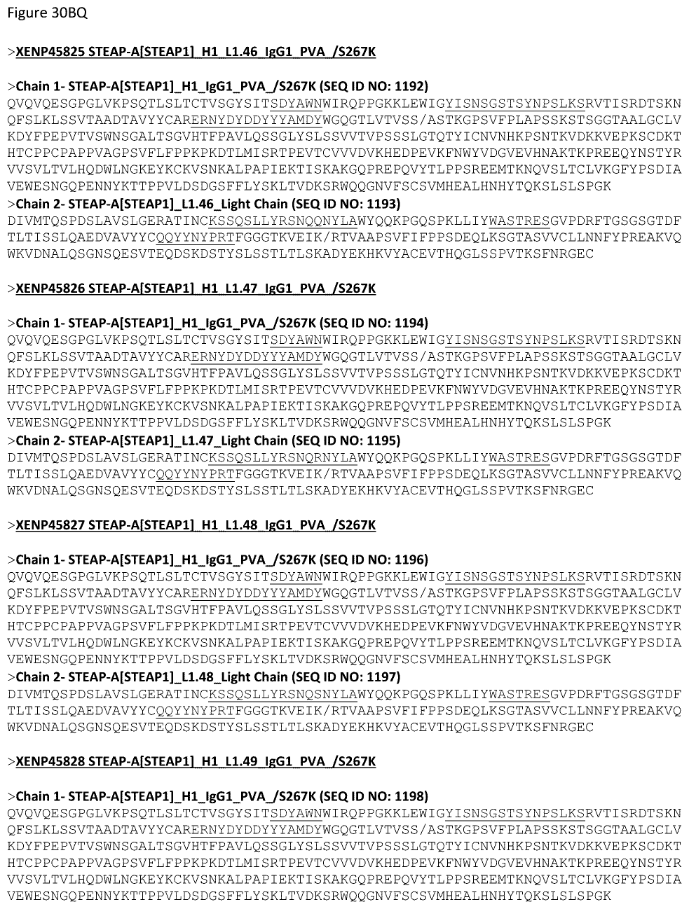

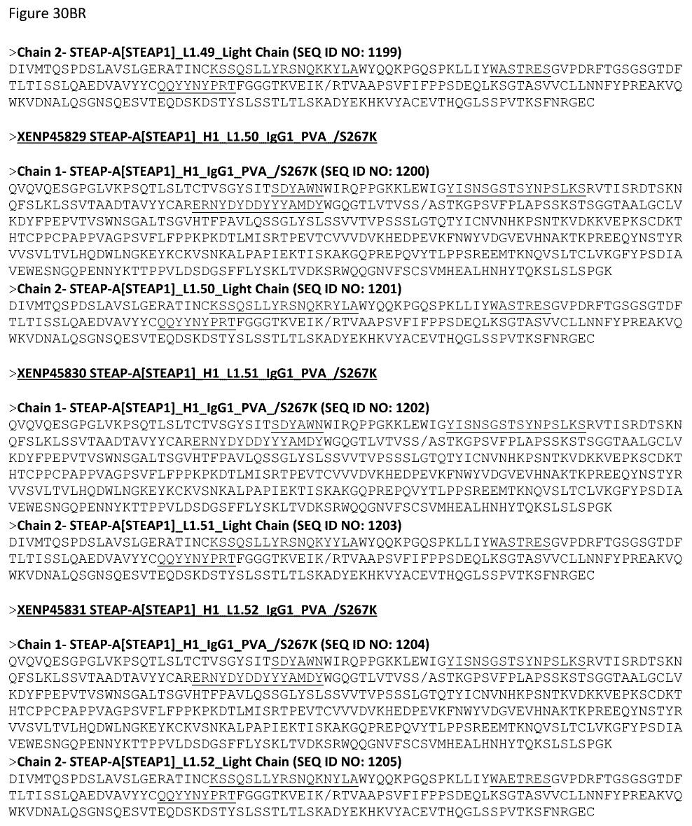

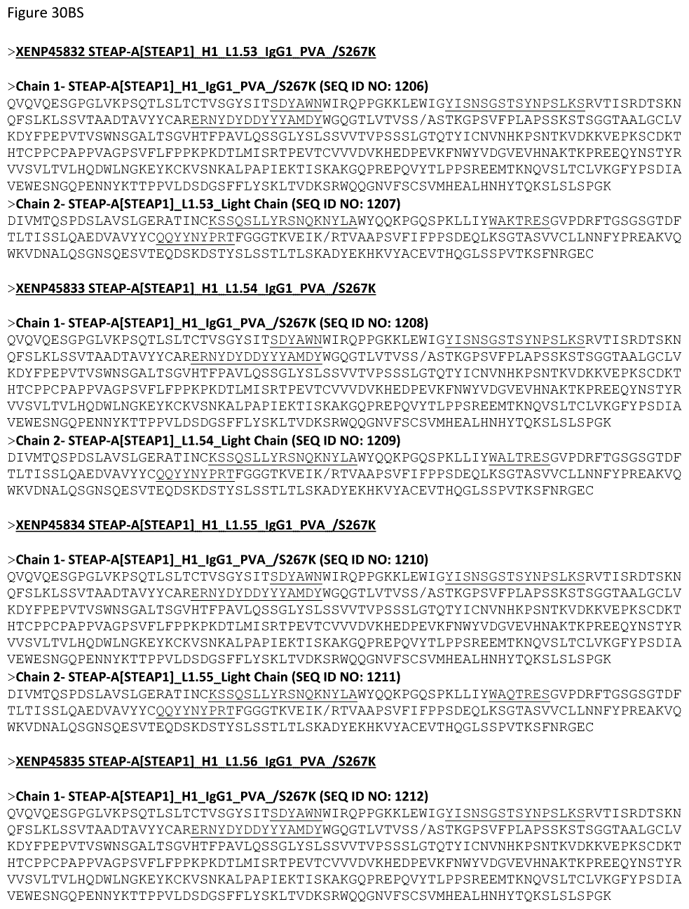

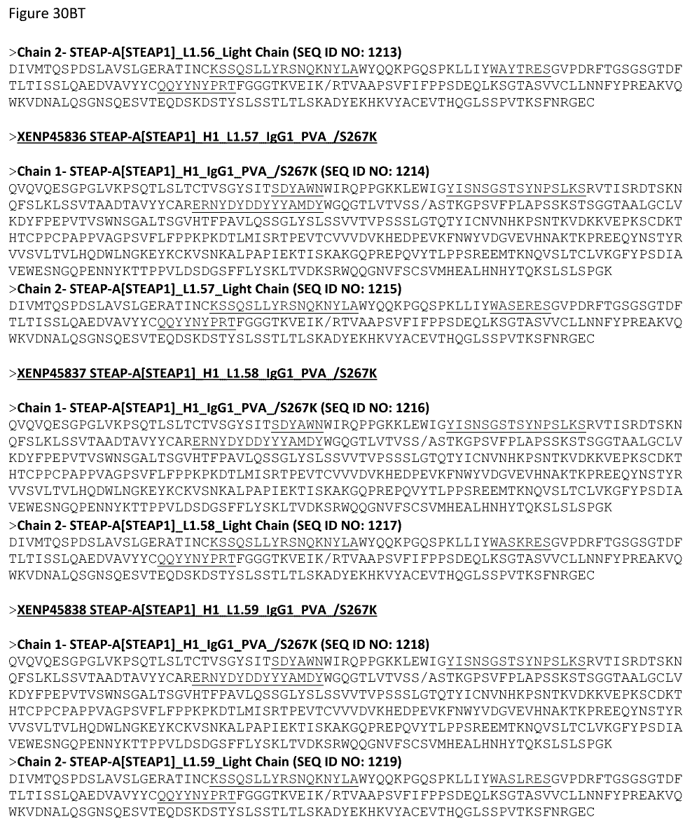

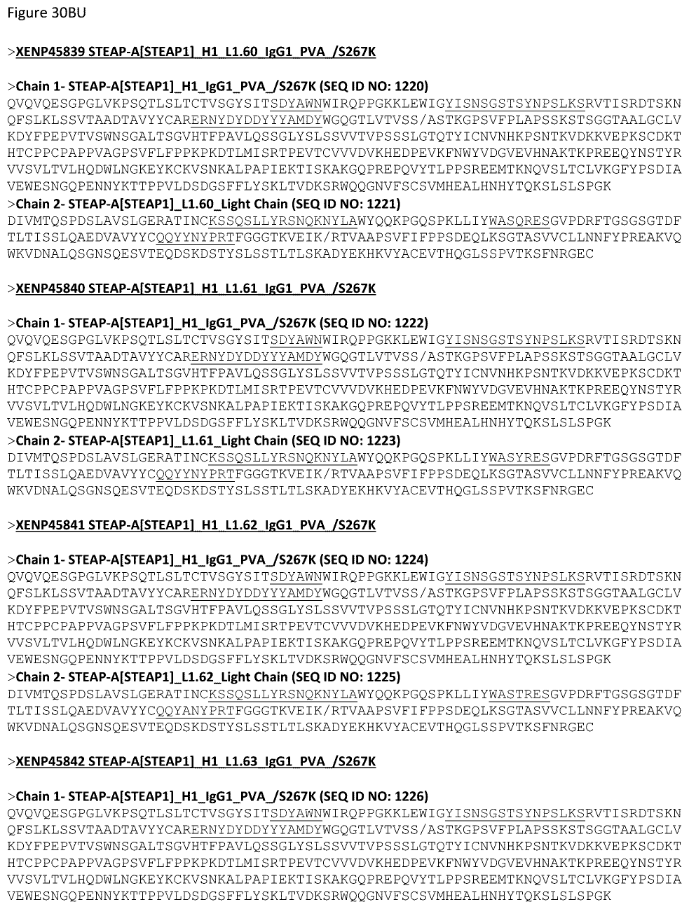

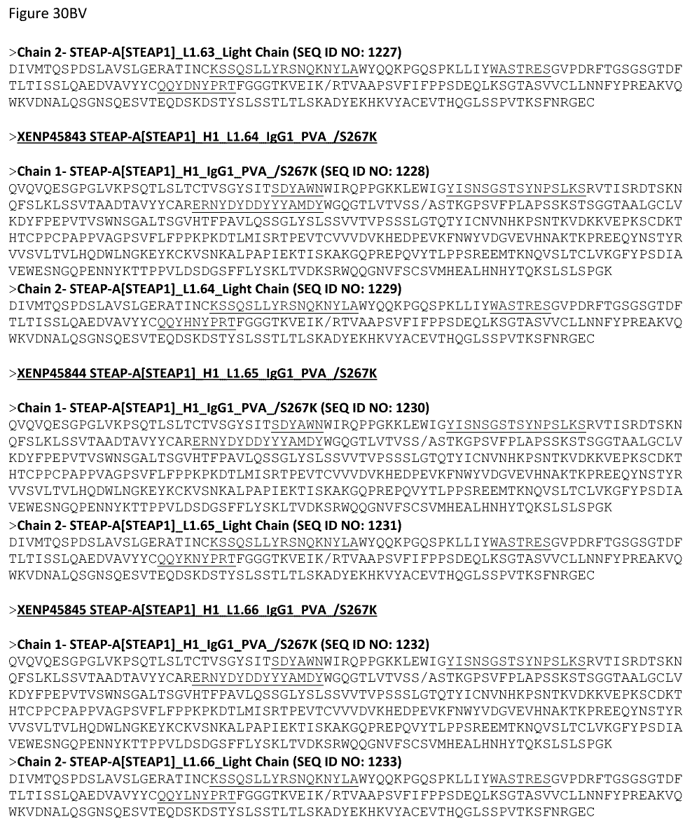

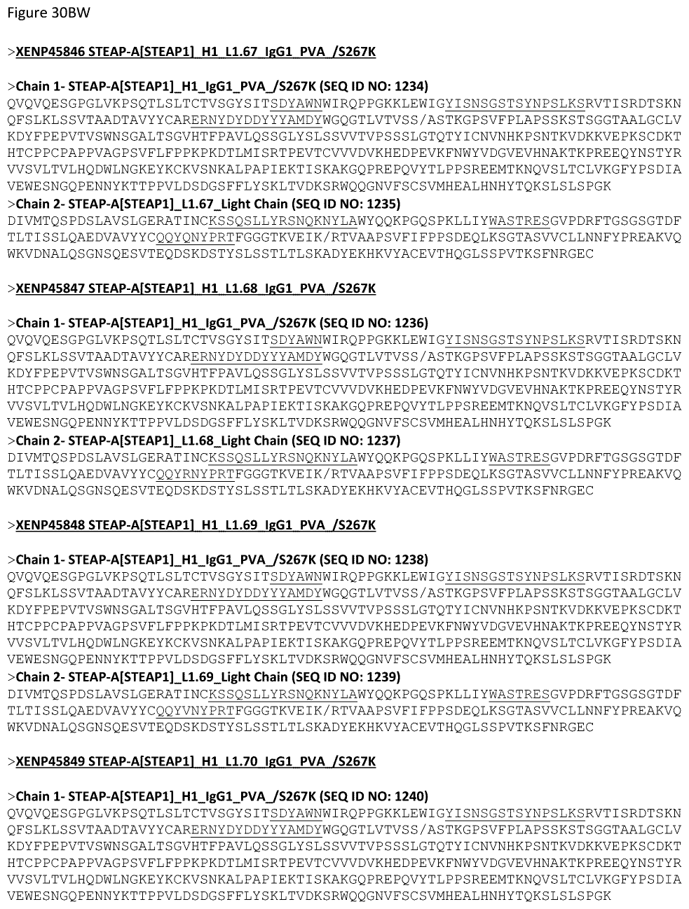

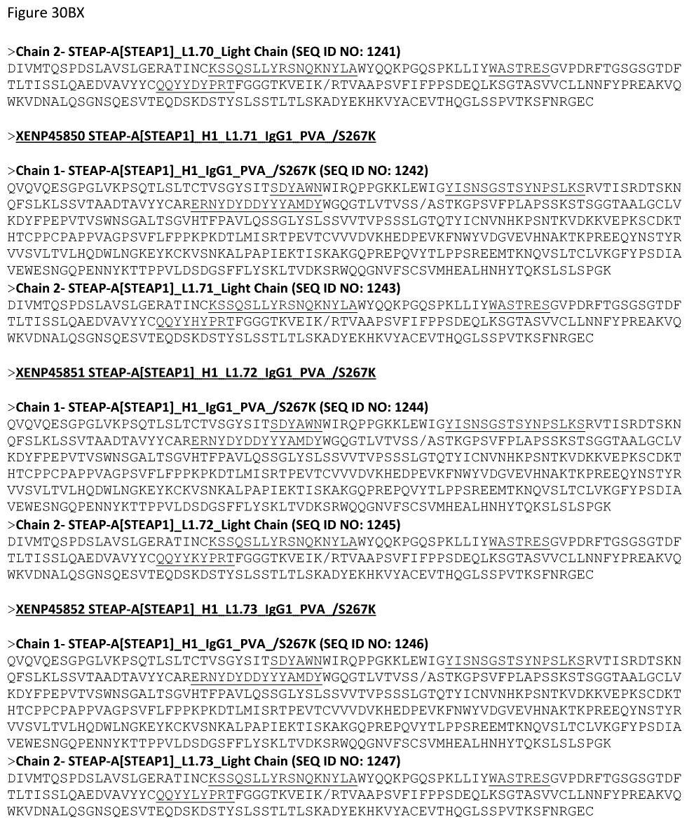

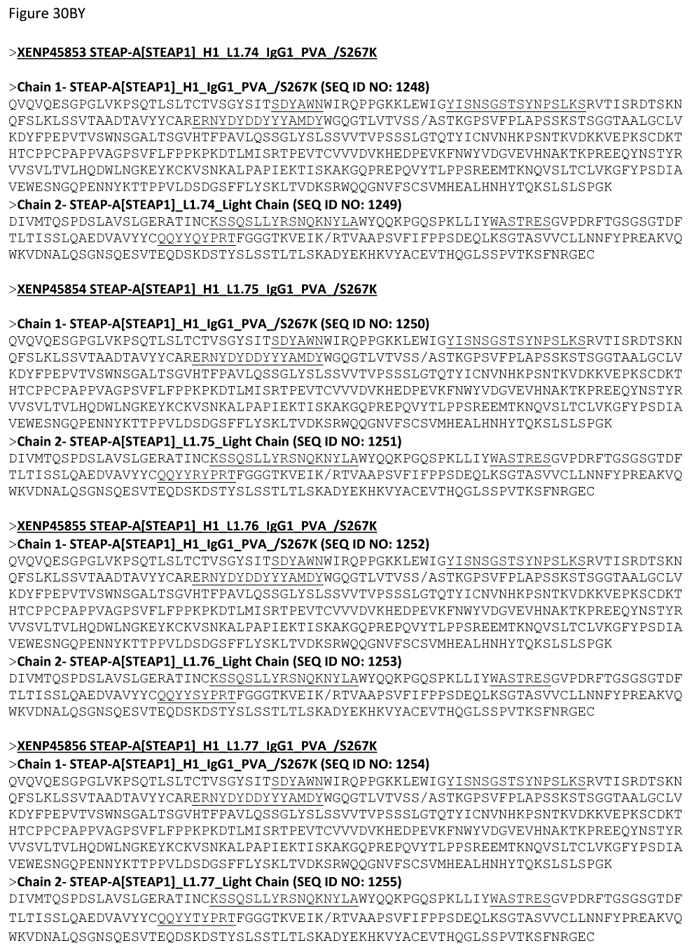

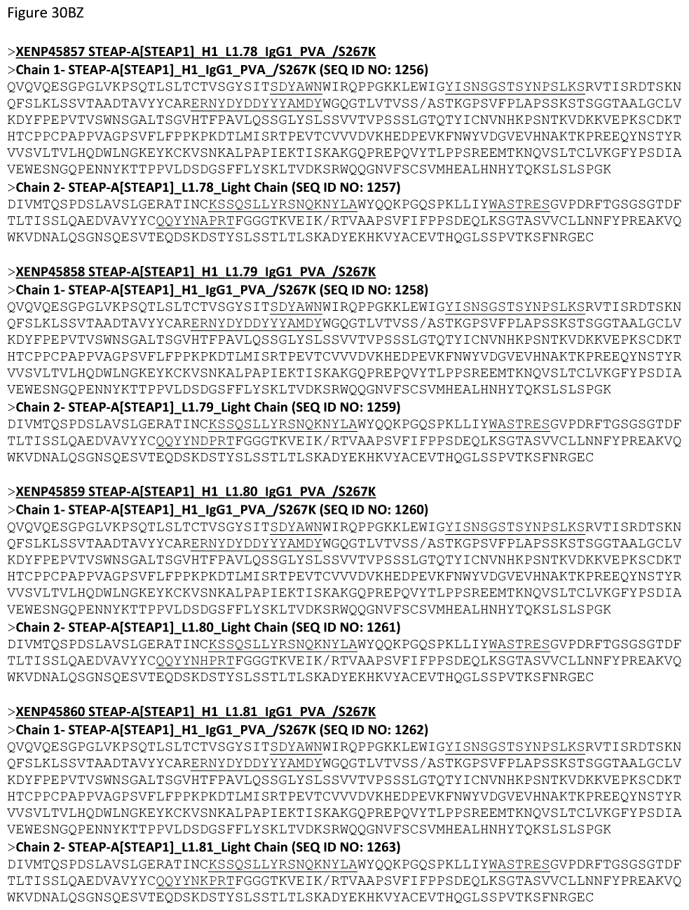

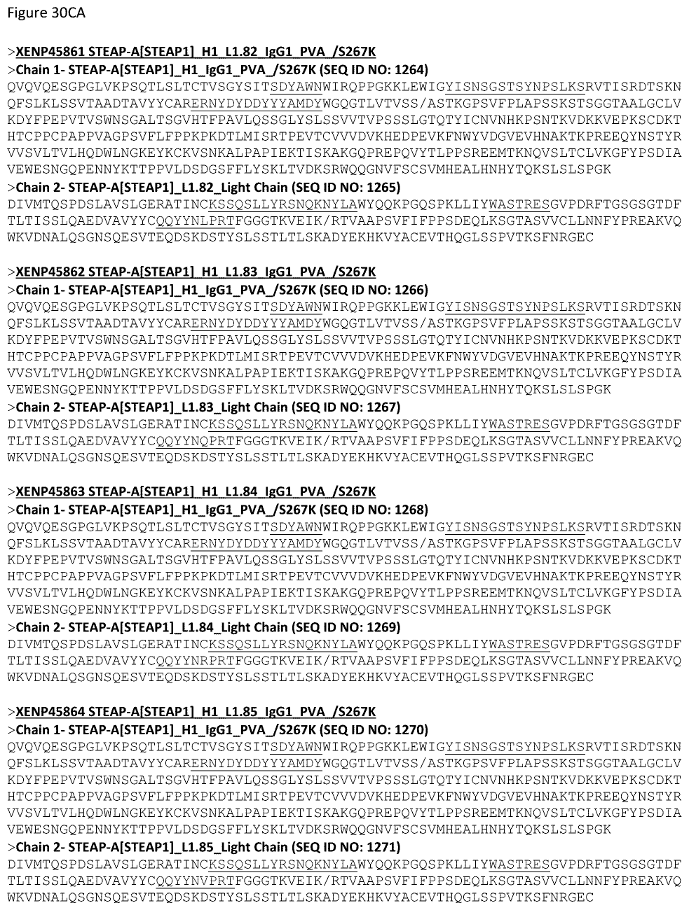

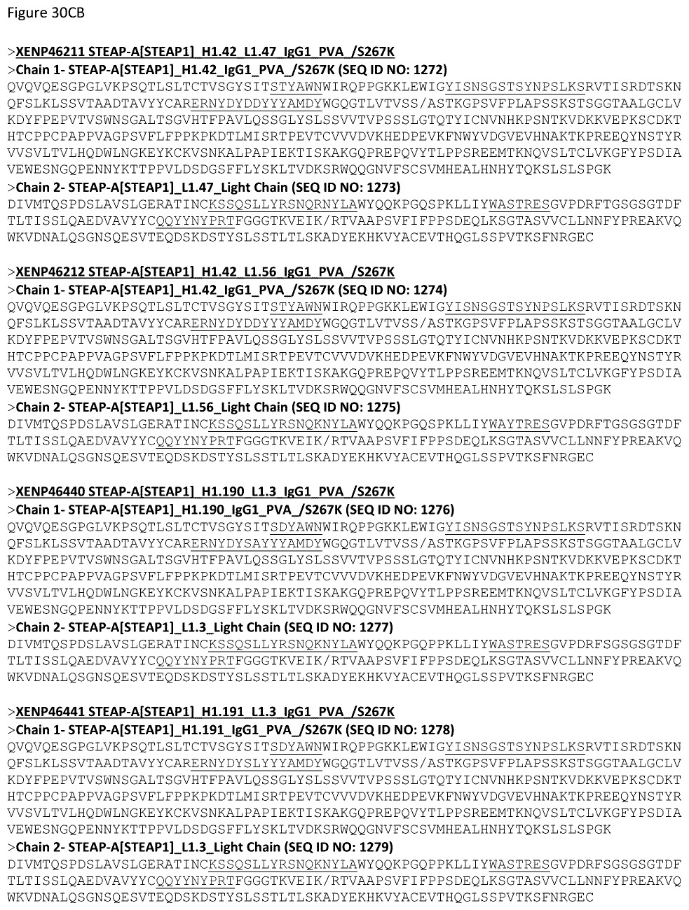

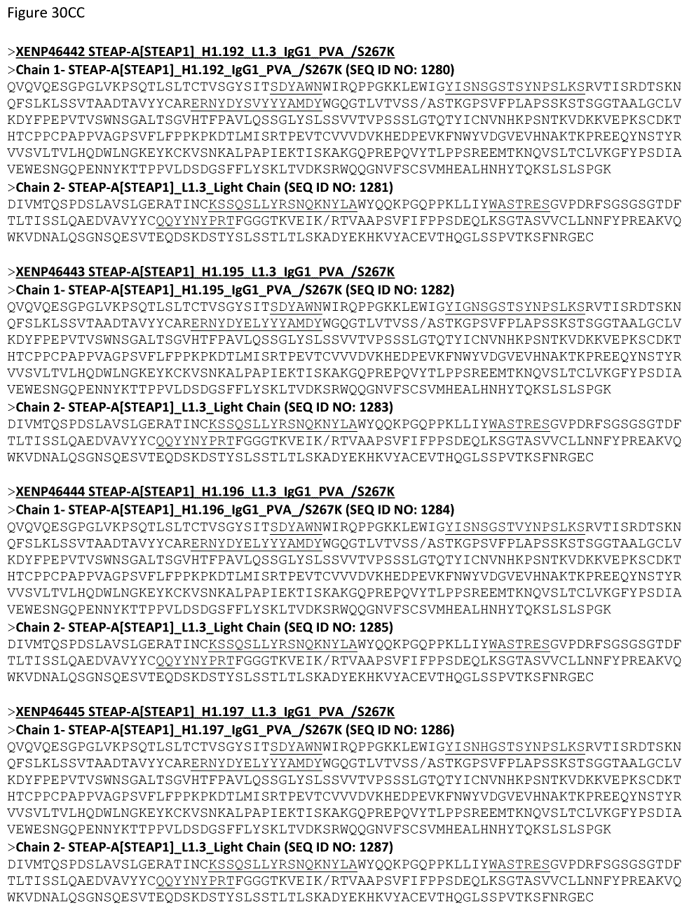

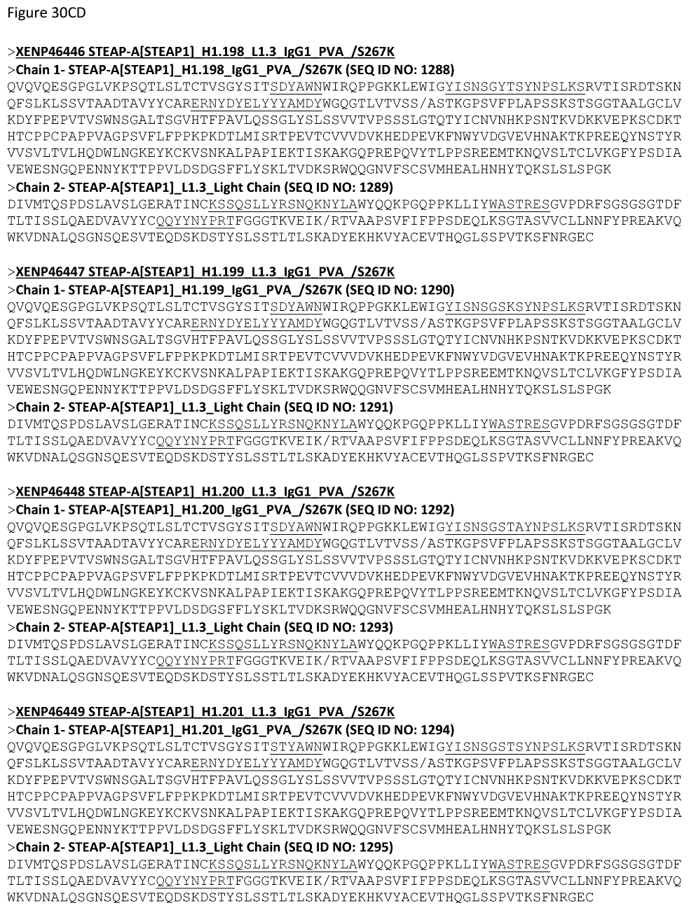

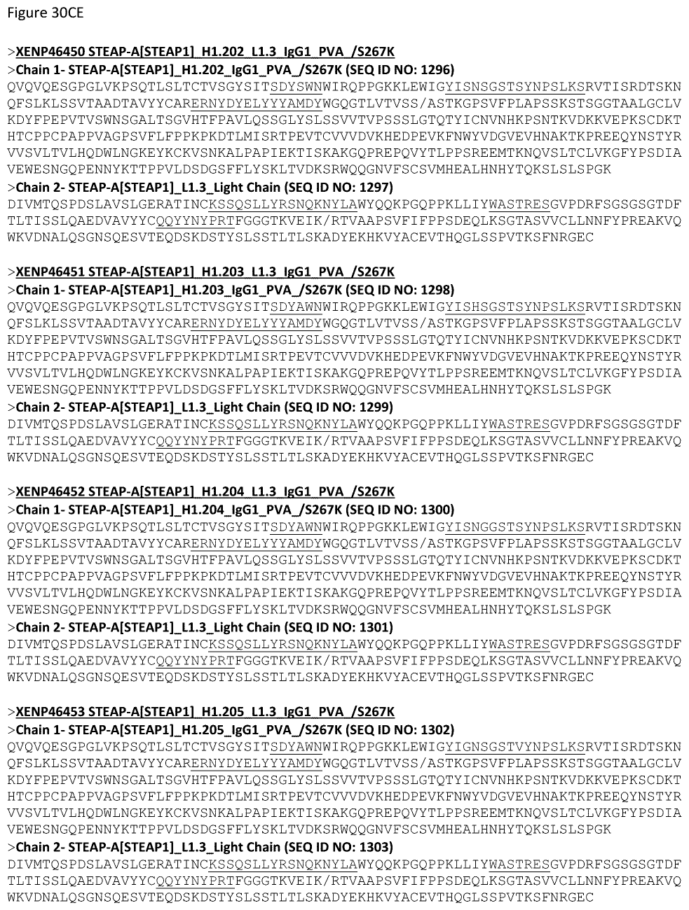

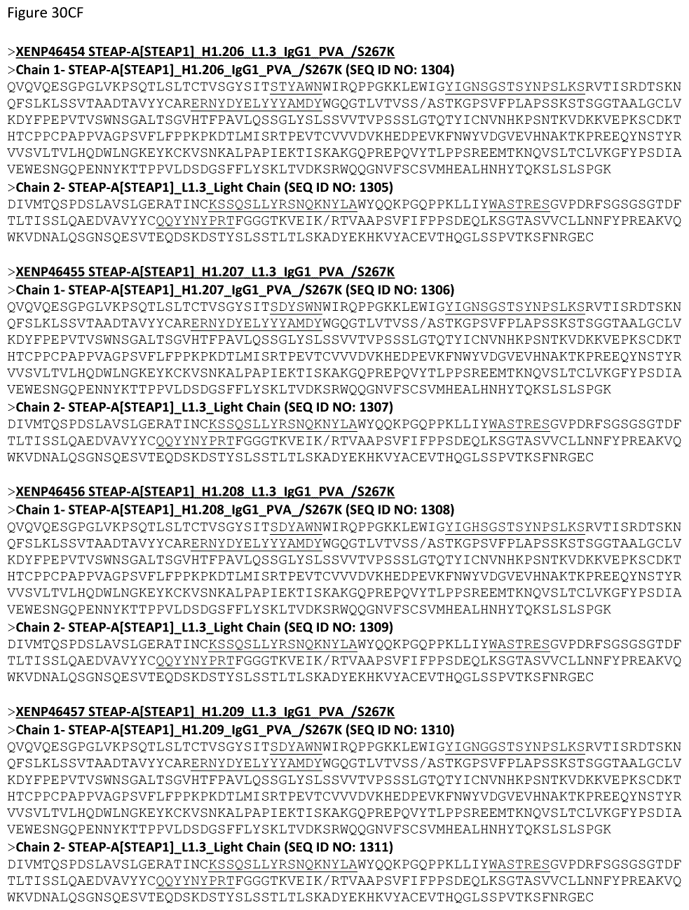

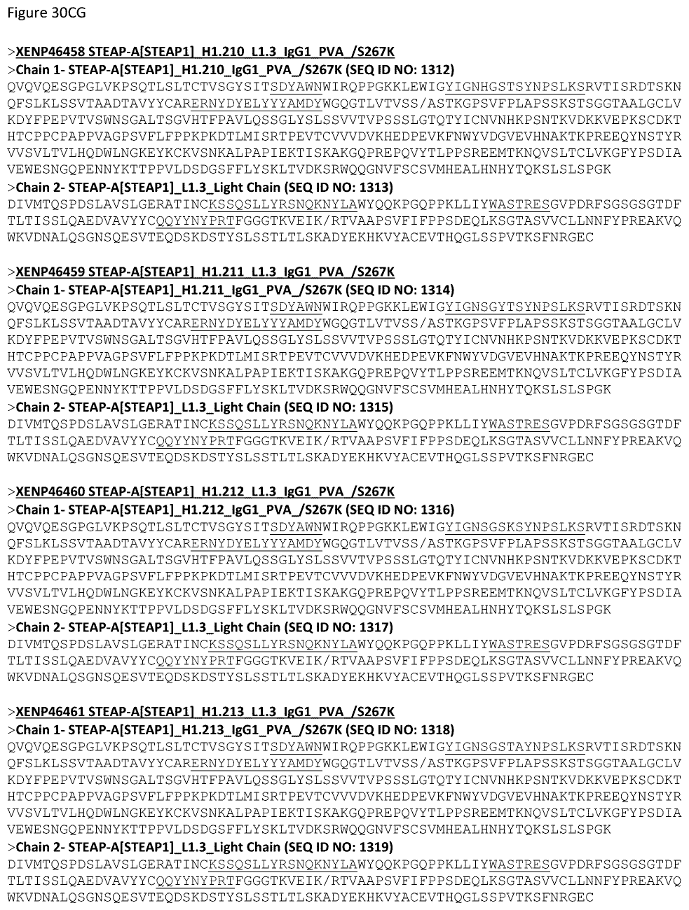

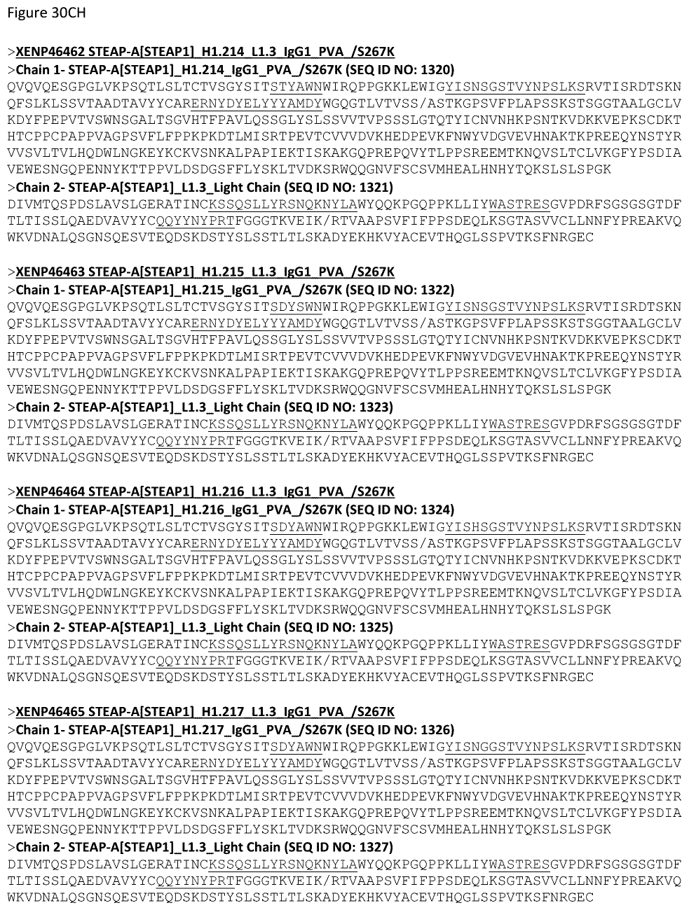

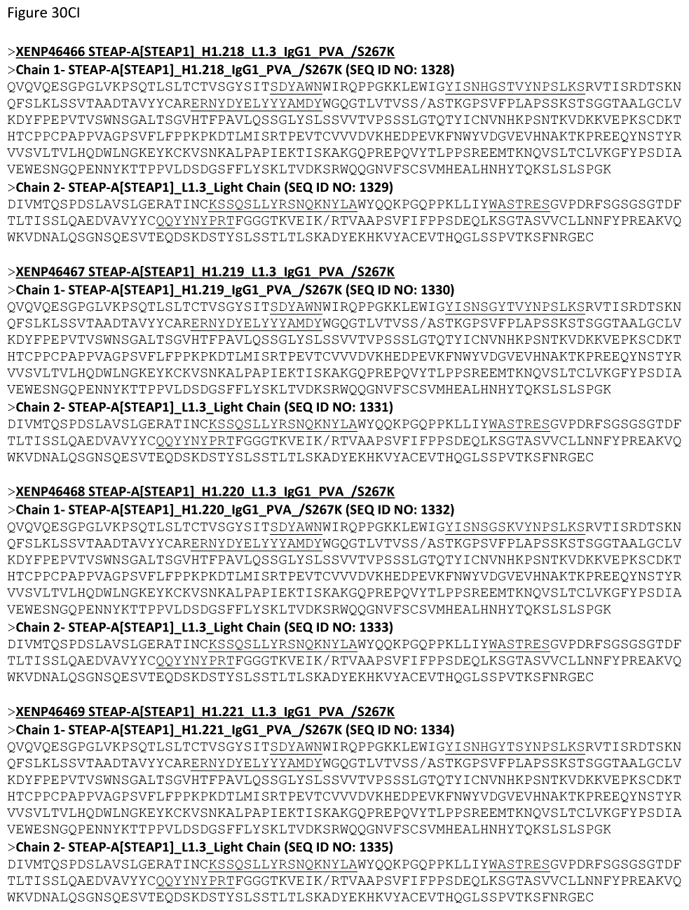

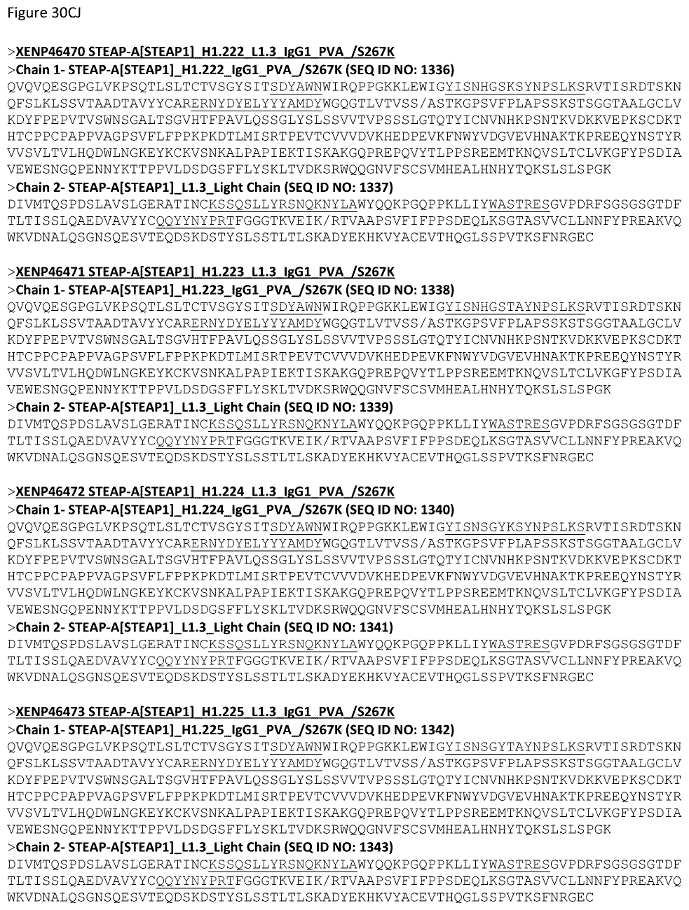

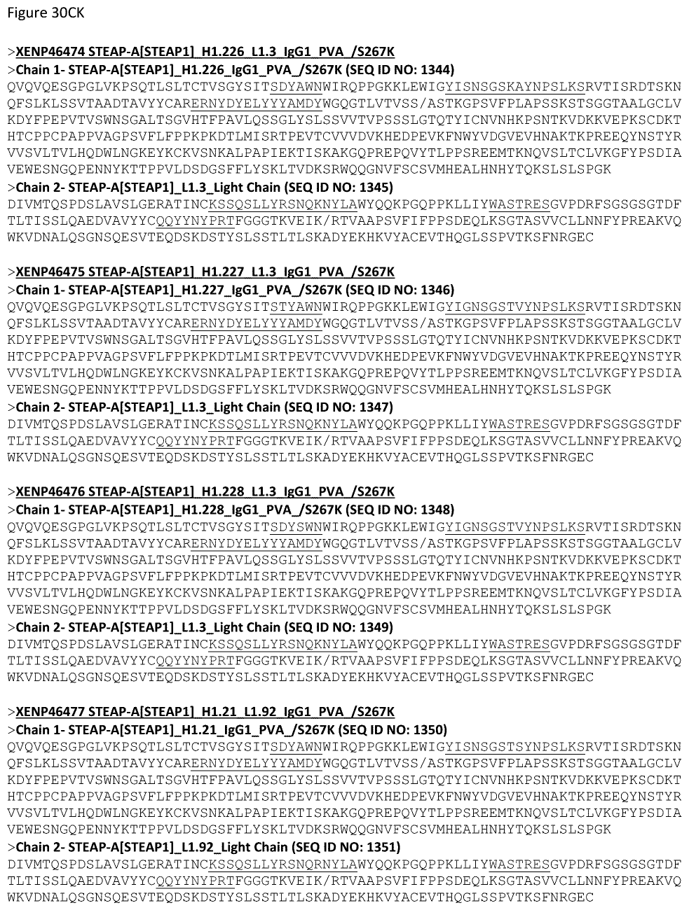

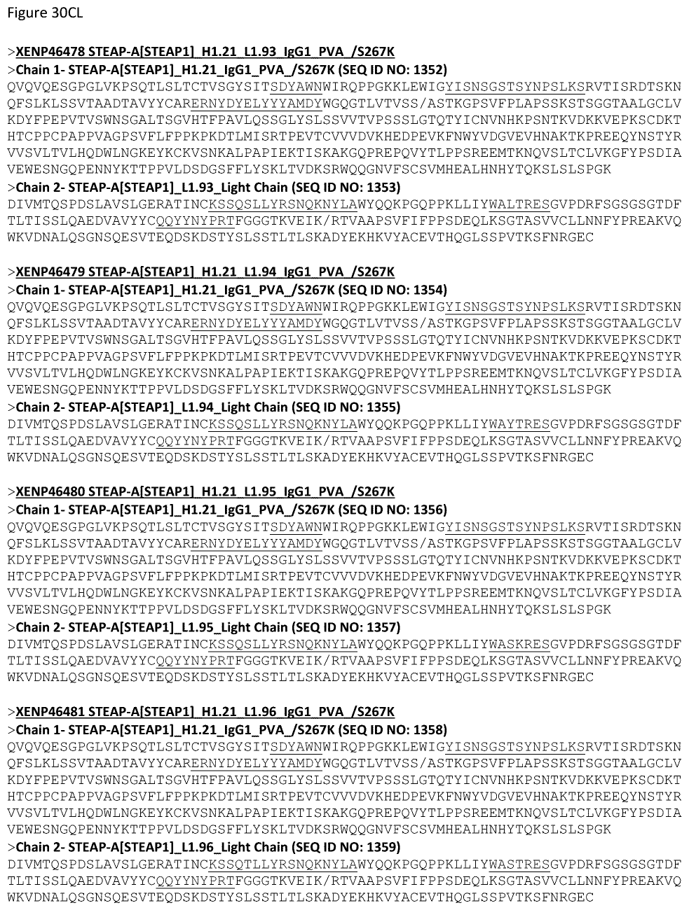

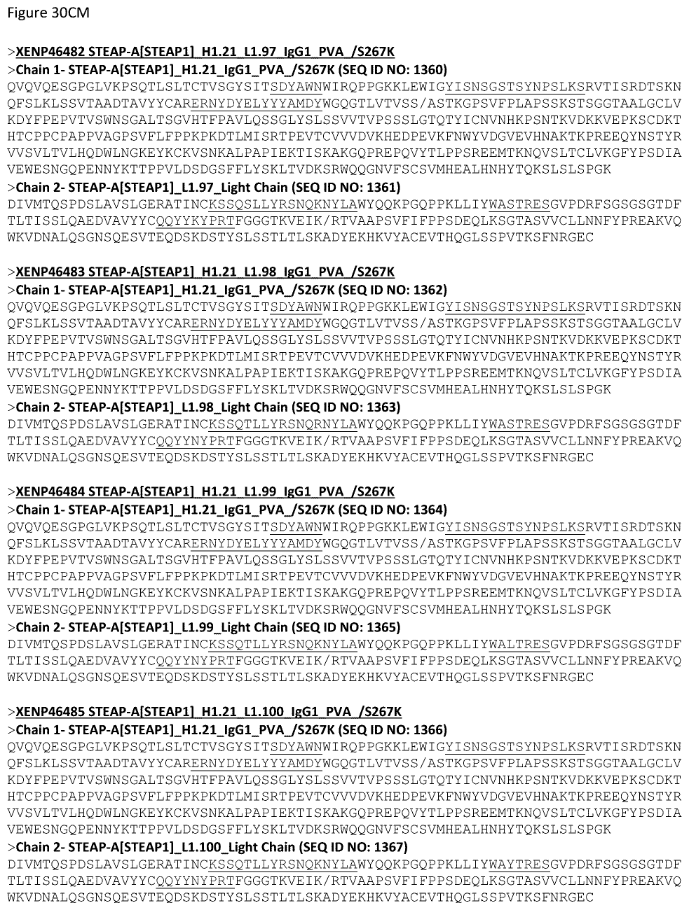

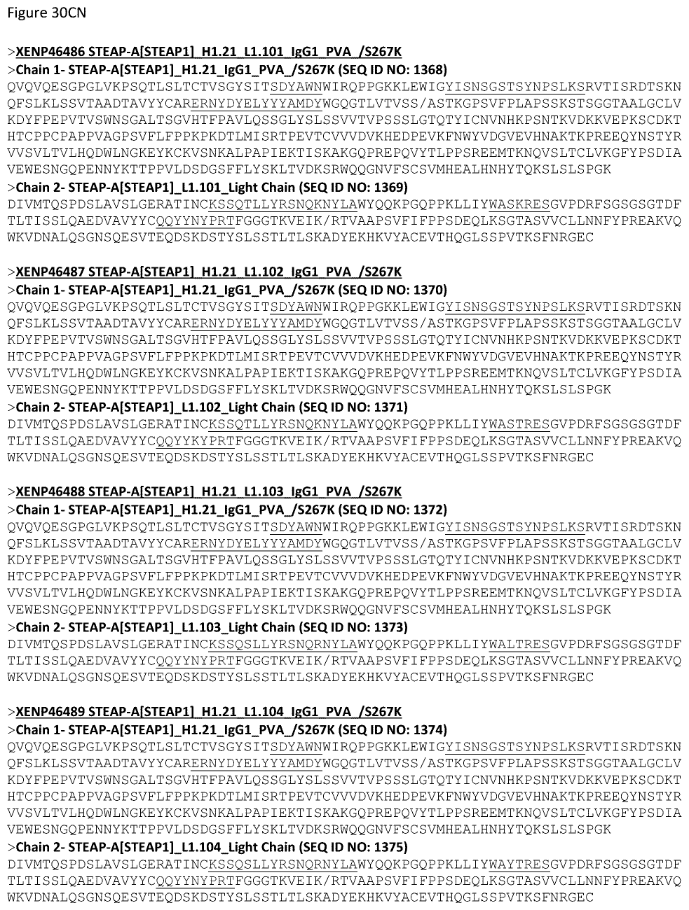

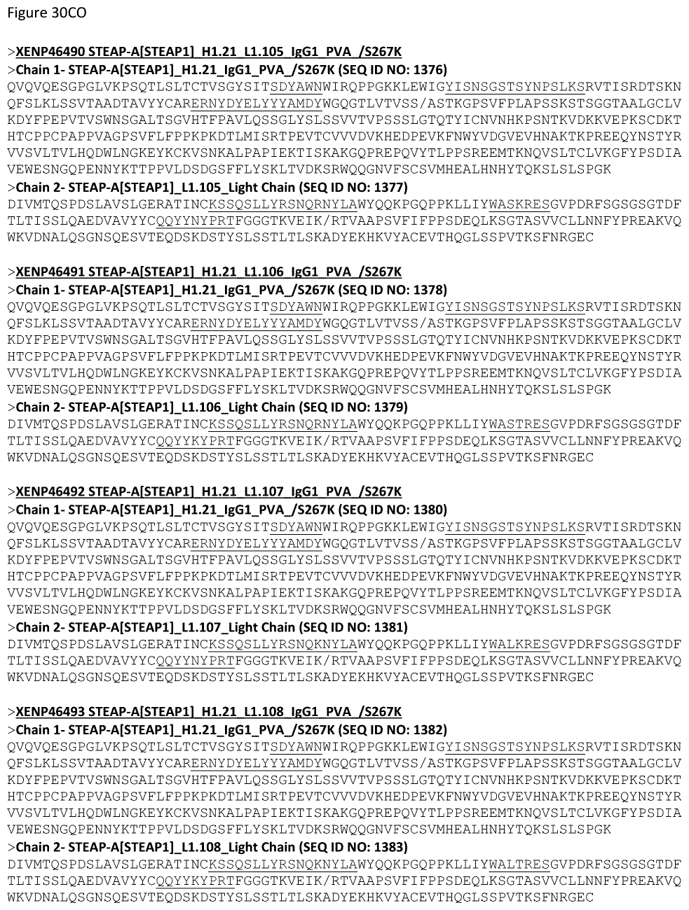

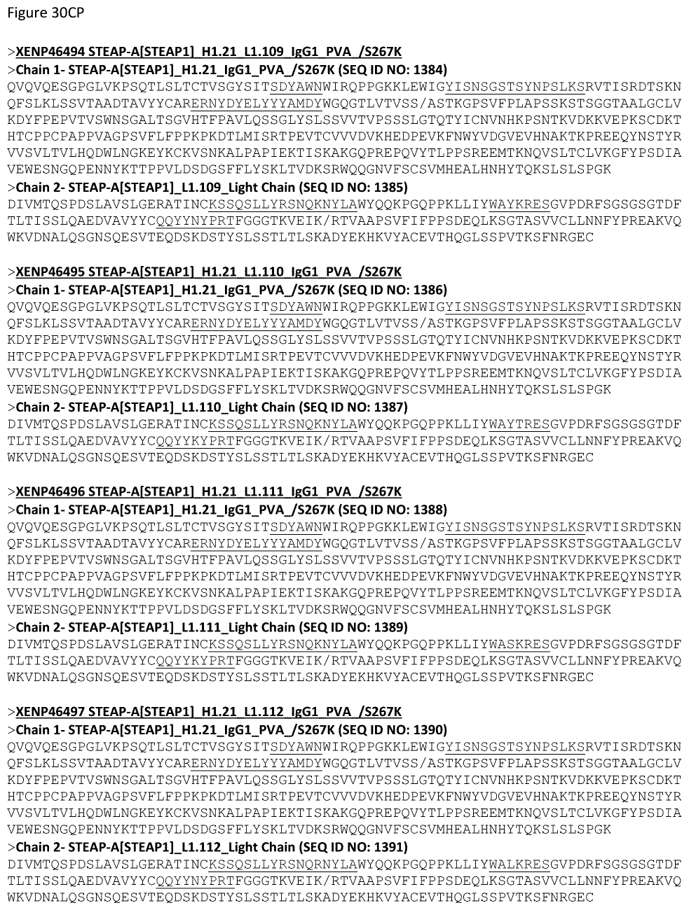

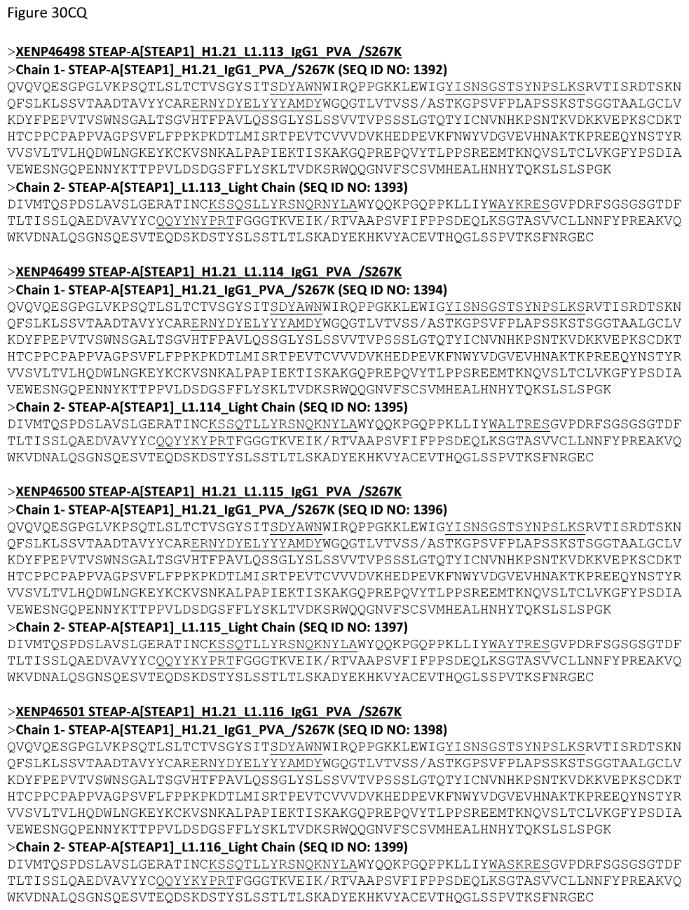

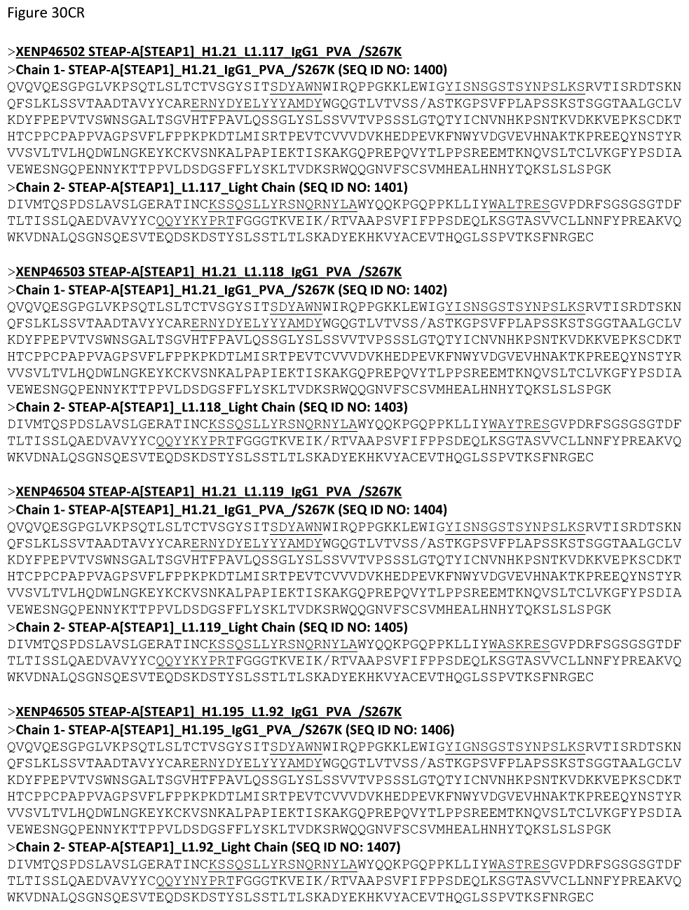

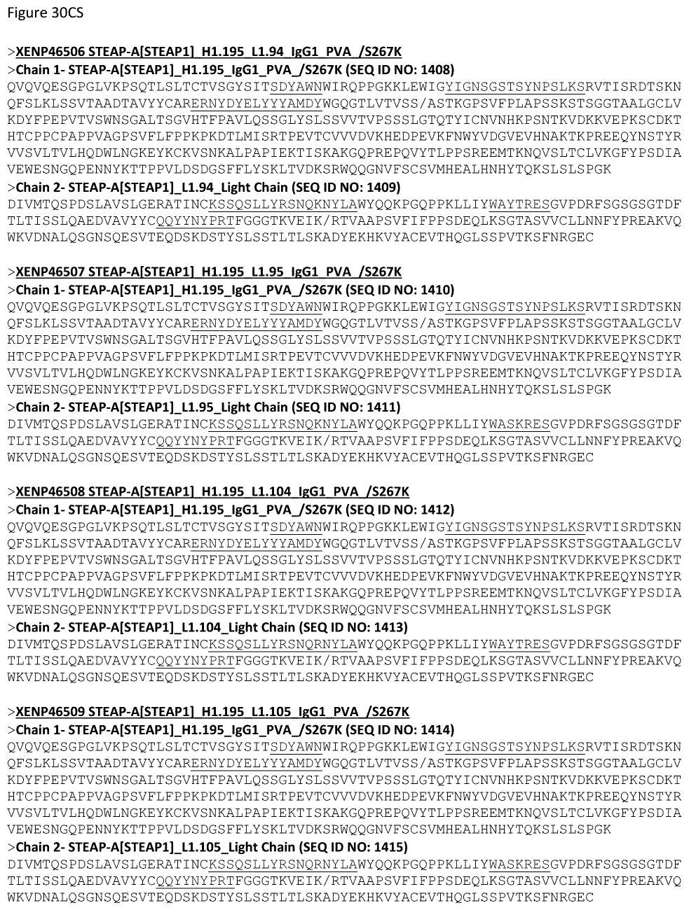

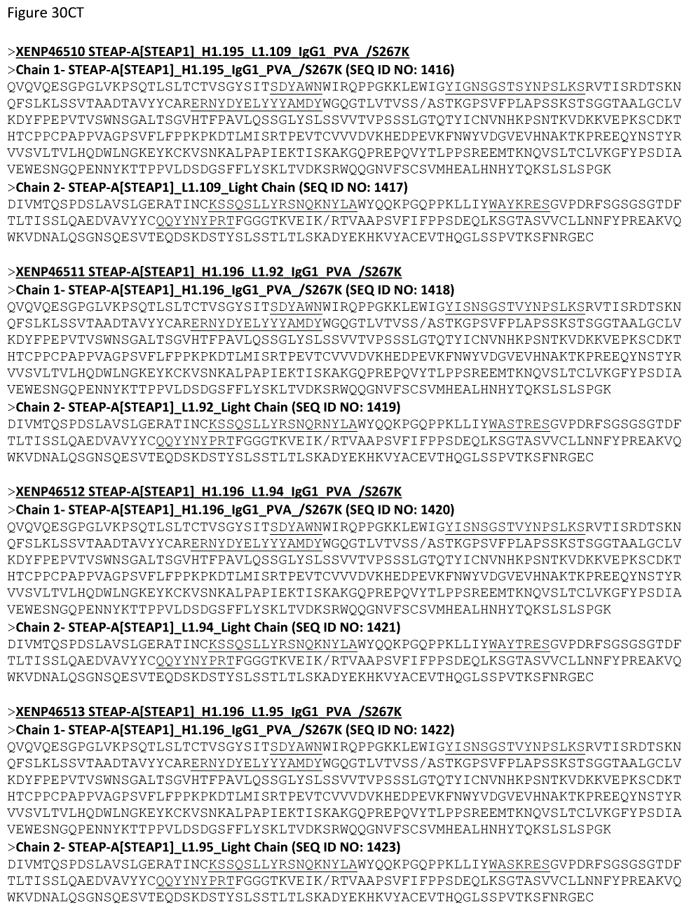

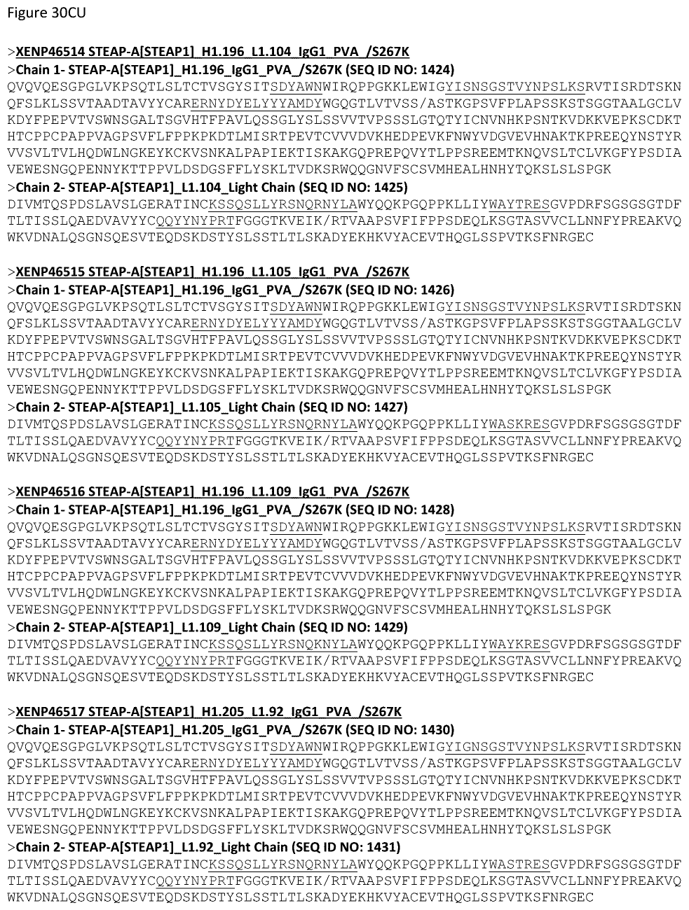

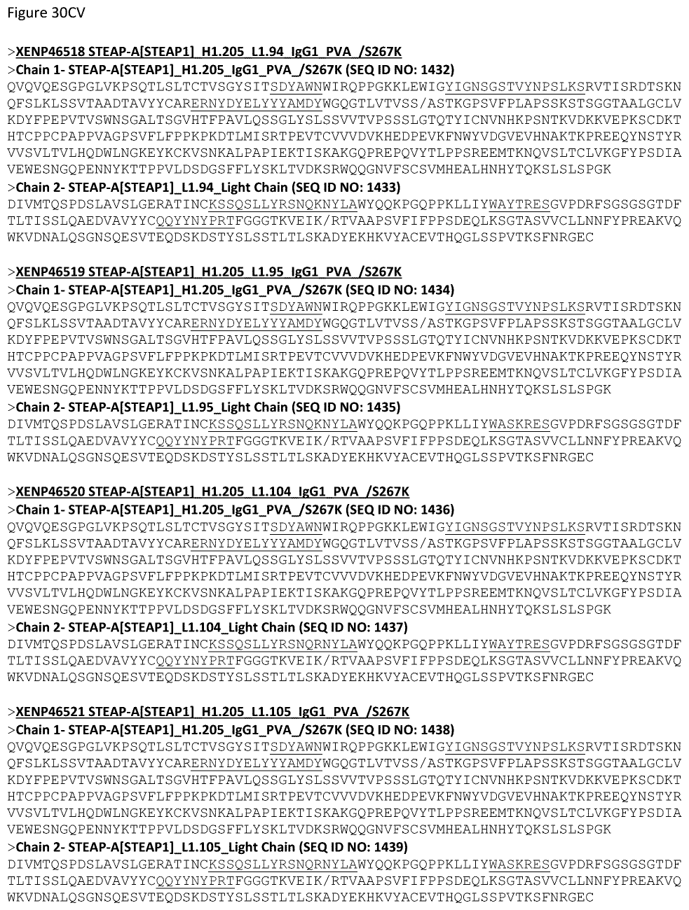

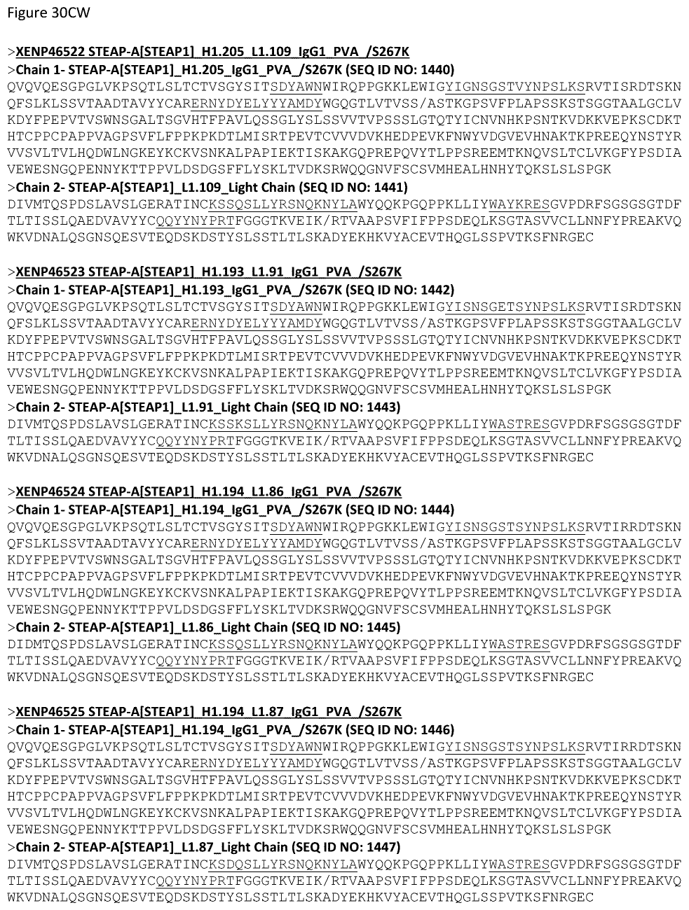

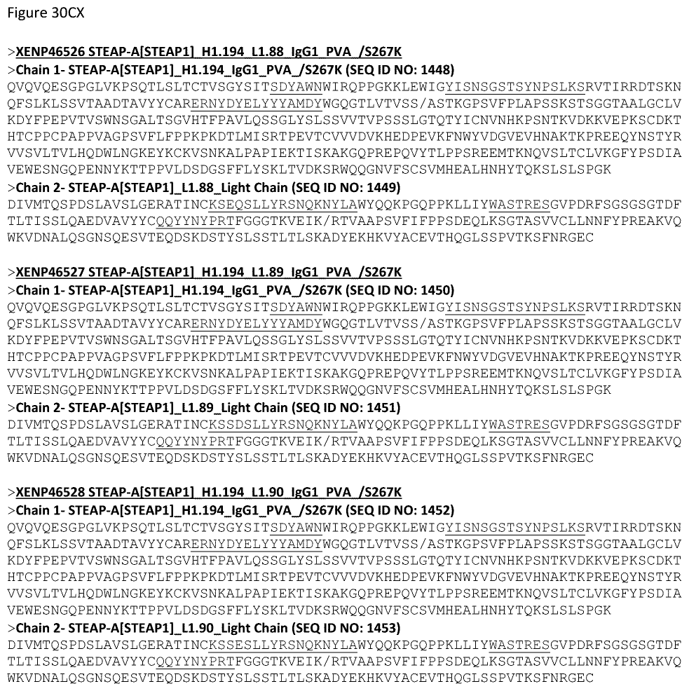

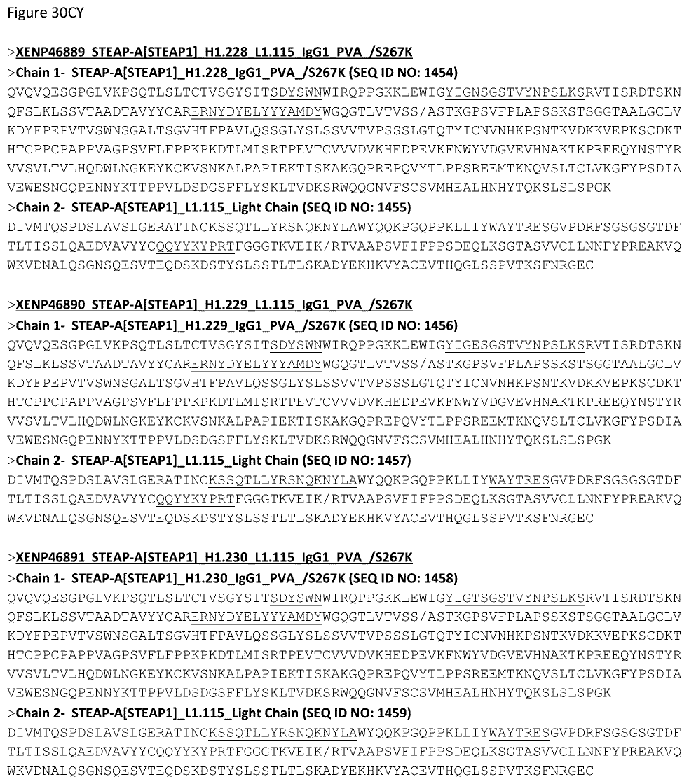

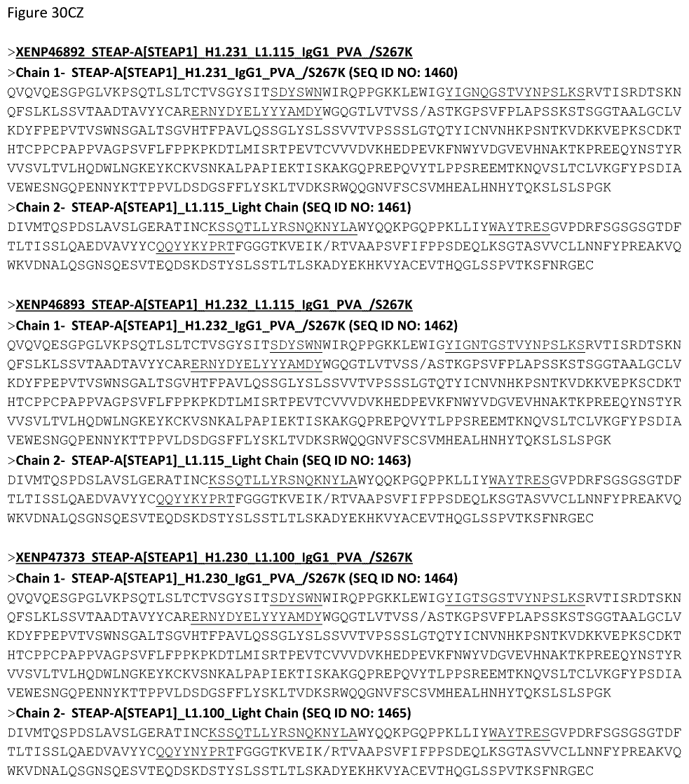

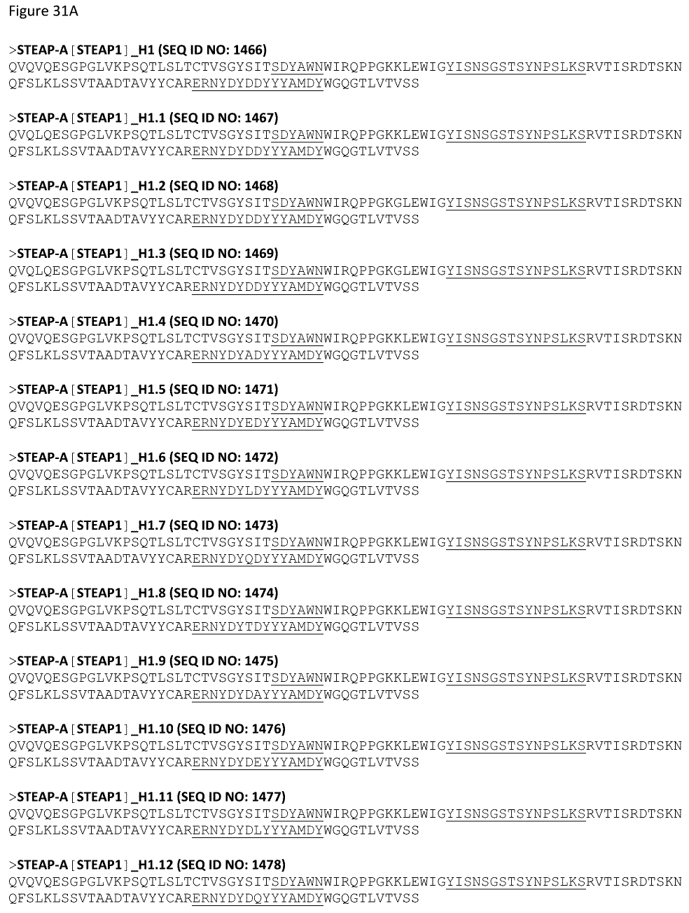

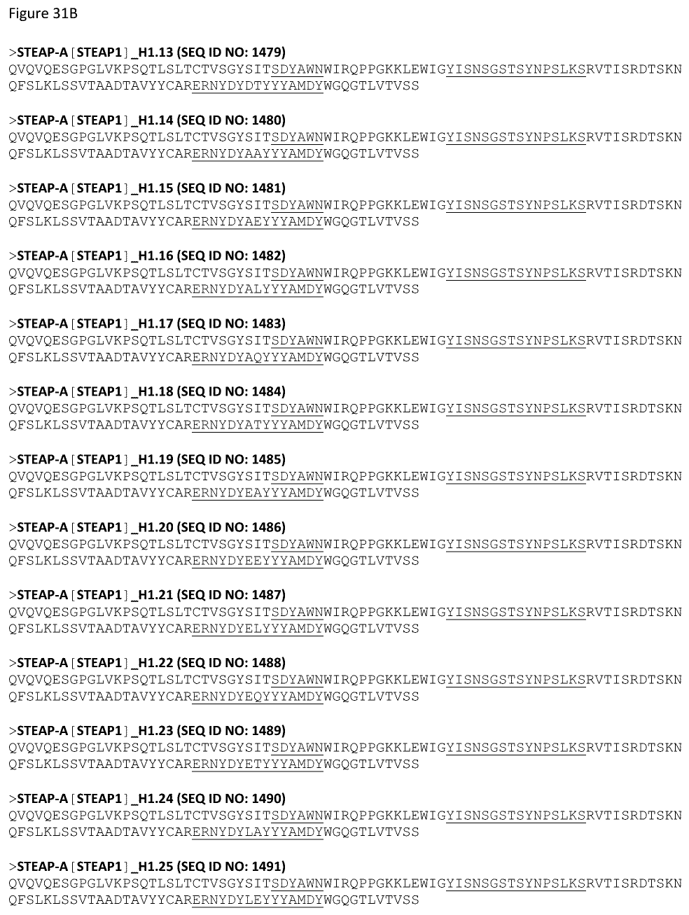

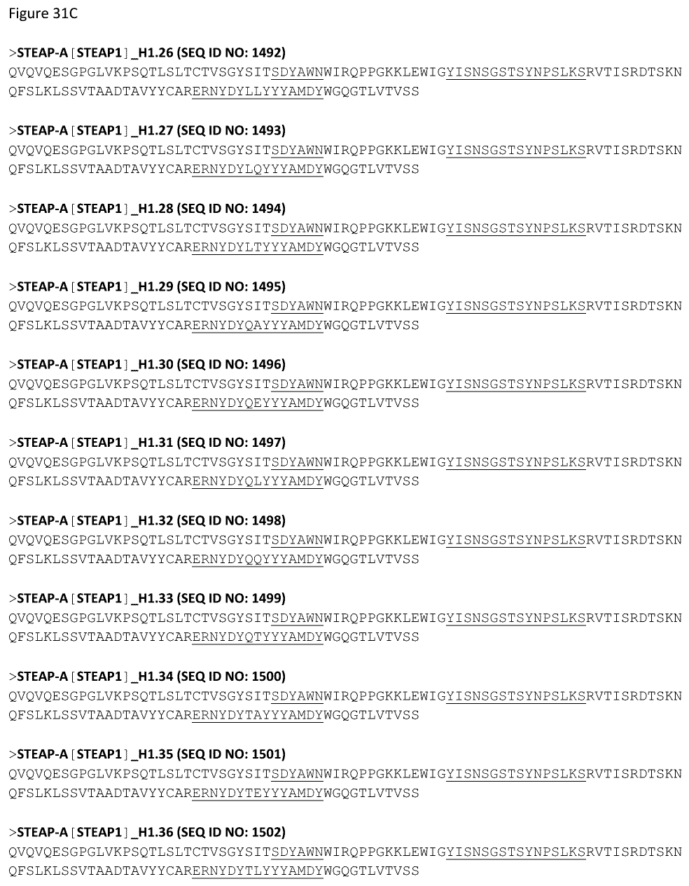

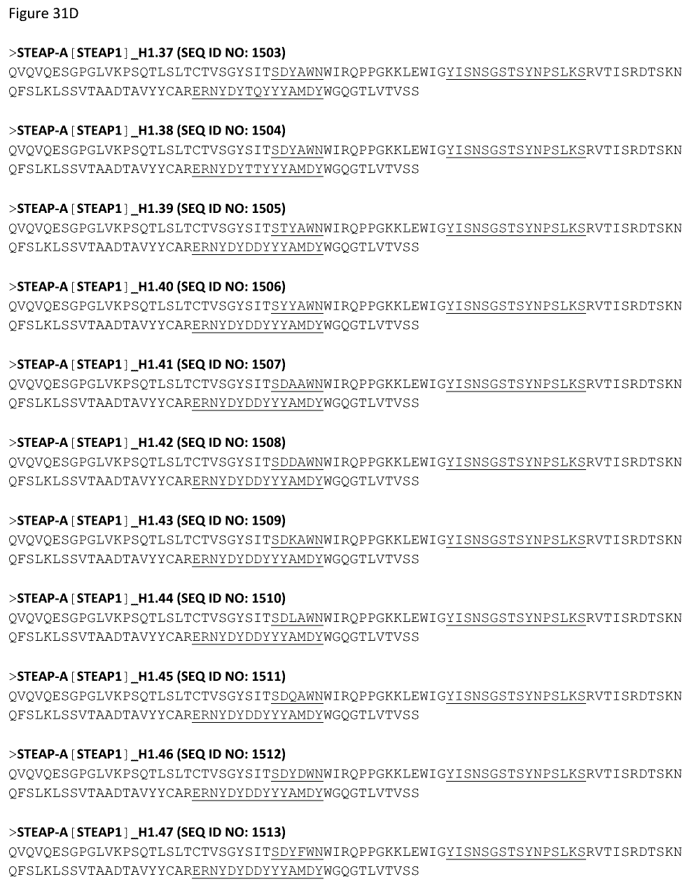

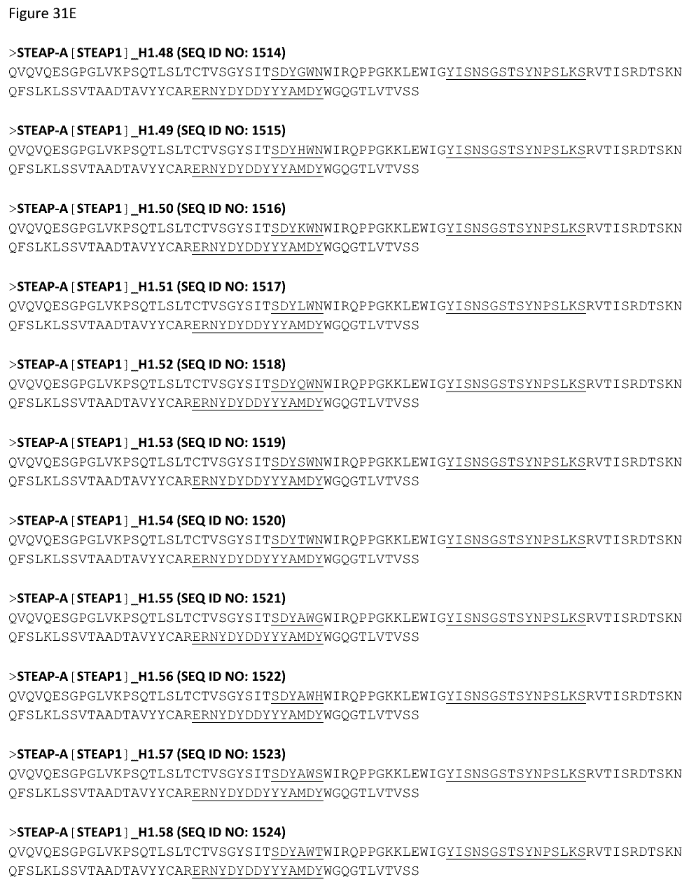

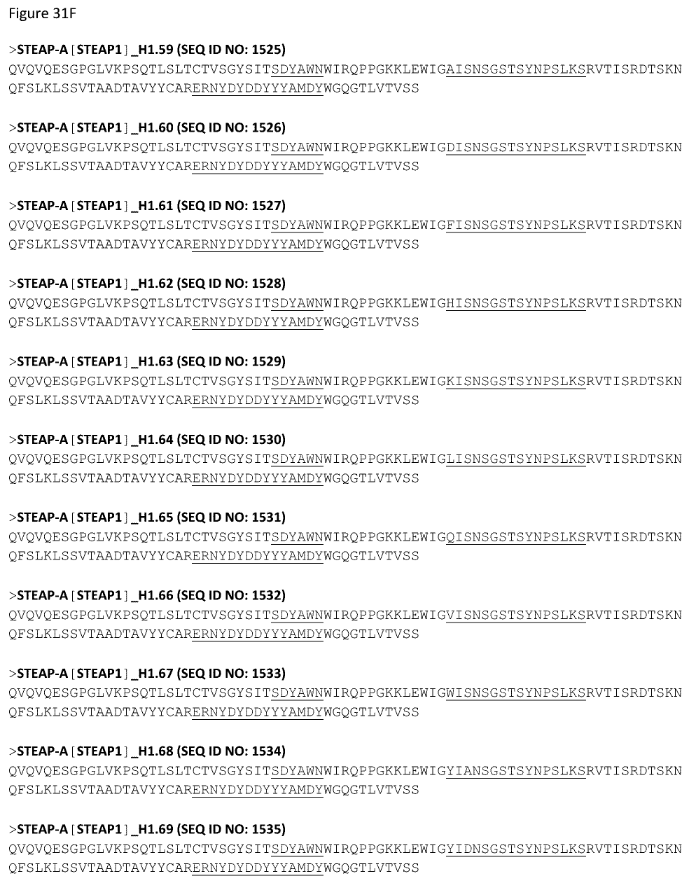

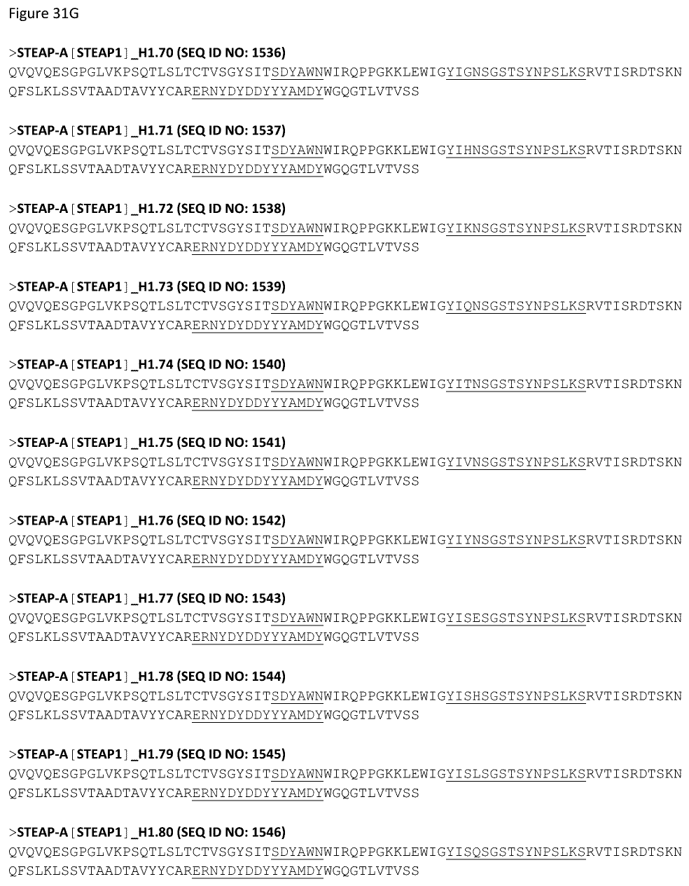

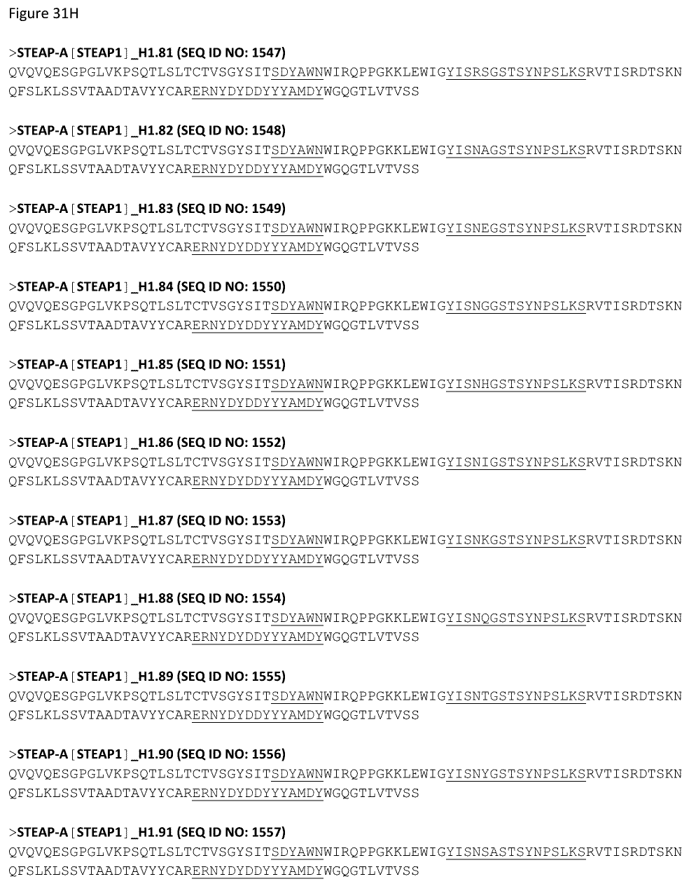

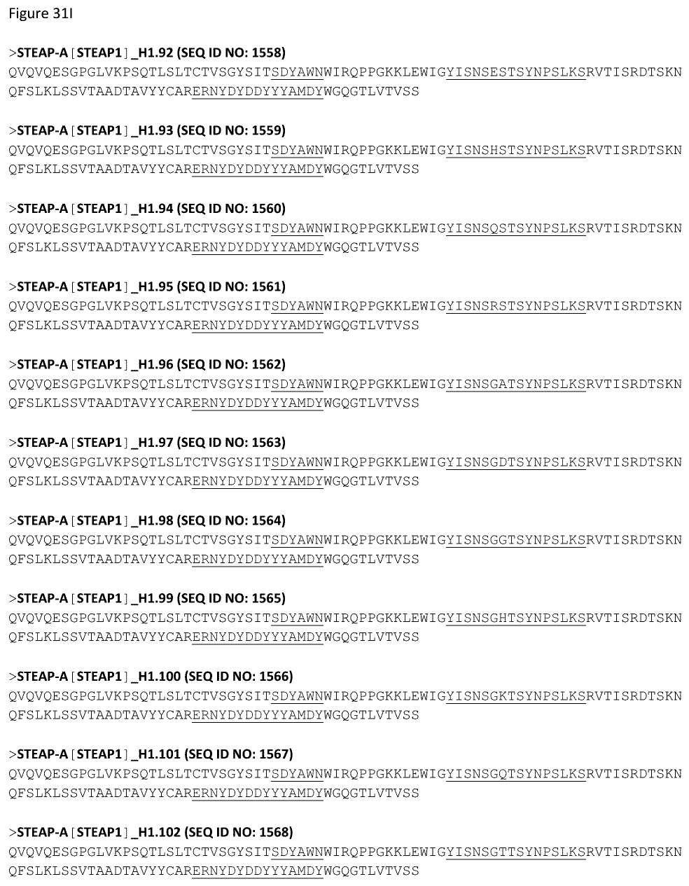

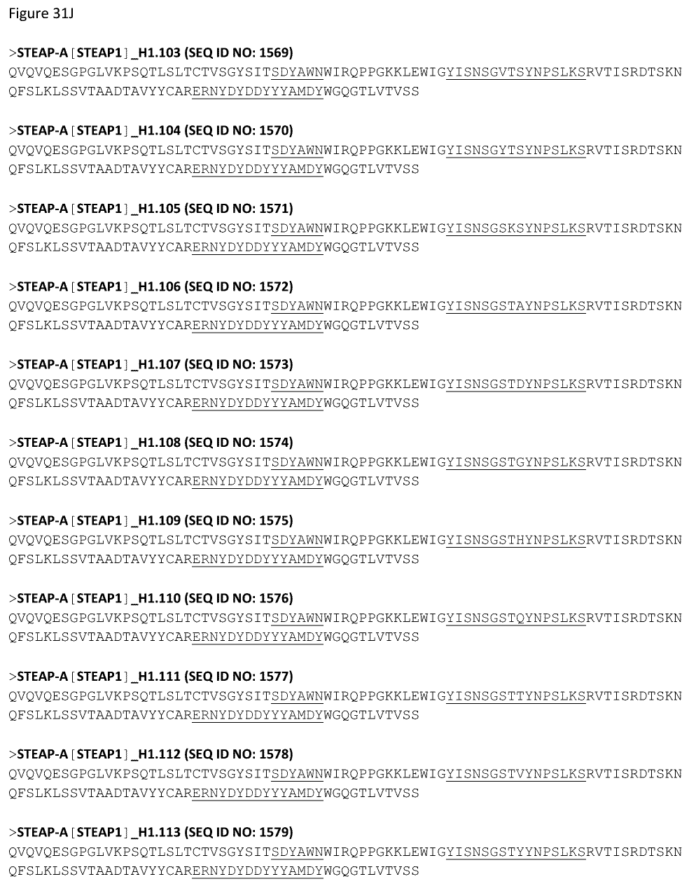

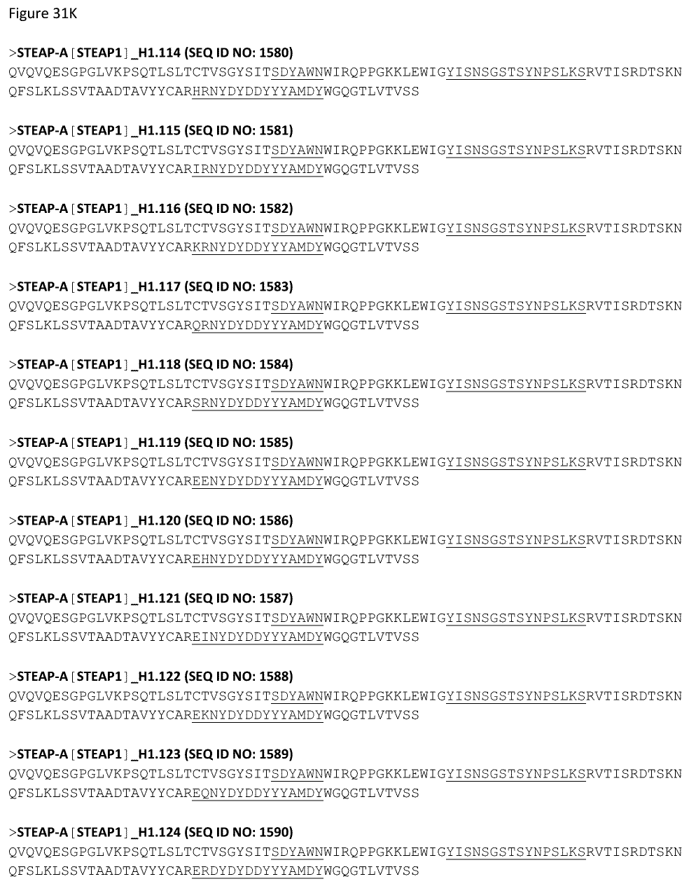

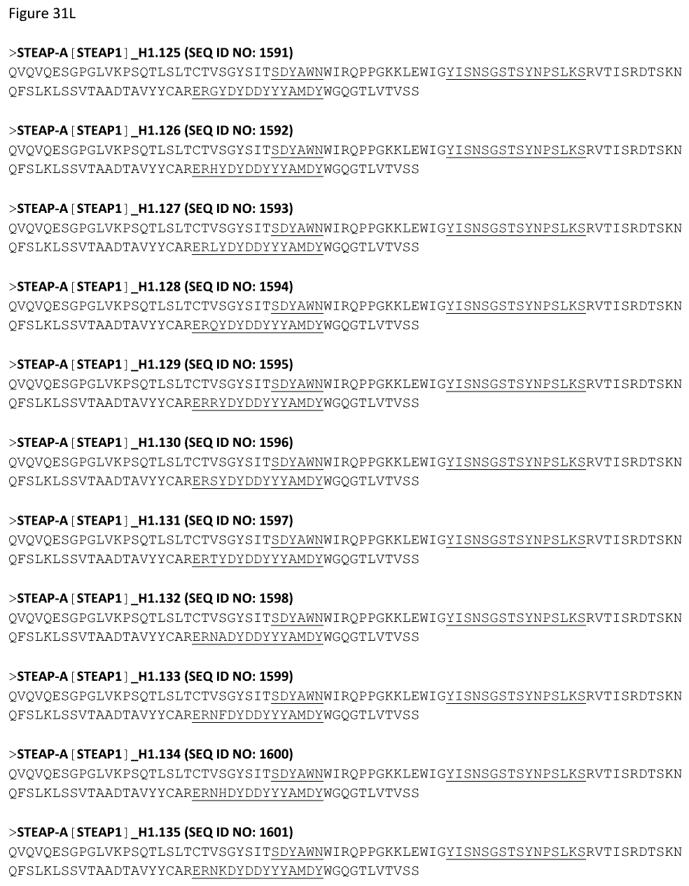

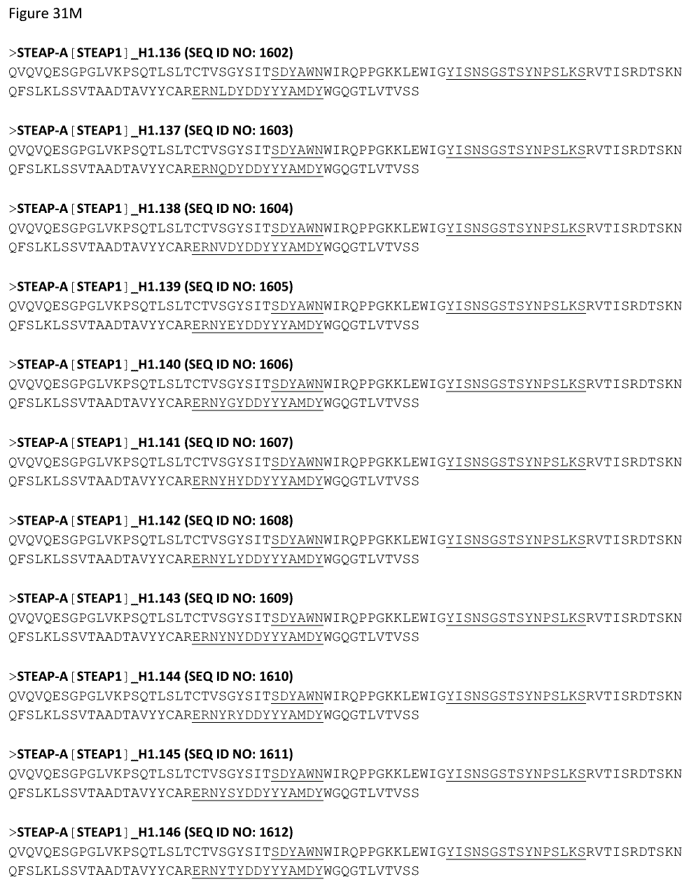

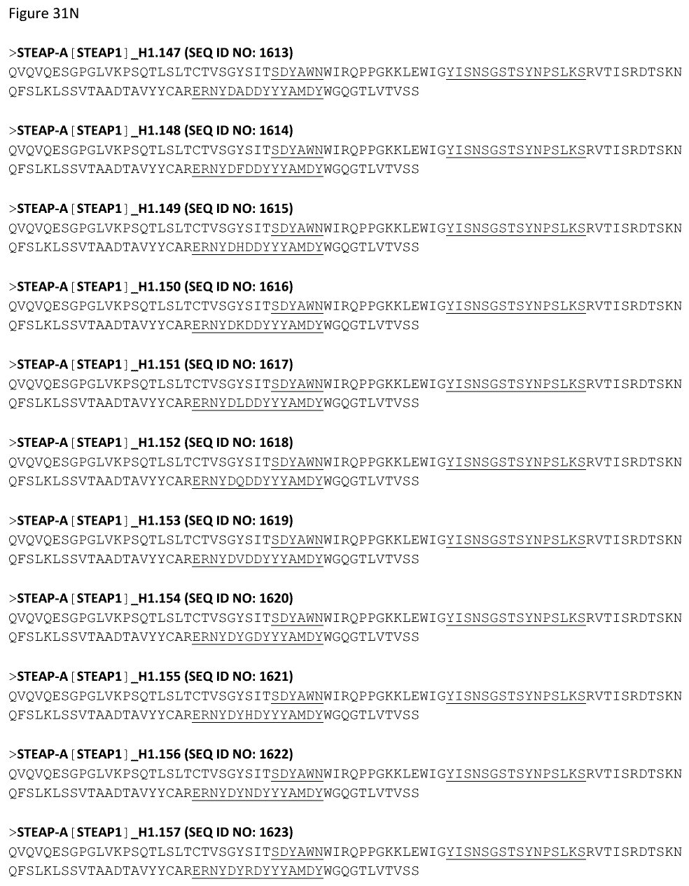

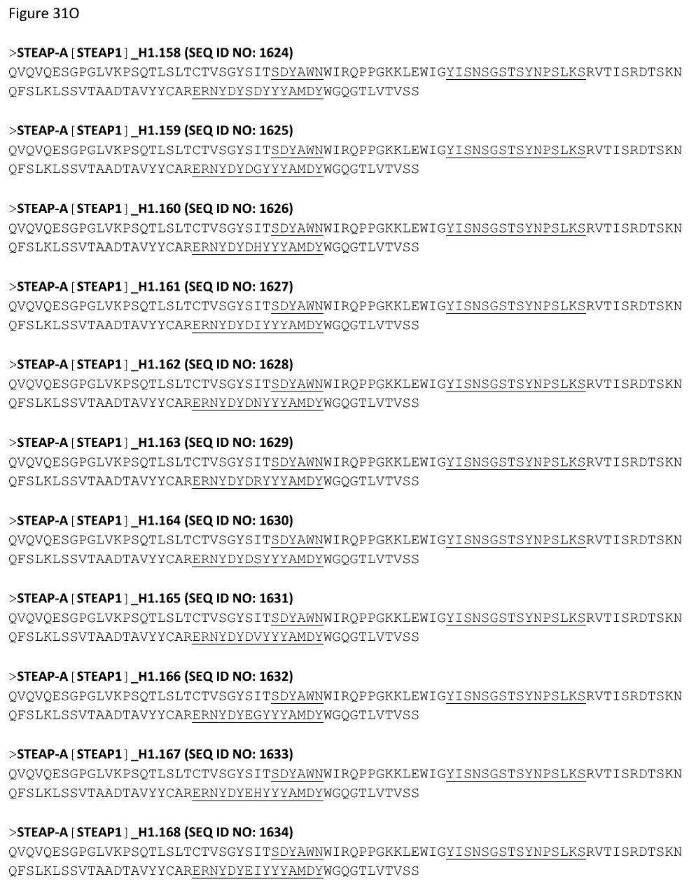

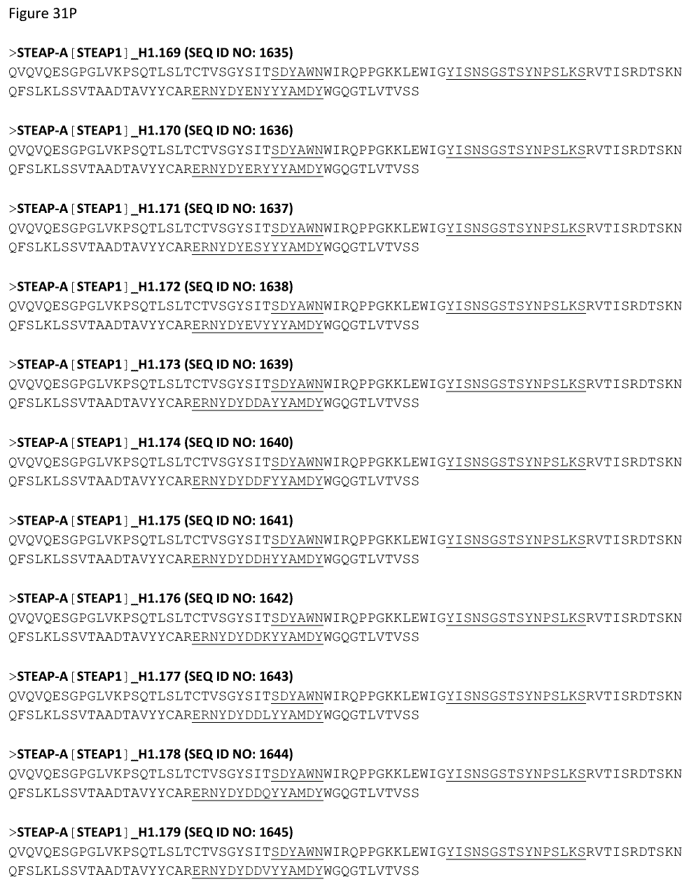

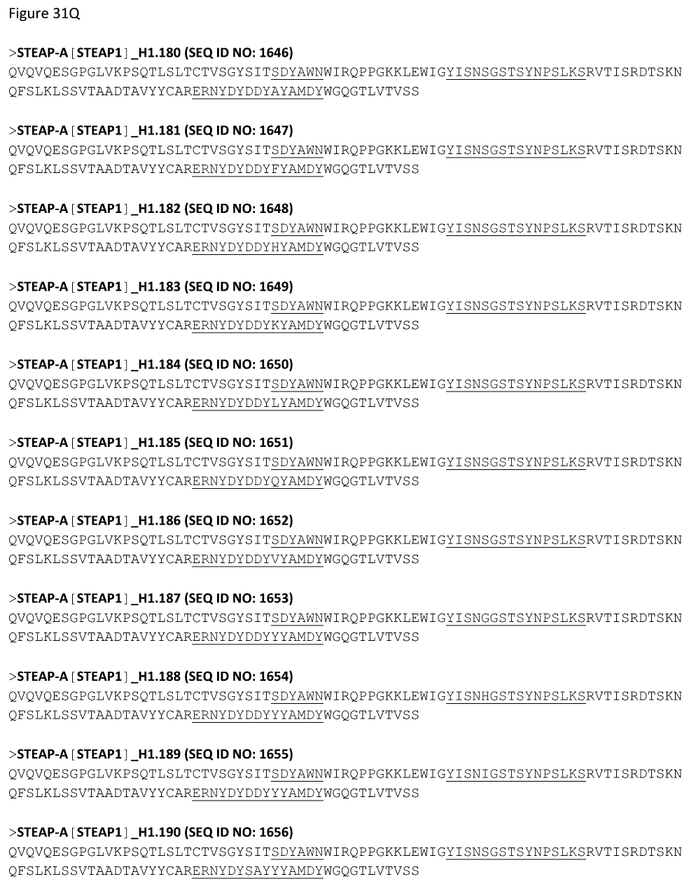

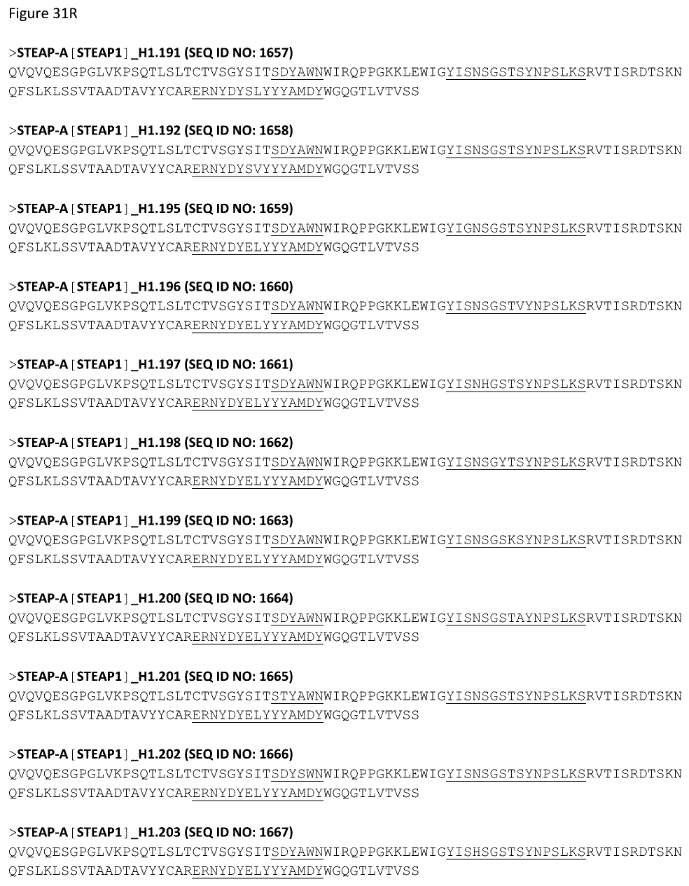

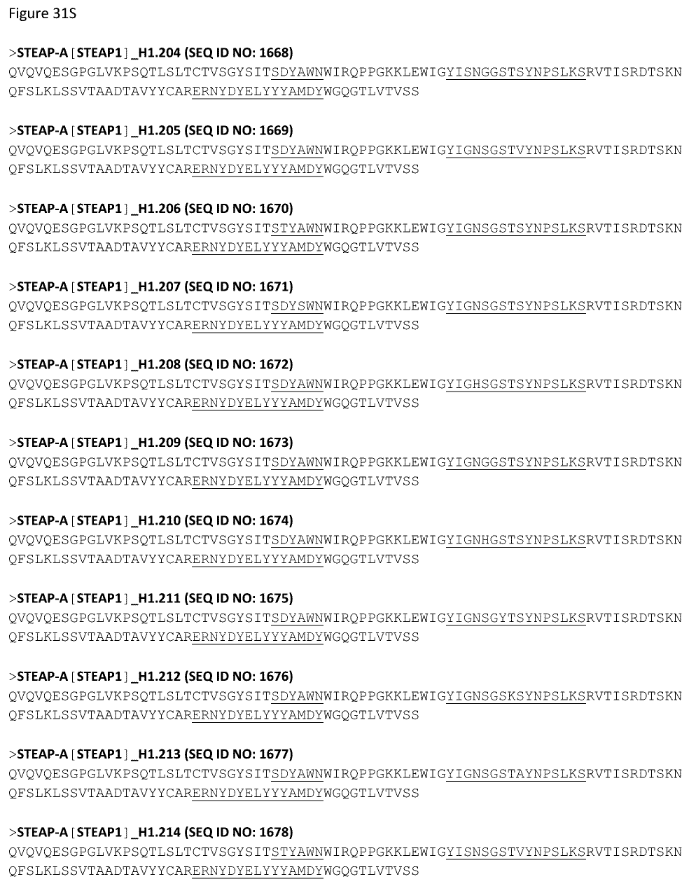

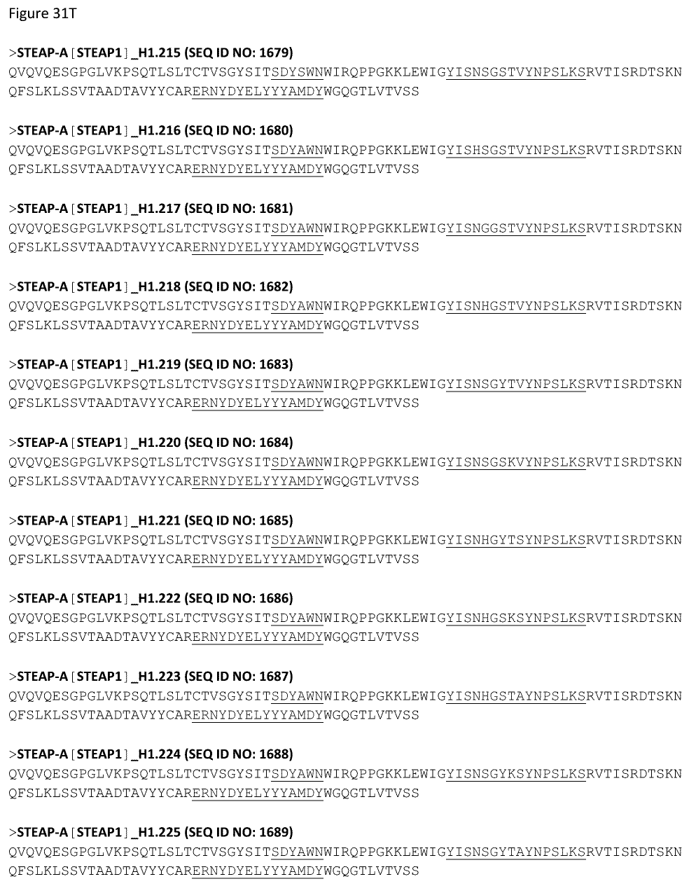

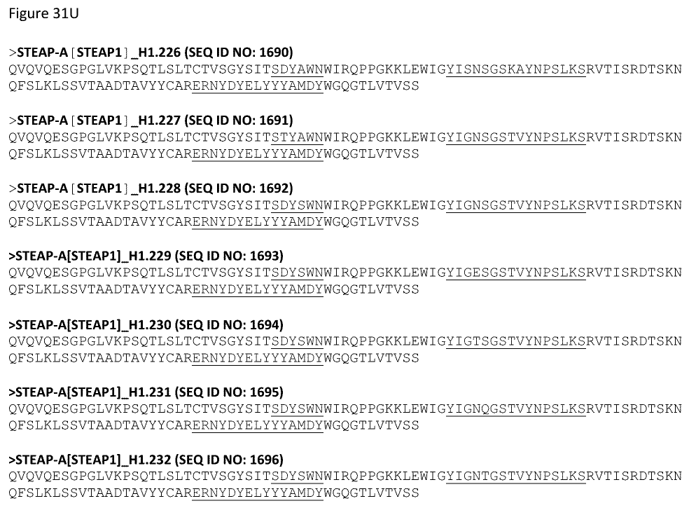

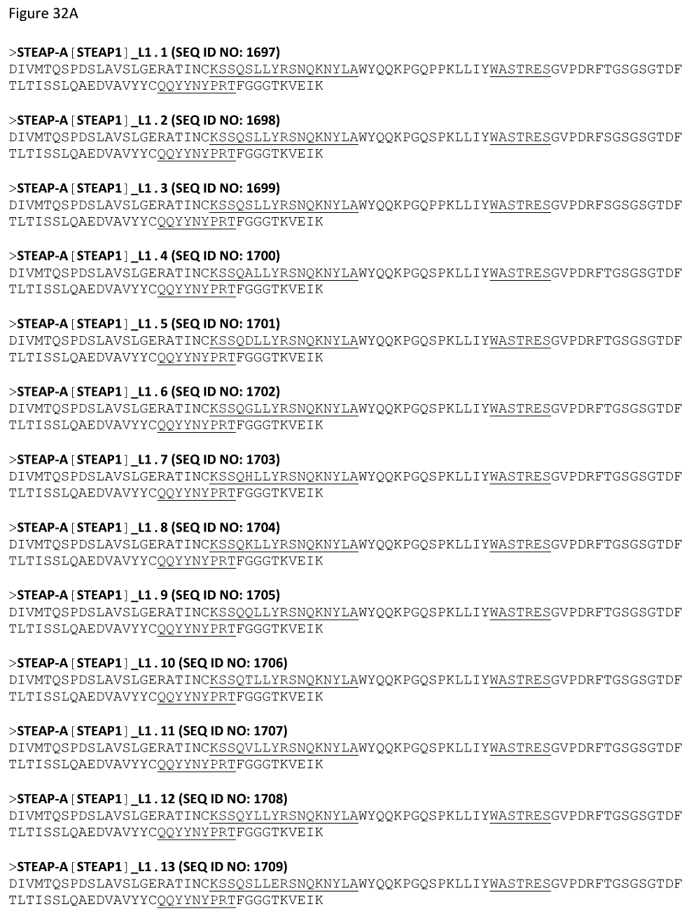

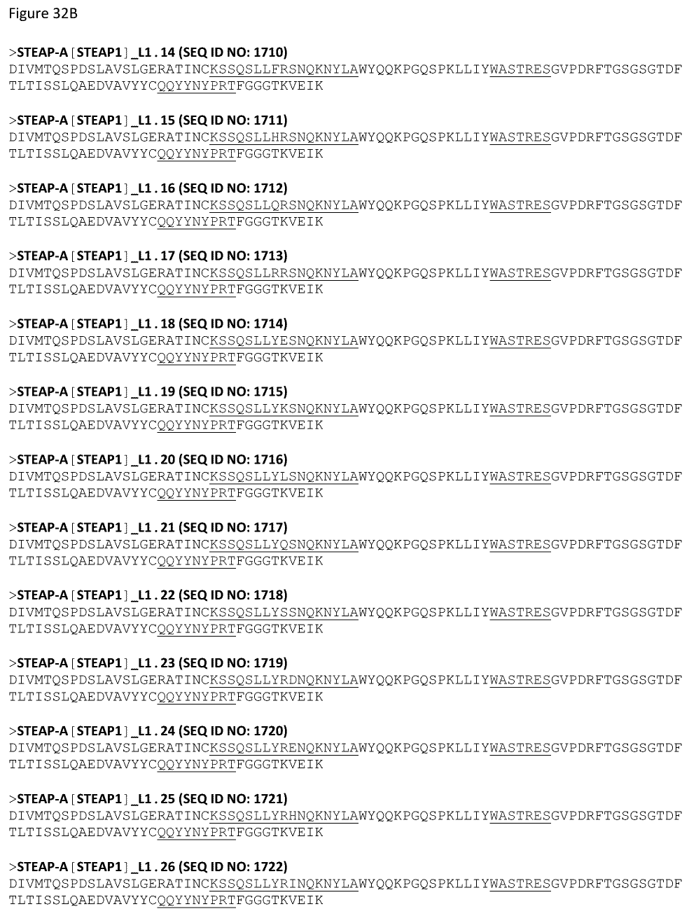

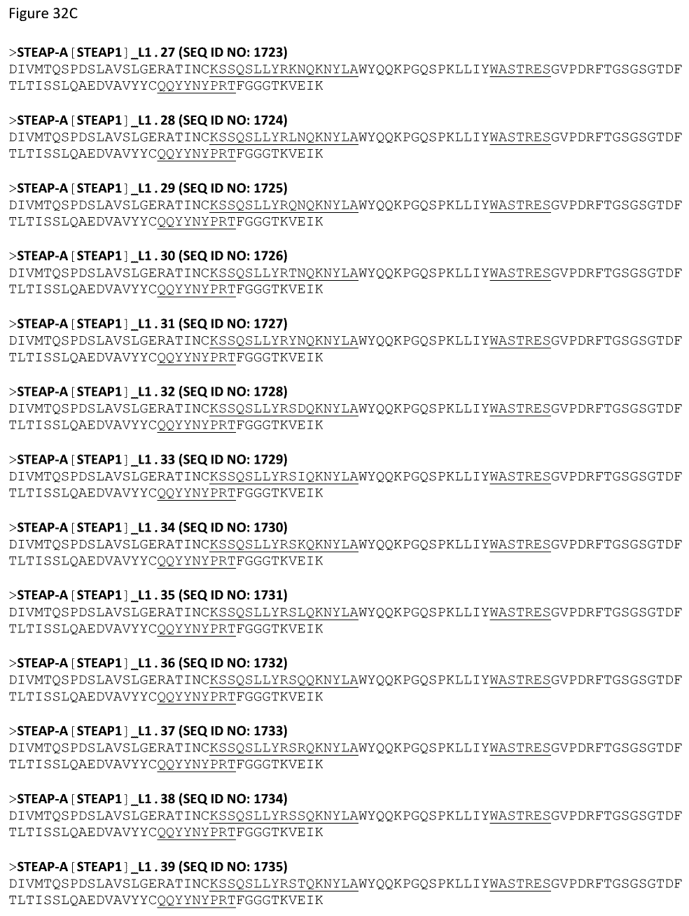

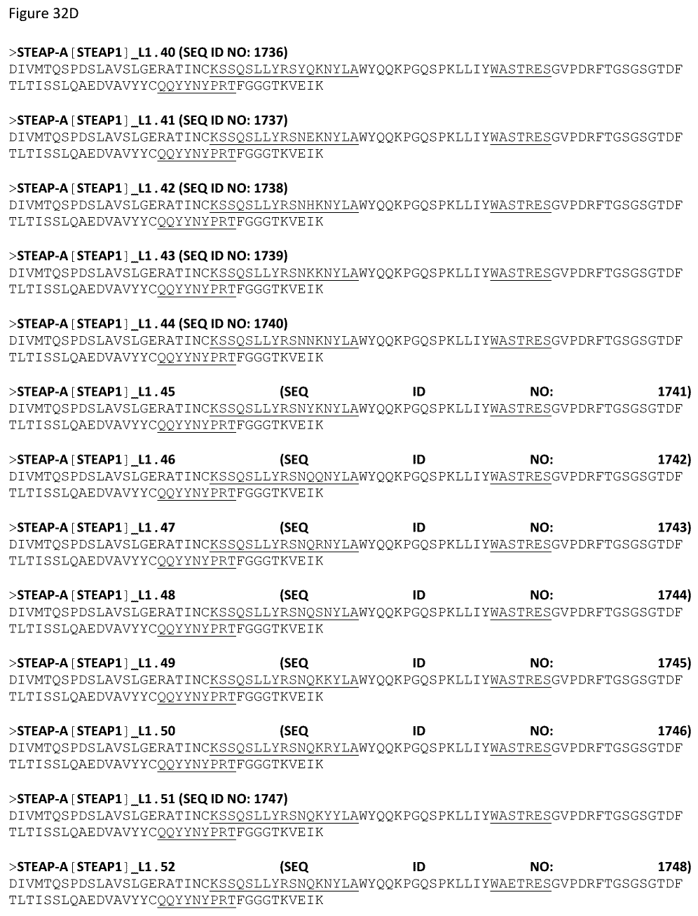

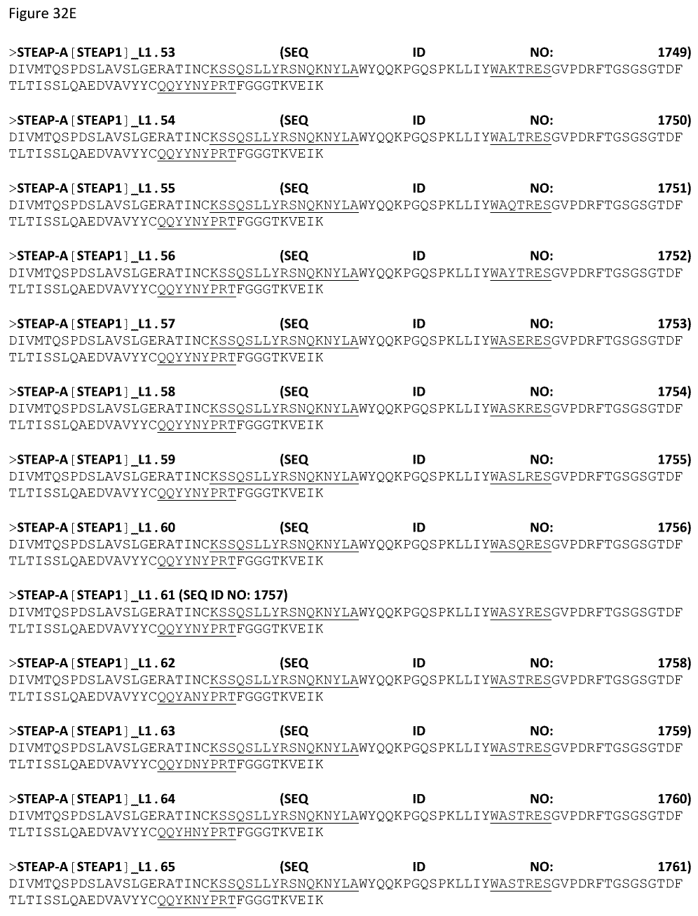

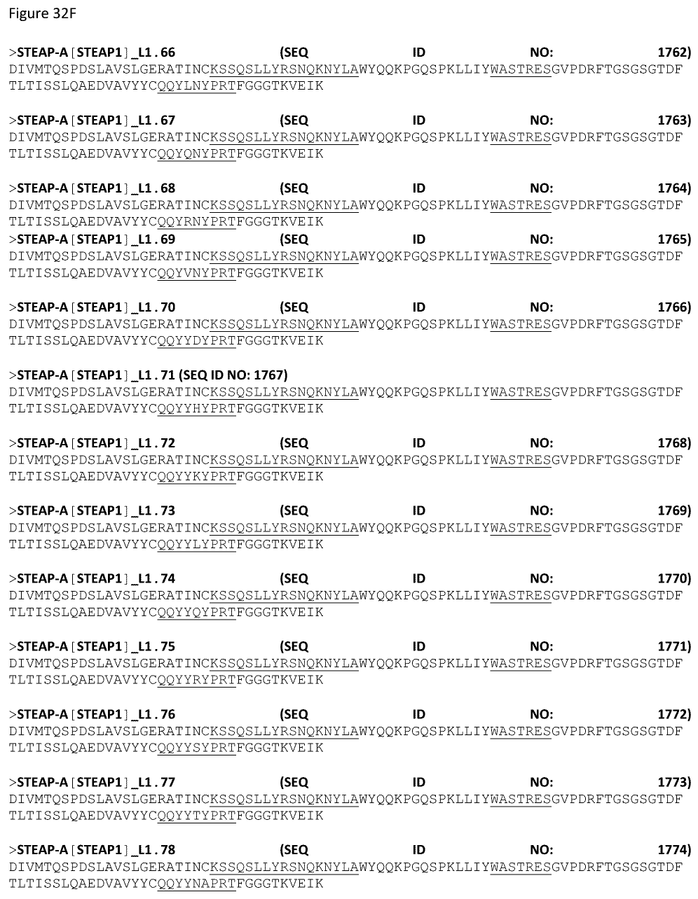

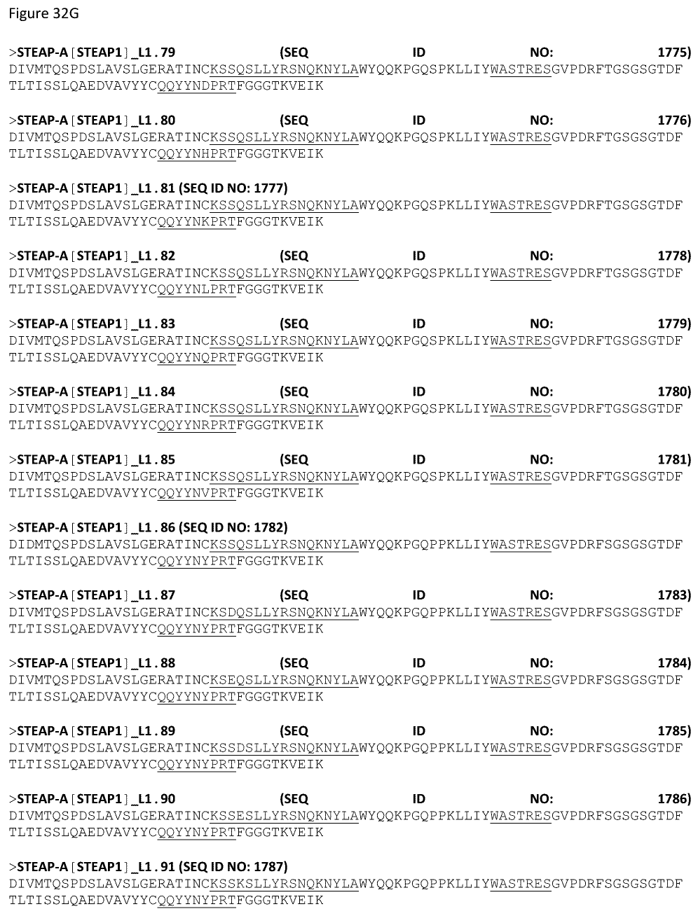

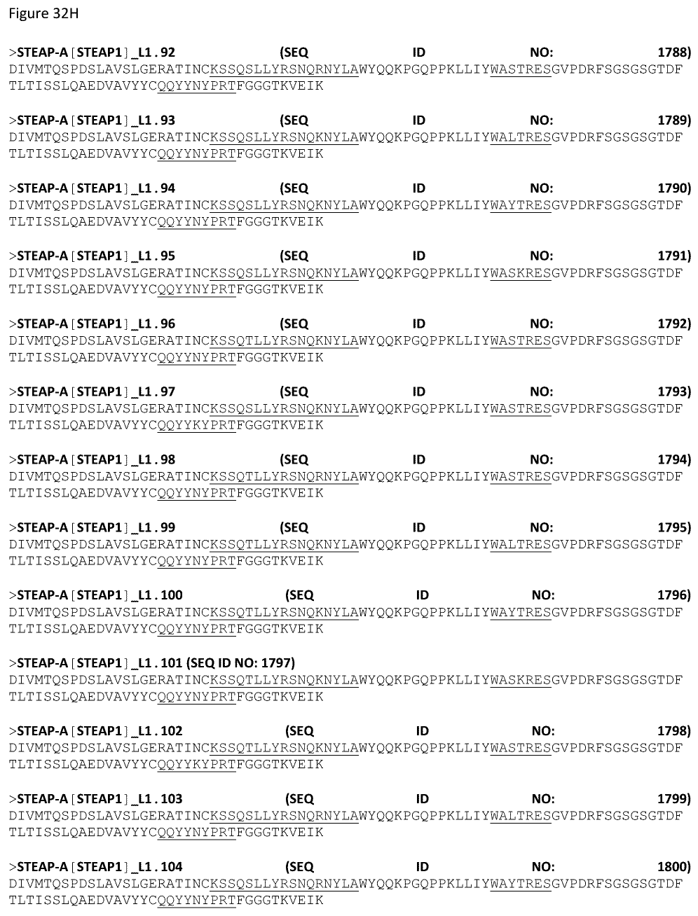

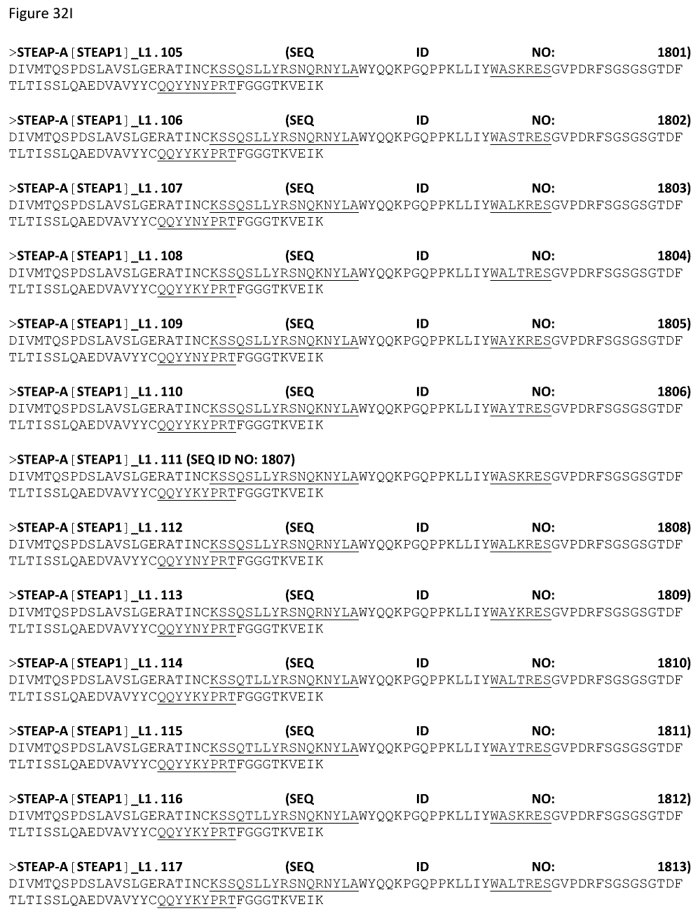

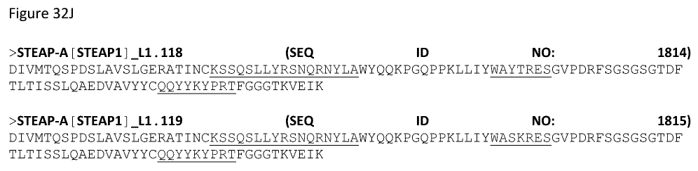

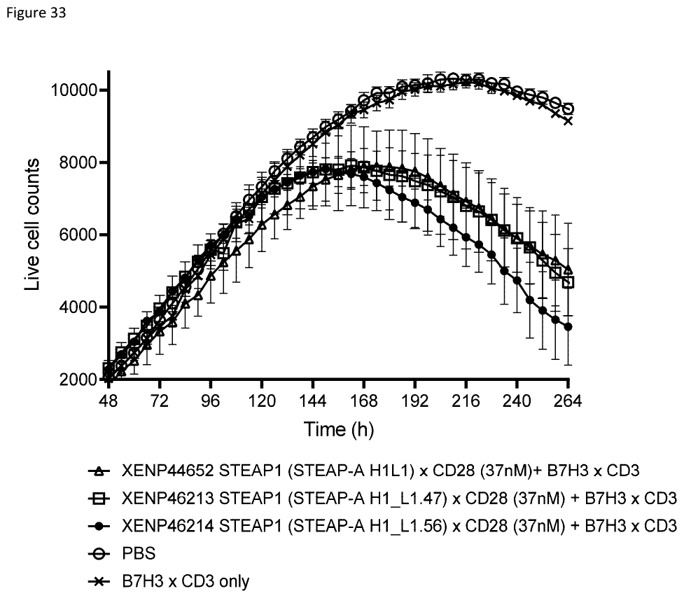

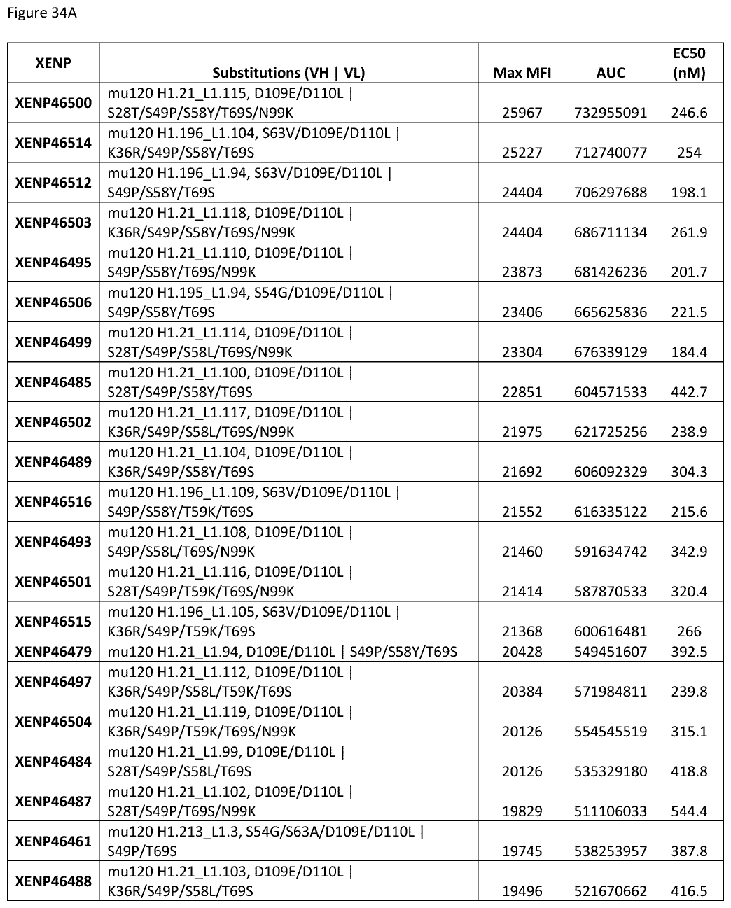

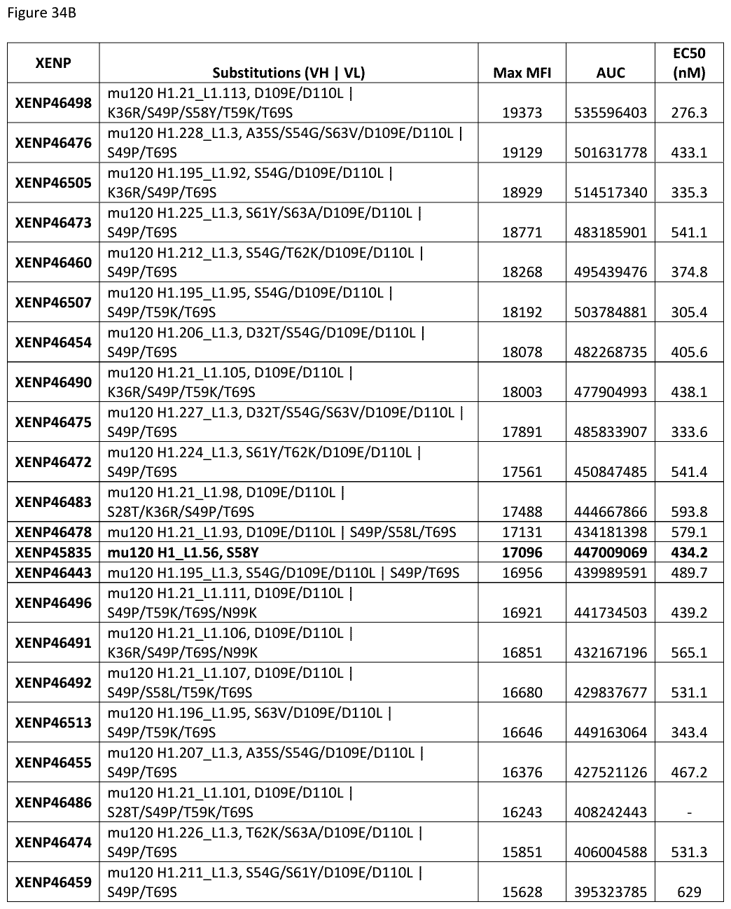

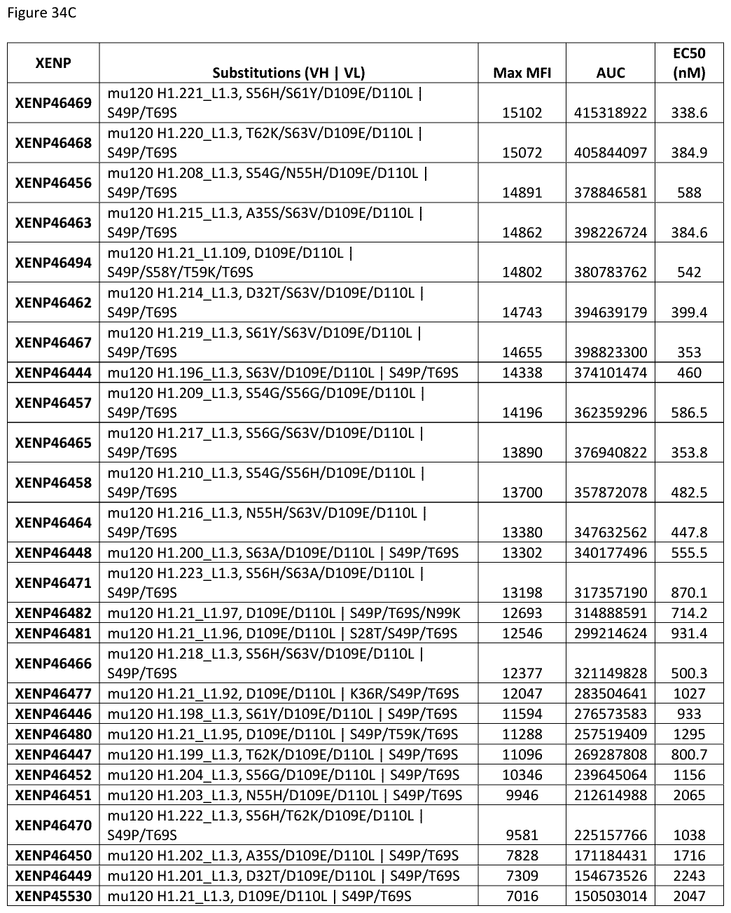

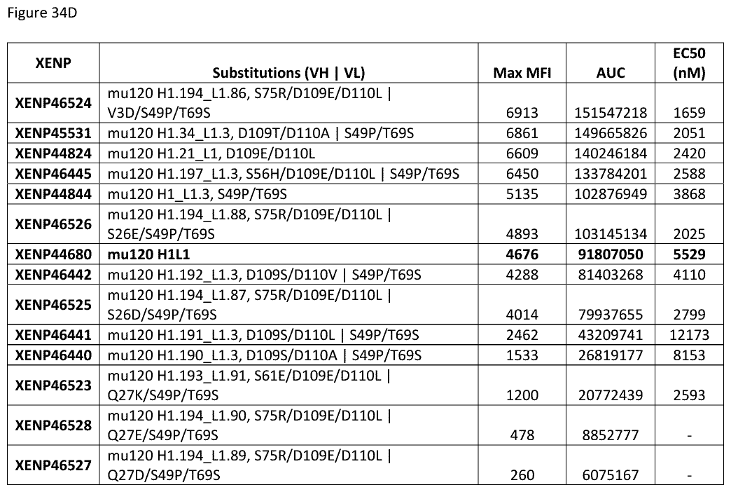

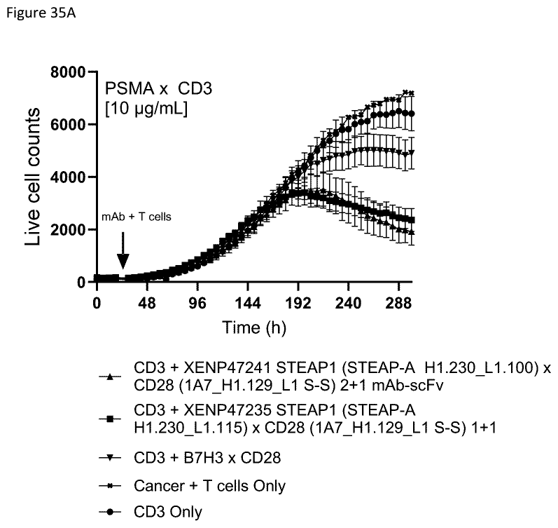

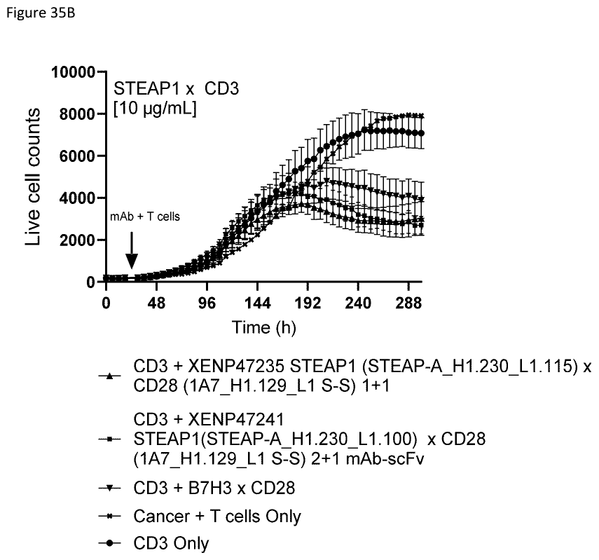

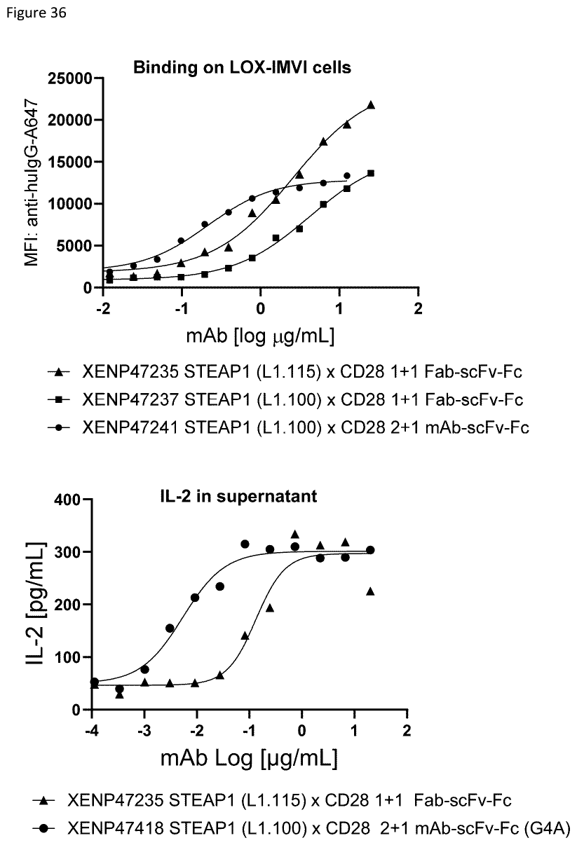

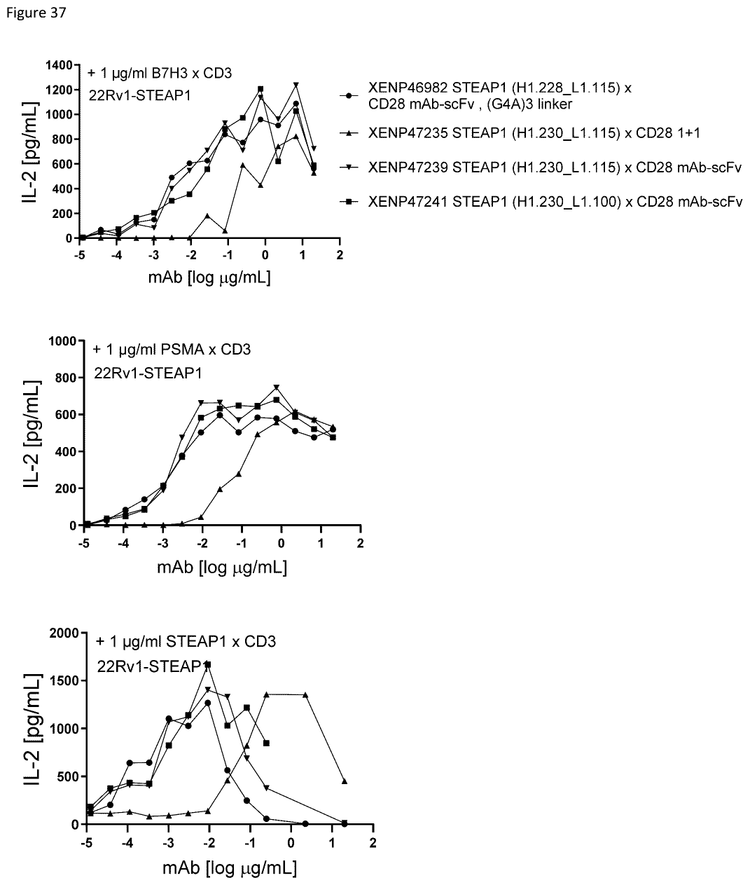

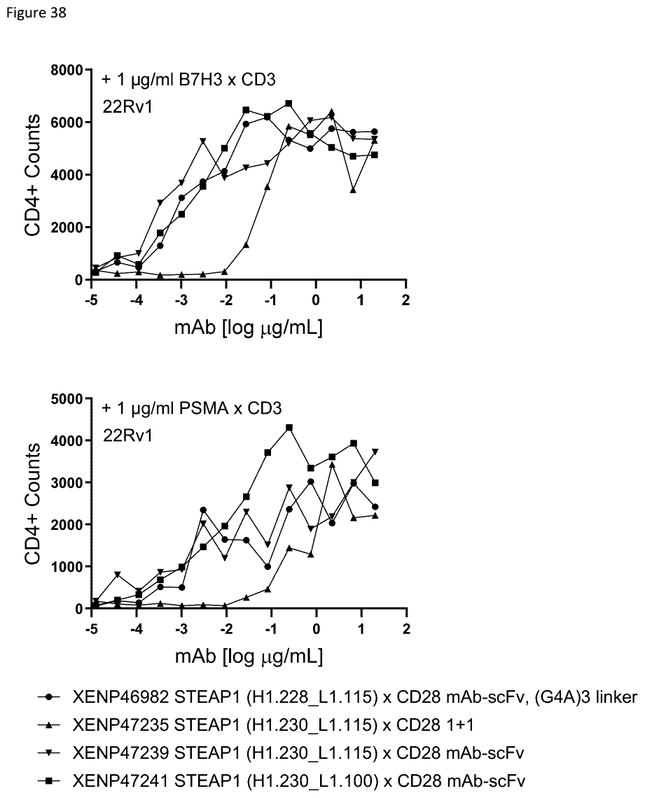

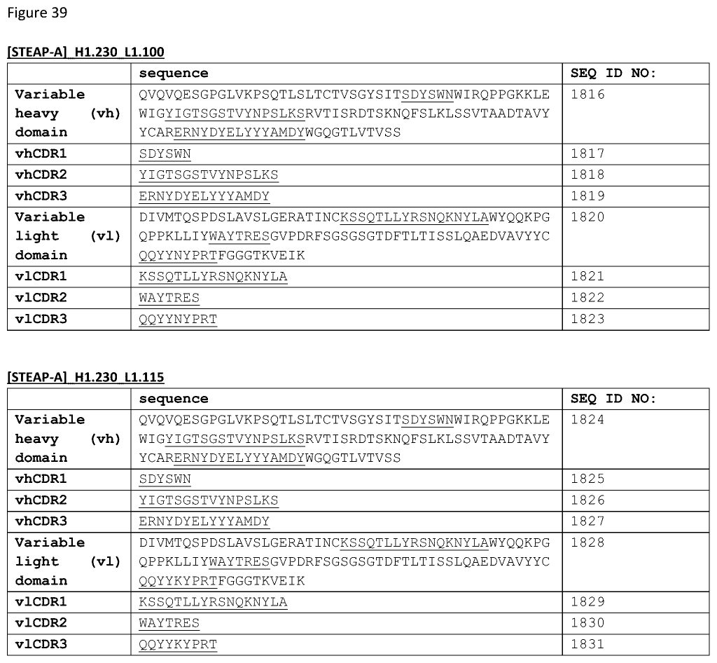

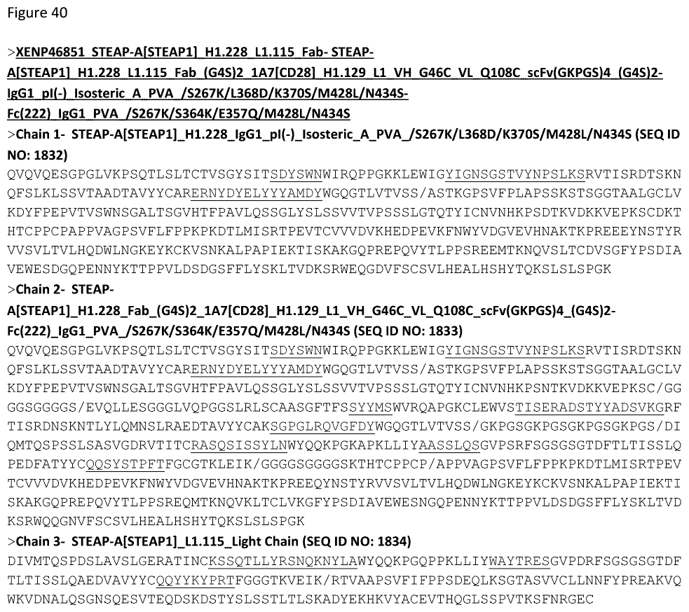

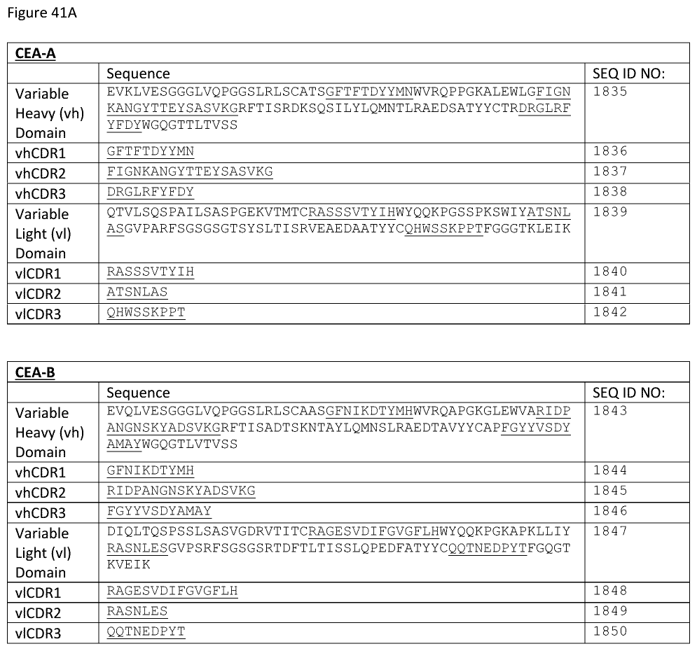

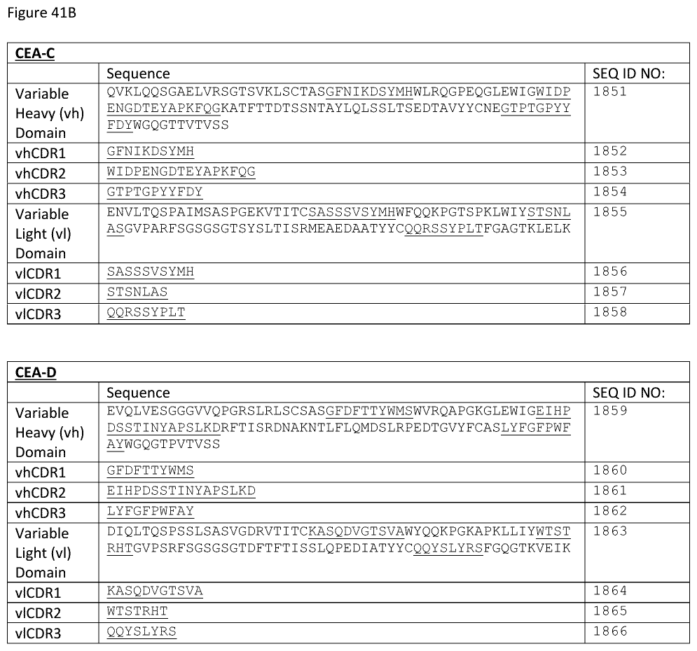

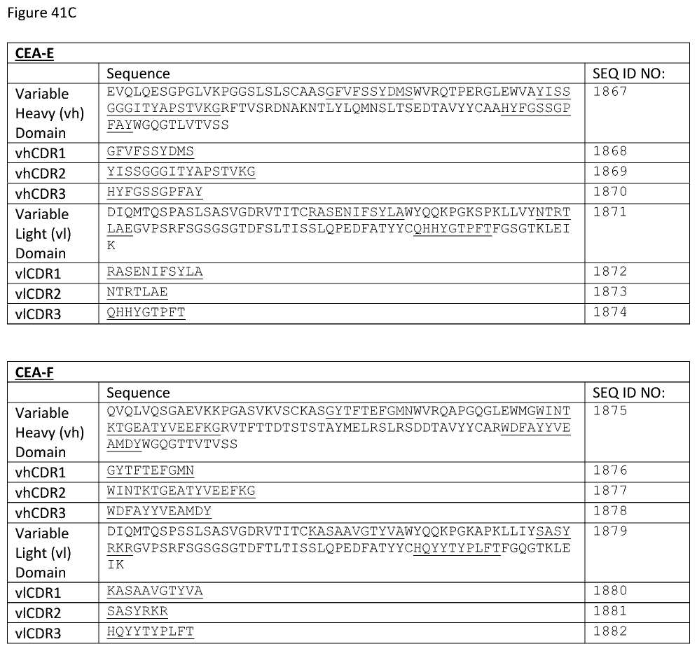

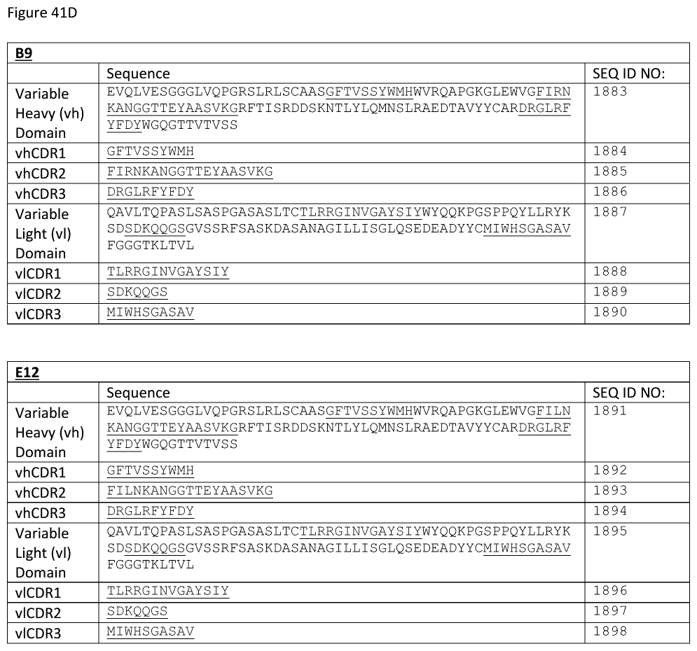

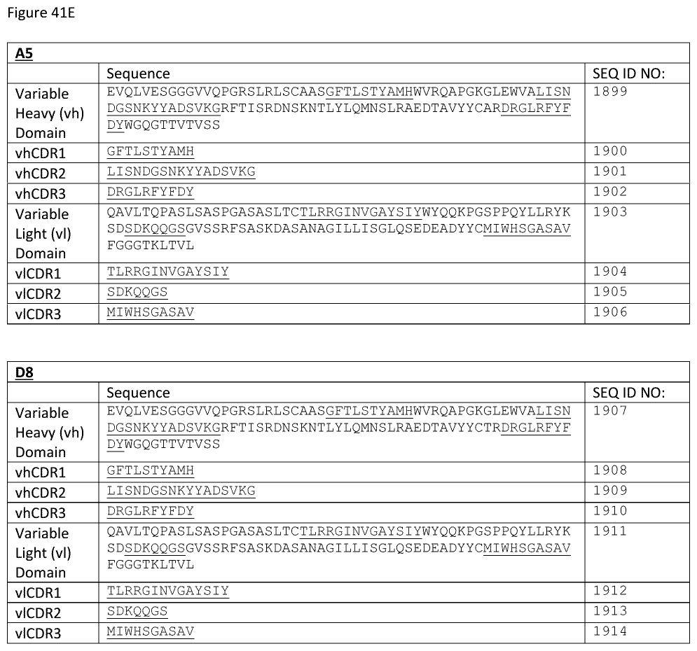

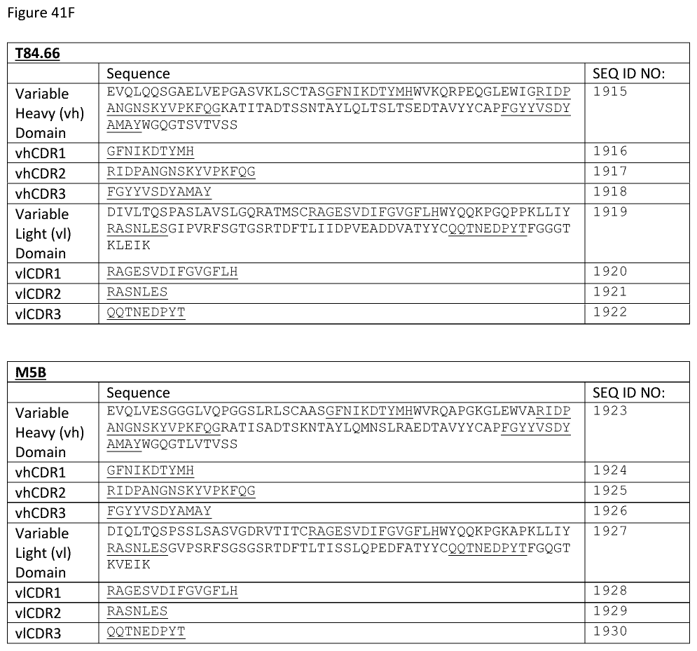

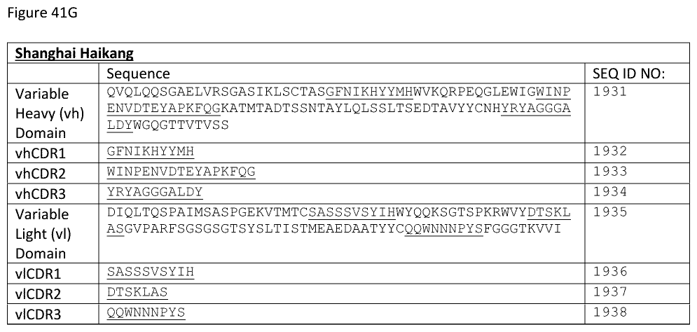

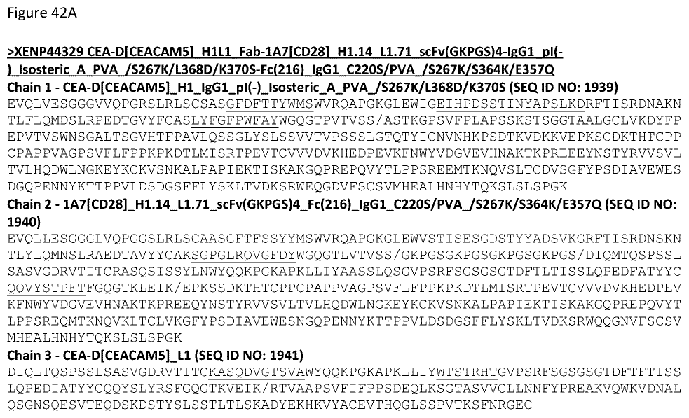

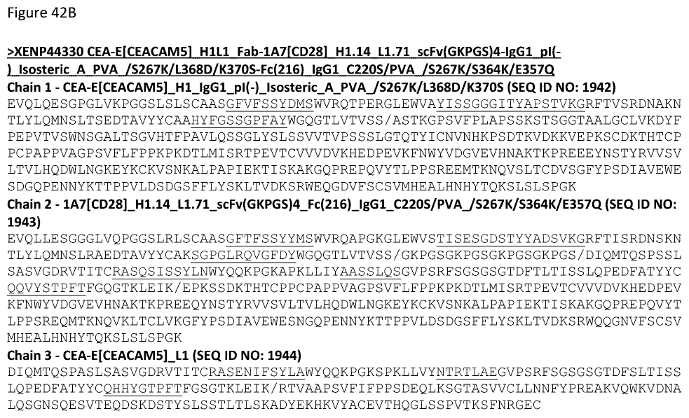

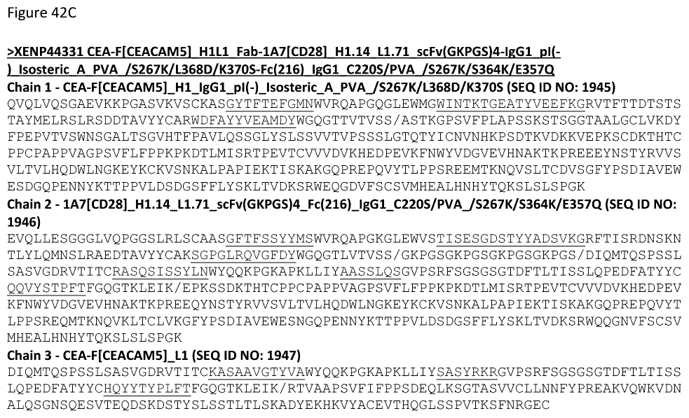

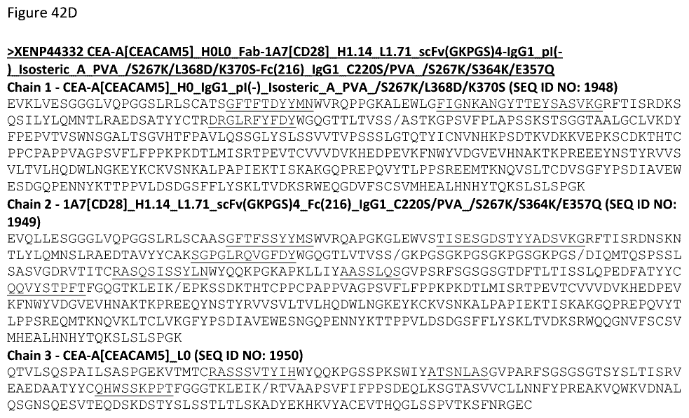

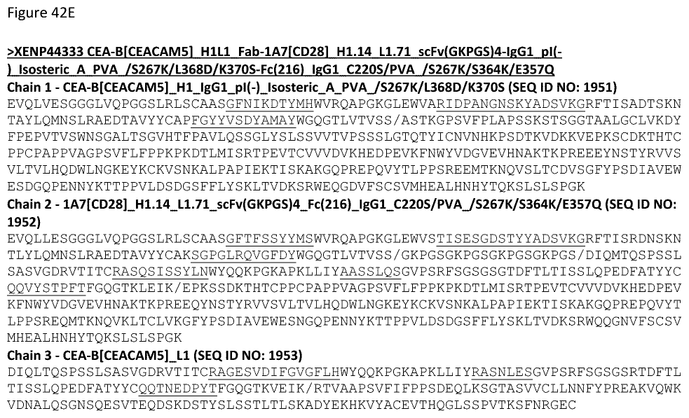

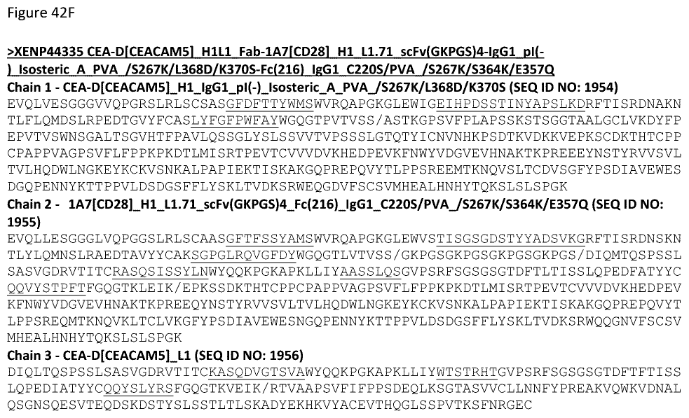

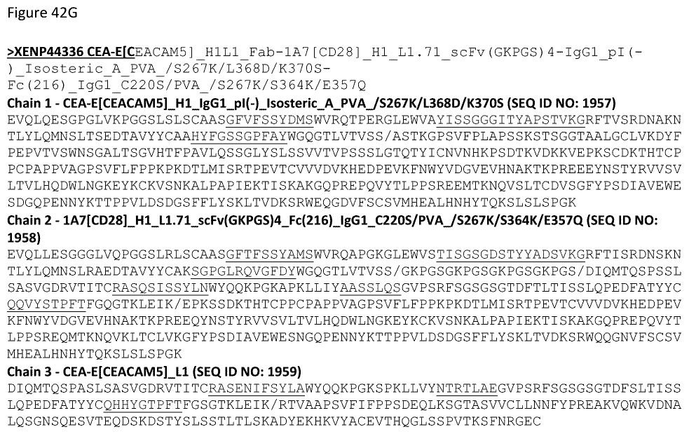

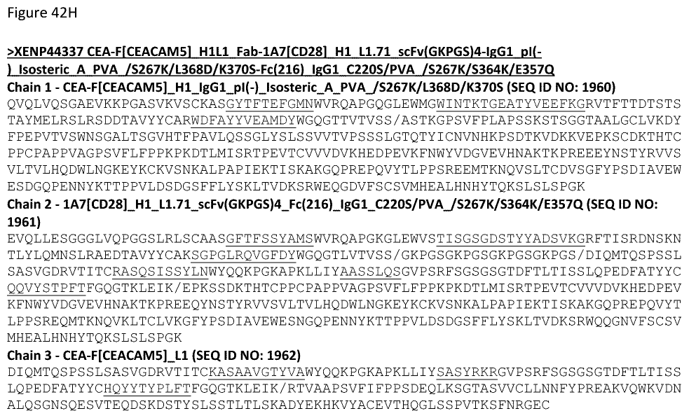

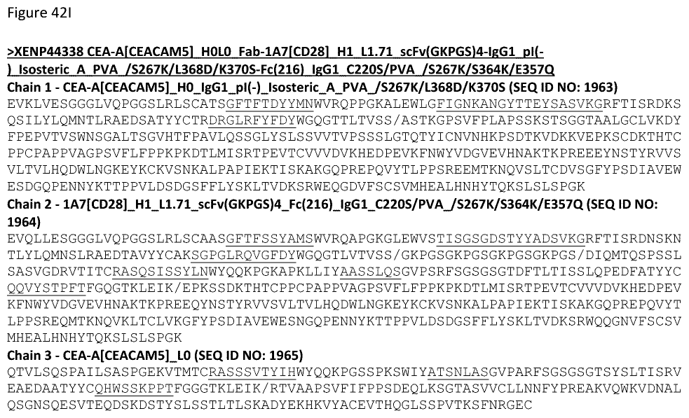

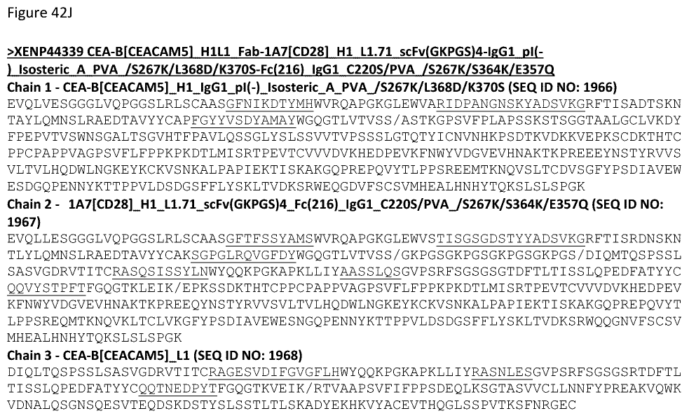

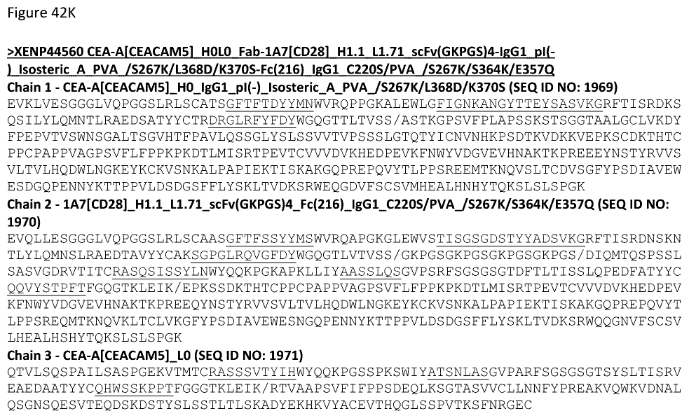

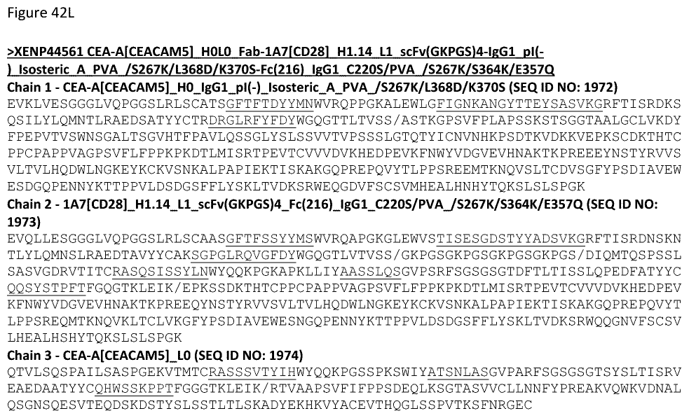

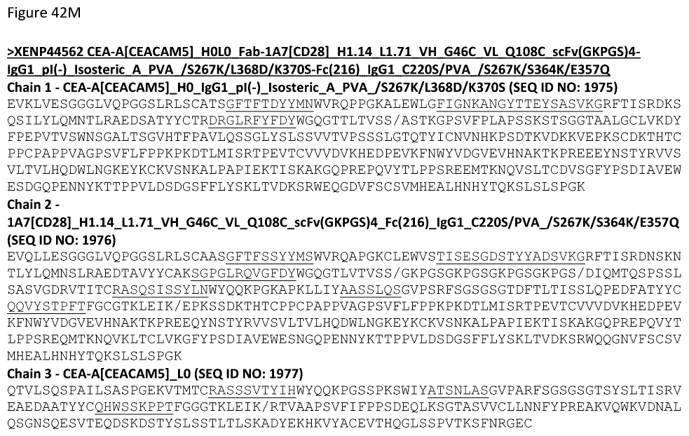

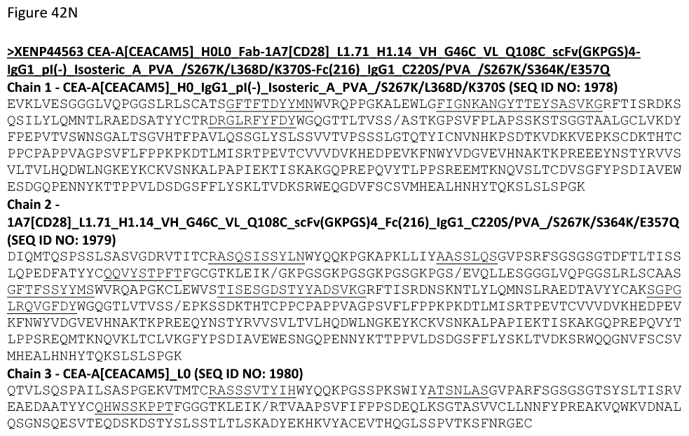

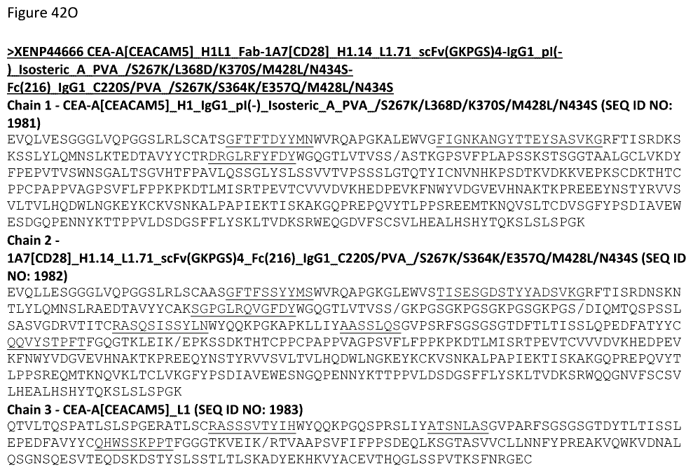

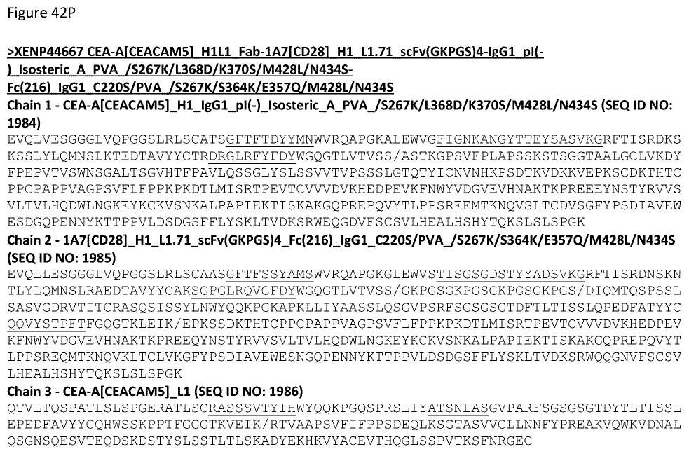

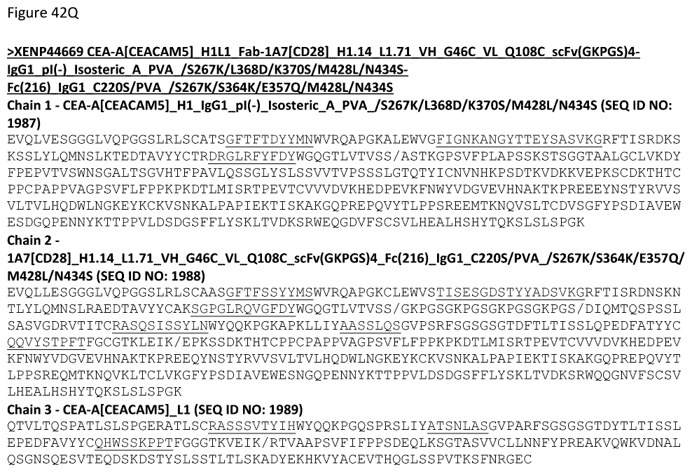

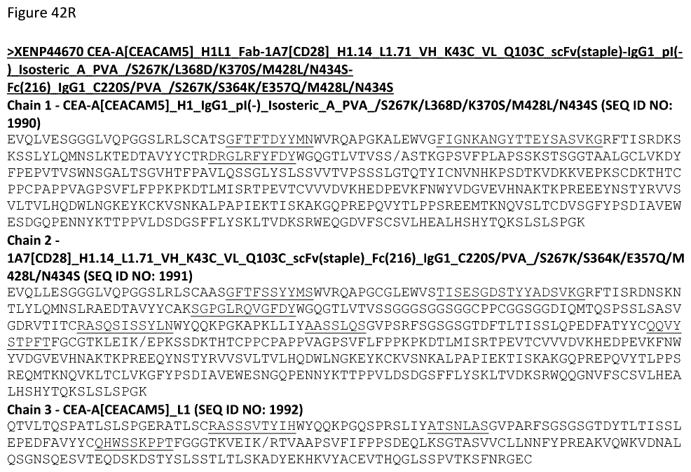

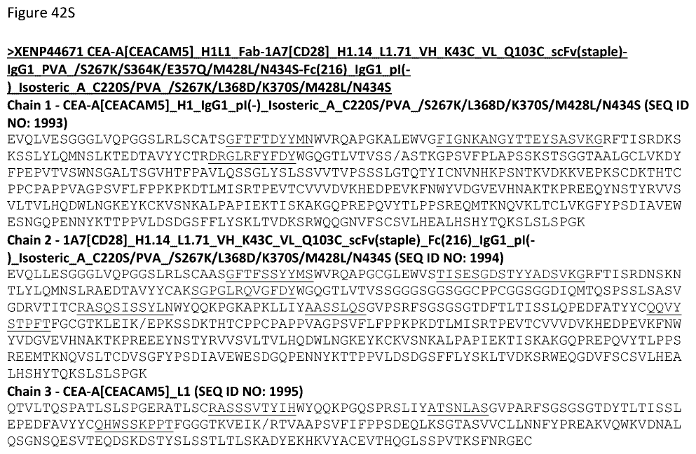

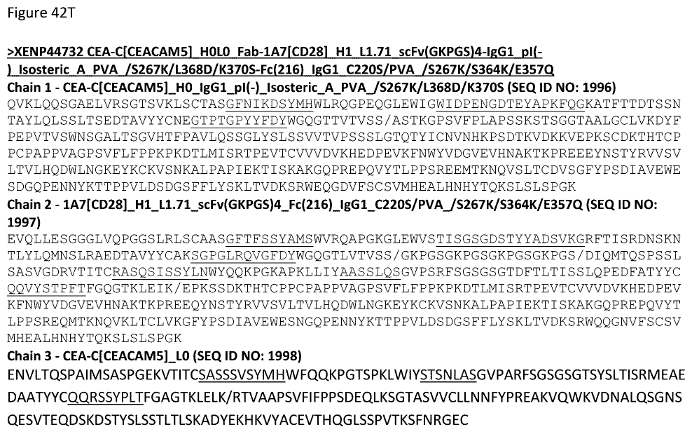

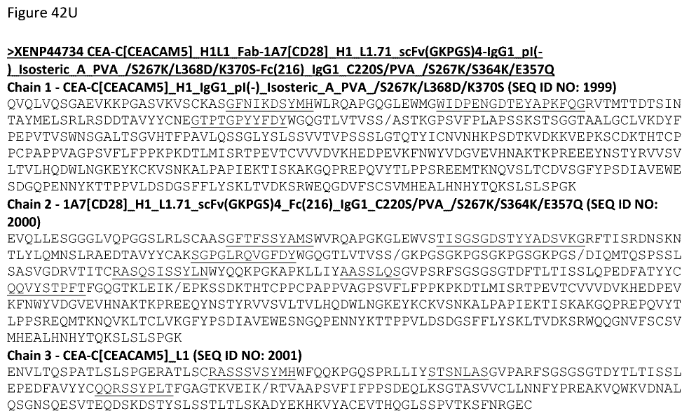

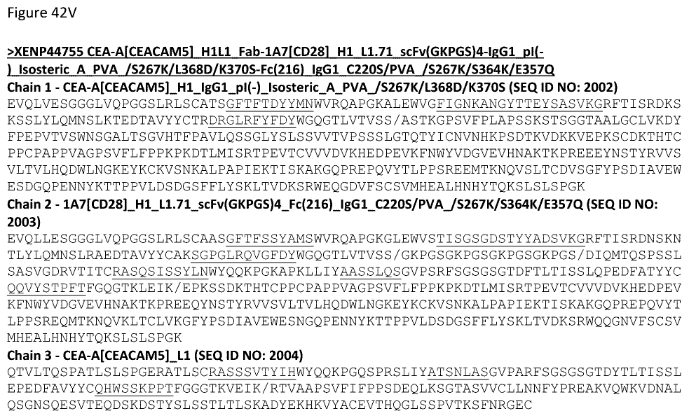

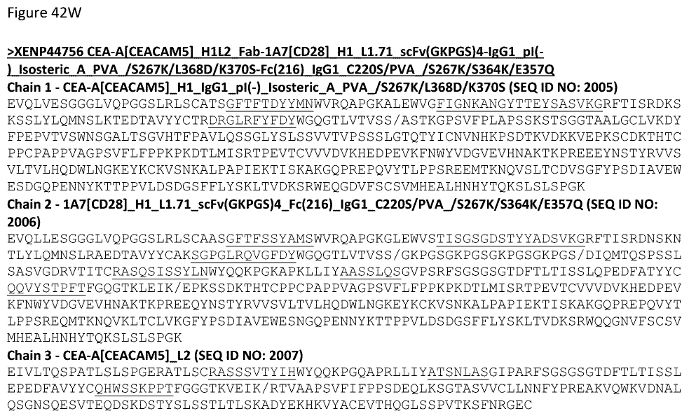

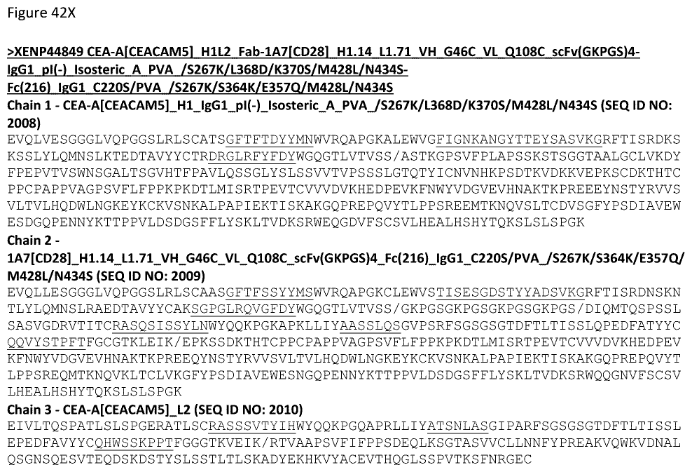

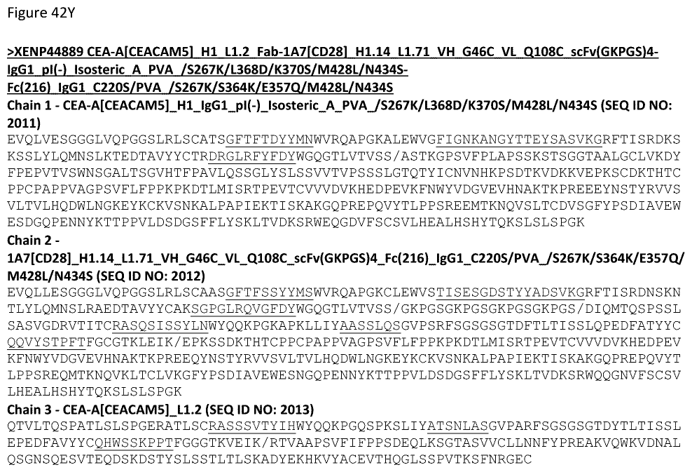

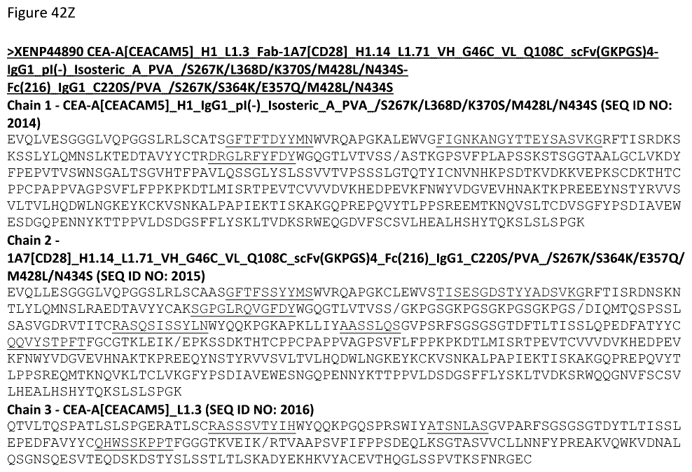

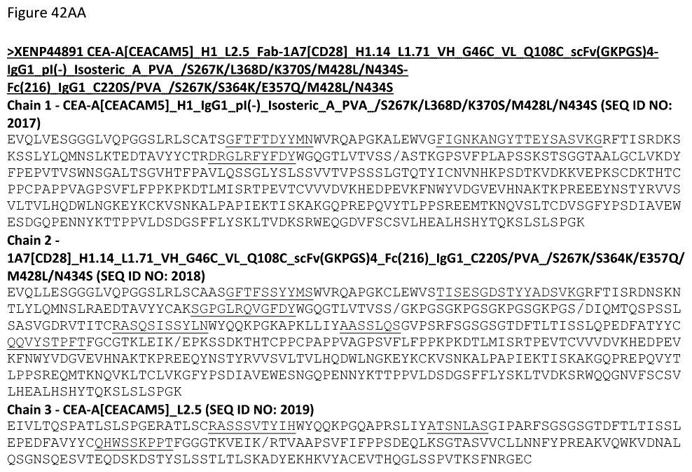

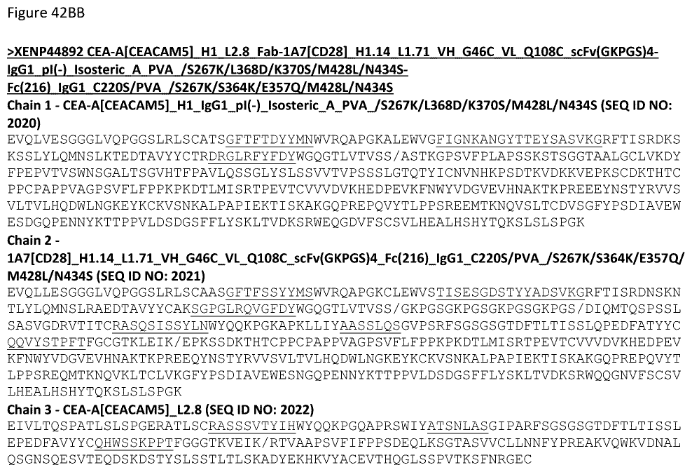

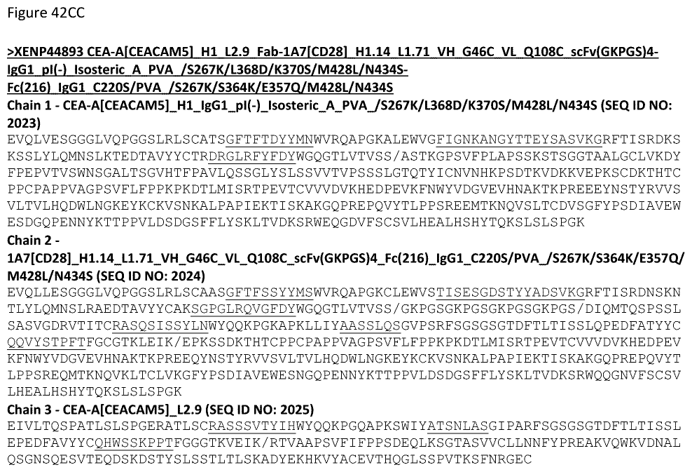

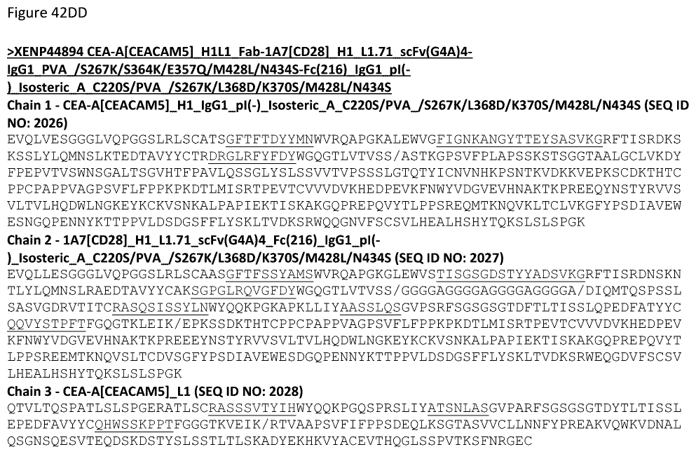

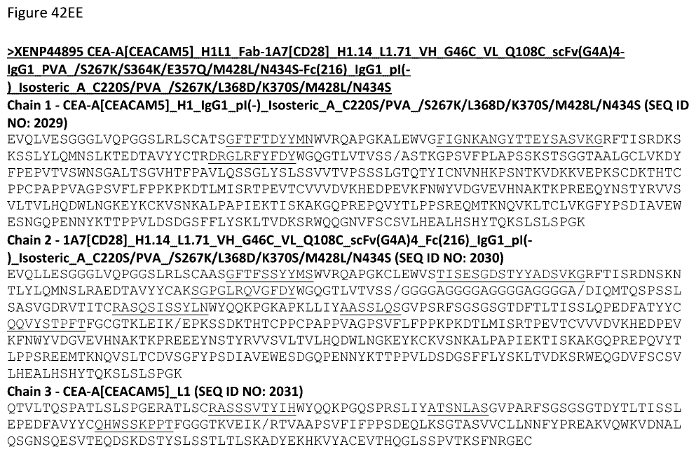

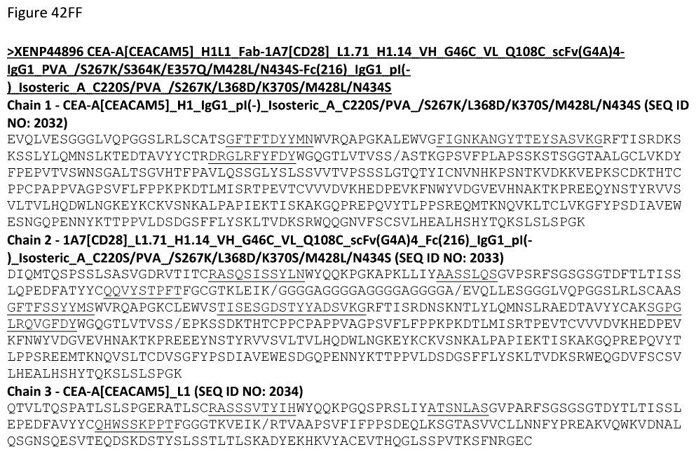

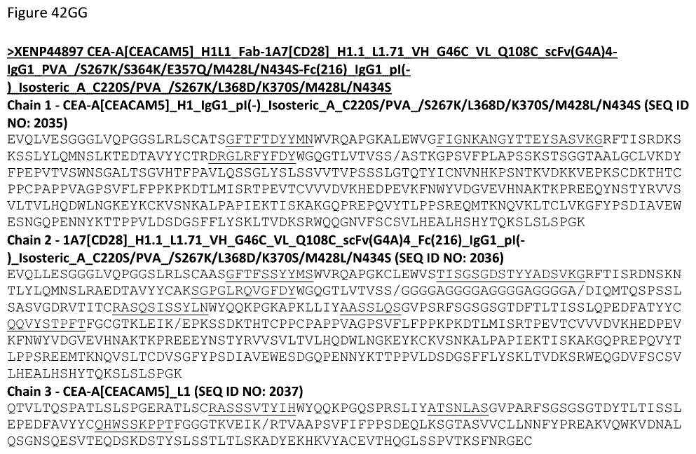

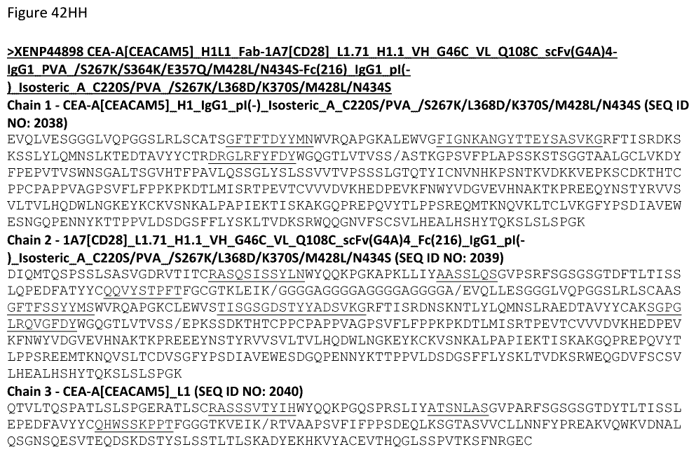

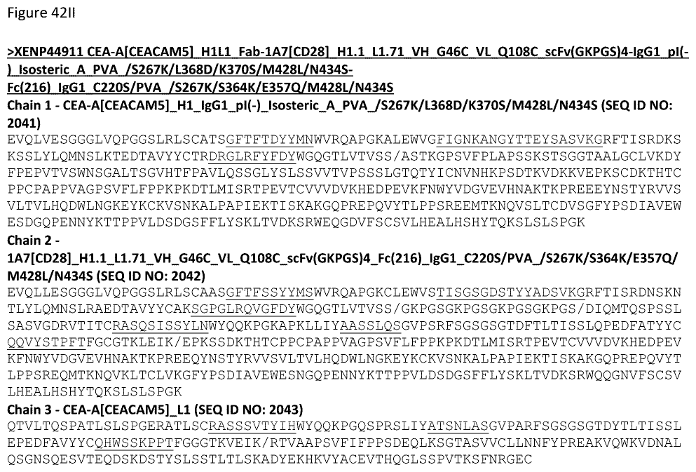

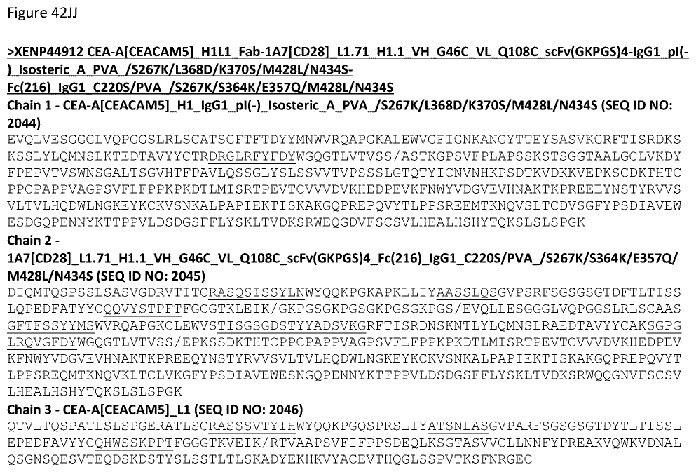

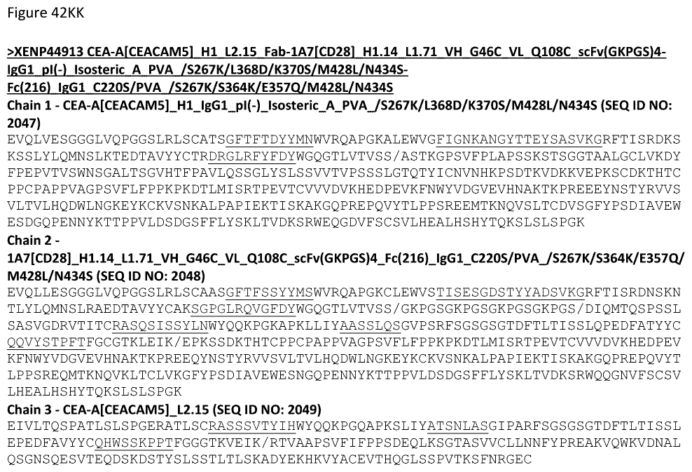

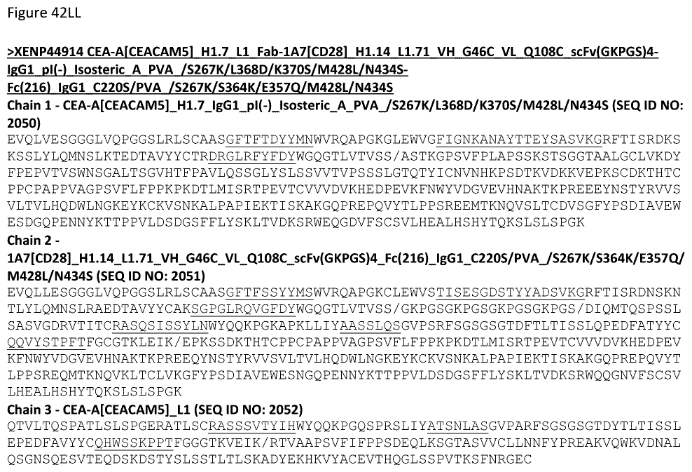

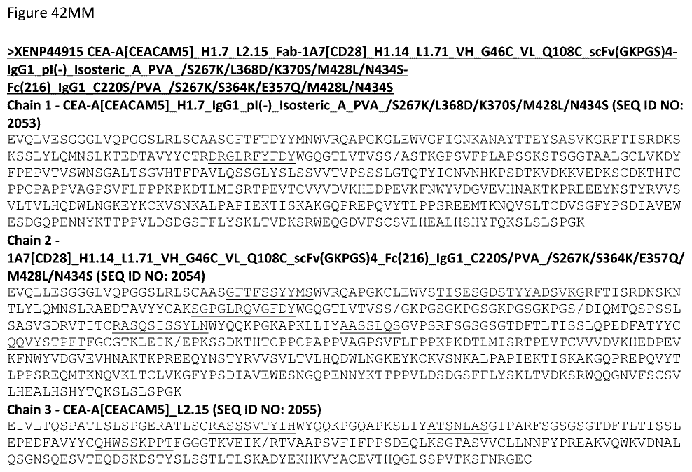

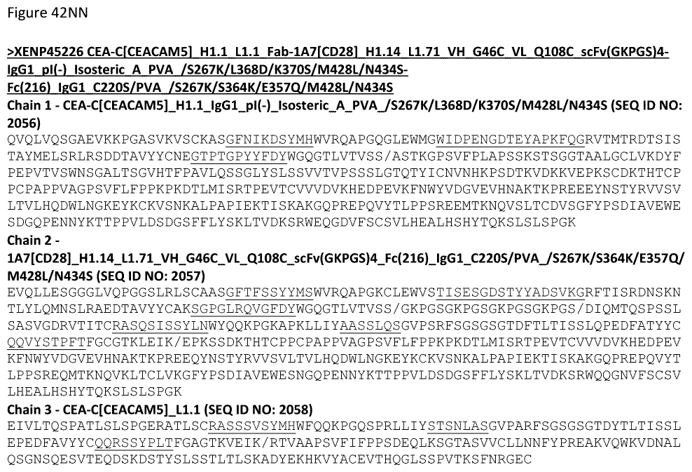

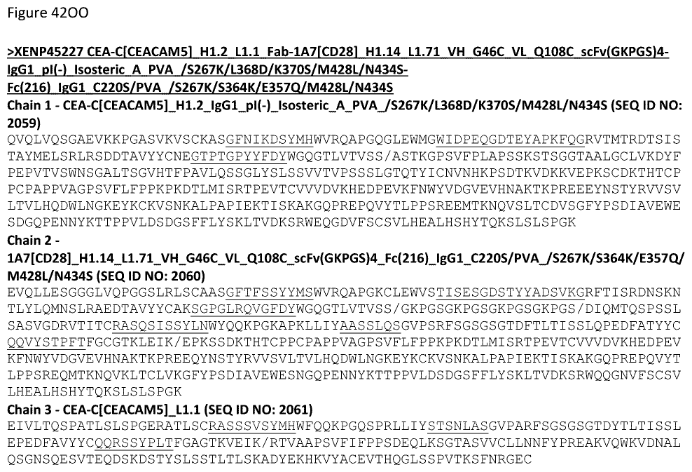

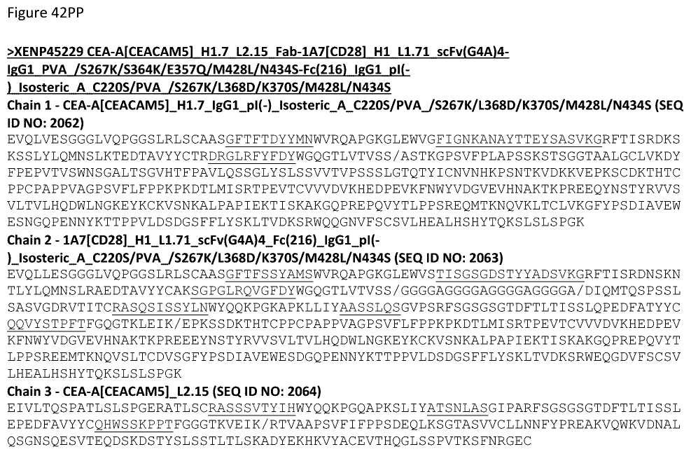

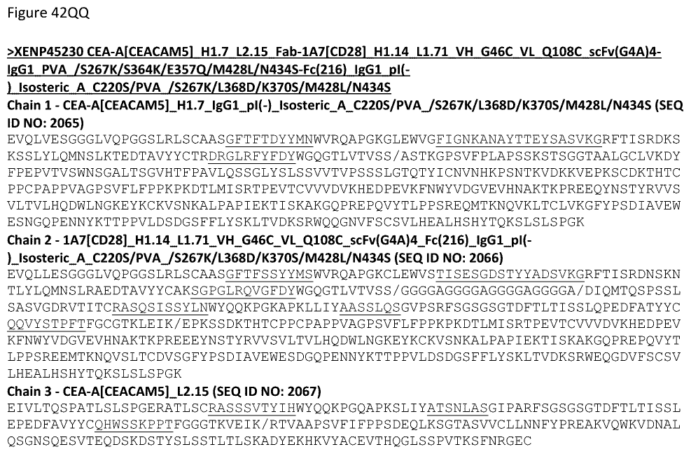

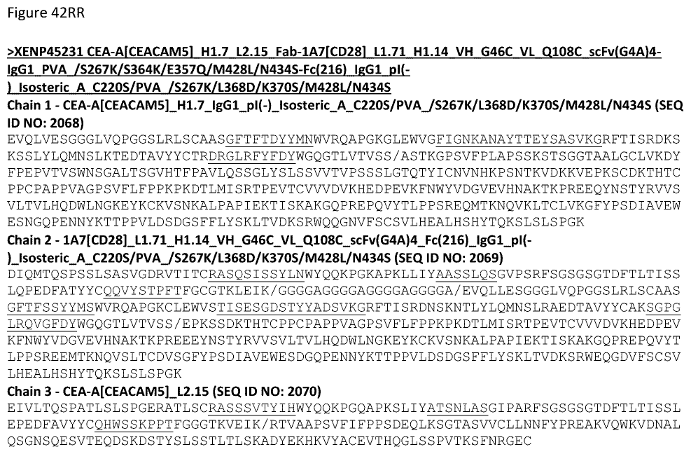

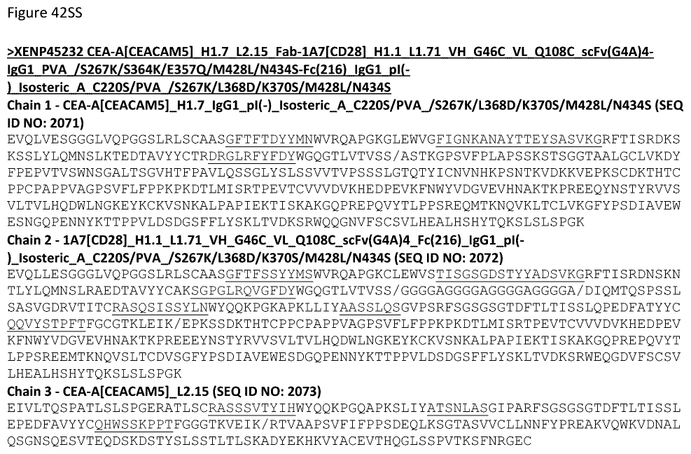

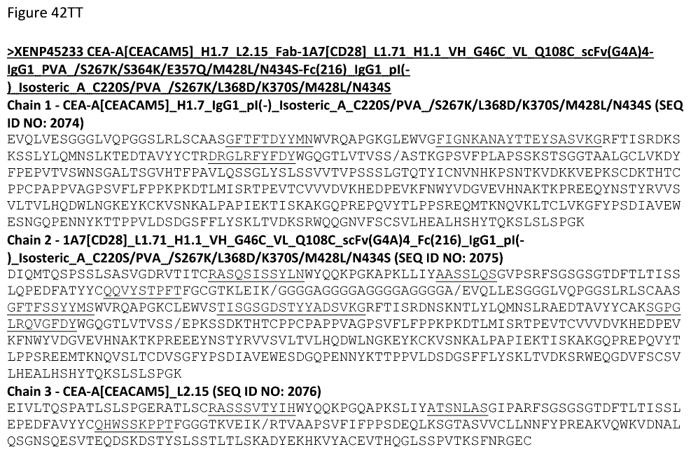

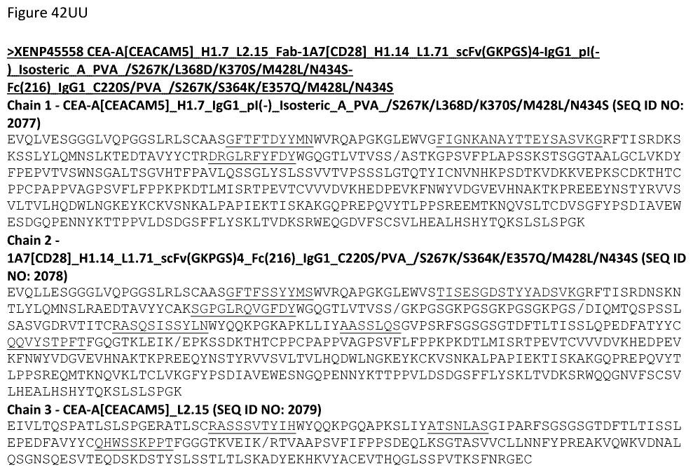

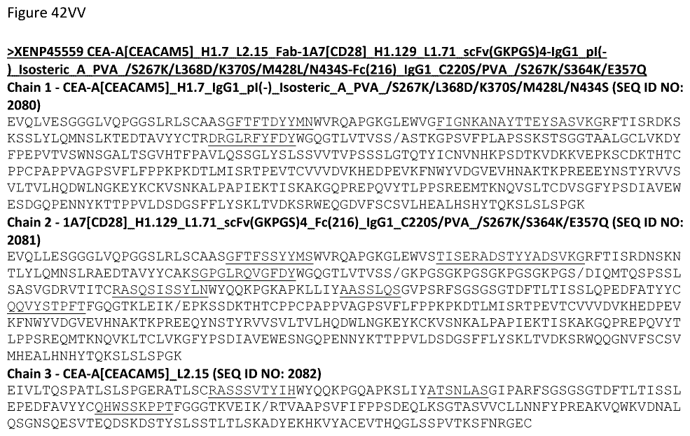

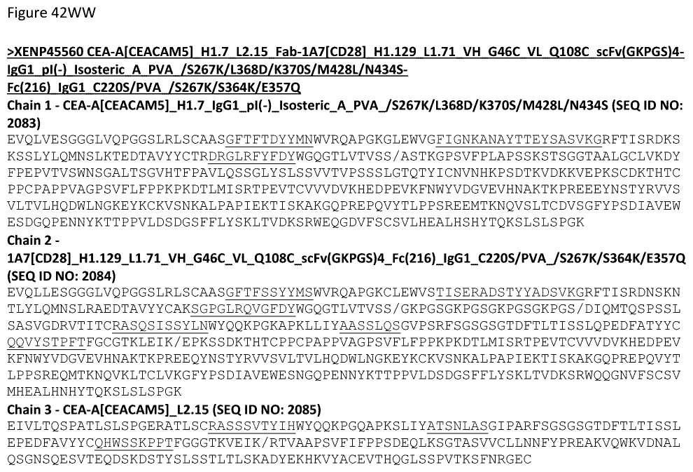

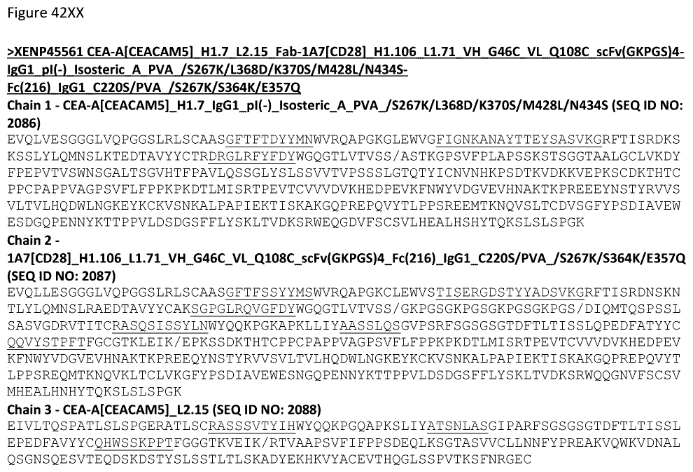

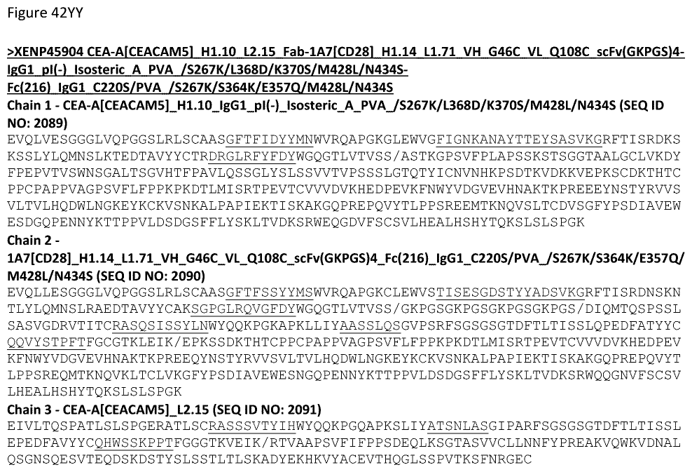

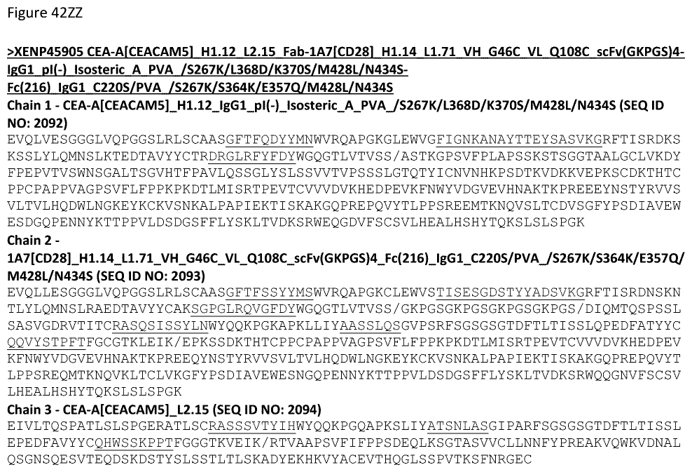

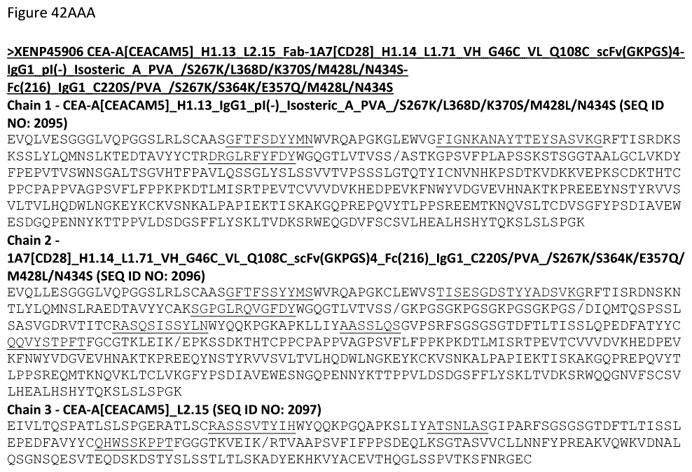

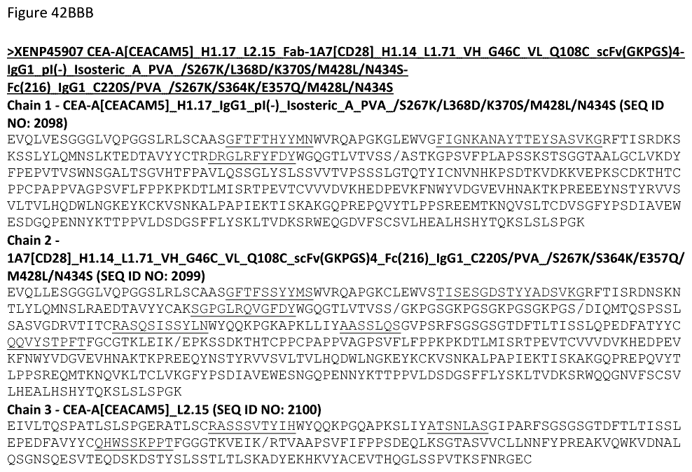

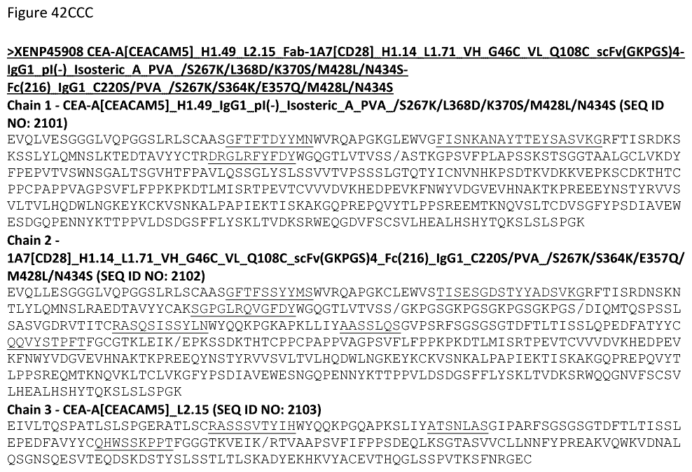

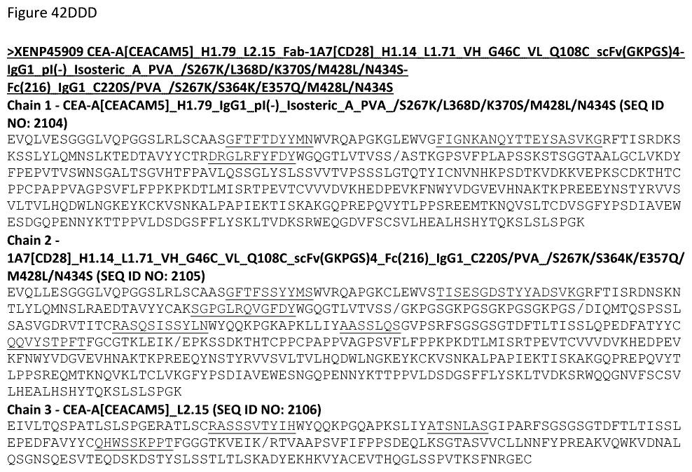

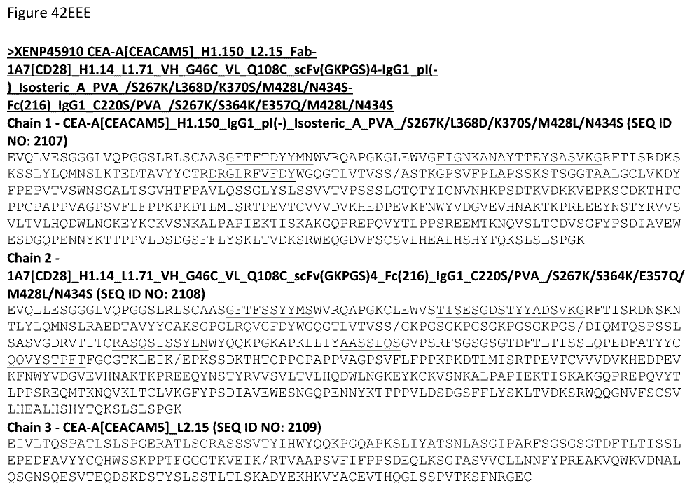

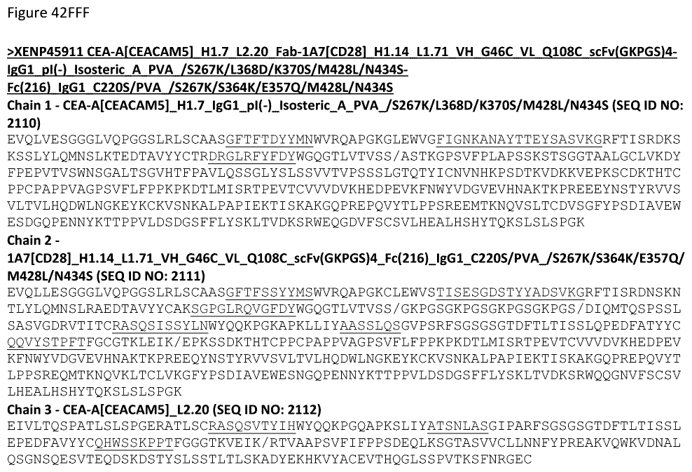

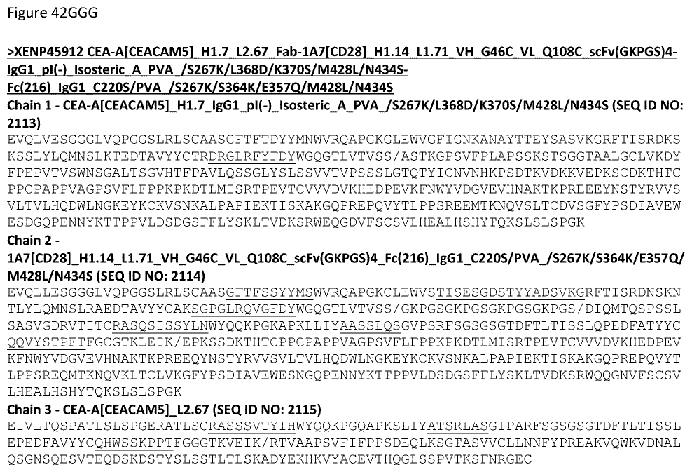

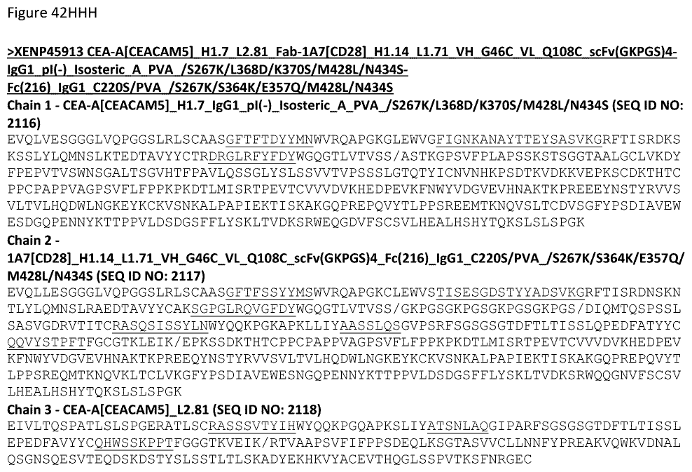

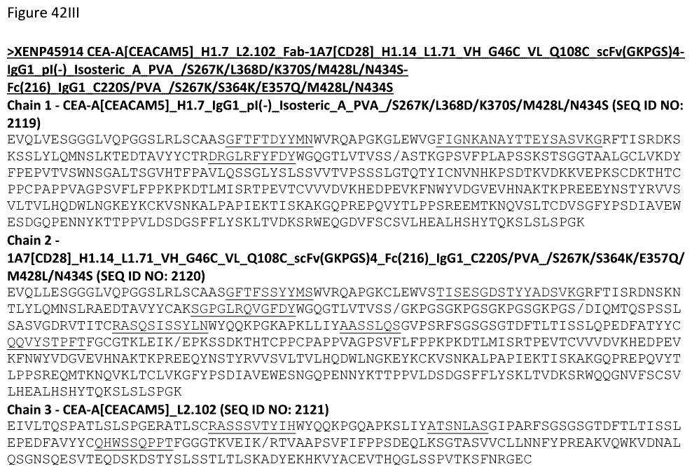

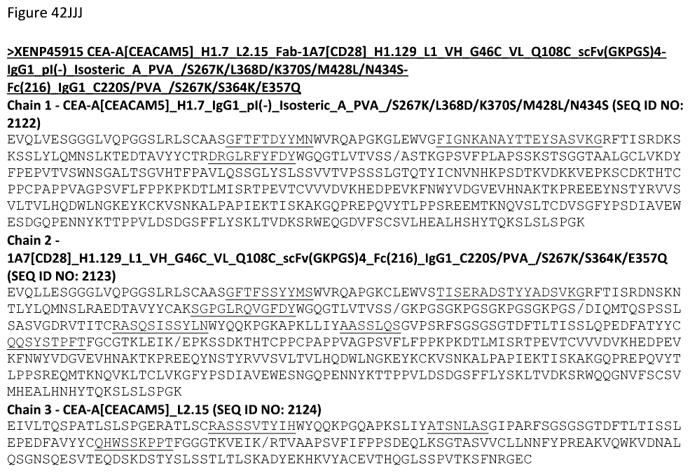

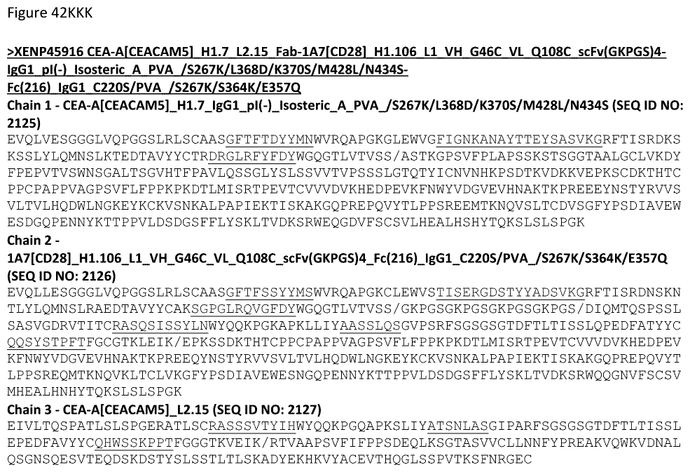

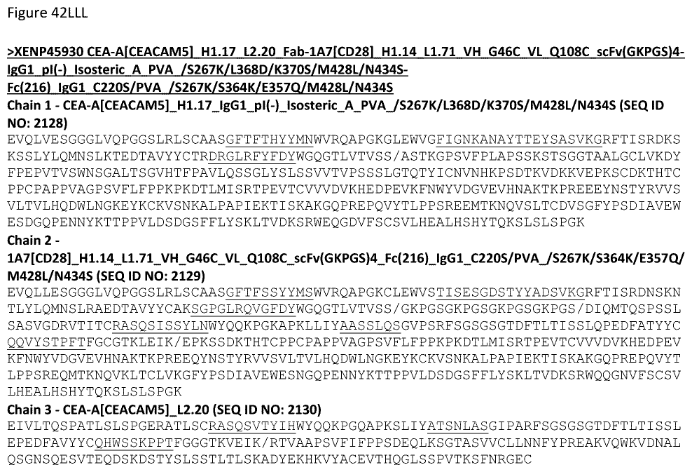

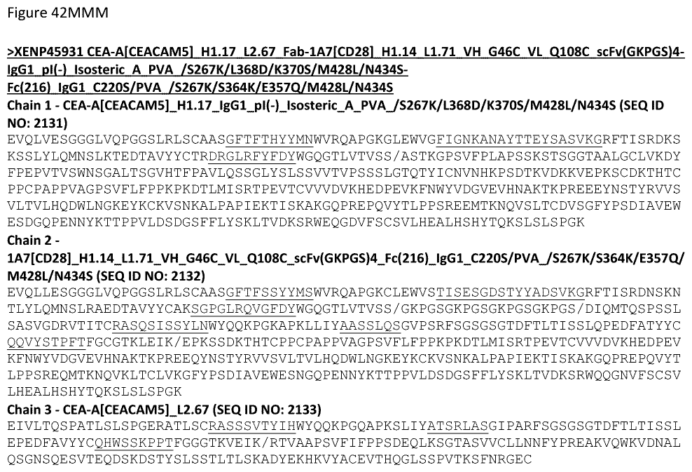

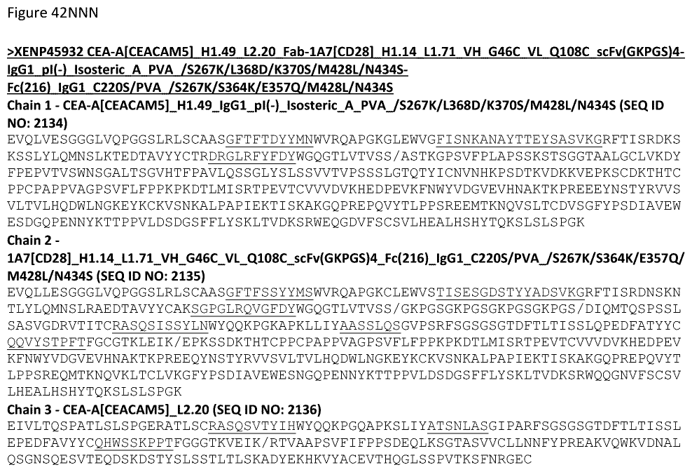

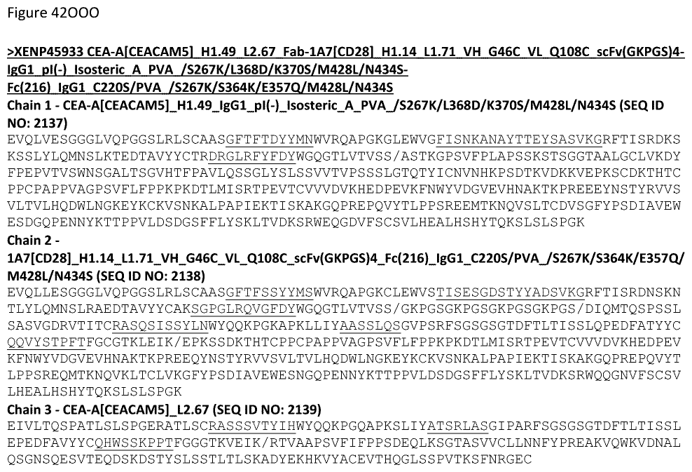

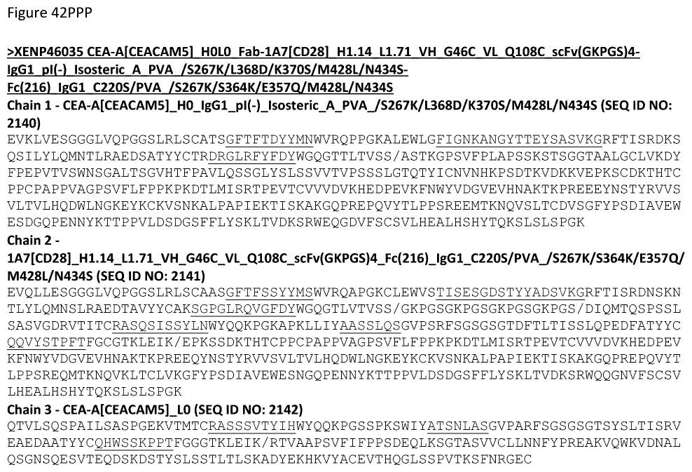

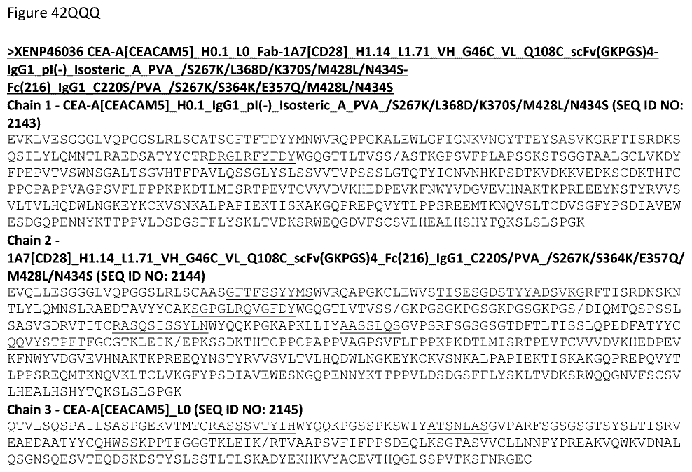

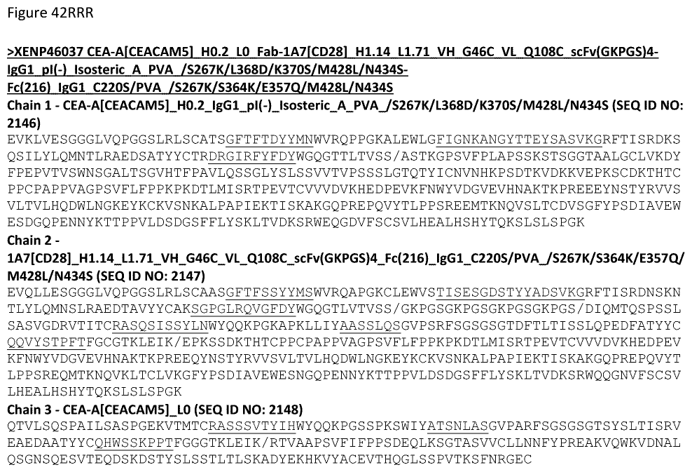

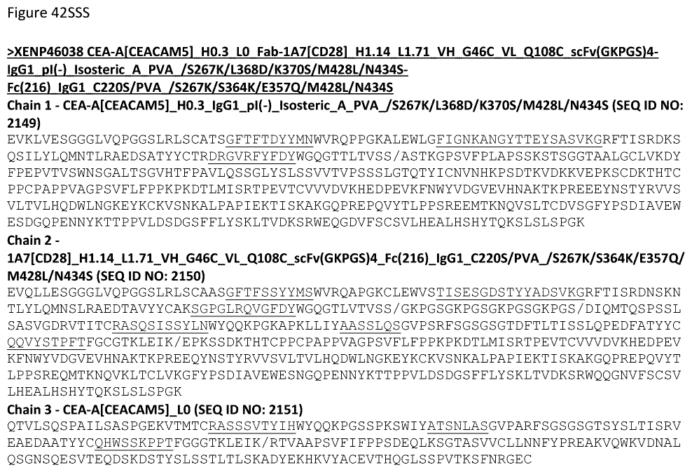

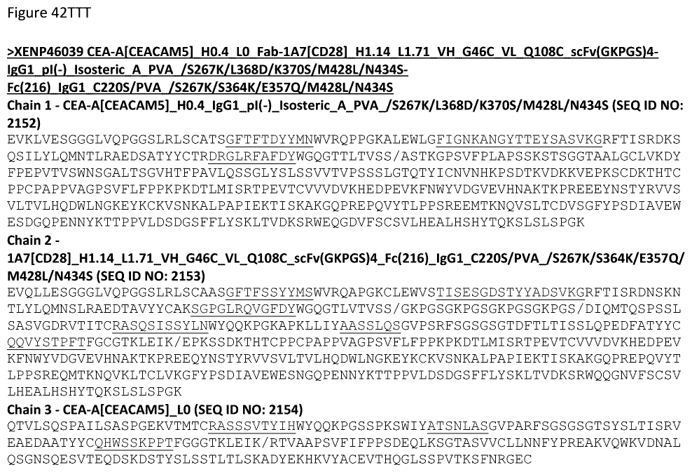

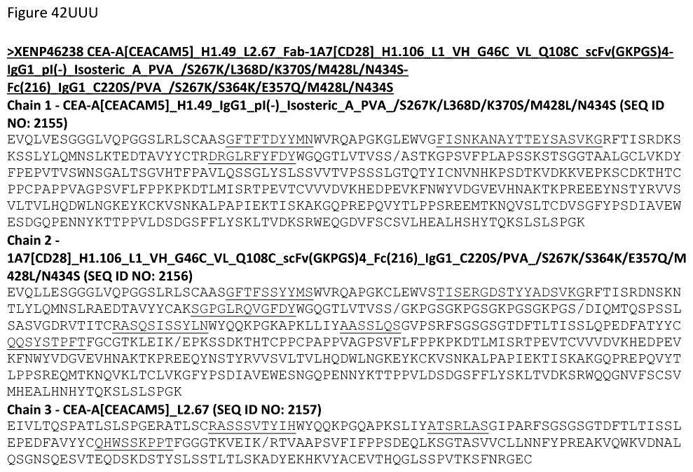

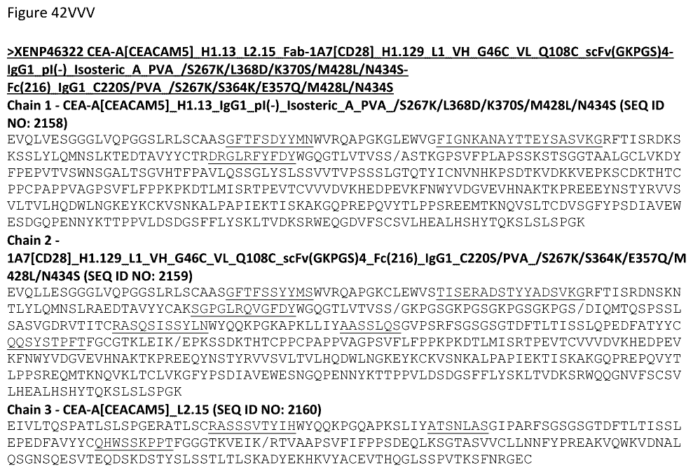

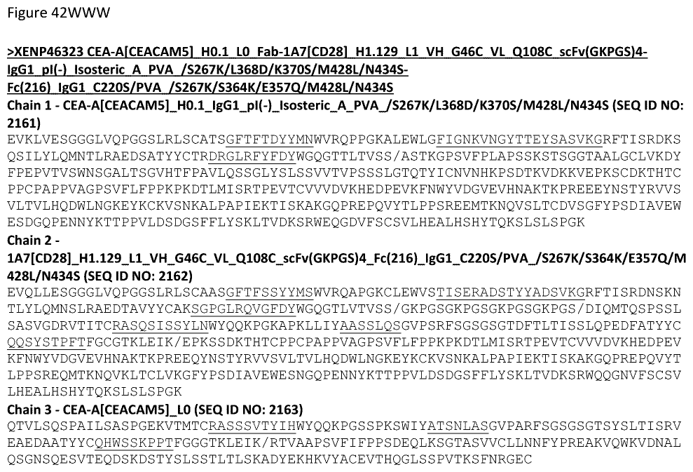

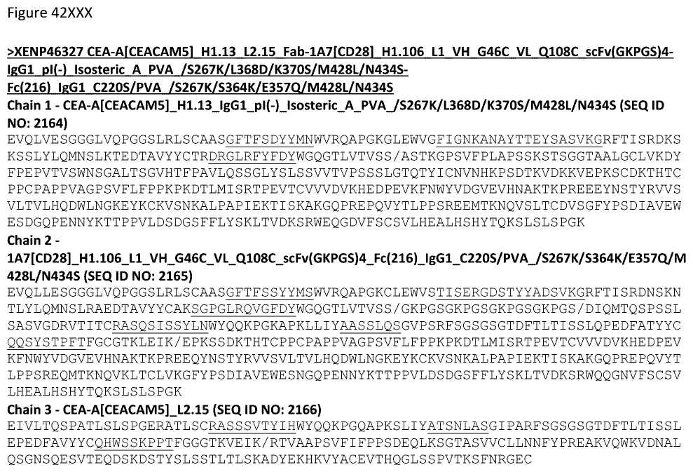

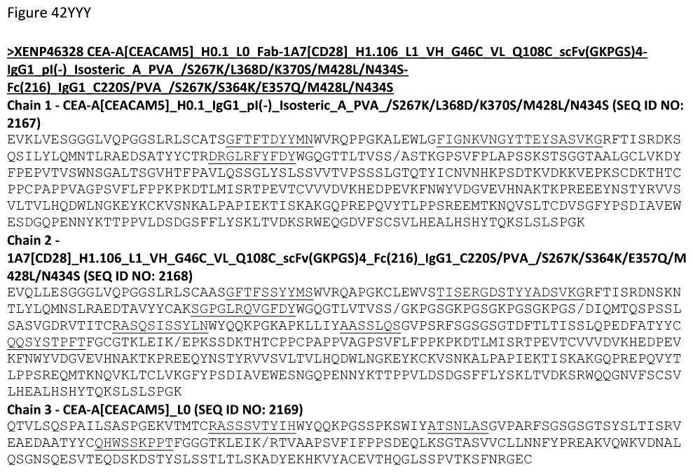

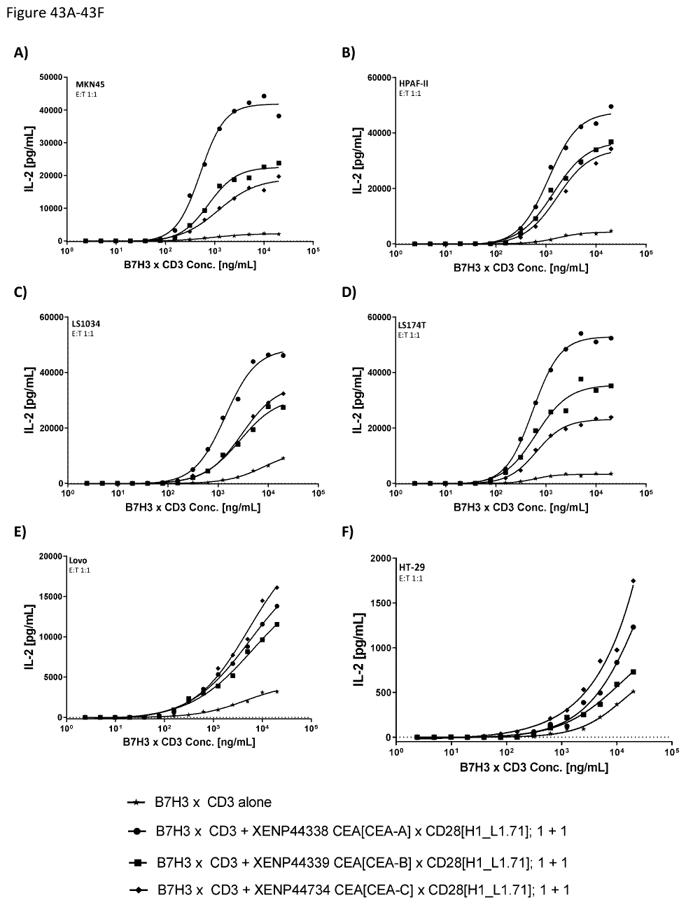

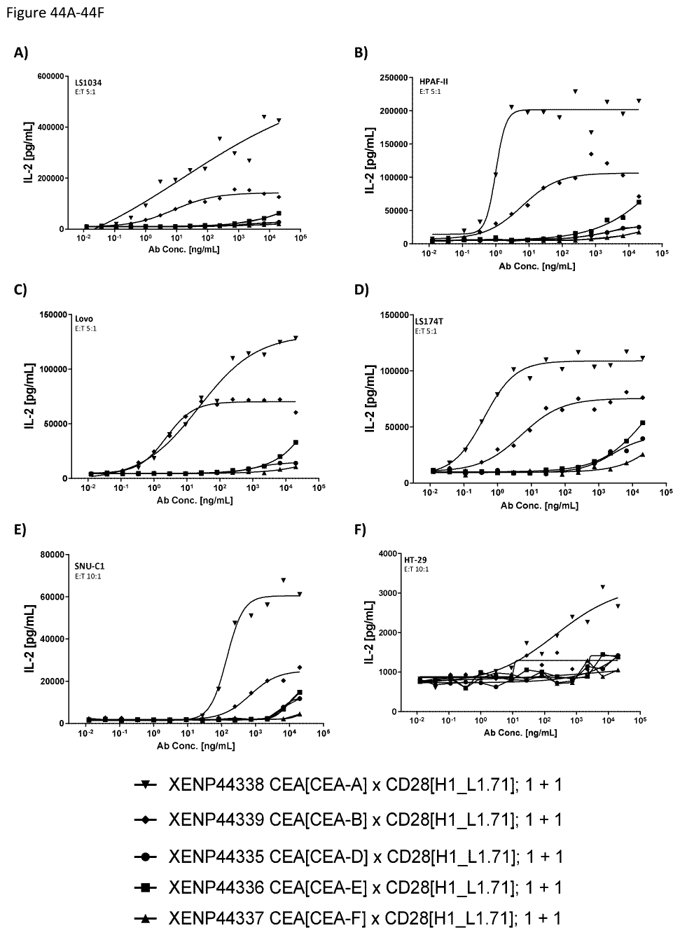

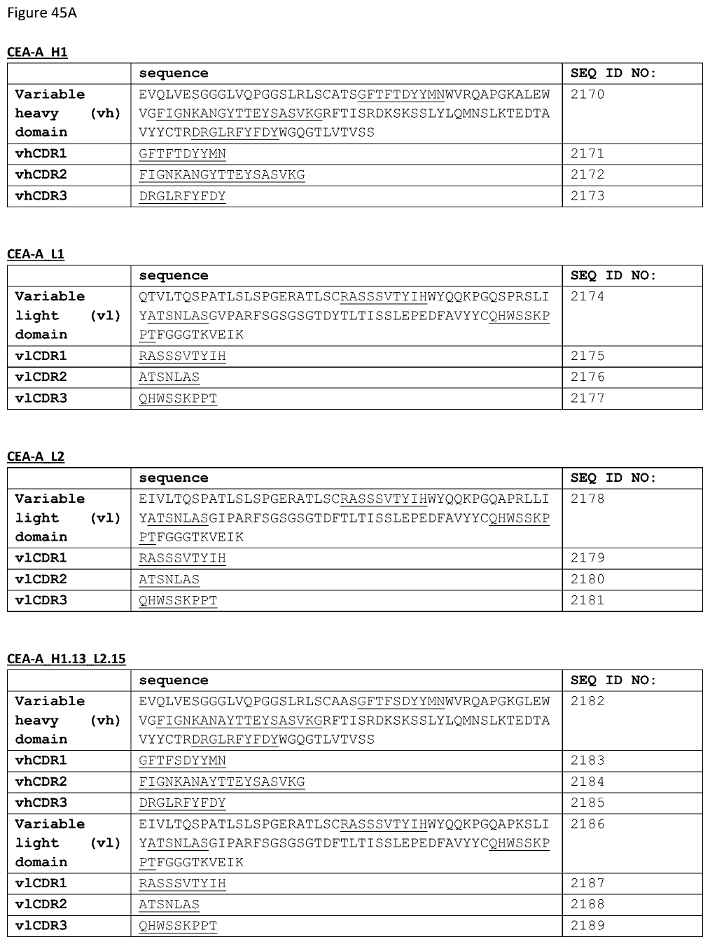

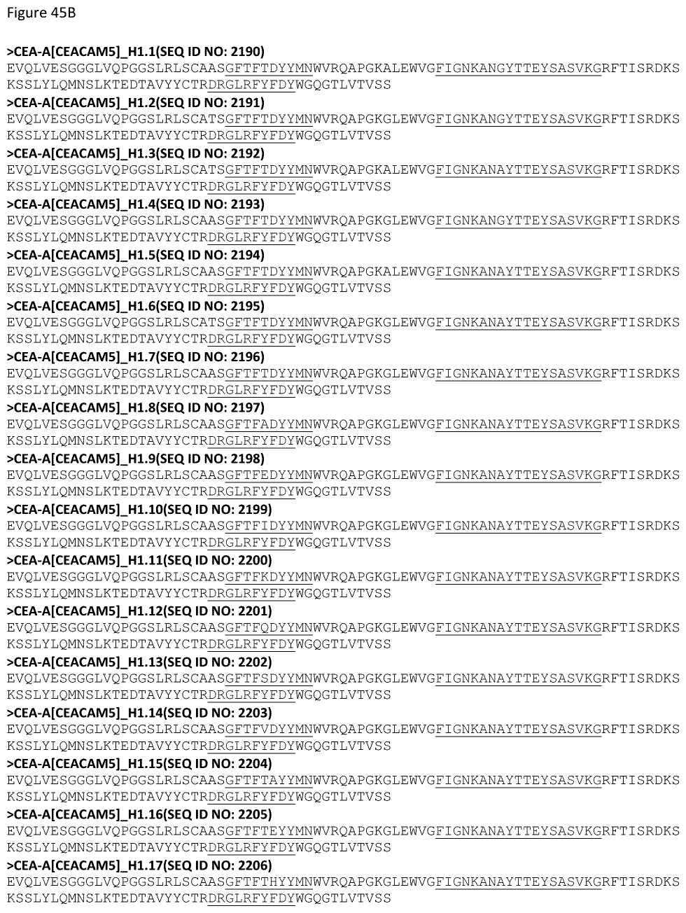

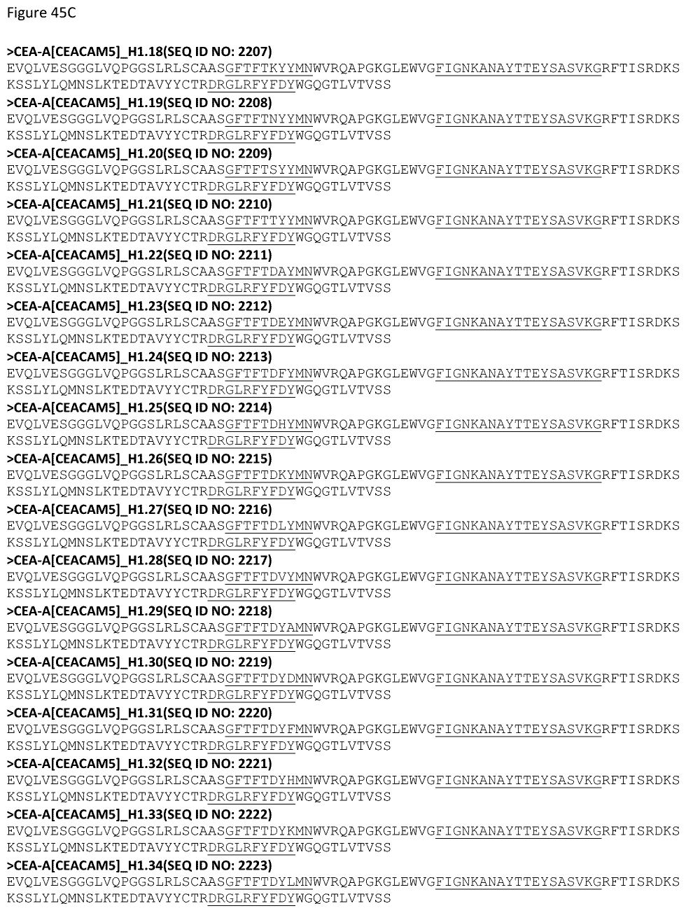

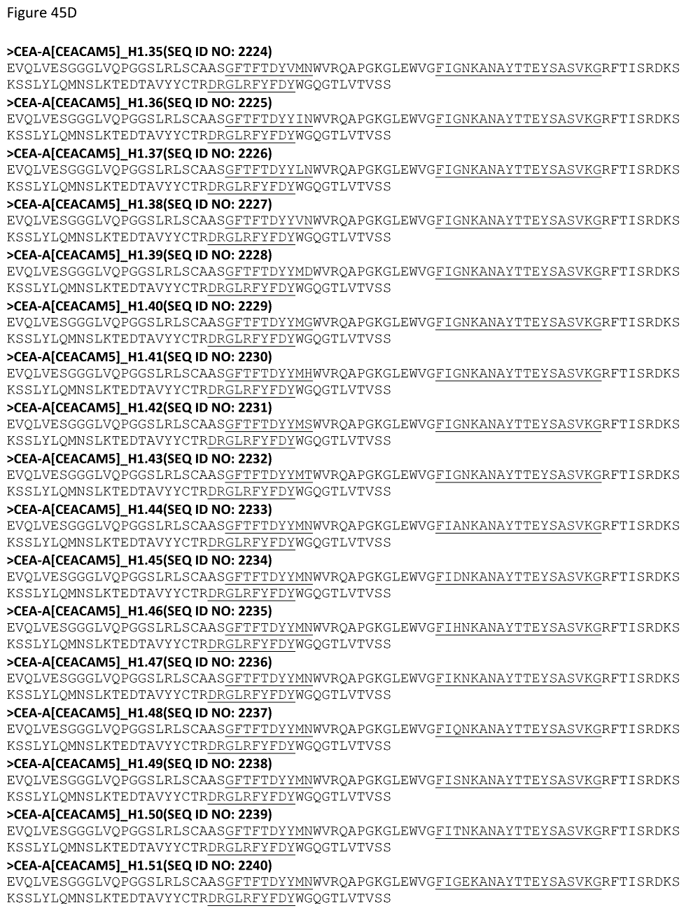

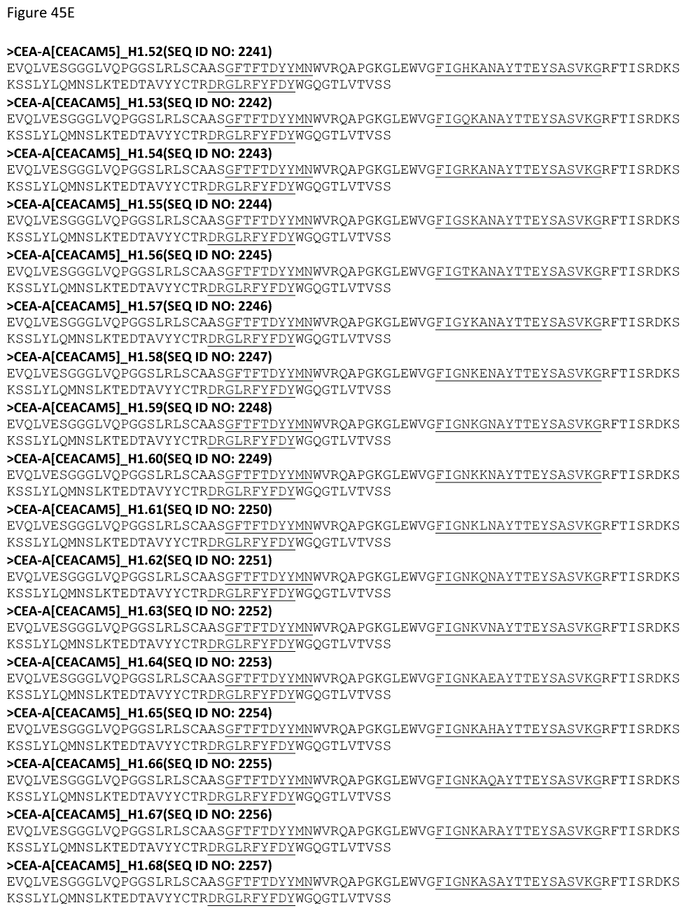

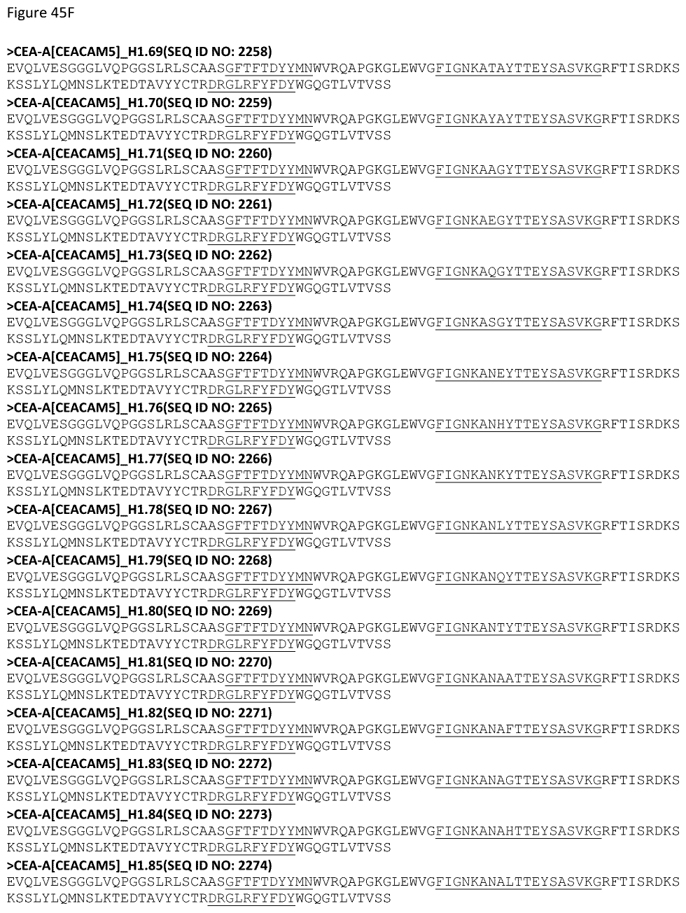

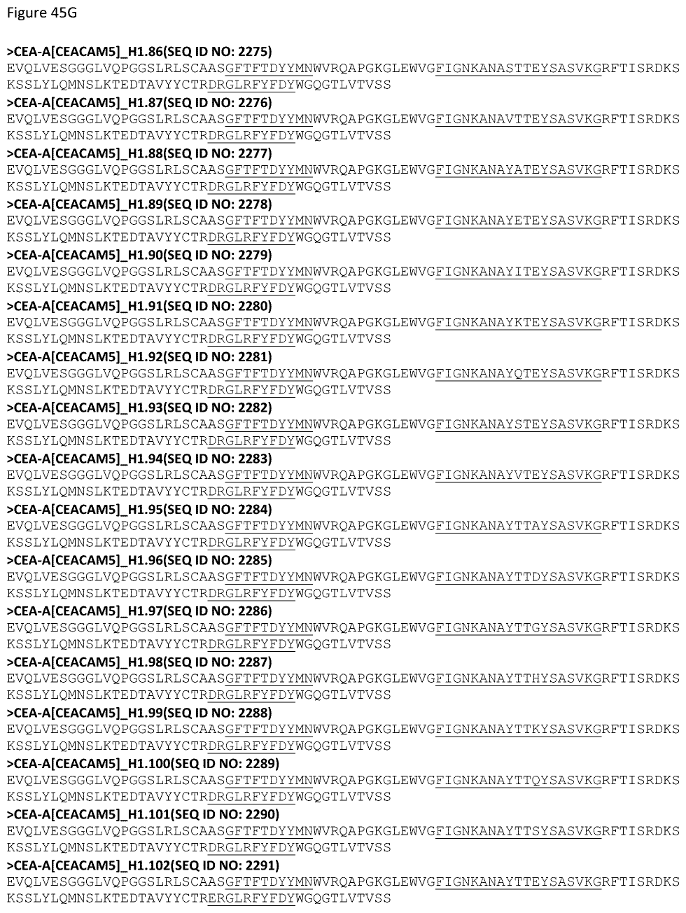

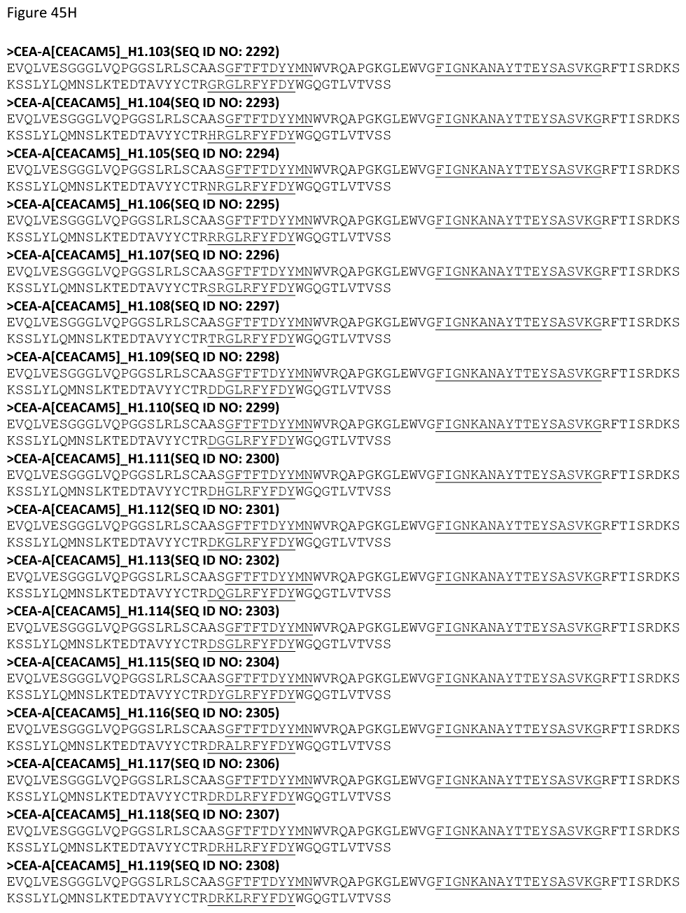

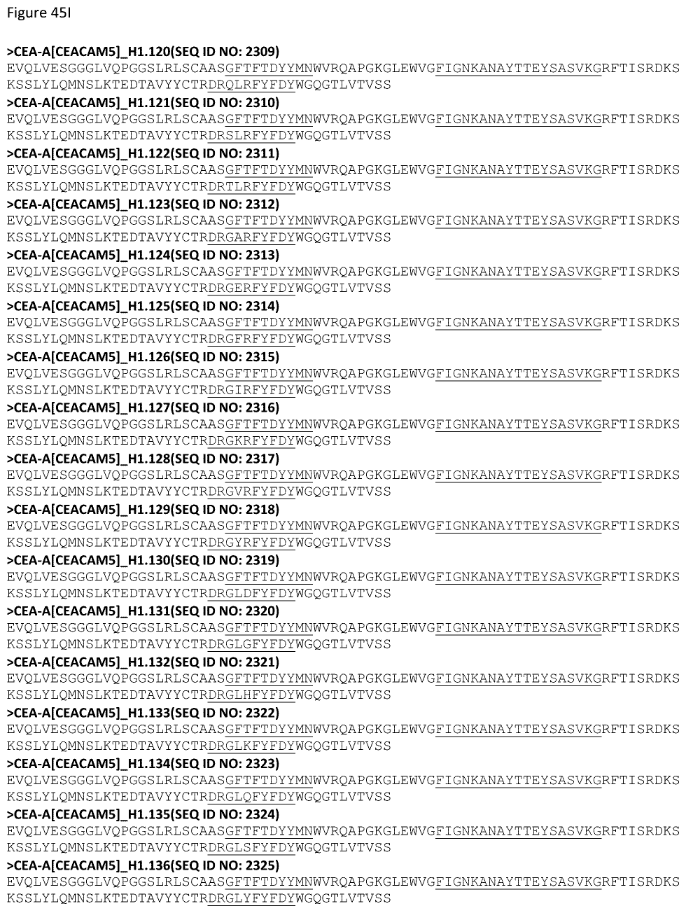

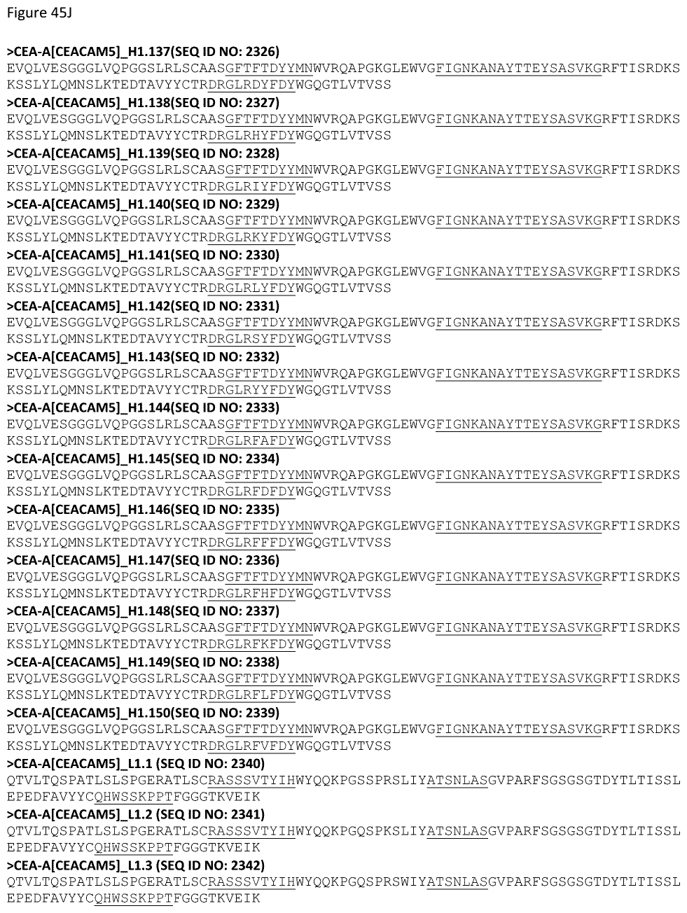

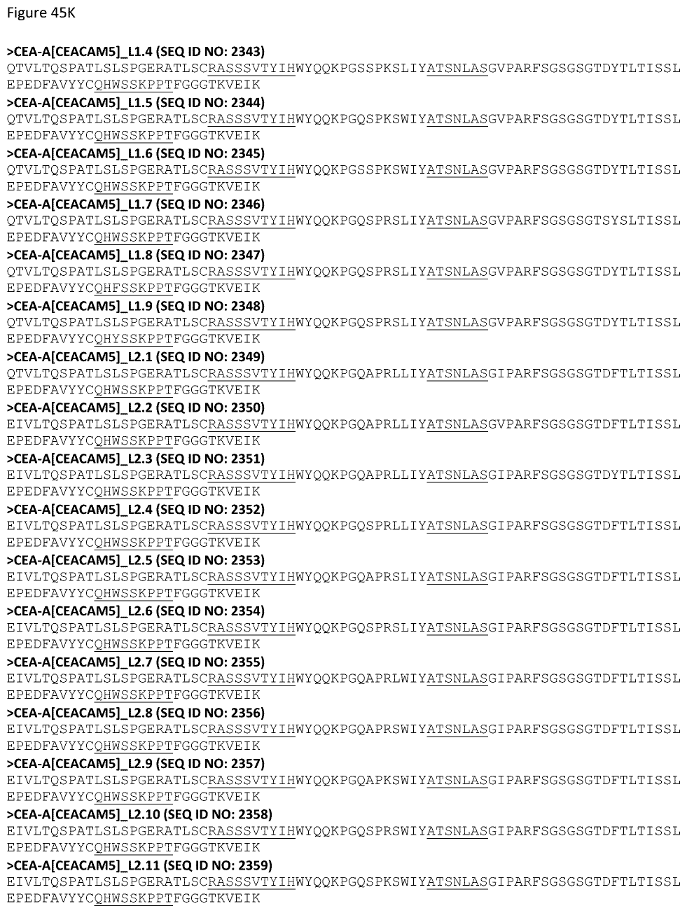

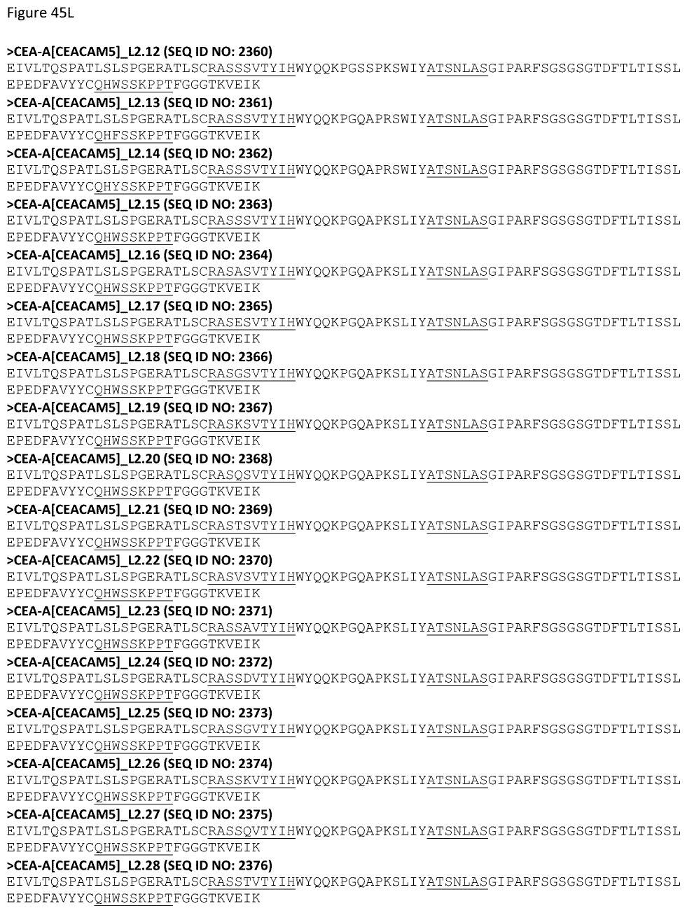

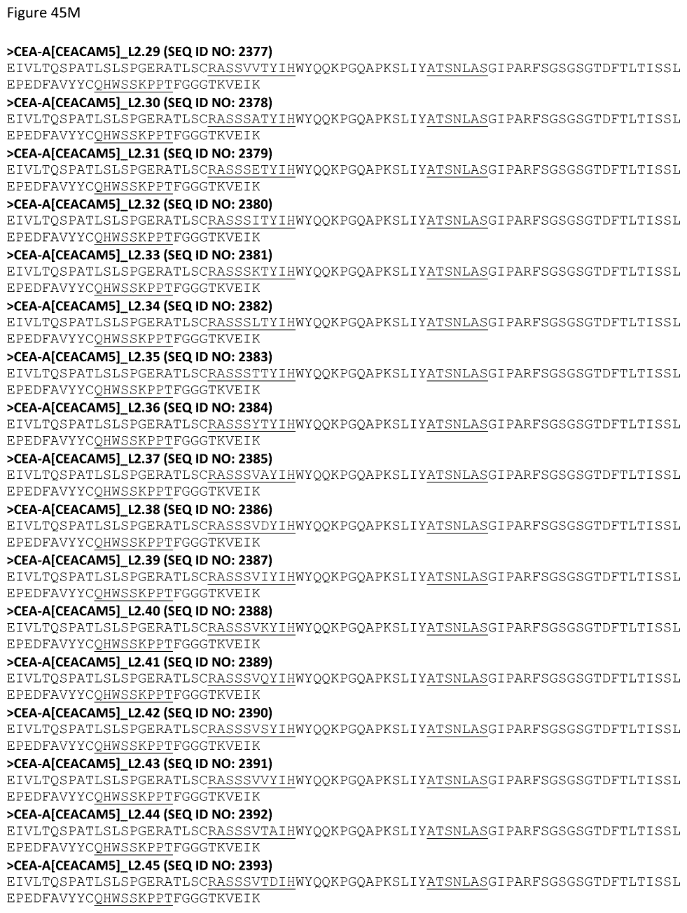

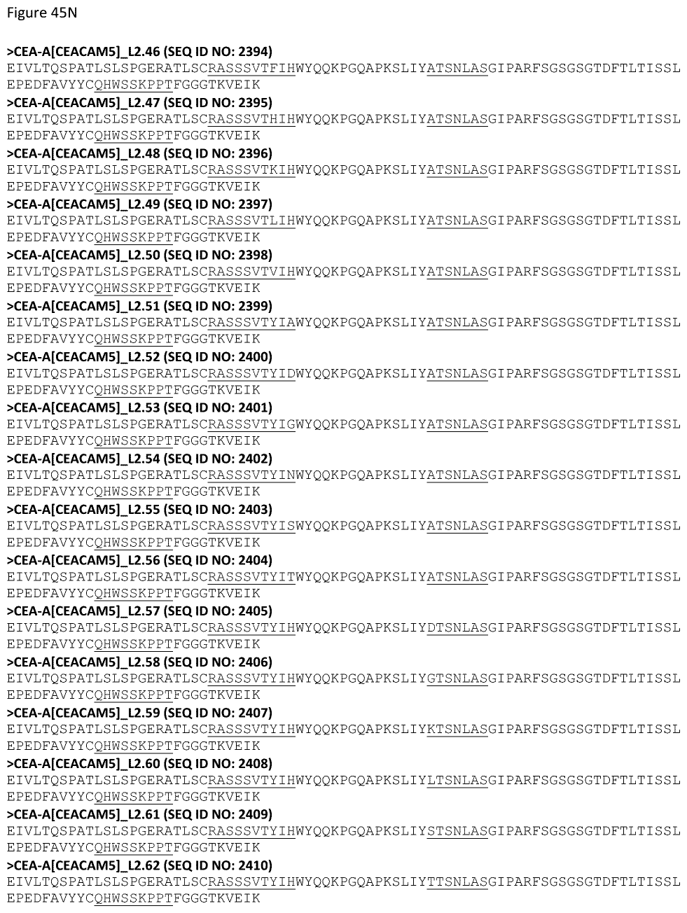

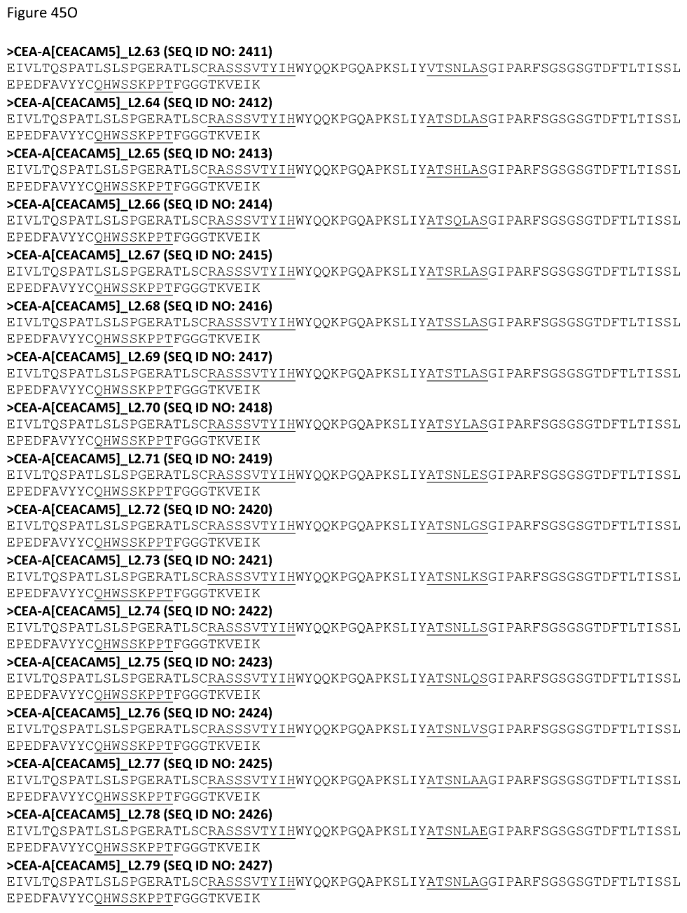

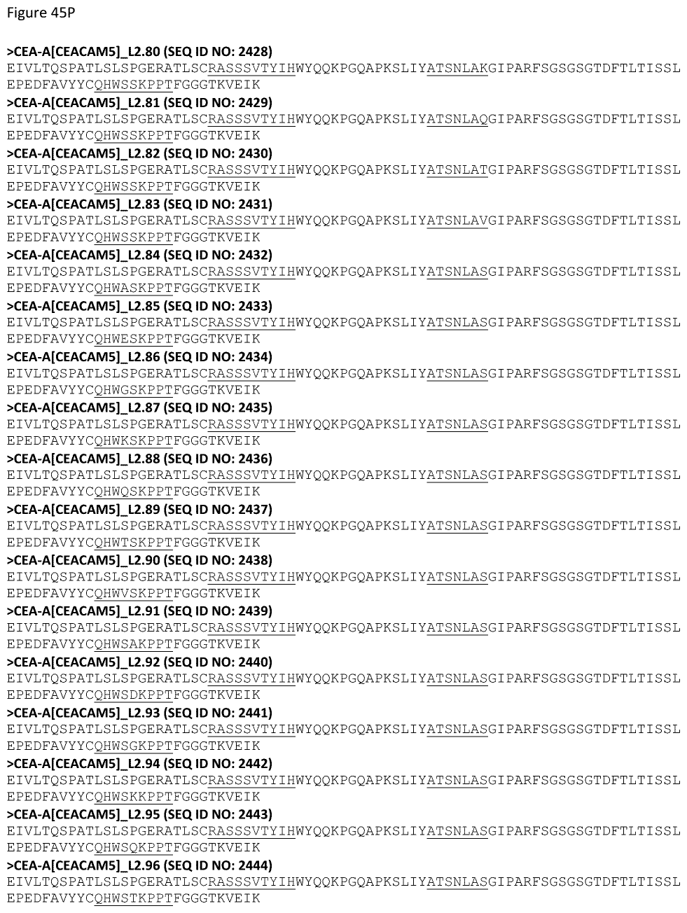

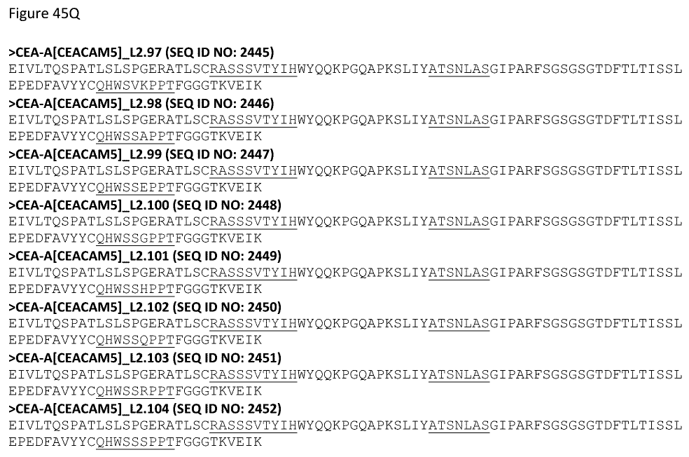

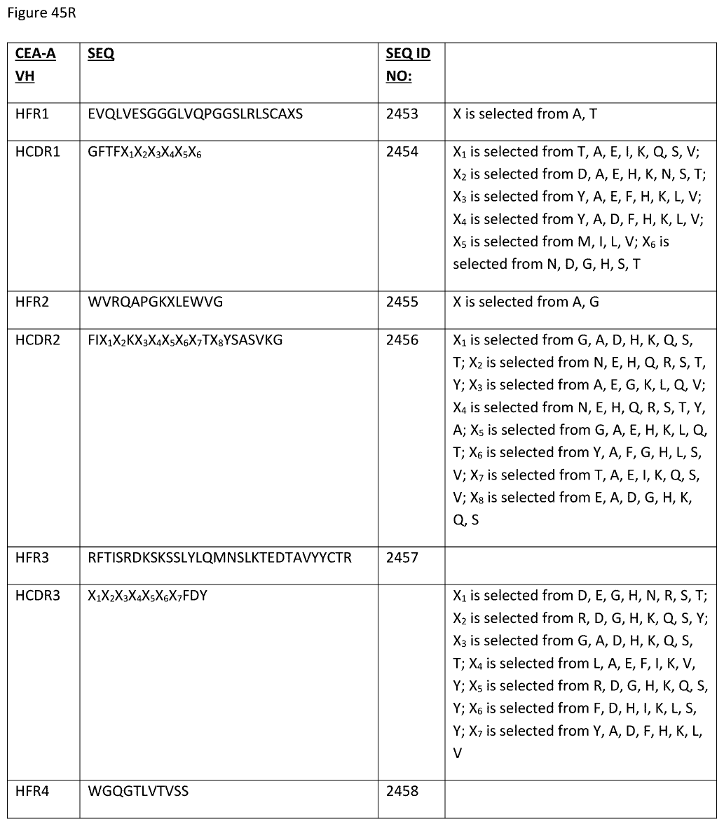

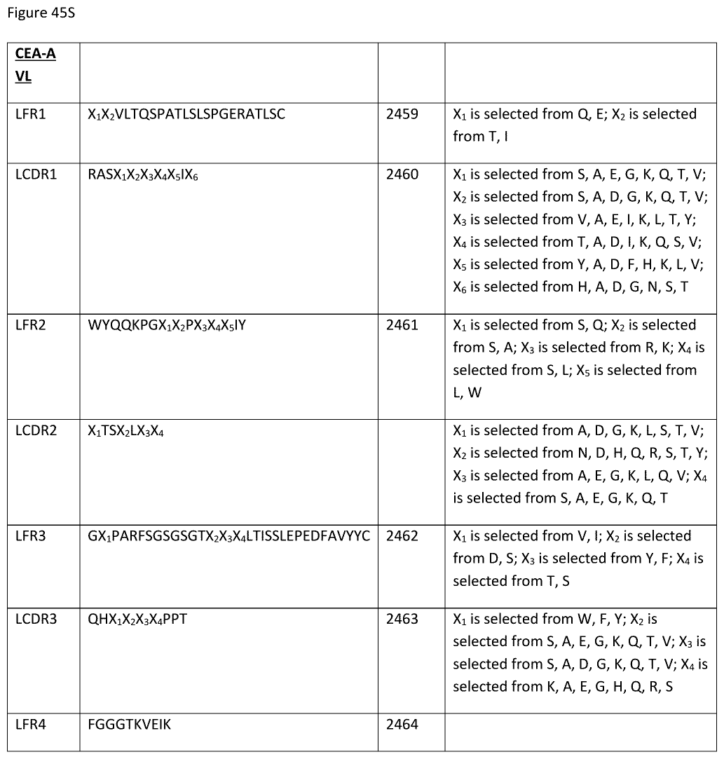

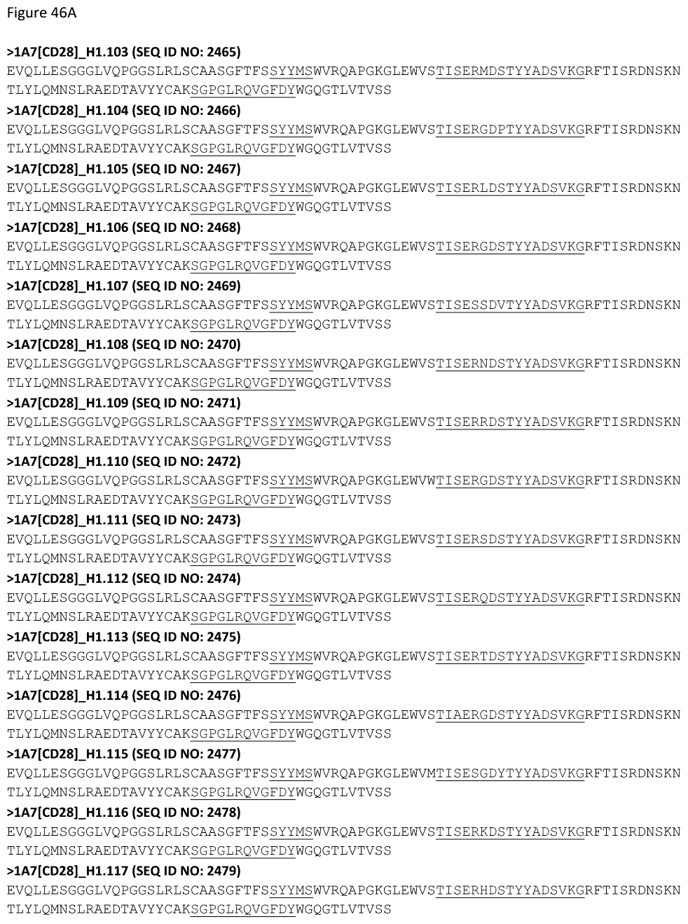

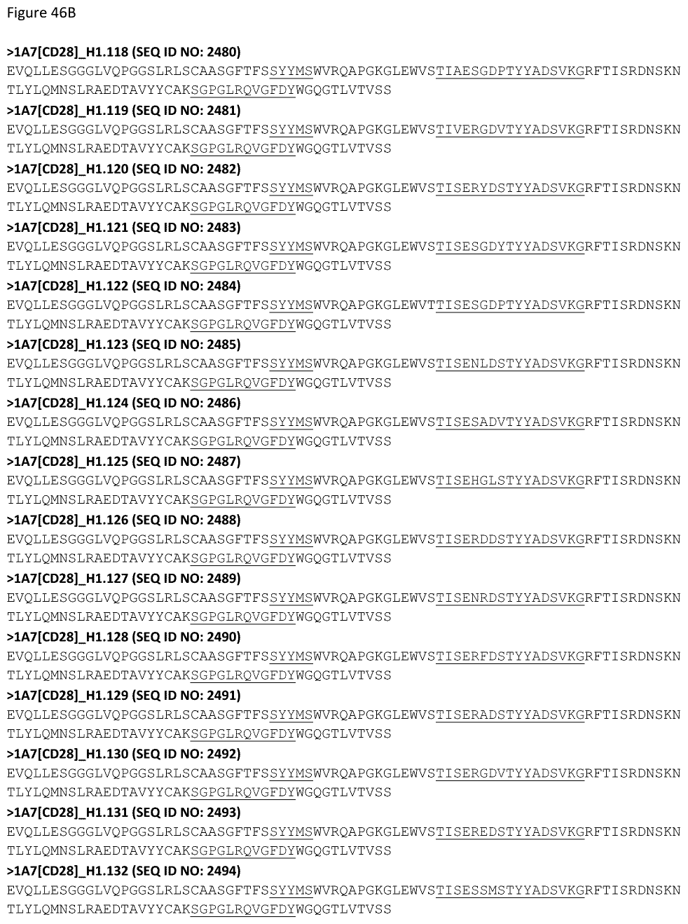

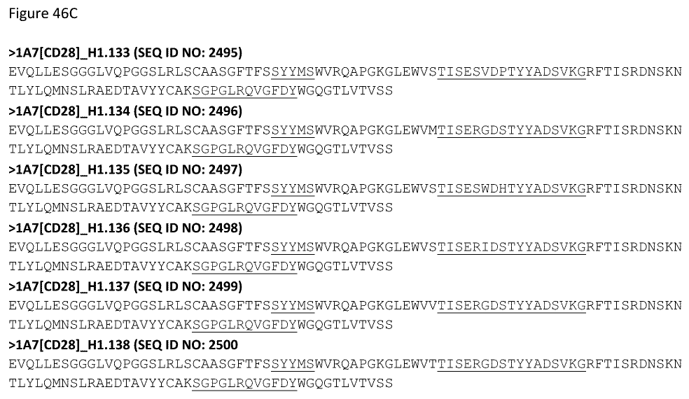

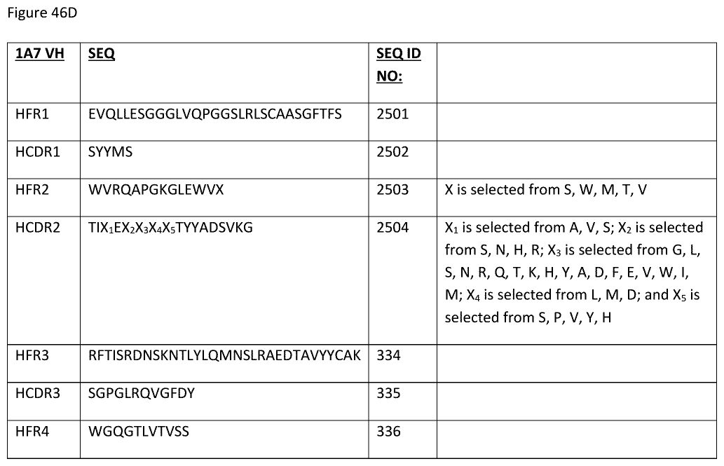

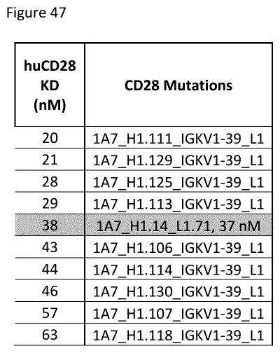

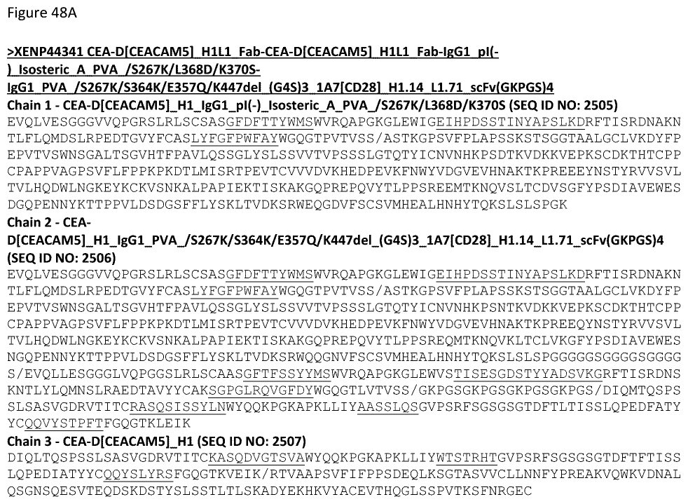

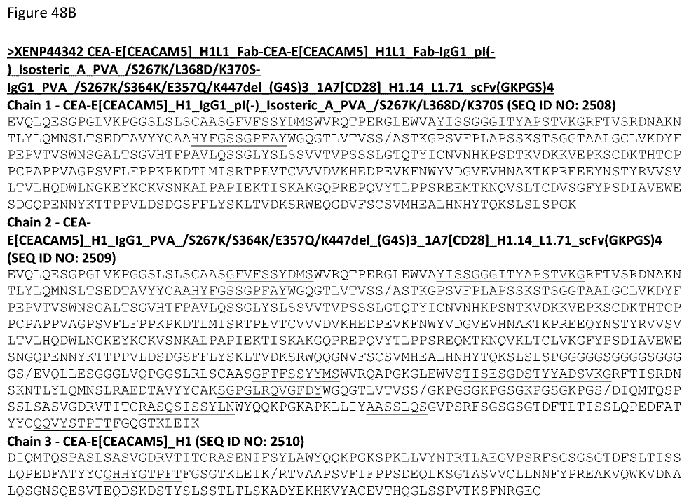

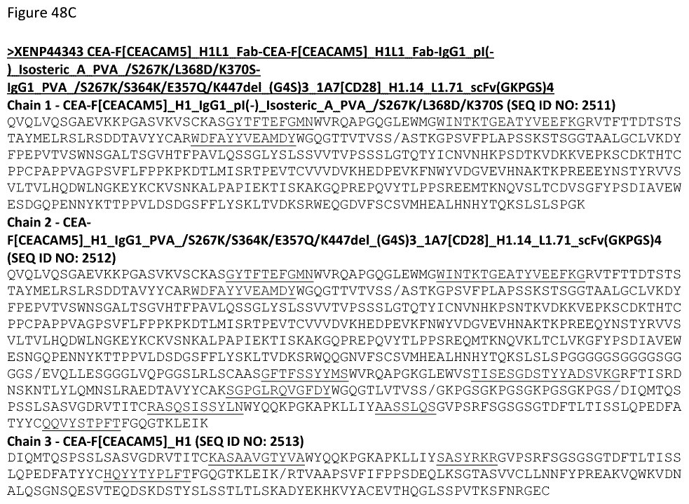

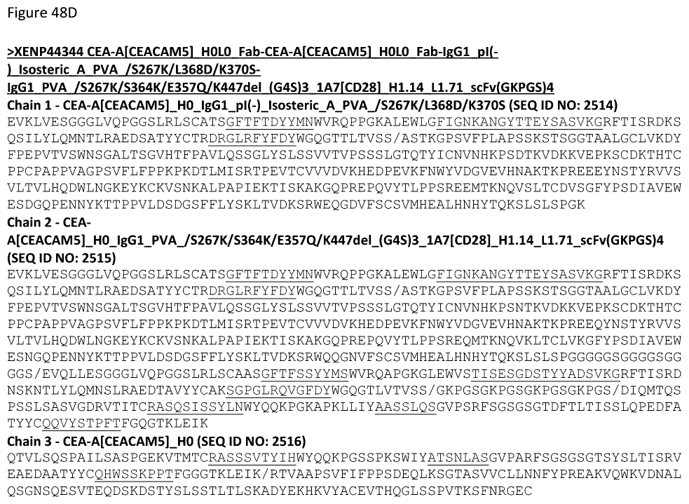

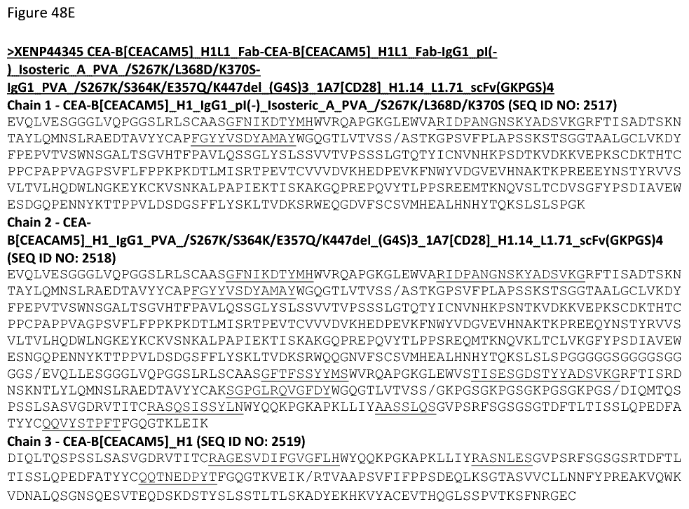

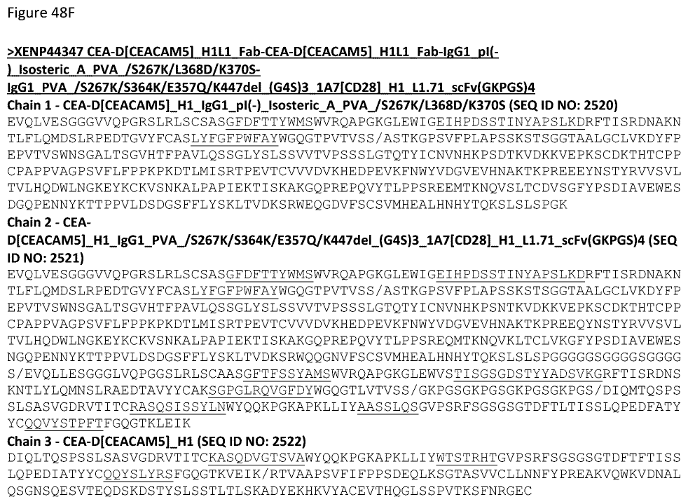

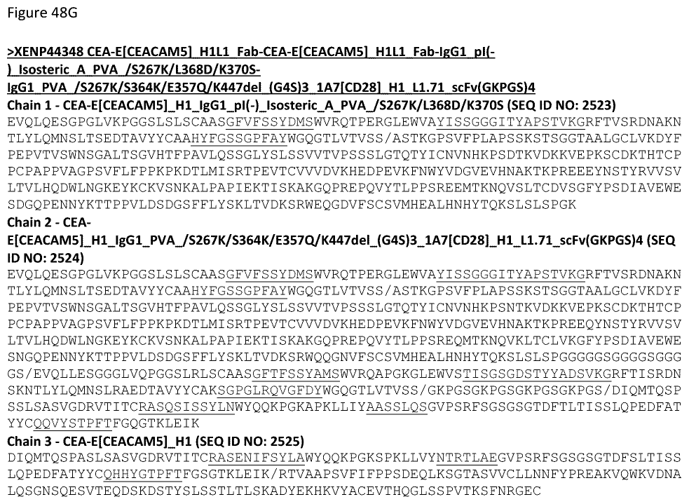

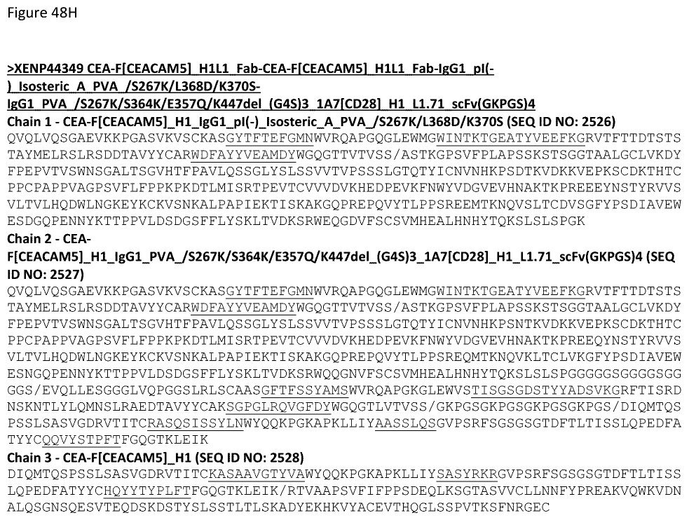

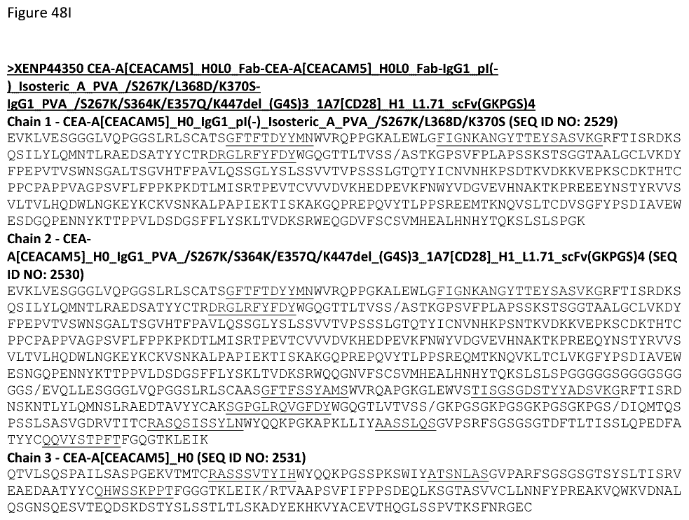

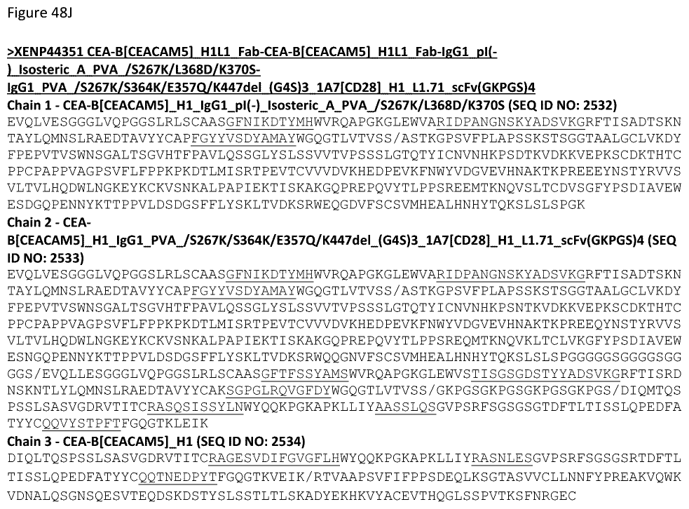

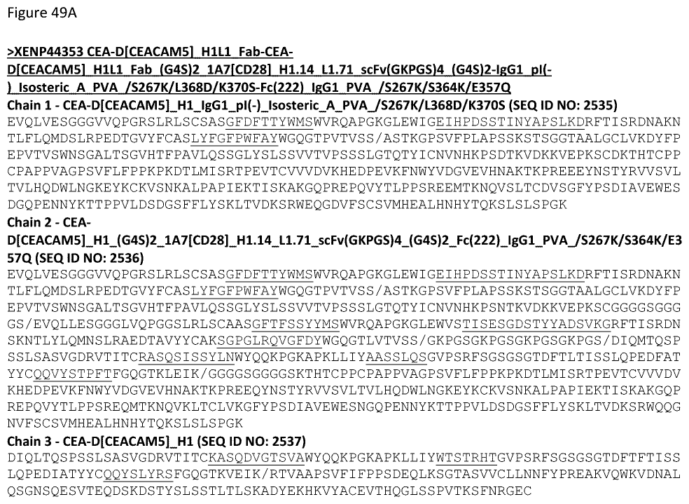

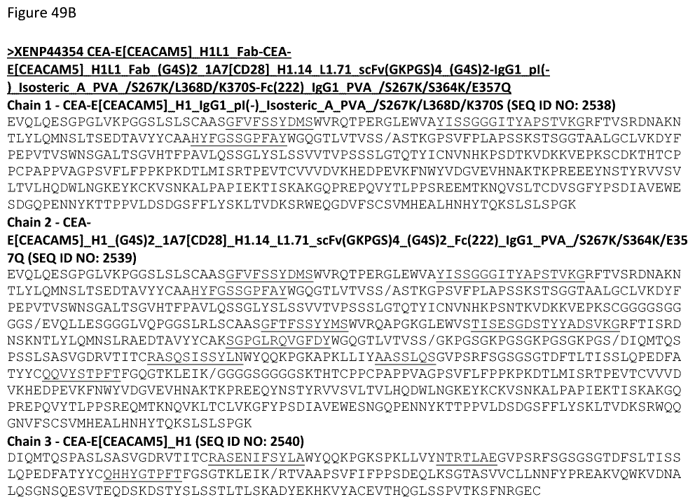

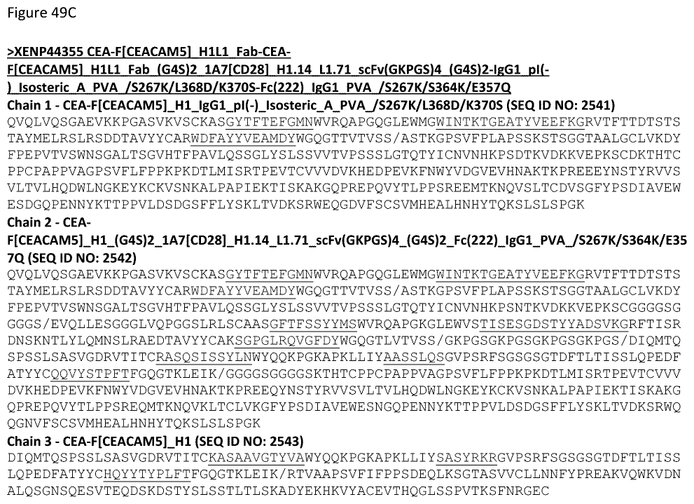

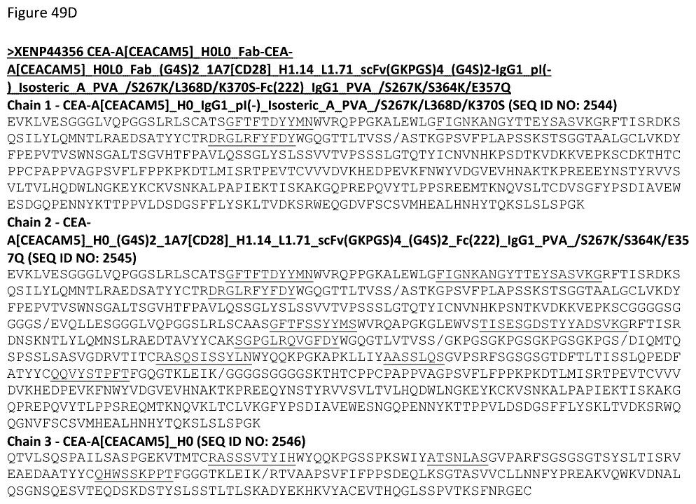

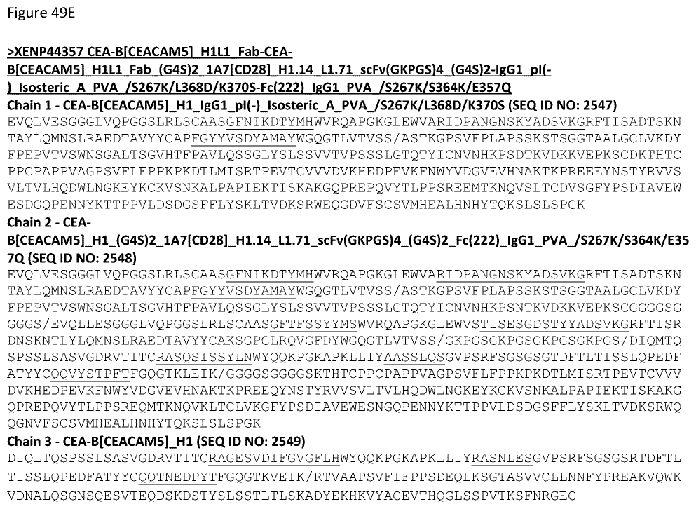

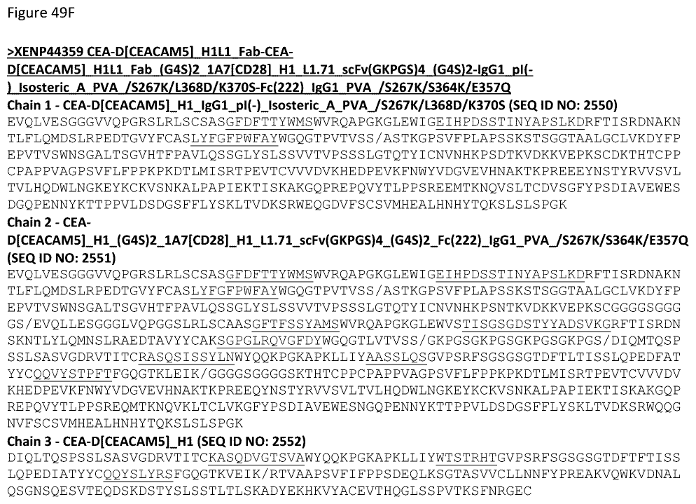

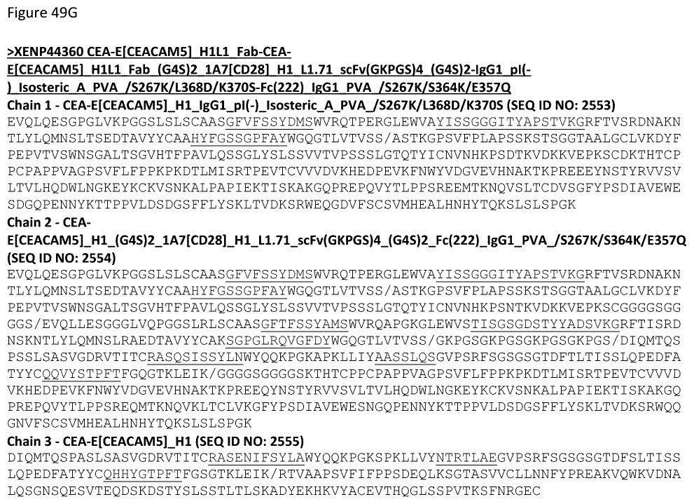

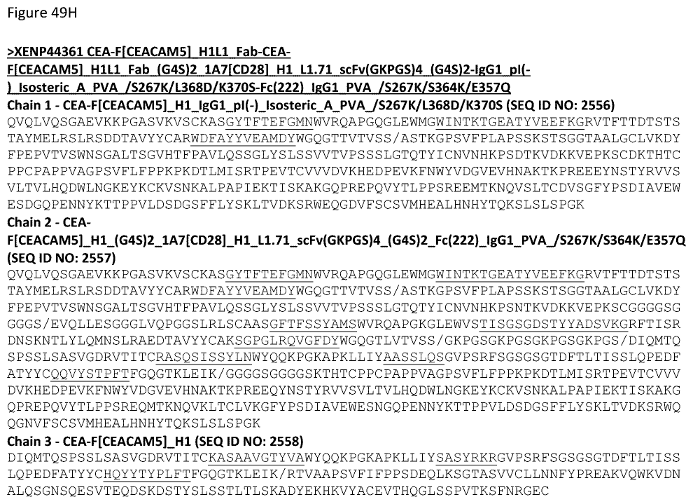

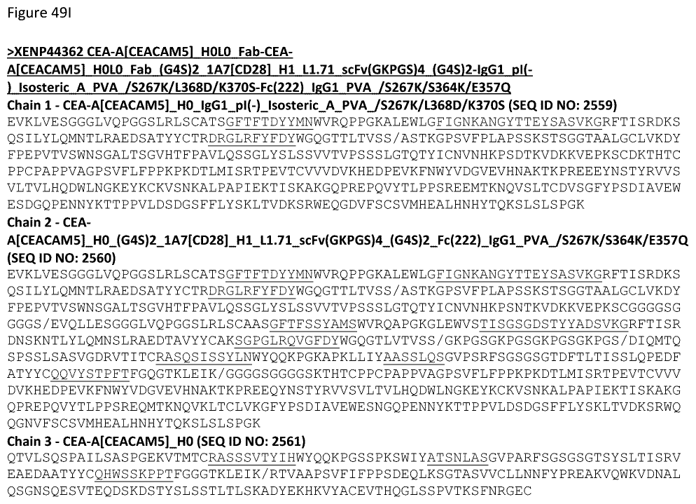

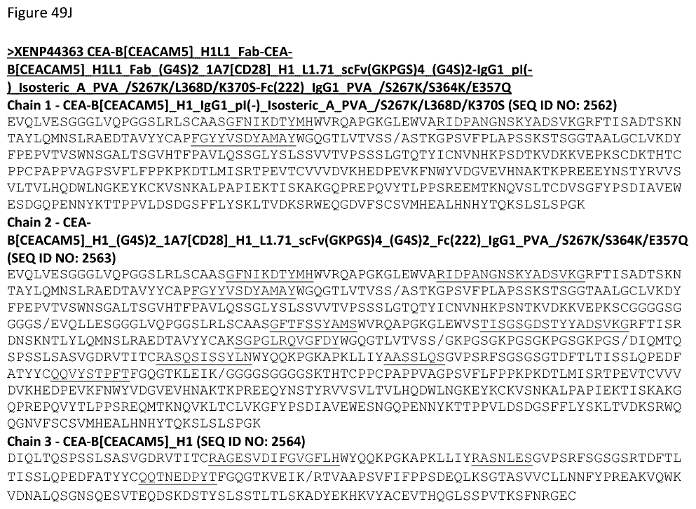

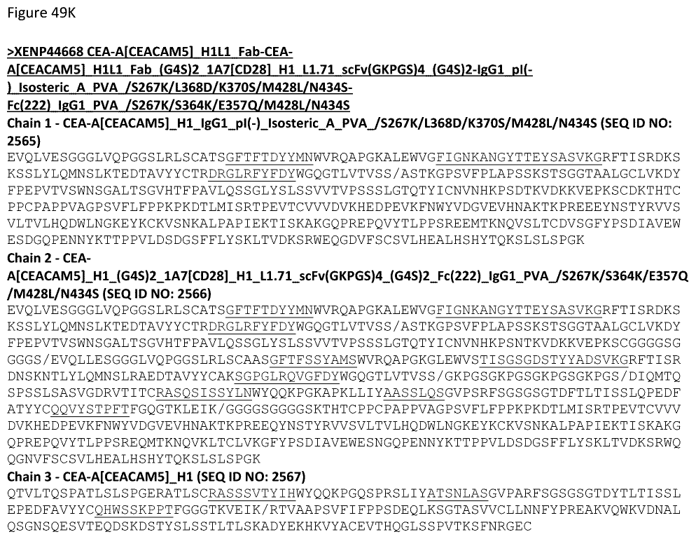

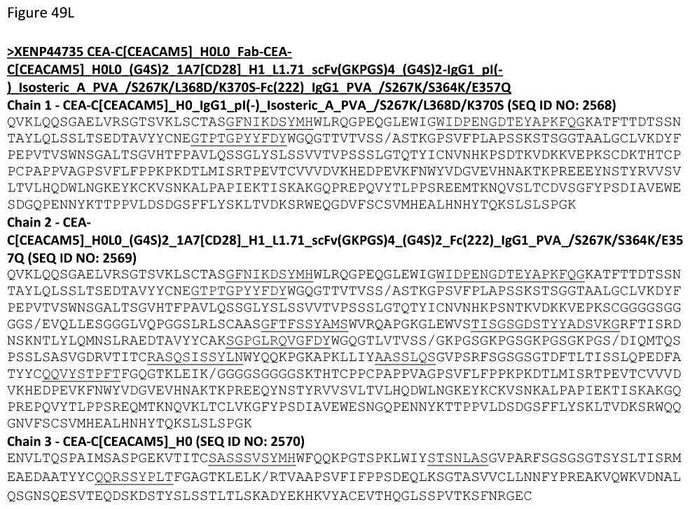

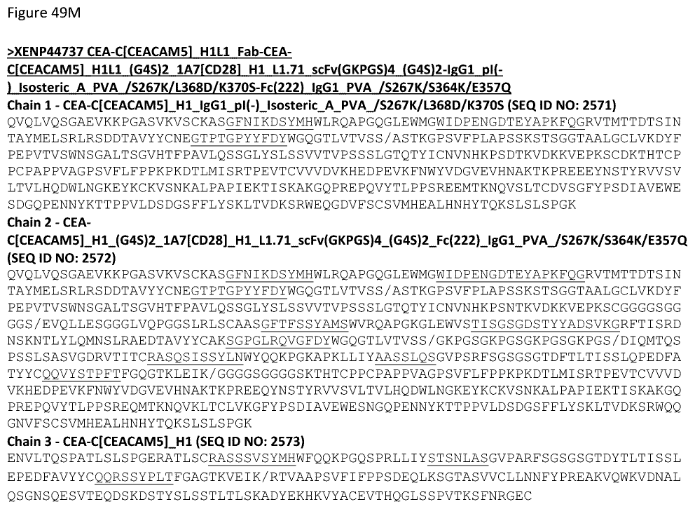

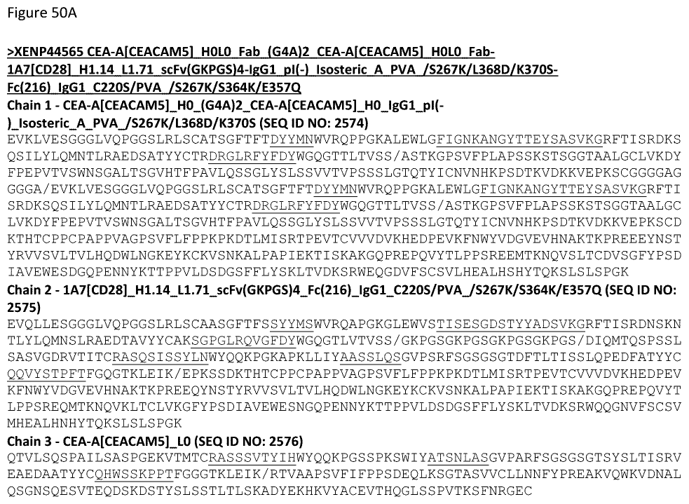

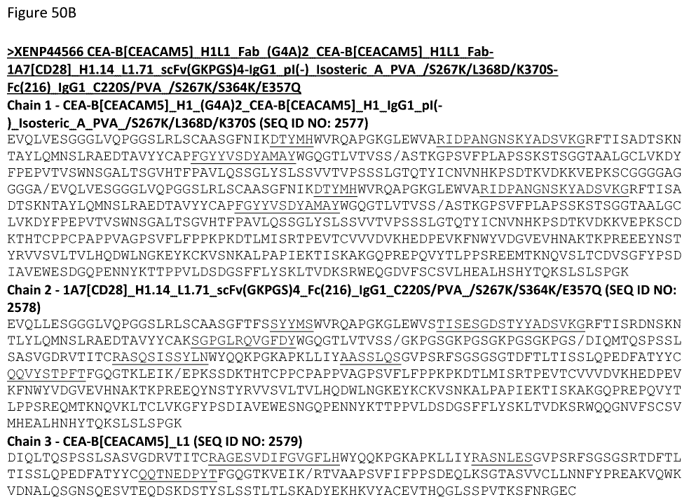

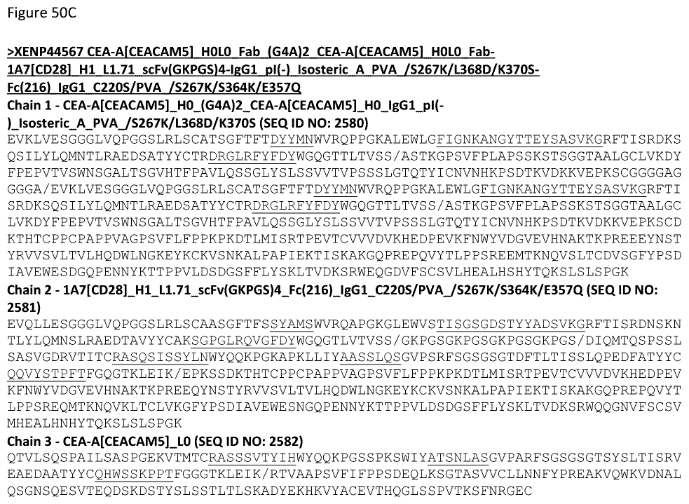

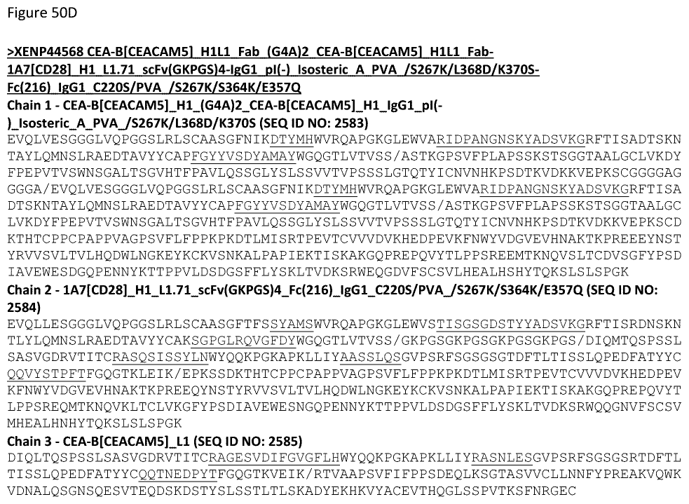

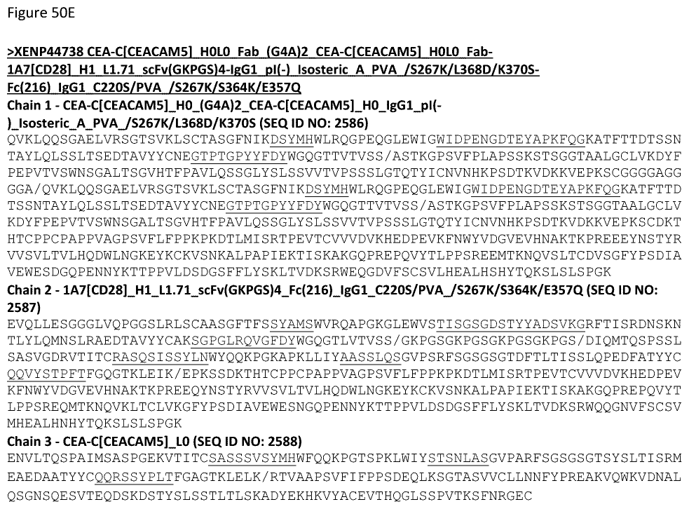

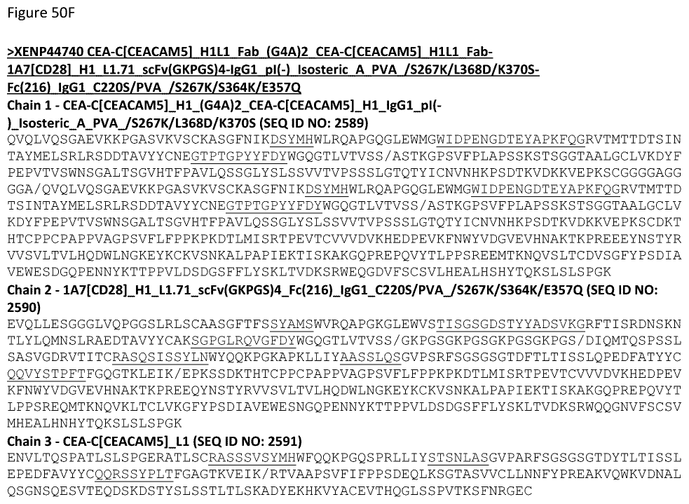

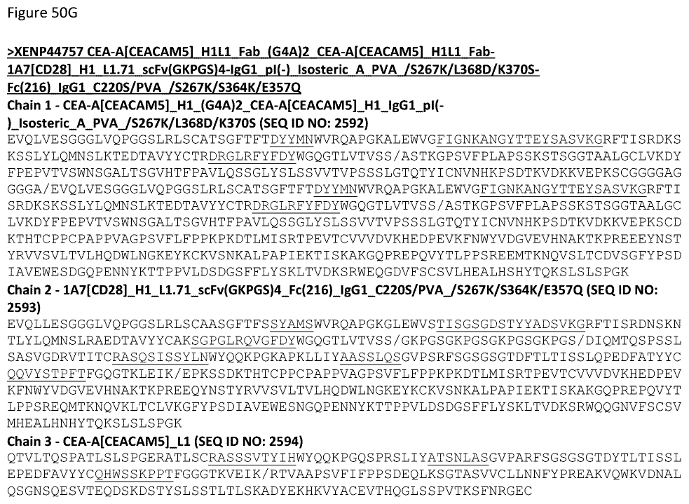

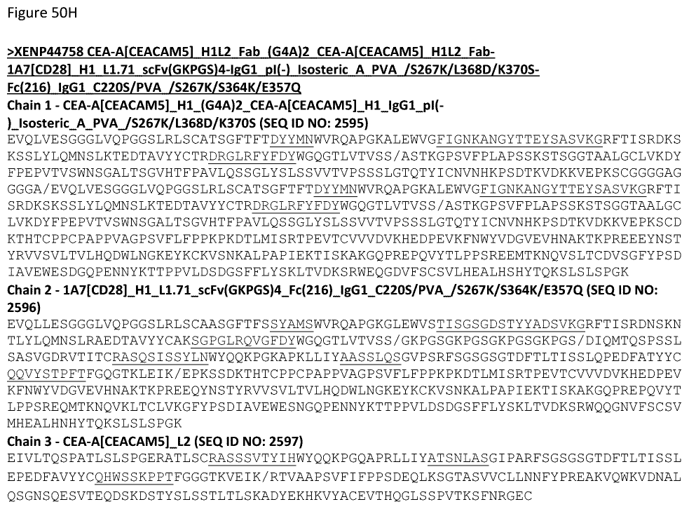

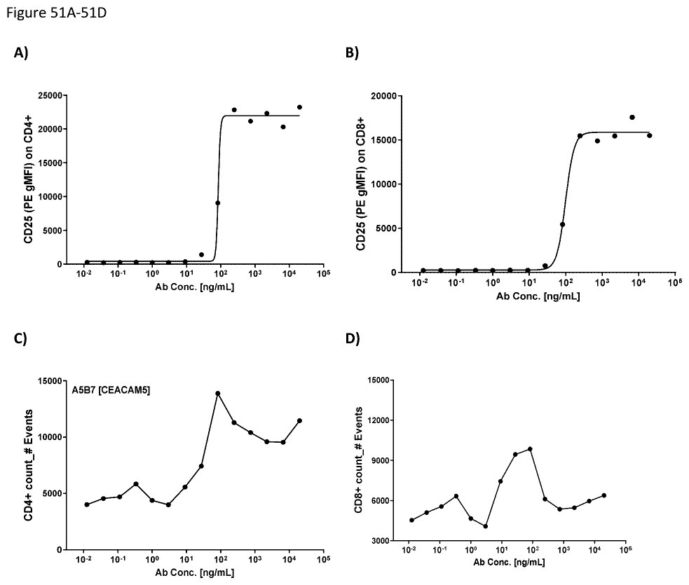

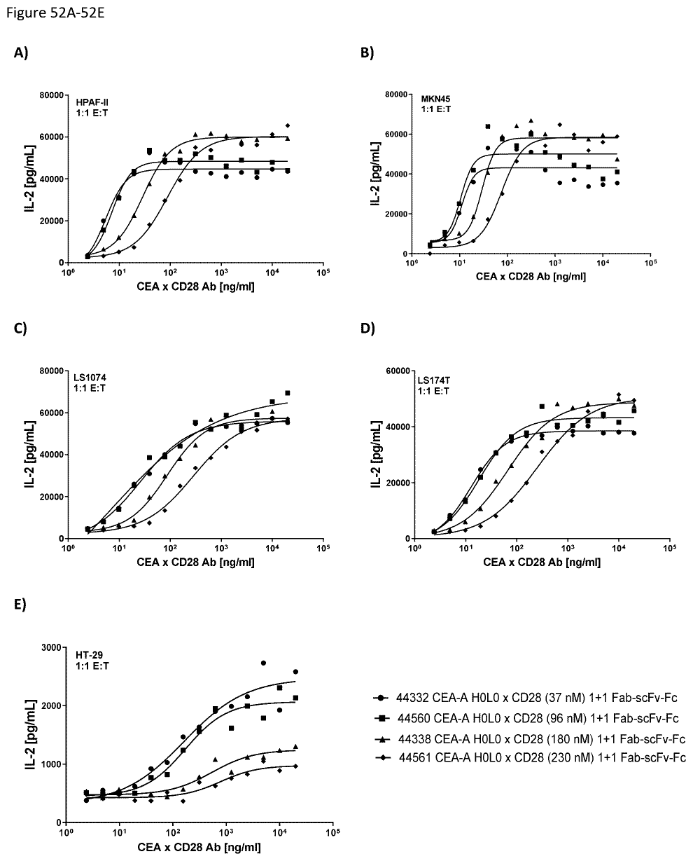

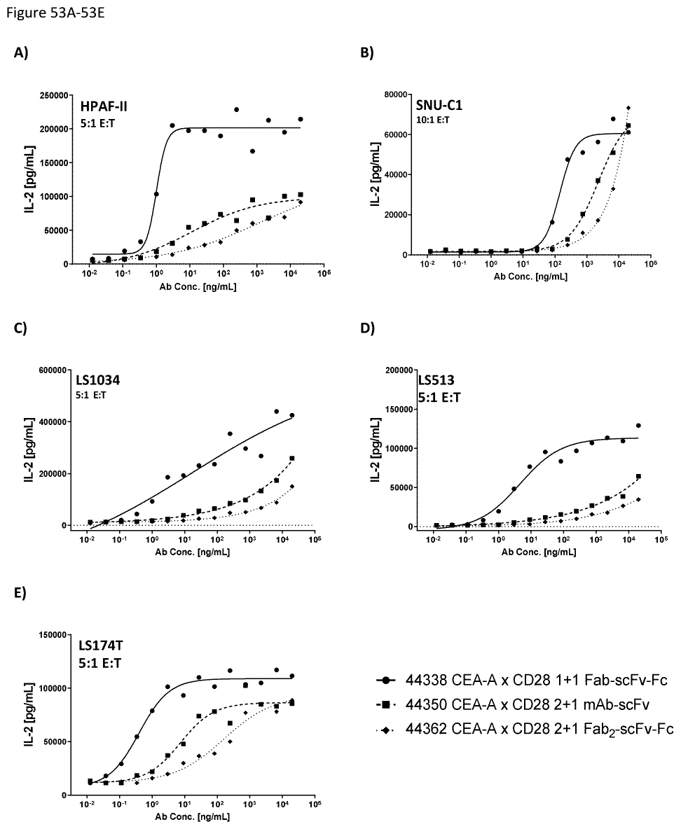

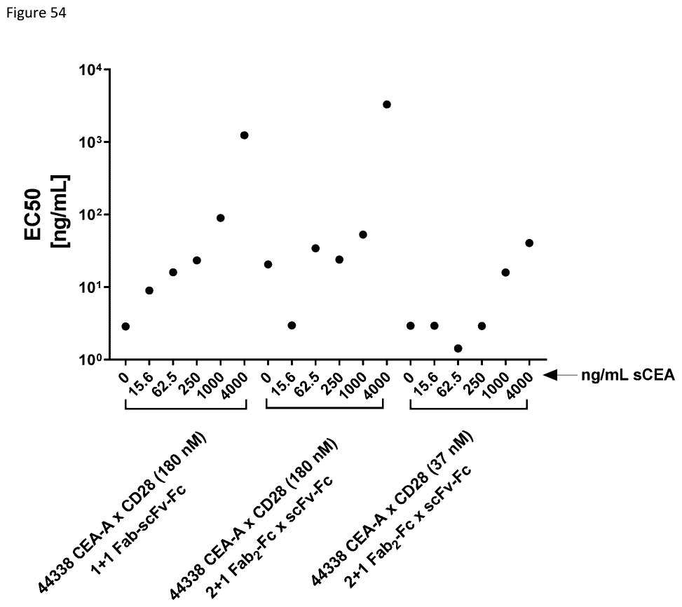

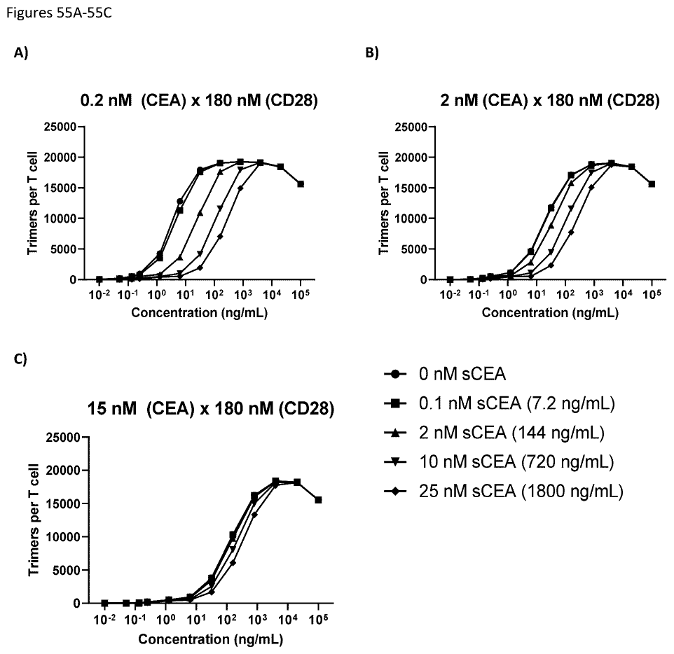

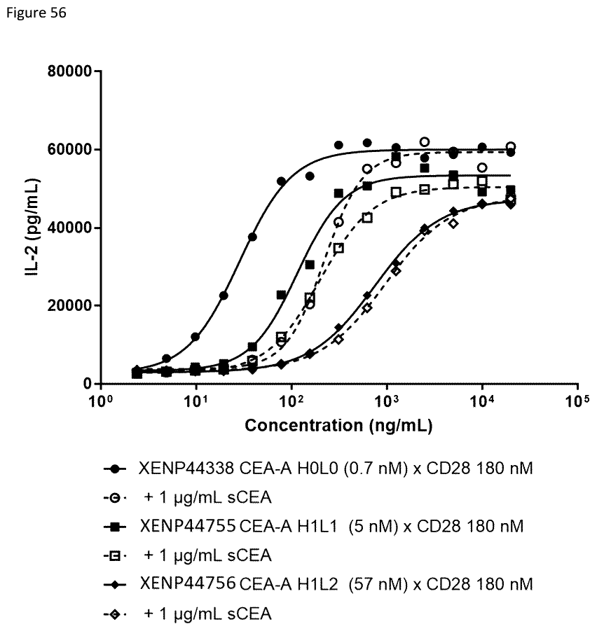

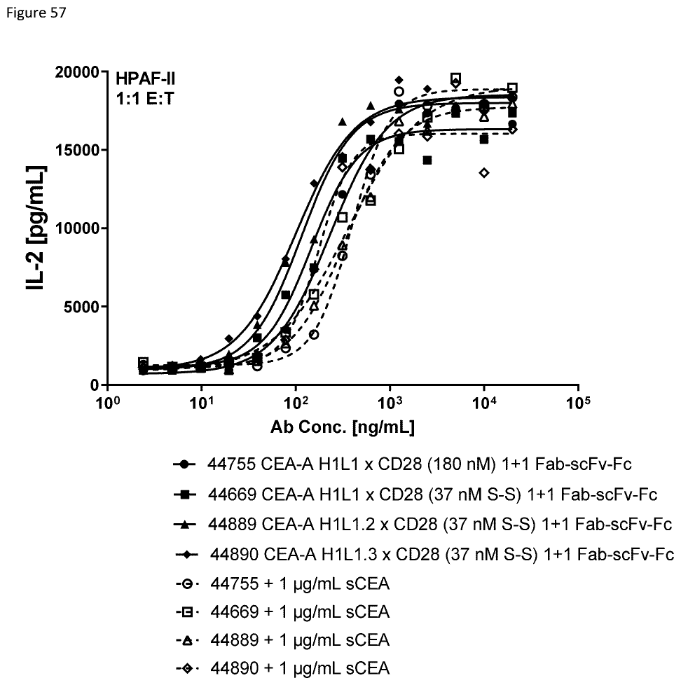

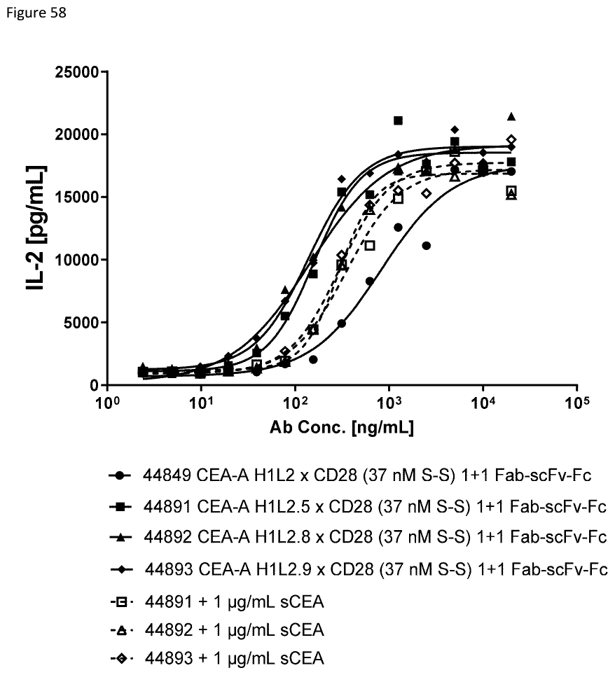

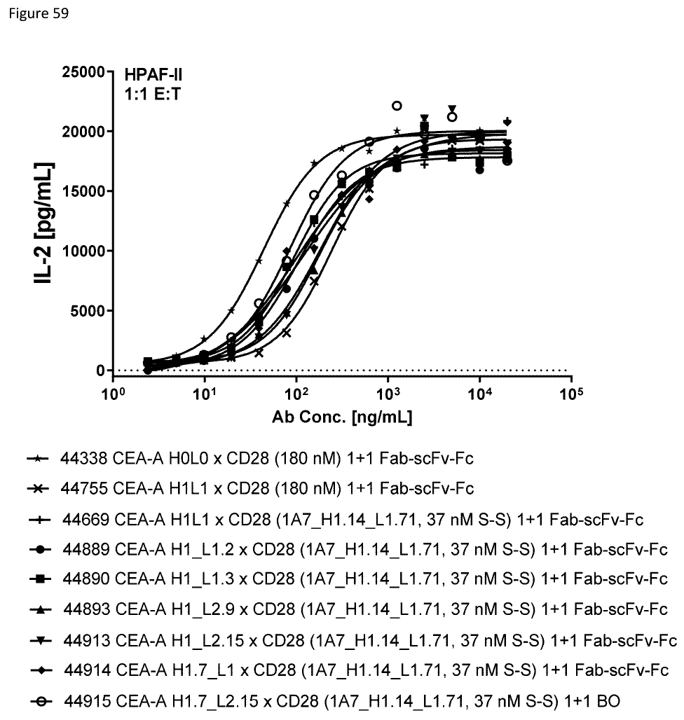

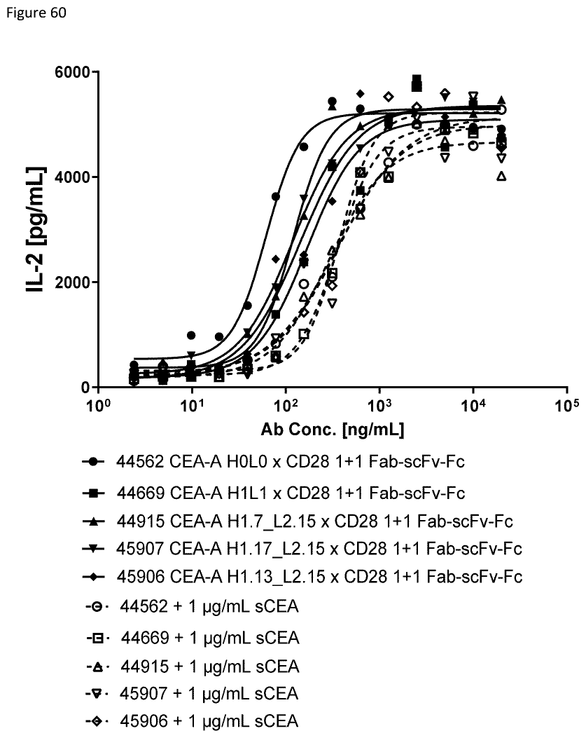

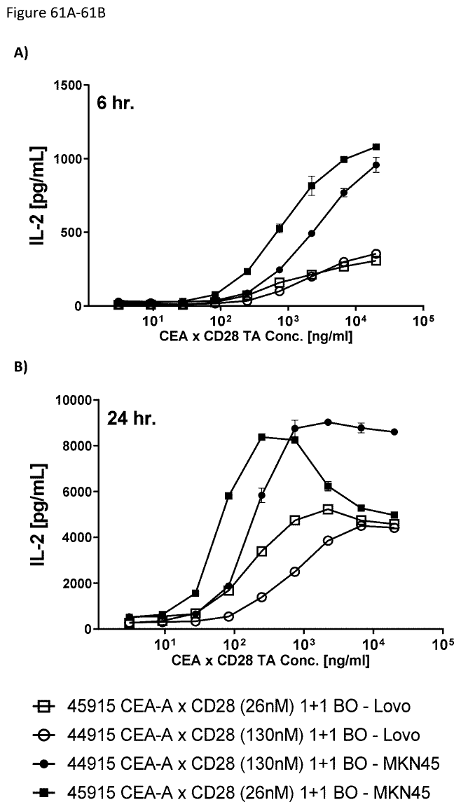

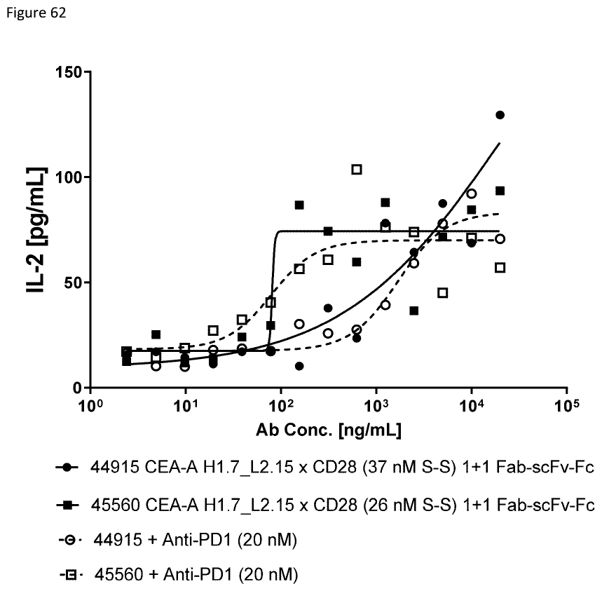

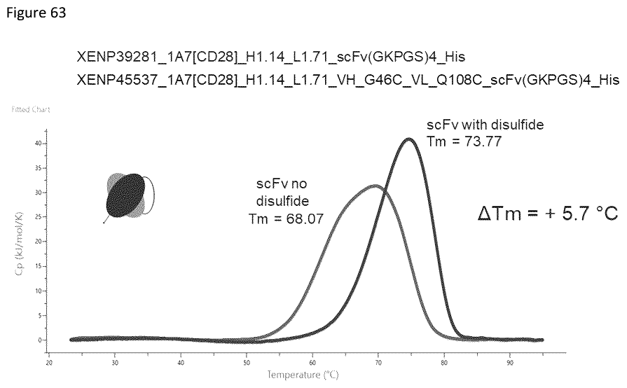

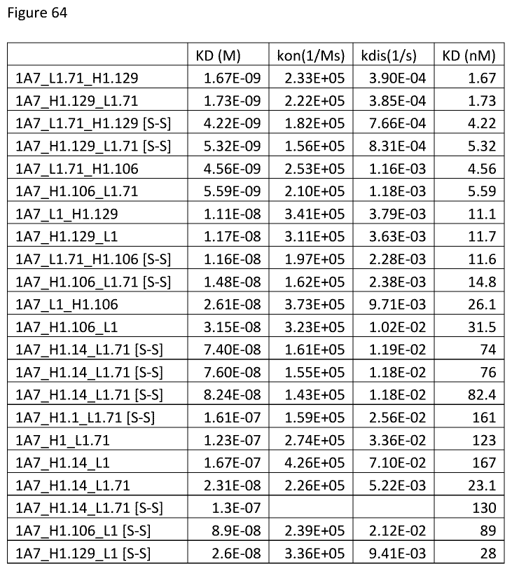

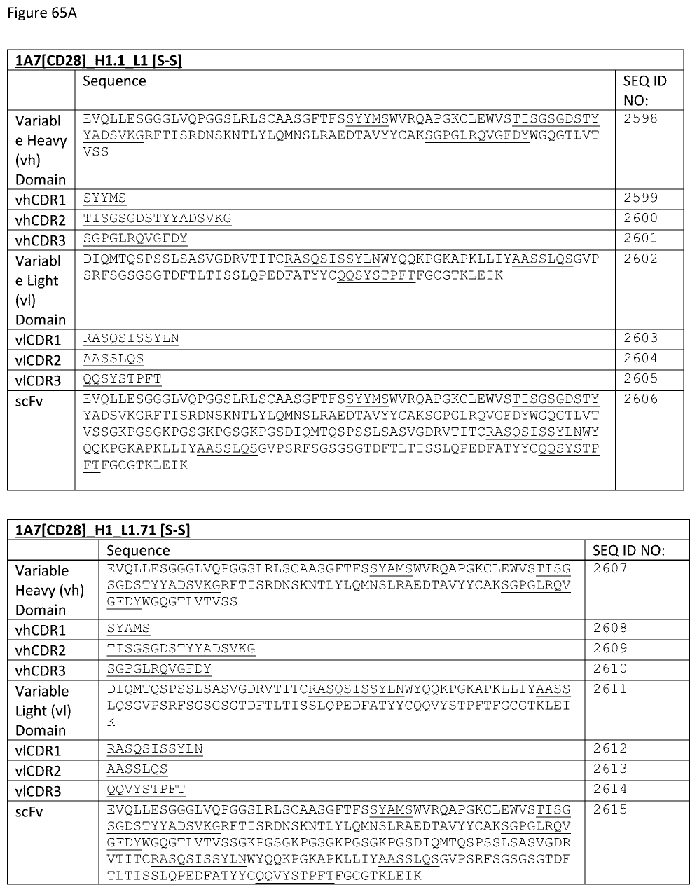

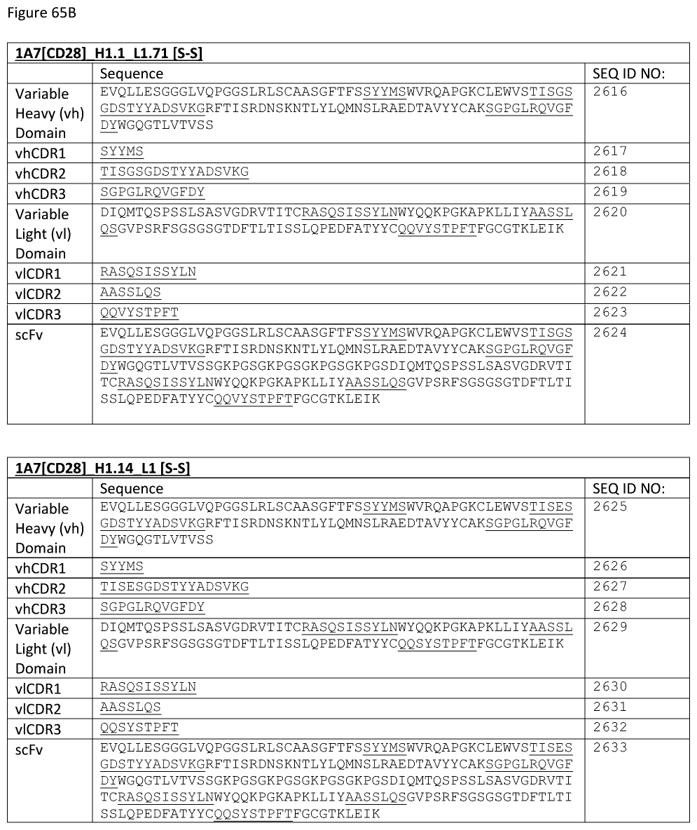

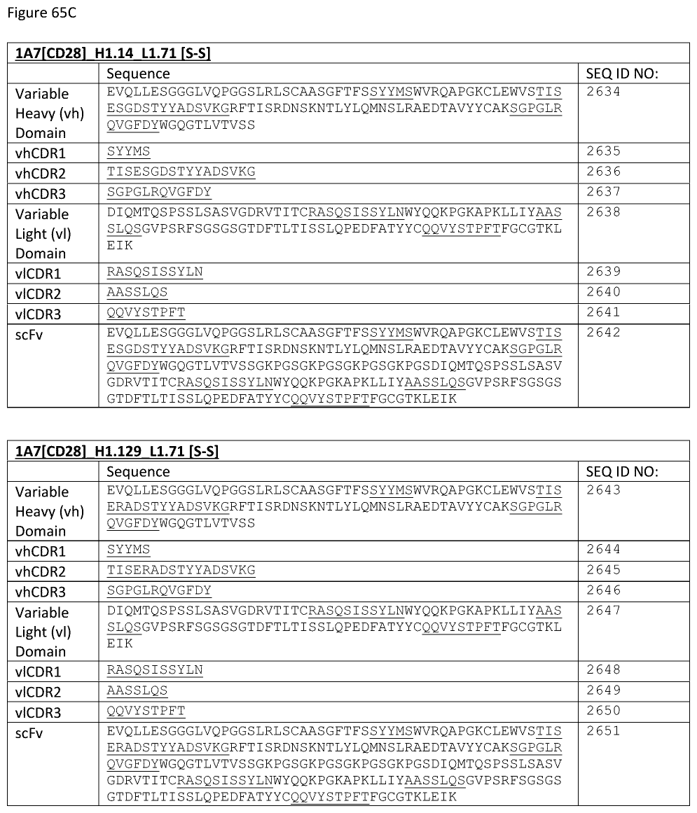

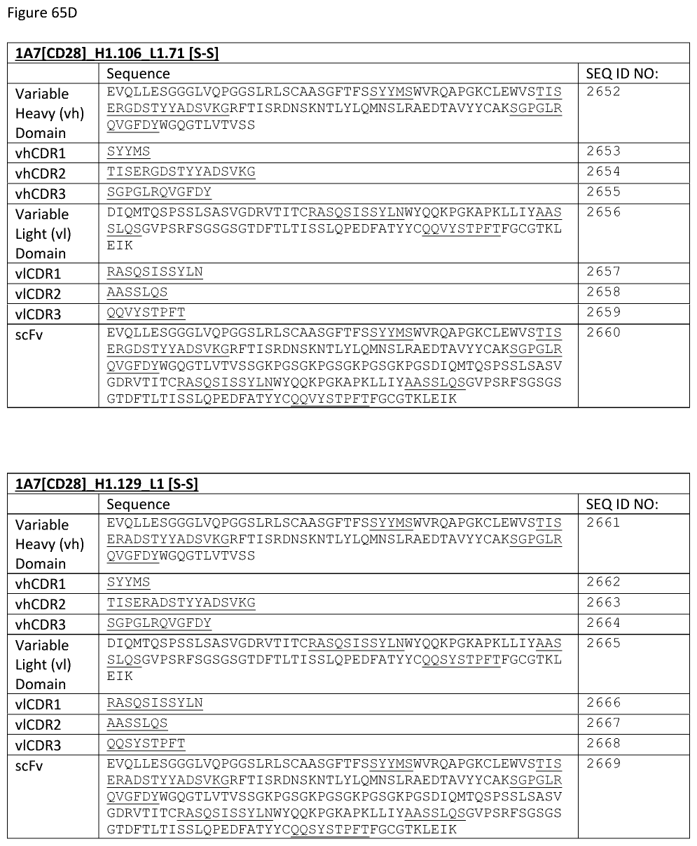

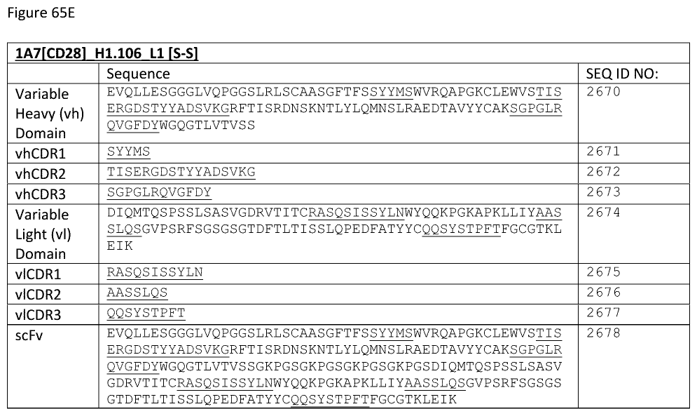

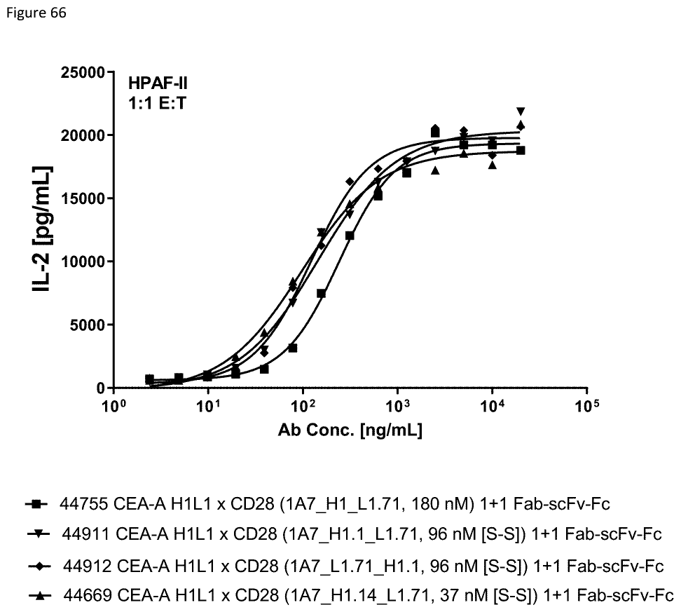

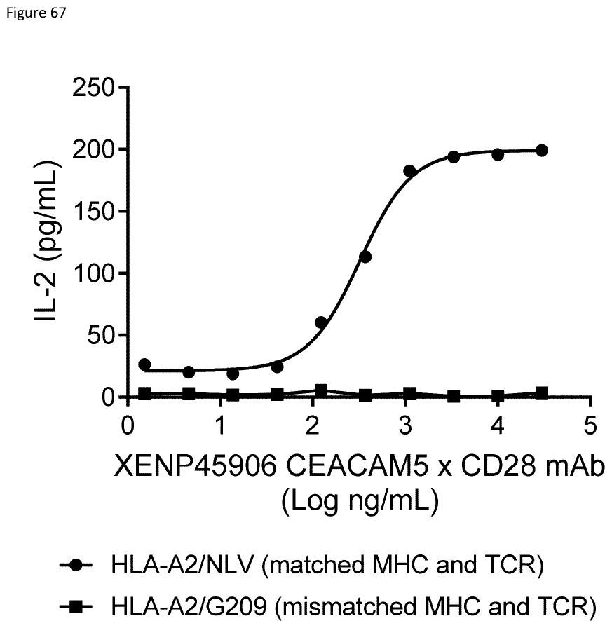

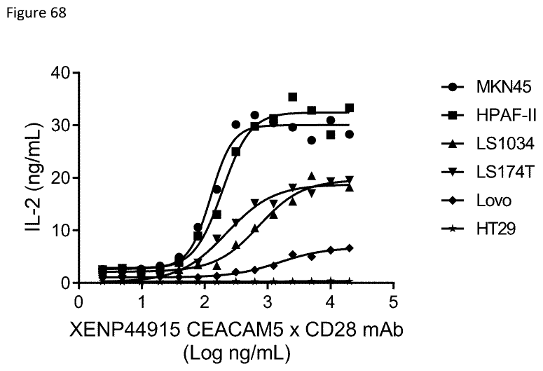

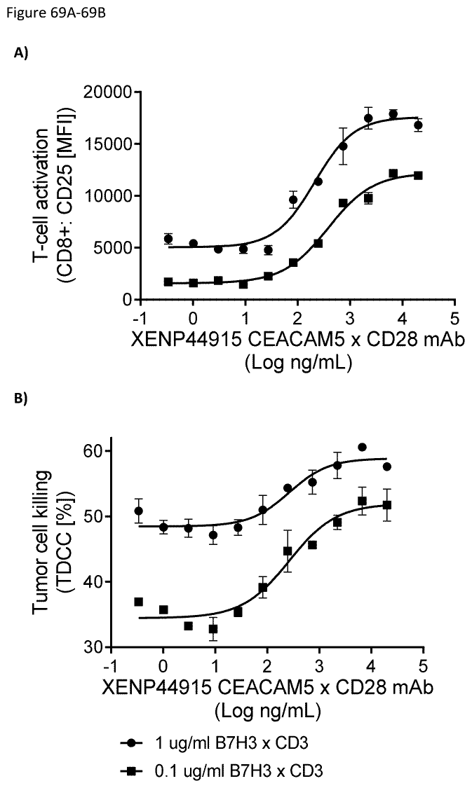

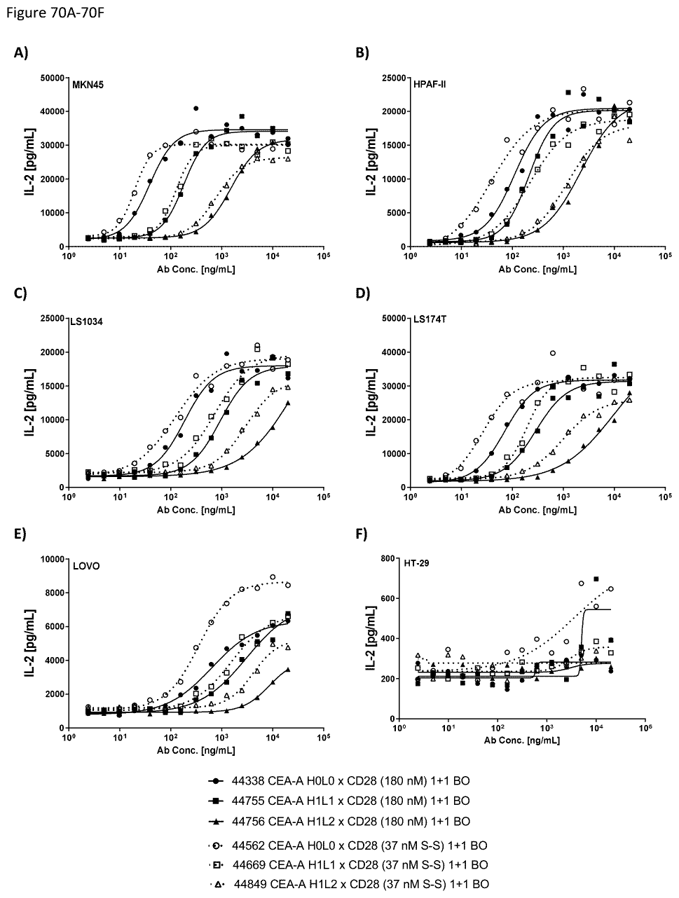

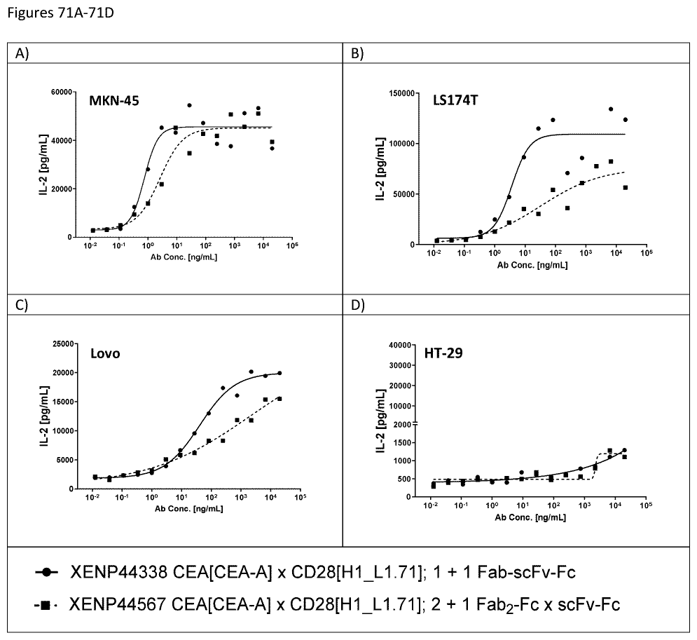

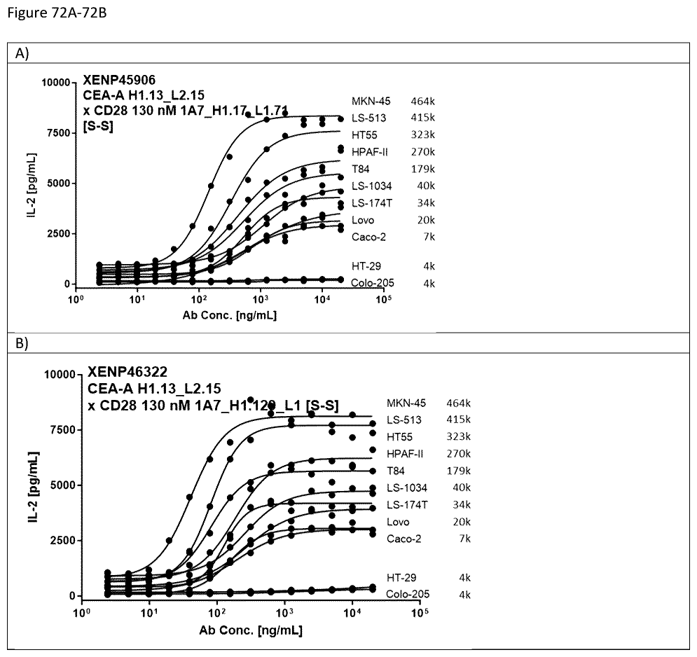

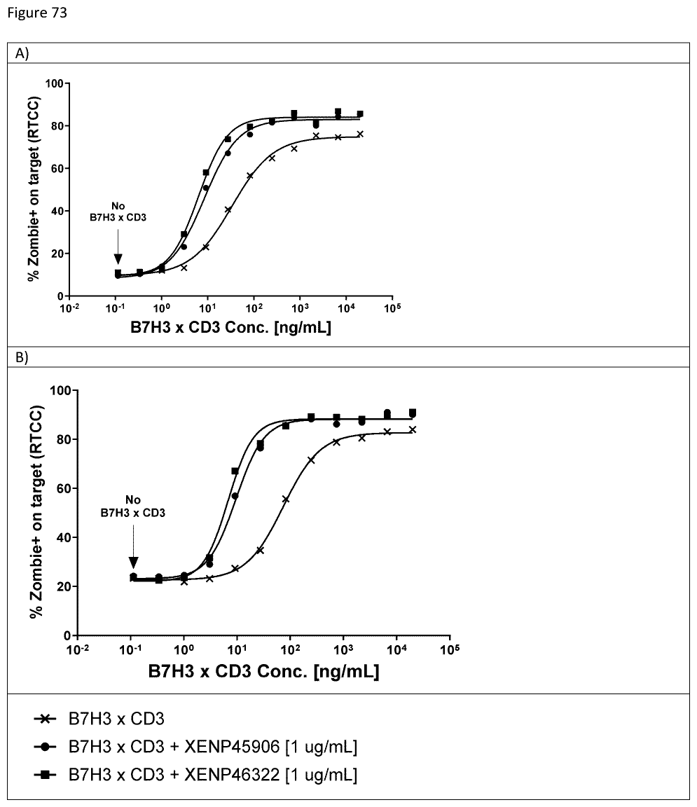

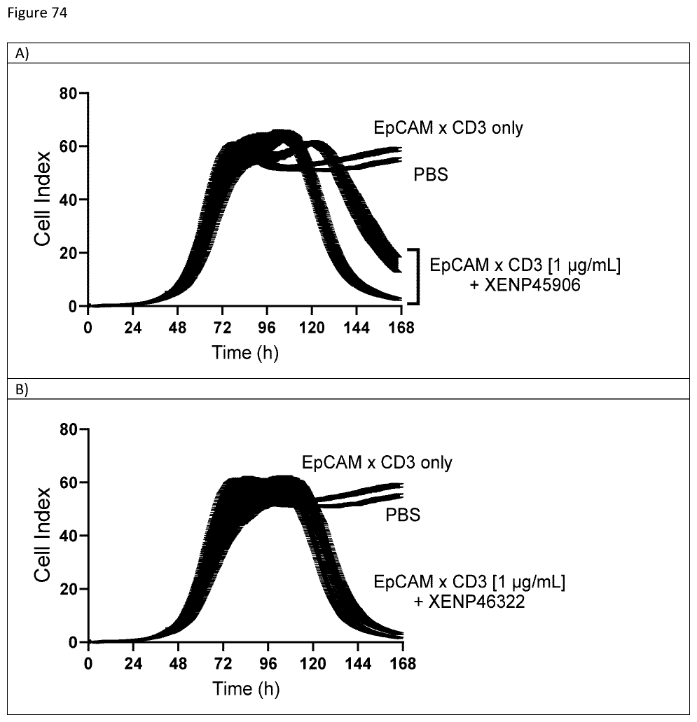

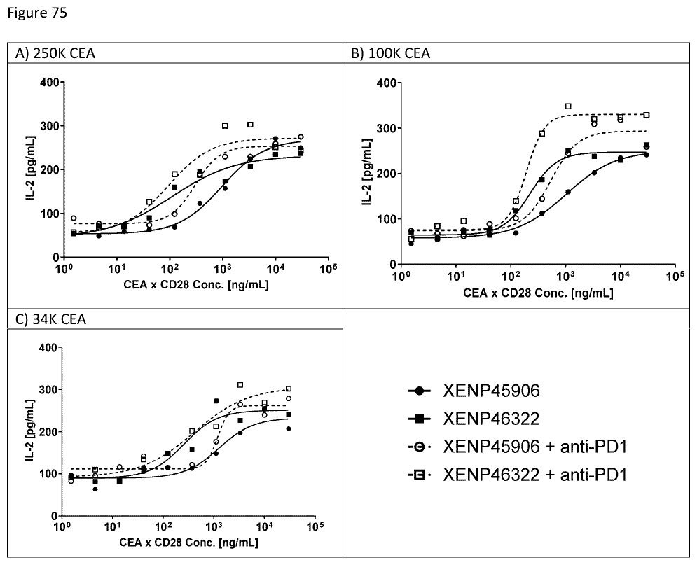

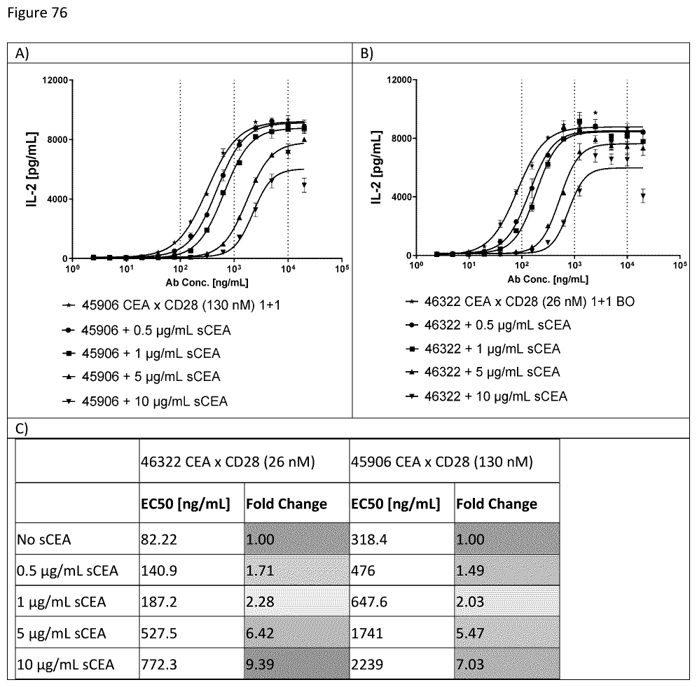

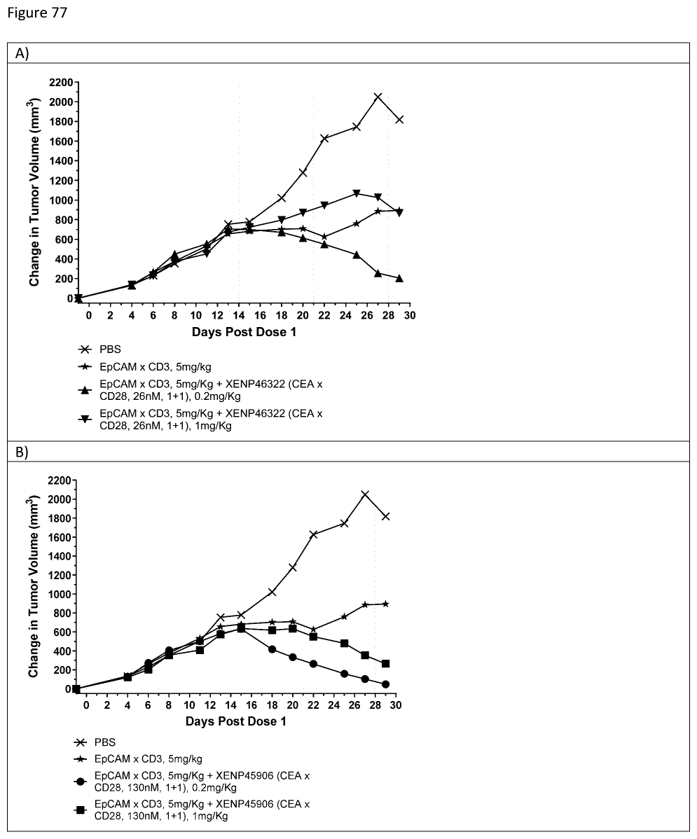

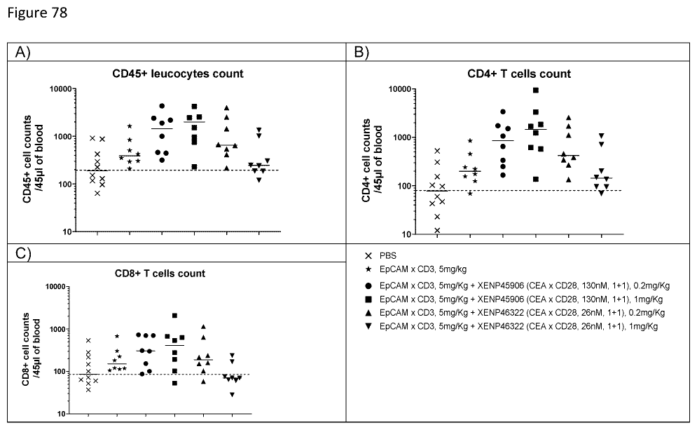

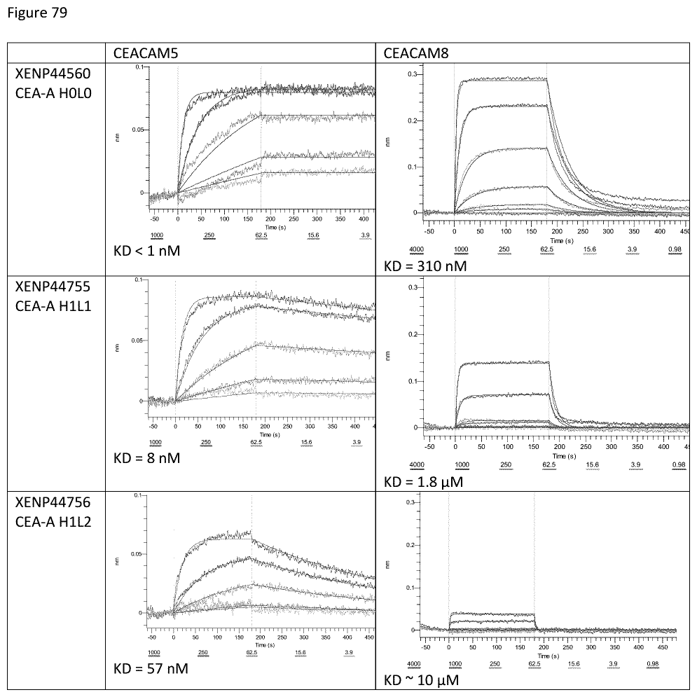

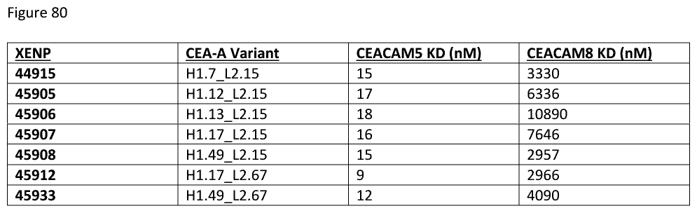

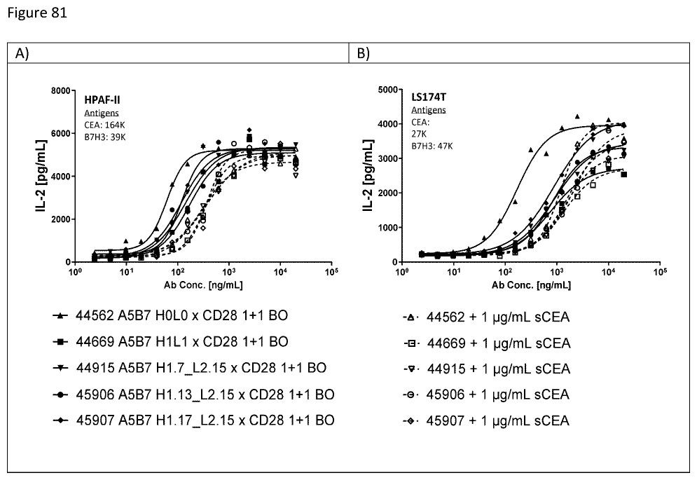

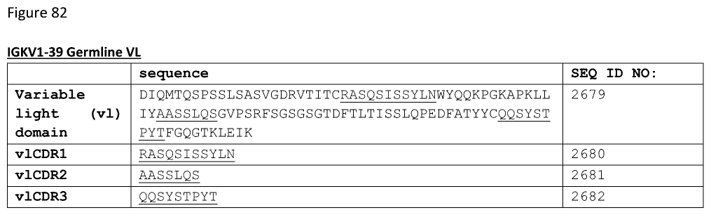

FIG. 10 depicts illustrative sequences ofheterodimeric STEAP1 x CD28 and CEACAM5 x CD28 bsAb backbone for use in the 2+1 mAb-scFv format. The format depicted here is based on heterodimeric Fc backbone 1 as depicted in FIG. 9, except further including G446_on monomer 1 (−) and G446_/K447_on monomer 2 (+). It should be noted that any of the additional backbones depicted in FIG. 9 may be adapted for use in the 2+1 mAb-scFv format with or without including K447_on one or both chains. It should be noted that these sequences may further include the M428L/N434S variants.

FIG. 11 depicts sequences for “CH1” that find use in embodiments of STEAP1 x CD28 bsAbs.

FIG. 12 depicts sequences for “hinge” that find use in embodiments of STEAP1 x CD28 and CEACAM5 x CD28 bsAbs.

FIG. 13 depicts the constant domain of the cognate light chains that find use in the subject STEAP1 x CD28 and CEACAM5 x CD28 bsAb that utilize a Fab binding domain.

FIGS. 14A-14N depicts bispecific formats of the present invention. FIG. 14A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently attached to a light chain constant domain, wherein the light chain variable region is complementary to the VH1. FIG. 14B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 14C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 14D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 14E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CHT-hinge-CH2-CH3, a second monomer comprising VH1-CHT-hinge-CH2-CH3-scFv, and a third monomer comprising VL-CL. The VL pairs with the first and second VH1 to form binding domains with binding specificity for the first antigen. FIG. 14F depicts the “2+1 Fab2-Fc x scFv-Fc” format, with a first Fc comprising two N-terminal Fab arms binding a first antigen and a second Fc domain comprising an N-terminal scFv binding a second antigen. The 2+1 Fab2-Fc x scFv-Fc format comprises a first monomer comprising VH1-CHT-VH2-CH1-hinge-CH2-CH3, a second monomer comprising scFv-domain linker-CH2-CH3, and a third and fourth monomer comprising VL-CL. The VL pairs with the VH1 and VH2 to form binding domains with binding specificity for the first antigen. Additional bispecific formats include G) dual scFv, H) one-arm scFv-mAb, I) scFv-mAb, J) bispecific mAb, K) one-arm central-scFv, L) mAb-Fv, M) central-Fv, and N) trident.

FIG. 15 depicts the variable heavy and variable light chain sequences for 1A7, an exemplary phage-derived CD28 binding domain, as well as the sequences for XENP28428, an anti-CD28 mAb based on 1A7 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 16A-16F depict the sequences for affinity-optimized variable heavy domains from anti-CD28 clone 1A7. It should be noted that the variable heavy domains can be paired with any of the other variable light domains depicted in FIGS. 15, 17 and 18 including SEQ ID NOs: XXX-YYY.

FIGS. 17A-171 depict the sequences for affinity-optimized variable light domain from anti-CD28 clone 1A7. It should be noted that the variable heavy domains can be paired with any of the other variable light domains depicted in FIGS. 15, 16 and 18 including SEQ ID NOs: XXX-YYY.

FIGS. 18A-18C depict the sequence for illustrative affinity-optimized 1A7 VH/VL pairs. It should be noted that these pairs may be formatted as Fabs or as scFvs. Additionally, in the scFv format, these pairs may be formatted in the VH VL orientation or the VLVH orientation.

FIGS. 19A and 19B depict consensus framework regions (FR) and complementarity determining regions (CDRs) (as in Kabat) for anti-CD28 clone 1A7 variable heavy and variable light domain variants.

FIG. 20 depicts illustrative affinity-engineered 1A7 VH/VL pairs and their binding affinities in the context of scFvs (in the context of 1+1 Fab-scFv-Fc bsAb format).

FIGS. 21A-21H depict the variable heavy and variable light chain sequences for additional CD28 binding domains which find use in the STEAP1 x CD28 and CEACAM5 x CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 22 depicts the variable heavy and variable light chain sequences for exemplary STEAP1 binding domains which find use in the STEAP1 x CD28 and CEACAM5 x CD28 bsAbs of the invention. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIGS. 23A and 23B depict A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation. In some embodiments, TAA1 and TAA2 may be different antigens. In some embodiments, TAA1 and TAA2 may be same antigen but different epitopes. In some embodiments, TAA1 and TAA2 may be same antigen and same epitope.

FIGS. 24A-24I depict the sequences for illustrative STEAP1 x CD28 bsAbs in the 1+1 Fab-scFv-Fc format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the STEAP1 x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

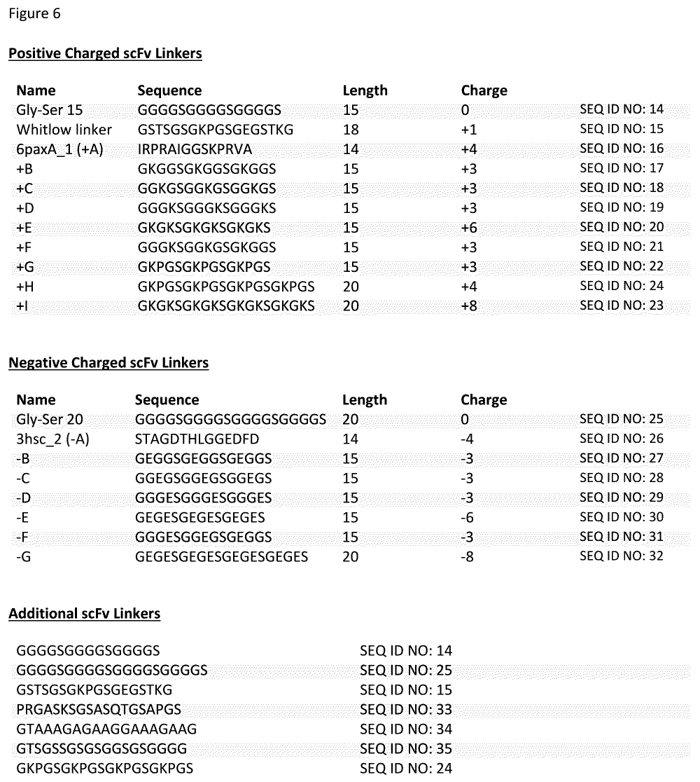

FIGS. 25A-25F depict sequences for exemplary anti-CD3 binding domains suitable for use in CD3 bispecific antibodies which may be combined with the CD28 bispecific antibodies of the invention. The CDRs are underlined, the scFv linker is double underlined (in the sequences, the scFv linker is a positively charged scFv (GKPGS)4 linker (SEQ ID NO: 24), although as will be appreciated by those in the art, this linker can be replaced by other linkers, including uncharged or negatively charged linkers, some of which are depicted in FIG. 6), and the slashes indicate the border(s) of the variable domains. In addition, the naming convention illustrates the orientation of the scFv from N- to C-terminus.

As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 26 depicts the induction of IL-2 secretion by STEAP1 x CD28 bsAb XENP44562 in combination with a constant dose of 1 μg/ml illustrative EpCAM x CD3 bsAb. Purified T cells were incubated with 22Rv1-STEAP1 tumor cells at 1:10 effector:target (E:T) ratio and test articles for 24 hours, and cell supematants were assayed by MSD for IL-2.

FIGS. 27A-27Q depict illustrative STEAP1 x CD28 bsAbs in the 2+1 mAb-scFv-Fc format.

FIG. 28 depicts an exemplary STEAP1 x CD28 in the 2+1 Fab2-Fc x scFv-Fc format.

FIG. 29 depicts the ability of XENP46214, a STEAP1 x CD28 bsAb in the 1 +1 Fab-scFv-Fc format to conditionally enhance T cell activation in the presence of STEAP1 antigen when used in combination with an illustrative B7H3 x CD3 engager.

FIGS. 30A-30CZ depict amino acid sequences for STEAP-A[STEAP1 ]variants in a bivalent mAb format. CDRs are underlined and slashes indicate the border(s) of variable regions.

FIGS. 31A-31U depict engineered variants of the variable heavy domain of the STEAP-A[STEAP-1] Fv.

FIGS. 32A-32J depict engineered variants of the variable light domain of a STEAP-A[STEAP-1] Fv.

FIG. 33 depicts the results of an experiment in which co-cultures of T cells and 22Rv1-STEAPT cancer cells (1:1 E:T ratio) were treated with 0.5 pg/mL of illustrative B7H3 x CD3 bsAb either alone or in combination with 1 μg/mL of a STEAP1 x CD28 bsAb. Cell killing was tracked and analyzed over a period of 6 days via Incucyte. As illustrated, in combination with B7H3 x CD3, STEAP1 x CD28 bsAbs significantly enhanced target cell killing.

FIGS. 34A-34D depict the C4-2B cell binding data from the second round of affinity engineering, sorted by maximum MFI. It illustrates both the successful improvement of cell binding as well as the unpredictable nature of combining various single point mutations.

FIGS. 35A and 35B depict the results of an experiment in which co-cultures of T cells and 22Rv1 cells (1:1 E:T ratio) were treated with 1 μg/mL of a STEAP1 x CD28 bsAb or an illustrative B7H3 x CD28 bsAb in combination with A) 10 μg/mL of illustrative PSMA x CD3 bsAb or B) 10 μg/mL of illustrative STEAP1 x CD3 bsAb. Cell killing was tracked and analyzed over time via Incucyte. As illustrated, STEAP1 x CD28 bsAbs induced significantly better tumor cell killing when compared with illustrative B7H3 x CD28 bsAb.

FIG. 36 depicts avid binding of STEAP1 x CD28 bsAb in the 2+1 mAb-scFv format as well as the more potent induction of IL-2 in the 2+1 mAb-scFv format.

FIG. 36 also depicts the higher antigen binding capacity of STEAP1 x CD28 bsAbs in the 1 +1 Fab-scFv-Fc format.

FIG. 37 depicts the induction of IL2 secretion from purified T cells incubated with 22RV1-STEAP1 tumor cells at a 1:1 effector:target ratio and 1 μg/ml of the indicated illustrative CD3 bispecific and dose escalation of STEAP1 x CD28 bsAb.

FIG. 38 depicts T cell proliferation as indicated by CD4⁺ T cell counts after purified T cell incubated with 22Rv1 tumor cells at 1:1 effector:target ratio, with 1 μg/ml illustrative A) B7H3 x CD3 bsAb and B) PSMA x CD3 bsAb and dose escalation of the indicated STEAP1 x CD28 bsAb.

FIG. 39 depicts the variable heavy, variable light, and CDR sequences for STEAP1 binding domains [STEAP-A]_H1.230_L1.100 and [STEAP-A]_H1.230_L1.115.

FIG. 40 depicts an exemplary STEAP1 x CD28 bsAb in the 2+1 central-scFv format.

FIGS. 41A-41G depict the variable heavy and variable light chain sequences for exemplary CEACAM5 binding domains which find use in the CEACAM5 x CD28 bsAbs of the invention. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIGS. 42A-42YYY depict the sequences for illustrative CEACAM5 x CD28 bsAbs in the 1+1 Fab-scFv-Fc format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the CEACAM5 x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIGS. 43A-43F depicts induction of IL2 secretion from purified T cell incubated with A) MKN45, B) HPAF-I1, C) LS1034, D) LS174T, E) Lovo, or F) HT-29 tumor cells at indicated effector:target (E:T) ratio and 1 μg/ml indicated CEACAM5 x CD28 bsAbs and dose escalation illustrative B7H3 x CD3 bsAb.

FIGS. 44A-44F depicts induction of IL2 secretion from purified T cell incubated with A) LS1034, B) HPAF-II, C) Lovo, D) LS174T, E) SNU-C1, or F) HT-29 tumor cells and 1 μg/ml illustrative B7H3 x CD3 bsAbs and dose escalation indicated CEACAM5 x CD28 bsAbs.

FIGS. 45A-45S depicts humanized CEACAM5 binding domain CEA-A, illustrative affinity-engineered VH and VL variants, as well as consensus framework regions (FR) and complementarity determining regions (CDRs) for the humanized and affinity-engineered variants.

FIG. 46A-46D depicts novel 1A7 VHs affinity-engineered to be paired with IGKV1-39 germline sequence (although they may also be paired with any 1A7 VL), as well as consensus framework regions (FR) and complementarity determining regions (CDRs).

FIG. 47 depicts affinity of novel 1A7 variants paired with IGKV1-39 human germline VL for human CD28.

FIG. 48A-48J depict the sequences for illustrative CEA x CD28 bsAbs in the 2+1 mAb-scFv format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the CEA x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIG. 49A-49M depict the sequences for illustrative CEA x CD28 bsAbs in the 2+1 Fab₂-scFv-Fc format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the CEA x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIG. 50A-50H depict the sequences for illustrative CEA x CD28 bsAbs in the 2+1 Fab₂-Fc x scFv-Fc format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the CEA x CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIGS. 51A-51D depict induction T cell activation (as indicated by CD25 expression on A) CD4 T cells and B) CD8 T cells) and T cell proliferation (as indicated by C) CD4 and D) CD8 T cell counts) after purified T cell incubated with SNU-C1 tumor cells at 1:1 effector:target ratio and 1 μg/ml illustrative B7H3 x CD3 bsAb and dose escalation of XENP44338.

FIGS. 52A-52E depict induction of IL2 secretion from purified T cell incubated with A) HPAF-II (CEAhigh), B) MKN45 (CEAhigh), C) LS1074, D) LS174T (CEAmed-low), and E) HT-29 (CEAlow) tumor cells at 1:1 effector:target ratio and 1 μg/ml illustrative B7H3 x CD3 bsAb and dose escalation of indicated CEA x CD28 bsAbs having CD28 affinity ladder.

FIGS. 53A-53E depict induction of IL2 secretion from purified T cell incubated with A) HPAF-II (CEA^{high}), B) SNU-C1 (CEA^{high}), C) LS1034 (CEA^{med}), D) LS513 (CEA^{med-low}), and E) LS174T (CEA^{med-low}) tumor cells at indicated effector:target ratio and 1 μg/ml illustrative B7H3 x CD3 bsAb and dose escalation of indicated CEA x CD28 bsAbs in 1+1 Fab-scFv-Fc, 2+1 mAb-scFv, or 2+1 Fab₂-scFv-Fc formats.

FIG. 54 depicts EC50 of induction of IL2 secretion from purified T cell incubated with LS174T (CEAmea-^{low}) tumor cells at 1:1 effector:target ratio and 1 μg/ml illustrative B7H3 x CD3 bsAb and dose escalation of indicated CEA x CD28 bsAbs in 1+1 Fab-scFv-Fc or 2+1 Fab2-Fc x scFv-Fc formats with increasing concentrations of soluble CEA. The data show that while the 2+1 format is less efficacious and less potent, it better tolerates soluble CEA as indicated by more sCEA needed to shift EC50 from baseline.

FIGS. 55A-55C depicts mechanism-based modeling of trimer formation (i.e. trimer of bispecific antibody, T cell, and tumor cell) for bsAbs having a) 0.2 nM CEA binding domain, b) 2 nM CEA binding domain, and c) 15 nM CEA binding domain and increasing concentrations of soluble CEA.

FIG. 56 depicts induction of IL2 secretion from purified T cell incubated with HPAF-II tumor cells at 1:1 effector:target ratio and 1 μg/ml illustrative B7H3 x CD3 bsAb and dose escalation of indicated CEA x CD28 bsAbs having CEA-A H0L0 (˜0.7-2 nM), CEA-AH1L1 (˜5-8 nM), or CEA-AH1L2 (57-69 nM) with or without 1 μg/ml soluble CEA.

FIG. 57 depicts induction of IL2 secretion from purified T cell incubated with HPAF-II (CEA^{high}) tumor cells at 1:1 effector:target ratio and 1 μg/ml illustrative B7H3 x CD3 bsAb and dose escalation of indicated CEA x CD28 bsAbs having affinity-engineered CEA-A H1L1 variants (with or without 1 μg/ml sCEA).

FIG. 58 depicts induction of IL2 secretion from purified T cell incubated with HPAF-II (CEA^{high}) tumor cells at 1:1 effector:target ratio and 1 μg/ml illustrative B7H3 x CD3 bsAb and dose escalation of indicated CEA x CD28 bsAbs having affinity-engineered CEA-A H1L2 variants (with or without 1 μg/ml sCEA).

FIG. 59 depicts induction of IL2 secretion from purified T cell incubated with HPAF-II (CEA^{high}) tumor cells at 1:1 effector:target ratio and 1 μg/ml illustrative B7H3 x CD3 bsAb and dose escalation of indicated CEA x CD28 bsAbs having affinity-engineered CEA-A H1L1 and H1L2 variants.

FIG. 60 depicts induction of IL2 secretion from purified T cell incubated with HPAF-II (CEA^{high}) tumor cells at 1:1 effector:target ratio and 1 μg/ml illustrative B7H3 x CD3 bsAb and dose escalation of indicated CEA x CD28 bsAbs having affinity-engineered CEA-A H1L1 and H1L2 variants (with or without 1 μg/ml sCEA).

FIGS. 61A and 61B depict induction of IL2 secretion after A) 6 hours or B) 24 hours from purified T cell incubated with MKN-45 (CEA^{high}) or Lovo (CEA° ⁰) cells at 1:1 effector:target ratio with dose escalation of indicated CEA x CD28 bsAbs.

FIG. 62 depicts induction of IL2 secretion from purified T cell incubated with LS1034 tumor cells at 10:1 effector:target ratio with or without 20 nM PD-1 mAb and dose escalation of indicated CEA x CD28 bsAbs.

FIG. 63 depicts increase in thermostability of disulfide-stabilized 1A7 scFv.

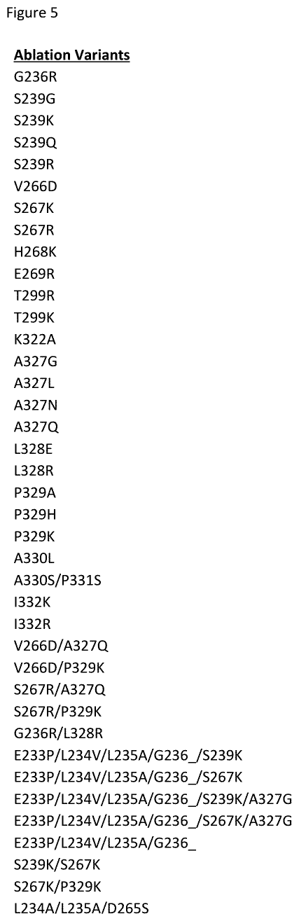

FIG. 64 depicts CD28 affinity for additional 1A7 scFv variants, with or without disulfide stabilization (as denoted by [S-S]) obtained from different experiments in the context of different CD28 multispecific antibodies. While all scFvs depicted below used the scFv linker (GKPGS)4 (SEQ ID NO: 24), any scFv linker (including those in FIG. 5), can be used. Additionally, the scFv can be in either orientation, VH-scFv linker-VL or VL-scFv linker-VH.

FIGS. 65A-65E depict illustrative 1A7-based scFvs with engineered cysteines for disulfide stabilization. While all scFvs depicted below used the scFv linker (GKPGS)4 (SEQ ID NO: 24), any scFv linker (including those in FIG. 5), can be used.

Additionally, the scFv can be in either orientation, VH-scFv linker-VL or VL-scFv linker-VH, although only the former is shown.

FIG. 66 depicts induction of IL2 secretion from purified T cell incubated with HPAF-II tumor cells at 1:1 effector:target ratio and 1 μg/ml illustrative B7H3 x CD3 bsAb and dose escalation of indicated CEA x CD28 bsAbs having CD28 scFv with or without disulfide-stabilization.

FIG. 67 depicts A431-02M-null cells stably expressing CEACAM5 were engineered to express a CMV-derived (HLA-A2/NLVa) or a melanoma-derived (HLA-A2/G209; negative control) pMHC antigenic complex. Cells were cocultured with PBMCs from an HLA-A2⁺, CMV-seropositive donor, and treated with a dose titration of CEACAM5 x CD28 XENP45906.

FIG. 68 depicts induction of IL2 from purified T cell incubated with MKN45 (CEAhigh), HPAF-II (CEAhigh), LS1034 (CEAmed), LS174T (CEAmed), Lovo (CEAme^{d}), and HT29 (CEA° ^{W}) cells at 1:1 effector:target ratio and 1 μg/ml illustrative B7H3 x CD3 bsAb and dose escalation of CEA x CD28 XENP44915.

FIGS. 69A and 69B depict induction of A) CD8 T cell activation (as indicated by CD25 expression) and B) tumor cell killing after incubating purified T cells with HPAF-II (CEA^{high}) cells and 0.1 or 1 μg/ml illustrative B7H3 x CD3 bsAb and dose escalation of CEA x CD28 XENP44915. The data show that CEA x CD28 synergizes with high and low concentrations of T-cell engager.

FIGS. 70A-70F depict induction of IL2 secretion from purified T cell incubated with A) MKN45, B) HPAF-II, C) LS1034, D) LS174T, E) Lovo, and F) HT-29 cells at 1:1 effector:target ratio and 1 μg/ml illustrative B7H3 x CD3 bsAb and dose escalation of indicated CEA x CD28 bsAbs having CD28 scFv with or without disulfide-stabilization and with CEA binding domains having varying affinities.

FIGS. 71A-71D depict induction of IL2 secretion from purified T cell incubated with A) MKN-45, B) LS174T, C) Lovo, and D) HT-29 tumor cells at 1:1 effector:target ratio and 1 μg/ml illustrative B7H3 x CD3 bsAb and dose escalation of indicated CEA x CD28 bsAbs in 1+1 Fab-scFv-Fc or 2+1 Fab₂-Fc x scFv-Fc format.

FIGS. 72A-72B depict induction of IL2 secretion from purified T cell incubated with indicated tumor cells at 1:1 effector:target ratio and 1 μg/ml illustrative B7H3 x CD3 bsAb and dose escalation of A) XENP45906 or B) XENP46322.

FIG. 73 depicts induction of A) LS-174T or B) Lovo cancer cell kill by purified T cell incubated at 1:1 effector:target ratio and 1 μg/ml CEACAM5 x CD28 bsAbs XENP45906 or XENP46322 and dose escalation illustrative B7H3 x CD3 bsAb.

FIG. 74 depicts induction of HPAF-II cancer cell kill by purified T cell incubated at 1:1 effector:target ratio and CEACAM5 x CD28 bsAbs A) XENP45906 or B) XENP46322 and dose illustrative EpCAM x CD3 bsAb.

FIG. 75 depicts induction of IL2 secretion by purified CMV-reactive T cell incubated at 10:1 effector:target ratio with A431 HLA-A2/NLV CEA knock-in cells with A) 250K, B) 100K, and C) 34K CEACAM5 antigen density and dose escalation CEACAM5 x CD28 bsAbs XENP45906 or XENP46322 alone or with anti-PD1.

FIG. 76 depicts induction of IL2 secretion by purified T cell incubated at 1:1 effector:target ratio with HPAF-II and dose escalation CEACAM5 x CD28 bsAbs A) XENP45906 or B) XENP46322 with 1 pg B7H3 x CD3 with or without varying concentrations of soluble CEA. C) Fold Change.

FIG. 77 depicts the change in tumor volume (as determined by caliper measurements) over time in HPAF-II and huPBMC-engrafted NSG-DKO mice dosed with A) XENP46322 or B) XENP45906 with illustrative EpCAM x CD3 bsAb.

FIG. 78 depicts A) human CD45⁺, B) human CD4⁺ and C) human CD8⁺ cell counts on Day 21 in HPAF-II and huPBMC-engrafted NSG-DKO mice dosed with XENP46322 or XENP45906 with illustrative EpCAM x CD3 bsAb.

FIG. 79 depicts Octet sensorgrams 25° C. showing binding of CEACAM5 x CD28 bsAbs having CEA-A_H0L0, CEA-A H1L1, or CEA-A_H1L2 to CEACAM5 and CEACAM8.

FIG. 80 depicts binding of CEACAM5 x CD28 bsAbs having further engineered variants of CEA-A_H1.7_L2.15 to CEACAM5 and CEACAM8 as determined by Octet at 25° C.

FIG. 81 depicts induction of IL2 secretion by purified T cells in the presence of A) HPAF-II or B) LS174T cancer cells and dose titration of CEACAM5 x CD28 engineered for reduced for reduced CEACAM8 cross reactivity and 1 μg/ml B7H3 x CD3 bsAb with or without 1 μg/ml soluble CEA.

FIG. 82 depicts sequence for IGKV1-39 human germline (CDRs underlined). These may be paired with any of the 1A7 depicted herein, although preferable pairs are those depicted in FIG. 47.

### DETAILED DESCRIPTION

### I. Overview

Six-Transmembrane Epithelial Antigen of the Prostate 1 (STEAP1 ) is a cell surface antigen that functions in cellular iron homeostasis. Carcinoembryonic antigen-related cell adhesion molecule 5 (CEACAM5) is a cell surface glycoprotein, and member of the carcinoembryonic (CEA) antigen family. As STEAP1 and CEACAM5 are overexpressed on a variety of tumors, these tumor antigens are candidates for targeted therapeutic development.

The activation of T cells in the treatment of cancer is being widely investigated. T cells require multiple signals for complete activation and differentiation. As shown in FIG. 23A, Signal 1, promoted by recognition of a peptide-MHC (pMHC) complex by the T cell receptor (TCR), is absolutely required for T cell activation. Signal 2, which synergizes with, and amplifies signal 1, is typically provided by the interaction of the CD28 ligands CD80 and CD86 with CD28 itself. Although CD28 engagement alone is typically inert, when combined with signal 1 activation, it promotes additional activation, survival, and proliferative signals, including IL-2 secretion. As CD80 and CD86 are only naturally expressed by professional antigen-presenting cells (APC), the extent of CD28 costimulation in the tumor setting can be highly variable. Accordingly, the present invention is directed to a novel class of tumor-targeted anti-CD28 x anti-STEAP1 bispecific antibodies, the CD80/CD86 engagement of CD28 that mimic the CD80/CD86 engagement of CD28, thereby providing an artificial source of signal 2 (FIG. 23B). Notably, signal 1 can either be provided by the natural TCR:pMHC recognition of tumor cells, or it can be provided by combination of the CD28 bispecific with a CD3 bispecific (e.g., anti-CD3 x anti-STEAP1 ), which can mimic signal 1.

Accordingly, provided herein are novel anti-CD28 x anti-STEAP1 (also referred to as “uCD28 x uSTEAP1 ” and sometimes “CD28 x STEAP1 ”) and anti-CD28 x anti-CEACAM5 (also referred to as “uCD28 x uCEACAM5” and sometimes “CD28 x CEACAM5”) bispecific antibodies and methods of using such antibodies for the treatment of cancers. In many cases, these bispecific antibodies are heterodimeric. Subject uCD28 x uSTEAP1 antibodies are capable of agonistically binding to CD28 costimulatory molecules on T cells and targeting to STEAP1 on STEAP1 -expressing tumor cells. Similarly, subject uCD28 x uCEACAM5 antibodies are capable of agonistically binding to CD28 costimulatory molecules on T cells and targeting to CEACAM5 on CEACAM5-expressing tumor cells.

Thus, such antibodies selectively enhance anti-tumor activity at tumor sites that express the target antigen of interest while minimizing peripheral toxicity.

The subject antibodies provided herein are particularly useful for enhancing anti-tumor activity either alone, as a monotherapy, or when used in combination with other anti-cancer therapies as more fully described herein.

Accordingly, in one aspect, provided herein are heterodimeric antibodies that bind to two different antigens, e.g., the antibodies are “bispecific,” in that they bind two different target antigens, generally CD28 and STEAP1 or CD28 and CEACAM5 as described below. These heterodimeric antibodies can bind each of the target antigens either monovalently (e.g., there is a single antigen binding domain such as a variable heavy and variable light domain pair) or bivalently (there are two antigen binding domains that each independently bind the antigen). In some embodiments, the heterodimeric antibody provided herein includes: a) one CD28 binding domain; and b) one STEAP1 or CEACAM5 binding domain (e.g., heterodimeric antibodies in the “1+1 Fab-scFv-Fc” format described herein, which are thus bispecific and bivalent). In other embodiments, the heterodimeric antibody provided herein includes: a) one CD28 binding domain; and b) two STEAP1 or CEACAM5 binding domains (e.g., heterodimeric antibodies in the “2+1 Fab2-scFv-Fc,” “2+1 mAb-scFv” and “2+1 Fab2-Fc x scFv-Fc” formats described herein, which are thus bispecific but trivalent, as they contain three antigen binding domains (ABDs)). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with purification variants (e.g., “pI variants”) that allow simple purification of the heterodimers away from the homodimers, as is similarly outlined below.

The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The naming nomenclature of particular antigen binding domains (e.g., STEAP1 and CD28 binding domains) use a “Hx.xx Ly.yy” type of format, with the numbers being unique identifiers to particular variable chain sequences. Thus, for example, the CD28 binding domain “1A7[CD28]H1 L1” (FIG. 15) includes a variable heavy domain, H1, and a variable light domain L1. In the case that these sequences are used as scFvs, the designation “H1iL1”, indicates that the binding domain includes a variable heavy domain “H1” combined with a variable light domain “L1,” and is in VH-linker-VL orientation, from N- to C-terminus. This molecule with the identical sequences of the heavy and light variable domains but in the reverse order (VL-linker-VH orientation, from N- to C-terminus) would be designated “L1iH1”. Similarly, different constructs may “mix and match” the heavy and light chains as will be evident from the sequence listing and the Figures.

### III. Definitions

In order that the application may be more completely understood, several definitions are set forth below. Such definitions are meant to encompass grammatical equivalents.

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with aYMNM motif (SEQ ID NO: 2683) critical for the recruitment of SH2-domain containing proteins, particularly PI3K. CD28 also includes two proline-rich motifs that are able to bind SH3-containing proteins. Exemplary CD28 sequences are depicted in FIG. 1. Unless otherwise noted, references to CD28 are to the human CD28 sequence.

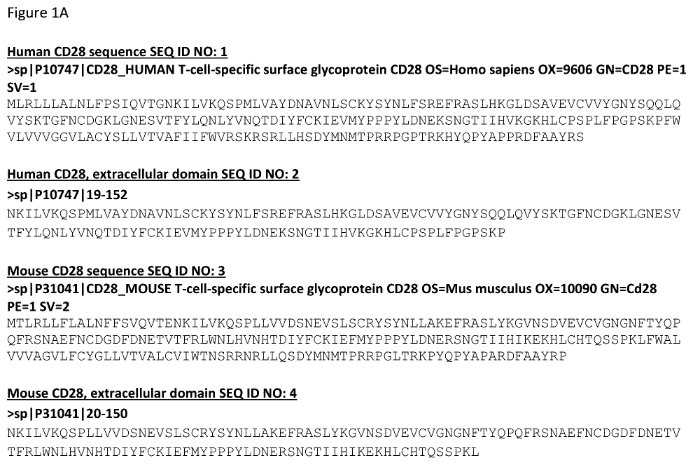

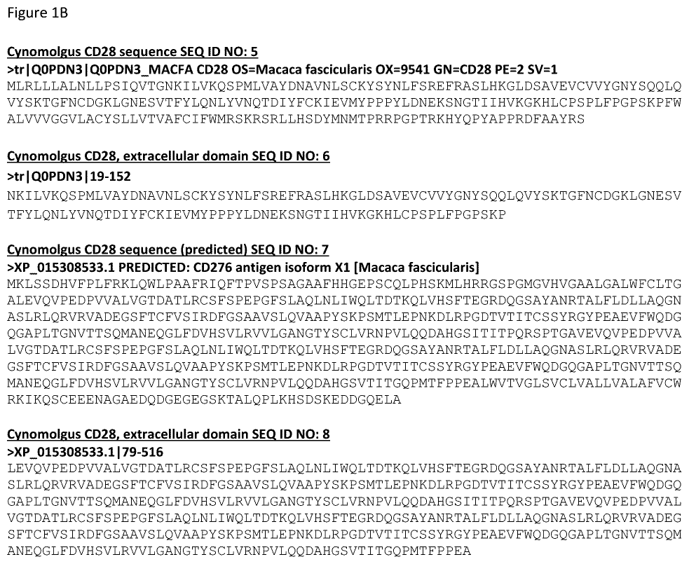

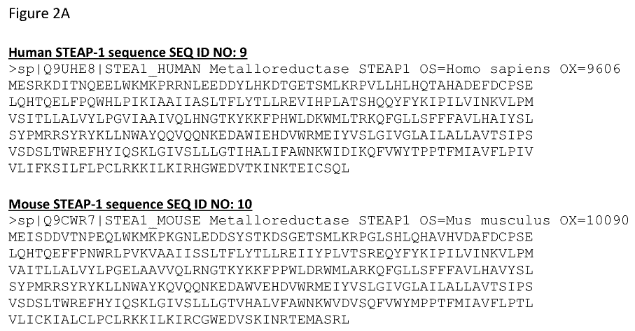

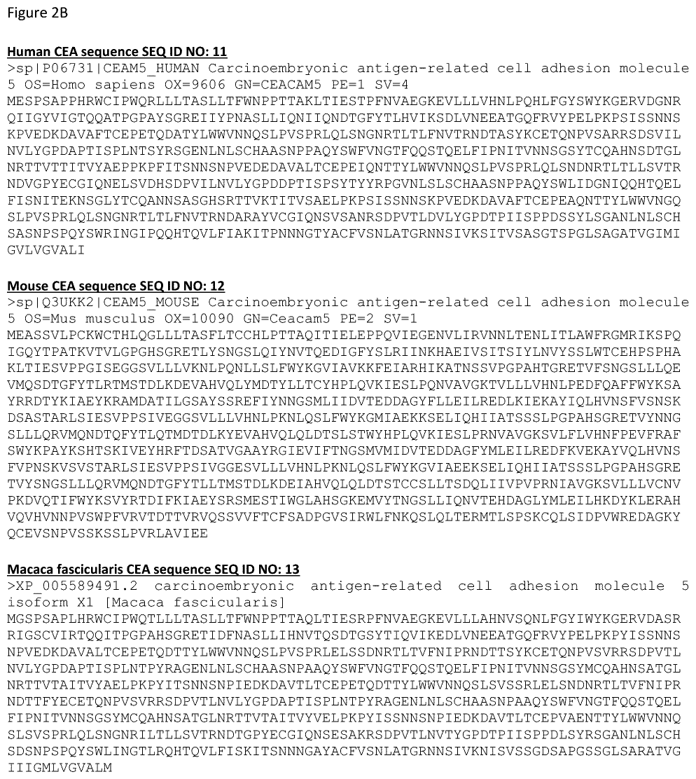

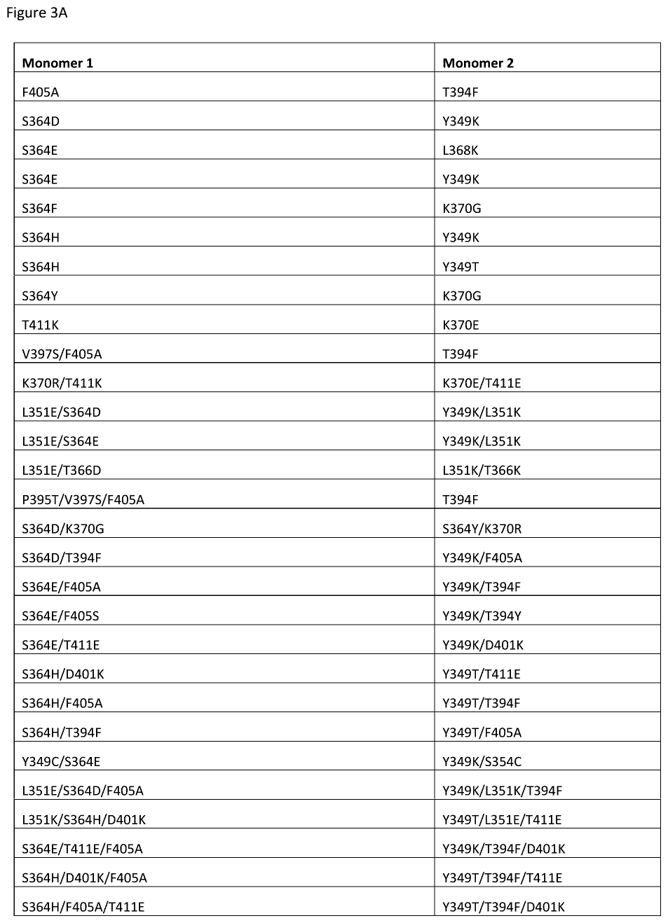

By “STEAP1 ,” “PRSS24,” “six transmembrane epithelial antigen of the prostate 1,” or “STEAP family member 1” as used herein is meant a metalloproteinase that functions in in iron and cooper homeostasis, and includes six transmembrane domains, three extracellular loops and two intracellular loops. STEAP1 is expressed in particular cancers, including prostate cancer, wherein STEAP1 is overexpressed in malignant prostate tissue relative to normal prostate tissue. Exemplary STEAP1 sequences are depicted in FIG. 2.

By “CEACAM5,” “carcinoembryonic antigen-related cell adhesion molecule 5,” “CEA cell adhesion molecule 5,” “CD66e” or “Cluster of Differentiation 66e” or “CEA” herein is member of the carcinoembryonic antigen (CEA) family of cell surface glycoproteins involved in cell adhesion. CEACAM5 sequences are depicted, for example, in FIG. 2. CECAM is overexpressed in a variety of carcinomas, including those of the gastrointestinal tract, the respiratory and genitourinary systems, and breast cancer.

By “ablation” as used herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 5, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

Other useful antibody formats include, but are not limited to, the “1+1 Fab-scFv-Fc,” and “2+1 Fab2-scFv-Fc,” “2+1 mAb-scFv,” and “2+1 Fab2-Fc x scFv-Fc” formats provided herein (see, e.g., FIG. 14). Additional useful antibody formats include, but are not limited to, “1+1 common light chain,” and “2+1 common light chain,” “mAb-Fv,” “mAb-scFv,” “central-Fv”, “one-armed scFv-mAb,” “scFv-mAb,” “dual scFv,” and “trident” format antibodies (FIG. 14). See also, US20180127501A1, which is incorporated by reference herein, particularly in pertinent part relating to antibody formats (see, e.g., FIG. 2 of US20180127501A1).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CHT-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains.

In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cy2 and Cy3), and optionally all or a portion of the hinge region between CH1 (Cy1) and CH2 (Cy2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CHT-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or CK). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., STEAP1 , CEACAM5, or CD28) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the Vx, VW, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDRT-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CHT on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example,—233E or 233E designates an insertion of glutamic acid after position 233 and before position 234. Additionally,—233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 #designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, Clq, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match,” heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type,” “wild-type,” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T.F. & Waterman, M.S. (1981) “Comparison Of Biosequences,” Adv. Appl. Math. 2:482 [local homology algorithm]; Needleman, S.B. & Wunsch, CD. (1970) “A General Method Applicable To The Search For Similarities In The Amino Acid Sequence Of Two Proteins,” J. Mol. Biol.48:443 [homology alignment algorithm], Pearson, W.R. & Lipman, D.J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

IV. Anti-CD28 xAnti-STEAP1 Antibodies

In one aspect, provided herein are novel anti-CD28 x anti-STEAP1 antibodies In some embodiments, the anti-CD28 x anti-STEAP1 antibodies described herein are capable of agonistically binding to CD28 costimulatory molecules on T cells and STEAP1 on tumor cells. Such antibodies selectively enhance anti-tumor activity at STEAP1 -associated tumor sites while minimizing peripheral toxicity. The subject antibodies provided herein are particularly useful in combination with other anti-cancer therapies, including, for example, bispecific antibodies for the treatment of STEAP1 -associated cancers.

The anti-CD28 x anti-STEAP1 antibodies are multivalent and include at least two antigen binding domains (ABDs), wherein at least one antigen binding domain is a CD28 binding domain and at least one antigen binding domain is a STEAP1 binding domain. Any suitable CD28 binding domain and STEAP1 binding domain can be included in the subject anti-CD28 x anti-STEAP1 antibodies, including, for example, the CD28 binding domains and STEAP1 binding domains provided herein.

The antigen binding domains provided herein generally include a variable heavy domain (VH) having a VH-CDR1, VH-CDR-2, and VH-CDR-3; and a variable light domain (VL), and a variable light domain (VL) having a VL-CDR1, VL-CDR-2, and VL-CDR-3.

In addition, as discussed above, the numbering used in the sequence listing and figures for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85, 90, 95 or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85, 95 to 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain).

In addition, in embodiments wherein the subject antibody includes an scFv, the scFv can be in an orientation from N- to C-terminus ofVH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIG. 6.

In some embodiments, the anti-CD28 x anti-STEAP1 antibody is a bispecific antibody. In some embodiments, the anti-CD28 x anti-STEAP1 antibody is a bivalent antibody. In some embodiments, the anti-CD28 x anti-STEAP1 antibody is a trivalent antibody. In some embodiments, the anti-CD28 x anti-STEAP1 antibody is a bispecific, bivalent antibody. In some embodiments, the anti-CD28 x anti-STEAP1 antibodies include one CD28 binding domain and one STEAP1 binding domain. In exemplary embodiments, the anti-CD28 x anti-STEAP1 antibody is a bispecific, trivalent antibody. In some embodiments, the anti-CD28 x anti-STEAP1 antibodies include one CD28 binding domain and two STEAP1 binding domains.

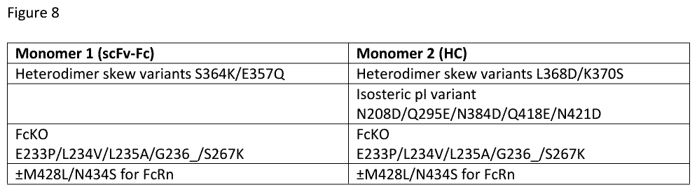

The anti-CD28 x anti-STEAP1 antibodies provided herein can be in any useful format, including, including, for example, canonical immunoglobulin, as well as the “1+1 Fab-scFv-Fc,” “2+1 “mAb-scFv,” “2+1 Fab2-scFv-Fc,” and “2+1 Fab2-Fc x scFv-Fc” formats described herein (FIG. 14). Additional useful formats include, but are not limited to: “mAb-Fv,” “central-Fv,” “one-armed scFv-mAb,” “scFv-mAb,” “dual scFv,” and “trident” formats provided herein (see, e.g., FIG. 14). See also, US20180127501A1, which is incorporated by reference herein, particularly in pertinent part relating to antibody formats (see, e.g., FIG. 2). In some embodiments, the anti-CD28 x anti-STEAP1 antibodies are heterodimeric bispecific antibodies that include variant Fc domains having any of the heterodimerization skew variants, pI variants and/or ablation variants described herein. See, e.g. FIG. 8.

Note that unless specified herein, the order of the antigen list in the name does not confer structure; that is an anti-STEAP1 x anti-CD28 1+1 Fab-scFv-Fc antibody can have the scFv bind to STEAP1 or CD28, although in some cases, the order specifies structure as indicated.

The anti-CD28 x anti-STEAP1 antibodies provided herein further include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CHT-hinge-Fc domain or CHT-hinge-CH2-CH3), the variable heavy domain, the variable light domain, the light constant domain, Fab domains and scFv domains.

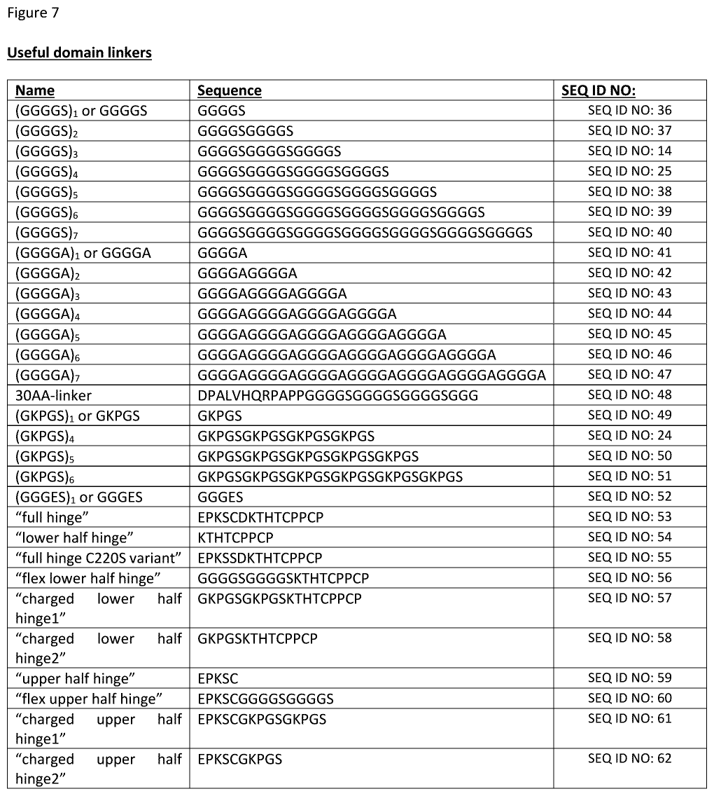

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr.

The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 2684), (GGGGS)n (SEQ ID NO: 36), and (GGGS)n (SEQ ID NO: 2685), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example, the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker,” used to link any two domains as outlined herein together. For example, in the 2+1 Fab2-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 2684), (GGGGS)n (SEQ ID NO: 36), and (GGGS)n (SEQ ID NO: 2685), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness,” as outlined below, charged domain linkers, as used in some embodiments of scFv linkers can be used. Exemplary useful domain linkers are depicted in FIG. 7.

In some embodiments, the linker is a scFv linker that is used to covalently attach the VH and VL domains as discussed herein. In many cases, the scFv linker is a charged scFv linker, a number of which are shown in FIG. 6. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers.

Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the invention as well, and thus those included in FIGS. 6 and 7 can be used in any embodiment herein where a linker is utilized.

Exemplary subject anti-CD28 x anti-STEAP1 antibodies are depicted, for example, in FIG. 24. During the cell culture production of the anti-CD28 x anti-STEAP1 antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-CD28 x anti-STEAP1 antibody is a variant of one of the anti-CD28 x anti-STEAP1 antibodies includes a deletion of a C-terminal lysine (-K) terminal or C-terminal lysine and glycine (-GK) residues in one or both Fc domains of an anti-CD28 x anti-STEAP1 antibody described herein. In some embodiments, the deletion is G446del and/or K447del (EU numbering).

Aspects of the anti-CD28 x anti-STEAP1 antibodies are further described in detail below.

A. CD28 Binding Domains

The anti-CD28 x anti-STEAP1 antibodies provided herein include at least one CD28 binding domain. Any suitable CD28 binding domain can be included in the anti-CD28 x anti-STEAP1 antibodies provided herein. In exemplary embodiments, the CD28 binding domain is an agonistic CD28 ABDs that advantageously provide T cell costimulatory activity.

As will be appreciated by those in the art, suitable CD28 binding domains can comprise a set of 6 CDRs as depicted in the figures, either as they are underlined or, in the case where a different numbering scheme is used, as described herein and as shown in Table 2, as the CDRs that are identified using other alignments within the variable heavy (VH) domain and variable light domain (VL) sequences of those CD28 binding domains depicted in FIGS. 15, 18, and 21. Additional VH and VL sequences of exemplary CD28 binding domains that can be used in the subject antibodies are depicted in FIGS. 16 and 17. Suitable CD28 ABDs can also include the entire VH and VL sequences as depicted in these sequences and figures, used as scFvs or as Fabs.

In one embodiment, the CD28 antigen binding domain includes the 6 CDRs (i.e., vhCDR1-3 and vlCDR1-3) of any of the CD28 binding domains described herein, including, but not limited to those depicted in FIGS. 15, 18, 21 and 65. In some embodiments, the CD28 ABD that binds human CD28 is one of the following CD28 ABDs: 1A7[CD28]_H1L1, 1A7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, 1A7[CD28]_H1.1_L1 [S-S], 1A7[CD28]_H1_L1.71 [S-S], 1A7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the CD28 ABD includes a VH/VL pair selected from the VHs and VLs depicted in FIGS. 15, 16, 17, and 82.

In addition to the parental CDR sets disclosed in the figures and sequence listing that form an ABD to CD28, provided herein are variant CD28 ABDS having CDRs that include at least one modification of the CD28 ABD CDRs disclosed herein (e.g., (FIGS. 15, 18, 21 and 65, and the sequence listing). In one embodiment, the CD28 ABD of the subject anti-CD28 x anti-STEAP1 antibody includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD of the subject anti-CD28 x anti-STEAP1 antibody includes a set of 6 CDRs with 1, 2, 3, 4, 5, 6, 7, 8, 9, 10 amino acid modifications as compared to the 6 CDRs of one of the following CD28 ABDs: 1A7[CD28]_H1L1, 1A7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, 1A7[CD28]_H1.1_L1 [S-S], 1A7[CD28]_H1_L1.71 [S-S], 1A7[CD28]_H1.1_L1.71 [S-S], 1A7[CD28]_H1.14_L1 [S-S], 1A7[CD28]_H1.14_L1.71 [S-S], 1A7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65). In certain embodiments, the CD28 ABD of the subject anti-CD28 x anti-STEAP1 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 ABD of the subject anti-CD28 x anti-STEAP1 antibody includes 6 CDRs that are at least 90, 95, 97, 98 or 99% identical to the 6 CDRs of a CD28 ABD as described herein, including the figures and sequence listing. In exemplary embodiments, the CD28 ABD of the subject anti-CD28 x anti-STEAP1 antibody includes 6 CDRs that are at least 90, 95, 97, 98 or 99% identical to the 6 CDRs of one of the following CD28 ABDs: IA7[CD28]_H1L1, IA7[CD28]_H1.1_L1, IA7[CD28]_H1iL1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In another exemplary embodiment, the CD28 ABD of the subject anti-CD28 x anti-STEAP1 antibody includes the variable heavy (VH) domain and variable light (VL) domain of any one of the CD28 ABDs described herein, including the figures and sequence listing. In exemplary embodiments, the CD28 ABD is one of the following CD28 ABDs: IA7[CD28]_H1L1, IA7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the CD28 ABD includes a VH and VL pair selected from the VH and VLs depicted in FIGS. 15, 16, 17, and 82.

In some embodiments, the anti-CD28 x anti-STEAP1 antibody includes a CD28 ABD that includes a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD28 ABDs: IA7[CD28]_H1L1, IA7[CD28]_H1.1_L1, IA7[CD28]_H1_L1.71, IA7[CD28]_H1.1_L1.71, IA7[CD28]_H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], 1A7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65). In some embodiments, the changes are in a VH domain depicted in FIGS. 15, 18, 21 and 65. In some embodiments, the changes are in a VL domain are depicted in FIGS. 15, 18, 21 and 65. In some embodiments, the changes are in a VH and VL domain are depicted in FIGS. 15, 18, 21 and 65. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of one of the following CD28 ABDs: IA7[CD28]_H1L1, IA7[CD28]H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the CD28 ABD includes a VH that is at least 90, 95, 97, 98 or 99% identical to VH domain depicted in FIGS. 15-18, 21 and 65. In some embodiments, the CD28 ABD includes a VL that is at least 90, 95, 97, 98 or 99% identical to VL domain depicted in FIGS. 15-18 and 21. In some embodiments, the CD28 ABD includes a VH and a VL that is at least 90, 95, 97, 98 or 99% identical to a VH domain and a VL domain depicted in FIGS. 15-18, 21 and 65. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 binding domain of the subject anti-CD28 x anti-STEAP1 antibody includes a VH that includes any one of the VHCDRI-3 or HFR1-4 sequences depicted in FIG. 19A. In some embodiments, the CD28 binding domain includes a VL that includes any one of the VLCDRI-3 or LFR1-4 sequences depicted in FIG. 19B.

In some embodiments, the anti-CD28 x anti-STEAP1 antibody includes a CD28 binding domain that includes a VH and VL selected from the following:(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domains depicted in FIGS. 15, 18, 21 and 65 or a variant thereof; or(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIGS. 15, 17 or 82 or a variant thereof.

In some embodiments, the anti-CD28 x anti-STEAP1 antibody includes a CD28 binding domain that includes a VH and VL selected from the following:(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or(i) a VH having an amino acid sequence of a VH depicted in FIG. 15 or 16 or a variant thereof; and (ii) a VL having an amino acid sequence of the VL of a VL depicted in FIGS. 15, 17 or 82 or a variant thereof.

### B. STEAP1 Binding Domains

In one aspect, provided herein are STEAP1 antigen binding domain (ABD) compositions. In embodiments, the STEAP1 ABDs include a set of 6 CDRs as depicted in the sequence listing and FIG. 22 or FIGS. 31 and 32, either as the CDRs are underlined or, in the case where a different numbering scheme is used as described herein and as shown in Table 2, as the CDRs that are identified using other alignments within the variable heavy (VH) domain of those depicted in FIGS. 22, 31, or 39 and variable light domain (VL) sequences of those depicted in FIGS. 22, 32, or 39 and the sequence listing (see Table 2).

Suitable STEAP1 ABDs can also include the entire VH and VL sequences as depicted in these sequences and figures, used as scFvs or as Fab domains. In embodiments, the STEAP1 ABD includes a variable heavy (VH) domain or variant thereof and/or variable light (VL) domain or variant thereof of any of the STEAP1 binding domains disclosed herein (e.g., FIGS. 22 and 39). Such STEAP1 binding domains are useful, for example, for inclusion in the anti-CD28 x anti-STEAP1 antibodies provided herein.

In exemplary embodiments, the STEAP1 ABD includes the variable heavy (VH) domain and variable light (VL) domain of any one of the STEAP1 ABDs described herein, including the figures and sequence listing. In exemplary embodiments, the STEAP1 ABD is a STEAP1 ABD depicted in FIG. 22 or 39. In exemplary embodiments, the STEAP1 ABD comprises a VH and VL pair selected from those depicted in FIGS. 22 and 39.

In exemplary embodiments, the STEAP1 ABD comprises a VH selected from those depicted in FIG. 31 and a VL selected from those depicted in FIG. 32.

In addition to the parental STEAP1 variable heavy and variable light domains disclosed herein, provided herein are STEAP1 ABDs that include a variable heavy domain and/or a variable light domain that are variants of a STEAP1 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a STEAP1 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a STEAP1 ABD depicted in FIG. 22 or 39. In some embodiments, the changes are in a VH domain depicted in FIG. 22 or FIG. 31. In some embodiments, the changes are in a VL domain depicted in FIG. 22 or FIG. 32. In some embodiments, the changes are in a VH and VL domain depicted in FIG. 22. In some embodiments, the changes are in a VH depicted in FIG. 31 and a VL depicted in FIG. 32. In some embodiments, the one or more amino acid changes are in the VH and/or VL framework regions (FR1, FR2, FR3, and/or FR4). In some embodiments, the one or more amino acid change(s) are in one or more of vhCDR1-3 and/or vlCDR1-3. In some embodiments, the STEAP1 variant includes the vhCDR1-3 and/or vlCDR1-3 of any of the STEAP1 ABDs in FIGS. 22 and 39. In some embodiments, the STEAP1 variant includes the vhCDR1-3 of any of the VH domains in FIG. 31 and the vlCDR1-3 of any of the VL domains in FIG. 32. In certain embodiments, the STEAP1 ABD is capable of binding to STEAP1 , as measured at least one of a Biacore, surface plasmon resonance (SPR), flow cytometry, and/or BLI (biolayer interferometry, e.g., Octet assay) assay, with the latter finding particular use in many embodiments. In particular embodiments, the STEAP1 ABD is capable of binding human STEAP1 antigen (FIG. 2).

In one embodiment, the variant VH and/or VL domain is at least 90, 95, 97, 98 or 99% identical to the VH and/or VL of a STEAP1 ABD as described herein, including the figures and sequence listing. In exemplary embodiments, the variant VH and/or VL domain is at least 90, 95, 97, 98 or 99% identical to the VH and/or VL of a STEAP1 ABD depicted in FIG. 22. In some embodiments, the STEAP1 ABD includes a VH that is at least 90, 95, 97, 98 or 99% identical to a VH domain depicted in FIG. 22 or FIG. 31. In some embodiments, the STEAP1 ABD includes a VL that is at least 90, 95, 97, 98 or 99% identical to a VL domain depicted in FIG. 22 or FIG. 32. In some embodiments, the STEAP1 ABD includes a VH and a VL that is at least 90, 95, 97, 98 or 99% identical to a VH domain and a VL domain depicted in FIG. 22. In some embodiments, the STEAP1 ABD includes a VH and a VL that is at least 90, 95, 97, 98 or 99% identical to a VH domain depicted in FIG. 31 and a VL domain depicted in FIG. 32. In some embodiments, the STEAP1 ABD includes the 6 CDRs (vhCDR1-3 and vlCDR1-3) of any of the STEAP1 ABDs of FIG. 22. In some embodiments, the STEAP1 ABD includes the vhCDRT-3 of any of the VH domains depicted in FIG. 31 and the vlCDR1-3 of any of the VL domains depicted in FIG. 32. In certain embodiments, the STEAP1 is capable of binding to the STEAP1 , as measured by at least one of a Biacore, surface plasmon resonance (SPR), flow cytometry, and/or BLI (biolayer interferometry, e.g., Octet assay) assay, with the latter finding particular use in many embodiments. In particular embodiments, the STEAP1 ABD is capable of binding human STEAP1 antigen (FIG. 2).

The anti-CD28 x anti-STEAP1 antibodies provided herein include at least one STEAP1 binding domain. Subject antibodies that include such STEAP1 antigen binding domains (e.g., anti-STEAP1 x anti-CD3 bispecific antibodies) advantageously target cells that express high levels of STEAP1 over those that express levels of STEAP1 (e.g., normal cells).

In some embodiments, the STEAP1 ABD of the anti-CD28 x anti-STEAP1 antibody includes a set of 6 CDRs as depicted in the sequence listing and FIG. 22 or FIG. 31 and FIG. 32, either as the CDRs are underlined or, in the case where a different numbering scheme is used as described herein and as shown in Table 2, as the CDRs that are identified using other alignments within the variable heavy (VH) domain sequence of those depicted in FIG. 22 and FIG. 31, the variable light domain (VL) sequences of those depicted in FIG. 22 and FIG. 32, and the sequence listing (see Table 2).

In exemplary embodiments, the STEAP1 ABD of the anti-CD28 x anti-STEAP1 antibody includes the variable heavy (VH) domain and variable light (VL) domain of any one of the STEAP1 ABDs described herein, including the figures and sequence listing.

In exemplary embodiments, the STEAP1 ABD of the anti-CD28 x anti-STEAP1 antibody is a STEAP1 ABD depicted in FIG. 22 or 39. In exemplary embodiments, the STEAP1 ABD of the anti-CD28 x anti-STEAP1 antibody includes any one of the variable heavy (VH) domains depicted in FIG. 31 and any one of the variable light (VL) domains depicted in FIG. 32.

In addition to the parental STEAP1 variable heavy and variable light domains disclosed herein, provided herein are STEAP1 ABDs that include a variable heavy domain and/or a variable light domain that are variants of a STEAP1 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a STEAP1 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a STEAP1 ABD depicted in FIG. 22 or 39. In exemplary embodiments, the variant VH domain and/or VL domain has from 1, 2, 3, 4, 5, 6, 7, 8, 9 or 10 amino acid changes from a VH domain depicted in FIG. 31 and/or a VL domain depicted in FIG. 32. In some embodiments, the changes are in a VH domain depicted in FIG. 22 or FIG. 31. In some embodiments, the changes are in a VL domain depicted in FIG. 22 or FIG. 32. In some embodiments, the changes are in a VH and VL domain depicted in FIG. 22. In some embodiments, the changes are in a VH domain depicted in FIG. 31 and a VL domain depicted in FIG. 32. In some embodiments, the one or more amino acid changes are in the VH and/or VL framework regions (FR1, FR2, FR3, and/or FR4). In some embodiments, the one or more amino acid change(s) are in one or more of vhCDR1-3 and/or vlCDR1-3. In some embodiments, the STEAP1 variant includes the vhCDR1-3 and/or vlCDR1-3 of any of the STEAP1 ABDs in FIG. 22 or 39. In some embodiments, the STEAP1 variant includes the vhCDR1-3 of any of the VH domains depicted in FIG. 31 and the vlCDR1-3 of any of the VL domains depicted in FIG. 32. In certain embodiments, the STEAP1 ABD of the anti-CD28 x anti-STEAP1 antibody is capable of binding to STEAP1 , as measured at least one of a Biacore, surface plasmon resonance (SPR), flow cytometry, and/or BLI (biolayer interferometry, e.g., Octet assay) assay, with the latter finding particular use in many embodiments. In particular embodiments, the STEAP1 ABD is capable of binding human STEAP1 antigen (FIG. 2).

In one embodiment, the variant VH and/or VL domain is at least 90, 95, 97, 98 or 99% identical to the VH and/or VL of a STEAP1 ABD as described herein, including the figures and sequence listing. In exemplary embodiments, the variant VH and/or VL domain is at least 90, 95, 97, 98 or 99% identical to the VH and/or VL of a STEAP1 ABD depicted in FIG. 22 or 39. In exemplary embodiments, the variant VH domain and/or VL domain is at least 90, 95, 97, 98 or 99% identical to the VH domain depicted in FIG. 31 and/or the VL domain depicted in FIG. 32. In some embodiments, the STEAP1 ABD includes a VH that is at least 90, 95, 97, 98 or 99% identical to VH domain depicted in FIG. 22 or FIG. 31. In some embodiments, the STEAP1 ABD includes a VL that is at least 90, 95, 97, 98 or 99% identical to VL domain depicted in FIG. 22 or FIG. 32. In some embodiments, the STEAP1 ABD includes a VH and a VL that is at least 90, 95, 97, 98 or 99% identical to a VH domain and a VL domain depicted in FIG. 22 or 39. In some embodiments, the STEAP1 ABD includes a VH and a VL that is at least 90, 95, 97, 98 or 99% identical to a VH domain depicted in FIG. 31 and a VL domain depicted in FIG. 32. In some embodiments, the STEAP1 ABD includes the 6 CDRs (vhCDR1-3 and vlCDR1-3) of any of the STEAP1 ABDs of FIG. 22. In some embodiments, the STEAP1 ABD includes the vhCDR1-3 of any of the VH domains depicted in FIG. 31 and the vlCDR1-3 of any of the VL domains depicted in FIG. 32. In certain embodiments, the STEAP1 ABD of the anti-CD28 x anti-STEAP1 antibody is capable of binding to the STEAP1 , as measured by at least one of a Biacore, surface plasmon resonance (SPR), flow cytometry, and/or BLI (biolayer interferometry, e.g., Octet assay) assay, with the latter finding particular use in many embodiments. In particular embodiments, the STEAP1 ABD is capable of binding human STEAP1 antigen (FIG. 2).

In some embodiments, the anti-CD28 x anti-STEAP1 antibody is a bivalent antibody (e.g., 1+1 Fab-scFv-Fc format antibody) that includes one STEAP1 binding domain.

In other embodiments, the anti-CD28 x anti-STEAP1 antibody is a trivalent antibody (e.g., 2+1 mAb-scFv, 2+1 Fab2.scFv-Fc, and 2+1 Fab2-Fc x scFv-Fc format antibodies) that includes two STEAP1 binding domains.

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10⁻²⁰ amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-CD28 x anti-STEAP1 antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers.

As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below. 1. Skew Variants

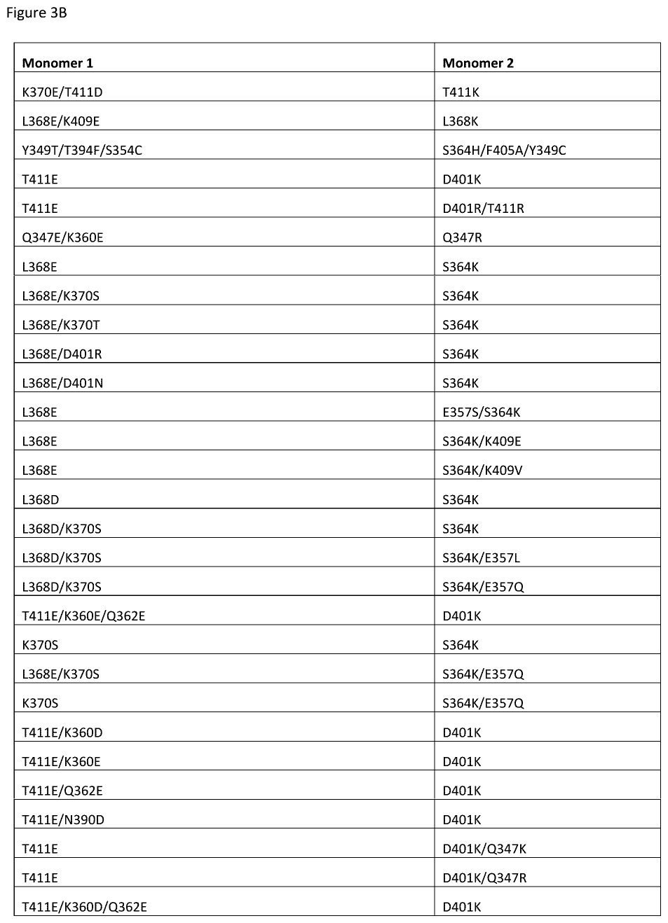

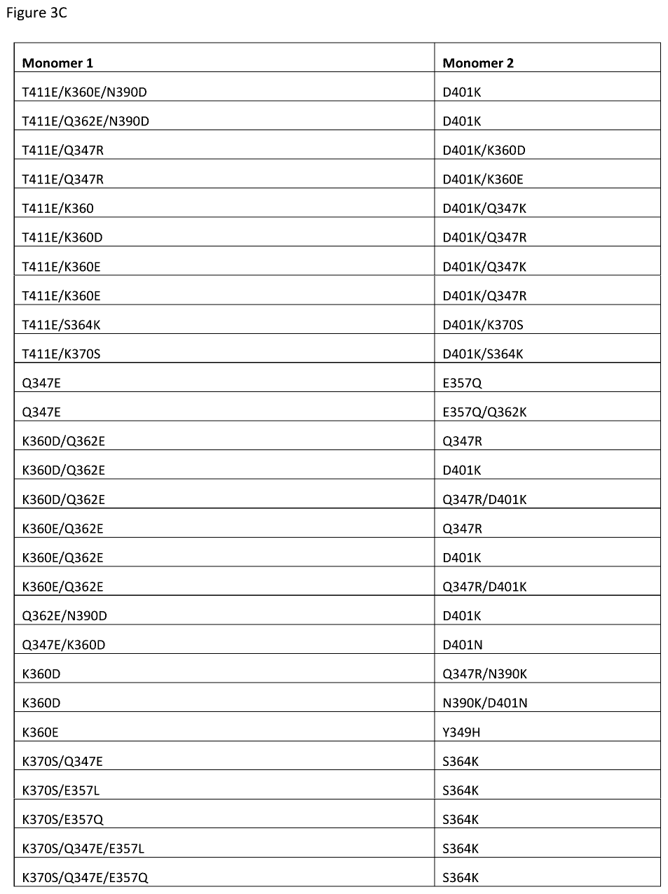

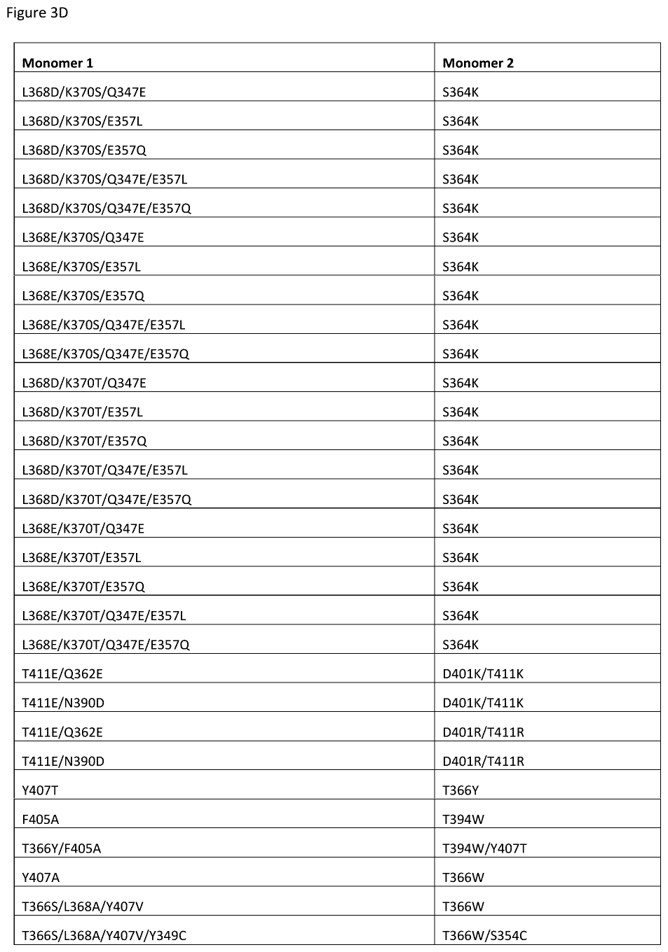

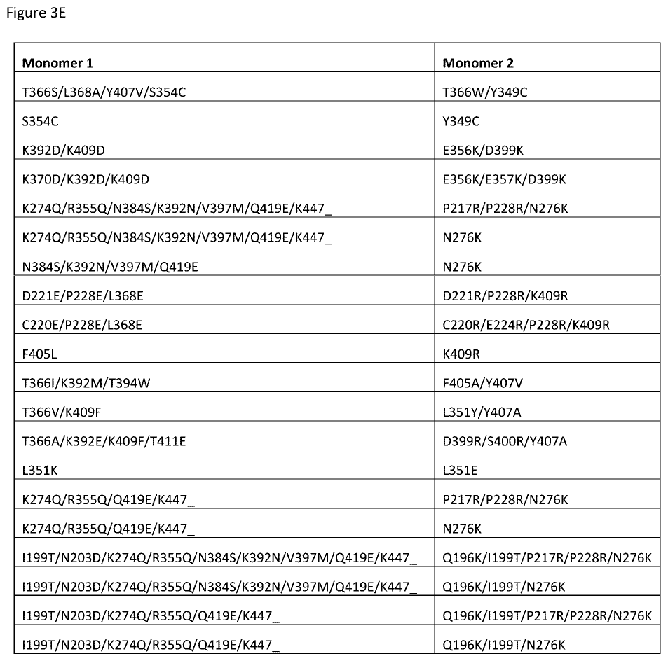

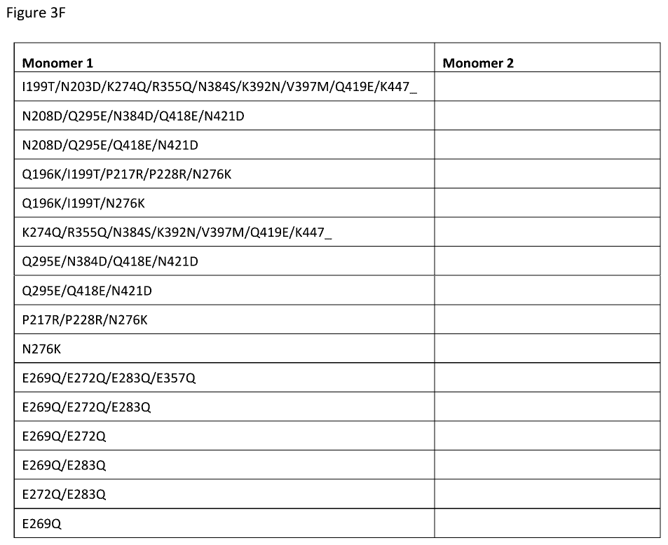

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3 and 8.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A- monomer B” pairs that rely on “knobs and holes.” In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants.” These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3 and 8. Such “skew” variants include, but are not limited to: S364K/E357Q: L368D/K370S; L368D/K370S: S364K; L368E/K370S: S364K; T411T/E360E/Q362E: D401K; L368D/K370S: S364K/E357L; K370S: S364K/E357Q (EU numbering).

In exemplary embodiments, the heterodimeric antibody includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) “skew” variant amino acid substitution set (EU numbering). In an exemplary embodiment, the heterodimeric antibody includes a “S364K/E357Q: L368D/K370S” amino acid substitution set. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies. 2. Purification Variants

In some embodiments, the heterodimeric antibody includes purification variants that advantageously allow for the separation of heterodimeric proteins (e.g., anti-CD28 x anti-STEAP1 bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins.

Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants.

Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

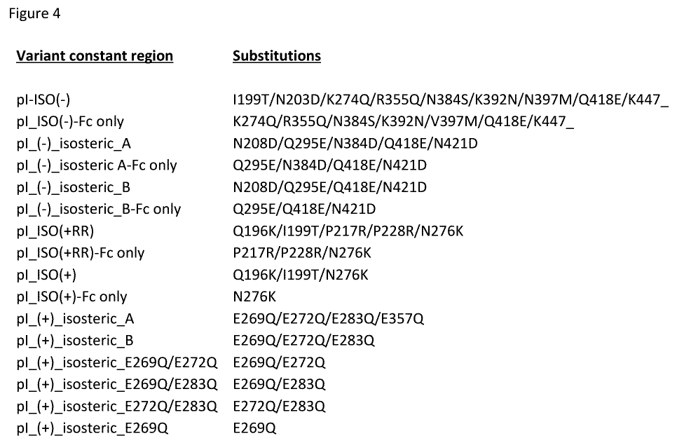

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIGS. 3 and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab2-scFv-Fc, 1+1 CLC and 2+1 CLC formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 3 and 4, and FIG. 30 of US Publ. App. No. 2016/0355608, all of which are herein incorporated by reference in its entirety and specifically for the disclosure of pI variants. Preferred combinations of pI variants are shown in FIGS. 3 and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄(SEQ ID NO: 24). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 8 and the other monomer has a charged linker (either in the form of a charged scFv linker because that monomer comprises an scFv or a charged domain linker, as the format dictates, which can be selected from those depicted in FIG. 6).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-CD28 x anti-STEAP1 antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared. 5. pI Variants that also confer better FcRn in vivo binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood.

In mice, Dall′ Acqua et al. showed that Fc mutants with increased FcRn binding at pH 6 and pH 7.4 actually had reduced serum concentrations and the same half-life as wild-type Fc (Dall′ Acqua et al. 2002, J. Immunol. 169:5171-5180, entirely incorporated by reference).

The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. Nos. 8,188,321 (particularly FIG. 41) and 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 2591, 428L/434S, 2591/308F, 4361/428L, 4361 or V/434S, 436V/428L, 259I/308F/428L, and M252Y/S254T/T256E. Such modification may be included in one or both Fc domains of the subject antibody.

### 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred aspects utilizing ablation variants selected from the group consisting of. L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition.” In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 mAb-Fc, 2+1 Fab2-scFv-Fc, and 2+1 Fab2-Fc x scFv-Fc format antibodies are included in FIG. 8. In some embodiments, the heterodimeric antibody includes a combination of variants as depicted in FIG. 8.

### G. Useful Antibody Formats

As will be appreciated by those in the art and discussed more fully below, the heterodimeric bispecific antibodies provided herein can take on several different configurations as generally depicted in FIG. 14.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain. As is outlined herein, when CD28 is one of the target antigens, it is preferable that the CD28 is bound only monovalently.

The present invention utilizes CD28 binding domains in combination with STEAP1 binding domains. As will be appreciated by those in the art, any collection of anti-CD28 CDRs, anti-CD28 variable light and variable heavy domains, Fabs and scFvs as depicted in any of the Figures (see particularly FIGS. 15, 18, 21 and 65) or variants thereof can be used. Similarly, any of the STEAP1 antigen binding domains can be used, whether CDRs, variable light and variable heavy domains, Fabs and scFvs as depicted in any of the Figures (see e.g., FIGS. 22, 31, and 32) or variants thereof can be used, optionally and independently combined in any combination.

1. 1+1 Fab-scFv-Fc format

One heterodimeric antibody format that finds particular use in subject anti-CD28 x anti-STEAP1 antibodies provided herein is the “1+1 Fab-scFv-Fc” or “bottle opener” format as shown in FIG. 14A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv,” as defined below) and a second Fc domain.

The scFv includes a second variable heavy domain (VH2) and a second variable light domain (VL2), wherein the VH2 is attached to the VL2 using an scFv linker that can be charged (see, e.g., FIG. 6). The scFv is attached to the heavy chain using a domain linker (see, e.g., FIG. 7). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a CD28 binding domain and the other binding domain is a STEAP1 binding domain. In some embodiments where the 1+1 Fab-scFv-Fc, it is the scFv that binds to the CD28, and the Fab that binds STEAP1 . Exemplary anti-CD28 x anti-STEAP1 bispecific antibodies in the 1+1 Fab-scFv-Fc format are depicted in FIG. 24.

In some embodiments, the first and second Fc domains of the 1+1 Fab-scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236del/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody includes a combination of amino acid modifications as depicted in FIG. 8. In such embodiments, the CH1-hinge-CH2-CH3 of the first monomer comprises amino acid variants L368D/K370S/N208D/Q295E/N384D/Q418E/N421D/E233P/L234V/L235A/G236del/S267 K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 6). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody, the first Fc domain includes heterodimerization skew variants L368D/K370S and the second Fc domain includes heterodimerization skew variants S364K/E357Q; each of the first and second Fc domains include ablation variants E233P/L234V/L235A/G236del/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker (SEQ ID NO: 24). In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in FIG. 6).

Any suitable CD28 binding domain can be included in subject 1+1 Fab-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). Additional VH and VL sequences of exemplary CD28 binding domains that can be used in the subject 1+1 Fab-scFv-Fc format antibody are depicted in FIGS. 15, 16, 17, and 82.

In some embodiments of the 1+1 Fab-scFv-Fc format, the anti-CD28 ABD has a VH and VL domain selected from the following:(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of a VL of the CD28 binding domains depicted in FIGS. 15, 18, 21 and 65 or a variant thereof; or(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIGS. 15, 17 or 82 or a variant thereof.

In some embodiments of the 1+1 Fab-scFv-Fc format, the anti-CD28 ABD has a VH and VL domain selected from the following: (i) a VH having an amino acid sequence of a VH of any of the CD28 binding domains depicted in FIGS. 15, 18, 21 and 65 or a variant thereof; and (ii) a VL having an amino acid sequence of a VL of the CD28 binding domain depicted in FIGS. 15, 18, 21 and 65 or a variant thereof; or (i) a VH having an amino acid sequence of a VH depicted in FIG. 15 or 16 or a variant thereof; and (ii) a VL having an amino acid sequence of the VL of a VL depicted in FIGS. 15, 17 or 82 or a variant thereof.

Any suitable STEAP1 binding domain can be included in subject 1+1 Fab-scFv-Fc format antibody, including any of the STEAP1 binding domains provided herein. In some embodiments, the STEAP1 binding domain is one of the following STEAP1 binding domains or a variant thereof. H1 and L1, H1.1 and L1, H1.2 and L1, H1.3 and L1, H1.4 and L1, H1.5 and L1, H1.6 and L1, H1.7 and L1, H1.8 and L1, H1.9 and L1, H1.10 and L1, H1.II and L1, H1.12 and L1, H1.13 and L1, H1.14 and L1, H1.15 and L1, H1.16 and L1, H1.17 and L1, H1.18 and L1, H1.19 and L1, H1.20 and L1, H1.21 and L1, H1.22 and L1, H1.23 and L1, H1.24 and L1, H1.25 and L1, H1.26 and L1, H1.27 and L1, H1.28 and L1, H1.29 and L1, H1.30 and L1, H1.31 and L1, H1.32 and L1, H1.33 and L1, H1.34 and L1, H1.35 and L1, H1.36 and L1, H1.37 and L1, H1.38 and L1, H1 and L1.1, H1 and L1.2, H1 and L1.3, H0 and L0, H1.21 and L1.3, H1.34 and L1.3, H1.39 and L1, H1.40 and L1, H1.4I and L1, H1.42 and L1, H1.43 and L1, H1.44 and L1, H1.45 and L1, H1.46 and L1, H1.47 and L1, H1.48 and L1, H1.49 and L1, H1.50 and L1, H1.51 and L1, H1.52 and L1, H1.53 and L1, H1.54 and L1, H1.55 and L1, H1.56 and L1, H1.57 and L1, H1.58 and L1, H1.59 and L1, H1.60 and L1, H1.61 and L1, H1.62 and L1, H1.63 and L1, H1.64 and L1, H1.65 and L1, H1.66 and L1, H1.67 and L1, H1.68 and L1, H1.69 and L1, H1.70 and L1, H1.71 and L1, H1.72 and L1, H1.73 and L1, H1.74 and L1, H1.75 and L1, H1.76 and L1, H1.77 and L1, H1.78 and L1, H1.79 and L1, H1.80 and L1, H1.81 and L1, H1.82 and L1, H1.83 and L1, H1.84 and L1, H1.85 and L1, H1.86 and L1, H1.87 and L1, H1.88, and L1, H1.89 and L1, H1.90 and L1, H1.91 and L1, H1.92 and L1, H1.93 and L1, H1.94 and L1, H1.95 and L1, H1.96 and L1, H1.97 and L1, H1.98 and L1, H1.99 and L1, H1.100 and L1, H1.101 and L1, H1.102 and L1, H1.103 and L1, H1.104 and L1, H1.105 and L1, H1.106 and L1, H1.107 and L1, H1.108 and L1, H1.109 and L1, H1.110 and L1, H1.III and L1, H1.112 and L1, H1.113 and L1, H1.114 and L1, H1.115 and L1, H1.116 and L1, H1.117 and L1, H1.118 and L1, H1.119 and L1, H1.120 and L1, H1.121 and L1, H1.122 and L1, H1.123 and L1, H1.124 and L1, H1.125 and L1, H1.126 and L1, H1.127 and L1, H1.128 and L1, H1.129 and L1, H1.130 and L1, H1.131 and L1, H1.132 and L1, H1.133 and L1, H1.134 and L1, H1.135 and L1, H1.136 and L1, H1.137 and L1, H1.138 and L1, H1.139 and L1, H1.140 and L1, H1.141 and L1, H1.142 and L1, H1.143 and L1, H1.144 and L1, H1.145 and L1, H1.146 and L1, H1.147 and L1, H1.148 and L1, H1.149 and L1, H1.150 and L1, H1.151 and L1, H1.152 and L1, H1.153 and L1, H1.154 and L1, H1.155 and L1, H1.156 and L1, H1.157 and L1, H1.158 and L1, H1.159 and L1, H1.160 and L1, H1.161 and L1, H1.162 and L1, H1.163 and L1, H1.164 and L1, H1.165 and L1, H1.166 and L1, H1.167 and L1, H1.168 and L1, H1.169 and L1, H1.170 and L1, H1.171 and L1, H1.172 and L1, H1.173 and L1, H1.174 and L1, H1.175 and L1, H1.176 and L1, H1.177 and L1, H1.178 and L1, H1.179 and L1, H1.180 and L1, H1.181 and L1, H1.182 and L1, H1.183 and L1, H1.184 and L1, H1.185 and L1, H1.186 and L1, H1.187 and L1, H1.188 and L1, H1.189 and L1, H1 and L1.4, H1 and L1.5, H1 and L1.6, H1 and L1.7, H1 and L1.8, H1 and L1.9, H1 and L1.10, H1 and L1.11, H1 and L1.12, H1 and L1.13, H1 and L1.14, H1 and L1.15, H1 and L1.16, H1 and L1.17, H1 and L1.18, H1 and L1.19, H1 and L1.20, H1 and L1.21, H1 and L1.22, H1 and L1.23, H1 and L1.24, H1 and L1.25, H1 and L1.26, H1 and L1.27, H1 and L1.28, H1 and L1.29, H1 and L1.30, H1 and L1.31, H1 and L1.32, H1 and L1.33, H1 and L1.34, H1 and L1.35, H1 and L1.36, H1 and L1.37, H1 and L1.38, H1 and L1.39, H1 and L1.40, H1 and L1.41, H1 and L1.42, H1 and L1.43, H1 and L1.44, H1 and L1.45, H1 and L1.46, H1 and L1.47, H1 and L1.48, H1 and L1.49, H1 and L1.50, H1 and L1.51, H1 and L1.52, H1 and L1.53, H1 and L1.54, H1 and L1.55, H1 and L1.56, H1 and L1.57, H1 and L1.58, H1 and L1.59, H1 and L1.60, H1 and L1.61, H1 and L1.62, H1 and L1.63, H1 and L1.64, H1 and L1.65, H1 and L1.66, H1 and L1.67, H1 and L1.68, H1 and L1.69, H1 and L1.70, H1 and L1.71, H1 and L1.72, H1 and L1.73, H1 and L1.74, H1 and L1.75, H1 and L1.76, H1 and L1.77, H1 and L1.78, H1 and L1.79, H1 and L1.80, H1 and L1.81, H1 and L1.82, H1 and L1.83, H1 and L1.84, H1 and L1.85, H1.42 and L1.47, H1.42 and L1.56, H1.190 and L1.3, H1.191 and L1.3, H1.192 and L1.3, H1.195 and L1.3, H1.196 and L1.3, H1.197 and L1.3, H1.198 and L1.3, H1.199 and L1.3, H1.200 and L1.3, H1.201 and L1.3, H1.202 and L1.3, H1.203 and L1.3, H1.204 and L1.3, H1.205 and L1.3, H1.206 and L1.3, H1.207 and L1.3, H1.208 and L1.3, H1.209 and L1.3, H1.210 and L1.3, H1.211 and L1.3, H1.212 and L1.3, H1.213 and L1.3, H1.214 and L1.3, H1.215 and L1.3, H1.216 and L1.3, H1.217 and L1.3, H1.218 and L1.3, H1.219 and L1.3, H1.220 and L1.3, H1.221 and L1.3, H1.222 and L1.3, H1.223 and L1.3, H1.224 and L1.3, H1.225 and L1.3, H1.226 and L1.3, H1.227 and L1.3, H1.228 and L1.3, H1.21 and L1.92, H1.21 and L1.93, H1.21 and L1.94, H1.21 and L1.95, H1.21 and L1.96, H1.21 and L1.97, H1.21 and L1.98, H1.21 and L1.99, H1.21 and L1.100, H1.21 and L1.101, H1.21 and L1.102, H1.21 and L1.103, H1.21 and L1.104, H1.21 and L1.105, H1.21 and L1.106, H1.21 and L1.107, H1.21 and L1.108, H1.21 and L1.109, H1.21 and L1.110, H1.21 and L1.111, H1.21 and L1.112, H1.21 and L1.113, H1.21 and L1.114, H1.21 and L1.115, H1.21 and L1.116, H1.21 and L1.117, H1.21 and L1.118, H1.21 and L1.119, H1.195 and L1.92, H1.195 and L1.94, H1.195 and L1.95, H1.195 and L1.104, H1.195 and L1.105, H1.195 and L1.109, H1.196 and L1.92, H1.196 and L1.94, H1.196 and L1.95, H1.196 and L1.104, H1.196 and L1.105, H1.196 and L1.109, H1.205 and L1.92, H1.205 and L1.94, H1.205 and L1.95, H1.205 and L1.104, H1.205 and L1.105, H1.205 and L1.109, H1.193 and L1.91, H1.194 and L1.86, H1.194 and L1.87, H1.194 and L1.88, H1.194 and L1.89, or H1.194 and L1.90, or H1.2320 and L1.100, or H1.230 and L1.115 (FIGS. 22, 31, 32 and 39). Additional VH and VL sequences of exemplary STEAP1 binding domains that can be used in the subject 1+1 Fab-scFv-Fc format antibody are depicted in FIGS. 22, 31, and 32.

In some embodiments of the 1+1 Fab-scFv-Fc format, the STEAP1 ABD has a VH and VL domain selected from the following: (i) a VH comprising a vhCDR1, a vhCDR2, and a vhCDR3 having an amino acid sequence of a vhCDR1, a vhCDR2, and a vhCDR3, respectively, of a VH of any of the STEAP1 binding domains depicted in FIG. 22, or a variant thereof; and (ii) a VL comprising a vlCDR1, a vlCDR2, and a vlCDR3 having an amino acid sequence of a vlCDR1, a vlCDR2, and a vlCDR3, respectively, of a VL of any of the STEAP1 binding domains depicted in FIG. 22, or a variant thereof; or (i) a VH comprising a vhCDR1, a vhCDR2, and a vhCDR3 having an amino acid sequence of a vhCDR1, a vhCDR2, and a vhCDR3, respectively, of a VH depicted in FIG. 22 or FIG. 31 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 22 or FIG. 32 or a variant thereof.

In some embodiments of the 1+1 Fab-scFv-Fc format, the STEAP1 ABD has a VH and VL domain selected from the following: (i) a VH having an amino acid sequence of any of the VH of any of the STEAP1 binding domains depicted in FIG. 22 or a variant thereof; and (ii) a VL having an amino acid sequence of a VL of any of the STEAP1 binding domains depicted in FIG. 22; or (i) a VH having an amino acid sequence of a VH depicted in FIG. 22 or FIG. 31 or a variant thereof; and (ii) a VL having an amino acid sequence of a VL depicted in FIG. 22 or FIG. 32 or a variant thereof.

FIG. 9 shows some exemplary Fc domain sequences that are useful in the 1 +1 Fab-scFv-Fc format antibodies. The “monomer 1” sequences depicted in FIG. 9 typically refer to the Fc domain of the “Fab-Fc heavy chain” and the “monomer 2” sequences refer to the Fc domain of the “scFv-Fc heavy chain.” In addition, FIGS. 11 and 12 provide exemplary CH1-hinge domains, CH1 domains, and hinge domains that can be included in the first or second monomer of the 1+1 Fab-scFv-Fc format. Further, FIG. 13 provides useful CL sequences that can be used with this format. 2. 2+1 mAb-scFv Format

One heterodimeric antibody format that finds particular use in the subject bispecific anti-CD28 x anti-STEAP1 antibodies is the 2+1 mAb-scFv format shown in FIG. 14E. This antibody format includes three antigen binding domains: two Fab portions and an scFv that is attached to the C-terminal of one of the heavy chains. In some embodiments of this format, the Fab portions each bind STEAP1 , in this case, human STEAP1 and the “extra” scFv domain binds CD28. That is, this mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In embodiments, this format includes two identical light chains (VL1-CL). In this format, the VHls are each a first variable heavy (VH) domain, the VL1s are each a first variable light (VL) domain, the VH2 is a second variable heavy domain, and the VL2 is a second variable light domain. In some embodiments, the format includes two identical common light chains (each VL1-CL), wherein each of the common light chains associates with one of the VH1-CH1 of the first and second monomers to form two identical Fabs (i.e., first antigen binding domains). In some embodiments, the scFv is the second antigen binding domain. In some embodiments, the first ABDs bind to human STEAP1 , and the second ABD binds human CD28.

In some embodiments, the first and second Fc domains of the 2+1 mAb-scFv format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fe domains include ablation variants (including those shown in FIG. 5). In some embodiments, each of the first and second variant Fc domains include ablation variants E233P/L234V/L235A/G236del/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 6). In some embodiments, the 2+1 mAb-scFv format antibody provided herein includes FcRn variants M428L/N434S, wherein numbering is according to EU numbering.

In exemplary embodiments the 2+1 mAb-scFv format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236del/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker (SEQ ID NO: 24). In some embodiments, 2+1 mAb-scFv format antibody provided herein includes FcRn variants M428L/N434S, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind STEAP1 . Any suitable STEAP1 binding domain and CD28 binding domain can be included in the 2+1 mAb-scFv format antibody, including any of the STEAP1 binding domains and CD28 binding domains and related VHs and VLs provided herein or a variant thereof (see, e.g., FIGS. 15, 18, and 21).

Any suitable CD28 binding domain can be included in subject 2+1 mAb-scFv format antibody, including any of the CD28 binding domains provided herein. In some embodiments, the CD28 binding domain is one of the following CD28 binding domains or a variant thereof: IA7[CD28]_H1L1, IA7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). Additional VH and VL sequences of exemplary CD28 binding domains that can be used in the subject 2+1 mAb-scFv format antibody are depicted in FIGS. 15, 16, 17, and 82.

In some embodiments of the 2+1 mAb-scFv format, the anti-CD28 ABD has a VH and VL domain selected from the following:(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIGS. 15, 17 or 82 or a variant thereof.

In some embodiments of the 2+1 mAb-scFv format, the anti-CD28 ABD has a VH and VL domain selected from the following:(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or(i) a VH having an amino acid sequence of a VH depicted in FIG. 15 or 16 or a variant thereof; and (ii) a VL having an amino acid sequence of a VL depicted in FIGS. 15, 17, or 82 or a variant thereof.

Any suitable STEAP1 binding domain can be included in subject 2+1 mAb-scFv format, including any of the STEAP1 binding domains provided herein. In some embodiments, the STEAP1 binding domain is one of the following STEAP1 binding domains or a variant thereof. H1 and L1, H1.1 and L1, H1.2 and L1, H1.3 and L1, H1.4 and L1, H1.5 and L1, H1.6 and L1, H1.7 and L1, H1.8 and L1, H1.9 and L1, H1.10 and L1, H1.II and L1, H1.12 and L1, H1.13 and L1, H1.14 and L1, H1.15 and L1, H1.16 and L1, H1.17 and L1, H1.18 and L1, H1.19 and L1, H1.20 and L1, H1.21 and L1, H1.22 and L1, H1.23 and L1, H1.24 and L1, H1.25 and L1, H1.26 and L1, H1.27 and L1, H1.28 and L1, H1.29 and L1, H1.30 and L1, H1.31 and L1, H1.32 and L1, H1.33 and L1, H1.34 and L1, H1.35 and L1, H1.36 and L1, H1.37 and L1, H1.38 and L1, H1 and L1.1, H1 and L1.2, H1 and L1.3, H0 and L0, H1.21 and L1.3, H1.34 and L1.3, H1.39 and L1, H1.40 and L1, H1.4I and L1, H1.42 and L1, H1.43 and L1, H1.44 and L1, H1.45 and L1, H1.46 and L1, H1.47 and L1, H1.48 and L1, H1.49 and L1, H1.50 and L1, H1.51 and L1, H1.52 and L1, H1.53 and L1, H1.54 and L1, H1.55 and L1, H1.56 and L1, H1.57 and L1, H1.58 and L1, H1.59 and L1, H1.60 and L1, H1.61 and L1, H1.62 and L1, H1.63 and L1, H1.64 and L1, H1.65 and L1, H1.66 and L1, H1.67 and L1, H1.68 and L1, H1.69 and L1, H1.70 and L1, H1.71 and L1, H1.72 and L1, H1.73 and L1, H1.74 and L1, H1.75 and L1, H1.76 and L1, H1.77 and L1, H1.78 and L1, H1.79 and L1, H1.80 and L1, H1.81 and L1, H1.82 and L1, H1.83 and L1, H1.84 and L1, H1.85 and L1, H1.86 and L1, H1.87 and L1, H1.88, and L1, H1.89 and L1, H1.90 and L1, H1.91 and L1, H1.92 and L1, H1.93 and L1, H1.94 and L1, H1.95 and L1, H1.96 and L1, H1.97 and L1, H1.98 and L1, H1.99 and L1, H1.100 and L1, H1.101 and L1, H1.102 and L1, H1.103 and L1, H1.104 and L1, H1.105 and L1, H1.106 and L1, H1.107 and L1, H1.108 and L1, H1.109 and L1, H1.110 and L1, H1.111 and L1, H1.112 and L1, H1.113 and L1, H1.114 and L1, H1.115 and L1, H1.116 and L1, H1.117 and L1, H1.118 and L1, H1.119 and L1, H1.120 and L1, H1.121 and L1, H1.122 and L1, H1.123 and L1, H1.124 and L1, H1.125 and L1, H1.126 and L1, H1.127 and L1, H1.128 and L1, H1.129 and L1, H1.130 and L1, H1.131 and L1, H1.132 and L1, H1.133 and L1, H1.134 and L1, H1.135 and L1, H1.136 and L1, H1.137 and L1, H1.138 and L1, H1.139 and L1, H1.140 and L1, H1.141 and L1, H1.142 and L1, H1.143 and L1, H1.144 and L1, H1.145 and L1, H1.146 and L1, H1.147 and L1, H1.148 and L1, H1.149 and L1, H1.150 and L1, H1.151 and L1, H1.152 and L1, H1.153 and L1, H1.154 and L1, H1.155 and L1, H1.156 and L1, H1.157 and L1, H1.158 and L1, H1.159 and L1, H1.160 and L1, H1.161 and L1, H1.162 and L1, H1.163 and L1, H1.164 and L1, H1.165 and L1, H1.166 and L1, H1.167 and L1, H1.168 and L1, H1.169 and L1, H1.170 and L1, H1.171 and L1, H1.172 and L1, H1.173 and L1, H1.174 and L1, H1.175 and L1, H1.176 and L1, H1.177 and L1, H1.178 and L1, H1.179 and L1, H1.180 and L1, H1.181 and L1, H1.182 and L1, H1.183 and L1, H1.184 and L1, H1.185 and L1, H1.186 and L1, H1.187 and L1, H1.188 and L1, H1.189 and L1, H1 and L1.4, H1 and L1.5, H1 and L1.6, H1 and L1.7, H1 and L1.8, H1 and L1.9, H1 and L1.10, H1 and L1.11, H1 and L1.12, H1 and L1.13, H1 and L1.14, H1 and L1.15, H1 and L1.16, H1 and L1.17, H1 and L1.18, H1 and L1.19, H1 and L1.20, H1 and L1.21, H1 and L1.22, H1 and L1.23, H1 and L1.24, H1 and L1.25, H1 and L1.26, H1 and L1.27, H1 and L1.28, H1 and L1.29, H1 and L1.30, H1 and L1.31, H1 and L1.32, H1 and L1.33, H1 and L1.34, H1 and L1.35, H1 and L1.36, H1 and L1.37, H1 and L1.38, H1 and L1.39, H1 and L1.40, H1 and L1.41, H1 and L1.42, H1 and L1.43, H1 and L1.44, H1 and L1.45, H1 and L1.46, H1 and L1.47, H1 and L1.48, H1 and L1.49, H1 and L1.50, H1 and L1.51, H1 and L1.52, H1 and L1.53, H1 and L1.54, H1 and L1.55, H1 and L1.56, H1 and L1.57, H1 and L1.58, H1 and L1.59, H1 and L1.60, H1 and L1.61, H1 and L1.62, H1 and L1.63, H1 and L1.64, H1 and L1.65, H1 and L1.66, H1 and L1.67, H1 and L1.68, H1 and L1.69, H1 and L1.70, H1 and L1.71, H1 and L1.72, H1 and L1.73, H1 and L1.74, H1 and L1.75, H1 and L1.76, H1 and L1.77, H1 and L1.78, H1 and L1.79, H1 and L1.80, H1 and L1.81, H1 and L1.82, H1 and L1.83, H1 and L1.84, H1 and L1.85, H1.42 and L1.47, H1.42 and L1.56, H1.190 and L1.3, H1.191 and L1.3, H1.192 and L1.3, H1.195 and L1.3, H1.196 and L1.3, H1.197 and L1.3, H1.198 and L1.3, H1.199 and L1.3, H1.200 and L1.3, H1.201 and L1.3, H1.202 and L1.3, H1.203 and L1.3, H1.204 and L1.3, H1.205 and L1.3, H1.206 and L1.3, H1.207 and L1.3, H1.208 and L1.3, H1.209 and L1.3, H1.210 and L1.3, H1.211 and L1.3, H1.212 and L1.3, H1.213 and L1.3, H1.214 and L1.3, H1.215 and L1.3, H1.216 and L1.3, H1.217 and L1.3, H1.218 and L1.3, H1.219 and L1.3, H1.220 and L1.3, H1.221 and L1.3, H1.222 and L1.3, H1.223 and L1.3, H1.224 and L1.3, H1.225 and L1.3, H1.226 and L1.3, H1.227 and L1.3, H1.228 and L1.3, H1.21 and L1.92, H1.21 and L1.93, H1.21 and L1.94, H1.21 and L1.95, H1.21 and L1.96, H1.21 and L1.97, H1.21 and L1.98, H1.21 and L1.99, H1.21 and L1.100, H1.21 and L1.101, H1.21 and L1.102, H1.21 and L1.103, H1.21 and L1.104, H1.21 and L1.105, H1.21 and L1.106, H1.21 and L1.107, H1.21 and L1.108, H1.21 and L1.109, H1.21 and L1.110, H1.21 and L1.111, H1.21 and L1.112, H1.21 and L1.113, H1.21 and L1.114, H1.21 and L1.115, H1.21 and L1.116, H1.21 and L1.117, H1.21 and L1.118, H1.21 and L1.119, H1.195 and L1.92, H1.195 and L1.94, H1.195 and L1.95, H1.195 and L1.104, H1.195 and L1.105, H1.195 and L1.109, H1.196 and L1.92, H1.196 and L1.94, H1.196 and L1.95, H1.196 and L1.104, H1.196 and L1.105, H1.196 and L1.109, H1.205 and L1.92, H1.205 and L1.94, H1.205 and L1.95, H1.205 and L1.104, H1.205 and L1.105, H1.205 and L1.109, H1.193 and L1.91, H1.194 and L1.86, H1.194 and L1.87, H1.194 and L1.88, H1.194 and L1.89, or H1.194 and L1.90, or H1.2320 and L1.100, or H1.230 and L1.115 (FIGS. 22, 31, 32 and 39). Additional VH and VL sequences of exemplary STEAP1 binding domains that can be used in the subject 2+1 mAb-scFv format antibody are depicted in FIGS. 22, 31, and 32.

In some embodiments of the 2+1 mAb-scFv format, the STEAP1 ABD has a VH and VL domain selected from the following: (i) a VH comprising a vhCDR1, a vhCDR2, and a vhCDR3 having an amino acid sequence of a vhCDR1, a vhCDR2, and a vhCDR3, respectively, of a VH of any of the STEAP1 binding domains depicted in FIG. 22, or a variant thereof; and (ii) a VL comprising a vlCDR1, a vlCDR2, and a vlCDR3 having an amino acid sequence of a vlCDR1, a vlCDR2, and a vlCDR3, respectively, of a VL of any of the STEAP1 binding domains depicted in FIG. 22, or a variant thereof; or (i) a VH comprising a vhCDR1, a vhCDR2, and a vhCDR3 having an amino acid sequence of a vhCDR1, a vhCDR2, and a vhCDR3, respectively, of a VH depicted in FIG. 22 or FIG. 31 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 22 or FIG. 32 or a variant thereof.

In some embodiments of the 2+1 mAb-scFv format, the STEAP1 ABD has a VH and VL domain selected from the following: (i) a VH having an amino acid sequence of any of the STEAP1 binding domains depicted in FIG. 22 or a variant thereof; and (ii) a VL having an amino acid sequence of a VL of any of the STEAP1 binding domains depicted in FIG. 22 or a variant thereof; or (i) a VH having an amino acid sequence of a VH depicted in FIG. 22 or FIG. 31 or a variant thereof; and (ii) a VL having an amino acid sequence of a VL depicted in FIG. 22 or FIG. 32 or a variant thereof.

FIG. 10 shows some exemplary Fc domain sequences that are useful in the 2+1 mAb-scFv format antibodies. In addition, FIGS. 11 and 12 provide exemplary CH1-hinge domains, CH1 domains, and hinge domains that can be included in the first or second monomer of the 2+1 mAb-scFv format. Further, FIG. 13 provides useful CL sequences that can be used with this format. 3. 2+1 Fab2-scFv-Fc format

One heterodimeric antibody format that finds particular use in subject anti-CD28 x anti-STEAP1 antibodies provided herein is the 2+1 Fab2-scFv-Fc format (also referred to as “central-scFv format”) shown in FIG. 14B. This antibody format includes three antigen binding domains: two Fab portions and an scFv that is inserted between the VH-CH1 and CH2-CH3 regions of one of the monomers. In some embodiments of this format, the Fab portions each bind STEAP1 and the “extra” scFv domain binds CD28. In some embodiments, the 2+1 Fab2-scFv-Fc format antibody is a trivalent antibody.

In some embodiments of the 2+1 Fab2-scFv-Fc format, a first monomer includes a standard heavy chain (i.e., VH1-CHT-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VHT-CHT-[optional linker]-VH2-scFv linker-VH2-[optional linker]-CH2-CH3, or the opposite orientation for the scFv, VH1-CHT-[optional linker]-VL2-scFv linker-VH2-[optional linker]-CH2-CH3). The optional linkers can be any suitable peptide linkers, including, for example, the domain linkers included in FIG. 7. This embodiment further utilizes a common light chain that includes a variable light domain (VL1) and a constant light domain (CL). In some embodiments, the format includes two identical common light chains (each VL1-CL), wherein each of the common light chains associates with one of the VH1-CHT of the first and second monomers to form two identical Fabs. In some embodiments, the identical Fabs each bind STEAP1 , and the scFv binds CD28.

As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fe domains include ablation variants (including those shown in FIG. 5). In some embodiments, each of the first and second variant Fc domains include ablation variants E233P/L234V/L235A/G236del/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-scFv-Fc format antibody provided herein includes a charged scFv linker (including those shown in FIG. 6). In some embodiments, the 2+1 Fab2-scFv-Fc format antibody provided herein includes FcRn variants M428L/N434S, wherein numbering is according to EU numbering.

In exemplary embodiments 2+1 Fab2-scFv-Fc format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236del/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO: 24). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-scFv-Fc format antibody is a CD28 binding domain and the VH1 of the first and second monomer and the VL1 of the common light chain each form binding domains that bind STEAP1 . Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: IA7[CD28]_H1L1, IA7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65).

In some embodiments of the 2+1 Fab2-scFv-Fc format, the anti-CD28 ABD has a VH and VL domain selected from the following:(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIGS. 15, 17, or 82 or a variant thereof.

In some embodiments of the 2+1 Fab2-scFv-Fc format, the anti-CD28 ABD has a VH and VL domain selected from the following:(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or(i) a VH having an amino acid sequence of a VH depicted in FIG. 15 or 16 or a variant thereof; and (ii) a VL having an amino acid sequence of a VL depicted in FIGS. 15, 17, or 82 or a variant thereof.

In some embodiments, the VH1 of the first and second monomer and the VL1 of the common light chain of the 2+1 Fab2-scFv-Fc format antibody each form a binding domain that binds STEAP1 . Any suitable STEAP1 binding domain can be included in subject 2+1 Fab2-scFv-Fc, including any of the STEAP1 binding domains provided herein. In some embodiments, the STEAP1 binding domain is one of the following STEAP1 binding domains or a variant thereof. H1 and L1, H1.1 and L1, H1.2 and L1, H1.3 and L1, H1.4 and L1, H1.5 and L1, H1.6 and L1, H1.7 and L1, H1.8 and L1, H1.9 and L1, H1.10 and L1, H1.II and L1, H1.12 and L1, H1.13 and L1, H1.14 and L1, H1.15 and L1, H1.16 and L1, H1.17 and L1, H1.18 and L1, H1.19 and L1, H1.20 and L1, H1.21 and L1, H1.22 and L1, H1.23 and L1, H1.24 and L1, H1.25 and L1, H1.26 and L1, H1.27 and L1, H1.28 and L1, H1.29 and L1, H1.30 and L1, H1.31 and L1, H1.32 and L1, H1.33 and L1, H1.34 and L1, H1.35 and L1, H1.36 and L1, H1.37 and L1, H1.38 and L1, H1 and L1.1, H1 and L1.2, H1 and L1.3, H0 and L0, H1.21 and L1.3, H1.34 and L1.3, H1.39 and L1, H1.40 and L1, H1.4I and L1, H1.42 and L1, H1.43 and L1, H1.44 and L1, H1.45 and L1, H1.46 and L1, H1.47 and L1, H1.48 and L1, H1.49 and L1, H1.50 and L1, H1.51 and L1, H1.52 and L1, H1.53 and L1, H1.54 and L1, H1.55 and L1, H1.56 and L1, H1.57 and L1, H1.58 and L1, H1.59 and L1, H1.60 and L1, H1.61 and L1, H1.62 and L1, H1.63 and L1, H1.64 and L1, H1.65 and L1, H1.66 and L1, H1.67 and L1, H1.68 and L1, H1.69 and L1, H1.70 and L1, H1.71 and L1, H1.72 and L1, H1.73 and L1, H1.74 and L1, H1.75 and L1, H1.76 and L1, H1.77 and L1, H1.78 and L1, H1.79 and L1, H1.80 and L1, H1.81 and L1, H1.82 and L1, H1.83 and L1, H1.84 and L1, H1.85 and L1, H1.86 and L1, H1.87 and L1, H1.88, and L1, H1.89 and L1, H1.90 and L1, H1.91 and L1, H1.92 and L1, H1.93 and L1, H1.94 and L1, H1.95 and L1, H1.96 and L1, H1.97 and L1, H1.98 and L1, H1.99 and L1, H1.100 and L1, H1.101 and L1, H1.102 and L1, H1.103 and L1, H1.104 and L1, H1.105 and L1, H1.106 and L1, H1.107 and L1, H1.108 and L1, H1.109 and L1, H1.110 and L1, H1.111 and L1, H1.112 and L1, H1.113 and L1, H1.114 and L1, H1.115 and L1, H1.116 and L1, H1.117 and L1, H1.118 and L1, H1.119 and L1, H1.120 and L1, H1.121 and L1, H1.122 and L1, H1.123 and L1, H1.124 and L1, H1.125 and L1, H1.126 and L1, H1.127 and L1, H1.128 and L1, H1.129 and L1, H1.130 and L1, H1.131 and L1, H1.132 and L1, H1.133 and L1, H1.134 and L1, H1.135 and L1, H1.136 and L1, H1.137 and L1, H1.138 and L1, H1.139 and L1, H1.140 and L1, H1.141 and L1, H1.142 and L1, H1.143 and L1, H1.144 and L1, H1.145 and L1, H1.146 and L1, H1.147 and L1, H1.148 and L1, H1.149 and L1, H1.150 and L1, H1.151 and L1, H1.152 and L1, H1.153 and L1, H1.154 and L1, H1.155 and L1, H1.156 and L1, H1.157 and L1, H1.158 and L1, H1.159 and L1, H1.160 and L1, H1.161 and L1, H1.162 and L1, H1.163 and L1, H1.164 and L1, H1.165 and L1, H1.166 and L1, H1.167 and L1, H1.168 and L1, H1.169 and L1, H1.170 and L1, H1.171 and L1, H1.172 and L1, H1.173 and L1, H1.174 and L1, H1.175 and L1, H1.176 and L1, H1.177 and L1, H1.178 and L1, H1.179 and L1, H1.180 and L1, H1.181 and L1, H1.182 and L1, H1.183 and L1, H1.184 and L1, H1.185 and L1, H1.186 and L1, H1.187 and L1, H1.188 and L1, H1.189 and L1, H1 and L1.4, H1 and L1.5, H1 and L1.6, H1 and L1.7, H1 and L1.8, H1 and L1.9, H1 and L1.10, H1 and L1.11, H1 and L1.12, H1 and L1.13, H1 and L1.14, H1 and L1.15, H1 and L1.16, H1 and L1.17, H1 and L1.18, H1 and L1.19, H1 and L1.20, H1 and L1.21, H1 and L1.22, H1 and L1.23, H1 and L1.24, H1 and L1.25, H1 and L1.26, H1 and L1.27, H1 and L1.28, H1 and L1.29, H1 and L1.30, H1 and L1.31, H1 and L1.32, H1 and L1.33, H1 and L1.34, H1 and L1.35, H1 and L1.36, H1 and L1.37, H1 and L1.38, H1 and L1.39, H1 and L1.40, H1 and L1.41, H1 and L1.42, H1 and L1.43, H1 and L1.44, H1 and L1.45, H1 and L1.46, H1 and L1.47, H1 and L1.48, H1 and L1.49, H1 and L1.50, H1 and L1.51, H1 and L1.52, H1 and L1.53, H1 and L1.54, H1 and L1.55, H1 and L1.56, H1 and L1.57, H1 and L1.58, H1 and L1.59, H1 and L1.60, H1 and L1.61, H1 and L1.62, H1 and L1.63, H1 and L1.64, H1 and L1.65, H1 and L1.66, H1 and L1.67, H1 and L1.68, H1 and L1.69, H1 and L1.70, H1 and L1.71, H1 and L1.72, H1 and L1.73, H1 and L1.74, H1 and L1.75, H1 and L1.76, H1 and L1.77, H1 and L1.78, H1 and L1.79, H1 and L1.80, H1 and L1.81, H1 and L1.82, H1 and L1.83, H1 and L1.84, H1 and L1.85, H1.42 and L1.47, H1.42 and L1.56, H1.190 and L1.3, H1.191 and L1.3, H1.192 and L1.3, H1.195 and L1.3, H1.196 and L1.3, H1.197 and L1.3, H1.198 and L1.3, H1.199 and L1.3, H1.200 and L1.3, H1.201 and L1.3, H1.202 and L1.3, H1.203 and L1.3, H1.204 and L1.3, H1.205 and L1.3, H1.206 and L1.3, H1.207 and L1.3, H1.208 and L1.3, H1.209 and L1.3, H1.210 and L1.3, H1.211 and L1.3, H1.212 and L1.3, H1.213 and L1.3, H1.214 and L1.3, H1.215 and L1.3, H1.216 and L1.3, H1.217 and L1.3, H1.218 and L1.3, H1.219 and L1.3, H1.220 and L1.3, H1.221 and L1.3, H1.222 and L1.3, H1.223 and L1.3, H1.224 and L1.3, H1.225 and L1.3, H1.226 and L1.3, H1.227 and L1.3, H1.228 and L1.3, H1.21 and L1.92, H1.21 and L1.93, H1.21 and L1.94, H1.21 and L1.95, H1.21 and L1.96, H1.21 and L1.97, H1.21 and L1.98, H1.21 and L1.99, H1.21 and L1.100, H1.21 and L1.101, H1.21 and L1.102, H1.21 and L1.103, H1.21 and L1.104, H1.21 and L1.105, H1.21 and L1.106, H1.21 and L1.107, H1.21 and L1.108, H1.21 and L1.109, H1.21 and L1.110, H1.21 and L1.111, H1.21 and L1.112, H1.21 and L1.113, H1.21 and L1.114, H1.21 and L1.115, H1.21 and L1.116, H1.21 and L1.117, H1.21 and L1.118, H1.21 and L1.119, H1.195 and L1.92, H1.195 and L1.94, H1.195 and L1.95, H1.195 and L1.104, H1.195 and L1.105, H1.195 and L1.109, H1.196 and L1.92, H1.196 and L1.94, H1.196 and L1.95, H1.196 and L1.104, H1.196 and L1.105, H1.196 and L1.109, H1.205 and L1.92, H1.205 and L1.94, H1.205 and L1.95, H1.205 and L1.104, H1.205 and L1.105, H1.205 and L1.109, H1.193 and L1.91, H1.194 and L1.86, H1.194 and L1.87, H1.194 and L1.88, H1.194 and L1.89, or H1.194 and L1.90, or H1.2320 and L1.100, or H1.230 and L1.115 (FIGS. 22, 31, 32 and 39). Additional VH and VL sequences of exemplary STEAP1 binding domains that can be used in the subject 2+1 Fab2-scFv-Fc format antibody are depicted in FIGS. 22, 31, and 32.

In some embodiments of the 2+1 Fab2-scFv-Fc format, the STEAP1 ABDs each include a VH and VL domain selected from the following: (i) a VH comprising a vhCDR1, a vhCDR2, and a vhCDR3 having an amino acid sequence of a vhCDR1, a vhCDR2, and a vhCDR3, respectively, of a VH of any of the STEAP1 binding domains depicted in FIG. 22 or a variant thereof; and (ii) a VL comprising a vlCDR1, a vlCDR2, and a vlCDR3 having an amino acid sequence of a vlCDR1, a vlCDR2, and a vlCDR3, respectively, of a VL of any of the STEAP1 binding domains depicted in FIG. 22 or a variant thereof; or (i) a VH comprising a vhCDR1, a vhCDR2, and a vhCDR3 having an amino acid sequence of a vhCDR1, a vhCDR2, and a vhCDR3, respectively, of a VH depicted in FIG. 22 or FIG. 31 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 22 or FIG. 32 or a variant thereof.

In some embodiments of the 2+1 Fab2-scFv-Fc format, the STEAP1 ABDs each include a VH and VL domain selected from the following: (i) a VH having an amino acid sequence of any of the VH of any of the STEAP1 binding domains depicted in FIG. 22 or a variant thereof; and (ii) a VL having an amino acid sequence of a VL of any of the STEAP1 binding domains depicted in FIG. 22 or a variant thereof; or (i) a VH having an amino acid sequence of a VH depicted in FIG. 22 or FIG. 31, or a variant thereof; and (ii) a VL having an amino acid sequence of a VL depicted in FIG. 22 or FIG. 32 or a variant thereof.

FIG. 9 shows some exemplary Fc domain sequences that are useful in the 2 +1 Fab2-scFv-Fc format antibodies. In addition, FIGS. 11 and 12 provide exemplary CH1-hinge domains, CH1 domains, and hinge domains that can be included in the first or second monomer of the 2+1 Fab2-scFv-Fc format. Further, FIG. 13 provides useful CL sequences that can be used with this format.

Exemplary anti-CD28 x anti-STEAP1 bispecific antibodies in the 2+1 Fab2-scFv-Fc format are depicted in FIG. 40. 4. 2+1 Fab2-Fc x scFv-Fc format

One heterodimeric antibody format that finds particular use in the subject anti-CD28 x anti-STEAP1 antibodies provided herein is the 2+1 Fab2-Fc x scFv-Fc format (also referred to as “stacked bottle opener”) shown in FIG. 14F. This format includes a first monomer, a second monomer and a common light chain. The first monomer includes, from N- to C-terminus: a VH1-CH1-domain linker-VH1-CH1-hinge-CH2-CH3, wherein the VH1s are each a first variable heavy domain and the CH2-CH3 is a first variant Fc domain. The domain linker can be any useful domain linker (see, e.g., FIG. 7). The second monomer includes a single-chain Fv (“scFv”) covalently attached to a second variant Fc domain by a domain linker (scFv-domain linker-CH2-CH3). The common light chain includes a VL1-CL wherein the VL1 is a first variable light domain. The scFv of the second monomer includes a second variable heavy domain (VH2) attached to a second variable light domain (VL2) by an scFv linker. In some embodiments, this format includes two identical common light chains (VL1-CL). In this embodiment, the two VH1-CH1s of the first monomer each interact with a common light chain to form two identical Fabs that are first antigen binding domains, and the VH2 and VL2 form a second antigen binding domain. In some embodiments, each of the first antigen binding domains are STEAP1 binding domains, and the second antigen binding domain is a CD28 binding domain. As for many of the embodiments herein, these constructs can include skew variants, pI variants, ablation variants, additional Fc variants, etc. as desired and described herein.

In some embodiments, the first and second Fc domains of the 2+1 Fab2-Fc x scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fe domains include ablation variants (including those shown in FIG. 5). In some embodiments, each of the first and second variant Fc domains include ablation variants E233P/L234V/L235A/G236del/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the 2+1 Fab2-Fc x scFv-Fc format antibody provided herein includes a charged scFv linker (including those shown in FIG. 6). In some embodiments, the 2+1 Fab2-scFv-Fc format antibody provided herein includes FcRn variants M428L/N434S, wherein numbering is according to EU numbering.

In exemplary embodiments, the 2+1 Fab2-Fc x scFv-Fc format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236del/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-Fc x scFv-Fc format antibody provided herein includes a (GKPGS)₄ charged scFv linker (SEQ ID NO: 24). In some embodiments, the 2+1 Fab2-Fc x scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-Fc x scFv-Fc format antibody is a CD28 binding domain and the VH1 of the first and second monomer and the VL1 of the common light chain each form binding domains that bind STEAP1 . Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: IA7[CD28]_H1L1, IA7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65).

In some embodiments of the 2+1 Fab2-Fc x scFv-Fc format, the anti-CD28 ABD has a VH and VL domain selected from the following: (i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or (i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIGS. 15, 17, or 82 or a variant thereof.

In some embodiments of the 2+1 Fab2-Fc x scFv-Fc, the anti-CD28 ABD has a VH and VL domain selected from the following:(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domains depicted in FIGS. 15, 18, 21 and 65 or a variant thereof; or (i) a VH having an amino acid sequence of a VH depicted in FIG. 15 or 16 or a variant thereof; and (ii) a VL having an amino acid sequence of a VL depicted in FIGS. 15, 17, or 82 or a variant thereof.

In some embodiments, the VH1 of the first and second monomer and the VL1 of the common light chain of the 2+1 Fab2-Fc x scFv-Fc format antibody each form a binding domain that binds STEAP1 . Any suitable STEAP1 binding domain can be included in subject 2+1 Fab2-Fc x scFv-Fc, including any of the STEAP1 binding domains provided herein. In some embodiments, the STEAP1 binding domain is one of the following STEAP1 binding domains or a variant thereof. H1 and L1, H1.1 and L1, H1.2 and L1, H1.3 and L1, H1.4 and L1, H1.5 and L1, H1.6 and L1, H1.7 and L1, H1.8 and L1, H1.9 and L1, H1.10 and L1, H1.11 and L1, H1.12 and L1, H1.13 and L1, H1.14 and L1, H1.15 and L1, H1.16 and L1, H1.17 and L1, H1.18 and L1, H1.19 and L1, H1.20 and L1, H1.21 and L1, H1.22 and L1, H1.23 and L1, H1.24 and L1, H1.25 and L1, H1.26 and L1, H1.27 and L1, H1.28 and L1, H1.29 and L1, H1.30 and L1, H1.31 and L1, H1.32 and L1, H1.33 and L1, H1.34 and L1, H1.35 and L1, H1.36 and L1, H1.37 and L1, H1.38 and L1, H1 and L1.1, H1 and L1.2, H1 and L1.3, H0 and L0, H1.21 and L1.3, H1.34 and L1.3, H1.39 and L1, H1.40 and L1, H1.4I and L1, H1.42 and L1, H1.43 and L1, H1.44 and L1, H1.45 and L1, H1.46 and L1, H1.47 and L1, H1.48 and L1, H1.49 and L1, H1.50 and L1, H1.51 and L1, H1.52 and L1, H1.53 and L1, H1.54 and L1, H1.55 and L1, H1.56 and L1, H1.57 and L1, H1.58 and L1, H1.59 and L1, H1.60 and L1, H1.61 and L1, H1.62 and L1, H1.63 and L1, H1.64 and L1, H1.65 and L1, H1.66 and L1, H1.67 and L1, H1.68 and L1, H1.69 and L1, H1.70 and L1, H1.71 and L1, H1.72 and L1, H1.73 and L1, H1.74 and L1, H1.75 and L1, H1.76 and L1, H1.77 and L1, H1.78 and L1, H1.79 and L1, H1.80 and L1, H1.81 and L1, H1.82 and L1, H1.83 and L1, H1.84 and L1, H1.85 and L1, H1.86 and L1, H1.87 and L1, H1.88, and L1, H1.89 and L1, H1.90 and L1, H1.91 and L1, H1.92 and L1, H1.93 and L1, H1.94 and L1, H1.95 and L1, H1.96 and L1, H1.97 and L1, H1.98 and L1, H1.99 and L1, H1.100 and L1, H1.101 and L1, H1.102 and L1, H1.103 and L1, H1.104 and L1, H1.105 and L1, H1.106 and L1, H1.107 and L1, H1.108 and L1, H1.109 and L1, H1.110 and L1, H1.III and L1, H1.112 and L1, H1.113 and L1, H1.114 and L1, H1.115 and L1, H1.116 and L1, H1.117 and L1, H1.118 and L1, H1.119 and L1, H1.120 and L1, H1.121 and L1, H1.122 and L1, H1.123 and L1, H1.124 and L1, H1.125 and L1, H1.126 and L1, H1.127 and L1, H1.128 and L1, H1.129 and L1, H1.130 and L1, H1.131 and L1, H1.132 and L1, H1.133 and L1, H1.134 and L1, H1.135 and L1, H1.136 and L1, H1.137 and L1, H1.138 and L1, H1.139 and L1, H1.140 and L1, H1.141 and L1, H1.142 and L1, H1.143 and L1, H1.144 and L1, H1.145 and L1, H1.146 and L1, H1.147 and L1, H1.148 and L1, H1.149 and L1, H1.150 and L1, H1.151 and L1, H1.152 and L1, H1.153 and L1, H1.154 and L1, H1.155 and L1, H1.156 and L1, H1.157 and L1, H1.158 and L1, H1.159 and L1, H1.160 and L1, H1.161 and L1, H1.162 and L1, H1.163 and L1, H1.164 and L1, H1.165 and L1, H1.166 and L1, H1.167 and L1, H1.168 and L1, H1.169 and L1, H1.170 and L1, H1.171 and L1, H1.172 and L1, H1.173 and L1, H1.174 and L1, H1.175 and L1, H1.176 and L1, H1.177 and L1, H1.178 and L1, H1.179 and L1, H1.180 and L1, H1.181 and L1, H1.182 and L1, H1.183 and L1, H1.184 and L1, H1.185 and L1, H1.186 and L1, H1.187 and L1, H1.188 and L1, H1.189 and L1, H1 and L1.4, H1 and L1.5, H1 and L1.6, H1 and L1.7, H1 and L1.8, H1 and L1.9, H1 and L1.10, H1 and L1.11, H1 and L1.12, H1 and L1.13, H1 and L1.14, H1 and L1.15, H1 and L1.16, H1 and L1.17, H1 and L1.18, H1 and L1.19, H1 and L1.20, H1 and L1.21, H1 and L1.22, H1 and L1.23, H1 and L1.24, H1 and L1.25, H1 and L1.26, H1 and L1.27, H1 and L1.28, H1 and L1.29, H1 and L1.30, H1 and L1.31, H1 and L1.32, H1 and L1.33, H1 and L1.34, H1 and L1.35, H1 and L1.36, H1 and L1.37, H1 and L1.38, H1 and L1.39, H1 and L1.40, H1 and L1.41, H1 and L1.42, H1 and L1.43, H1 and L1.44, H1 and L1.45, H1 and L1.46, H1 and L1.47, H1 and L1.48, H1 and L1.49, H1 and L1.50, H1 and L1.51, H1 and L1.52, H1 and L1.53, H1 and L1.54, H1 and L1.55, H1 and L1.56, H1 and L1.57, H1 and L1.58, H1 and L1.59, H1 and L1.60, H1 and L1.61, H1 and L1.62, H1 and L1.63, H1 and L1.64, H1 and L1.65, H1 and L1.66, H1 and L1.67, H1 and L1.68, H1 and L1.69, H1 and L1.70, H1 and L1.71, H1 and L1.72, H1 and L1.73, H1 and L1.74, H1 and L1.75, H1 and L1.76, H1 and L1.77, H1 and L1.78, H1 and L1.79, H1 and L1.80, H1 and L1.81, H1 and L1.82, H1 and L1.83, H1 and L1.84, H1 and L1.85, H1.42 and L1.47, H1.42 and L1.56, H1.190 and L1.3, H1.191 and L1.3, H1.192 and L1.3, H1.195 and L1.3, H1.196 and L1.3, H1.197 and L1.3, H1.198 and L1.3, H1.199 and L1.3, H1.200 and L1.3, H1.201 and L1.3, H1.202 and L1.3, H1.203 and L1.3, H1.204 and L1.3, H1.205 and L1.3, H1.206 and L1.3, H1.207 and L1.3, H1.208 and L1.3, H1.209 and L1.3, H1.210 and L1.3, H1.211 and L1.3, H1.212 and L1.3, H1.213 and L1.3, H1.214 and L1.3, H1.215 and L1.3, H1.216 and L1.3, H1.217 and L1.3, H1.218 and L1.3, H1.219 and L1.3, H1.220 and L1.3, H1.221 and L1.3, H1.222 and L1.3, H1.223 and L1.3, H1.224 and L1.3, H1.225 and L1.3, H1.226 and L1.3, H1.227 and L1.3, H1.228 and L1.3, H1.21 and L1.92, H1.21 and L1.93, H1.21 and L1.94, H1.21 and L1.95, H1.21 and L1.96, H1.21 and L1.97, H1.21 and L1.98, H1.21 and L1.99, H1.21 and L1.100, H1.21 and L1.101, H1.21 and L1.102, H1.21 and L1.103, H1.21 and L1.104, H1.21 and L1.105, H1.21 and L1.106, H1.21 and L1.107, H1.21 and L1.108, H1.21 and L1.109, H1.21 and L1.110, H1.21 and L1.111, H1.21 and L1.112, H1.21 and L1.113, H1.21 and L1.114, H1.21 and L1.115, H1.21 and L1.116, H1.21 and L1.117, H1.21 and L1.118, H1.21 and L1.119, H1.195 and L1.92, H1.195 and L1.94, H1.195 and L1.95, H1.195 and L1.104, H1.195 and L1.105, H1.195 and L1.109, H1.196 and L1.92, H1.196 and L1.94, H1.196 and L1.95, H1.196 and L1.104, H1.196 and L1.105, H1.196 and L1.109, H1.205 and L1.92, H1.205 and L1.94, H1.205 and L1.95, H1.205 and L1.104, H1.205 and L1.105, H1.205 and L1.109, H1.193 and L1.91, H1.194 and L1.86, H1.194 and L1.87, H1.194 and L1.88, H1.194 and L1.89, or H1.194 and L1.90, or H1.2320 and L1.100, or H1.230 and L1.115 (FIGS. 22, 31, 32 and 39). Additional VH and VL sequences of exemplary STEAP1 binding domains that can be used in the subject 2+1 Fab2-Fc x scFv-Fc format antibody are depicted in FIGS. 22, 31, and 32.

In some embodiments of the 2+1 Fab2-Fc x scFv-Fc format, the STEAP1 ABDs each include a VH and VL domain selected from the following: (i) a VH comprising a vhCDR1, a vhCDR2, and a vhCDR3 having an amino acid sequence of a vhCDR1, a vhCDR2, and a vhCDR3, respectively, of a VH of any of the STEAP1 binding domains depicted in FIG. 22 or a variant thereof; and (ii) a VL comprising a vlCDR1, a vlCDR2, and a vlCDR3 having an amino acid sequence of a vlCDR1, a vlCDR2, and a vlCDR3, respectively, of a VL of any of the STEAP1 binding domains depicted in FIG. 22 or a variant thereof; or (i) a VH comprising a vhCDR1, a vhCDR2, and a vhCDR3 having an amino acid sequence of a vhCDR1, a vhCDR2, and a vhCDR3, respectively, of a VH depicted in FIG. 22 or FIG. 31 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 22 or FIG. 32 or a variant thereof.

In some embodiments of the 2+1 Fab2-Fc x scFv-Fc, the STEAP1 ABDs each include a VH and VL domain selected from the following: (i) a VH having an amino acid sequence of any of the VH of any of the STEAP1 binding domains depicted in FIG. 22 or a variant thereof; and (ii) a VL having an amino acid sequence of a VL of any of the STEAP1 binding domains depicted in FIG. 22 or a variant thereof; or (i) a VH having an amino acid sequence of a VH depicted in FIG. 22 or FIG. 31 or a variant thereof; and (ii) a VL having an amino acid sequence of a VL depicted in FIG. 22 or FIG. 32 or a variant thereof.

FIG. 9 shows some exemplary Fc domain sequences that are useful in 2+1 Fab2-Fc x scFv-Fc format antibodies. In addition, FIGS. 11 and 12 provide exemplary CH1-hinge domains, CH1 domains, and hinge domains that can be included in the first or second monomer of the 2+1 Fab2-Fc x scFv-Fc format. Further, FIG. 13 provides useful CL sequences that can be used with this format.

### 5. 1+1 CLC Format

One heterodimeric antibody format that finds particular use in subject anti-CD28 x anti-STEAP1 antibodies provided herein is the “1+1 Common Light Chain” or “1+1 CLC” format, which is depicted in FIG. 14C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CHT-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen binding specificity; and the VL pairs with the VH2 to form a second binding domain with a second antigen binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236del/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236del/S267K; and the constant domain (CH1-hinge-CH2-CH3) of the first monomer includes pI variants N208D/Q295E/N384D/Q418E/N421D, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the 1+1 CLC format antibody provided herein further includes FcRn variants M428L/N434S, wherein numbering is according to EU numbering.

In some embodiments, one of the first binding domain or the second binding domain binds CD28 and the other binding domain binds STEAP1 . Any suitable CD28 binding domain and STEAP1 domain can be included in subject 1+1 CLC format antibody, including any of the CD28 binding domains and STEAP1 binding domains provided herein or a variant thereof (see, e.g., FIGS. 15-18, 21, 22, 31, 32, and 82).

### 6. 2+1 CLC Format

Another heterodimeric antibody format that finds particular use in subject CD28 x anti-STEAP1 antibodies provided herein is the “2+1 Common Light Chain” or “2+1 CLC” format, which is depicted in FIG. 14D. The 2+1 CLC format includes a first monomer that includes a VH1-CH1-linker-VH1-CH1-hinge-CH2-CH3, wherein the VHls are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VHls of the first monomer to form two first binding domains, each with a first antigen binding specificity; and the VL pairs with the VH2 to form a second binding domain with a second antigen binding specificity. The linker of the first monomer can be any suitable linker, including any one of the domain linkers or combinations thereof described in FIG. 7. In some embodiments, the 2+1 CLC format antibody is a trivalent antibody.

In some embodiments, the first and second Fe domains of the 2+1 CLC format are variant Fe domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236del/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236del/S267K; and the constant domain (CHT-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267 K, and the first Fc domain comprises amino acid variants S364K/E357Q/E233P/L234V/L235A/G236del/S267K, wherein numbering is according to EU numbering.

In some embodiments, each of the two first binding domains binds a tumor STEAP1 and the second binding domain binds CD28. Any suitable CD28 binding domain and STEAP1 domain can be included in the subject 2+1 CLC format antibody, including any of the CD28 binding domains and STEAP1 binding domains provided herein or a variant thereof (see, e.g., FIGS. 15-18, 21, 22, 31, 32, and 82). 7. Dual scFv formats

One heterodimeric antibody format that finds particular use in the subject bispecific anti-CD28 x anti-STEAP1 antibodies is the dual scFv format, as are known in the art and shown in FIG. 14G. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation.

In this case, all ABDs are in the scFv format. Any suitable STEAP1 binding domain and CD28 binding domain can be included in anti-CD28 x anti-STEAP1 antibody in the dual scFv format, including any of the STEAP1 binding domains and CD28 binding domains provided herein.

In addition, the Fc domains of the dual scFv format comprise skew variants (e.g. a set of amino acid substitutions as shown in FIGS. 3 and 8, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 5), optionally charged scFv linkers (including those shown in FIG. 6) and the heavy chain comprises pI variants (including those shown in FIG. 4).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable STEAP1 binding domain and CD28 binding domain can be included in the dual scFv format, including any of the STEAP1 binding domains and CD28 binding domains and related VHs and VLs provided herein or a variant thereof (see, e.g., FIGS. 15-18, 21, 22, 31, 32, and 82). 8. One-armed central-scFv

One heterodimeric antibody format that finds particular use in subject anti-CD28 x anti-STEAP1 antibodies provided herein is the one-armed central-scFv format shown in FIG. 14K. In this embodiment, one monomer comprises just an Fc domain, while the other monomer includes a Fab domain (a first antigen binding domain), a scFv domain (a second antigen binding domain) and an Fc domain, where the scFv domain is inserted between the Fab domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable STEAP1 binding domain and CD28 binding domain can be included in the one-armed central-scFv format, including any of the STEAP1 binding domains and CD28 binding domains and related VHs and VLs provided herein or a variant thereof (see, e.g., FIGS. 15-18, 21, 22, 31, 32, and 82). 9. One-armed scFv-mAb format

One heterodimeric antibody format that finds particular use in subject anti-CD28 x anti-STEAP1 antibodies provided herein is the one-armed sc-Fv-mAb format shown in FIG. 14H. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds STEAP1.

As for many of the embodiments herein, these constructs include skew variants, pI variants, ablation variants, additional Fc variants, etc. as desired and described herein.

Any suitable STEAP1 binding domain and CD28 binding domain can be included in the one-armed scFv-mAb format antibody, including any of the STEAP1 binding domains and CD28 binding domains and related VHs and VLs provided herein or a variant thereof (see, e.g., FIGS. 15-18, 21, 22, 31, 32, and 82). 10. scFv-mAb format

One heterodimeric antibody format that finds particular use in subject anti-CD28 x anti-STEAP1 antibodies provided herein is the mAb-scFv format shown in FIG. 141. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vhl-scFv linker-vll-[optional domain linker]- vh2-CH1-hinge-CH2-CH3) or (with the scFv in the opposite orientation) ((vll-scFv linker-vhl-[optional domain linker]-vh2-CHT-hinge-CH2-CH3)). The second monomer comprises a heavy chain VH2-CH1-hinge-CH2-CH3. This embodiment further utilizes a common light chain, comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable STEAP1 binding domain and CD28 binding domain can be included in the scFv-mAb antibody format, including any of the STEAP1 binding domains and CD28 binding domains and related VHs and VLs provided herein or a variant thereof (see, e.g., FIGS. 15-18, 21, 22, 31, 32, and 82). 11. mAb-Fv format

One heterodimeric antibody format that finds particular use in subject anti-CD28 x anti-STEAP1 antibodies provided herein is the mAb-Fv format (FIG. 14L). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind CD28 and the “extra” Fv domain binds STEAP1.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vhl-CHT-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vhl-CHT-hinge-CH2-CH3-[optional linker]-vh2). This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

Any suitable STEAP1 binding domain and CD28 binding domain can be included in the mAb-Fv format, including any of the STEAP1 binding domains and CD28 binding domains and related VHs and VLs provided herein or a variant thereof (see, e.g., FIGS. 15-18, 21, 22, 31, 32, and 82). 12. Central-Fv format

One heterodimeric antibody format that finds particular use in subject anti-CD28 x anti-STEAP1 antibodies provided herein is the central-Fv format shown in FIG. 14M. In this embodiment, the format relies on the use of an inserted Fv domain thus forming an “extra” third antigen binding domain, wherein the Fab portions of the two monomers bind STEAP1 and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vhl-CH1-[optional linker]-vl2-hinge-CH2-CH3). The other monomer comprises a first heavy chain comprising a first variable heavy domain, a CH1 domain and Fc domain and an additional variable heavy domain (vhl-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain, comprising a variable light domain and a constant light domain, that associates with the heavy chains to form two identical Fabs that each bind STEAP1 . The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable STEAP1 binding domain and CD28 binding domain can be included in the central-Fv format, including any of the STEAP1 binding domains and CD28 binding domains and related VHs and VLs provided herein or a variant thereof (see, e.g., FIGS. 15-18, 21, 22, 31, 32, and 82). 13. Non-heterodimeric bispecific antibodies

As will be appreciated by those in the art, the anti-CD28 x anti-STEAP1 antibodies provided herein can also be included in non-heterodimeric bispecific formats (see FIG. 14J). In this format, the anti-CD28 x anti-STEAP1 includes: 1) a first monomer comprising a VH1-CHT-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds STEAP1.

Any suitable STEAP1 binding domain and CD28 binding domain can be included in anti-CD28 x anti-STEAP1 antibody in the non-heterodimeric bispecific antibody format, including any of the STEAP1 binding domains and CD28 binding domains and related VHs and VLs provided herein or a variant thereof (see, e.g., FIGS. 15-18, 21, 22, 31, 32, and 82). 14. Trident Format

In some embodiments, the anti-CD28 x anti-STEAP1 antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 14N. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 14N, the second and third ABDs bind the same antigen.

Any suitable STEAP1 binding domain and CD28 binding domain can be included in the trident format, including any of the STEAP1 binding domains and CD28 binding domains and related VHs and VLs provided herein or a variant thereof (see, e.g., FIGS. 15-18, 21, 22, 31, 32, and 82).

V. Anti-CD28 x Anti-CEACAM5 Antibodies

In one aspect, provided herein are novel anti-CD28 x anti-CEACAM5 antibodies In some embodiments, the anti-CD28 x anti-CEACAM5 antibodies described herein are capable of agonistically binding to CD28 costimulatory molecules on T cells and CEACAM5 on tumor cells. Such antibodies selectively enhance anti-tumor activity at CEACAM5-associated tumor sites while minimizing peripheral toxicity. The subject antibodies provided herein are particularly useful in combination with other anti-cancer therapies, including, for example, bispecific antibodies for the treatment of CEACAM5-associated cancers.

The anti-CD28 x anti-CEACAM5 antibodies are multivalent and include at least two antigen binding domains (ABDs), wherein at least one antigen binding domain is a CD28 binding domain and at least one antigen binding domain is a CEACAM5 binding domain. Any suitable CD28 binding domain and CEACAM5 binding domain can be included in the subject anti-CD28 x anti-CEACAM5 antibodies, including, for example, the CD28 binding domains and CEACAM5 binding domains provided herein.

The antigen binding domains provided herein generally include a variable heavy domain (VH) having a VH-CDR1, VH-CDR-2, and VH-CDR-3; and a variable light domain (VL), and a variable light domain (VL) having a VL-CDR1, VL-CDR-2, and VL-CDR-3.

In addition, as discussed above, the numbering used in the sequence listing and figures for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85, 90, 95 or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85, 95 to 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain).

In addition, in embodiments wherein the subject antibody includes an scFv, the scFv can be in an orientation from N- to C-terminus ofVH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIG. 6.

In some embodiments, the anti-CD28 x anti-CEACAM5 antibody is a bispecific antibody. In some embodiments, the anti-CD28 x anti-CEACAM5 antibody is a bivalent antibody. In some embodiments, the anti-CD28 x anti-CEACAM5 antibody is a trivalent antibody. In some embodiments, the anti-CD28 x anti-CEACAM5 antibody is a bispecific, bivalent antibody. In some embodiments, the anti-CD28 x anti-CEACAM5 antibodies include one CD28 binding domain and one CEACAM5 binding domain. In exemplary embodiments, the anti-CD28 x anti-CEACAM5 antibody is a bispecific, trivalent antibody. In some embodiments, the anti-CD28 x anti-CEACAM5 antibodies include one CD28 binding domain and two CEACAM5 binding domains.

The anti-CD28 x anti-CEACAM5 antibodies provided herein can be in any useful format, including, including, for example, canonical immunoglobulin, as well as the “1 +1 Fab-scFv-Fc,” “2+1 “mAb-scFv,” “2+1 Fab2-scFv-Fc,” and “2+1 Fab2-Fc x scFv-Fc” formats described herein (FIG. 14). Additional useful formats include, but are not limited to: “mAb-Fv,” “central-Fv,” “one-armed scFv-mAb,” “scFv-mAb,” “dual scFv,” and “trident” formats provided herein (see, e.g., FIG. 14). See also, US20180127501A1, which is incorporated by reference herein, particularly in pertinent part relating to antibody formats (see, e.g., FIG. 2). In some embodiments, the anti-CD28 x anti-CEACAM5 antibodies are heterodimeric bispecific antibodies that include variant Fc domains having any of the heterodimerization skew variants, pI variants and/or ablation variants described herein. See, e.g. FIG. 8.

Note that unless specified herein, the order of the antigen list in the name does not confer structure; that is an anti-CEACAM5 x anti-CD28 1+1 Fab-scFv-Fc antibody can have the scFv bind to CEACAM5 or CD28, although in some cases, the order specifies structure as indicated.

The anti-CD28 x anti-CEACAM5 antibodies provided herein further include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CHT-hinge-Fc domain or CHT-hinge-CH2-CH3), the variable heavy domain, the variable light domain, the light constant domain, Fab domains and scFv domains.

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr.

The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 2684), (GGGGS)n (SEQ ID NO: 36), and (GGGS)n (SEQ ID NO: 2685), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example, the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker,” used to link any two domains as outlined herein together. For example, in the 2+1 Fab2-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 2684), (GGGGS)n (SEQ ID NO: 36), and (GGGS)n (SEQ ID NO: 2685), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness,” as outlined below, charged domain linkers, as used in some embodiments of scFv linkers can be used. Exemplary useful domain linkers are depicted in FIG. 7.

In some embodiments, the linker is a scFv linker that is used to covalently attach the VH and VL domains as discussed herein. In many cases, the scFv linker is a charged scFv linker, a number of which are shown in FIG. 6. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers.

Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the invention as well, and thus those included in FIG. 7 can be used in any embodiment herein where a linker is utilized.

Exemplary subject anti-CD28 x anti-CEACAM5 antibodies are depicted, for example, in FIG. 24. During the cell culture production of the anti-CD28 x anti-CEACAM5 antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-CD28 x anti-CEACAM5 antibody is a variant of one of the anti-CD28 x anti-CEACAM5 antibodies includes a deletion of a C-terminal lysine (-K) terminal or C-terminal lysine and glycine (-GK) residues in one or both Fe domains of an anti-CD28 x anti-CEACAM5 antibody described herein. In some embodiments, the deletion is G446del and/or K447del (EU numbering).

Aspects of the anti-CD28 x anti-CEACAM5 antibodies are further described in detail below.

### A. CD28 Binding Domains

In one aspect, provided herein are CD28 antigen binding domain (ABD) compositions. In embodiments, the CD28 ABDs include set of 6 CDRs as depicted in the sequence listing and FIG. 65 either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIG. 65 and the sequence listing (see Table 2). Suitable CD28 ABDs can also include the entire VH and VL sequences as depicted in these sequences and figures, used as scFvs or as Fab domains. In embodiments, the CD28 ABD includes a variable heavy (VH) domain or variant thereof and/or variable light (VL) domain or variant thereof of any of the CD28 binding domains disclosed herein (e.g., FIG. 65). Such CD28 binding domains are useful, for example, for inclusion in the anti-CD28 x anti- CEACAM5 antibodies provided herein.

In exemplary embodiments, the CD28 ABD includes the variable heavy (VH) domain and variable light (VL) domain of any one of the CD28 ABDs described herein, including the figures and sequence listing. In exemplary embodiments, the CD28 ABD is a CD28 ABD depicted in FIG. 65. In exemplary embodiments, the CD28 ABD comprises a VH and VL pair selected from those depicted in FIG. 65.

In addition to the parental CD28 variable heavy and variable light domains disclosed herein, provided herein are CD28 ABDs that include a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a CD28 ABD depicted in FIG. 65. In some embodiments, the changes are in a VH domain depicted in FIG. 65. In some embodiments, the changes are in a VL domain are depicted in FIG. 65. In some embodiments, the changes are in a VH and VL domain are depicted in FIG. 65. In some embodiments, the one or more amino acid changes are in the VH and/or VL framework regions (FR1, FR2, FR3, and/or FR4). In some embodiments, the one or more amino acid change(s) are in one or more of vhCDR1-3 and/or vlCDR1-3. In some embodiments, the CD28 variant includes the vhCDR1-3 and/or vlCDR1-3 of any of the CD28 ABDs in FIG. 65. In certain embodiments, the CD28 ABD is capable of binding to CD28, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (FIG. 2).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of a CD28 ABD depicted in FIG. 65. In some embodiments, the CD28 ABD includes a VH that is at least 90, 95, 97, 98 or 99% identical to VH domain depicted in FIG. 65. In some embodiments, the CD28 ABD includes a VL that is at least 90, 95, 97, 98 or 99% identical to VL domain depicted in FIG. 65. In some embodiments, the CD28 ABD includes a VH and a VL that is at least 90, 95, 97, 98 or 99% identical to a VH domain and a VL domain depicted in FIG. 65. In some embodiments, the CD28 ABD includes the 6 CDRs (vhCDR1-3 and vlCDR1-3) of any of the CD28 ABDs of FIG. 65. In certain embodiments, the CD28 is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (FIG. 2).

The anti-CD28 x anti-CEACAM5 antibodies provided herein include at least one CD28 binding domain. Any suitable CD28 binding domain can be included in the anti-CD28 x anti-CEACAM5 antibodies provided herein. In exemplary embodiments, the CD28 binding domain is an agonistic CD28 ABDs that advantageously provide T cell costimulatory activity.

As will be appreciated by those in the art, suitable CD28 binding domains can comprise a set of 6 CDRs as depicted in the figures, either as they are underlined or, in the case where a different numbering scheme is used, as described herein and as shown in Table 2, as the CDRs that are identified using other alignments within the variable heavy (VH) domain and variable light domain (VL) sequences of those CD28 binding domains depicted in FIGS. 15, 18, 21 and 65. Additional VH and VL sequences of exemplary CD28 binding domains that can be used in the subject antibodies are depicted in FIGS. 16 and 17. Suitable CD28 ABDs can also include the entire VH and VL sequences as depicted in these sequences and figures, used as scFvs or as Fabs.

In one embodiment, the CD28 antigen binding domain includes the 6 CDRs (i.e., vhCDR1-3 and vlCDR1-3) of any of the CD28 binding domains described herein, including, but not limited to those depicted in FIGS. 15, 18, 21 and 65. In some embodiments, the CD28 ABD that binds human CD28 is one of the following CD28 ABDs: 1A7[CD28]_H1L1, 1A7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the CD28 ABD includes a VH/VL pair selected from the VHs and VLs depicted in FIGS. 15, 16, 17, and 82.

In addition to the parental CDR sets disclosed in the figures and sequence listing that form an ABD to CD28, provided herein are variant CD28 ABDS having CDRs that include at least one modification of the CD28 ABD CDRs disclosed herein (e.g., (FIGS. 15, 18, 21 and 65, and the sequence listing). In one embodiment, the CD28 ABD of the subject anti-CD28 x anti-CEACAM5 antibody includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD of the subject anti-CD28 x anti-CEACAM5 antibody includes a set of 6 CDRs with 1, 2, 3, 4, 5, 6, 7, 8, 9, 10 amino acid modifications as compared to the 6 CDRs of one of the following CD28 ABDs: IA7[CD28]_H1L1, IA7[CD28]_H1.1_L1, IA7[CD28]_H1_L1.71, IA7[CD28]_H1.1_L1.71, 1A7[CD28]H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In certain embodiments, the CD28 ABD of the subject anti-CD28 x anti-CEACAM5 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 ABD of the subject anti-CD28 x anti-CEACAM5 antibody includes 6 CDRs that are at least 90, 95, 97, 98 or 99% identical to the 6 CDRs of a CD28 ABD as described herein, including the figures and sequence listing. In exemplary embodiments, the CD28 ABD of the subject anti-CD28 x anti-CEACAM5 antibody includes 6 CDRs that are at least 90, 95, 97, 98 or 99% identical to the 6 CDRs of one of the following CD28 ABDs: IA7[CD28]H1L1, IA7[CD28]_H1.1_L1, IA7[CD28]_H1_L1.71, IA7[CD28]_H1.1_L1.71, IA7[CD28]_H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], 1A7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65). In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In another exemplary embodiment, the CD28 ABD of the subject anti-CD28 x anti-CEACAM5 antibody includes the variable heavy (VH) domain and variable light (VL) domain of any one of the CD28 ABDs described herein, including the figures and sequence listing. In exemplary embodiments, the CD28 ABD is one of the following CD28 ABDs: IA7[CD28]_H1L1, IA7[CD28]_H1.1_L1, IA7[CD28]_H1_L1.71, IA7[CD28]_H1.1_L1.71, IA7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the CD28 ABD includes a VH and VL pair selected from the VH and VLs depicted in FIGS. 15, 16, 17, and 82.

In some embodiments, the anti-CD28 x anti-CEACAM5 antibody includes a CD28 ABD that includes a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD28 ABDs: IA7[CD28]_H1L1, IA7[CD28]_H1.1_L1, IA7[CD28]_H1_L1.71, IA7[CD28]_H1.1_L1.71, IA7[CD28]_H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1,TN228[CD28]_H4L2, IA7[CD28]H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the changes are in a VH domain depicted in FIGS. 15-18, 21 and 65. In some embodiments, the changes are in a VL domain are depicted in FIGS. 15-18, 21 and 65. In some embodiments, the changes are in a VH and VL domain are depicted in FIGS. 15-18, 21 and 65. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of one of the following CD28 ABDs: 1A7[CD28]_H1L1, 1A7[CD28]H1.1_L1, 1A7[CD28]_H1_L1.71, 1A7[CD28]_H1.1_L1.71, 1A7[CD28]H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). In some embodiments, the CD28 ABD includes a VH that is at least 90, 95, 97, 98 or 99% identical to VH domain depicted in FIGS. 15-18, 21 and 65. In some embodiments, the CD28 ABD includes a VL that is at least 90, 95, 97, 98 or 99% identical to VL domain depicted in FIGS. 15-18, 21, 65 and 82.

In some embodiments, the CD28 ABD includes a VH and a VL that is at least 90, 95, 97, 98 or 99% identical to a VH domain and a VL domain depicted in FIGS. 15-18, 21 and 65. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 binding domain of the subject anti-CD28 x anti-CEACAM5 antibody includes a VH that includes any one of the VHCDRI-3 or HFR1-4 sequences depicted in FIG. 19A. In some embodiments, the CD28 binding domain includes a VL that includes any one of the VLCDR1-3 or LFR1-4 sequences depicted in FIG. 19B.

In some embodiments, the anti-CD28 x anti-CEACAM5 antibody includes a CD28 binding domain that includes a VH and VL selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 15, 17, or 82 or a variant thereof.

In some embodiments, the anti-CD28 x anti-CEACAM5 antibody includes a CD28 binding domain that includes a VH and VL selected from the following:
(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or
(i) a VH having an amino acid sequence of a VH depicted in FIG. 15 or 16 or a variant thereof; and (ii) a VL having an amino acid sequence of the VL of a VL depicted in FIG. 15, 17, or 82 or a variant thereof.

In another aspect, provided herein is a CD28 binding domain that competes with any of the CD28 binding domains disclosed herein for binding to human CD28.

### B. CEACAM5 Binding Domains

In one aspect, provided herein are CEACAM5 antigen binding domain (ABD) compositions. In embodiments, the CEACAM5 ABDs include set of 6 CDRs as depicted in the sequence listing and FIG. 45 either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIG. 45 and the sequence listing (see Table 2). Suitable CEACAM5 ABDs can also include the entire VH and VL sequences as depicted in these sequences and figures, used as scFvs or as Fab domains. In embodiments, the CEACAM5 ABD includes a variable heavy (VH) domain or variant thereof and/or variable light (VL) domain or variant thereof of any of the CEACAM5 binding domains disclosed herein (e.g., FIG. 45). Such CEACAM5 binding domains are useful, for example, for inclusion in the anti-CD28 x anti- CEACAM5 antibodies provided herein.

In exemplary embodiments, the CEACAM5 ABD includes the variable heavy (VH) domain and variable light (VL) domain of any one of the CEACAM5 ABDs described herein, including the figures and sequence listing. In exemplary embodiments, the CEACAM5 ABD is a CEACAM5 ABD depicted in FIG. 45. In exemplary embodiments, the CEACAM5 ABD comprises a VH and VL pair selected from those depicted in FIG. 45.

In addition to the parental CEACAM5 variable heavy and variable light domains disclosed herein, provided herein are CEACAM5 ABDs that include a variable heavy domain and/or a variable light domain that are variants of a CEACAM5 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CEACAM5 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a CEACAM5 ABD depicted in FIG. 45. In some embodiments, the changes are in a VH domain depicted in FIG. 45. In some embodiments, the changes are in a VL domain are depicted in FIG. 45. In some embodiments, the changes are in a VH and VL domain are depicted in FIG. 45. In some embodiments, the one or more amino acid changes are in the VH and/or VL framework regions (FR1, FR2, FR3, and/or FR4). In some embodiments, the one or more amino acid change(s) are in one or more of vhCDR1-3 and/or vlCDR1-3. In some embodiments, the CEACAM5 variant includes the vhCDR1-3 and/or vlCDR1-3 of any of the CEACAM5 ABDs in FIG. 45. In certain embodiments, the CEACAM5 ABD is capable of binding to CEACAM5, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CEACAM5 ABD is capable of binding human CEACAM5 antigen (FIG. 2).

In one embodiment, the variant VH and/or VL domain is at least 90, 95, 97, 98 or 99% identical to the VH and/or VL of a CEACAM5 ABD as described herein, including the figures and sequence listing. In exemplary embodiments, the variant VH and/or VL domain is at least 90, 95, 97, 98 or 99% identical to the VH and/or VL of a CEACAM5 ABD depicted in FIG. 45. In some embodiments, the CEACAM5 ABD includes a VH that is at least 90, 95, 97, 98 or 99% identical to VH domain depicted in FIG. 45. In some embodiments, the CEACAM5 ABD includes a VL that is at least 90, 95, 97, 98 or 99% identical to VL domain depicted in FIG. 45. In some embodiments, the CEACAM5 ABD includes a VH and a VL that is at least 90, 95, 97, 98 or 99% identical to a VH domain and a VL domain depicted in FIG. 45. In some embodiments, the CEACAM5 ABD includes the 6 CDRs (vhCDR1-3 and vlCDR1-3) of any of the CEACAM5 ABDs of FIG. 45. In certain embodiments, the CEACAM5 is capable of binding to the CEACAM5, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CEACAM5 ABD is capable of binding human CEACAM5 antigen (FIG. 2).

The anti-CD28 x anti-CEACAM5 antibodies provided herein include at least one CEACAM5 binding domain. Subject antibodies that include such CEACAM5 antigen binding domains (e.g., anti-CEACAM5 x anti-CD3 bispecific antibodies) advantageously target cells that express high levels of CEACAM5 over those that express levels of CEACAM5 (e.g., normal cells).

In some embodiments, the CEACAM5 ABD of the anti-CD28 x anti-CEACAM5 antibody includes the set of 6 CDRs as depicted in the sequence listing and FIG. 41 and FIG. 45, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIG. 41 and FIG. 45 and the sequence listing (see Table 2).

In exemplary embodiments, the CEACAM5 ABD of the anti-CD28 x anti-CEACAM5 antibody includes the variable heavy (VH) domain and variable light (VL) domain of any one of the CEACAM5 ABDs described herein, including the figures and sequence listing. In exemplary embodiments, the CEACAM5 ABD of the anti-CD28 x anti-CEACAM5 antibody is a CEACAM5 ABD depicted in FIG. 41 and FIG. 45.

In addition to the parental CEACAM5 variable heavy and variable light domains disclosed herein, provided herein are CEACAM5 ABDs that include a variable heavy domain and/or a variable light domain that are variants of a CEACAM5 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CEACAM5 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a CEACAM5 ABD depicted in FIG. 41 and FIG. 45. In some embodiments, the changes are in a VH domain depicted in FIG. 41 and FIG. 45. In some embodiments, the changes are in a VL domain are depicted in FIG. 41 and FIG. 45. In some embodiments, the changes are in a VH and VL domain are depicted in FIG. 41 and FIG. 45. In some embodiments, the one or more amino acid changes are in the VH and/or VL framework regions (FR1, FR2, FR3, and/or FR4). In some embodiments, the one or more amino acid change(s) are in one or more of vhCDR1-3 and/or vlCDR1-3. In some embodiments, the CEACAM5 variant includes the vhCDR1-3 and/or vlCDR1-3 of any of the CEACAM5 ABDs in FIG. 41 and FIG. 45. In certain embodiments, the CEACAM5 ABD of the anti-CD28 x anti-CEACAM5 antibody is capable of binding to CEACAM5, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CEACAM5 ABD is capable of binding human CEACAM5 antigen (FIG. 2).

In one embodiment, the variant VH and/or VL domain is at least 90, 95, 97, 98 or 99% identical to the VH and/or VL of a CEACAM5 ABD as described herein, including the figures and sequence listing. In exemplary embodiments, the variant VH and/or VL domain is at least 90, 95, 97, 98 or 99% identical to the VH and/or VL of a CEACAM5 ABD depicted in FIG. 41 and FIG. 45. In some embodiments, the CEACAM5 ABD includes a VH that is at least 90, 95, 97, 98 or 99% identical to VH domain depicted in FIG. 41 and FIG. 45. In some embodiments, the CEACAM5 ABD includes a VL that is at least 90, 95, 97, 98 or 99% identical to VL domain depicted in FIG. 41 and FIG. 45. In some embodiments, the CEACAM5 ABD includes a VH and a VL that is at least 90, 95, 97, 98 or 99% identical to a VH domain and a VL domain depicted in FIG. 41 and FIG. 45. In some embodiments, the CEACAM5 ABD includes the 6 CDRs (vhCDR1-3 and vlCDR1-3) of any of the CEACAM5 ABDs of FIG. 41 and FIG. 45. In certain embodiments, the CEACAM5 ABD of the anti-CD28 x anti-CEACAM5 antibody is capable of binding to the CEACAM5, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CEACAM5 ABD is capable of binding human CEACAM5 antigen (FIG. 2).

In some embodiments, the anti-CD28 x anti-CEACAM5 antibody is a bivalent antibody (e.g., 1+1 Fab-scFv-Fc format antibody) that includes one CEACAM5 binding domain. In other embodiments, the anti-CD28 x anti-CEACAM5 antibody is a trivalent antibody (e.g., 2+1 mAb-scFv, 2+1 Fab2.scFv-Fc, and 2+1 Fab2-Fc x scFv-Fc format antibodies) that includes two CEACAM5 binding domains.

In another aspect, provided herein is a CEACAM5 binding domain that competes with any of the PD-L1 binding domains disclosed herein for binding to human CEACAM5.

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10⁻²⁰ amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-CD28 x anti-CEACAM5 antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers.

As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3 and 8.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A- monomer B” pairs that rely on “knobs and holes.” In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants.” These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3 and 8. Such “skew” variants include, but are not limited to: S364K/E357Q: L368D/K370S; L368D/K370S: S364K; L368E/K370S: S364K; T411T/E360E/Q362E: D401K; L368D/K370S: S364K/E357L; K370S: S364K/E357Q (EU numbering).

In exemplary embodiments, the heterodimeric antibody includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) “skew” variant amino acid substitution set (EU numbering). In an exemplary embodiment, the heterodimeric antibody includes a “S364K/E357Q: L368D/K370S” amino acid substitution set. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotopic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies. 2. Purification Variants

In some embodiments, the heterodimeric antibody includes purification variants that advantageously allow for the separation of heterodimeric proteins (e.g., anti-CD28 x anti-CEACAM5 bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins.

Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants.

Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIGS. 3 and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab2-scFv-Fc, 1+1 CLC and 2+1 CLC formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position.

Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 3 and 4, and FIG. 30 of US Publ. App. No. 2016/0355608, all of which are herein incorporated by reference in its entirety and specifically for the disclosure of pI variants. Preferred combinations of pI variants are shown in FIGS. 3 and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄(SEQ ID NO: 24). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 8 and the other monomer has a charged linker (either in the form of a charged scFv linker because that monomer comprises an scFv or a charged domain linker, as the format dictates, which can be selected from those depicted in FIG. 6).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-CD28 x anti-CEACAM5 antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding. 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point. 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared. 5. pI Variants that also confer better FcRn in vivo binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood.

In mice, Dall′ Acqua et al. showed that Fc mutants with increased FcRn binding at pH 6 and pH 7.4 actually had reduced serum concentrations and the same half-life as wild-type Fc (Dall′ Acqua et al. 2002, J. Immunol. 169:5171-5180, entirely incorporated by reference).

The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein. 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. Nos. 8,188,321 (particularly FIG. 41) and 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 2591, 428L/434S, 2591/308F, 4361/428L, 4361 or V/434S, 436V/428L, 259I/308F/428L, and M252Y/S254T/T256E. Such modification may be included in one or both Fc domains of the subject antibody. 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred aspects utilizing ablation variants selected from the group consisting of. L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition.” In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 mAb-Fc, 2+1 Fab2-scFv-Fc, and 2+1 Fab2-Fc x scFv-Fc format antibodies are included in FIG. 8. In some embodiments, the heterodimeric antibody includes a combination of variants as depicted in FIG. 8.

### G. Useful Antibody Formats

As will be appreciated by those in the art and discussed more fully below, the heterodimeric bispecific antibodies provided herein can take on several different configurations as generally depicted in FIG. 14.

As will be appreciated by those in the art, the heterodimeric formats of the invention can have different valences as well as be bispecific. That is, heterodimeric antibodies of the invention can be bivalent and bispecific, or trivalent and bispecific, wherein the first antigen is bound by two binding domains and the second antigen by a second binding domain. As is outlined herein, when CD28 is one of the target antigens, it is preferable that the CD28 is bound only monovalently.

The present invention utilizes CD28 binding domains in combination with CEACAM5 binding domains. As will be appreciated by those in the art, any collection of anti-CD28 CDRs, anti-CD28 variable light and variable heavy domains, Fabs and scFvs as depicted in any of the Figures (see particularly FIGS. 15-18, 21 and 48) or variants thereof can be used. Similarly, any of the CEACAM5 antigen binding domains can be used, whether CDRs, variable light and variable heavy domains, Fabs and scFvs as depicted in any of the figures (e.g., FIG. 41 and FIG. 45) or variants thereof can be used, optionally and independently combined in any combination.

1. 1+1 Fab-scFv-Fc format

One heterodimeric antibody format that finds particular use in subject anti-CD28 x anti-CEACAM5 antibodies provided herein is the “1+1 Fab-scFv-Fc” or “bottle opener” format as shown in FIG. 14A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv,” as defined below) and a second Fc domain.

The scFv includes a second variable heavy domain (VH2) and a second variable light domain (VL2), wherein the VH2 is attached to the VL2 using an scFv linker that can be charged (see, e.g., FIG. 6). The scFv is attached to the heavy chain using a domain linker (see, e.g., FIG. 7). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a CD28 binding domain and the other binding domain is a CEACAM5 binding domain. In some embodiments where the 1+1 Fab-scFv-Fc, it is the scFv that binds to the CD28, and the Fab that binds CEACAM5. Exemplary anti-CD28 x anti-CEACAM5 bispecific antibodies in the 1+1 Fab-scFv-Fc format are depicted in FIG. 24.

In some embodiments, the first and second Fc domains of the 1+1 Fab-scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody includes a combination of amino acid modifications as depicted in FIG. 8. In such embodiments, the CH1-hinge-CH2-CH3 of the first monomer comprises amino acid variants L368D/K370S/N208D/Q295E/N384D/Q418E/N421D/E233P/L234V/L235A/G236del/S267 K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 6). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody, the first Fc domain includes heterodimerization skew variants L368D/K370S and the second Fc domain includes heterodimerization skew variants S364K/E357Q; each of the first and second Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker (SEQ ID NO: 24). In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in FIG. 6).

Any suitable CD28 binding domain can be included in subject 1+1 Fab-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and IA7[CD28]_H1.106_L1 [S-S](FIGS. 15, 18, 21 and 65). Additional VH and VL sequences of exemplary CD28 binding domains that can be used in the subject 1+1 Fab-scFv-Fc format antibody are depicted in FIGS. 15, 16, 17, and 82.

In some embodiments of the 1+1 Fab-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 15, 17, or 82 or a variant thereof.

In some embodiments of the 1+1 Fab-scFv-Fc format, the anti-CD28 ABD has a VH and VL domain selected from the following:
(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or
(i) a VH having an amino acid sequence of a VH depicted in FIG. 15 or 16 or a variant thereof; and (ii) a VL having an amino acid sequence of the VL of a VL depicted in FIG. 15, 17, or 82 or a variant thereof.

Any suitable CEACAM5 binding domain can be included in subject 1+1 Fab-scFv-Fc format antibody, including any of the CEACAM5 binding domains provided herein. Exemplary CEACAM5 binding domains that can be used in the subject 1+1 Fab-scFv-Fc format antibody are depicted in FIG. 41 and FIG. 45.

In some embodiments of the 1+1 Fab-scFv-Fc format, the CEACAM5 ABD has (i) a VH having an amino acid sequence of a VH of any one of the CEACAM5 binding domains depicted in FIG. 41 and FIG. 45 or a variant thereof; and (ii) a VL having an amino acid sequence of the VL of the CEACAM5 binding domain depicted in FIG. 41 and FIG. 45 or a variant thereof.

FIG. 9 shows some exemplary Fc domain sequences that are useful in the 1 +1 Fab-scFv-Fc format antibodies. The “monomer 1” sequences depicted in FIG. 9 typically refer to the Fc domain of the “Fab-Fc heavy chain” and the “monomer 2” sequences refer to the Fc domain of the “scFv-Fc heavy chain.” In addition, FIGS. 11 and 12 provide exemplary CH1-hinge domains, CH1 domains, and hinge domains that can be included in the first or second monomer of the 1+1 Fab-scFv-Fc format. Further, FIG. 13 provides useful CL sequences that can be used with this format.

2. 2+1 mAb-scFv Format

One heterodimeric antibody format that finds particular use in the subject bispecific anti-CD28 x anti-CEACAM5 antibodies is the 2+1 mAb-scFv format shown in FIG. 14E. This antibody format includes three antigen binding domains: two Fab portions and an scFv that is attached to the C-terminal of one of the heavy chains. In some embodiments of this format, the Fab portions each bind CEACAM5, in this case, human CEACAM5 and the “extra” scFv domain binds CD28. That is, this mAb-scFv format is a trivalent antibody.

In these embodiments, the first chain or monomer comprises, from N- to C-terminal, VH1-CHT-hinge-CH2-CH3, the second monomer comprises, from N- to C-terminal, VH1-CHT-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In embodiments, this format includes two identical light chains (VL1-CL). In this format, the VHTs are each a first variable heavy (VH) domain, the VLTs are each a first variable light (VL) domain, the VH2 is a second variable heavy domain, and the VL2 is a second variable light domain. In some embodiments, the format includes two identical common light chains (each VL1-CL), wherein each of the common light chains associates with one of the VH1-CHT of the first and second monomers to form two identical Fabs (i.e., first antigen binding domains). In some embodiments, the scFv is the second antigen binding domain. In some embodiments, the first ABDs bind to human CEACAM5, and the second ABD binds human CD28.

In some embodiments, the first and second Fc domains of the 2+1 mAb-scFv format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 6). In some embodiments, the 2+1 mAb-scFv format antibody provided herein includes FcRn variants M428L/N434S, wherein numbering is according to EU numbering.

In exemplary embodiments the 2+1 mAb-scFv format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker (SEQ ID NO: 24). In some embodiments, 2+1 mAb-scFv format antibody provided herein includes FcRn variants M428L/N434S, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind CEACAM5. Any suitable CEACAM5 binding domain and CD28 binding domain can be included in the 2 +1 mAb-scFv format antibody, including any of the CEACAM5 binding domains and CD28 binding domains and related VHs and VLs provided herein or a variant thereof (see, e.g., FIGS. 15-18, 21 and 48).

In some embodiments of the 2+1 mAb-scFv format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 15, 17, or 82 or a variant thereof.

In some embodiments of the 2+1 mAb-scFv format, the anti-CD28 ABD has a VH and VL domain selected from the following:
(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or
(i) a VH having an amino acid sequence of a VH depicted in FIG. 15 or 16 or a variant thereof; and (ii) a VL having an amino acid sequence of the VL of a VL depicted in FIG. 15, 17, or 82 or a variant thereof.

In some embodiments of the 2+1 mAb-scFv format, each of the CEACAM5 ABDs has (i) a VH having an amino acid sequence of a VH of any one of the CEACAM5 binding domains depicted in FIG. 41 and FIG. 45 or a variant thereof; and (ii) a VL having an amino acid sequence of the VL of the CEACAM5 binding domain depicted in FIG. 41 and FIG. 45 or a variant thereof.

FIG. 10 shows some exemplary Fc domain sequences that are useful in the 2+1 mAb-scFv format antibodies. In addition, FIGS. 11 and 12 provide exemplary CH1-hinge domains, CH1 domains, and hinge domains that can be included in the first or second monomer of the 2+1 mAb-scFv format. Further, FIG. 13 provides useful CL sequences that can be used with this format.

3. 2+1 Fab2-scFv-Fc format

One heterodimeric antibody format that finds particular use in subject anti-CD28 x anti-CEACAM5 antibodies provided herein is the 2+1 Fab2-scFv-Fc format (also referred to as “central-scFv format”) shown in FIG. 14B. This antibody format includes three antigen binding domains: two Fab portions and an scFv that is inserted between the VH-CH1 and CH2-CH3 regions of one of the monomers. In some embodiments of this format, the Fab portions each bind CEACAM5 and the “extra” scFv domain binds CD28. In some embodiments, the 2+1 Fab2-scFv-Fc format antibody is a trivalent antibody.

In some embodiments of the 2+1 Fab2-scFv-Fc format, a first monomer includes a standard heavy chain (i.e., VH1-CHT-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VHT-CHT-[optional linker]-VH2-scFv linker-VH2-[optional linker]-CH2-CH3, or the opposite orientation for the scFv, VH1-CHT-[optional linker]-VL2-scFv linker-VH2-[optional linker]-CH2-CH3). The optional linkers can be any suitable peptide linkers, including, for example, the domain linkers included in FIG. 7. This embodiment further utilizes a common light chain that includes a variable light domain (VL1) and a constant light domain (CL). In some embodiments, the format includes two identical common light chains (each VL1-CL), wherein each of the common light chains associates with one of the VH1-CHT of the first and second monomers to form two identical Fabs. In some embodiments, the identical Fabs each bind CEACAM5, and the scFv binds CD28. As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-scFv-Fc format antibody provided herein includes a charged scFv linker (including those shown in FIG. 6). In some embodiments, the 2+1 Fab2-scFv-Fc format antibody provided herein includes FcRn variants M428L/N434S, wherein numbering is according to EU numbering.

In exemplary embodiments 2+1 Fab2-scFv-Fc format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CHT-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO: 24). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind CEACAM5.

Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof 1A7[CD28]_H1L1, 1A7[CD28]_H1.1_L1, 1A7[CD28]_H1_L1.71, 1A7[CD28]_H1.1_L1.71, 1A7[CD28]H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65).

In some embodiments of the 2+1 Fab2-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 15, 17, or 82 or a variant thereof.

In some embodiments of the 2+1 Fab2-scFv-Fc format, the anti-CD28 ABD has a VH and VL domain selected from the following:
(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or
(i) a VH having an amino acid sequence of a VH depicted in FIG. 15 or 16 or a variant thereof; and (ii) a VL having an amino acid sequence of the VL of a VL depicted in FIG. 15, 17, or 82 or a variant thereof.

In some embodiments, the VH1 of the first and second monomer and the VL1 of the common light chain of the 2+1 Fab2-scFv-Fc format antibody each form a binding domain that binds CEACAM5. Any suitable CEACAM5 binding domain can be included in subject 2+1 Fab2-scFv-Fc format antibody, including any of the CEACAM5 binding domains provided herein. Exemplary CEACAM5 binding domains that can be used in the subject 2+1 Fab2-scFv-Fc format antibody are depicted in FIG. 41 and FIG. 45.

In some embodiments of 2+1 Fab2-scFv-Fc format, the CEACAM5 ABDs each include (i) a VH having an amino acid sequence of a VH of any one of the CEACAM5 binding domains depicted in FIG. 41 and FIG. 45 or a variant thereof; and (ii) a VL having an amino acid sequence of the VL of the CEACAM5 binding domain depicted in FIG. 41 and FIG. 45 or a variant thereof.

FIG. 9 shows some exemplary Fc domain sequences that are useful in the 2 +1 Fab2-scFv-Fc format antibodies. In addition, FIGS. 11 and 12 provide exemplary CH1-hinge domains, CH1 domains, and hinge domains that can be included in the first or second monomer of the 2+1 Fab2-scFv-Fc format. Further, FIG. 13 provides useful CL sequences that can be used with this format.

4. 2+1 Fab2-Fc x scFv-Fc format

One heterodimeric antibody format that finds particular use in the subject anti-anti-CD28 x anti-CEACAM5 antibodies provided herein is the 2+1 Fab2-Fc x scFv-Fc format (also referred to as “stacked bottle opener”) shown in FIG. 14F. This format includes a first monomer, a second monomer and a common light chain. The first monomer includes, from N- to C-terminus: a VH1-CH1-domain linker-VH1-CH1-hinge-CH2-CH3, wherein the VHls are each a first variable heavy domain and the CH2-CH3 is a first variant Fc domain.

The domain linker can be any useful domain linker (see, e.g., FIG. 7). The second monomer includes a single-chain Fv (“scFv”) covalently attached to a second variant Fc domain by a domain linker (scFv-domain linker-CH2-CH3). The common light chain include a VL1-CL wherein the VL1 is a first variable light domain. The scFv of the second monomer includes a second variable heavy domain (VH2) attached to a second variable light domain (VL2) by an scFv linker. In some embodiments, this format includes two identical common light chains (VL1-CL). In this embodiment, the two VH1-CHTs of the first monomer each interact with a common light chain to form two identical Fabs that are first antigen binding domains, and the VH2 and VL2 form a second antigen binding domain. In some embodiments, each of the first antigen binding domains are CEACAM5 binding domains, and the second antigen binding domain is a CD28 binding domain. As for many of the embodiments herein, these constructs can include skew variants, pI variants, ablation variants, additional Fc variants, etc. as desired and described herein.

In some embodiments, the first and second Fe domains of the 2+1 Fab2-Fc x scFv-Fc format antibody are variant Fe domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the 2+1 Fab2-Fc x scFv-Fc format antibody provided herein includes a charged scFv linker (including those shown in FIG. 6). In some embodiments, the 2+1 Fab2-scFv-Fc format antibody provided herein includes FcRn variants M428L/N434S, wherein numbering is according to EU numbering.

In exemplary embodiments, the 2+1 Fab2-Fc x scFv-Fc format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO:24). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-Fc x scFv-Fc format antibody is a CD28 binding and the VH1 of the first and second monomer and the VL1 of the common light chain each form binding domains that bind CEACAM5.

Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]H1L1, 341VL36[CD28]_H1L1, 281VL4[CD28]_H1L1, HuTN228[CD28]_H1L1, PV1[CD28]H0L0, m9.3[CD28]_H0L0, hu9.3[CD28]_H1L1, 9G2[CD28]_H0L0, 9G2[CD28]_H1L1, 2F10A3.140[CD28]_H1L1, TN228[CD28]_H4L2, IA7[CD28]_H1.1_L1 [S-S], IA7[CD28]_H1_L1.71 [S-S], IA7[CD28]_H1.1_L1.71 [S-S], IA7[CD28]_H1.14_L1 [S-S], IA7[CD28]_H1.14_L1.71 [S-S], IA7[CD28]_H1.129_L1.71 [S-S], 1A7[CD28]_H1.106_L1.71 [S-S], 1A7[CD28]_H1.129_L1 [S-S], and 1A7[CD28]_H1.106_L1 [S-S](FIGS. 15, 18, 21 and 65).

In some embodiments of the 2+1 Fab2-Fc x scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 15, 18, 21 and 65 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 15, 18, 21 and 65 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 15 or 16 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 15, 17, or 82 or a variant thereof.

In some embodiments of the 2+1 Fab2-Fc x scFv-Fc, the anti-CD28 ABD has a VH and VL domain selected from the following:
(i) a VH having an amino acid sequence of a VH of any of the CD28 binding domains depicted in FIGS. 15, 18, 21 and 65 or a variant thereof; and (ii) a VL having an amino acid sequence of the VL of the CD28 binding domain depicted in FIGS. 15, 18, 21 and 65 or a variant thereof; or
(i) a VH having an amino acid sequence of a VH depicted in FIG. 15 or 16 or a variant thereof; and (ii) a VL having an amino acid sequence of the VL of a VL depicted in FIG. 15, 17, or 82 or a variant thereof.

In some embodiments, the VH1 of the first and second monomer and the VL1 of the common light chain of the 2+1 Fab2-Fc x scFv-Fc format antibody each form a binding domain that binds CEACAM5. Any suitable CEACAM5 binding domain can be included in subject 2+1 Fab2-Fc x scFv-Fc format antibody, including any of the CEACAM5 binding domains provided herein. Exemplary CEACAM5 binding domains that can be used in the subject 2+1 Fab2-Fc x scFv-Fc format antibody are depicted in FIG. 41 and FIG. 45.

In some embodiments of 2+1 Fab2-Fc x scFv-Fc format, the CEACAM5 ABDs each include (i) a VH having an amino acid sequence of a VH of any one of the CEACAM5 binding domains depicted in FIG. 41 and FIG. 45 or a variant thereof.

FIG. 9 shows some exemplary Fc domain sequences that are useful in 2+1 Fab2-Fc x scFv-Fc format antibodies. In addition, FIGS. 11 and 12 provide exemplary CH1-hinge domains, CH1 domains, and hinge domains that can be included in the first or second monomer of the 2+1 Fab2-Fc x scFv-Fc format. Further, FIG. 13 provides useful CL sequences that can be used with this format. 5. 1+1 CLC Format

One heterodimeric antibody format that finds particular use in subject anti-CD28 x anti-CEACAM5 antibodies provided herein is the “1+1 Common Light Chain” or “1+1 CLC” format, which is depicted in FIG. 14C. The 1+1 CLC format antibody includes a first monomer that includes a VH1-CHT-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen binding specificity; and the VL pairs with the VH2 to form a second binding domain with a second antigen binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIGS. 3 and 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267 K, and the second Fc domain comprises amino acid variants S364K/E357Q/E233P/L234V/L235A/G236del/S267K, wherein numbering is according to EU numbering.

In some embodiments, the 1+1 CLC format antibody provided herein further includes FcRn variants M428L/N434S, wherein numbering is according to EU numbering.

In some embodiments, one of the first binding domain or the second binding domain binds CD28 and the other binding domain binds CEACAM5. Any suitable CD28 binding domain and CEACAM5 domain can be included in subject 1+1 CLC format antibody, including any of the CD28 binding domains and CEACAM5 binding domains provided herein or a variant thereof (see, e.g., FIGS. 15-18, 21, 41, 45, 48, and 82). 6. 2+1 CLC Format

Another heterodimeric antibody format that finds particular use in subject CD28 x anti-CEACAM5 antibodies provided herein is the “2+1 Common Light Chain” or “2+1 CLC” format, which is depicted in FIG. 14D. The 2+1 CLC format includes a first monomer that includes a VH1-CH1-linker-VH1-CH1-hinge-CH2-CH3, wherein the VHls are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VH1s of the first monomer to form two first binding domains, each with a first antigen binding specificity; and the VL pairs with the VH2 to form a second binding domain with a second antigen binding specificity. The linker of the first monomer can be any suitable linker, including any one of the domain linkers or combinations thereof described in FIG. 7. In some embodiments, the 2+1 CLC format antibody is a trivalent antibody.

In some embodiments, the first and second Fe domains of the 2+1 CLC format are variant Fe domains that include heterodimerization skew variants (e.g., a set of amino acid substitutions as shown in FIGS. 3 and 8). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CHT-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267 K, and the first Fc domain comprises amino acid variants S364K/E357Q/E233P/L234V/L235A/G236del/S267K, wherein numbering is according to EU numbering.

In some embodiments, each of the two first binding domains binds a tumor CEACAM5 and the second binding domain binds CD28. Any suitable CD28 binding domain and CEACAM5 domain can be included in the subject 2+1 CLC format antibody, including any of the CD28 binding domains and CEACAM5 binding domains provided herein or a variant thereof (see, e.g., FIGS. 15-18, 21, 41, 45, 48, and 82). 7. Dual scFv formats

One heterodimeric antibody format that finds particular use in the subject bispecific anti-CD28 x anti-CEACAM5 antibodies is the dual scFv format, as are known in the art and shown in FIG. 14G. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation.

In this case, all ABDs are in the scFv format. Any suitable CEACAM5 binding domain and CD28 binding domain can be included in anti-CD28 x anti-CEACAM5 antibody in the dual scFv format, including any of the CEACAM5 binding domains and CD28 binding domains provided herein.

In addition, the Fc domains of the dual scFv format comprise skew variants (e.g. a set of amino acid substitutions as shown in FIGS. 3 and 8, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 5), optionally charged scFv linkers (including those shown in FIG. 6) and the heavy chain comprises pI variants (including those shown in FIG. 4).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable CEACAM5 binding domain and CD28 binding domain can be included in the dual scFv format, including any of the CEACAM5 binding domains and CD28 binding domains and related VHs and VLs provided herein or a variant thereof (see, e.g., FIGS. 15-18, 21, 41, 45, 48, and 82).

8. One-armed central-scFv

One heterodimeric antibody format that finds particular use in subject anti-CD28 x anti-CEACAM5 antibodies provided herein is the one-armed central-scFv format shown in FIG. 14K. In this embodiment, one monomer comprises just an Fc domain, while the other monomer includes a Fab domain (a first antigen binding domain), a scFv domain (a second antigen binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain, comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable CEACAM5 binding domain and CD28 binding domain can be included in the one-armed central-scFv format, including any of the CEACAM5 binding domains and CD28 binding domains and related VHs and VLs provided herein or a variant thereof (see, e.g., FIGS. 15-18, 21, 41, 45, 48, and 82).

9. One-armed scFv-mAb format

One heterodimeric antibody format that finds particular use in subject anti-CD28 x anti-CEACAM5 antibodies provided herein is the one-armed mAb-scFv format shown in FIG. 14H. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds CEACAM5. As for many of the embodiments herein, these constructs include skew variants, pI variants, ablation variants, additional Fc variants, etc. as desired and described herein.

Any suitable CEACAM5 binding domain and CD28 binding domain can be included in the one-armed scFv-mAb format antibody, including any of the CEACAM5 binding domains and CD28 binding domains and related VHs and VLs provided herein or a variant thereof (see, e.g., FIGS. 15-18, 21, 41, 45, 48, and 82).

10. scFv-mAb format

One heterodimeric antibody format that finds particular use in subject anti-CD28 x anti-CEACAM5 antibodies provided herein is the mAb-scFv format shown in FIG. 141. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vhl-scFv linker-vll-[optional domain linker]- vh2-CH1-hinge-CH2-CH3) or (with the scFv in the opposite orientation) ((vll-scFv linker-vhl-[optional domain linker]-vh2-CHT-hinge-CH2-CH3)). The second monomer comprises a heavy chain VH2-CH1-hinge-CH2-CH3. This embodiment further utilizes a common light chain, comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable CEACAM5 binding domain and CD28 binding domain can be included in the scFv-mAb antibody format, including any of the CEACAM5 binding domains and CD28 binding domains and related VHs and VLs provided herein or a variant thereof (see, e.g., FIGS. 15-18, 21, 41, 45, 48, and 82).

11. mAb-Fv format

One heterodimeric antibody format that finds particular use in subject anti-CD28 x anti-CEACAM5 antibodies provided herein is the mAb-Fv format (FIG. 14L). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind CD28 and the “extra” Fv domain binds CEACAM5.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vhl-CHT-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vhl-CHT-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

Any suitable CEACAM5 binding domain and CD28 binding domain can be included in the mAb-Fv format, including any of the CEACAM5 binding domains and CD28 binding domains and related VHs and VLs provided herein or a variant thereof (see, e.g., FIGS. 15-18, 21, 41, 45 and 48).

12. Central-Fv format

One heterodimeric antibody format that finds particular use in subject anti-CD28 x anti-CEACAM5 antibodies provided herein is the central-Fv format shown in FIG. 14M. In this embodiment, the format relies on the use of an inserted Fv domain thus forming an “extra” third antigen binding domain, wherein the Fab portions of the two monomers bind CEACAM5 and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vhl-CH1-[optional linker]-vl2-hinge-CH2-CH3). The other monomer comprises a first heavy chain comprising a first variable heavy domain, a CH1 domain and Fc domain and an additional variable heavy domain (vhl-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain, comprising a variable light domain and a constant light domain, that associates with the heavy chains to form two identical Fabs that each bind CEACAM5. The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable CEACAM5 binding domain and CD28 binding domain can be included in the central-Fv format, including any of the CEACAM5 binding domains and CD28 binding domains and related VHs and VLs provided herein or a variant thereof (see, e.g., FIGS. 15-18, 21, 41, 45, 48, and 82). 13. Non-heterodimeric bispecific antibodies

As will be appreciated by those in the art, the anti-CD28 x anti-CEACAM5 antibodies provided herein can also be included in non-heterodimeric bispecific formats (see FIG. 14J). In this format, the anti-CD28 x anti-CEACAM5 includes: 1) a first monomer comprising a VH1-CHT-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds CEACAM5.

Any suitable CEACAM5 binding domain and CD28 binding domain can be included in anti-CD28 x anti-CEACAM5 antibody in the non-heterodimeric bispecific antibody format, including any of the CEACAM5 binding domains and CD28 binding domains and related VHs and VLs provided herein or a variant thereof (see, e.g., FIGS. 15-18, 21, 41, 45, 48, and 82).

### 14. Trident Format

In some embodiments, the anti-CD28 x anti-CEACAM5 antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 14N. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 14N, the second and third ABDs bind the same antigen.

Any suitable CEACAM5 binding domain and CD28 binding domain can be included in the trident format, including any of the CEACAM5 binding domains and CD28 binding domains and related VHs and VLs provided herein or a variant thereof (see, e.g., FIGS. 15-18, 21, 41, 45, 48, and 82).

### VI. Nucleic Acids

In another aspect, provided herein are nucleic acid compositions encoding the anti-CD28 x anti-STEAP1 antibodies provided herein. Anucleic acid composition may refer to one or multiple polynucleotides.

As will be appreciated by those in the art, the nucleic acid compositions will depend on the format and scaffold of the heterodimeric protein. Thus, for example, when the format requires three amino acid sequences, such as for the 1+1 Fab-scFv-Fc, 2+1 mAb-scFv, 2+1 Fab2-scFv-Fc, and 2+1 Fab2-Fc x scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer: second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

VII. Biological and Biochemical Functionality of the anti-CD28 x anti-STEAP1 Antibodies

Generally the anti-CD28 x anti-STEAP1 antibodies described herein are administered to patients with a STEAP1 -associated cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Embodiments of the invention relate to a pharmaceutical composition comprising any one of the anti-CD28 x anti-STEAP1 antibodies described herein and a pharmaceutically acceptable carrier. Formulations of the anti-CD28 x anti-STEAP1 antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### VIII. Treatments

Once made, the compositions of the invention find use in a number of oncology applications, by treating cancer, generally by enhancing immune responses (e.g., T cell activation and proliferation), particularly when used with anti-cancer therapies such as anti-tumor bispecific antibodies. In some embodiments, the antibodies provided herein enhance immune responses (e.g., T cell activation and proliferation) by providing agonistic co-stimulation of T cells in the microenvironment of tumors expressing STEAP1 . In some embodiments, the antibodies provided herein enhance immune responses (e.g., T cell activation and proliferation) by providing agonistic co-stimulation of T cells in the microenvironment of tumors expressing CEACAM5.

In some embodiments, the anti-CD28 x anti-STEAP1 bispecific antibodies provided herein are administered with an anti-tumor therapy including, for example, anti-tumor-associated antigen (TAA) bispecific antibodies. In some embodiments, the anti-CD28 x anti-CEACAM5 bispecific antibodies provided herein are administered with an anti-tumor therapy including, for example, anti-tumor-associated antigen (TAA) bispecific antibodies.

A. Anti-CD28 x anti-STEAP1 /anti-STEAP1 bispecific antibody

In some embodiments, the anti-CD28 x anti-STEAP1 antibodies provided herein are administered with an anti-STEAP1 bispecific antibody that is a T-cell engaging bispecific antibody, such as those that bind to human CD3.

In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 23A). In contrast, only the first signal is provided in treatment with CD3 bispecific antibodies that target a tumor-associated antigen (TAA) (i.e., anti-CD3 x anti-STEAP1 bispecific antibodies).

Without being bound by any particular theory of operation, it is believed that the anti-CD28 x anti-STEAP1 bispecific antibodies provided herein can enhance the anti-tumor response of an anti-CD3 x anti-STEAP1 bispecific antibody by CD28 costimulation (see FIG. 23B). Thus, in one aspect, provided herein are methods of methods of treating a STEAP1 -associated cancer in a patient by administering the patient an anti-CD3 x anti-STEAP1 bispecific antibody and an anti-CD28 x anti-STEAP1 bispecific antibody provided herein.

Anti-CD3 x anti-STEAP1 antibodies that are useful for providing “signal 1” in combination with the subject anti-CD28 x anti-STEAP1 antibodies include those with any of the CD3 binding domains and STEAP1 binding domains provided herein (see, e.g., FIGS. 22 and 25). Suitable antibody formats for such anti-CD3 x anti-STEAP1 antibodies, include, but are not limited to the antibody formats described herein (see, e.g., FIG. 14). In some embodiments, the anti-CD3 x anti-STEAP1 antibody and the anti-CD28 x anti-STEAP1 antibody used in combination bind to the same STEAP1 epitope. In some embodiments, the anti-CD3 x anti-STEAP1 antibody and the anti-CD28 x anti-STEAP1 antibody used in combination bind to different STEAP1 epitopes.

B. Anti-CD28 x anti-CEACAM5/anti-CEACAM5 bispecific antibody

In some embodiments, the anti-CD28 x anti-CEACAM5 antibodies provided herein are administered with an anti-CEACAM5 bispecific antibody that is a T-cell engaging bispecific antibody, such as those that bind to human CD3.

In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 23A). In contrast, only the first signal is provided in treatment with CD3 bispecific antibodies that target a tumor-associated antigen (TAA) (i.e., anti-CD3 x anti-CEACAM5 bispecific antibodies).

Without being bound by any particular theory of operation, it is believed that the anti-CD28 x anti-CEACAM5 bispecific antibodies provided herein can enhance the anti-tumor response of an anti-CD3 x anti-CEACAM5 bispecific antibody by CD28 co-stimulation (see FIG. 23B). Thus, in one aspect, provided herein are methods of methods of treating a CEACAM5-associated cancer in a patient by administering the patient an anti-CD3 x anti-CEACAM5 bispecific antibody and an anti-CD28 x anti-CEACAM5 bispecific antibody provided herein.

Anti-CD3 x anti-CEACAM5 antibodies that are useful for providing “signal 1” in combination with the subject anti-CD28 x anti-CEACAM5 antibodies include those with any of the CD3 binding domains and CEACAM5 binding domains provided herein (see, e.g., FIGS. 41, 45 and 25). Suitable antibody formats for such anti-CD3 x anti-CEACAM5 antibodies, include, but are not limited to the antibody formats described herein (see, e.g., FIG. 14). In some embodiments, the anti-CD3 x anti-CEACAM5 antibody and the anti-CD28 x anti-CEACAM5 antibody used in combination bind to the same CEACAM5 epitope. In some embodiments, the anti-CD3 x anti-CEACAM5 antibody and the anti-CD28 x anti-CEACAM5 antibody used in combination bind to different CEACAM5 epitopes.

C. Administrative modalities

The antibodies provided herein administered to a subject, in accord with known methods, such as intravenous administration as a bolus or by continuous infusion over a period of time.

D. Treatment modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

### EXAMPLES

### Example 1: CD28 Binding Domains

Sequences for human, mouse, and cynomolgus CD28 are depicted in FIG. 1 and are useful for the development of cross-reactive CD28 antigen binding domains for ease of clinical development. 1A: Novel CD28 Binding Domains

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28.

1A(a): Phage-derived clone IA7

It should be noted that this phage library utilized a human germline VL with diversity introduced into the LCDR3. The amino acid sequences for exemplary phage-derived clone IA7 are depicted in FIG. 15.

Towards optimization of CD28 bsAbs, numerous 1A7 affinity variants were developed by engineering VH variants (illustrative sequences as depicted in FIG. 16 and as SEQ ID NOs: XXX-YYY), VL variants (illustrative sequences as depicted in FIG. 17 and as SEQ ID NOs: XXX-YYY), consensus sequences for which are depicted in FIG. 19, and combinations thereof (illustrative sequences for which are depicted in FIG. 18). Monovalent affinity of illustrative variants, in the context of scFvs, are depicted in FIG. 20.

Surprisingly, it was found that the variable light domain of clone IA7 differed from parental germline IGKV1-39 VL by only a single amino acid in the LCDR3; however, despite there being only a single amino acid difference, the VH of 1A7 does not pair well with the parental germline VL resulting in diminished binding. For example, 1A7_H1.14_L1 showed an affinity of −200 nM, while IA7_H1.14_IGKV1-39 showed at affinity of>1 pM.

However, it is important that the IA7 VHs pair with IGKV1-39 so that they may be used in common light chain bispecific formats and paired with other binding domains utilizing the IGKV1-39 variable light domain. Accordingly, affinity-optimization phage library was generated with focus on substitutions only in the variable heavy domain of 1A7. Amino acid sequences for exemplary affinity-optimized IA7 variable domains for pairing with IGKV1-39 are depicted in FIG. 46 and SEQ ID Nos: XXX-YYY. Monovalent KD of affinity-optimized 1A7 Fab variants were determined via Octet, data for which are depicted in FIG. 47.

After identifying these additional 1A7 VH variants, favorite variants were paired with the original 1A7_L1 and L1.71 VLs to generate further affinity variants (in the context of scFvs). KD of these additional variants are depicted in FIG. 64. Finally, to stabilize formats comprising scFvs, disulfide-stabilized scFvs may be used. This may potentially improve behavior in high concentration formulations, boost thermal stability, and prevent Fab-LC intercalation. Disulfide-stabilized scFvs may be achieved by including cysteine substitutions in the VH and VL so that a disulfide bond is formed between the two cysteines. Illustrative such disulfide-stabilized scFvs are depicted in FIG. 65, and KD are depicted in FIG. 64. Additionally, as depicted in FIG. 63, disulfide stabilization improved melting temperature by 5.7° C.

### 1B: Additional CD28 Binding Domains

Sequences for additional CD28 binding domains which may find use in the STEAP1 x CD28 bsAbs and CEACAM5 x CD28 bsAbs of the invention are depicted in FIG. 21.

### Example 2: STEAP1 Binding Domains

Sequences for human and mouse STEAP1 are depicted in FIG. 2 and are useful for the development of cross-reactive STEAP1 antigen binding domains for ease of clinical development. Sequences for STEAP1 binding domains which may find use in the STEAP1 x CD28 bsAbs of the invention are depicted in FIG. 22, FIG. 31, and FIG. 32.

2A: Humanization & optimization of STEAP-A binding domains

The variable regions of a murine anti-STEAP1 clone STEAP-A were humanized using string content optimization (see, e.g., U.S. Pat. No. 7,657,380, issued Feb. 2, 2010). Murine (H0L0) and humanized (H1L1) STEAP-A variable regions are depicted in FIG. 22. Next, the humanized STEAP-A variable heavy and variable light sequences were engineered for optimization by removing sequence liabilities such as isomerization & deamidation sites. Optimized STEAP-A variants were designated as L1.1-L1.3 for VLs and H1.1-H1.38 for VHs. Sequences for each variant VH and VL are depicted in FIGS. 31 and 32, respectively. Notably, the VH substitution designated H1.21, removed 2 isomerization sites at positions 109 and 110, substituting D109E and D110L (numbering as in Kabat), without impacting affinity. Additionally, sequences for these variants in the context of a bivalent mAb are depicted in FIG. 30. 2B: Affinity maturation of STEAP-A binding domains

Optimized humanized STEAP-A variants were then engineered in order to improve their binding affinity to STEAP-1. In the first round, 82 variant VLs were engineered as L1.4-L1.85 and 151 variant VHs were engineered as H1.39-H1.189. Variants were produced as bivalent mAbs, the sequences for which are depicted in FIG. 30. The bivalent mAbs were then screened for binding to STEAP-1 on C4-2B cells. Cells were mixed with mAbs and incubated for 1 hour on ice, washed with PBS, stained with secondary antibody for 1 hour on ice, and washed again before analyzing binding utilizing flow cytometry. Out of these hundreds of variants, 18 showed tighter or comparable binding compared to the parental clone. Variants that showed improved binding over the parental humanized STEAP-A_H1L1, such as L1.47 (having a K36R point substitution as in Kabat) and L1.56 (having a S58Y point substitution as in Kabat) were combined in various ways with other substitutions to create a second round of affinity maturation variants.

In the second round, 34 variant VLs were engineered and designated as L1.86-L1.119 and 39 variant VHs were engineered and designated as H1.190-H1.228. Bivalent mAbs from the second round of affinity maturation were also screened for binding to STEAP-1 expressed on C4-2B cells. Flow cytometry analysis was used to determine relative maximum MFI values, AUC values, and approximate EC50 values for each test article, as summarized in FIG. 34. Some combinations of substitutions, such as XENP46525, having the H1.194 (S75R/D109E/D110L as in Kabat) variable heavy in combination with the L1.87 (S26D/S49P/T69S as in Kabat) variable light resulted in weaker binding than the original H1L1, illustrating the unpredictable nature of combining substitutions. However, many other test articles, such as XENP46500, having the H1.21 (D109E/D110L as in Kabat) variable heavy and L1.115 (S28T/S49P/S58Y/T69S/N99K as in Kabat) variable light domain, showed significantly improved binding when compared to one of the strongest single point substitution binders, XENP45835, having the variable light domain L1.56 (S58Y as in Kabat). A final set of variable heavy domain substitutions were engineered and designated H1.229- H1.232. These all showed comparable binding to H1.228. Sequences for each variant VH and VL are depicted in FIGS. 31 and 32, respectively.

While the affinity maturation campaign resulted in the identification of many variable heavy and variable light domains that showed significantly improved binding to STEAP1 when compared with the parental humanized H1L1, two variable light chains found particular use in the invention: L1.100, having substitutions S28T S49P S58Y T69S (numbering as in Kabat), and L1.115, having substitutions S28T S49P S58Y T69S N99K (numbering as in Kabat). Additionally, one variable heavy chain found particular use in the invention: H1.230, having substitutions A35S S54G N55T S63V Di09E D110L. These sequences and their CDRs are depicted in FIG. 39.

### Example 3: CEACAM5 Binding Domains

Sequences for human, mouse, and cynomolgus CEACAM5 are depicted in Figure and are useful for the development of cross-reactive CEACAM5 antigen binding domains for ease of clinical development. Sequences for CEACAM5 binding domains which may find use in the CEACAM5 x CD28 bsAbs of the invention are depicted in FIG. 41 and FIG. 45.

### 2A: Humanizing and Affinity-Engineering CEA-A

The CEA-A_H0L0 binding domain was humanized using string content optimization (see, e.g., U.S., Pat. No. 7,657,380), sequences for which are depicted in FIG. 45. The parental CEA-A H0 VH had 70% humanness, and the L0 VL had 65% humanness. Humanized VH variant H1 had 84% humanness. Two humanized VL variants L1 and L2 were engineered which respectively achieved 82% and 88% humanness. Surprisingly, the humanized H1L1 and HIL2 CEA-A binding domains were decreased in affinity for CEACAM5 (H0L0=0.7-2 nM; H1L1=5-8 nM; H1L2=57-69 nM at 25° C.).

To restore affinity, the H1, L1, and L2 domains were affinity-engineered through a number of mechanisms including germlining, back mutations to parental H0L0, and removing degradation labile amino acids. Amino acid sequences for exemplary affinity-optimized humanized CEA-A variable domains are depicted in FIG. 45 and SEQ ID Nos: XXX-YYY.

One such variant, H1.7_L2.15 which included removing a potential HCDR2 deamidation and framework germlining (in each of the variable heavy and variable light domains) achieved 13 nM CEACAM5 binding affinity (86% humanness in VH; 86% humanness in VL).Finally using CEA-A H1.7_L2.15 as a new starting point, further variants were engineered to reduce cross-reactive binding to other members of the CEA family. These efforts are further described in Example X. Amino acid sequences for exemplary such sequences are depicted in FIG. 45 and SEQ ID NOs: XXX-YYY. One such variant, H1.13_L2.15 was reduced in CEACAM8 binding affinity (10890 nM vs. 3330 nM for the H1.7_L2.15 variant) while maintaining CEACAM5 binding affinity (18 nM vs. 13 nM for the H1.7_L2.15 variant).

### Example 4: Engineering STEAP1 x CD28 and CEACAM5 x CD28 bsAbs

T cells require multiple signals for complete activation and differentiation. Signal 1, promoted by recognition of a peptide-MHC (pMHC) complex by the T cell receptor (TCR), is absolutely required for T cell activation. Signal 2, which synergizes with, and amplifies Signal 1, is typically provided by the interaction of the CD28 ligands CD80 and CD86 with CD28 itself. Although CD28 engagement alone is typically inert, when combined with Signal 1 activation, it promotes additional activation, survival, and proliferative signals, including IL2 secretion (see FIG. 23). As CD80 and CD86 are only naturally expressed by professional antigen-presenting cells (APC), the extent of CD28 costimulation in the tumor setting can be highly variable. By creating this novel class of tumor-targeted CD28 bispecific antibodies, CD80/CD86 engagement of CD28 can be mimicked, providing an artificial source of Signal 2. Notably, signal can either be provided by the natural TCR:pMHC recognition of tumor cells, or it can be provided by combination of the CD28 bispecific with a CD3 bispecific (which can mimic Signal 1). With these concepts in mind, STEAP1 x CD28 and CEACAM5 x CD28 bsAbs were conceived, and a number of formats are contemplated for use, schematics for which are outlined in FIG. 14.

### 4A: 1+1 Fab-scFv-Fc Format

One exemplary format utilizing Fab domains and scFv is the 1+1 Fab-scFv-Fc format (depicted schematically in FIG. 14A) which comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain. Any number of heterodimerization approaches as is known in the art could find use in this (and other) bispecific formats, in combination with any number of approaches for purifying heterodimers from contaminating homodimers, including those depicted in FIG. 3. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.

In particular, the 1+1 Fab-scFv-Fc bsAbs may utilize Backbones 1 or 11 in FIG. 9. The backbones utilize the L368D/K370S (on the HC): S364K/E357Q (on the scFv-Fc) heterodimeric Fc variants. The HC side further includes pI variants N208D/Q295E/N384D/Q418E/N421D to increase negative charge of the heavy chain. The scFv utilizes a positively charged (GKPGS)₄ linker (SEQ ID NO: 24) between the VH and VL domains to increase positive charge of the scFv-Fc chain. Collectively, these two approaches enable easy purification of heterodimers from contaminating homodimers. The FcγR ablation variants utilized in this platform are the E233P/L234V/L235A/G236del/S267K substitutions on both the HC and the scFv-Fc monomers. In some cases, bsAbs includes the M428L/N434S half-life extension variants. Sequences for illustrative STEAP1 x CD28 bsAbs in the 1+1 Fab-scFv-Fc format are depicted in FIG. 24. Sequences for illustrative CEACAM5 x CD28 bsAbs in the 1+1 Fab-scFv-Fc format are depicted in FIG. 42. 4B: 2+1 mAb-scFv Format

Another exemplary format utilizing Fab domains and scFv is the 2+1 mAb-scFv format (depicted schematically in FIG. 14E) which comprises a first monomer comprising a first heavy chain covalently attached to a single-chain Fv (“scFv”) with a first antigen binding specificity i.e., VH-CH1-hinge-CH2-CH3-domain linker-scFv, wherein CH2-CH3 is a first heterodimeric Fc domain; a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domains having a second antigen binding specificity are formed with the two VH domains. In particular, the 2+1 Fab-scFv-Fc bsAbs may utilize backbone in FIG. 10. The backbones utilize the L368D/K370S (on the HC): S364K/E357Q (on the HC-scFv side) heterodimeric Fc variants. The HC further includes pI variants N208D/Q295E/N384D/Q418E/N421D to increase negative charge of the heavy chain. The scFv utilizes a positively charged (GKPGS)₄ linker (SEQ ID NO: 24) between the VH and VL domains to increase positive charge of the HC-scFv chain. Collectively, these two approaches enable easy purification of heterodimers from contaminating homodimers. The FcγR ablation variants utilized in this platform are the E233P/L234V/L235A/G236del/S267K substitutions on both the HC and the scFv-Fc monomers. In some cases, bsAbs includes the M428L/N434S half-life extension variants. Sequences for illustrative STEAP1 x CD28 bsAbs in the 2+1 mAb-scFv Format are depicted in FIG. 27. Sequences for illustrative CEACAM5 x CD28 bsAbs in the 2+1 mAb-scFv format are depicted in FIG. 48. 3C: 2+1 Fab2-Fc x scFv-Fc Format

Another exemplary format utilizing Fab domains and scFv is the 2+1 Fab₂-Fc x scFv-Fc format (depicted schematically in FIG. 14F) which includes a first monomer, a second monomer, and a light chain. The first monomer comprises a first VH1 domain covalently attached to a first CH1 domain covalently attached (e.g., by a domain linker) to a second VH1 domain covalently attached to a second CH1 domain covalently attached to a first heterodimeric Fc domain (e.g., VH-CH1-VH-CH1-hinge-CH2-CH3). The second monomer comprises a single-chain Fv (“scFv”) covalently attached to a second heterodimeric Fc domain complementary to the first heterodimeric Fc domain (scFv-domain linker-CH2-CH3). The first monomer, second monomer and light chain are transfected separately so that the two VH1-CH1 of the first monomer and the light chain form two antigen binding domains with the same antigen binding specificity. The scFv of the second monomer has a second antigen binding specificity. Sequences for illustrative STEAP1 x CD28 bsAbs in the 2+1 Fab2-Fc x scFv-Fc format are depicted in FIG. 28. Sequences for illustrative CEACAM5 x CD28 bsAbs in the 2+1 Fab2-Fc x scFv-Fc format are depicted in FIG. 50. 4D: 2+1 Fab2-scFv-Fc

Another exemplary format utilizing Fab domains and scFv is the 2+1 Fab₂-scFv-Fc format (depicted schematically in FIG. 14B) which comprises a first monomer comprising a heavy chain i.e., VH-CH1-hinge-CH2-CH3, wherein the CH2-CH3 is a first heterodimeric Fc domain; a second monomer comprising a VH-CHT-linker-scFv-linker-CH2-CH3, wherein the CH2-CH3 is a second heterodimeric Fc domain complementary to the first heterodimeric Fc domain and wherein the scFv has a first antigen binding specificity; and a light chain (LC) transfected separately so that Fab domains having a second antigen binding specificity are formed with the two VH domains. Sequences for illustrative CEACAM5 x CD28 bsAbs in the 2+1 Fab₂-scFv-Fc format are depicted in FIG. 49.

Although the above provide particularly useful backbones, any number of heterodimerization approaches as is known in the art could find use in the bispecific formats, in combination with any number of approaches for purifying heterodimers from contaminating homodimers, including those depicted in FIGS. 3. Any of the number of linkers as is known in the art may find use in linking the VH and VL domains of the scFv.

Finally, it may be useful to maximize serum half-life of the bsAbs, and any of the number of half-life extending variants as is known in the art may find use in these bsAbs. Regardless of bsAb format, the CD28 bispecific antibodies are monovalent for CD28 and incorporate Fc variants engineered to ablate FeyR binding to avoid potential superagonism. Such Fc variants include those depicted in FIG. 5.

### Example 5: STEAP1 x CD28 bsAbs Combine Productively with CD3 bsAbs

As described in Example 4, the STEAP1 x CD28 bsAbs are contemplated for combination with CD3 bsAbs. Such CD3 bsAbs may utilize the CD3 binding domains as depicted in FIG. 25. As an example, the STEAP1 x CD28 bsAbs may be combined with STEAP1 x CD3 bsAbs (see e.g., U.S. Pat. Nos. 9,982,063, 10,781,258, WO 2021/030586, WO 2021/041300, WO 2021/053587, and WO 2021/098822). Alternatively, the STEAP1 x CD28 bsAbs may be combined with CD3 bsAbs targeting other antigens. 5A: Combination with EpCAM x CD3

To investigate, 10,000 STEAP1+22Rv1 tumor cells (-90K STEAP1 antigens/cell) were seeded per well in a plate. The next day, purified T cells were added at a 1:10 effector to target ratio and incubated with 1 μg/ml illustrative EpCAM x CD3 bsAb and STEAP1 x CD28 bsAb XENP44652 at the concentrations indicated in FIG. 26. After 24 hours at 37° C., cell supematants were assayed by MSD for IL-2 (Meso Scale Discovery, Rockville, Md.). Data as depicted in FIG. 26 illustrates that STEAP1 x CD28 bsAb XENP44652 enhances IL-2 secretion driven by the EpCAM x CD3 bsAb. 5B: Combination with B7H3 x CD3

In an additional experiment, 10,000 STEAP1+22Rv1 tumor cells or wild type 22Rv1 cells were seeded per well in a plate. The following day, T-cells were seeded at an effector to target ratio of 1:1 in the presence of 1 μg/ml illustrative B7H3 x CD3 bsAb and XENP46214 (a STEAP1 x CD28 bsAb in the 1+1 Fab-scFv-Fc format) at the dose range indicated in FIG. 29. IL-2 was assessed after 1 day with MSD (Meso Scale Discovery, Rockville, Md.). As depicted in FIG. 29, when combined with a B7H3 x CD3 engager, XENP46214 is able to conditionally enhance anti-tumor response on target cells with higher levels of STEAP1 expression.

Furthermore, an experiment was conducted in which co-cultures of T-cells and STEAP1+22Rv1 tumor cells (1:1 E:T ratio) were treated with 0.5 pg/mL of an illustrative B7H3 x CD3 bsAb, either alone or in combination with 1 μg/mL of a STEAP1 x CD28 bsAb.

Cell killing was tracked and analyzed over a period of 6 days via Incucyte. As illustrated in FIG. 33, when combined with a B7H3 x CD3 bsAb, STEAP1 x CD28 bsAbs significantly enhanced target cell killing. Additionally, XENP46214, having the STEAP-A L1.56 affinity matured variable light domain, showed improved target cell killing compared to XENP44652, having the parental humanized STEAP-A H1L1 binding domain. 5C: Combination with PSMA x CD3

FIG. 35A illustrates the productive combination of STEAP1 x CD28 bsAbs with an illustrative PSMA x CD3 bsAb. Co-cultures of T cells and 22Rv1 cells (having—12,000 STEAP1 antigens, 120,000 B7H3 antigens, and 30,000 PSMA antigens) were seeded at a 1:1 E:T ratio and were treated with 1 μg/mL of the indicated CD28 bsAb in combination with 10 μg/mL of illustrative PSMA x CD3 bsAb. Cell killing was tracked and analyzed over time via Incucyte. STEAP1 x CD28 bsAbs XENP47235 and XENP47241 induced significantly better tumor cell killing when compared with illustrative B7H3 x CD28 bsAb. 5D: Combination with STEAP1 x CD3

FIG. 35B illustrates the productive combination of STEAP1 x CD28 bsAbs with an illustrative STEAP1 x CD3 bsAb. Co-cultures of T cells and 22Rv1 cells (having—12,000 STEAP1 antigens, 120,000 B7H3 antigens, and 30,000 PSMA antigens) were seeded at a 1:1 E:T ratio and were treated with 1 μg/mL of the indicated CD28 bsAb in combination with 10 μg/mL of illustrative STEAP1 x CD3 bsAb. Cell killing was tracked and analyzed over time via Incucyte. STEAP1 x CD28 bsAbs XENP47235 and XENP47241 induced significantly better tumor cell killing when compared with illustrative B7H3 x CD28 bsAb.

### Example 6: Tuning STEAP1 Binding Valency

STEAP1 x CD28 bsAbs were engineered in 2+1 formats (mAb-scFv or Fab₂-scFv-Fc) to investigate the effects of bivalent binding to STEAP1 antigen compared with monovalent binding of a STEAP1 x CD28 bsAb in the 1+1 Fab-scFv format. LOX-IMVI cells (which express both STEAP1 at—8000 antigens/cell as well as an aCD3-scFv in order to provide Signal 1) were mixed with purified T cells at a 1:1 E:T ratio. As depicted in FIG. 36, XENP47235 in the 1+1 Fab-scFv-Fc format reaches a higher level of binding as measured by the MFI, indicating a higher antigen binding capacity and potentially higher efficacy. However, XENP47241 in the 2+1 mab-scFv displayed avid binding and a more potent induction of IL2. Each format may be advantageous in different contexts.

### Example 7: Further characterization of STEAP1 x CD28 bsAbs

In a further experiment, the induction of IL2 secretion from purified T cells incubated with 22RV1-STEAP1 tumor cells at a 1:1 effector:target ratio and 1 ug/ml of illustrative B7H3 x CD3, PSMAx CD3, or STEAP1 x CD3 bispecific and dose escalation of STEAP1 x CD28 bsAb was measured as depicted in FIG. 37. T cell proliferation was measured as well as indicated by CD4⁺ T cell counts as depicted in FIG. 38. STEAP1 x CD28 bsAbs in the 2+1 mAb-scFv format demonstrate a more potent induction of IL-2 and of CD4⁺ T cell proliferation when compared with the 1+1 Fab-scFv format.

### Example 8: CEACAM5 x CD28 bsAbs Combine Productively with CD3 bsAb

As described in Example 4, the CEACAM5 x CD28 bsAbs are contemplated for combination with CD3 bsAbs. Such CD3 bsAbs may utilize the CD3 binding domains as depicted in FIG. 25. As an example, the CEACAM5 x CD28 bsAbs may be combined with CEACAM5 x CD3 bsAbs (see e.g., U.S. Pat. Nos. 9,982,063, 10,781,258, WO 2021/030586, WO 2021/041300, WO 2021/053587, and WO 2021/098822). Alternatively, the CEACAM5 x CD28 bsAbs may be combined with CD3 bsAbs targeting other antigens.

To investigate, various CEA+B7H3⁺ tumor cells (varied antigen densities) were mixed with purified T cells (various effector:target E:T as indicated) and incubated with dose escalation CEACAM5 x CD28 bsAbs (with various CEACAM5 binding domains) and 1 μg/ml illustrative B7H3 x CD3 bsAb or dose escalation of illustrative B7H3 x CD3 bsAb and 1 μg/ml CEACAM5 x CD28 bsAb for 24 hr at 37° C. Cell supernatant was assayed by MSD for IL-2 (Meso Scale Discovery, Rockville, Md.). Data as depicted in FIGS. 43 and 44 show that each of the CEACAM5 x CD28 bsAbs enhances IL-2 secretion driven by the B7H3 x CD3 bsAb. Notably, CEACAM5 x CD28 bsAb based on the CEA-A was most potent in activity. Further, functional activity is dependent on CEACAM5 density, albeit active over a wide range. Additionally, CEACAM5 x CD28 bsAb are very reduced in efficacy (as indicated by very low IL-2 secretion) on CEACAM51° tumor cell HT29 representing normal tissue suggesting the bsAbs may be well tolerated. In data not shown, it was additionally found that functional activity of CEACAM5 x CD28 is insensitive to the choice of CD3 bispecific.

In another experiment, SNU-C1 tumor cells were mixed with purified T cells (1:1 effector:target ET) and incubated with dose escalation CEACAM5 x CD28 bsAb XENP44338 and 1 μg/ml illustrative B7H3 x CD3 bsAb. T cell counts and CD25 expression were determined via FACS. Data as depicted in FIG. 51 show that CEACAM5 x CD28 bsAb enhance T-cell activation and proliferation.

### Example 9: Tuning CEACAM5 x CD28 bsAbs

9A: Tuning CEACAM5x CD28 bsAbs for activity

### 9A(a): Tuning CD28 Binding Affinity

CEACAM5 x CD28 bsAbs in the 1+1 Fab-scFv-Fc format were engineered with the CEA-A binding domain and a CD28 affinity ladder. HPAF-II (CEA^{high}), MKN45 (CEA^{high}), LS1074, LS174T (CEAmea-iow), or HT-29 (CEAl® ⁰) tumor cells were mixed with purified T cells (1:1 effector:target) and incubated with dose escalation of CEACAM5 x CD28 bsAbs and 1 μg/ml illustrative B7H3 x CD3 bsAb. IL2 secretion was measured after 24 hours, data for which are depicted in FIG. 52. The data show that functional activity is weakly dependent on CD28 affinity. Notably, the tighter CD28 variants (e.g. 37 nM and 96 nM, respectively in XENP44332 and XENP44560) are more active on “normal tissue” proxy (i.e. HT-29).

### 9A(b): Tuning CEACAM5 Binding Valency

CEACAM5 x CD28 bsAbs were engineered in 2+1 formats (mAb-scFv, Fab2-scFv-Fc, or Fab₂-Fc x scFv-Fc) to investigate whether bivalent binding of CEACAM5 may improve activity. In a first set of experiments, HPAF-II (CEA^{high}), SNU-C1 (CEA^{high}), LS1034 (CEA^{med-low}), LS513 (CEA^{med-low}), or LS174T (CEA^{med-low}) tumor cells were mixed with purified T cells (5:1 or 10:1 effector:target) and incubated with dose escalation of CEACAM5 x CD28 bsAbs in the 1+1 Fab-scFv-Fc, 2+1 mAb-scFv, and 2+1 Fab₂-scFv-Fc formats and 1 μg/ml illustrative B7H3 x CD3 bsAb. In another set of experiments, MKN-45, LS174T, Lovo, and HT-29 cells were mixed with purified T cells at 1:1 effector:target ratio and incubated with dose escalation of CEACAM5 x CD28 bsAbs in the 1+1 Fab-scFv-Fc and 2 +1 Fab₂-Fc x scFv-Fc formats and 1 μg/ml illustrative B7H3 x CD3 bsAb. IL2 secretion was measured after 24 hours, data for which are depicted in FIGS. 53 and 71. The data show that the 1+1 format having monovalent CEACAM5 binding induces more IL-2 release compared to the bivalent CEACAM5 binding formats.

9B: Tuning CEACAM5 x CD28 stability

CEACAM5 x CD28 bsAbs in the 1+1 Fab-scFv-Fc format were engineered using disulfide-stabilized CD28 binding domains as described in Example 1A. HPAF-II tumor cells were mixed with purified T cells (1:1 effector:target) and incubated with dose escalation of CEACAM5 x CD28 bsAbs and 1 μg/ml illustrative B7H3 x CD3 bsAb. IL2 secretion was measured after 24 hours. Data as depicted in FIGS. 66 and 70 show that disulfide-stabilizing the higher affinity CD28 binding domains (e.g. 37 nM H1.14_L1.71 and 96 nM H1.1_L1.171) brought their activity in line with or slightly stronger than the lower affinity 180 nM CD28 binding domain (H1_L1.71). As depicted in FIG. 70A-70F, this is irrespective of CEA binding affinity and CEA density on target cell lines.

9C: Tuning CEACAM5 x CD28 bsAbs to mitigate blockade by soluble CEACAM5 (sCEA)
9C(a): Bivalent CEACAM5 binding tolerates sCEACAM5

LS174T tumor cells were mixed with purified T cells (1:1 effector:target) and incubated with dose escalation of CEACAM5 x CD28 bsAbs in the 1+1 Fab-scFv-Fc and 2 +1 Fab₂-Fc x scFv-Fc formats, 1 μg/ml illustrative B7H3 x CD3 bsAb, and increasing concentrations of soluble CEACAM5. IL2 secretion was measured after 24 hours, and EC50 is depicted in FIG. 37. The data show that while the 2+1 format is less efficacious and less potent, it better tolerates soluble CEACAM5 as indicated by higher sCEACAM5 concentrations needed to shift EC50 from baseline. 9C(b): Effect of soluble CEACAM5 is mitigated by reducing CEACAM5 affinity

Based on mechanism-based modeling (showing formation of trimers per T cell i.e. a trimer of bispecific antibody, T cell, and tumor cell), it was predicted that reducing CEACAM5 affinity mitigated effect of soluble CEACAM5 (modeled data depicted in FIG. 54). Notably, 15 nM CEACAM5 binding affinity appeared to be a sweet spot.

In an experiment, HPAF-II tumor cells were incubated with purified T cells and CEACAM5 x CD28 bsAbs having a CEACAM5 affinity ladder, 1 μg/ml B7H3 x CD3, and with or without 1 μg/ml sCEACAM5. IL2 secretion was assessed after 24 hours, data depicted in FIG. 56. Consistent with the modeling, the data show that XENP44338 having the tightest ˜0.7 nM CEACAM5 binding affinity was strongly affected by the sCEACAM5.

XENP44745 having 5 nM CEACAM5 binding affinity was less impacted by sCEACAM5, and XENP44746 having 57 nM CEACAM5 binding affinity was negligibly affected (albeit, much less potent). 9D: Tuning CEACAM5 x CD28 bsAbs to optimize efficacy and sCEACAM5 tolerance

Based on the above findings, it was desired to use the 1+1 format for its efficacy, while tuning the CEACAM5 binding affinity to optimize efficacy and sCEACAM5 tolerance. Accordingly, CEACAM5 x CD28 bsAbs in the 1+1 Fab-scFv-Fc format were engineered with affinity-engineered humanized CEACAM5 binding domains described in Example X. In various experiments, HPAF-II tumor cells were incubated with purified T cells and CEACAM5 x CD28 bsAbs having a CEACAM5 affinity ladder, 1 μg/ml B7H3 x CD3, and with or without 1 μg/ml sCEACAM5. IL2 secretion was assessed after 24 hours, data depicted in FIGS. 57-60. Collectively, the data confirm that increased CEACAM5 affinity correlates with increased potency but also sCEACAM5 dependent IL-2 release blockade. Notably, variants were engineered with −15 nM binding affinity for CEACAM5 which enabled optimized efficacy and sCEACAM5 tolerance.

### 9E: Further Tuning CD28 Binding Affinity

CEACAM5 x CD28 bsAbs having either high affinity 26 nM or low affinity 130 nM CD28 binding domains were incubated with MKN-45 (CEA^{high}) or Lovo (CEACAM51° ^{W}) cells at 1:1 effector:target ratio with 1 μg/ml B7H3 x CD3. IL2 secretion was measured after 6 and 24 hours, data for which are depicted in FIG. 61. XENP45915 having 26 nM CD28 binding affinity was more active at 6 hours and 24 hours in comparison to XENP44915 having 130 nM CD28 binding affinity. However as depicted in FIG. 61B, at 24 hours, the more potent XENP45915 a hooking effect which is especially pronounced on high density tumor cells.

Dose escalation CEA x CD28 bsAb XENP44915 having low affinity 130 nM CD28 binding domain were incubated with MKN45 (CEA^{high}), HPAF-II (CEA^{high}), LS1034 (CEA^{med}), LS174T (CEA^{med}), Lovo (CEA^{med}), and HT29 (CEA° ⁰) cells at 1:1 effector:target ratio and 1 μg/ml illustrative B7H3 x CD3 bsAb IL2 secretion was measured after 24 hours as depicted in FIG. 68 and demonstrate that XENP44915 require CEA expression levels (e.g. med-high) representative of tumors for activity.

Dose escalation CEA x CD28 bsAb XENP44915 having low affinity 130 nM CD28 binding domain were incubated with HPAF-II cells and 0.1 or 1 μg/ml illustrative B7H3 x CD3 bsAb and dose escalation of CEA x CD28 XENP44915. The data in FIG. 69A-69B showing T cell activation and tumor cell killing demonstrate that CEA x CD28 synergizes with high and low concentrations of T-cell engager.

### Example 10: CEACAM5 x CD28 bsAb Combine Productively with PD1 Blockade

Checkpoint blockade (e.g. PD-1 blockade) may be a useful therapeutic modality to stack with engagement of T cell costimulatory receptors on TILs with agonistic antibodies as it would provide broad utility in solid tumors and circumvent CTLA4 inhibition of CD28 pathway. Accordingly, the combination of CEACAM5 x CD28 bispecific antibodies XENP44915 and XENP45560 with XENP16432 (a bivalent anti-PD-1 mAb based on the variable regions of nivolumab) was investigated. Dose titration of CEACAM5 x CD28 bsAb were incubated with illustrative PD-1 mAb and LS1304 cancer cells at 10:1 effector:target ratio. IL2 secretion after 24 hours are depicted in FIG. 62, and show that combining CEACAM5 x CD28 bsAb combines productively with PD-1 blockade.

### Example 11: CEA x CD28 Promotes T-Cell Activation by TCR-pMHC Recognition

A431-02M-null cells stably expressing CEACAM5 were engineered to express a CMV-derived (HLA-A2/NLVa) or a melanoma-derived (HLA-A2/G209; negative control) pMHC antigenic complex. Cells were cocultured with PBMCs from an HLA-A2⁺, CMV-seropositive donor, and treated with a dose titration of CEACAM5 x CD28 mAb. Data showing IL2 secretion is depicted in FIG. 67.

### Example 12: Attenuating CEACAM8 Cross-Reactivity

The CEA-A binding domain exhibits CEACAM8 cross-reactivity. Notably as depicted in Figure X, while humanizing the CEA-A binding domain reduces CEACAM5 binding affinity, CEACAM8 binding affinity is also reduced (see Octet sensorgram as depicted in FIG. 79). However, in restoring CEACAM5 binding affinity as in H1.7_L2.15, some CEACAM8 affinity is also restored. Accordingly, H1.7_L2.15 were further engineered with substitutions in the variable heavy and/or variable light domain. FIG. 80 depicts binding affinity of illustrative such further variants and show that CEA-A_H1.13_L2.15 maintained similar CEACAM5 affinity as H1.7_L2.15 along with drastically reduced CEACAM8 affinity.

To confirm that the new CEACAM8 affinity reduced variants retained CEACAM5 dependent functional activity and tolerance to shed CEA, 8K HPAF-II or LS174T tumor cells were seeded, and after 24 hours, mixed with purified T cells at 1:1 effector:target ratio and incubated with dose escalation of CEA x CD28 and 1 μg/mL illustrative B7H3 x CD3 bsAb with or without 1 μg/mL sCEA for 24 hour at 37° C. Cell supernatants were assayed for IL2 release, data for which are depicted in Figure X, confirming that the new CEACAM8 affinity-reduced variants retained CEACAM5 dependent functional activity and tolerance to shed CEA.

### Example 13: Further Characterizing CEACAM5 x CD28 bsAbs

As shown in the foregoing examples, the CEA-A binding domain is more active than other CEA binding domain investigated, and the H1.13_L2.15 variant has an affinity that balances potency and sCEA tolerance with attenuated CEACAM8 cross-reactivity. As noted in Example 9E, CEACAM5 x CD28 bsAbs having different CD28 affinities were both active in vivo, albeit XENP45915 having 26 nM CD28 binding affinity was more active in comparison to XENP44915 having 130 nM CD28 binding affinity. Thus on the one hand, 26 nM CD28 may potentially be more active with TCR-pMHC Signal 1, more active with reduced CD28 expression (e.g. tumor infiltrating lymphocytes), and more active on CEA-low tumors; on the other hand, 130 nM CD28 may potentially be less active on CEA+ normal tissues and this lower potency may allow higher dose levels to be reached, overcoming any TMDD. Accordingly, this section further characterize CEACAM5 x CD28 bsAbs having the CEA-A H1.13_L2.15 binding domain in combination with the 26 nM or 130 nM CD28 binding domains (respectively XENP46322 and XENP45906).

13A: Both XENP46322 and XENP45906 Require Similar Threshold CEACAM5 densities for activity corresponding to CEA densities found in CRC patients

8K tumor cells having a range of CEACAM5 densities were seeded. After 24 hours, the tumor cells were mixed with purified T cells at a 1:1 effector:target ratio and incubated with varying concentration of XENP46322 or XENP45906 and 1 μg/ml illustrative EpCAM x CD3 bsAb for 24 hour at 37° C. Cell supernatant was assayed for IL2 release, data for which are depicted in FIG. 72.

### 13B: Both XENP46322 and XENP45906 Potentiate CD3 T Cell Engager Induced Cytotoxicity

In a first experiment, 20K tumor cells (LS-174T or Lovo) were mixed with purified T cells at a 1:1 effector:target ratio and incubated with dose titration of illustrative B7H3 x CD3 bsAb and fixed 1 μg/mL concentration of CEA x CD28 for 48 hours at 37° C. Cells were stained with Zombie Aqua flow Ab for 45 minutes at 4° C. followed by fixation and analysis by FACS, data for which are depicted in FIG. 73.

In another experiment, HPAF-II were co-cultured with purified T cells and either EpCAM x CD3 only or in combination with CEACAM5 x CD28 bsAbs. Cell growth was assessed using xCELLigence, data for which are depicted in FIG. 74.

Collectively, the data show that both XENP46322 and XENP45906 potentiate activity of various CD3 T cell engager and induce cytotoxicity.

13C: Both XENP46322 and XENP45906 Function in Synergy with TCR-pMHC Signal 1 and Combine with PD1 Blockade

20K A431 B2M KO PGK-NLV CEA knock-in cells were seeded. After 24 hours, the cells were mixed with purified CMV reactive T cells at 10:1 effector:target ratio and incubated with varying concentrations of CEACAM5 x CD28 bsAbs alone or in combination with anti-PD1. Cell supernatant ere assayed for IL2 release, data for which are depicted in FIG. 75.

13D: Both XENP46322 and XENP45906 have Similar Tolerance to Soluble CEA

8K HPAF-II cells were seeded, and after 24 hours, mixed with purified T cells at 1:1 effector:target ratio and incubated with dose titration of CEACAM5 x CD28 bsAb and 1 μg/ml illustrative B7H3 x CD3, with and without, soluble human CEA for 24 hour at 37° C. Cell supernatants were assayed for IL2, data for which are depicted in FIG. 76.

### 13E: Both XENP46322 and XENP45906 Mediate Anti-Tumor Response and T Cell Expansion In Vivo

NSG mice that were MHC I/II-DKO (NSG-DKO) and thus resistant to GVHD were used. NSG-DKO mice (8 or 10 per group) were intradermally inoculated with 1×10⁶ HPAFII cells on Day −14. Mice were then intraperitoneally injected with 5×10⁶ human PBMCs and treated with the indicated test articles/test article combinations on Day 0, and further treated with the indicated test articles on Days 7, 14, 21, and 28. Tumor volumes were monitored by caliper measurements once to three times per week, data for which are shown (days post 1^{st} dose) in Figures X-Y Blood was drawn weekly and analyzed by flow cytometry to count expansion of human lymphocytes, data for which are shown in Figure X.

The data show that each of the CEACAM5 x CD28 bsAbs demonstrated anti-tumor activity in combination with EpCAM x CD3 bsAb. Notably, higher dose XENP46322 saw an inverse dose response suggesting overactivation of T cells by the higher affinity CD28 binding domain.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

1.-185. (canceled)

186. An anti-STEAP1 x anti-CD28 bispecific antibody comprising:
a) a means for binding STEAP1 ; and
b) a means for binding CD28.

187. A heterodimeric antibody comprising:
a) a first monomer comprising:
i) a single chain variable fragment (scFv); and
ii) a first Fc domain, wherein the scFv is covalently attached to the N-terminus of the first Fc domain using a domain linker;
b) a second monomer comprising, from N-terminal to C-terminal, a VH1-CH1-hinge-CH2-CH3, wherein VH1 is a first variable heavy domain and CH2-CH3 is a second Fc domain; and
c) a light chain comprising, from N-terminal to C- terminal, VL1-CL, wherein VL1 is a first variable light domain and CL is a constant light domain,
wherein the scFv comprises a second VH domain (VH2), a scFv linker, and a second variable light domain (VL2),
wherein the VH1 and the VL1 together form a first antigen binding domain (ABD) and the VH2 and the VL2 together form a second ABD, and
wherein one of the first ABD and the second ABD is a CD28 binding domain and the other of the first ABD and second ABD is a Six Transmembrane Epithelial Antigen of Prostate 1 (STEAP1 ) binding domain.

188. The heterodimeric antibody according to claim 187, wherein the first ABD is the STEAP1 binding domain and the second ABD is the CD28 binding domain.

189. The heterodimeric antibody according to claim 188, wherein VH1 has an amino acid sequence as set forth in any one of SEQ ID NOs: 463, 471, and 1466-1696, and VL1 has an amino acid sequence as set forth in any one of SEQ ID Nos: 467, 475, and 1697-1815.

190. The heterodimeric antibody according to claim 188, wherein VH2 has an amino acid sequence as set forth in any one of SEQ ID NOs: 106, and 116-184-and VL2 has an amino acid sequence as set forth in any one of SEQ ID Nos: 110, 185-290, and 2679.

191. A nucleic acid composition comprising:
a) a first nucleic acid encoding the first monomer of claim 187;
b) a second nucleic acid encoding the second monomer of claim 187; and
c) a third nucleic acid encoding the light chain of claim 187.

192. An expression vector composition comprising:
a) a first expression vector comprising a first nucleic acid encoding the first monomer of claim 187;
b) a second expression vector comprising a second nucleic acid encoding the second monomer of claim 187; and
c) a third expression vector comprising a third nucleic acid encoding the light chain of claim 187.

193. A host cell comprising the expression vector composition of claim 192.

194. A method of making a heterodimeric antibody comprising culturing the host cell of claim 193 under conditions wherein the heterodimeric antibody is expressed and recovering the heterodimeric antibody.

195. A heterodimeric antibody comprising:
a) a first monomer comprising from N-terminal to C-terminal, VH1-CH1-first domain linker-scFv-second domain linker-CH2-CH3,
wherein VH1 is a first variable heavy domain, and CH2-CH3 is a first Fc domain;
b) a second monomer, comprising from N-terminal to C-terminal, a VH1-CH1-hinge-CH2-CH3, wherein CH2-CH3 is a second Fc domain;
c) a first light chain comprising, from N-terminal to C-terminal, VL1-CL, wherein VL1 is a first variable light domain and CL is a constant light domain; and
d) a second light chain comprising, from N-terminal to C-terminal, VL1-CL, wherein VL1 is a first variable light domain and CL is a constant light domain,
wherein the scFv comprises a second VH domain (VH2), a scFv linker, and a second variable light domain (VL2),
wherein the VH1 of the first monomer and the VL1 of the first light chain and the VH1 of the second monomer and the VL1 of the second light chain each form a first antigen binding domain (ABD), and the VH2 and the VL2 form a second ABD, and
wherein the first ABDs are Six Transmembrane Epithelial Antigen of Prostate 1 (STEAP1 ) binding domains and the second ABD is a CD28 binding domain, or the first ABDs are CD28 binding domains and the second ABD is a STEAP1 binding domain.

196. The heterodimeric antibody according to claim 195, wherein the first ABDs are the STEAP1 binding domains and the second ABD is the CD28 binding domain.

197. The heterodimeric antibody according to claim 196, wherein the VH1 of the first monomer and the VH1 of second monomer each have an amino acid sequence as set forth in any one of SEQ ID NOs: 463, 471, and 1466-1696, and the VL1 of the first light chain and the VL1 of the second light chain each have an amino acid sequence as set forth in any one of SEQ ID Nos: 467, 475, and 1697-1815.

198. The heterodimeric antibody according to claim 196, wherein VH2 has an amino acid sequence as set forth in any one of SEQ ID NOs: 106, and 116-184-and VL2 has an amino acid sequence as set forth in any one of SEQ ID Nos: 110, 185-290, and 2679.

199. A nucleic acid composition comprising:
a) a first nucleic acid encoding the first monomer of claim 195;
b) a second nucleic acid encoding the second monomer of claim 195; and
c) a third nucleic acid encoding the first light chain and second light chain of claim 195

200. An expression vector composition comprising:
a) a first expression vector comprising a first nucleic acid encoding the first monomer of claim 195;
b) a second expression vector comprising a second nucleic acid encoding the second monomer of claim 195; and
c) a third expression vector comprising a third nucleic acid encoding the first light chain and second light chain of claim 195.

201. A host cell comprising the expression vector composition of claim 200.

202. A method of making a heterodimeric antibody comprising culturing the host cell of claim 201 under conditions wherein the heterodimeric antibody is expressed and recovering the heterodimeric antibody.

203. A STEAP1 binding domain, wherein the STEAP1 binding domain comprises:
a) a variable heavy domain (VH) having an amino acid sequence as set forth in any one of SEQ ID NOs: 463, 471, and 1466-1696; and
b) a variable light domain (VL) having an amino acid sequence as set forth in any one of SEQ ID Nos: 467, 475, and 1697-1815.

204. A nucleic acid composition comprising a first nucleic acid encoding the VH of claim 203; and a second nucleic acid encoding the VL of claim 203.

205. An expression vector composition comprising a first expression vector comprising a first nucleic acid encoding the VH of claim 203; and a second expression vector comprising a second nucleic acid encoding the VL of claim 203.

206. A host cell comprising the expression vector composition of claim 205.

207. A method of making a STEAP1 binding domain comprising culturing the host cell of claim 206 under conditions wherein the heterodimeric antibody is expressed and recovering the STEAP1 binding domain.
